(19) United States
(12) Patent Application Publication
(10) Pub. No.: US 2025/0059198 A1
(43) Pub. Date: Feb. 20, 2025

(54) BRM DEGRADING COMPOUNDS AND ASSOCIATED METHODS OF USE

(71) Applicants: Arvinas Operations, Inc., New Haven, CT (US); Genentech, Inc., South San Francisco, CA (US)

(72) Inventors: Michael Berlin, Flemington, NJ (US); Fabio Broccatelli, South San Francisco, CA (US); Huifen Chen, South San Francisco, CA (US); Peter Scott Dragovich, South San Francisco, CA (US); Jing Wang, Milford, CT (US)

(21) Appl. No.: 18/710,130

(22) PCT Filed: Nov. 23, 2022
(86) PCT No.: PCT/US2022/050943

§ 371 Date: May 14, 2024

Related U.S. Application Data
(60) Provisional application No. 63/282,897, filed on Nov. 24, 2021.

(51) Int. Cl. C07D 487/08; A61K 31/501; A61K 31/5377; A61K 31/5386; A61K 31/551; C07D 413/14; C07D 417/14; C07D 498/10

(52) U.S. Cl. CPC C07D 487/08; A61K 31/501; A61K 31/5377; A61K 31/5386; A61K 31/551; C07D 413/14; C07D 417/14; C07D 498/10

(57) ABSTRACT

The present disclosure relates to bifunctional compounds, which find utility as modulators of SMARCA2 or BRM (target protein). In particular, the present disclosure is directed to bifunctional compounds, which contain on one end a ligand that binds to the Von Hippel-Lindau E3 ubiquitin ligase, and on the other end a moiety which binds the target protein, such that the target protein is placed in proximity to the ubiquitin ligase to effect degradation (and inhibition) of target protein. The present disclosure exhibits a broad range of pharmacological activities associated with degradation/inhibition of target protein. Diseases or disorders that result from aggregation or accumulation of the target protein are treated or prevented with compounds and compositions of the present disclosure.

### RELATED APPLICATIONS

This application claims priority to U.S. Provisional Application No. 63/282,897, filed on Nov. 24, 2021. The entire contents of the foregoing application are expressly incorporated herein by reference.

### FIELD OF THE INVENTION

The description provides bifunctional compounds comprising a target protein binding moiety and a E3 ubiquitin ligase binding moiety, and associated methods of use. The bifunctional compounds are useful as modulators of targeted ubiquitination, especially with respect to Switch/Sucrose Non Fermentable (SWI/SNF)-Related, Matrix-Associated, Actin-Dependent Regulator of Chromatin, Subfamily A, Member 2 (SMARCA2) (i.e., BRAHMA or BRM), which are degraded and/or otherwise inhibited by bifunctional compounds according to the present disclosure.

### BACKGROUND

Most small molecule drugs bind enzymes or receptors in tight and well-defined pockets. On the other hand, protein-protein interactions are notoriously difficult to target using small molecules due to their large contact surfaces and the shallow grooves or flat interfaces involved. E3 ubiquitin ligases (of which hundreds are known in humans) confer substrate specificity for ubiquitination, and therefore, are more attractive therapeutic targets than general proteasome inhibitors due to their specificity for certain protein substrates. The development of ligands of E3 ligases has proven challenging, in part due to the fact that they must disrupt protein-protein interactions. However, recent developments have provided specific ligands which bind to these ligases. For example, since the discovery of nutlins, the first small molecule E3 ligase inhibitors, additional compounds have been reported that target E3 ligases but the field remains underdeveloped. For example, since the discovery of Nutlins, the first small molecule E3 ligase mouse double minute 2 homolog (MDM2) inhibitors, additional compounds have been reported that target MDM2 (i.e., human double minute 2 or HDM2) E3 ligases (J. Di, et al. Current Cancer Drug Targets (2011), 11(8), 987-994).

One E3 ligase with exciting therapeutic potential is the von Hippel-Lindau (VHL) tumor suppressor, the substrate recognition subunit of the E3 ligase complex VCB, which also consists of elongins B and C, Cul2 and Rbx1. The primary substrate of VHL is Hypoxia Inducible Factor 1α (HIF-1α), a transcription factor that upregulates genes such as the pro-angiogenic growth factor VEGF and the red blood cell inducing cytokine erythropoietin in response to low oxygen levels. The first small molecule ligands of Von Hippel Lindau (VHL) to the substrate recognition subunit of the E3 ligase were generated, and crystal structures were obtained confirming that the compound mimics the binding mode of the transcription factor HIF-1α, the major substrate of VHL.

Bifunctional compounds such as those that are described in U.S. Patent Application Publications 2015-0291562 and 2014-0356322 (incorporated herein by reference), function to recruit endogenous proteins to an E3 ubiquiuin ligase for degradation. In particular, the publications describe bifunctional or proteolysis targeting chimeric (PROTAC) compounds, which find utility as modulators of targeted ubiquitination of a variety of polypeptides and other proteins, which are then degraded and/or otherwise inhibited by the bifunctional compounds.

The Switch/Sucrose Non Fermentable (SWI/SNF) is a multi-subunit complex that modulates chromatic structure through the activity of two mutually exclusive helicase/ATPase catalytic subunits SWI/SNF-Related, Matrix-Associated, Actin-Dependent Regulator of Chromatin, Subfamily A, Member 2 (SMARCA2, BRAHMA or BRM) and SWI/SNF-Related, Matrix-Associated, Actin-Dependent Regulator of Chromatin, Subfamily A, Member 4 (SMARCA4 or BRG1). The core and the regulatory subunits couple ATP hydrolysis to the perturbation of histone-DNA contacts, thereby providing access points to transcription factors and cognate DNA elements that facilitate gene activation and repression.

Mutations in the genes encoding the twenty canonical SWI/SNF subunits are observed in nearly 20% of all cancers with the highest frequency of mutations observed in rhabdoid tumors, female cancers (including ovarian, uterine, cervical and endometrial), lung adenocarcinoma, gastric adenocarcinoma, melanoma, esophageal, and renal clear cell carcinoma. Despite having a high degree of homology, and their presumed overlapping functions, SMARCA2 and SMARCA4 have been reported as having different roles in cancer. For example, SMARCA4 is frequently mutated in primary tumors, while SMARCA2 inactivation is infrequent in tumor development. In fact, numerous types of cancer have been shown to be SMARCA4-related (e.g., cancers having a SMARCA4-mutation or a SMARCA4-deficiency, such as lack of expression), including, e.g., lung cancer (such as non-small cell lung cancer).

SMARCA2 has been demonstrated as one of the top essential genes in SMARCA4-related or-mutant cancer cell lines because SMARCA4-deficient patient populations or cells depend exclusively on SMARCA2 activity—i.e., there is a greater incorporation of SMARCA2 into the complex to compensate for the SMARCA4 deficiency. Thus, SMARCA2 may be targeted in SMARCA4-related/deficient cancers. The co-occurrence of the deficiency of the expression of two (or more) genes that leads to cell death is known as, synthetic lethality. Accordingly, synthetic lethality can be leveraged in the treatment of certain SMARCA2/SMARCA4-related cancers.

There is an ongoing need for effective treatment for diseases that are treatable by inhibiting or degrading SMARCA2 (i.e., BRAHMA or BRM). However, non-specific effects and the inability to target and modulate SMARCA2 remain obstacles to developing effective treatments. As such, small-molecule therapeutic agents that target SMARCA2 and that leverage or potentiate VHL's substrate specificity would be very useful.

### SUMMARY

The present disclosure describes. bifunctional compounds which function to recruit endogenous proteins to an E3 ubiquitin ligase for degradation, and methods of using the same. In particular, the present disclosure provides bifunctional or proteolysis targeting chimeric (PROTAC) compounds, which find utility as modulators of targeted ubiquitination of a variety of polypeptides and other proteins, which are then degraded and/or otherwise inhibited by the bifunctional compounds as described herein. An advantage of the compounds provided herein is that a broad range of pharmacological activities is possible, consistent with the degradation/inhibition of targeted polypeptides from virtually any protein class or family. In addition, the description provides methods of using an effective amount of the compounds as described herein for the treatment or amelioration of a disease condition, such as cancer, e.g., SMARCA4-related/deficient cancer, such as lung cancer or non-small cell lung cancer.

As such, in one aspect the disclosure provides bifunctional or PROTAC compounds, which comprise an E3 ubiquitin ligase binding moiety (i.e., a ligand for an E3 ubquitin ligase or “ULM” group), and a moiety that binds a target protein (i.e., a protein/polypeptide targeting ligand or “PTM” group) such that the target protein/polypeptide is placed in proximity to the ubiquitin ligase to effect degradation (and inhibition) of that protein. In a preferred embodiment, the ULM (ubiquitination ligase modulator) can be Von Hippel-Lindau E3 ubiquitin ligase (VHL) binding moiety (VLM). For example, the structure of the bifunctional compound can be depicted as:

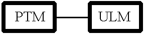

The respective positions of the PTM and ULM moieties as well as their number as illustrated herein is provided by way of example only and is not intended to limit the compounds in any way. As would be understood by the skilled artisan, the bifunctional compounds as described herein can be synthesized such that the number and position of the respective functional moieties can be varied as desired.

In certain embodiments, the bifunctional compound further comprises a chemical linker (“L”). In this example, the structure of the bifunctional compound can be depicted as:

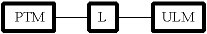

where PTM is a protein/polypeptide targeting moiety, L is a linker, e.g., a bond or a chemical group coupling PTM to ULM, and ULM is a Von Hippel-Lindau E3 ubiquitin ligase (VHL) binding moiety (VLM).

For example, the structure of the bifunctional compound can be depicted as:

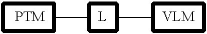

wherein: PTM is a protein/polypeptide targeting moiety; “L” is a linker (e.g. a bond or a chemical linker group) coupling the PTM and a VLM, wherein VLM is Von Hippel-Lindau E3 ubiquitin ligase binding moiety that binds to VHL E3 ligase.

In certain embodiments, the compounds as described herein comprise multiple independently selected ULMs, multiple PTMs, multiple chemical linkers or a combination thereof.

In additional embodiments, VLM can be hydroxyproline or a derivative thereof. Furthermore, other contemplated VLMs are included in U.S. Patent Application Publication No. 2014/03022523, which as discussed above, is incorporated herein in its entirety.

In certain embodiments, “L” is a bond. In additional embodiments, the linker “L” is a connector with a linear non-hydrogen atom number in the range of 1 to 20. The connector “L” can contain, but not limited to the functional groups such as ether, amide, alkane, alkene, alkyne, ketone, hydroxyl, carboxylic acid, thioether, sulfoxide, and sulfone. The linker can contain aromatic, heteroaromatic, cyclic, bicyclic and tricyclic moieties. Substitution with halogen, such as Cl, F, Br and I can be included in the linker. In the case of fluorine substitution, single or multiple fluorines can be included.

In certain embodiments, VLM is a derivative of trans-3-hydroxyproline, where both nitrogen and carboxylic acid in trans-3-hydroxyproline are functionalized as amides.

In an additional aspect, the description provides therapeutic compositions comprising an effective amount of a compound as described herein or salt form thereof, and a pharmaceutically acceptable carrier. The therapeutic compositions modulate protein degradation and/or inhibition in a patient or subject, for example, an animal such as a human, and can be used for treating or ameliorating disease states or conditions which are modulated through the degraded/inhibited protein. In certain embodiments, the therapeutic compositions as described herein may be used to effectuate the degradation of proteins of interest for the treatment or amelioration of a disease, e.g., cancer (including at least one of SWI/SNF associated cancer, a cancer with a SMARCA4 mutation, a cancer with a SMARCA4-deficiency, or a combination thereof), such as lung cancer (e.g., non-small cell lung cancer). In yet another aspect, the present disclosure provides a method of ubiquitinating/degrading a target protein in a cell. In certain embodiments, the method comprises administering a bifunctional compound as described herein comprising a VLM, preferably linked through a linker moiety, as otherwise described herein, wherein the VLM is coupled to the PTM through a linker to target a protein for degradation. Degradation of the target protein will occur when the target protein is placed in proximity to the E3 ubiquitin ligase, thus resulting in degradation/inhibition of the effects of the target protein and the control of protein levels. The control of protein levels afforded by the present disclosure provides treatment of a disease state or condition, which is modulated through the target protein by lowering the level of that protein in the cells of a patient.

In still another aspect, the description provides methods for treating or ameliorating a disease, disorder or symptom thereof in a subject or a patient, e.g., an animal such as a human, comprising administering to a subject in need thereof a composition comprising an effective amount, e.g., a therapeutically effective amount, of a compound as described herein or salt form thereof, and a pharmaceutically acceptable carrier, wherein the composition is effective for treating or ameliorating the disease or disorder or symptom thereof in the subject.

In another aspect, the description provides methods for identifying the effects of the degradation of proteins of interest in a biological system using compounds according to the present disclosure.

The preceding general areas of utility are given by way of example only and are not intended to be limiting on the scope of the present disclosure and appended claims. Additional objects and advantages associated with the compositions, methods, and processes of the present disclosure will be appreciated by one of ordinary skill in the art in light of the instant claims, description, and examples. For example, the various aspects and embodiments of the disclosure may be utilized in numerous combinations, all of which are expressly contemplated by the present description. These additional aspects and embodiments are expressly included within the scope of the present disclosure. The publications and other materials used herein to illuminate the background of the disclosure, and in particular cases, to provide additional details respecting the practice, are incorporated by reference.

### BRIEF DESCRIPTION OF THE DRAWINGS

The accompanying drawings, which are incorporated into and form a part of the specification, illustrate several embodiments of the present disclosure and, together with the description, serve to explain the principles of the disclosure. The drawings are only for the purpose of illustrating an embodiment of the disclosure and are not to be construed as limiting the disclosure. Further objects, features and advantages of the disclosure will become apparent from the following detailed description taken in conjunction with the accompanying figures showing illustrative embodiments of the disclosure, in which:

FIGS. 1A and 1B. Illustration of general principle for PROTAC function. (A) Exemplary PROTACs comprise a protein targeting moiety (PTM; darkly shaded rectangle), a ubiquitin ligase binding moiety (ULM; lightly shaded triangle), and optionally a linker moiety (L; black line) coupling or tethering the PTM to the ULM. (B) Illustrates the functional use of the PROTACs as described herein. Briefly, the ULM recognizes and binds to a specific E3 ubiquitin ligase, and the PTM binds and recruits a target protein bringing it into close proximity to the E3 ubiquitin ligase. Typically, the E3 ubiquitin ligase is complexed with an E2 ubiquitin-conjugating protein, and either alone or via the E2 protein catalyzes attachment of ubiquitin (dark circles) to a lysine on the target protein via an isopeptide bond. The poly-ubiquitinated protein (far right) is then targeted for degradation by the proteosomal machinery of the cell.

### DETAILED DESCRIPTION

The following is a detailed description provided to aid those skilled in the art in practicing the present disclosure. Those of ordinary skill in the art may make modifications and variations in the embodiments described herein without departing from the spirit or scope of the present disclosure. All publications, patent applications, patents, figures and other references mentioned herein are expressly incorporated by reference in their entirety.

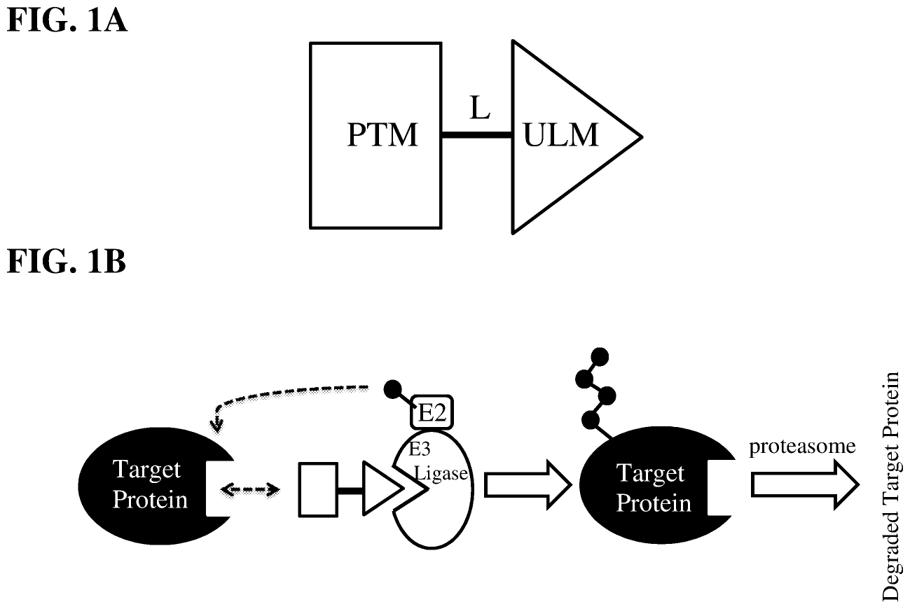

Presently described are compositions and methods that relate to the surprising and unexpected discovery that an E3 ubiquitin ligase protein (e.g., Von Hippel-Lindau E3 ubiquitin ligase (VHL)) ubiquitinates a target protein once it and the target protein are placed in proximity by a bifunctional or chimeric construct that binds the E3 ubiquitin ligase protein and the target protein. Accordingly, the present disclosure provides such compounds and compositions comprising an E3 ubiquintin ligase binding moiety (“ULM”) coupled to a protein target binding moiety (“PTM”), which result in the ubiquitination of a chosen target protein and leads to degradation of the target protein by the proteasome (see FIG. 1). The present disclosure also provides a library of compositions and uses thereof.

In certain aspects, the present disclosure provides compounds that comprise a ligand, e.g., a small molecule ligand (i.e., having a molecular weight of below 2,000 Daltons, 1,000 Daltons, 500 Daltons, or 200 Daltons) that is capable of binding to a ubiquitin ligase, such as VHL. The compounds also comprise a moiety that is capable of binding to target protein in such a way that the target protein is placed in proximity to the ubiquitin ligase to effect degradation (and/or inhibition) of that protein. As disclosed herein, the term “small molecule” can mean, in addition to the above, thea molecule is non-peptidyl (i.e., a molecule, that is, it is not generally considered a peptide, e.g., comprises fewer than 4 amino acids, 3 amino acids, or 2 amino acids). In accordance with the present disclosure, the PTM, ULM, or bifunctional compounds disclosed herein can be a small molecule.

### Definitions

Unless otherwise defined, all technical and scientific terms used herein have the same meaning as commonly understood by one of ordinary skill in the art to which this disclosure belongs. The terminology used in the description is for describing particular embodiments only and is not intended to be limiting of the disclosure.

Where a range of values is provided, it is understood that each intervening value, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either both of those included limits are also included in the disclosure.

The following terms are used to describe the present disclosure. In instances where a term is not specifically defined herein, that term is given an art-recognized meaning by those of ordinary skill applying that term in context to its use in describing the present disclosure.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, in certain methods described herein that include more than one step or act, the order of the steps or acts of the method is not necessarily limited to the order in which the steps or acts of the method are recited unless the context indicates otherwise.

The terms “co-administration,” “co-administering” or “combination therapy” refer to both concurrent administration (administration of two or more therapeutic agents at the same time) and time varied administration (administration of one or more therapeutic agents at a time different from that of the administration of an additional therapeutic agent or agents), as long as the therapeutic agents are present in the patient to some extent, preferably at effective amounts, at the same time. In certain preferred aspects, one or more of the present compounds described herein, are coadministered in combination with at least one additional bioactive agent, especially including an anticancer agent. In particularly preferred aspects, the co-administration of compounds results in synergistic activity and/or therapy, including anticancer activity.

The term “compound”, as used herein, unless otherwise indicated, refers to any specific chemical compound disclosed herein and includes tautomers, regioisomers, geometric isomers, and where applicable, stereoisomers, including optical isomers (enantiomers) and other stereoisomers (diastereomers) thereof, as well as pharmaceutically acceptable salts and derivatives, including prodrug and/or deuterated forms thereof where applicable, in context. Deuterated small molecules contemplated are those in which one or more of the hydrogen atoms contained in the drug molecule have been replaced by deuterium. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds. The term also refers, to prodrug forms of compounds which have been modified to facilitate the administration and delivery of compounds to a site of activity. It is noted that in describing the present compounds, numerous substituents and variables associated with same, among others, are described. It is understood by those of ordinary skill that molecules that are described herein are stable compounds. When the bond is shown, both a double bond and single bond are represented or understood within the context of the compound shown and well-known rules for valence interactions.

The term “ubiquitin ligase” refers to a family of proteins that facilitate the transfer of ubiquitin to a specific substrate protein, targeting the substrate protein for degradation. For example, an E3 ubiquitin ligase protein that alone or in combination with an E2 ubiquitin-conjugating enzyme causes the attachment of ubiquitin to a lysine on a target protein, and subsequently targets the specific protein substrates for degradation by the proteasome. Thus, E3 ubiquitin ligase alone or in complex with an E2 ubiquitin conjugating enzyme is responsible for the transfer of ubiquitin to targeted proteins. In general, the ubiquitin ligase is involved in polyubiquitination such that a second ubiquitin is attached to the first; a third is attached to the second, and so forth. Polyubiquitination marks proteins for degradation by the proteasome. However, there are some ubiquitination events that are limited to mono-ubiquitination, in which only a single ubiquitin is added by the ubiquitin ligase to a substrate molecule. Mono-ubiquitinated proteins are not targeted to the proteasome for degradation, but may instead be altered in their cellular location or function, for example, via binding other proteins that have domains capable of binding ubiquitin. Further complicating matters, different lysines on ubiquitin can be targeted by an E3 to make chains. The most common lysine is Lys48 on the ubiquitin chain. This is the lysine used to make polyubiquitin, which is recognized by the proteasome. As used herein, the term “alkyl,” by itself or as part of another substituent, means, unless otherwise stated, a straight or branched chain hydrocarbon radical, having the number of carbon atoms designated (i.e., C₁₋₈ means one to eight carbons). Absent a specific number of carbon atoms, an alkyl group provided herein is assumed to have one to twelve carbons, one to eight carbons, one to six carbons, or one to four carbons. Examples of alkyl groups include methyl, ethyl, n-propyl, iso-propyl, n-butyl, t-butyl, iso-butyl, sec-butyl, n-pentyl, n-hexyl, n-heptyl, n-octyl, and the like. Alkyl groups may be optionally substituted as provided herein. In some embodiments, the alkyl group is C₁₋₆ alkyl; in some embodiments, the alkyl group is C₁₋₄ alkyl.

The term “optionally substituted,” as used in combination with a substituent defined herein, means that the substituent may, but is not required to be, substituted with one or more suitable functional groups or other substituents as provided herein. For example, a substituent may be optionally substituted with one or more of: halo, cyano, C₁₋₆ alkyl, C₃₋₆ cycloalkyl, C₂₋₆ alkenyl, C₂₋₆ alkynyl, halo(C₁₋₆)alkyl, C₁₋₆ alkoxy, halo(C₁₋₆alkoxy), C₁₋₆ alkylthio, C₁₋₆ alkylamino, NH₂, NH(C₁₋₆ alkyl), N(C₁₋₆ alkyl)₂, NH(C₁₋₆ alkoxy), N(C₁₋₆ alkoxy)₂, —C(O)NHC₁₋₆ alkyl, —C(O)N(C₁₋₆ alkyl)₂, —C(O)NH₂, —C(O)C₁₋₆ alkyl, —C(O)₂C₁₋₆ alkyl, —NHCO(C₁₋₆ alkyl), —N(C₁₋₆ alkyl)CO(C₁₋₆ alkyl), —S(O)C₁₋₆ alkyl, —S(O)₂C₁₋₆ alkyl, oxo, phenyl, benzyl, pyridinyl, pyrrazolyl, thiazolyl, isothiazolyl, or other 5 to 6 membered heteroaryl groups. In some embodiments, each of the above optional substituents are themselves optionally substituted by one or two groups.

The term “cycloalkyl,” as used herein, refers to a C₃₋₁₂ cyclic alkyl group, and includes bridged and spirocycles (e.g., adamantine). Examples of cycloalkyl groups include cyclopropyl, cyclobutyl, cyclopentyl, cycloheptyl, cyclohexyl, cycloheptyl, cyclooctyl, bicyclo[2.2.1]heptanyl, bicyclo[3.1.1]heptanyl, bicyclo[4.1.0]heptanyl, spiro[3.3]heptanyl, and spiro[3.4]octanyl. In some embodiments, the cycloalkyl group is a C₃₋₆ cycloalkyl.

The term “akenyl,” as used herein, refers to C₂₋₁₂ alkyl group, wherein at least two of the carbon atoms are sp² hybridized and form a carbon-carbon double bond between them. An alkenyl group provided herein may contain more than one carbon-carbon double bond. The alkyl portion of an alkenyl group provided herein may be substituted as provided above. In some embodiments, the alkenyl group is a C₂₋₆ alkenyl.

The term “akynyl,” as used herein, refers to C₂₋₁₂ alkyl group, wherein at least two of the carbon atoms are sp hybridized and form a carbon-carbon triple bond between them. An alkynyl group provided herein may contain more than one carbon-carbon triple bond, but one is preferred. The alkyl portion of an alkynyl group provided herein may be substituted as provided above. In some embodiments, the alkynyl group is a C₂₋₆ alkynyl.

The terms “alkoxy,” “alkylamino,” and “alkylthio,” are used in their conventional sense, and refer to those alkyl groups attached to the remainder of the molecule via an oxygen atom (“oxy”), an amino group (“amino”) or thio group. The term “alkylamino” includes mono-di-alkylamino groups, the alkyl portions can be the same or different.

The terms “halo” or “halogen” by itself or as part of another substituent, mean, unless otherwise stated, a fluorine, chlorine, bromine, or iodine atom, but preferably fluorine or chlorine.

The term “halo(C₁₋ₓ alkyl)” refers to an alkyl that has 1-x carbon atoms and that is substituted with one or more (e.g. 1, 2, 3, 4, 5, or 6) halo groups. For example the term includes an alkyl group having 1-6 carbon atoms that is substituted with one or more halo groups. Non-limiting examples of the term halo(C₁₋₆alkyl) include fluoromethyl, difluoromethyl, trifluoromethyl, chloromethyl, and 2,2,2-trifluoroethyl.

The term “halo(C₁₋ₓ alkoxy)” refers to an alkoxy group that has 1-x carbon atoms and that is substituted with one or more (e.g. 1, 2, 3, 4, 5, or 6) halo groups. For example the term includes an alkoxy group having 1-6 carbon atoms that is substituted with one or more halo groups. Non-limiting examples halo(C₁₋₆alkyl) groups include fluoromethoxy, difluoromethoxy, trifluoromethoxy, chloromethoxy, and 2,2,2-trifluoroethoxy groups.

The term “heteroalkyl” refers to a straight- or branched-chain alkyl group, e.g. having from 2 to 14 carbons, such as 2 to 10 carbons in the chain, one or more of which has been replaced by a heteroatom selected from S, O, P, and N. Exemplary heteroalkyls include alkyl ethers, secondary and tertiary alkyl amines, alkyl amides, alkyl sulfides, and the like. The group may be a terminal group or a bridging group. As used herein reference to the normal chain when used in the context of a bridging group refers to the direct chain of atoms linking the two terminal positions of the bridging group.

The term “aryl” as used herein refers to a single all carbon aromatic ring or a multiple condensed all carbon ring system wherein at least one of the rings is aromatic. For example, in certain embodiments, an aryl group has 6 to 12 carbon atoms. Aryl includes a phenyl radical. Aryl also includes multiple condensed ring systems (e.g., ring systems comprising 2, 3 or 4 rings) having about 9 to 12 carbon atoms in which at least one ring is aromatic and wherein the other rings may be aromatic or not aromatic. Such multiple condensed ring systems are optionally substituted with one or more (e.g., 1, 2, or 3) oxo groups on any carbocycle portion of the multiple condensed ring system. The rings of the multiple condensed ring system can be connected to each other via fused, spiro and bridged bonds when allowed by valency requirements. It is to be understood that the point of attachment of a multiple condensed ring system, as defined above, can be at any position of the ring system including an aromatic or a carbocycle portion of the ring. Non-limiting examples of aryl groups include, but are not limited to, phenyl, indenyl, naphthyl, 1-, 2-, 3-, 4-tetrahydronaphthyl, and the like.

The term “heteroaryl,” as used herein, refers to a single aromatic ring that has at least one atom other than carbon in the ring, wherein the atom is selected from the group consisting of oxygen, nitrogen and sulfur; “heteroaryl” also includes multiple condensed ring systems that have at least one such aromatic ring, which multiple condensed ring systems are further described below. Thus, “heteroaryl” includes single aromatic rings of from about 1 to 6 carbon atoms and about 1-4 heteroatoms selected from the group consisting of oxygen, nitrogen and sulfur. The sulfur and nitrogen atoms may also be present in an oxidized form provided the ring is aromatic. Exemplary heteroaryl ring systems include but are not limited to pyridyl, pyrimidinyl, oxazolyl or furyl. “Heteroaryl” also includes multiple condensed ring systems (e.g., ring systems comprising 2, 3, or 4 rings) wherein a heteroaryl group, as defined above, is condensed with one or more rings selected from heteroaryls (to form for example a naphthyridinyl such as 1,8-naphthyridinyl), heterocycles, (to form for example a 1, 2, 3, 4-tetrahydronaphthyridinyl such as 1,2,3,4-tetrahydro-1,8-naphthyridinyl), carbocycles (to form for example 5,6,7,8-tetrahydroquinolyl) and aryls (to form for example indazolyl) to form the multiple condensed ring system. Thus, a heteroaryl (a single aromatic ring or multiple condensed ring system) has about 1-20 carbon atoms and about 1-6 heteroatoms within the heteroaryl ring. A heteroaryl (a single aromatic ring or multiple condensed ring system) can also have about 5 to 12 or about 5 to 10 members within the heteroaryl ring. Multiple condensed ring systems may be optionally substituted with one or more (e.g., 1, 2, 3 or 4) oxo groups on the carbocycle or heterocycle portions of the condensed ring. The rings of a multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a heteroaryl) can be at any position of the multiple condensed ring system including a heteroaryl, heterocycle, aryl or carbocycle portion of the multiple condensed ring system. It is also to be understood that the point of attachment for a heteroaryl or heteroaryl multiple condensed ring system can be at any suitable atom of the heteroaryl or heteroaryl multiple condensed ring system including a carbon atom and a heteroatom (e.g., a nitrogen). Exemplary heteroaryls include, but are not limited to pyridyl, pyrrolyl, pyrazinyl, pyrimidinyl, pyridazinyl, pyrazolyl, thienyl, indolyl, imidazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, quinazolyl, 5,6,7,8-tetrahydroisoquinolinyl benzofuranyl, benzimidazolyl, thianaphthenyl, pyrrolo[2,3-b]pyridinyl, quinazolinyl-4(3H)-one, triazolyl, 4,5,6,7-tetrahydro-1H-indazole and 3b, 4,4a, 5-tetrahydro-1H-cyclopropa[3,4]cyclo-penta[1,2-c]pyrazole. In one embodiment the term “heteroaryl” refers to a single aromatic ring containing at least one heteroatom. For example, the term includes 5-membered and 6-membered monocyclic aromatic rings that include one or more heteroatoms. Non-limiting examples of heteroaryl include but are not limited to pyridyl, furyl, thiazole, pyrimidine, oxazole, and thiadiazole.

The terms “heterocyclyl” or “heterocycle,” as used herein, refer to a single saturated or partially unsaturated ring that has at least one atom other than carbon in the ring, wherein the atom is selected from the group consisting of oxygen, nitrogen and sulfur; the term also includes multiple condensed ring systems that have at least one such saturated or partially unsaturated ring, which multiple condensed ring systems are further described below. Thus, the term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The ring may be substituted with one or more (e.g., 1, 2, or 3) oxo groups and the sulfur and nitrogen atoms may also be present in their oxidized forms. Exemplary heterocycles include but are not limited to azetidinyl, tetrahydrofuranyl and piperidinyl. The term “heterocycle” also includes multiple condensed ring systems (e.g., ring systems comprising 2, 3 or 4 rings) wherein a single heterocycle ring (as defined above) can be condensed with one or more groups selected from heterocycles (to form for example a 1,8-decahydronapthyridinyl), carbocycles (to form for example a decahydroquinolyl) and aryls to form the multiple condensed ring system. Thus, a heterocycle (a single saturated or single partially unsaturated ring or multiple condensed ring system) has about 2-20 carbon atoms and 1-6 heteroatoms within the heterocycle ring. Such multiple condensed ring systems may be optionally substituted with one or more (e.g., 1, 2, 3, or 4) oxo groups on the carbocycle or heterocycle portions of the multiple condensed ring. The rings of the multiple condensed ring system can be connected to each other via fused, spiro and bridged bonds when allowed by valency requirements. It is to be understood that the individual rings of the multiple condensed ring system may be connected in any order relative to one another. Accordingly, a heterocycle (a single saturated or single partially unsaturated ring or multiple condensed ring system) has about 3-20 atoms including about 1-6 heteroatoms within the heterocycle ring system. It is also to be understood that the point of attachment of a multiple condensed ring system (as defined above for a heterocycle) can be at any position of the multiple condensed ring system including a heterocycle, aryl and carbocycle portion of the ring. It is also to be understood that the point of attachment for a heterocycle or heterocycle multiple condensed ring system can be at any suitable atom of the heterocycle or heterocycle multiple condensed ring system including a carbon atom and a heteroatom (e.g., a nitrogen). In one embodiment the term heterocycle includes a C₂₋₂₀ heterocycle. In one embodiment the term heterocycle includes a C₂₋₇ heterocycle. In one embodiment the term heterocycle includes a C₂₋₅ heterocycle. In one embodiment the term heterocycle includes a C₂₋₄ heterocycle. Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydroquinolyl, benzoxazinyl, dihydrooxazolyl, chromanyl, 1,2-dihydropyridinyl, 2,3-dihydrobenzofuranyl, 1,3-benzodioxolyl, 1,4-benzodioxanyl, spiro[cyclopropane-1,1′-isoindolinyl]-3′-one, isoindolinyl-1-one, 2-oxa-6-azaspiro[3.3]heptanyl, imidazolidin-2-one N-methylpiperidine, imidazolidine, pyrazolidine, butyrolactam, valerolactam, imidazolidinone, hydantoin, dioxolane, phthalimide, 1,4-dioxane, thiomorpholine, thiomorpholine-S-oxide, thiomorpholine-S,S-oxide, pyran, 3-pyrroline, thiopyran, pyrone, tetrhydrothiophene, quinuclidine, tropane, 2-azaspiro[3.3]heptane, (1R,5S)-3-azabicyclo[3.2.1]octane, (1s, 4s)-2-azabicyclo[2.2.2]octane, (1R,4R)-2-oxa-5-azabicyclo[2.2.2]octane and pyrrolidin-2-one. In one embodiment the term “heterocycle” refers to a monocyclic, saturated or partially unsaturated, 3-8 membered ring having at least one heteroatom. For example, the term includes a monocyclic, saturated or partially unsaturated, 4, 5, 6, or 7 membered ring having at least one heteroatom. Non-limiting examples of heterocycle include aziridine, azetidine, pyrrolidine, piperidine, piperidine, piperazine, oxirane, morpholine, and thiomorpholine. The term “9- or 10-membered heterobicycle” as used herein refers to a partially unsaturated or aromatic fused bicyclic ring system having at least one heteroatom. For example, the term 9- or 10-membered heterobicycle includes a bicyclic ring system having a benzo ring fused to a 5-membered or 6-membered saturated, partially unsaturated, or aromatic ring that contains one or more heteroatoms.

As used herein, the term “heteroatom” is meant to include oxygen (O), nitrogen (N), sulfur (S) and silicon (Si). The nitrogen and sulfur can be in an oxidized form when feasible.

As used herein, the term “chiral” refers to molecules that have the property of non-superimposability of the mirror image partner, while the term “achiral” refers to molecules which are superimposable on their mirror image partner.

As used herein, the term “stereoisomers” refers to compounds which have identical chemical constitution, but differ with regard to the arrangement of the atoms or groups in space. As used herein a crossed line “ [symbol] ” indicates a mixture of E and Z stereoisomers.

As used herein a wavy line “ [symbol] ” or a dashed line “ [symbol] ” that intersects a bond in a chemical structure indicates the point of attachment of the bond that the wavy bond intersects in the chemical structure to the remainder of a molecule.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers can separate under high resolution analytical procedures such as electrophoresis and chromatography. “Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention can contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereoselection or stereospecificity in a chemical reaction or process. The terms “racemic mixture” and “racemate” refer to an equimolar mixture of two enantiomeric species, devoid of optical activity.

When a bond in a compound formula herein is drawn in a non-stereochemical manner (e.g., flat), the atom to which the bond is attached includes all stereochemical possibilities. When a bond in a compound formula herein is drawn in a defined stereochemical manner (e.g., bold, bold-wedge, dashed or dashed-wedge), it is to be understood that the atom to which the stereochemical bond is attached is enriched in the absolute stereoisomer depicted unless otherwise noted. In one embodiment, the compound may be at least 51% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95% the absolute stereoisomer depicted. In another embodiment, the compound may be at least 97% the absolute stereoisomer depicted. In another embodiment, the compound may be at least 98% the absolute stereoisomer depicted. In another embodiment, the compound may be at least 99% the absolute stereoisomer depicted.

As used herein, the term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

As used herein, the term “solvate” refers to an association or complex of one or more solvent molecules and a compound of the invention. Examples of solvents that form solvates include, but are not limited to, water, isopropanol, ethanol, methanol, DMSO, ethyl acetate, acetic acid, and ethanolamine. The term “hydrate” refers to the complex where the solvent molecule is water.

As used herein, the term “protecting group” refers to a substituent that is commonly employed to block or protect a particular functional group on a compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyloxycarbonyl (CBZ) and 9-fluorenylmethylenoxycarbonyl (Fmoc). Similarly, a “hydroxy-protecting group” refers to a substituent of a hydroxy group that blocks or protects the hydroxy functionality. Suitable protecting groups include acetyl and silyl. A “carboxy-protecting group” refers to a substituent of the carboxy group that blocks or protects the carboxy functionality. Common carboxy-protecting groups include phenylsulfonylethyl, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see P. G. M. Wuts and T. W. Greene, Greene's Protective Groups in Organic Synthesis 4^{th} edition, Wiley-Interscience, New York, 2006.

As used herein, the term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al. “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds can be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. As used herein the term “prodrug” refers to those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Prodrugs of the invention include compounds wherein an amino acid residue, or a polypeptide chain of two or more (e.g., two, three or four) amino acid residues, is covalently joined through an amide or ester bond to a free amino, hydroxy or carboxylic acid group of a compound of the present invention. The amino acid residues include but are not limited to the 20 naturally occurring amino acids commonly designated by three letter symbols and also includes phosphoserine, phosphothreonine, phosphotyrosine, 4-hydroxyproline, hydroxylysine, demosine, isodemosine, gamma-carboxyglutamate, hippuric acid, octahydroindole-2-carboxylic acid, statine, 1,2,3,4-tetrahydroisoquinoline-3-carboxylic acid, penicillamine, ornithine, 3-methylhistidine, norvaline, beta-alanine, gamma-aminobutyric acid, citrulline, homocysteine, homoserine, methyl-alanine, para-benzoylphenylalanine, phenylglycine, propargylglycine, sarcosine, methionine sulfone and tert-butylglycine. Additional types of prodrugs are also encompassed. For instance, a free carboxyl group of a compound of the invention can be derivatized as an amide or alkyl ester. As another example, compounds of this invention comprising free hydroxy groups can be derivatized as prodrugs by converting the hydroxy group into a group such as, but not limited to, a phosphate ester, hemisuccinate, dimethylaminoacetate, or phosphoryloxymethyloxycarbonyl group, as outlined in Fleisher, D. et al., (1996) Improved oral drug delivery: solubility limitations overcome by the use of prodrugs Advanced Drug Delivery Reviews, 19:115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers, wherein the acyl group can be an alkyl ester optionally substituted with groups including, but not limited to, ether, amine and carboxylic acid functionalities, or where the acyl group is an amino acid ester as described above, are also encompassed. Prodrugs of this type are described in J. Med. Chem., (1996), 39:10. More specific examples include replacement of the hydrogen atom of the alcohol group with a group such as (C₁₋₆)alkanoyloxymethyl, 1-((C₁₋₆)alkanoyloxy)ethyl, 1-methyl-1-((C₁₋₆)alkanoyloxy)ethyl, (C₁₋₆)alkoxycarbonyloxymethyl, N—(C₁₋₆)alkoxycarbonylaminomethyl, succinoyl, (C₁₋₆)alkanoyl, alpha-amino(C₁₋₄)alkanoyl, arylacyl and alpha-aminoacyl, or alpha-aminoacyl-alpha-aminoacyl, where each alpha-aminoacyl group is independently selected from the naturally occurring L-amino acids, P(O)(OH)₂, —P(O)(O(C₁₋₆)alkyl)₂ or glycosyl (the radical resulting from the removal of a hydroxyl group of the hemiacetal form of a carbohydrate). For additional examples of prodrug derivatives, see, for example, a) Design of Prodrugs, edited by H. Bundgaard, (Elsevier, 1985) and Methods in Enzymology, Vol. 42, p. 309-396, edited by K. Widder, et al. (Academic Press, 1985); b) A Textbook of Drug Design and Development, edited by Krogsgaard-Larsen and H. Bundgaard, Chapter 5 “Design and Application of Prodrugs,” by H. Bundgaard p. 113-191 (1991); c) H. Bundgaard, Advanced Drug Delivery Reviews, 8:1-38 (1992); d) H. Bundgaard, et al., Journal of Pharmaceutical Sciences, 77:285 (1988); and e) N. Kakeya, et al., Chem. Pharm. Bull., 32:692 (1984), each of which is specifically incorporated herein by reference.

Additionally, the present invention provides for metabolites of compounds of the invention. As used herein, a “metabolite” refers to a product produced through metabolism in the body of a specified compound or salt thereof. Such products can result for example from the oxidation, reduction, hydrolysis, amidation, deamidation, esterification, deesterification, enzymatic cleavage, and the like, of the administered compound. Metabolite products typically are identified by preparing a radiolabelled (e.g., ¹⁴C or ³H) isotope of a compound of the invention, administering it parenterally in a detectable dose (e.g., greater than about 0.5 mg/kg) to an animal such as rat, mouse, guinea pig, monkey, or to man, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g., by MS, LC/MS or NMR analysis. In general, analysis of metabolites is done in the same way as conventional drug metabolism studies well known to those skilled in the art. The metabolite products, so long as they are not otherwise found in vivo, are useful in diagnostic assays for therapeutic dosing of the compounds of the invention.

The term “patient” or “subject” is used throughout the specification to describe an animal, preferably a human or a domesticated animal, to whom treatment, including prophylactic treatment, with the compositions according to the present disclosure is provided. For treatment of those infections, conditions or disease states which are specific for a specific animal such as a human patient, the term patient refers to that specific animal, including a domesticated animal such as a dog or cat or a farm animal such as a horse, cow, sheep, etc. In general, in the present disclosure, the term patient refers to a human patient unless otherwise stated or implied from the context of the use of the term.

The term “effective” is used to describe an amount of a compound, composition or component which, when used within the context of its intended use, effects an intended result. The term effective subsumes all other effective amount or effective concentration terms, which are otherwise described or used in the present application.

### Compounds

Disclosed herein are bifunctional compounds that function to recruit endogenous proteins to an E3 ubiquitin ligase for degradation and methods of using the same. In particular, disclosed herein are bifunctional compounds that are modulators of targeted ubiquitination of a variety of polypeptides and other proteins, which then are degraded and/or otherwise inhibited by the bifunctional compounds. The bifunctional molecules of the present disclosure actively degrade SMARCA2, leading to robust cellular proliferation suppression and apoptosis induction. Bifunctional compound mediated protein degradation provides a promising strategy in targeting the pathological proteins “undruggable” by traditional approaches.

Other bifunctional that are modulators of targeted ubiquitination are disclosed in U.S. Nonprovisional patent application Ser. No. 16/590,329, filed 1 Oct. 2020, published as U.S. Patent Application Publication No. 2020/0038378 A1; U.S. Nonprovisional application Ser. No. 16/372,345, filed 1 Apr. 2019, published as U.S. Patent Application Publication No. 2019/0300521 A1; U.S. Provisional Patent Application Ser. No. 62/651,186, filed: 1 Apr. 2018, titled BRM TARGETING PROTAC COMPOUNDS AND ASSOCIATED METHODS OF USE; and U.S. Provisional Patent Application Ser. No. 62/797,754, filed: 28 Jan. 2019, titled BRM TARGETING PROTAC COMPOUNDS AND ASSOCIATED METHODS OF USE; U.S. patent application Ser. No. 15/230,354, filed on Aug. 5, 2016; and U.S. patent application Ser. No. 14/371,956, filed on Jul. 11, 2014, published as U.S. Patent Application Publication No. 2014/0356322; and U.S. patent application Ser. No. 15/074,820, filed on Mar. 18, 2016, published as U.S. Patent Application Publication No. 2016/0272639; and International Patent Application No. PCT/US2016/019328, filed Feb. 24, 2016, published as International Patent Application Publication No. WO2016/138114, and International Patent Application No. PCT/US2016/023258, filed Mar. 18, 2016, published as International Patent Application Publication No. WO2016/149668, and U.S. Non-Provisional patent application Ser. No. 15/885,671, filed 31 Jan. 2018, published as U.S. Patent Application Publication No. 2018/0215731, all of which are incorporated herein by reference in their entirety.

The disclosed bifunctional compounds provide a broad range of advantageous pharmacological activity that are consistent with the degradation/inhibition of targeted polypeptides from a multitude of different protein classes and/or families.

In any aspect or embodiment described herein, the disclosed bifunctional compounds comprise an E3 ubiquitin ligase binding moiety (“ULM”) that is a Von Hippel-Lindae E3 ubiquitin ligase (VHL) binding moiety (VLM). In an exemplary embodiment, the ULM is coupled to a target protein binding moiety (PTM) via a chemical linker (L) according to the structure:

PTM-L-ULM

wherein L is a bond or a chemical linker group, ULM is a E3 ubiquitin ligase binding moiety, and PTM is a target protein binding moiety. The number and/or relative positions of the moieties in the compounds illustrated herein is provided by way of example only. As would be understood by the skilled artisan, compounds described herein can be synthesized with any desired number and/or relative position of the respective functional moieties.

In another aspect, the present disclosure provides bifunctional or multifunctional compounds (e.g., PROTACs) useful for regulating protein activity by inducing the degradation of a target protein. In certain embodiments, the compound comprises a VLM coupled, e.g., linked covalently, directly or indirectly, to a moiety that binds a target protein (i.e., a protein targeting moiety or a “PTM”). In certain embodiments, the VLM and PTM are joined or coupled via a chemical linker (L). The VLM binds VHL, and the PTM recognizes a target protein and the interaction of the respective moieties with their targets facilitates the degradation of the target protein by placing the target protein in proximity to the ubiquitin ligase protein. An exemplary bifunctional compound can be depicted as:

PTM-VLM.

In certain embodiments, the bifunctional compound further comprises a chemical linker (“L”). For example, the bifunctional compound can be depicted as:

PTM-L-VLM,

wherein the PTM is a protein/polypeptide targeting moiety, the L is a chemical linker, and the VLM is a VHL binding moiety.

In any aspect or embodiment described herein, the description provides the following exemplary SMARCA2 (i.e., BRAHMA or BRM) heterobirunctional degradative compounds (compounds 1-157 of Table 1), including pharmaceutically acceptable salts thereof. In any aspect or embodiment described herein, the description provides bifunctional compounds having the chemical structure: PTM-L-ULM, or a pharmaceutically acceptable salt thereof, wherein: the ULM is a small molecule E3 ubiquitin ligase binding moiety that binds a Von Hippel-Lindau E3 ubiquitin ligase as described in any aspect or embodiment described herein; the L is a bond or a chemical linking moiety connecting the ULM and the PTM as described in any aspect or embodiment described herein; and the PTM is a small molecule comprising a SMARCA2 protein targeting moiety as described in any aspect or embodiment described herein.

In any aspect or embodiment described herein, the ULM (e.g., VLM) shows activity or binds to the E3 ubiquitin ligase (e.g., VHL) with an IC₅₀ of less than about 200 μM. The IC₅₀ can be determined according to any method known in the art (e.g., a fluorescent polarization assay).

The IC₅₀ values of the bifunctional compounds described herein can be determined according to any method known in the art such as, for example, a fluorescent polarization assay.

In any aspect or embodiment described herein, the ULM (e.g., VLM) shows activity or binds to the E3 ubiquitin ligase (e.g., VHL) with an IC₅₀ of less than about 200 μM. For example, in any aspect or embodiment described herein, the bifunctional compounds described herein demonstrate an activity with an IC₅₀ of less than about 100 mM, less than about 50 mM, less than about 10 mM, less than about 1 mM, less than about 0.5 mM, less than about 0.1 mM, less than about 0.05 mM, less than about 0.01 mM, less than about 0.005 mM, or less than about 0.001 mM.

In any aspect or embodiment described herein, the bifunctional compounds described herein demonstrate an activity with an IC₅₀ of less than about 100 PM, less than about 50 μM, less than about 10 μM, less than about 1 μM, less than about 0.5 μM, less than about 0.1 μM, less than about 0.05 μM, less than about 0.01 μM, less than about 0.005 μM, or less than about 0.001 μM.

In any aspect or embodiment described herein, the bifunctional compounds described herein demonstrate an activity with an IC₅₀ of less than about 100 nM, less than about 50 nM, less than about 10 nM, less than about 1 nM, less than about 0.5 nM, less than about 0.1 nM, less than about 0.05 nM, less than about 0.01 nM, less than about 0.005 nM, less than about 0.001 nM.

In any aspect or embodiment described herein, the bifunctional compounds described herein demonstrate an activity with an IC₅₀ of less than about 100 μM, less than about 50 μM, less than about 10 μM, less than about 1 μM, less than about 0.5 μM, less than about 0.1 μM, less than about 0.05 μM, less than about 0.01 μM, less than about 0.005 μM, or less than about 0.001 μM.

In any aspect or embodiment described herein, the Dₘₐₓ of the bifunctional compounds described herein can be determined according to any method known in the art such as, for example, a fluorescent polarization assay.

In any aspect or embodiment described herein, the bifunctional compounds have a Dₘₐₓ greater than or equal to 80%.

In any aspect or embodiment described herein, the bifunctional compounds have a Dₘₐₓ greater than 50%, greater than 75%, or greater than or equal to 80%. In any aspect or embodiment described herein, the bifunctional compounds have a Dₘₐₓ greater than 50%. In any aspect or embodiment described herein, the bifunctional compounds have a Dₘₐₓ greater than 75%.

In any aspect or embodiment descried herein, the DC₅₀ value of the bifunctional compounds described herein can be determined according to any method known in the art such as, for example, a fluorescent polarization assay.

In any aspect or embodiment described herein, DC₅₀ value of the bifunctional compounds is less than 10 nM or less than 2.5 nM. In any aspect or embodiment described herein, DC₅₀ value of the bifunctional compounds is less than 10 nM. In any aspect or embodiment described herein, DC₅₀ value of the bifunctional compounds is less than 2.5 nM.

In any aspect or embodiment described herein, the bifunctional compounds have a Dₘₐₓ greater than 50%, greater than 75%, or greater than or equal to 80% and DC₅₀ value of the bifunctional compounds is less than 10 nM or less than 2.5 nM.

In any aspect or embodiment described herein, the bifunctional compound includes compounds having a DC₅₀ of <about 2.5 nM (i.e., category A as described herein), wherein the DC₅₀ is optionally determined as described herein.

In any aspect or embodiment described herein, the bifunctional compound includes compounds having a DC₅₀ that is ≥about 2.5 nM and <about 10 nM (i.e., category B as described herein), wherein the DC₅₀ is optionally determined as described herein.

In any aspect or embodiment described herein, the bifunctional compound includes compounds having a DC₅₀ of ≥about 2.5 nM and <about 30 nM (i.e., category C as described herein), wherein the DC₅₀ is optionally determined as described herein.

In any aspect or embodiment described herein, the bifunctional compound includes compounds having a DC₅₀ of ≥about 30 nM (i.e., category D as described herein), wherein the DC₅₀ is optionally determined as described herein.

In any aspect or embodiment described herein, a compound or compounds having a DC₅₀ of ≥about 30 nM (i.e., category D as described herein) is or are excluded (optionally, the DC₅₀ can be determined as described herein).

In any aspect or embodiment described herein the DC₅₀ value of the bifunctional compounds described herein can be determined according to any method know in the art, such as, for example, a fluorescent polarization assay or as described herein.

In any aspect or embodiment described herein, the bifunctional compound includes compounds having a D_{Max} of >about 75% degraded (i.e., category A as described herein), wherein the D_{Max} is optionally determined as described herein.

In any aspect or embodiment described herein, the bifunctional compound includes compounds having a D_{Max} that is >about 50% degraded and about 75% degraded (i.e., category B as described herein), wherein the DC₅₀ is optionally determined as described herein.

In any aspect or embodiment described herein, the bifunctional compound includes compounds having a D_{Max} of ≤about 50% degraded (i.e., category C as described herein), wherein the D_{Max} is optionally determined as described herein.

In any aspect or embodiment described herein a compound or compounds having a D_{Max} of ≤about 50% degraded (i.e., category C as described herein) is or are excluded (optionally, the D_{Max} can be determined as described herein).

In any aspect or embodiment described herein the D_{Max} value of the bifunctional compounds described herein can be determined according to any method know in the art, such as, for example, a fluorescent polarization assay or as described herein.

In any aspect or embodiment described herein, where the compound comprises multiple ULMs, the ULMs are identical. In any aspect or embodiment described herein, the compound comprising a plurality of ULMs (e.g., ULM, etc.), at least one PTM coupled to a ULM directly or via a chemical linker (L) or both. In any aspect or embodiment described herein, the compound comprising a plurality of ULMs further comprises multiple PTMs. In any aspect or embodiment described herein, the PTMs are the same or, optionally, different. In any aspect or embodiment described herein, wherein the PTMs are different, the respective PTMs may bind the same protein target or bind specifically to a different protein target.

In any aspect or embodiment described herein, the compound may comprise a plurality of ULMs. In any aspect or embodiment described herein, the compound comprising at least two different ULMs and/or a plurality of ULMs further comprises at least one PTM coupled to a ULM directly or via a chemical linker or both. In any aspect or embodiment described herein, a compound comprising at least two different ULMs can further comprise multiple PTMs. In any aspect or embodiment described herein, the PTMs are the same or, optionally, different. In any aspect or embodiment described herein, wherein the PTMs are different the respective PTMs may bind the same protein target or bind specifically to a different protein target.

In any aspect or embodiment described herein, the compound has a chemical structure selected from:

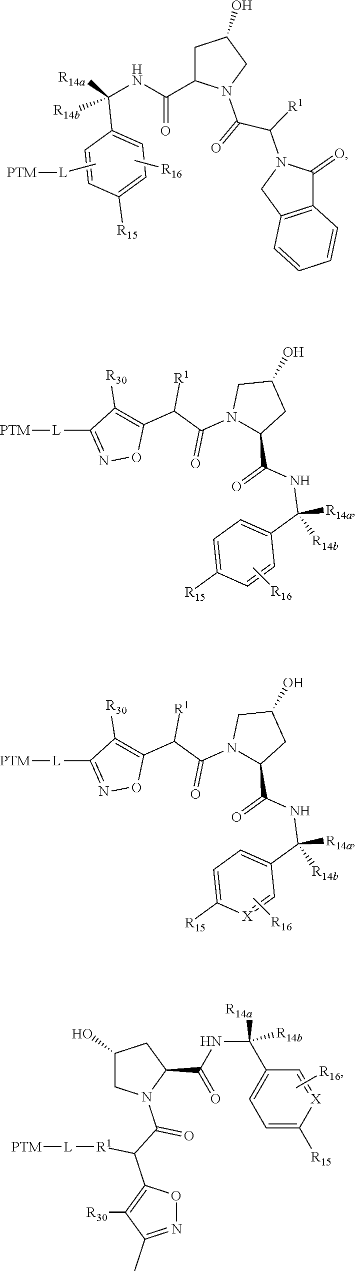

or a pharmaceutically acceptable salt thereof, wherein PTM, L, X, R₃₀, R₁, R_{28A}, R_{28B}, R₂₈, R₁₄ₐ, R_{14b}, R₁₅, and R₁₆ are as defined in any aspect or embodiment described herein, including different variable names found at the same location within the chemical structure.

In any aspect or embodiment described herein, the compound has a chemical structure selected from:

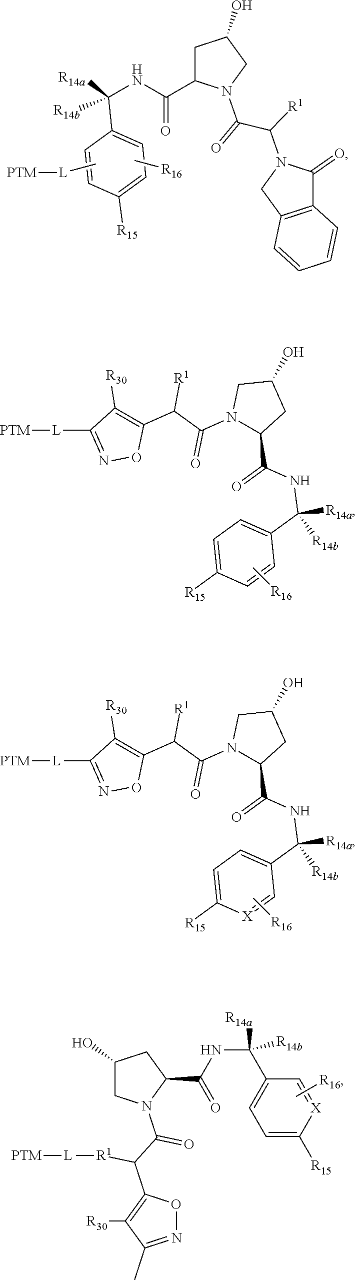

or a pharmaceutically acceptable salt thereof, wherein:
PTM and L are as defined in any aspect or embodiment described herein;
R₁₄ₐ, R_{14b}, R₁₅, and R₁₆ are as defined in any aspect or embodiment described herein, including different variable names found at the same location within the chemical structure;
X is CH or N;
R₃₀ is H, F, or Cl;
R₁ is a C₁₋₆ alkyl;
R_{28A} is selected from H or methyl;
R_{28B} is selected from H, methyl, and halogen (e.g., F or Cl); and
R₂₈ is H, methyl, CH₂N(Me)₂, CH₂OH, CH₂O(C₁₋₄alkyl), CH₂NHC(O)C₁₋₄alkyl, NH₂,

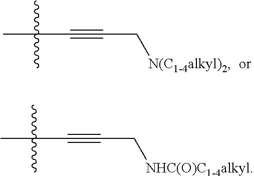

In any aspect or embodiment described herein, the compound has a chemical structure selected from:

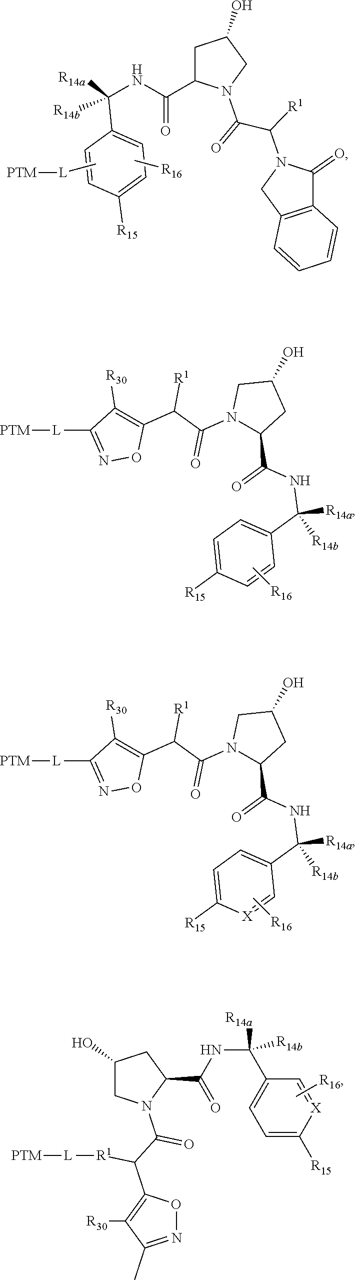

or a pharmaceutically acceptable salt thereof, wherein:
PTM and L are as defined in any aspect or embodiment described herein;
X is CH or N;
R₃₀ is H, F or Cl;
R₁ is a C₁₋₆ alkyl;
R_{28A} is selected from H or methyl;
R_{28B} is selected from H, methyl, and halogen (e.g., F or Cl);
R₂₈ is H, methyl, CH₂N(Me)₂, CH₂OH, CH₂O(C₁₋₄alkyl), CH₂NHC(O)C₁₋₄alkyl, NH₂,

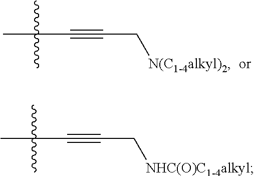

one of R₁₄ₐ and R_{14b} is a H, methyl, C1 fluoroalkyl, CHF₂, CF₃, and the other is a H;
R₁₅ is selected from: cyano, halogen (e.g., F or Cl),

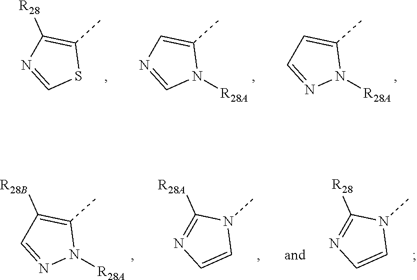

R¹⁶ is one or two groups individually selected from H, C₁₋₄alkyl, fluoro, chloro, NH₂, CN, or C₁₋₄alkoxy.

In additional embodiments, the description provides the compounds as described herein including their enantiomers, diastereomers, solvates and polymorphs, including pharmaceutically acceptable salt forms thereof, e.g., acid and base salt forms.

### Exemplary VLMs

In any aspect or embodiment described herein, the ULM has a chemical structure selected from:

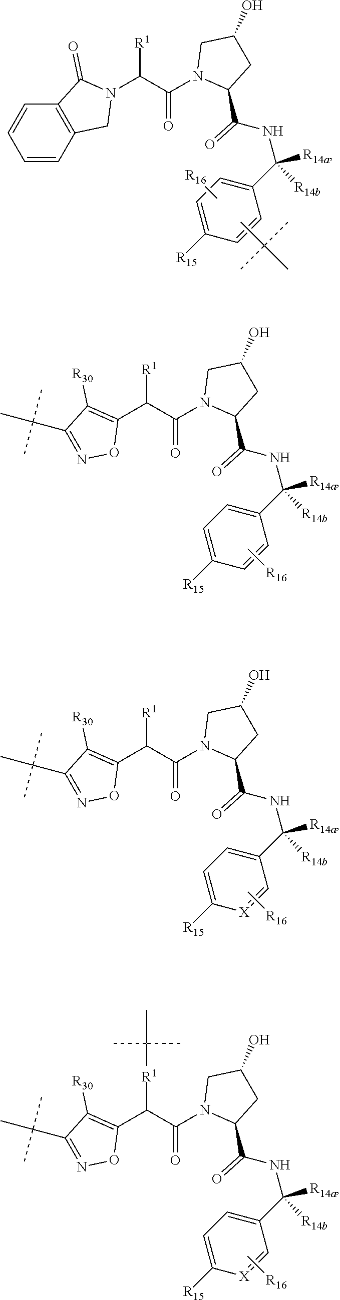

or a pharmaceutically acceptable salt thereof, wherein X, R₃₀, R₁, R_{28A}, R_{28B}, R₂₈, R₁₄ₐ, R_{14b}, R₁₅, and R₁₆ are as defined in any aspect or embodiment described herein.

In any aspect or embodiment described herein, the ULM has a chemical structure selected from:

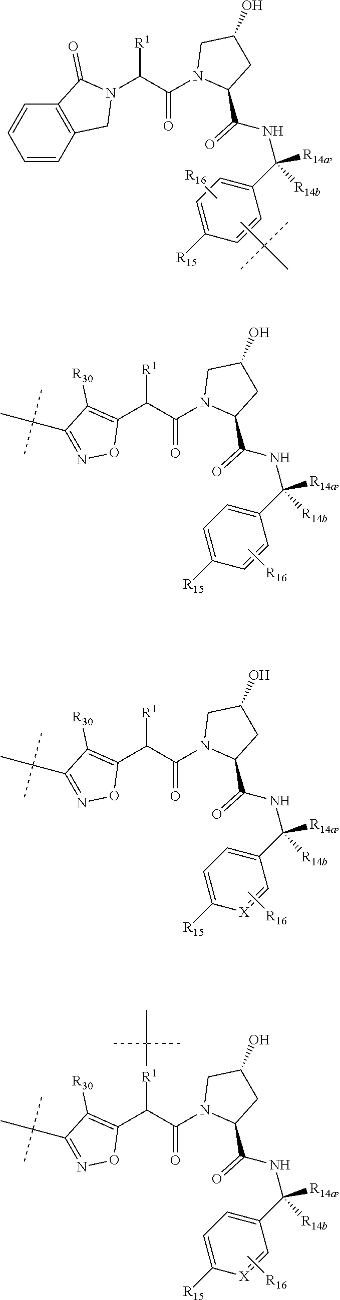

or a pharmaceutically acceptable salt thereof,
wherein:
R₁₄ₐ, R_{14b}, R₁₅, and R₁₆ are as defined in any aspect or embodiment described herein;
X is CH or N;
R₃₀ is H, F or Cl;
R₁ is a C₁₋₆ alkyl;
R_{28A} is selected from H or methyl;
R_{28B} is selected from H, methyl, and halogen (e.g., F or Cl); and
R₂₈ is H, methyl, CH₂N(Me)₂, CH₂OH, CH₂O(C₁₋₄alkyl), CH₂NHC(O)C₁₋₄alkyl, NH₂,

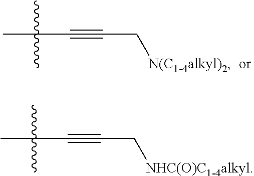

In any aspect or embodiment described herein, the ULM has a chemical structure selected from:

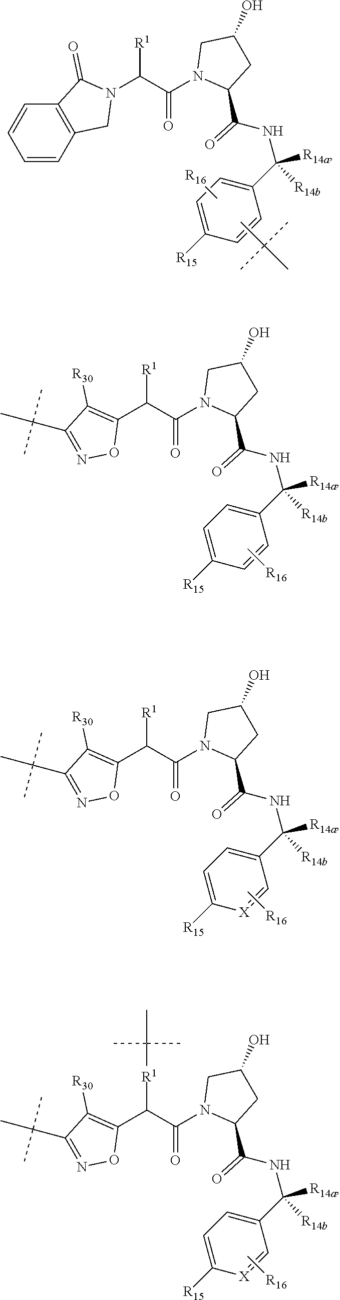

or a pharmaceutically acceptable salt thereof,
wherein:
X is CH or N;
R₃₀ is H, F or Cl;
R₁ is a C₁₋₆ alkyl;
R_{28A} is selected from H or methyl;
R_{28B} is selected from H, methyl, and halogen (e.g., F or Cl);
R₂₈ is H, methyl, CH₂N(Me)₂, CH₂OH, CH₂O(C₁₋₄alkyl), CH₂NHC(O)C₁₋₄alkyl, NH₂, N(C₁₋₄alkyl)₂ or NHC(O)C₁₋₄alkyl

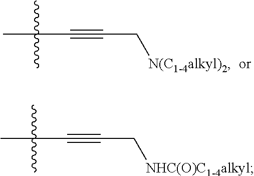

one of R₁₄ₐ and R_{14b} is a H, methyl, Cl fluoroalkyl, CHF₂, CF₃, and the other is a H;
R₁₅ is selected from: cyano, halogen (e.g., F or Cl),

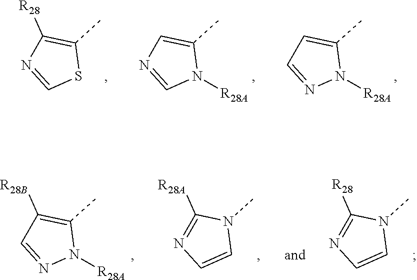

and
R¹⁶ is one or two groups individually selected from H, C₁₋₄alkyl, fluoro, chloro, NH₂, CN, or C₁₋₄alkoxy.

In any aspect or embodiment described herein, the ULM is selected from:

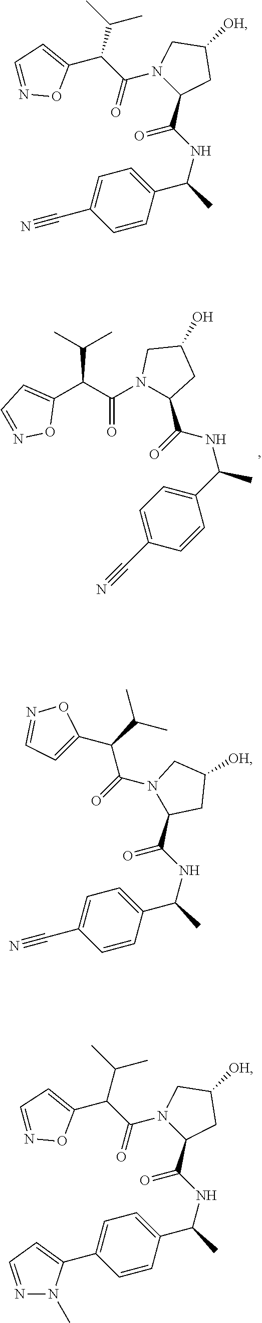 [SMILES: CC(C)[C@H](C(=O)N1C[C@H](O)C[C@H]1C(=O)N[C@@H](C)c1ccc(C#N)cc1)c1ccno1 ; CC(C)[C@@H](C(=O)N1C[C@H](O)C[C@H]1C(=O)N[C@@H](C)c1ccc(C#N)cc1)c1ccno1 ; CC(C)[C@@H](C(=O)N1C[C@H](O)C[C@H]1C(=O)N[C@@H](C)c1ccc(C#N)cc1)c1ccno1 ; CC(C)C(C(=O)N1C[C@H](O)C[C@H]1C(=O)N[C@@H](C)c1ccc(-c2ccnn2C)cc1)c1ccno1]

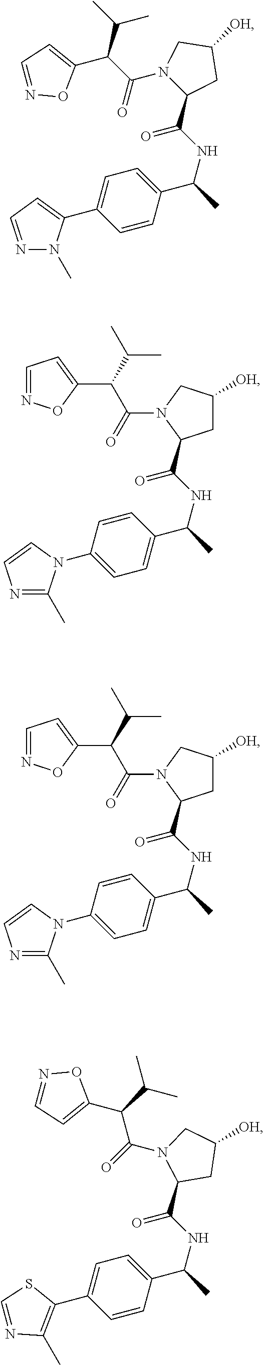 [SMILES: CC(C)[C@@H](C(=O)N1C[C@H](O)C[C@H]1C(=O)N[C@@H](C)c1ccc(-c2ccnn2C)cc1)c1ccno1 ; Cc1nccn1-c1ccc([C@H](C)NC(=O)[C@@H]2C[C@@H](O)CN2C(=O)[C@H](c2ccno2)C(C)C)cc1 ; Cc1nccn1-c1ccc([C@H](C)NC(=O)[C@@H]2C[C@@H](O)CN2C(=O)[C@@H](c2ccno2)C(C)C)cc1 ; Cc1ncsc1-c1ccc([C@H](C)NC(=O)[C@@H]2C[C@@H](O)CN2C(=O)[C@@H](c2ccno2)C(C)C)cc1]

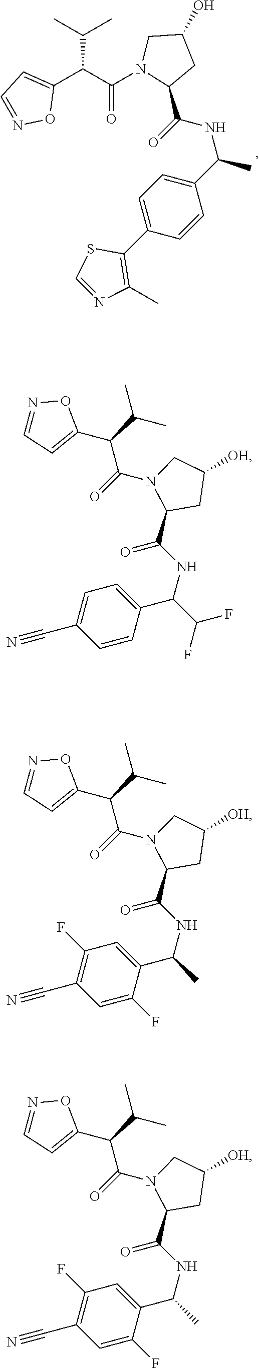 [SMILES: Cc1ncsc1-c1ccc([C@H](C)NC(=O)[C@@H]2C[C@@H](O)CN2C(=O)[C@H](c2ccno2)C(C)C)cc1 ; CC(C)[C@@H](C(=O)N1C[C@H](O)C[C@H]1C(=O)NC(c1ccc(C#N)cc1)C(F)F)c1ccno1 ; CC(C)[C@@H](C(=O)N1C[C@H](O)C[C@H]1C(=O)N[C@@H](C)c1cc(F)c(C#N)cc1F)c1ccno1 ; CC(C)[C@@H](C(=O)N1C[C@H](O)C[C@H]1C(=O)N[C@H](C)c1cc(F)c(C#N)cc1F)c1ccno1]

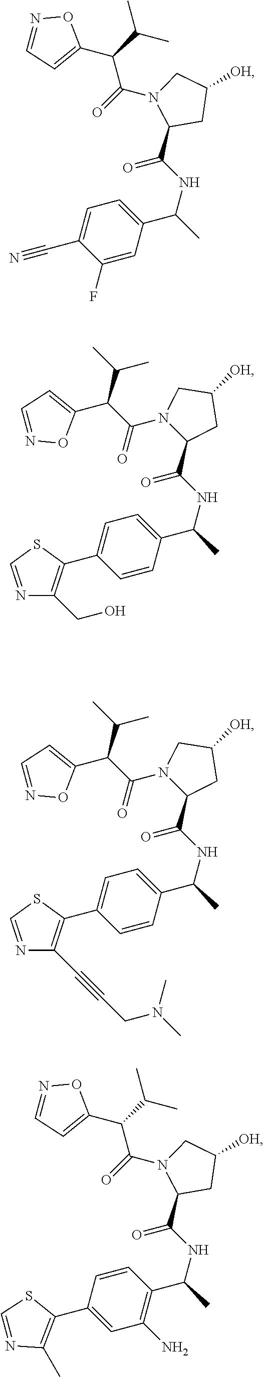 [SMILES: CC(NC(=O)[C@@H]1C[C@@H](O)CN1C(=O)[C@@H](c1ccno1)C(C)C)c1ccc(C#N)c(F)c1 ; CC(C)[C@@H](C(=O)N1C[C@H](O)C[C@H]1C(=O)N[C@@H](C)c1ccc(-c2scnc2CO)cc1)c1ccno1 ; CC(C)[C@@H](C(=O)N1C[C@H](O)C[C@H]1C(=O)N[C@@H](C)c1ccc(-c2scnc2C#CCN(C)C)cc1)c1ccno1 ; Cc1ncsc1-c1ccc([C@H](C)NC(=O)[C@@H]2C[C@@H](O)CN2C(=O)[C@H](c2ccno2)C(C)C)c(N)c1]

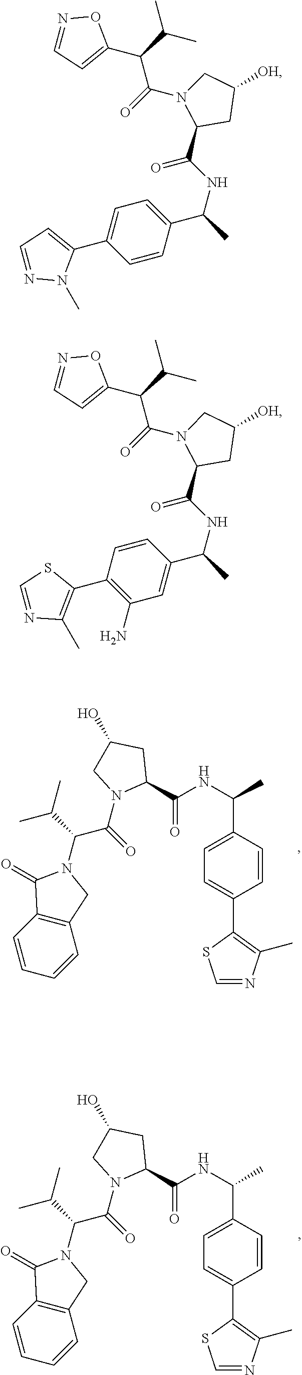 [SMILES: CC(C)[C@@H](C(=O)N1C[C@H](O)C[C@H]1C(=O)N[C@@H](C)c1ccc(-c2ccnn2C)cc1)c1ccno1 ; Cc1ncsc1-c1ccc([C@H](C)NC(=O)[C@@H]2C[C@@H](O)CN2C(=O)[C@@H](c2ccno2)C(C)C)cc1N ; Cc1ncsc1-c1ccc([C@H](C)NC(=O)[C@@H]2C[C@@H](O)CN2C(=O)[C@@H](C(C)C)N2Cc3ccccc3C2=O)cc1 ; Cc1ncsc1-c1ccc([C@@H](C)NC(=O)[C@@H]2C[C@@H](O)CN2C(=O)[C@@H](C(C)C)N2Cc3ccccc3C2=O)cc1]

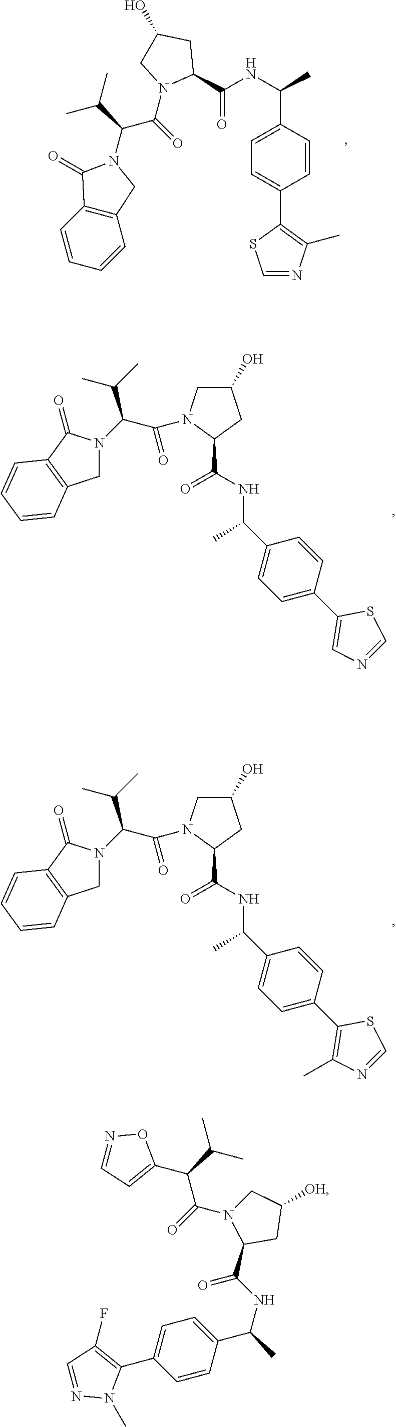 [SMILES: Cc1ncsc1-c1ccc([C@H](C)NC(=O)[C@@H]2C[C@@H](O)CN2C(=O)[C@H](C(C)C)N2Cc3ccccc3C2=O)cc1 ; CC(C)[C@@H](C(=O)N1C[C@H](O)C[C@H]1C(=O)N[C@@H](C)c1ccc(-c2cncs2)cc1)N1Cc2ccccc2C1=O ; Cc1ncsc1-c1ccc([C@H](C)NC(=O)[C@@H]2C[C@@H](O)CN2C(=O)[C@H](C(C)C)N2Cc3ccccc3C2=O)cc1 ; CC(C)[C@@H](C(=O)N1C[C@H](O)C[C@H]1C(=O)N[C@@H](C)c1ccc(-c2c(F)cnn2C)cc1)c1ccno1]

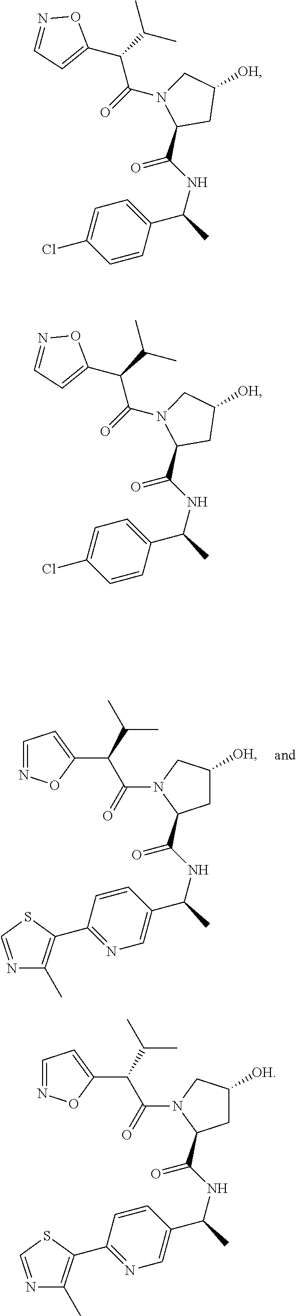 [SMILES: CC(C)[C@H](C(=O)N1C[C@H](O)C[C@H]1C(=O)N[C@@H](C)c1ccc(Cl)cc1)c1ccno1 ; CC(C)[C@@H](C(=O)N1C[C@H](O)C[C@H]1C(=O)N[C@@H](C)c1ccc(Cl)cc1)c1ccno1 ; Cc1ncsc1-c1ccc([C@H](C)NC(=O)[C@@H]2C[C@@H](O)CN2C(=O)[C@@H](c2ccno2)C(C)C)cn1 ; Cc1ncsc1-c1ccc([C@H](C)NC(=O)[C@@H]2C[C@@H](O)CN2C(=O)[C@H](c2ccno2)C(C)C)cn1]

In certain embodiments the compounds as described herein include a means for binding an E3 ubiquitin ligase, e.g., Von Hippel-Lindau E3 ubiquitin ligase. In certain embodiments the ULM is VLM and comprises a chemical structure selected from the group ULM-a:

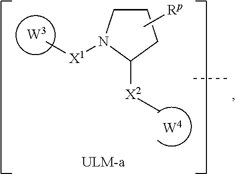

wherein:
a dashed line indicates the attachment of at least one PTM, another ULM or VLM (i.e., VLM′), or a chemical linker moiety coupling at least one PTM, or a VLM′ to the other end of the linker;
X¹, X² of Formula ULM-a are each independently selected from the group of a bond, O, NR^{Y3}, CR^{Y3}R^{Y4}, C═O, C═S, SO, and SO₂;
R^{Y3}, R^{Y4} of Formula ULM-a are each independently selected from the group of H, linear or branched C₁₋₆ alkyl, optionally substituted by 1 or more halo, optionally substituted C₁₋₆ alkoxyl (e.g., optionally substituted by 0-3 R^{P} groups);
R^{P} of Formula ULM-a is 0, 1, 2, or 3 groups, each independently selected from the group H, halo, —OH, C₁₋₃ alkyl, C═O, alkyl, alkoxy or a combination thereof;
W³ of Formula ULM-a is selected from the group of an optionally substituted T, an optionally substituted -T-N(R^{1a}R^{1b})X³, optionally substituted -T-N(R^{1a}R^{1b}) optionally substituted -T-Aryl, an optionally substituted -T-Heteroaryl, an optionally substituted T-biheteroaryl, an optionally substituted -T-Heterocyclyl, an optionally substituted -T-biheterocyclyl, an optionally substituted —NR¹-T-Aryl, an optionally substituted —NR¹-T-Heteroaryl or an optionally substituted —NR¹-T-Heterocyclyl;
X³ of Formula ULM-a is C═O, R¹, R^{1a}, R^{1b};
each of R¹, R^{1a}, R^{1b} is independently selected from the group consisting of H, linear or branched C₁₋₆ alkyl group optionally substituted by 1 or more halo or —OH groups, R^{Y3}C═O, R^{Y3}C═S, R^{Y3}SO, R^{Y3}SO₂, N(R^{Y3}R^{Y4})C═O, N(R^{Y3}R^{Y4})C═S, N(R^{Y3}R^{Y4})SO, and N(R^{Y3}R^{Y4})SO₂;
T of Formula ULM-a is selected from the group of an optionally substituted alkyl, —(CH₂)ₙ— group, —(CH₂)ₙ—O—C₁₋₆ alkyl which is optionally substituted, linear, branched, or —(CH₂)ₙ— O-heterocyclyl which is optionally substituted, wherein each one of the methylene groups is optionally substituted with one or two substituents selected from the group of halogen, methyl, a linear or branched C₁₋₆ alkyl group optionally substituted by 1 or more halogen or —OH groups, an amino acid side chain optionally substituted or an optionally substituted heterocyclyl;
W⁴ of Formula ULM-a is an optionally substituted —NR1-T-Aryl wherein the aryl group may be optionally substituted with an optionally substituted 5-6 membered heteroaryl or an optionally substituted aryl, an optionally substituted —NR1-T-Heteroaryl group, wherein the heteroaryl is optionally substituted with an optionally substituted aryl or an optionally substituted heteroaryl, or an optionally substituted —NR1-T-Heterocyclyl, where —NR1 is covalently bonded to X² and R¹ is H or CH₃, preferably H; and wherein the dashed line indicates the site of attachment of at least one PTM, or a chemical linker moiety coupling at least one PTM to ULM.

In any aspect or embodiment described herein, R^{P} is modified to form a prodrug, including by an ester or ether linkage.

In any aspect or embodiment described herein, T is selected from the group of an optionally substituted alkyl, —(CH₂)ₙ— group, wherein each one of the methylene groups is optionally substituted with one or two substituents selected from the group of halogen, methyl, optionally substituted alkoxy, a linear or branched C₁₋₆alkyl group optionally substituted by 1 or more halogen, C(O) NR¹R^{1a}, or NR¹R^{1a} or R¹ and R^{1a} are joined to form an optionally substituted heterocyclyl, or —OH groups or an amino acid side chain optionally substituted; and n is 0 to 6, often 0, 1, 2, or 3, preferably 0 or 1.

In any aspect or embodiment described herein, W⁴ of Formula ULM-a is

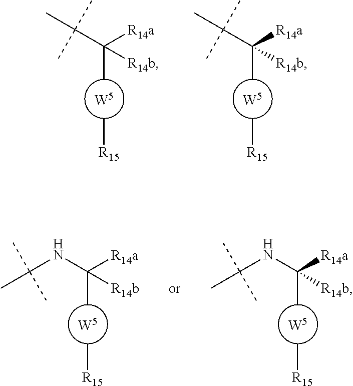

wherein R₁₄ₐ, R_{14b}, are each independently selected from the group of H, haloalkyl (e.g., fluoroalkyl), optionally substituted alkyl, optionally substituted alkoxy, optionally substituted hydroxyl alkyl, optionally substituted alkylamine, optionally substituted amide, optionally substituted alkyl-amide, optionally substituted alkyl-cyano, optionally substituted alkyl-phosphate, optionally substituted heteroalkyl, optionally substituted alkyl-heterocycloalkyl, optionally substituted alkoxy-heterocycloalkyl, COR₂₆, alkyl-COR₂₆, CONR₂₇ₐR_{27b}, NHCOR₂₆, or NHCH₃COR₂₆; and the other of R₁₄ₐ and R_{14b} is H; or R₁₄ₐ, R_{14b}, together with the carbon atom to which they are attached, form an optionally substituted 3 to 5 membered cycloalkyl, heterocycloalkyl, spirocycloalkyl or spiroheterocyclyl, wherein the spiroheterocyclyl is not epoxide or aziridine.

In any aspect or embodiment described herein, W⁵ of Formula ULM-a is selected from the group of an optionally substituted phenyl, an optionally substituted napthyl, or an optionally substituted 5-10 membered heteroaryl.

In any aspect or embodiment described herein, R₁₅ of Formula ULM-a is selected from the group of H, halogen, CN, C≡CH, OH, NO₂, NR₁₄ₐR_{14b}, OR₁₄ₐ, CONR₁₄ₐR_{14b}, NR₁₄ₐCOR_{14b}, SO₂NR₁₄ₐR_{14b}, NR₁₄ₐ SO₂R_{14b}, optionally substituted alkyl, optionally substituted haloalkyl, optionally substituted haloalkoxy, optionally substituted aryl, optionally substituted heteroaryl, optionally substituted cycloalkyl, or optionally substituted heterocyclyl.

In additional embodiments, W⁴ substituents for use in the present disclosure also include specifically (and without limitation to the specific compound disclosed) the W⁴ substituents which are found in the identified compounds disclosed herein. Each of these W⁴ substituents may be used in conjunction with any number of W³ substituents which are also disclosed herein.

In certain additional embodiments, ULM-a, is optionally substituted by 0-3 R^{P} groups in the pyrrolidine moiety. Each R^{P} is independently H, halo, —OH, C₁₋₃ alkyl, C═O.

In any of the embodiments described herein, the W³ and/or W⁴ of Formula ULM-a can independently be covalently coupled to a linker that is attached one or more PTM groups.

In certain embodiments, ULM is VHL and is represented by the structure:

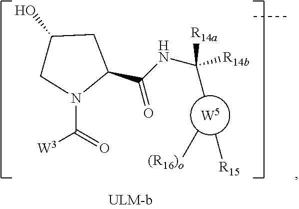

wherein:
W³ of Formula ULM-b is selected from the group of an optionally substituted aryl, optionally substituted heteroaryl, or

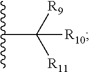

R₉ and R₁₀ of Formula ULM-b are independently hydrogen, optionally substituted alkyl, optionally substituted cycloalkyl, optionally substituted hydroxyalkyl, optionally substituted heteroaryl, or haloalkyl, or R₉, R₁₀, and the carbon atom to which they are attached form an optionally substituted cycloalkyl;
R₁₁ of Formula ULM-b is selected from the group of an optionally substituted heterocyclyl, optionally substituted alkoxy, optionally substituted heteroaryl, optionally substituted aryl,

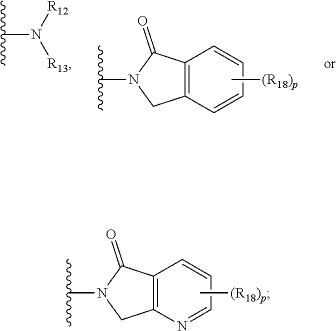

R₁₂ of Formula ULM-b is selected from the group of H or optionally substituted alkyl;
R₁₃ of Formula ULM-b is selected from the group of H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl;
R₁₄ₐ, R_{14b} of Formula ULM-b, are each independently selected from the group of H, haloalkyl (e.g. fluoroalkyl), optionally substituted alkyl, optionally substitute alkoxy, aminomethyl, alkylaminomethyl, alkoxymethyl, optionally substituted hydroxyl alkyl, optionally substituted alkylamine, optionally substituted amide, optionally substituted alkyl-amide, optionally substituted alkyl-cyano, optionally substituted alkyl-phosphate, optionally substituted heteroalkyl, optionally substituted alkyl-heterocycloalkyl, optionally substituted alkoxy-heterocycloalkyl, COR₂₆, alkyl-COR₂₆, CONR₂₇ₐR_{27b}, CH₂NHCOR₂₆, or (CH₂)N(CH₃)COR₂₆; and the other of R₁₄ₐ and R_{14b} is H; or R₁₄ₐ, R_{14b}, together with the carbon atom to which they are attached, form an optionally substituted 3 to 6 membered cycloalkyl, heterocycloalky, spirocycloalkyl or spiroheterocyclyl, wherein the spiroheterocyclyl is not epoxide or aziridine;
W⁵ of Formula ULM-b is selected from the group of a phenyl, napthyl, or a 5-10 membered heteroaryl;
R₁₅ of Formula ULM-b is selected from the group of H, halogen, CN, C≡CH, OH, NO₂, NR₂₇ₐR_{27b}, OR₂₇ₐ, CONR₂₇ₐR_{27b}, NR₂₇ₐCOR_{27b}, SO₂NR₂₇ₐR_{27b}, NR₂₇ₐ SO₂R_{27b}, optionally substituted alkyl, optionally substituted haloalkyl, optionally substituted haloalkoxy, optionally substituted aryl, optionally substituted heteroaryl, optionally substituted cycloalkyl, or optionally substituted heterocyclyl;
each R₁₆ of Formula ULM-b is independently selected from the group of halo, CN, optionally substituted alkyl, optionally substituted alkylamine, optionally substituted haloalkyl, hydroxy, or optionally substituted haloalkoxy;
o of Formula ULM-b is 0, 1, 2, 3, or 4;
R₁₈ of Formula ULM-b is independently selected from the group of H, halo, optionally substituted alkoxy, cyano, optionally substituted alkyl, haloalkyl, haloalkoxy or a linker;
each R₂₆ is independently selected from H, OH, optionally substituted alkyl or NR₂₇ₐR_{27b};
each R₂₇ₐ and R_{27b} is independently H, optionally substituted alkyl, optionally substituted 3-5 member cycloalkyl, or R₂₇ₐ and R_{27b} together with the nitrogen atom to which they are attached form a 4-6 membered heterocyclyl; and
p of Formula ULM-b is 0, 1, 2, 3, or 4,
wherein the dashed line indicates the site of attachment of at least one PTM, another ULM or a chemical linker moiety coupling at least one PTM or both to ULM.

In certain embodiments, R₁₅ of Formula ULM-b

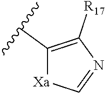

wherein R₁₇ is H, halo, optionally substituted C₃₋₆cycloalkyl, optionally substituted C₁₋₆alkyl, optionally substituted C₁₋₆alkenyl, and C₁₋₆haloalkyl; and Xa is S or O.

In certain embodiments, R₁₇ of Formula ULM-b is selected from the group methyl, ethyl, isopropyl, and cyclopropyl.

In certain additional embodiments, R₁₅ of Formula ULM-b is selected from:

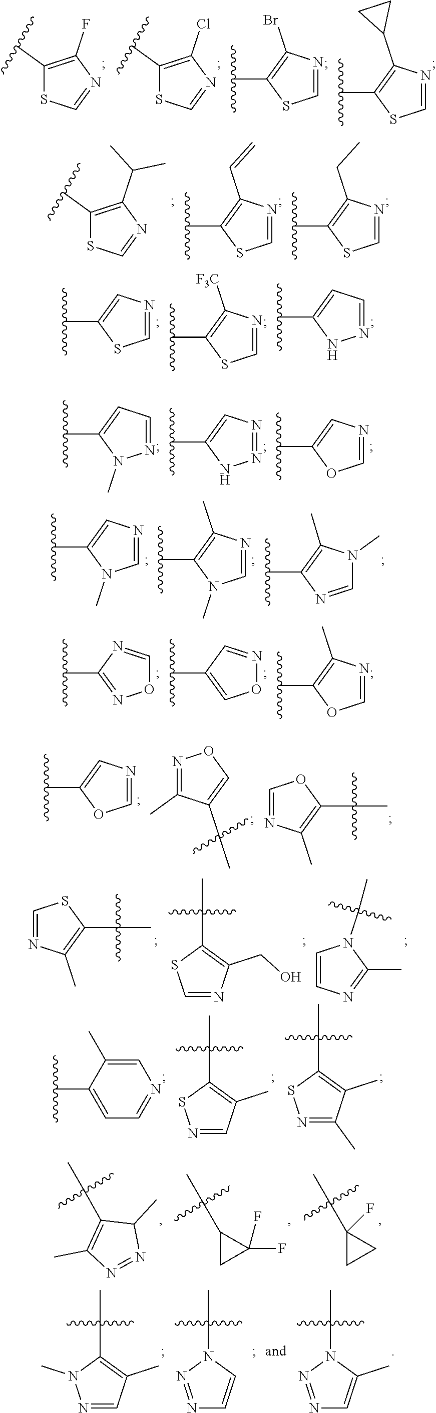

In certain embodiments, R₁₁ of Formula ULM-b is selected from:

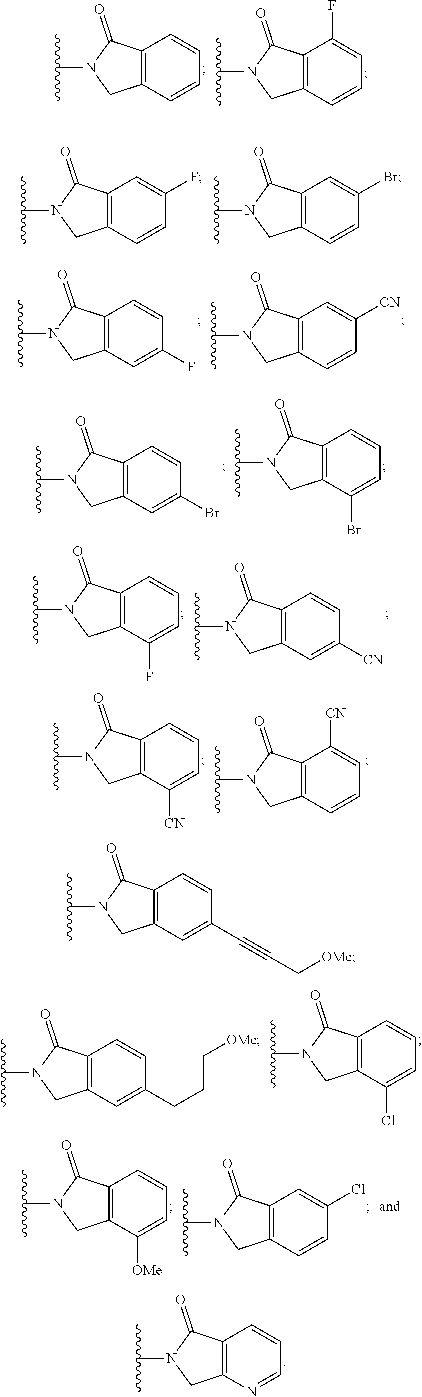

In any aspect or embodiment described herein, R₁₄ₐ, R_{14b} of Formula ULM-b, are each independently selected from the group of H, optionally substituted haloalkyl, optionally substituted alkyl, optionally substituted alkoxy, optionally substituted hydroxyl alkyl, optionally substituted alkylamine, optionally substituted amide, optionally substituted alkyl-amide, optionally substituted alkyl-cyano, optionally substituted alkyl-phosphate, optionally substituted heteraolkyl, optionally substituted alkyl-heterocycloalkyl, optionally substituted alkoxy-heterocycloalkyl, COR₂₆, alkyl-COR₂₆, CH₂OR₃₀, CH₂NHR₃₀, CH₂NCH₃R₃₀, CONR₂₇ₐR_{27b}, CH₂CONR₂₇ₐR_{27b}, CH₂NHCOR₂₆, or CH₂NCH₃COR₂₆; and the other of R₁₄ₐ and R_{14b} is H; or R₁₄ₐ, R_{14b}, together with the carbon atom to which they are attached, form an optionally substituted 3- to 6-membered cycloalkyl, heterocycloalkyl, spirocycloalkyl or spiroheterocyclyl, wherein the spiroheterocyclyl is not epoxide or aziridine, the said spirocycloalkyl or spiroheterocycloalkyl itself being optionally substituted with an alkyl, a haloalkyl, or —COR₃₃ where R₃₃ is an alkyl or a haloalkyl, wherein R₃₀ is selected from H, alkyl, alkynylalkyl, cycloalkyl, heterocycloalkyl, cycloalkylalkyl, heterocycloalkylalkyl, arylalkyl or heteroarylalkyl further optionally substituted; R₂₆ and R₂₇ are as described above.

In any aspect or embodiment described herein, R₁₅ of Formula ULM-b is selected from H, halogen, CN, C≡CH, OH, NO₂, NR₂₇ₐR_{27b}, OR₂₇ₐ, CONR₂₇ₐR_{27b}, NR₂₇ₐCOR_{27b}, SO₂NR₂₇ₐR_{27b}, NR₂₇ₐ SO₂R_{27b}, optionally substituted alkyl, optionally substituted haloalkyl (e.g. optionally substituted fluoroalkyl), optionally substituted haloalkoxy, optionally substituted aryl, optionally substituted heteroaryl, optionally substituted cycloalkyl, or optionally substituted heterocyclyl wherein optional substitution of the said aryl, heteroaryl, cycloalkyl and heterocycloalkyl includes CH₂OR₃₀, CH₂NHR₃₀, CH₂NCH₃R₃₀, CONR₂₇ₐR_{27b}, CH₂CONR₂₇ₐR_{27b}, CH₂NHCOR₂₆, CH₂NCH₃COR₂₆ or

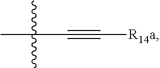

wherein R₂₆, R₂₇, R₃₀ and R₁₄ₐ are as described above.

In any aspect or embodiment described herein, R₁₄ₐ, R_{14b} of Formula ULM-b, are each independently selected from the group of H, optionally substituted haloalkyl, optionally substituted alkyl, CH₂OR₃₀, CH₂NHR₃₀, CH₂NCH₃R₃₀, CONR₂₇ₐR_{27b}, CH₂CONR₂₇ₐR_{27b}, CH₂NHCOR₂₆, or CH₂NCH₃COR₂₆; and the other of R₁₄ₐ and R_{14b} is H; or R₁₄ₐ, R_{14b}, together with the carbon atom to which they are attached, form an optionally substituted 3- to 6-membered spirocycloalkyl or spiroheterocyclyl, wherein the spiroheterocyclyl is not epoxide or aziridine, the said spirocycloalkyl or spiroheterocycloalkyl itself being optionally substituted with an alkyl, a haloalkyl, or —COR₃₃ where R₃₃ is an alkyl or a haloalkyl, wherein R₃₀ is selected from H, alkyl, alkynylalkyl, cycloalkyl, heterocycloalkyl, cycloalkylalkyl, heterocycloalkylalkyl, arylalkyl or heteroarylalkyl further optionally substituted; R₁₅ of Formula ULM-b is selected from H, halogen, CN, C° CH, OH, NO₂, NR₂₇ₐR_{27b}, OR₂₇ₐ, CONR₂₇ₐR_{27b}, NR₂₇ₐCOR_{27b}, SO₂NR₂₇ₐR_{27b}, NR₂₇ₐ SO₂R_{27b}, optionally substituted alkyl, optionally substituted haloalkyl, optionally substituted haloalkoxy, optionally substituted aryl, optionally substituted heteroaryl, optionally substituted cycloalkyl, or optionally substituted heterocyclyl wherein optional substitution of the said aryl, heteroaryl, cycloalkyl and heterocycloalkyl includes CH₂OR₃₀, CH₂NHR₃₀, CH₂NCH₃R₃₀, CONR₂₇ₐR_{27b}, CH₂CONR₂₇ₐR_{27b}, CH₂NHCOR₂₆, CH₂NCH₃COR₂₆ or

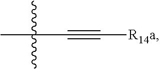

wherein R₂₆, R₂₇, R₃₀ and R₁₄a are as described above.

In certain embodiments, ULM has a chemical structure selected from:

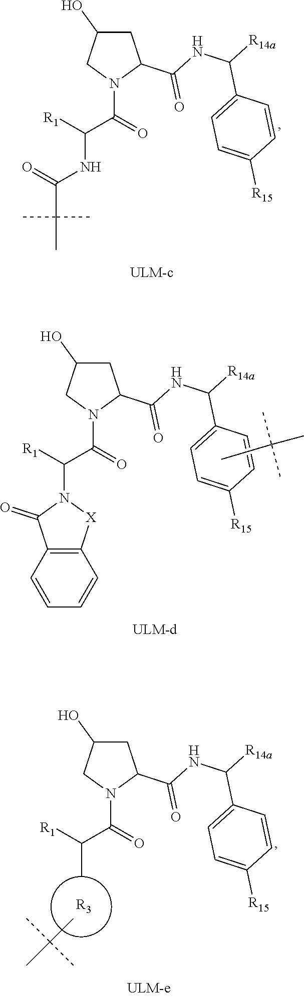

wherein:
R₁ of Formulas ULM-c, ULM-d, and ULM-e is H, ethyl, isopropyl, tert-butyl, sec-butyl, cyclopropyl, cyclobutyl, cyclopentyl, or cyclohexyl; optionally substituted alkyl, optionally substituted cycloalkyl, optionally substituted hydroxyalkyl, optionally substituted heteroaryl, or haloalkyl;
R₁₄ₐ of Formulas ULM-c, ULM-d, and ULM-e is H, haloalkyl, optionally substituted alkyl, methyl, fluoromethyl, hydroxymethyl, ethyl, isopropyl, or cyclopropyl;
R₁₅ of Formulas ULM-c, ULM-d, and ULM-e is selected from the group consisting of H, halogen, CN, C≡CH, OH, NO₂, optionally substituted heteroaryl, optionally substituted aryl; optionally substituted alkyl, optionally substituted haloalkyl, optionally substituted haloalkoxy, optionally substituted cycloalkyl, or optionally substituted heterocyclyl;
X of Formulas ULM-c, ULM-d, and ULM-e is C, CH₂, or C═O
R₃ of Formulas ULM-c, ULM-d, and ULM-e is absent or an optionally substituted 5 or 6 membered heteroaryl; and
the dashed line indicates the site of attachment of at least one PTM, another ULM or a chemical linker moiety coupling at least one PTM or both to ULM.

In certain embodiments, ULM comprises a group according to the chemical structure:

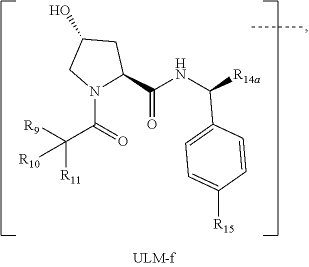

wherein:
R₁₄ₐ of Formula ULM-f is H, haloalkyl, optionally substituted alkyl, methyl, fluoromethyl, hydroxymethyl, ethyl, isopropyl, or cyclopropyl;
R₉ of Formula ULM-f is H;
R₁₀ of Formula ULM-f is H, ethyl, isopropyl, tert-butyl, sec-butyl, cyclopropyl, cyclobutyl, cyclopentyl, or cyclohexyl;
R₁₁ of Formula ULM-f is

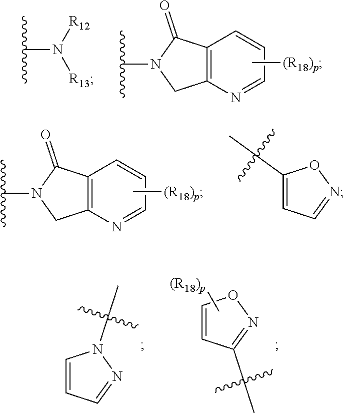

or optionally substituted heteroaryl;
p of Formula ULM-f is 0, 1, 2, 3, or 4;
each R₁₈ of Formula ULM-f is independently halo, optionally substituted alkoxy, cyano, optionally substituted alkyl, haloalkyl, haloalkoxy or a linker;
R₁₂ of Formula ULM-f is H, C═O;
R₁₃ of Formula ULM-f is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl,
R₁₅ of Formula ULM-f is selected from the group consisting of H, halogen, Cl, CN, C≡CH, OH, NO₂, optionally substituted haloalkyl, optionally substituted heteroaryl, optionally substituted aryl;

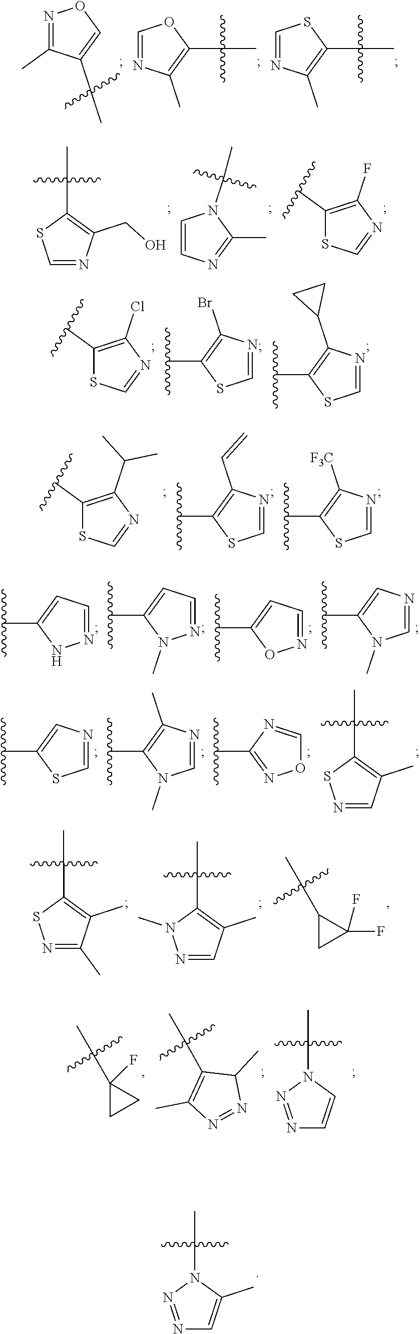

and
wherein the dashed line of Formula ULM-f indicates the site of attachment of at least one PTM, another ULM or a chemical linker moiety coupling at least one PTM or both to ULM.

In certain embodiments, the ULM is selected from:

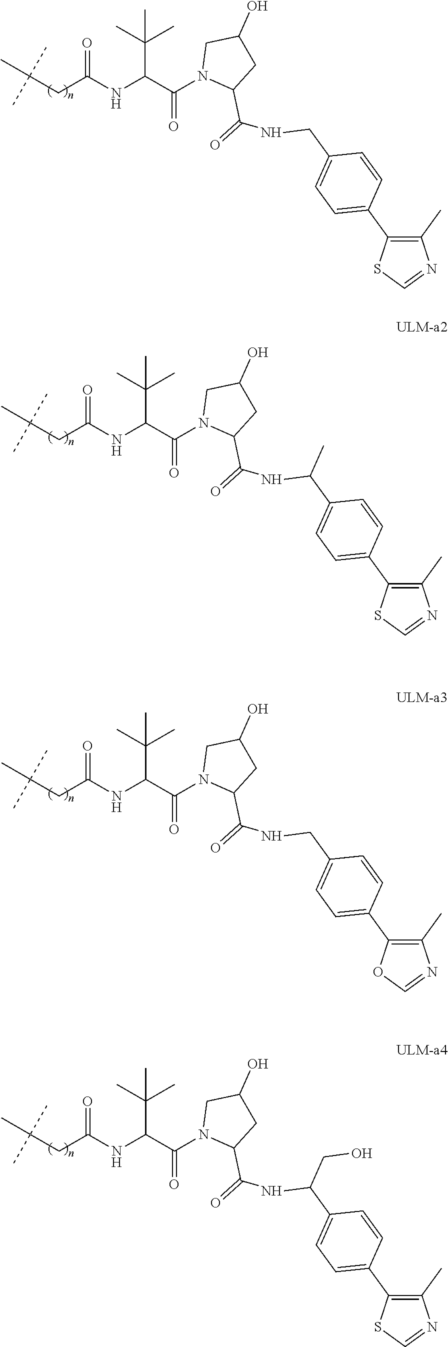

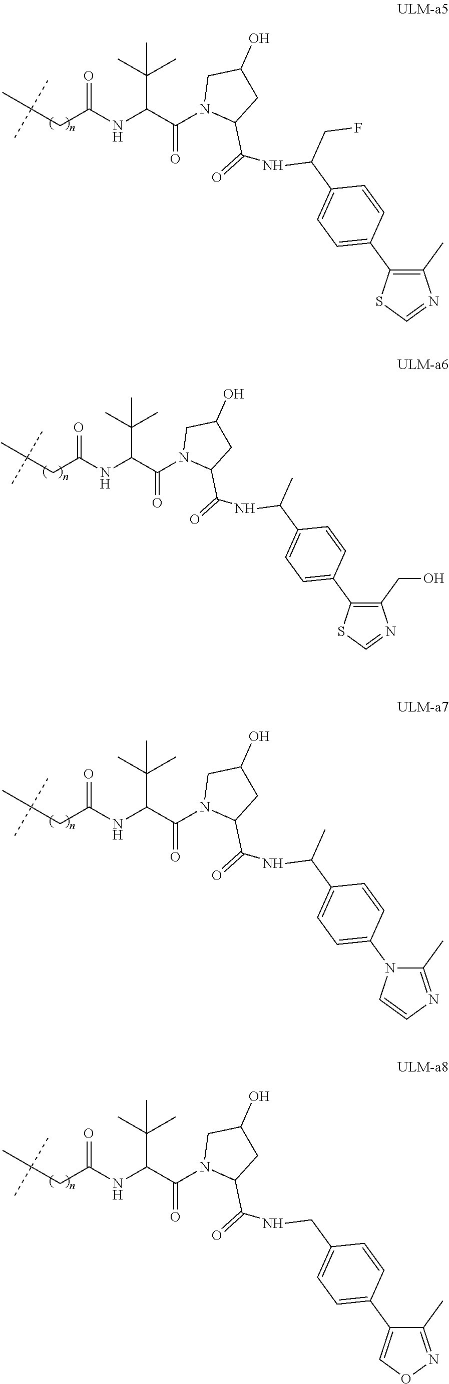

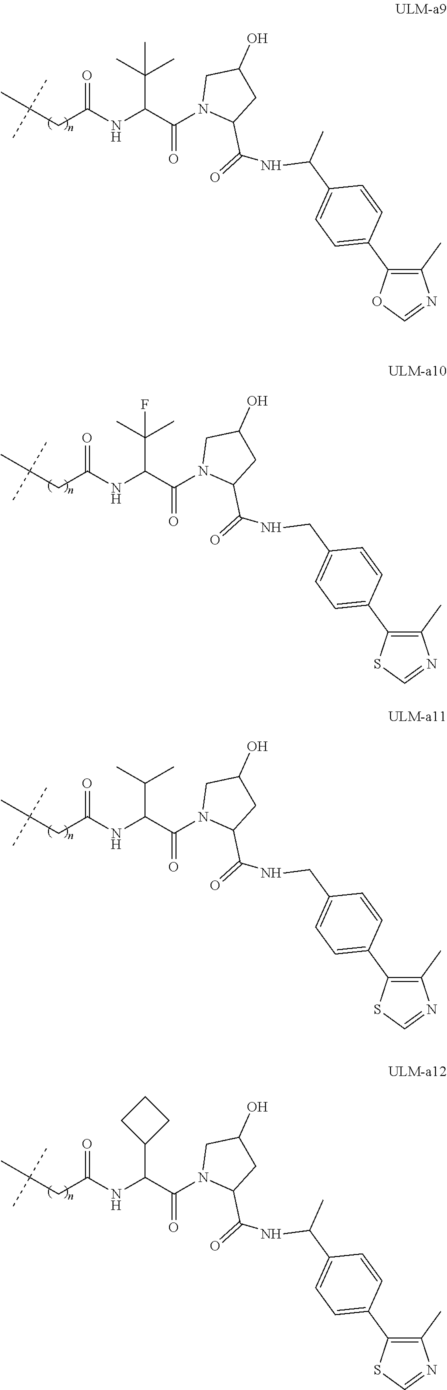

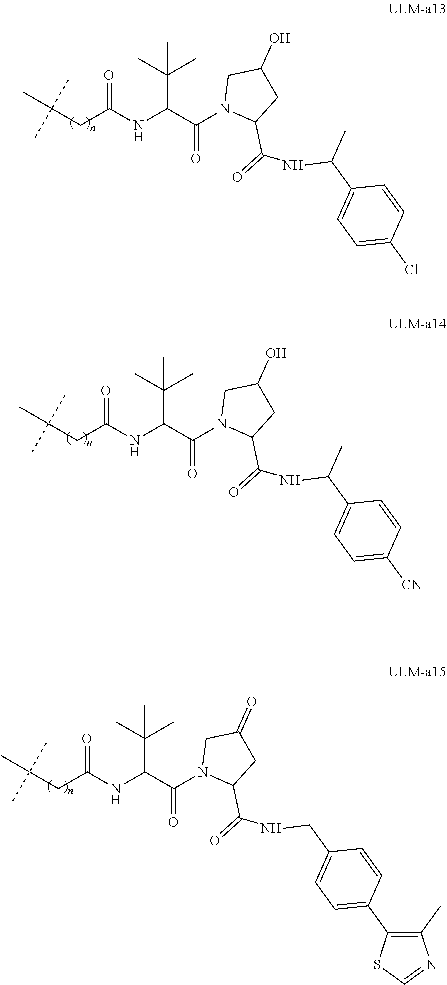

wherein n is 0 or 1.

In certain embodiments, the ULM is selected from:

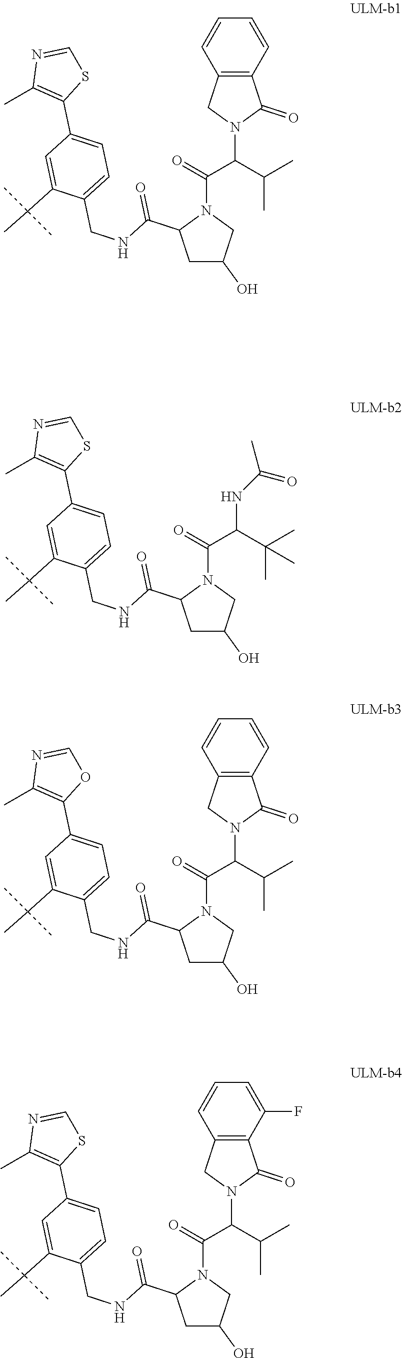

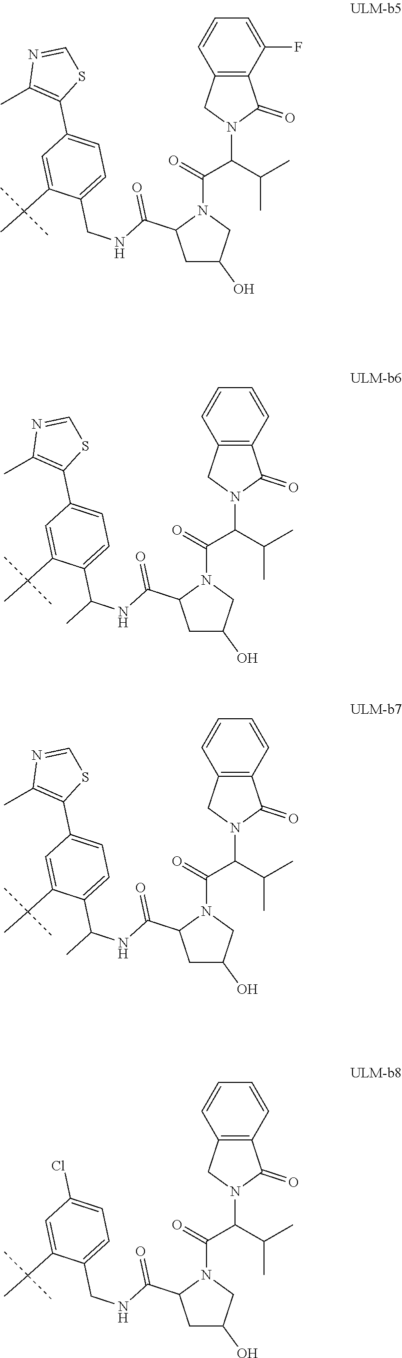

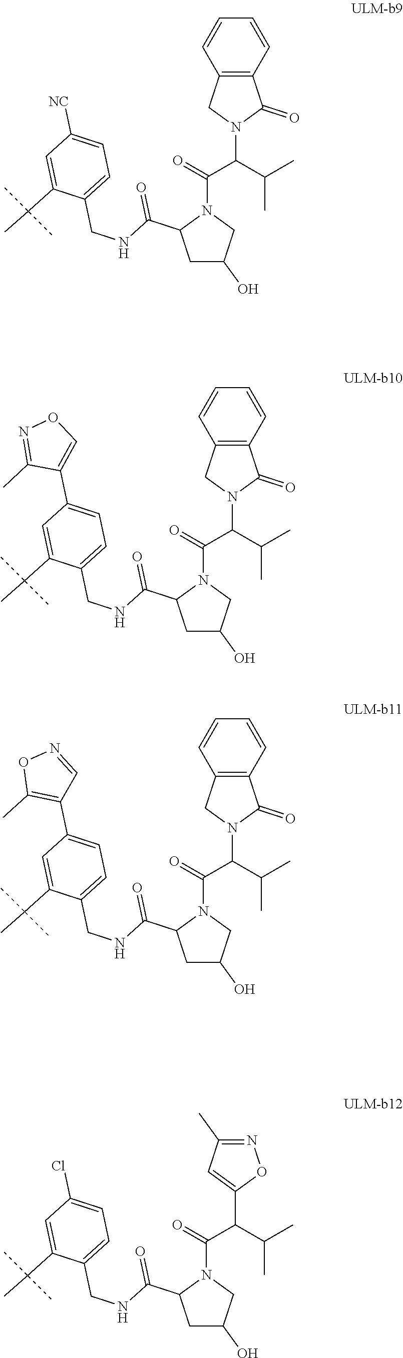

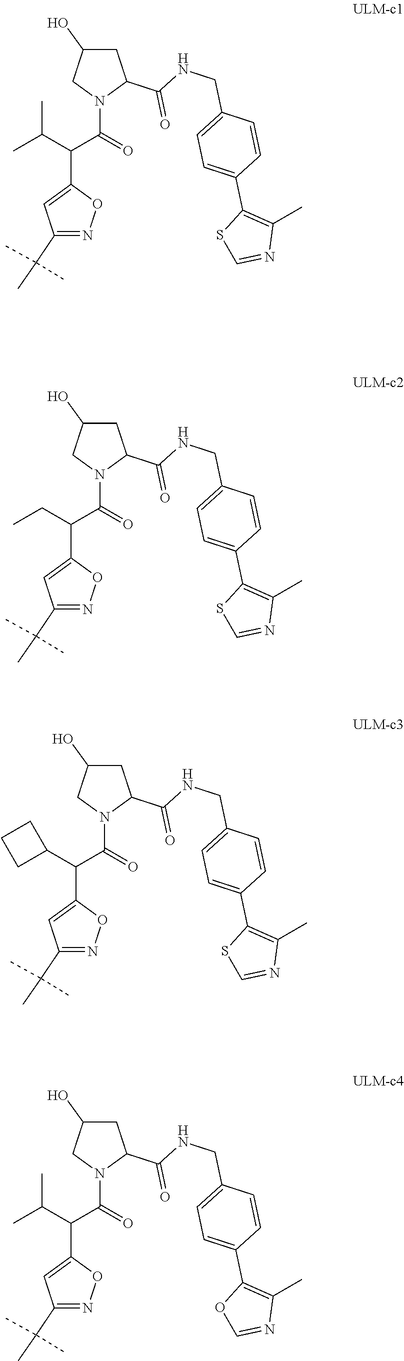

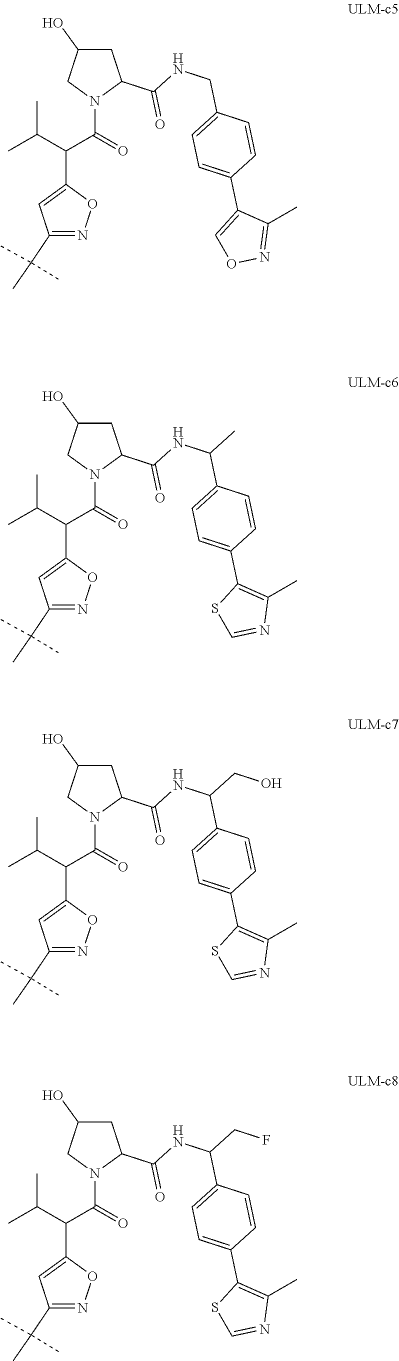

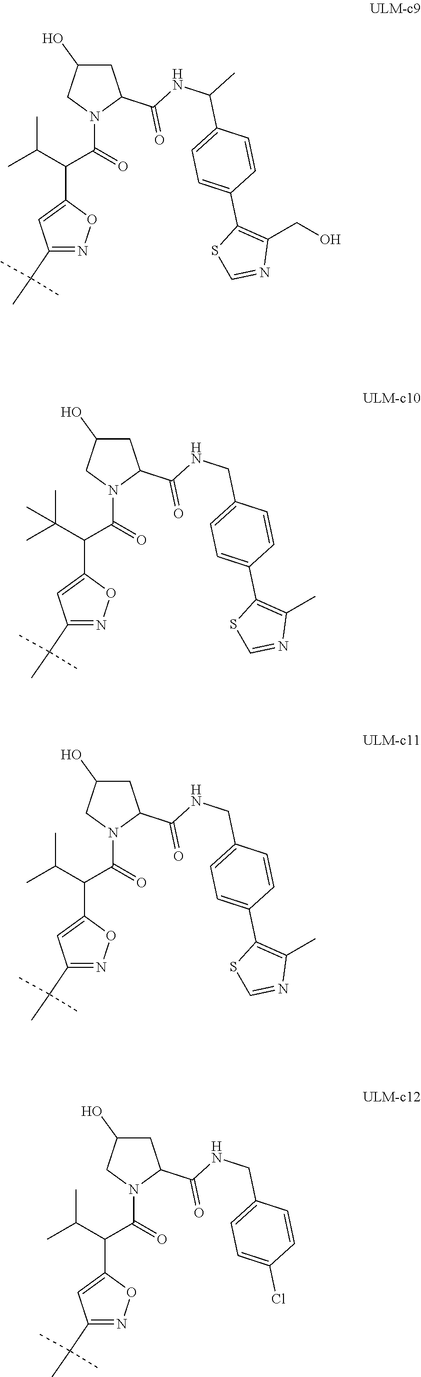

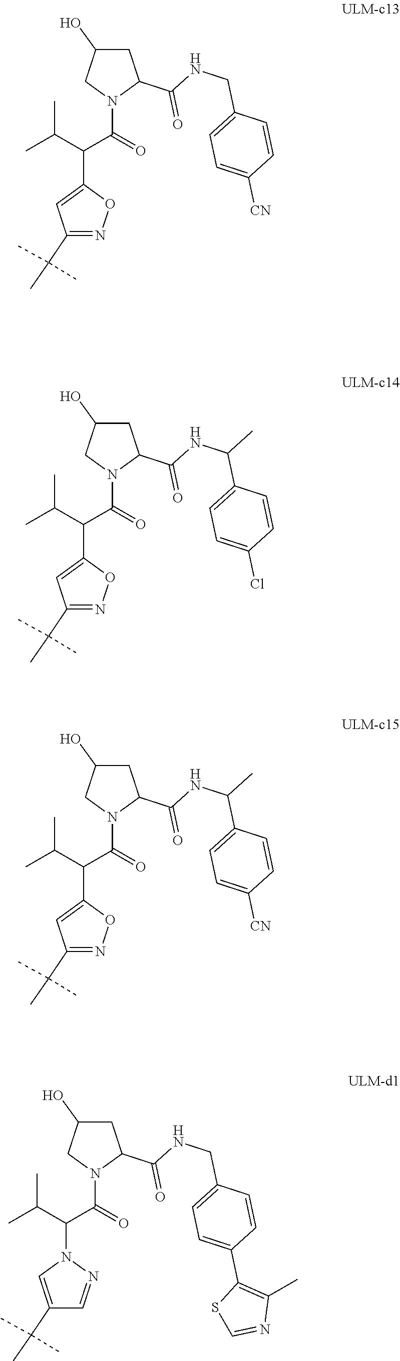

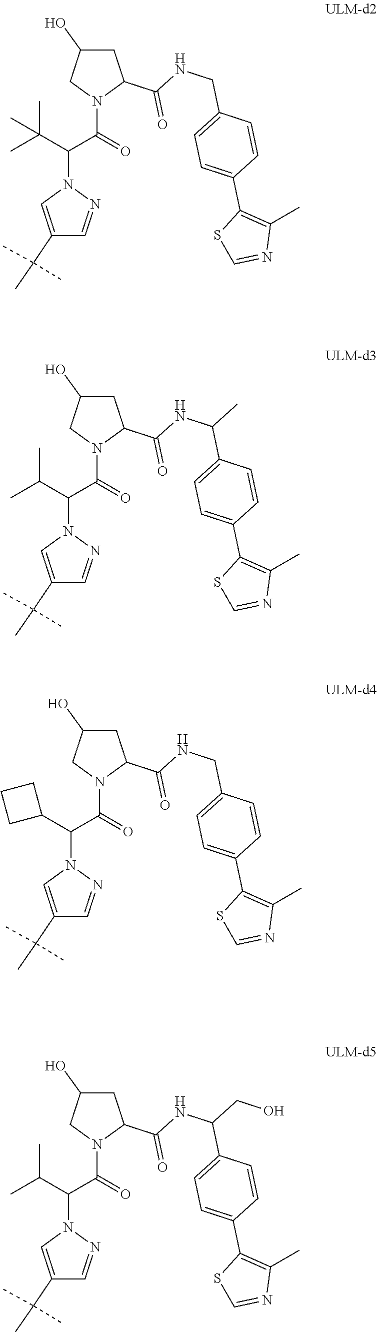

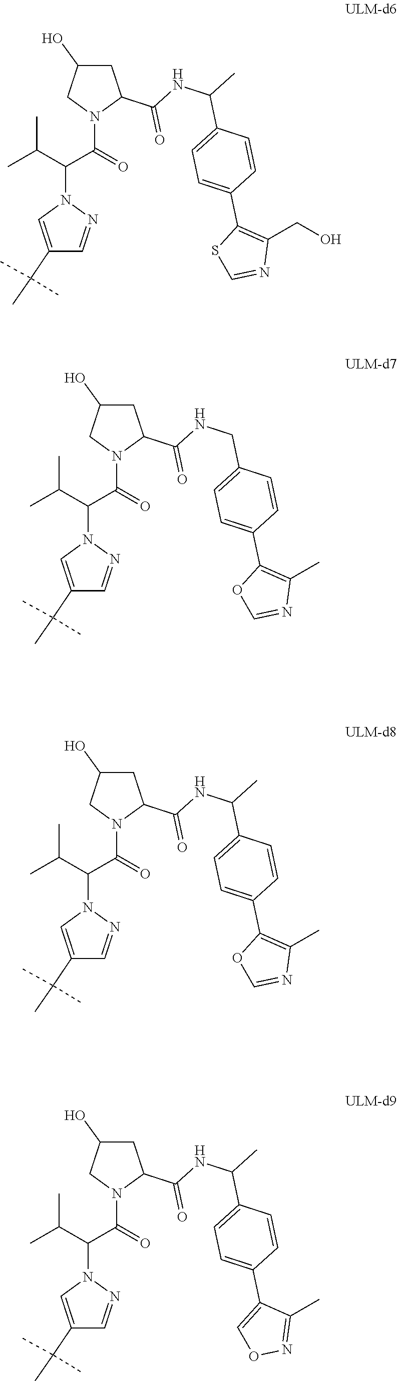

wherein, the phenyl ring in ULM-al through ULM-a15, ULM-b1 through ULM-b12, ULM-c1 through ULM-c15 and ULM-d1 through ULM-d9 is optionally substituted with fluorine, lower alkyl and alkoxy groups, and wherein the dashed line indicates the site of attachment of at least one PTM, another ULM or a chemical linker moiety coupling at least one PTM or both to ULM-a.

In certain embodiments, the hydroxyl group on the pyrrolidine ring of ULM-al through ULM-a15, ULM-b1 through ULM-b12, ULM-c1 through ULM-c15 and ULM-d1 through ULM-d9, respectively, comprises an ester-linked prodrug moiety.

In one embodiment, the phenyl ring in ULM-al through ULM-a15, ULM-b1 through ULM-b12, ULM-c1 through ULM-c15 and ULM-d1 through ULM-d9 can be functionalized as the ester to make it a part of the prodrug.

In any of the aspects or embodiments described herein, the ULM is a group according to the chemical structure:

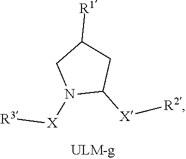

or a pharmaceutically acceptable salt thereof, wherein:
R^{1′} of ULM-g is an optionally substituted C₁₋₆ alkyl group, an optionally substituted —(CH₂)ₙOH, an optionally substituted —(CH₂)ₙSH, an optionally substituted (CH₂)ₙ—O—(C₁₋₆)alkyl group, an optionally substituted (CH₂)ₙ—WCOCW—(C₀₋₆)alkyl group containing an epoxide moiety WCOCW where each W is independently H or a C₁₋₃ alkyl group, an optionally substituted —(CH₂)ₙCOOH, an optionally substituted —(CH₂)ₙC(O)—(C₁₋₆ alkyl), an optionally substituted —(CH₂)ₙNHC(O)—R″, an optionally substituted —(CH₂)ₙC(O)—N(R″)₂, an optionally substituted —(CH₂)ₙOC(O)—N(R″)₂, —(CH₂O)ₙH, an optionally substituted —(CH₂)ₙOC(O)—(C₁₋₆ alkyl), an optionally substituted —(CH₂)C(O)—O—(C₁₋₆ alkyl), an optionally substituted —(CH₂O)ₙCOOH, an optionally substituted —(OCH₂)ₙO—(C₁₋₆ alkyl), an optionally substituted —(CH₂O)ₙC(O)—(C₁-C₆ alkyl), an optionally substituted —(OCH₂)ₙNHC(O)—R″, an optionally substituted —(CH₂O)ₙC(O)—N(R″)₂, —(CH₂CH₂O)ₙH, an optionally substituted —(CH₂CH₂O)ₙCOOH, an optionally substituted —(OCH₂CH₂)ₙO—(C₁₋₆ alkyl), an optionally substituted —(CH₂CH₂O)ₙC(O)—(C₁₋₆ alkyl), an optionally substituted —(OCH₂CH₂)ₙNHC(O)—R″, an optionally substituted —(CH₂CH₂O)ₙC(O)—N(R″)₂, an optionally substituted —SO₂R_{S}, an optionally substituted S(O)R_{S}, NO₂, CN or halogen (preferably F or Cl);
each R″ of ULM-g is independently H or a C₁₋₆ alkyl group which may be optionally substituted with one or two hydroxyl groups or up to three halogen groups (preferably fluorine);
R_{S} of ULM-g is a C₁₋₆ alkyl group, an optionally substituted aryl, heteroaryl or heterocyclyl group or a —(CH₂)ₘ N(R″)₂ group;
X and X′ of ULM-g are each independently C═O, C═S, —S(O), S(O)₂, (preferably X and X′ are both C═O);
R^{2′} of ULM-g is an optionally substituted —(CH₂)ₙ—(C═O)ᵤ(NR″)ᵥ(SO₂)_{w}alkyl group, an optionally substituted —(CH₂)ₙ—(C═O)ᵤ(NR″)ᵥ(SO₂)_{w}NR_{1N}R_{2N} group, an optionally substituted —(CH₂)ₙ—(C═O)ᵤ(NR″)ᵥ(SO₂)_{w}-Aryl, an optionally substituted —(CH₂)ₙ—(C═O)ᵤ(NR″)ᵥ(SO₂)_{w}-Heteroaryl, an optionally substituted —(CH₂)ₙ—(C═O)ᵥNR″ (SO₂)_{w}-heterocyclyl, an optionally substituted —NR″—(CH₂)ₙ—C(O)ᵤ(NR″)ᵥ(SO₂)_{w}-alkyl, an optionally substituted —NR″—(CH₂)ₙ—C(O)ᵤ(NR″)ᵥ(SO₂)_{w}—NR_{1N}R_{2N}, an optionally substituted —NR″—(CH₂)ₙ—C(O)ᵤ(NR″)ᵥ(SO₂)_{w}—NR″C(O)R_{1N}, an optionally substituted —NR″—(CH₂)ₙ—(C═O)ᵤ(NR″)ᵥ(SO₂)_{w}-aryl, an optionally substituted —NR″—(CH₂)ₙ—(C═O)ᵤ(NR″)ᵥ(SO₂)_{w}-heteroaryl or an optionally substituted —NR″—(CH₂)ₙ—(C═O)ᵥNR″ (SO₂)_{w}-Heterocyclyl, an optionally substituted —X^{R2′}-alkyl group; an optionally substituted —X^{R2′}-aryl group; an optionally substituted —X^{R2′}-heteroaryl group; an optionally substituted —X^{R2′}-heterocyclyl group;
R^{3′} of ULM-g is an optionally substituted alkyl, an optionally substituted —(CH₂)ₙ—(O)ᵤ(NR″)ᵥ(SO₂)_{w}-alkyl, an optionally substituted —(CH₂)ₙ—C(O)ᵤ(NR″)ᵥ(SO₂)_{w}—NR_{1N}R_{2N}, an optionally substituted —(CH₂)ₙ—C(O)ᵤ(NR″)ᵥ(SO₂)_{w}—NR″C(O)R_{1N}, an optionally substituted —(CH₂)ₙ—C(O)ᵤ(NR″)ᵥ(SO₂)_{w}—C(O)(R″)₂, an optionally substituted —(CH₂)ₙ—C(O)ᵤ(NR″)ᵥ(SO₂)_{w}-Aryl, an optionally substituted —(CH₂)ₙ—C(O)ᵤ(NR″)ᵥ(SO₂)_{w}-heteroaryl, an optionally substituted —(CH₂)ₙ—C(O)ᵤ(NR″)ᵥ(SO₂)_{w}-heterocyclyl, an optionally substituted —NR″—(CH₂)ₙ—C(O)ᵤ(NR″)ᵥ(SO₂)_{w}-alkyl, an optionally substituted —NR″—(CH₂)ₙ—C(O)ᵤ(NR″)ᵥ(SO₂)_{w}—NR_{1N}R_{2N}, an optionally substituted —NR″—(CH₂)ₙ—C(O)ᵤ(NR″)ᵥ(SO₂)_{w}—NR″C(O)R_{1N}, an optionally substituted —NR″—(CH₂)ₙ—C(O)ᵤ(NR″)ᵥ(SO₂)_{w}-aryl, an optionally substituted —NR″—(CH₂)ₙ—C(O)ᵤ(NR″)ᵥ(SO₂)_{w}-Heteroaryl, an optionally substituted —NR¹—(CH₂)ₙ—C(O)ᵤ(NR″)ᵥ(SO₂)_{w}-heterocyclyl, an optionally substituted —O—(CH₂)ₙ—(C═O)ᵤ(NR″)ᵥ(SO₂)_{w}-alkyl, an optionally substituted —O—(CH₂)ₙ—(C═O)ᵤ(NR″)ᵥ(SO₂)_{w}—NR_{1N}R_{2N}, an optionally substituted —O—(CH₂)ₙ—(C═O)ᵤ(NR″)ᵥ(SO₂)_{w}—NR″C(O)R_{1N}, an optionally substituted —O—(CH₂)ₙ—(C═O)ᵤ(NR″)ᵥ(SO₂)_{w}-aryl, an optionally substituted —O—(CH₂)ₙ—(C═O)ᵤ(NR″)ᵥ(SO₂)_{w}-Heteroaryl or an optionally substituted —O—(CH₂)ₙ—(C═O)ᵤ(NR″)ᵥ(SO₂)_{w}-heterocyclyl; —(CH₂)ₙ—(V)_{n′}—(CH₂)ₙ—(V)_{n′}-alkyl group, an optionally substituted —(CH₂)ₙ—(V)_{n′}—(CH₂)ₙ—(V)_{n′}-aryl group, an optionally substituted —(CH₂)ₙ—(V)₁—(CH₂)ₙ—(V)_{n′}-heteroaryl group, an optionally substituted —(CH₂)ₙ—(V)_{n′}—(CH₂)ₙ—(V)_{n′}-heterocyclyl′group, an optionally substituted —(CH₂)ₙ—N(R_{1′})(C═O)_{m′}—(V)_{n′}-alkyl group, an optionally substituted —(CH₂)ₙ—N(R_{1′})(C═O)_{m′}—(V)_{n′}-aryl group, an optionally substituted —(CH₂)ₙ—N(R_{1′})(C═O)_{m′}—(V)_{n′}-heteroaryl group, an optionally substituted —(CH₂)ₙ—N(R₁,)(C═O)_{m′}—(V)_{n′}-heterocyclyl group, an optionally substituted —X^{R3′}— alkyl group; an optionally substituted —X^{R3′}-aryl group; an optionally substituted —X^{R3′}-heteroaryl group; an optionally substituted —X^{R3′}— heterocyclyl group;
R_{1N} and R_{2N} of ULM-g are each independently H, C₁₋₆alkyl which is optionally substituted with one or two hydroxyl groups and up to three halogen groups or an optionally substituted —(CH₂)ₙ-aryl, —(CH₂)ₙ-heteroaryl or —(CH₂)ₙ-heterocyclyl group;
V of ULM-g is O, S, or NR₁;
each R_{1′} of ULM-g is independently H or a C₁₋₃alkyl group;
X^{R2′} and X^{R3′} of ULM-g are each independently an optionally substituted —(CH₂)ₙ—, —(CH₂)ₙ—CH(Xᵥ)═CH(Xᵥ)— (cis or trans), —CH₂)ₙ—CH≡CH—, —(CH₂CH₂O)ₙ—, or a C₃-C₆ cycloalkyl group, where Xᵥ is H, a halo, or a C₁-C₃ alkyl group that is optionally substituted;
each m of ULM-g is independently 0, 1, 2, 3, 4, 5, or 6;
each m′ of ULM-g is independently 0 or 1;
each n of ULM-g is independently 0, 1, 2, 3, 4, 5, or 6;
each n′ of ULM-g is independently 0 or 1;
each u of ULM-g is independently 0 or 1;
each v of ULM-g is independently 0 or 1;
each w of ULM-g is independently 0 or 1; and
any one or more of R^{1′}, R^{2′}, R^{3′}, X and X′ of ULM-g is optionally modified to be covalently bonded to the PTM group, or a pharmaceutically acceptable salt, stereoisomer, solvate or polymorph thereof.

In any of the aspects or embodiments described herein, the ULM is a group according to the chemical structure:

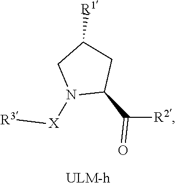

wherein:
each of R^{1′}, R^{2′} and R^{3′} of ULM-h are the same as above and X is C═O, C═S, —S(O) group or a S(O)₂ group, more preferably a C═O group, and
any one or more of R^{1′}, R^{2′} and R^{3′} of ULM-h are optionally modified to bind a linker group to which is further covalently bonded to the PTM group, or
a pharmaceutically acceptable salt, enantiomer, diastereomer, solvate or polymorph thereof.

In any of the aspects or embodiments described herein, the ULM has the chemical structure:

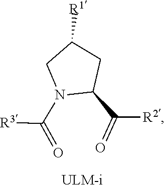

wherein:
any one or more of R^{1′}, R^{2′} and R^{3′} of ULM-I are optionally modified to bind a linker group to which is further covalently bonded to the PTM group, or
a pharmaceutically acceptable salt, enantiomer, diastereomer, solvate or polymorph thereof.

In further preferred aspects of the disclosure, R″ of ULM-g through ULM-i is preferably a hydroxyl group or a group which may be metabolized to a hydroxyl or carboxylic group, such that the compound represents a prodrug form of an active compound. Exemplary preferred R″ groups include, for example, —(CH₂)ₙOH, (CH₂)ₙ—O—(C₁-C₆)alkyl group, —(CH₂)ₙCOOH, —(CH₂O)ₙH, an optionally substituted —(CH₂)ₙOC(O)—(C₁-C₆ alkyl), or an optionally substituted —(CH₂)ₙC(O)—O—(C₁-C₆ alkyl), wherein n is 0 or 1. Where R^{1′} is or contains a carboxylic acid group, a hydroxyl group or an amine group, the hydroxyl group, carboxylic acid group or amine (each of which may be optionally substituted), may be further chemically modified to provide a covalent link to a linker group to which the PTM group is bonded.

In some embodiments, X and X′, where present, of ULM-g and ULM-h are preferably a C═O, C═S, —S(O) group or a S(O)₂ group, more preferably a C═O group.

In some embodiments, R^{2′} of ULM-g through ULM-i is preferably an optionally substituted —NH-T-aryl, an optionally substituted —N(CH₃)-T-aryl, an optionally substituted —NH-T-heteroaryl group, an optionally substituted —N(CH₃)-T-heteroaryl, an optionally substituted —NH-T-heterocyclyl, or an optionally substituted —N(CH₃)-T-heterocyclyl preferably H and T is an optionally substituted —(CH₂)ₙ— group, wherein each one of the methylene groups may be optionally substituted with one or two substituents, preferably selected from halogen, an amino acid sidechain as otherwise described herein or a C₁₋₃ alkyl group, preferably one or two methyl groups, which may be optionally substituted; and n is 0 to 6, often 0, 1, 2 or 3, preferably 0 or 1. Alternatively, T may also be a —(CH₂O)ₙ— group, a —(OCH₂)ₙ— group, a —(CH₂CH₂O)ₙ— group, a —(OCH₂CH₂)ₙ— group, all of which groups are optionally substituted.

Preferred aryl groups for R^{2′} of ULM-g through ULM-i include optionally substituted phenyl or naphthyl groups, preferably phenyl groups, wherein the phenyl or naphthyl group is connected to a PTM with a linker group and/or optionally substituted with a halogen (preferably F or Cl), an amine, monoalkyl- or dialkyl amine (preferably, dimethylamine), F, Cl, OH, COOH, C₁₋₆ alkyl, preferably CH₃, CF₃, OMe, OCF₃, NO₂, or CN group (each of which may be substituted in ortho-, meta- and/or para-positions of the phenyl ring, preferably para-), an optionally substituted phenyl group (the phenyl group itself is optionally connected to a PTM group with a linker group), and/or optionally substituted with at least one of F, Cl, OH, COOH, CH₃, CF₃, OMe, OCF₃, NO₂, or CN group (in the ortho-, meta- and/or para-positions of the phenyl ring, preferably in the para-position of the phenyl ring), a naphthyl group, which may be optionally substituted, an optionally substituted heteroaryl, preferably an optionally substituted isoxazole including a methylsubstituted isoxazole, an optionally substituted oxazole including a methylsubstituted oxazole, an optionally substituted thiazole including a methyl substituted thiazole, an optionally substituted isothiazole including a methyl substituted isothiazole, an optionally substituted pyrrole including a methylsubstituted pyrrole, an optionally substituted imidazole including a methylimidazole, an optionally substituted benzimidazole or methoxybenzylimidazole, an optionally substituted oximidazole or methyloximidazole, an optionally substituted diazole group, including a methyldiazole group, an optionally substituted triazole group, including a methylsubstituted triazole group, an optionally substituted pyridine group, including a halo-(preferably, F) or methylsubstitutedpyridine group or an oxapyridine group (where the pyridine group is linked to the phenyl group by an oxygen), an optionally substituted furan, an optionally substituted benzofuran, an optionally substituted dihydrobenzofuran, an optionally substituted indole, indolizine or azaindolizine (2, 3, or 4-azaindolizine), an optionally substituted quinoline, an optionally substituted group according to the chemical structure selected from:

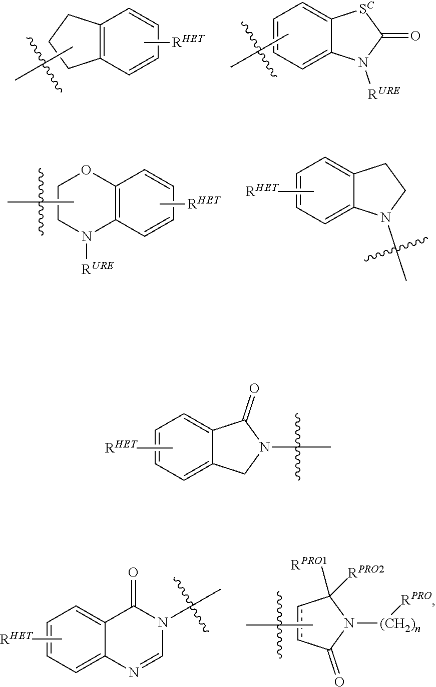

wherein:
S^{c} of ULM-g through ULM-i is CHR^{SS}, NR^{URE}, or O;
R^{HET} of ULM-g through ULM-i is H, CN, NO₂, halo (preferably Cl or F), optionally substituted C₁₋₆ alkyl (preferably substituted with one or two hydroxyl groups or up to three halogens (e.g., CF₃), optionally substituted O(C₁-C₆ alkyl) (preferably substituted with one or two hydroxyl groups or up to three halogens) or an optionally substituted acetylenic group —C≡C—Rₐ where Rₐ is H or a C₁₋₆ alkyl group (preferably C₁₋₃ alkyl);
R^{SS} of ULM-g through ULM-i is H, CN, NO₂, halo (preferably F or Cl), optionally substituted C₁₋₆ alkyl (preferably substituted with one or two hydroxyl groups or up to three halo groups), optionally substituted O—(C₁₋₆ alkyl) (preferably substituted with one or two hydroxyl groups or up to three halo groups) or an optionally substituted —C(O)(C₁₋₆ alkyl) (preferably substituted with one or two hydroxyl groups or up to three halo groups);
R^{URE} of ULM-g through ULM-i is H, a C₁-C₆ alkyl (preferably H or C₁-C₃ alkyl) or a —C(O)(C₁₋₆ alkyl) each of which groups is optionally substituted with one or two hydroxyl groups or up to three halogens (preferably fluorines), or an optionally substituted phenyl group, an optionally substituted heteroaryl, or an optionally substituted heterocyclyl, preferably for example piperidine, morpholine, pyrrolidine, tetrahydrofuran);
R^{PRO} of ULM-g through ULM-i is H, optionally substituted C₁₋₆ alkyl or an optionally substituted aryl (phenyl or napthyl), heteroaryl or heterocyclyl group selected from the group consisting of oxazole, isoxazole, thiazole, isothiazole, imidazole, diazole, oximidazole, pyrrole, pyrollidine, furan, dihydrofuran, tetrahydrofuran, thiene, dihydrothiene, tetrahydrothiene, pyridine, piperidine, piperazine, morpholine, quinoline, (each preferably substituted with a C₁₋₃ alkyl group, preferably methyl or a halogen (preferably flourine or chlorine), benzofuran, indole, indolizine, azaindolizine;
R^{PRO1} and R^{PRO2 of ULM-g through ULM-i} are each independently H, an optionally substituted C₁₋₃ alkyl group or together form a keto group; and
each n of ULM-g through ULM-i is independently 0, 1, 2, 3, 4, 5, or 6 (preferably 0 or 1), or an optionally substituted heterocyclyl, preferably tetrahydrofuran, tetrahydrothiene, piperidine, piperazine or morpholine (each of which groups when substituted, are preferably substituted with a methyl or halo (F, Br, C₁), each of which groups may be optionally attached to a PTM group via a linker group.

In certain embodiments,

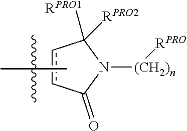

of ULM-g through ULM-i is a

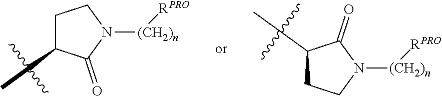

group,
where R^{PRO} and n of ULM-g through ULM-i are the same as above.

In some embodiments, the heteroaryl groups for R^{2′} of ULM-g through ULM-i include an optionally substituted quinoline (which may be attached to the pharmacophore or substituted on any carbon atom within the quinoline ring), an optionally substituted indole, an optionally substituted indolizine, an optionally substituted azaindolizine, an optionally substituted benzofuran, including an optionally substituted benzofuran, an optionally substituted isoxazole, an optionally substituted thiazole, an optionally substituted isothiazole, an optionally substituted thiophene, an optionally substituted pyridine (2-, 3-, or 4-pyridine), an optionally substituted imidazole, an optionally substituted pyrrole, an optionally substituted diazole, an optionally substituted triazole, a tetrazole, an optionally substituted oximidazole.

In some embodiments, the heteroaryl groups for R^{2′} of ULM-g through ULM-i is a group selected from:

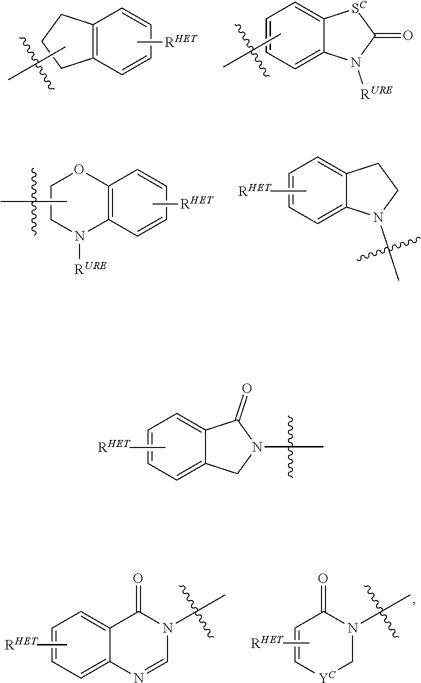

wherein:
S^{c} of ULM-g through ULM-i is CHR^{SS}, NR^{URE}, or O;
R^{HET} of ULM-g through ULM-i is H, CN, NO₂, halo (preferably Cl or F), optionally substituted C₁-C₆ alkyl (preferably substituted with one or two hydroxyl groups or up to three halogens (e.g. CF₃), optionally substituted O(C₁₋₆ alkyl) (preferably substituted with one or two hydroxyl groups or up to three halogens) or an optionally substituted acetylenic group —C≡C—Rₐ where Rₐ of ULM-g through ULM-i is H or a C₁₋₆ alkyl group (preferably C₁₋₃ alkyl);
R^{SS} of ULM-g through ULM-i is H, CN, NO₂, halo (preferably F or Cl), optionally substituted C₁₋₆ alkyl (preferably substituted with one or two hydroxyl groups or up to three halo groups), optionally substituted O—(C₁₋₆ alkyl) (preferably substituted with one or two hydroxyl groups or up to three halo groups) or an optionally substituted —C(O)(C₁₋₆ alkyl) (preferably substituted with one or two hydroxyl groups or up to three halogens);
R^{URE} of ULM-g through ULM-i is H, a C₁-C₆ alkyl (preferably H or C₁₋₃ alkyl) or a —C(O)(C₁₋₆ alkyl), each of which groups is optionally substituted with one or two hydroxyl groups or up to three halogens (preferably fluorine), or an optionally substituted heterocyclyl, for example piperidine, morpholine, pyrrolidine, tetrahydrofuran, tetrahydrothiophene, piperidine, piperazine, each of which is optionally substituted, and
Y^{C} of ULM-g through ULM-i is N or C—R^{YC}, where R^{YC} is H, OH, CN, NO₂, halo (preferably Cl or F), optionally substituted C₁₋₆ alkyl (preferably substituted with one or two hydroxyl groups or up to three halo groups (e.g. CF₃), optionally substituted O(C₁₋₆ alkyl) (preferably substituted with one or two hydroxyl groups or up to three halo groups) or an optionally substituted acetylenic group —C≡C—Rₐ where Rₐ is H or a C₁₋₆ alkyl group (preferably C₁₋₃ alkyl), each of which groups may be optionally connected to a PTM group via a linker group.

In some embodiments, the heterocyclylheterocyclyl groups for R^{2′} of ULM-g through ULM-i include tetrahydrofuran, tetrahydrothiene, tetrahydroquinoline, piperidine, piperazine, pyrrollidine, morpholine, oxane, or thiane, each of which groups may be optionally substituted.

In some embodiments, R^{2′} of ULM-g through ULM-i is a group selected from:

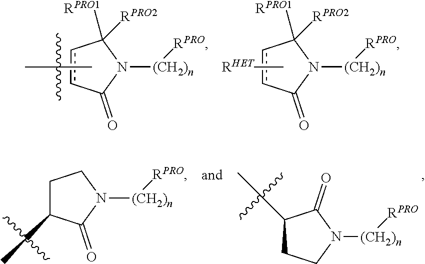

wherein:
R^{PRO} of ULM-g through ULM-i is H, optionally substituted C₁₋₆ alkyl or an optionally substituted aryl, heteroaryl or heterocyclyl group;
R^{PRO1} and R^{PRO2} of ULM-g through ULM-i are each independently H, an optionally substituted C₁-C₃ alkyl group or together form a keto group and
each n of ULM-g through ULM-i is independently 0, 1, 2, 3, 4, 5, or 6 (often 0 or 1), each of which groups can be optionally connected to a PTM group via a linker group.

In some embodiments, R^{2′} substituents of ULM-g through ULM-i also include specifically (and without limitation to the specific compound disclosed) the R^{2′} substituents which are found in the identified compounds disclosed herein (which includes the specific compounds which are disclosed in the present specification, and the figures which are attached hereto). Each of these R^{2′} substituents can be used in conjunction with any number of R^{3′} substituents which are also disclosed herein.

In some embodiments, R³, of ULM-g through ULM-i is an optionally substituted —NH-T-aryl, an optionally substituted —N(C₁-C₃ alkyl)-T-aryl, an optionally substituted —NH-T-heteroaryl group, an optionally substituted —N(C₁₋₃ alkyl)-T-heteroaryl, an optionally substituted —NH-T-heterocyclyl, or an optionally substituted —N(C₁-C₃ alkyl)-T-heterocyclyl, wherein T is an optionally substituted —(CH₂)ₙ— group, wherein each one of the methylene groups can be optionally substituted with one or two substituents, wherein the substituents can be selected from halogen, a C₁-C₃ alkyl group (e.g., methyl), or the sidechain of an amino acid as otherwise described herein, preferably methyl, each of which may be optionally substituted; and n is 0 to 6, often n is 0, 1, 2, or 3, preferably n is 0 or 1. Alternatively, T is a —(CH₂O)ₙ— group, a —(OCH₂)ₙ— group, a —(CH₂CH₂O)ₙ— group, or a —(OCH₂CH₂)ₙ— group, each of which groups can be optionally substituted.

In some embodiments, the aryl groups for R^{3′} of ULM-g through ULM-i include optionally substituted phenyl or naphthyl groups, preferably phenyl groups, wherein the phenyl or naphthyl group is optionally connected to a PTM group via a linker group and/or optionally substituted with a halogen (preferably F or Cl), an amine, monoalkyl- or dialkyl amine (preferably, dimethylamine), an amido group (preferably a —(CH₂)ₘ—NR₁C(O)R₂ group where m, R₁ and R₂ are the same as above), a halo (often F or Cl),OH, CH₃, CF₃, OMe, OCF₃, NO₂, CN, or a S(O)₂R_{S} group (R_{S} is a a C₁₋₆ alkyl group, an optionally substituted aryl, heteroaryl or heterocyclyl group or a —(CH₂)ₘ(R″)₂ group), each of which may be substituted at the ortho-, meta- and/or para-positions of the phenyl ring, preferably at the para-position of the phenyl ring), or an aryl (preferably phenyl), heteroaryl, or heterocyclyl. Preferably said substituent phenyl group is an optionally substituted phenyl group (i.e., the substituent phenyl group itself is preferably substituted with at least one of F, Cl, OH, SH, COOH, CH₃, CF₃, OMe, OCF₃, NO₂, CN or a linker group to which is attached a PTM group, wherein the substitution occurs at the ortho-, meta-, and/or para-positions of the phenyl ring, preferably at the para-position of the phenyl ring), a naphthyl group, which may be optionally substituted including as described above, an optionally substituted heteroaryl (preferably an optionally substituted isoxazole including a methylsubstituted isoxazole, an optionally substituted oxazole including a methylsubstituted oxazole, an optionally substituted thiazole including a methyl substituted thiazole, an optionally substituted pyrrole including a methylsubstituted pyrrole, an optionally substituted imidazole including a methylimidazole, a benzylimidazole or methoxybenzylimidazole, an oximidazole or methyloximidazole, an optionally substituted diazole group, including a methyldiazole group, an optionally substituted triazole group, including a methylsubstituted triazole group, a pyridine group, including a halo- (preferably, F) or methylsubstitutedpyridine group or an oxapyridine group (where the pyridine group is linked to the phenyl group by an oxygen) or an optionally substituted heterocyclyl (tetrahydrofuran, tetrahydrothiophene, pyrrolidine, piperidine, morpholine, piperazine, tetrahydroquinoline, oxane or thiane. Each of the aryl, heteroaryl or heterocyclyl groups may be optionally connected to a PTM group via a linker group.

In some embodiments, R³ of ULM-g through ULM-i is an optionally substituted quinoline (which may be attached to the pharmacophore or substituted on any carbon atom within the quinoline ring), an optionally substituted indole (including dihydroindole), an optionally substituted indolizine, an optionally substituted azaindolizine (2-, 3-, or 4-azaindolizine), an optionally substituted benzimidazole, benzodiazole, benzoxofuran, an optionally substituted imidazole, an optionally substituted isoxazole, an optionally substituted oxazole (preferably methyl substituted), an optionally substituted diazole, an optionally substituted triazole, a tetrazole, an optionally substituted benzofuran, an optionally substituted thiophene, an optionally substituted thiazole (preferably methyl and/or thiol substituted), an optionally substituted isothiazole, an optionally substituted triazole (preferably a 1,2,3-triazole substituted with a methyl group, a triisopropylsilyl group, an optionally substituted —(CH₂)ₘ—O—C₁-C₆ alkyl group or an optionally substituted —(CH₂)ₘ C(O)—O—C₁-C₆ alkyl group), or an optionally substituted pyridine (2-, 3-, or 4-pyridine).

In some embodiments, R³ of ULM-g through ULM-i is a group selected from:

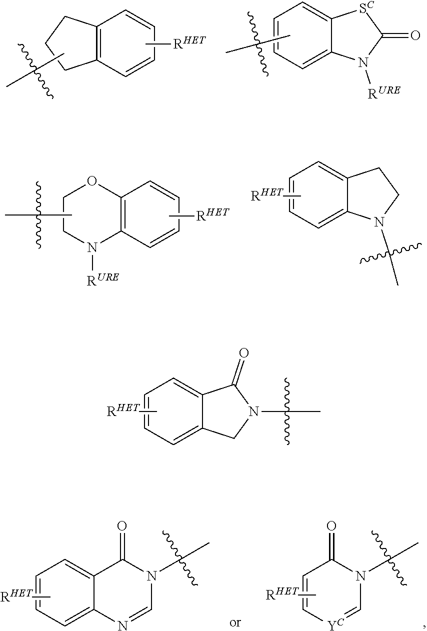

wherein:
S^{c} of ULM-g through ULM-i is CHR^{SS}, NR^{URE}, or O;
R^{HET} of ULM-g through ULM-i is H, CN, NO₂, halo (preferably Cl or F), optionally substituted C₁₋₆ alkyl (preferably substituted with one or two hydroxyl groups or up to three halo groups (e.g. CF₃), optionally substituted O(C₁₋₆ alkyl) (preferably substituted with one or two hydroxyl groups or up to three halo groups) or an optionally substituted acetylenic group —C≡C—Rₐ where Rₐ is H or a C₁₋₆ alkyl group (preferably C₁₋₃ alkyl);
R^{SS} of ULM-g through ULM-i is H, CN, NO₂, halo (preferably F or Cl), optionally substituted C₁₋₆ alkyl (preferably substituted with one or two hydroxyl groups or up to three halo groups), optionally substituted O—(C₁₋₆ alkyl) (preferably substituted with one or two hydroxyl groups or up to three halo groups) or an optionally substituted —C(O)(C₁₋₆ alkyl) (preferably substituted with one or two hydroxyl groups or up to three halogens;
R^{URE} of ULM-g through ULM-i is H, a C₁₋₆ alkyl (preferably H or C₁₋₃ alkyl) or a —C(O)(C₁₋₆ alkyl), each of which groups is optionally substituted with one or two hydroxyl groups or up to three halogen, preferably fluorine groups, or an optionally substituted heterocyclyl, for example piperidine, morpholine, pyrrolidine, tetrahydrofuran, tetrahydrothiophene, piperidine, piperazine, each of which is optionally substituted, and
Y^{C} of ULM-g through ULM-i is N or C—R^{YC}, where R^{YC} is H, OH, CN, NO₂, halo (preferably Cl or F), optionally substituted C₁₋₆ alkyl (preferably substituted with one or two hydroxyl groups or up to three halo groups (e.g. CF₃), optionally substituted O(C₁₋₆ alkyl) (preferably substituted with one or two hydroxyl groups or up to three halo groups) or an optionally substituted acetylenic group —C≡C—Rₐ where Rₐ is H or a C₁₋₆ alkyl group (preferably C₁₋₃ alkyl). Each of said heteroaryl groups may be optionally connected to a PTM group via a linker group.

In some embodiments, R^{3′} of ULM-g through ULM-i is tetrahydroquinoline, piperidine, piperazine, pyrrollidine, morpholine, tetrahydrofuran, tetrahydrothiophene, oxane and thiane, each of which groups may be optionally substituted.

In some embodiments, R^{3′} of ULM-g through ULM-i a group selected from:

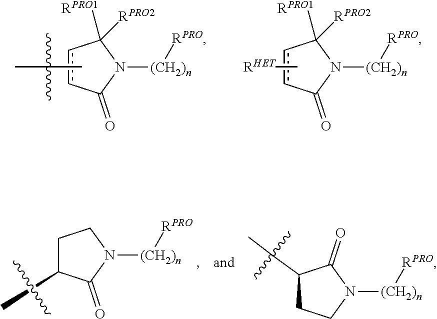

wherein:
R^{PRO} of ULM-g through ULM-i is H, optionally substituted C₁₋₆ alkyl or an optionally substituted aryl (phenyl or napthyl), heteroaryl or heterocyclyl group selected from the group consisting of oxazole, isoxazole, thiazole, isothiazole, imidazole, diazole, oximidazole, pyrrole, pyrollidine, furan, dihydrofuran, tetrahydrofuran, thiene, dihydrothiene, tetrahydrothiene, pyridine, piperidine, piperazine, morpholine, quinoline, (each preferably substituted with a C₁₋₃ alkyl group, preferably methyl or a halo group, preferably F or Cl), benzofuran, indole, indolizine, azaindolizine;
R^{PRO1} and R^{PRO2} of ULM-g through ULM-i are each independently H, an optionally substituted C₁-C₃ alkyl group or together form a keto group, and
each n of ULM-g through ULM-i is 0, 1, 2, 3, 4, 5, or 6 (preferably 0 or 1), wherein each of said heterocyclyl groups may be optionally connected to a PTM group via a linker group.

In some embodiments, R^{3′} substituents of ULM-g through ULM-i also include, but are not limited to, the R^{3′} substituents that are found in the identified compounds disclosed herein (which includes the specific compounds which are disclosed in the present specification, and the figures which are attached hereto). Each of these R^{3′} substituents can be used in conjunction with any number of R^{2′} substituents, which are also disclosed herein.

In certain alternative embodiments, R^{2′} of ULM-g through ULM-i is an optionally substituted —NR₁—X^{R2′}-alkyl group, —NR₁—X^{R2′}-aryl group; an optionally substituted —NR₁—X^{R2′}-HET, an optionally substituted —NR₁—X^{R2′}-aryl-HET or an optionally substituted —NR₁—X^{R2′}-HET-aryl, wherein:
R₁ of ULM-g through ULM-i is H or a C₁₋₃ alkyl group (preferably H);
X^{R2′} of ULM-g through ULM-i is an optionally substituted —CH₂)ₙ—, —CH₂)ₙ—CH(Xᵥ)═CH(Xₜ)-(cis or trans), —(CH₂)ₙ—CH—CH—, —(CH₂CH₂O)ₙ—, or a C₃-C₆ cycloalkyl group; and
Xᵥ of ULM-g through ULM-i is H, a halo or a C₁₋₃ alkyl group that is optionally substituted with one or two hydroxyl groups or up to three halogens;
Alkyl of ULM-g through ULM-i is an optionally substituted C₁₋₀ alkyl (preferably a C₁₋₆ alkyl) group (in certain preferred embodiments, the alkyl group is end-capped with a halogen, often a chlorine or a bromine);
Aryl of ULM-g through ULM-i is an optionally substituted phenyl or naphthyl group (preferably, a phenyl group); and
HET of ULM-g through ULM-i is an optionally substituted oxazole, isoxazole, thiazole, isothiazole, imidazole, diazole, oximidazole, pyrrole, pyrollidine, furan, dihydrofuran, tetrahydrofuran, thiene, dihydrothiene, tetrahydrothiene, pyridine, piperidine, piperazine, morpholine, benzofuran, indole, indolizine, azaindolizine, quinoline (when substituted, each preferably substituted with a C₁₋₃ alkyl group, preferably methyl or a halogen, preferably fluorine or chlorine), or is selected from:

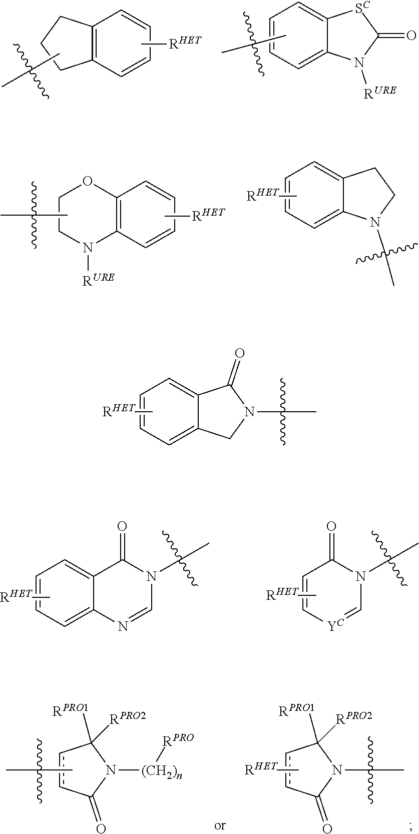

S^{c} of ULM-g through ULM-i is CHR^{SS}, NR^{URE}, or O;
R^{HET} of ULM-g through ULM-i is H, CN, NO₂, halogen (preferably chlorine or flourine), optionally substituted C₁-C₆ alkyl (preferably substituted with one or two hydroxyl groups or up to three halogens (e.g., CF₃), optionally substituted O(C₁₋₆ alkyl) (preferably substituted with one or two hydroxyl groups or up to three halogens) or an optionally substituted acetylenic group —C≡C—Rₐ where Rₐ is H or a C₁₋₆ alkyl group (preferably C₁₋₃ alkyl);
R^{SS} of ULM-g through ULM-i is H, CN, NO₂, halogen (preferably fluorine or chlorine), optionally substituted C₁₋₆ alkyl (preferably substituted with one or two hydroxyl groups or up to three halogens), optionally substituted O—(C₁₋₆ alkyl) (preferably substituted with one or two hydroxyl groups or up to three halogens) or an optionally substituted —C(O)(C₁₋₆ alkyl) (preferably substituted with one or two hydroxyl groups or up to three halogens);
R^{URE} of ULM-g through ULM-i is H, a C₁₋₆ alkyl (preferably H or C₁₋₃ alkyl) or a —C(O)(C₁₋₆ alkyl), each of which groups is optionally substituted with one or two hydroxyl groups or up to three halogens (preferably fluorine), or an optionally substituted heterocyclyl, for example piperidine, morpholine, pyrrolidine, tetrahydrofuran, tetrahydrothiophene, piperidine, piperazine, each of which is optionally substituted;
Y^{C} of ULM-g through ULM-i is N or C—R^{YC}, where R^{YC} is H, OH, CN, NO₂, halogen (preferably chlorine or flourine), optionally substituted C₁₋₆ alkyl (preferably substituted with one or two hydroxyl groups or up to three halo groups (e.g. CF₃), optionally substituted O(C₁₋₆ alkyl) (preferably substituted with one or two hydroxyl groups or up to three halogens) or an optionally substituted acetylenic group —C≡C—Rₐ where Rₐ is H or a C₁₋₆ alkyl group (preferably C₁₋₃ alkyl);
R^{PRO} of ULM-g through ULM-i is H, optionally substituted C₁₋₆ alkyl or an optionally substituted aryl (phenyl or napthyl), heteroaryl or heterocyclyl group selected from the group consisting of oxazole, isoxazole, thiazole, isothiazole, imidazole, diazole, oximidazole, pyrrole, pyrollidine, furan, dihydrofuran, tetrahydrofuran, thiene, dihydrothiene, tetrahydrothiene, pyridine, piperidine, piperazine, morpholine, quinoline, (each preferably substituted with a C₁₋₃ alkyl group, preferably methyl or a halogens (preferably fluorine or chlorine), benzofuran, indole, indolizine, azaindolizine;
R^{PRO1} and R^{PRO2} of ULM-g through ULM-i are each independently H, an optionally substituted C₁₋₃ alkyl group or together form a keto group, and
each n of ULM-g through ULM-i is independently 0, 1, 2, 3, 4, 5, or 6 (preferably 0 or 1), wherein each of the these groups is optionally connected to a PTM group via a linker group.

In some embodiments, R^{3′} of ULM-g through ULM-i is an optionally substituted —(CH₂)ₙ—(V)_{n′}—(CH₂)ₙ—(V)_{n′}—R^{S3′} group, an optionally substituted —(CH₂)ₙ—N(R_{1′})(C═O)_{m′}—(V)_{n′}—R^{S3′} group, an optionally substituted —X^{R3′}-alkyl group, an optionally substituted —X^{R3′}-aryl group; an optionally substituted —X^{R3′}-HET group, an optionally substituted —X^{R3′}-aryl-HET group or an optionally substituted —X^{R3′}-HET-aryl group, wherein:
R^{S3′} is an optionally substituted alkyl group (e.g., C₁₋₁₀ alkyl (preferably C₁₋₆ alkyl)), an optionally substituted aryl group, or a HET group;
R₁, is H or a C₁₋₃ alkyl group (preferably H);
V is O, S or NR_{1′};
X^{R3′} is —(CH₂)ₙ—, —(CH₂CH₂O)ₙ—, —(CH₂)ₙ—CH(Xᵥ)═CH(Xᵥ)— (cis or trans), —(CH₂)ₙ—CH≡CH—, or a C₃₋₆ cycloalkyl group, all of which can be optionally substituted;
Xᵥ is H, a halo, or a C₁₋₃ alkyl group that is optionally substituted with one or two hydroxyl groups or up to three halogens;
Alkyl is an optionally substituted C₁₋₁₀ alkyl (preferably a C₁₋₆ alkyl) group (in certain preferred embodiments, the alkyl group is end-capped with a halogen, often a chlorine or a bromine);
Aryl is an optionally substituted phenyl or napthyl group (preferably, a phenyl group); and
HET is an optionally substituted oxazole, isoxazole, thiazole, isothiazole, imidazole, diazole, oximidazole, pyrrole, pyrollidine, furan, dihydrofuran, tetrahydrofuran, thiene, dihydrothiene, tetrahydrothiene, pyridine, piperidine, piperazine, morpholine, benzofuran, indole, indolizine, azaindolizine, quinoline (when substituted, each preferably substituted with a C₁₋₃ alkyl group (preferably methyl) or a halogen (preferably fluorine or chlorine), or a group selected from:

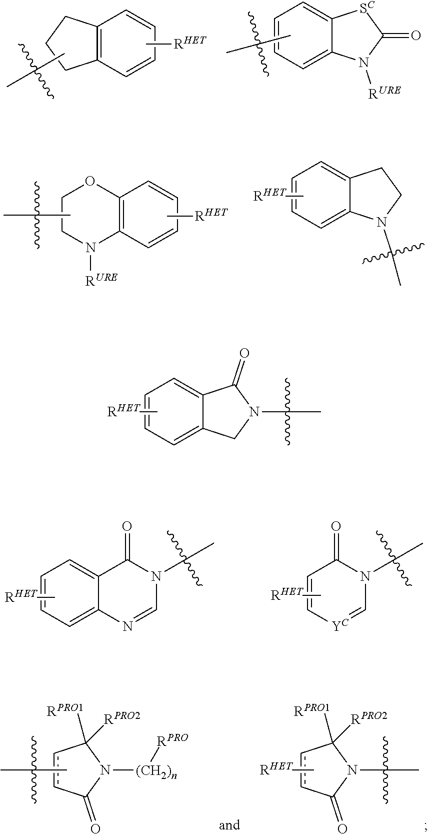

S^{c} of ULM-g through ULM-i is CHR^{SS}, NR^{URE}, or O;
R^{HET} of ULM-g through ULM-i is H, CN, NO₂, halo (preferably Cl or F), optionally substituted C₁₋₆ alkyl (preferably substituted with one or two hydroxyl groups or up to three halo groups (e.g. CF₃), optionally substituted O(C₁₋₆ alkyl) (preferably substituted with one or two hydroxyl groups or up to three halogens) or an optionally substituted acetylenic group —C≡C—Rₐ where Rₐ is H or a C₁₋₆ alkyl group (preferably C₁₋₃ alkyl);
R^{SS} of ULM-g through ULM-i is H, CN, NO₂, halogen (preferably fluorine or chlorine), optionally substituted C₁₋₆ alkyl (preferably substituted with one or two hydroxyl groups or up to three halo groups), optionally substituted O—(C₁₋₆ alkyl) (preferably substituted with one or two hydroxyl groups or up to three halogens) or an optionally substituted —C(O)(C₁₋₆ alkyl) (preferably substituted with one or two hydroxyl groups or up to three halogens);
R^{URE} of ULM-g through ULM-i is H, a C₁₋₆ alkyl (preferably H or C₁₋₃ alkyl) or a —C(O)(C₀₋₆ alkyl), each of which groups is optionally substituted with one or two hydroxyl groups or up to three halogens (preferably fluorine); or is an optionally substituted heterocyclyl (e.g., optionally substituted piperidinyl, optionally substituted morpholinyl, optionally substituted pyrrolidinyl, optionally substituted tetrahydrofuranyl, optionally substituted tetrahydrothiophenyl, optionally substituted piperidinyl, optionally substituted piperazinyl);
Y^{C} of ULM-g through ULM-i is N or C—R^{YC}, where R^{YC} is H, OH, CN, NO₂, halo (preferably chlorine or flourine), optionally substituted C₁₋₆ alkyl (preferably substituted with one or two hydroxyl groups or up to three halogens (e.g., CF₃), optionally substituted O(C₁₋₆ alkyl) (preferably substituted with one or two hydroxyl groups or up to three halogens), or an optionally substituted acetylenic group —C≡C—Rₐ where Rₐ is H or a C₁₋₆ alkyl group (preferably C₁₋₃ alkyl);
R^{PRO} of ULM-g through ULM-i is H, optionally substituted C₁₋₆ alkyl, an optionally substituted aryl (phenyl or napthyl), optionally substituted heteroaryl, or optionally substituted heterocyclyl, wherein optionally substituted heteroaryl or optionally substituted heterocyclyl is selected from optionally substituted oxazolyl, isoxazolyl, thiazolyl, isothiazolyl, imidazolyl, diazolyl, oximidazolyl, pyrrolyl, pyrollidinyl, furanyl, dihydrofuranyl, tetrahydrofuranyl, thienyl, dihydrothienyl, tetrahydrothienyl, pyridinyl, piperidinyl, piperazinyl, morpholinyl, quinolinyl, benzofuranyl, indolyl, indolizinyl, or azaindolizinyl, wherein the optional substitutents are selected from a C₁₋₃ alkyl group (e.g., methyl), halogen (e.g., F or Cl);
R^{PRO1} and R^{PRO2} of ULM-g through ULM-i are each independently H, optionally substituted C₁₋₃ alkyl group, or taken together form a keto group;
each n of ULM-g through ULM-i is independently 0, 1, 2, 3, 4, 5, or 6 (preferably 0 or 1);
each m′ of ULM-g through ULM-i is 0 or 1; and
each n′ of ULM-g through ULM-i is 0 or 1;
wherein the alkyl, aryl, or HET groups optionally connect to a PTM group via a linker.

In alternative embodiments, R^{3′} of ULM-g through ULM-i is —(CH₂)ₙ-aryl, —(CH₂CH₂O)ₙ-aryl, —(CH₂)ₙ-HET, or —(CH₂CH₂O)ₙ-HET, wherein:
Aryl of ULM-g through ULM-i is phenyl that is optionally substituted with one or two substitutents preferably selected from —(CH₂)ₙOH, C₁-C₆ alkyl (which can be further substituted with CN, up to three halogens, OH), —(CH₂)ₙO(C₁-C₆)alkyl, amine, mono- or di-(C₁₋₆ alkyl) amine (wherein the alkyl group on the amine is optionally substituted with 1 or 2 hydroxyl groups or up to three halogens (preferably fluorine and chlorine);
Aryl of ULM-g through ULM-i is —(CH₂)ₙOH, —(CH₂)ₙ—O—(C₁₋₆)alkyl, —(CH₂)ₙ—O—(CH₂)ₙ—(C₁₋₆)alkyl, —(CH₂)ₙ—C(O)(C₀₋₆) alkyl, —(CH₂)ₙ—C(O)O(C₀-C₆)alkyl, —(CH₂)ₙ—OC(O)(C₀₋₆)alkyl, amine, mono- or di-(C₁₋₆ alkyl) amine (wherein the alkyl group on the amine is optionally substituted with 1 or 2 hydroxyl groups or up to three halogens (preferably fluorine and chlorine)), CN, NO₂, an optionally substituted —(CH₂)ₙ—(V)_{m′}—CH₂)ₙ—(V)_{m′}—(C₁₋₆)alkyl, a —(V)_{m′}—(CH₂CH₂O)ₙ—R^{PEG}, where V is O, S or NR_{1′}, R_{1′}, is H or a C₁-C₃ alkyl group (preferably H) and R^{PEG} is H or a C₁₋₆ alkyl group which is optionally substituted (including being optionally substituted with a carboxyl group); or
Aryl of ULM-g through ULM-i is optionally substituted with a heterocyclyl, including a heteroaryl, selected from the group consisting of oxazole, isoxazole, thiazole, isothiazole, imidazole, diazole, oximidazole, pyrrole, pyrollidine, furan, dihydrofuran, tetrahydrofuran, thiene, dihydrothiene, tetrahydrothiene, pyridine, piperidine, piperazine, morpholine, quinoline, benzofuran, indole, indolizine, azaindolizine, (when substituted each preferably substituted with a C₁₋₃ alkyl group (preferably methyl) or a halogen (preferably fluorine or chlorine), or a group selected from:

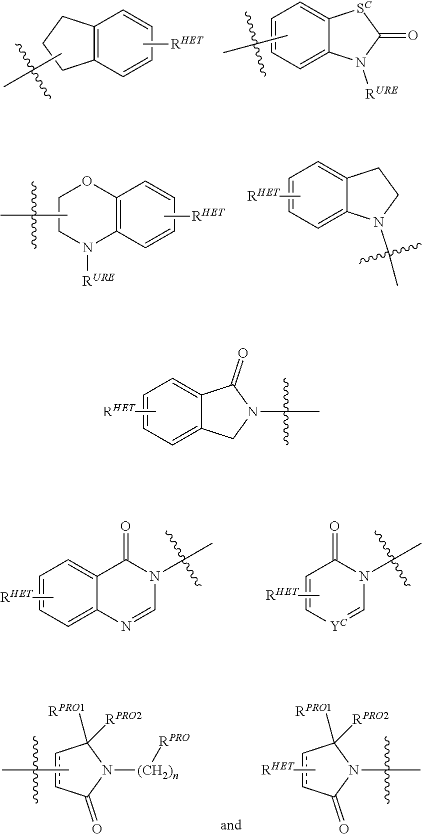

S^{c} of ULM-g through ULM-i is CHR^{SS}, NR^{URE}, or O;
R^{HET} of ULM-g through ULM-i is H, CN, NO₂, halo (preferably Cl or F), optionally substituted C₁₋₆ alkyl (preferably substituted with one or two hydroxyl groups or up to three halo groups (e.g. CF₃), optionally substituted O(C₁₋₆ alkyl) (preferably substituted with one or two hydroxyl groups or up to three halogens) or an optionally substituted acetylenic group —C≡C—Rₐ where Rₐ is H or a C₁₋₆ alkyl group (preferably C₁₋₃ alkyl);
R^{SS} of ULM-g through ULM-i is H, CN, NO₂, halogen (preferably fluorine or chlorine), optionally substituted C₁₋₆ alkyl (preferably substituted with one or two hydroxyl groups or up to three halo groups), optionally substituted O—(C₁₋₆ alkyl) (preferably substituted with one or two hydroxyl groups or up to three halogens) or an optionally substituted —C(O)(C₁₋₆ alkyl) (preferably substituted with one or two hydroxyl groups or up to three halogens);
R^{URE} of ULM-g through ULM-i is H, a C₁₋₆ alkyl (preferably H or C₁₋₃ alkyl) or a —C(O)(C₀₋₆ alkyl), each of which groups is optionally substituted with one or two hydroxyl groups or up to three halogen, preferably fluorine groups, or an optionally substituted heterocyclyl, for example piperidine, morpholine, pyrrolidine, tetrahydrofuran, tetrahydrothiophene, piperidine, piperazine, each of which is optionally substituted;
Y^{C} of ULM-g through ULM-i is N or C—R^{YC}, where R^{YC} is H, OH, CN, NO₂, halogen (preferably chlorine or fluorine), optionally substituted C₁₋₆ alkyl (preferably substituted with one or two hydroxyl groups or up to three halogens (e.g. CF₃), optionally substituted O(C₁₋₆ alkyl) (preferably substituted with one or two hydroxyl groups or up to three halogens) or an optionally substituted acetylenic group —C≡C—Rₐ where Rₐ is H or a C₁₋₆ alkyl group (preferably C₁₋₃ alkyl);
R^{PRO} of ULM-g through ULM-i is H, optionally substituted C₁₋₆ alkyl or an optionally substituted aryl (phenyl or napthyl), heteroaryl or heterocyclyl group selected from the group consisting of oxazole, isoxazole, thiazole, isothiazole, imidazole, diazole, oximidazole, pyrrole, pyrollidine, furan, dihydrofuran, tetrahydrofuran, thiene, dihydrothiene, tetrahydrothiene, pyridine, piperidine, piperazine, morpholine, quinoline, (each preferably substituted with a C₁₋₃ alkyl group, preferably methyl or a halogen, preferably fluorine or chlorine), benzofuran, indole, indolizine, azaindolizine;
R^{PRO1} and R^{PRO2} of ULM-g through ULM-i are each independently H, an optionally substituted C₁₋₃ alkyl group or together form a keto group; HET of ULM-g through ULM-i is preferably oxazole, isoxazole, thiazole, isothiazole, imidazole, diazole, oximidazole, pyrrole, pyrollidine, furan, dihydrofuran, tetrahydrofuran, thiene, dihydrothiene, tetrahydrothiene, pyridine, piperidine, piperazine, morpholine, quinoline, (each preferably substituted with a C₁₋₃ alkyl group (preferably methyl) or a halogen (preferably fluorine or chlorine), benzofuran, indole, indolizine, azaindolizine, or a group according to the chemical structure:

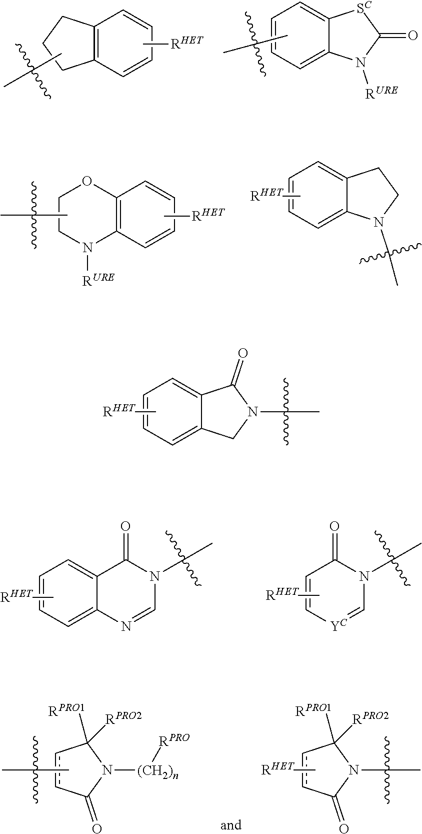

S^{c} of ULM-g through ULM-i is CHR^{SS}, NR^{URE}, or O;
R^{HET} of ULM-g through ULM-i is H, CN, NO₂, halo (preferably Cl or F), optionally substituted C₁₋₆ alkyl (preferably substituted with one or two hydroxyl groups or up to three halogens (e.g. CF₃)), optionally substituted O(C₁₋₆ alkyl) (preferably substituted with one or two hydroxyl groups or up to three halogens) or an optionally substituted acetylenic group —C≡C—Rₐ where Rₐ is H or a C₁₋₆ alkyl group (preferably C₁₋₃ alkyl);
R^{SS} of ULM-g through ULM-i is H, CN, NO₂, halo (preferably F or Cl), optionally substituted C₁-C₆ alkyl (preferably substituted with one or two hydroxyl groups or up to three halo groups), optionally substituted O—(C₁₋₆ alkyl) (preferably substituted with one or two hydroxyl groups or up to three halo groups) or an optionally substituted —C(O)(C₁₋₆ alkyl) (preferably substituted with one or two hydroxyl groups or up to three halo groups);
R^{URE} of ULM-g through ULM-i is H, a C₁₋₆ alkyl (preferably H or C₁₋₃ alkyl) or a —C(O)(C₀₋₆ alkyl), each of which groups is optionally substituted with one or two hydroxyl groups or up to three halogens (preferably fluorine), or an optionally substituted heterocyclyl, for example piperidine, morpholine, pyrrolidine, tetrahydrofuran, tetrahydrothiophene, piperidine, piperazine, each of which is optionally substituted;
Y^{C} of ULM-g through ULM-i is N or C—R^{YC}, where R^{YC} is H, OH, CN, NO₂, halogen (preferably chlorine or fluorine), optionally substituted C₁₋₆ alkyl (preferably substituted with one or two hydroxyl groups or up to three halogens (e.g., CF₃), optionally substituted O(C₁₋₆ alkyl) (preferably substituted with one or two hydroxyl groups or up to three halogens) or an optionally substituted acetylenic group —C≡C—Rₐ where Rₐ is H or a C₁₋₆ alkyl group (preferably C₁₋₃ alkyl);
R^{PRO} of ULM-g through ULM-i is H, optionally substituted C₁₋₆ alkyl, optionally substituted aryl, optionally substituted heteroaryl, or optionally substituted heterocyclyl;
R^{PRO1} and R^{PRO2} of ULM-g through ULM-i are each independently H, an optionally substituted C₁₋₃ alkyl group or together form a keto group;
each m′ of ULM-g through ULM-i is independently 0 or 1; and
each n of ULM-g through ULM-i is independently 0, 1, 2, 3, 4, 5, or 6 (preferably 0 or 1),
wherein each of said compounds, preferably on said Aryl or HET groups, is optionally connected to a PTM group via a linker group.

In still additional embodiments, preferred compounds include those according to the chemical structure:

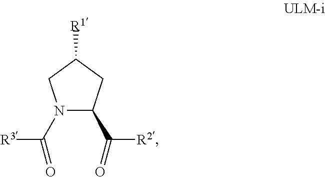

wherein:
R^{1′} of ULM-i is OH or a group which is metabolized in a patient or subject to OH;
R^{2′} of ULM-i is a —NH—CH₂-Aryl-HET (preferably, a phenyl linked directly to a methyl substituted thiazole);
R^{3′} of ULM-i is a —CHR^{CR3′}—NH—C(O)—R^{3P1} group or a —CHR^{CR3′}—R^{3P2} group; R^{CR3′} of ULM-i is a C₁₋₄ alkyl group, preferably methyl, isopropyl or tert-butyl;
R^{3P1} of ULM-i is C₁₋₃ alkyl (preferably methyl), an optionally substituted oxetane group (preferably methyl substituted), a —(CH₂)·OCH₃ group where n is 1 or 2 (preferably 2), or a

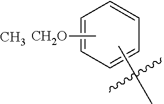

group (the ethyl ether group is preferably meta-substituted on the phenyl moiety), a morpholino group (linked to the carbonyl at the 2- or 3-position;

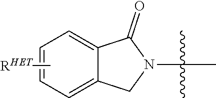

R^{3P2} of ULM-i is a group;
Aryl of ULM-i is phenyl;
HET of ULM-i is an optionally substituted thiazole or isothiazole; and
R^{HET} of ULM-i is H or a halo group (preferably H);
or a pharmaceutically acceptable salt, stereoisomer, solvate or polymorph thereof, wherein each of said compounds is optionally connected to a PTM group via a linker group.

In certain aspects, bifunctional compounds comprising a ubiquitin E3 ligase binding moiety (ULM), wherein ULM is a group according to the chemical structure:

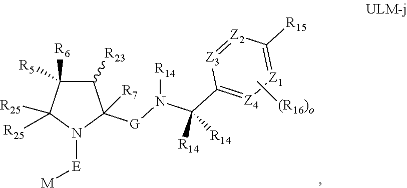

wherein:
each R₅ and R₆ of ULM-j is independently OH, SH, or optionally substituted alkyl or R₅, R₆, and the carbon atom to which they are attached form a carbonyl;
R₇ of ULM-j is H or optionally substituted alkyl;
E of ULM-j is a bond, C═O, or C═S;
G of ULM-j is a bond, optionally substituted alkyl, —COOH or C=J;
J of ULM-j is 0 or N—R⁸;
R⁸ of ULM-j is H, CN, optionally substituted alkyl or optionally substituted alkoxy;
M of ULM-j is optionally substituted aryl, optionally substituted heteroaryl, optionally substituted heterocyclyl or

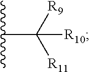

each R₉ and R₁₀ of ULM-j is independently H; optionally substituted alkyl, optionally substituted cycloalkyl, optionally substituted hydroxyalkyl, optionally substituted thioalkyl, a disulphide linked ULM, optionally substituted heteroaryl, or haloalkyl; or R₉, R₁₀, and the carbon atom to which they are attached form an optionally substituted cycloalkyl;
R₁₁ of ULM-j is optionally substituted heterocyclyl, optionally substituted alkoxy, optionally substituted heteroaryl, optionally substituted aryl, or

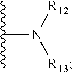

R₁₂ of ULM-j is H or optionally substituted alkyl;
R₁₃ of ULM-j is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl; optionally substituted (oxoalkyl)carbamate,
each R₁₄ of ULM-j is independently H, haloalkyl, optionally substituted cycloalkyl, optionally substituted alkyl, an azetidine, optionally substituted alkoxy, or optionally substituted heterocyclyl;
R₁₅ of ULM-j is H, CN, optionally substituted heteroaryl, haloalkyl, optionally substituted aryl, optionally substituted alkoxy, or optionally substituted heterocyclyl;
each R₁₆ of ULM-j is independently halo, optionally substituted alkyl, optionally substituted haloalkyl, optionally substituted CN, or optionally substituted haloalkoxy;
each R₂₅ of ULM-j is independently H or optionally substituted alkyl; or both R₂₅ groups can be taken together to form an oxo or optionally substituted cycloalkyl group;
R₂₃ of ULM-j is H or OH;
Z₁, Z₂, Z₃, and Z₄ of ULM-j are independently C or N; and
o of ULM-j is 0, 1, 2, 3, or 4, or a pharmaceutically acceptable salt, stereoisomer, solvate or polymorph thereof.

In certain embodiments, wherein G of ULM-j is C=J, J is O, R₇ is H, each R₁₄ is H, and o is 0.

In certain embodiments, wherein G of ULM-j is C=J, J is O, R₇ is H, each R₁₄ is H, R₁₅ is optionally substituted heteroaryl, and o is 0. In other instances, E is C═O and M is

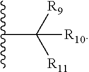

In certain embodiments, wherein E of ULM-j is C═O, R₁₁ is optionally substituted heterocyclyl or

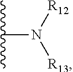

### and M is

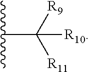

In certain embodiments, wherein E of ULM-j is C═O, M is

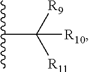

### and R₁₁ is

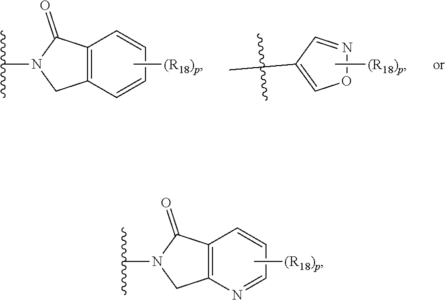

each R₁₈ is independently H, halo, optionally substituted alkoxy, cyano, optionally substituted alkyl, haloalkyl, or haloalkoxy; and p is 0, 1, 2, 3, or 4.

In certain embodiments, each R₁₄ is independently substituted with at least one of H, hydroxyl, halo, amine, amide, alkoxy, alkyl, haloalkyl, or heterocyclic.

In certain embodiments, R₁₅ of ULM-j is a group according to

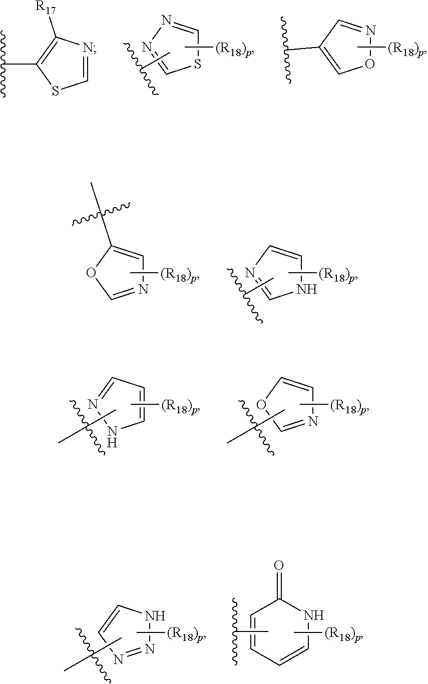

CN, or a haloalkyl, and each R₁₈ is independently H, halo, optionally substituted alkoxy, cyano, aminoalkyl, amidoalkyl, optionally substituted alkyl, haloalkyl, or haloalkoxy; and p is 0, 1, 2, 3, or 4.

In certain embodiments, ULM is a group according to the chemical structure:

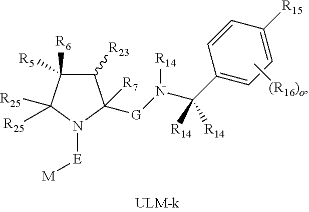

wherein:
G of ULM-k is C=J, J is O;
R₇ of ULM-k is H;
each R₁₄ of ULM-k is independently H, an amide, an alkyl, e.g., methyl, optionally substituted with one or more C₁₋₆ alkyl groups or C(O)NR′R″;
R′ and R″ are each independently H, optionally substituted alkyl, or cycloalkyl;
o of ULM-k is 0;
R₁₅ of ULM-k is defined as above for ULM-j;
R₁₆ of ULM-k is defined is as above for ULM-j; and
R₁₇ of ULM-k is H, halo, optionally substituted cycloalkyl, optionally substituted alkyl, optionally substituted alkenyl, and haloalkyl.

In other instances, R₁₇ of ULM-k is alkyl (e.g., methyl) or cycloalkyl (e.g., cyclopropyl).

In other embodiments, ULM is a group according to the chemical structure:

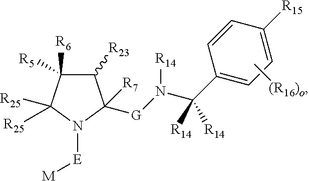

wherein:
G of ULM-k is C=J, J is O;
R₇ of ULM-k is H;
each R₁₄ of ULM-k is H;
o of ULM-k is 0; and
R₁₅ of ULM-k is selected from the group consisting of optionally substituted:

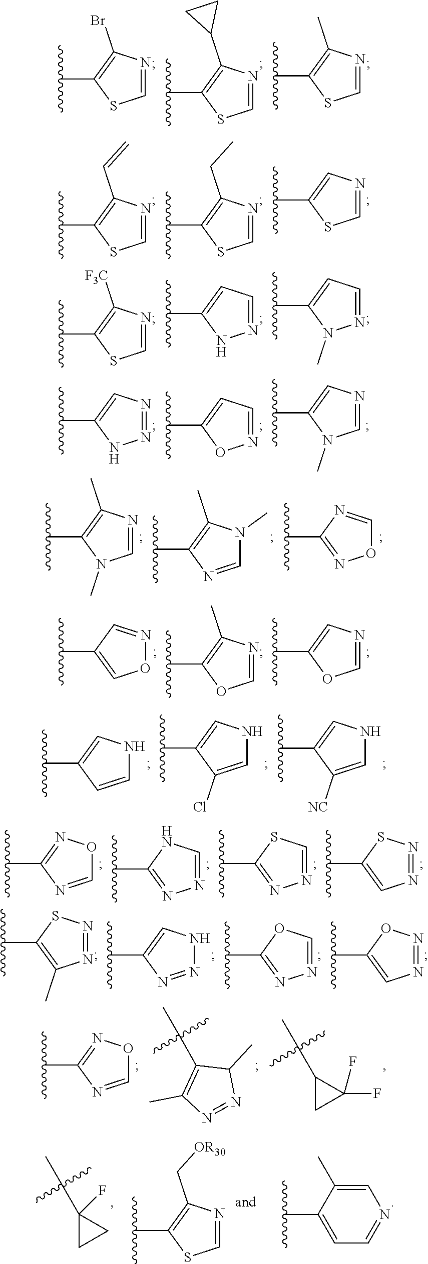

wherein R₃₀ of ULM-k is H or an optionally substituted alkyl.

In other embodiments, ULM is a group according to the chemical structure:

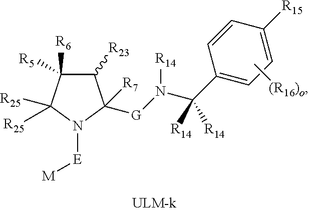

wherein:
E of ULM-k is C═O;
M of ULM-k is

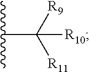

and
R₁₁ of ULM-k is selected from the group consisting of optionally substituted:

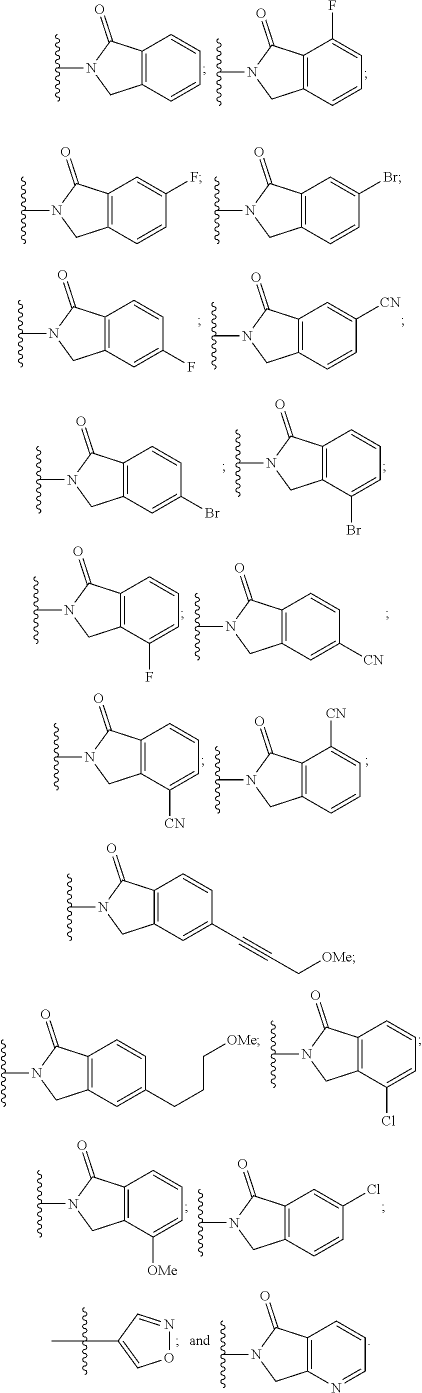

In still other embodiments, a compound of the chemical structure,

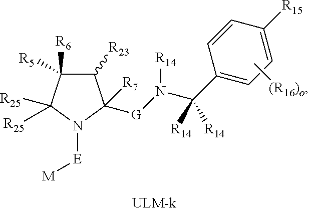

wherein:
E of ULM-k is C═O;
R₁₁ of ULM-k is

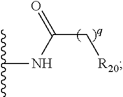

and
M of ULM-k is

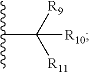

q of ULM-k is 1 or 2;
R₂₀ of ULM-k is H, optionally substituted alkyl, optionally substituted cycloalkyl, optionally substituted aryl, or

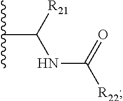

R₂₁ of ULM-k is H or optionally substituted alkyl; and
R₂₂ of ULM-k is H, optionally substituted alkyl, optionally substituted alkoxy, or haloalkyl.

In any embodiment described herein, R₁₁ of ULM-j or ULM-k is selected from the group consisting of:

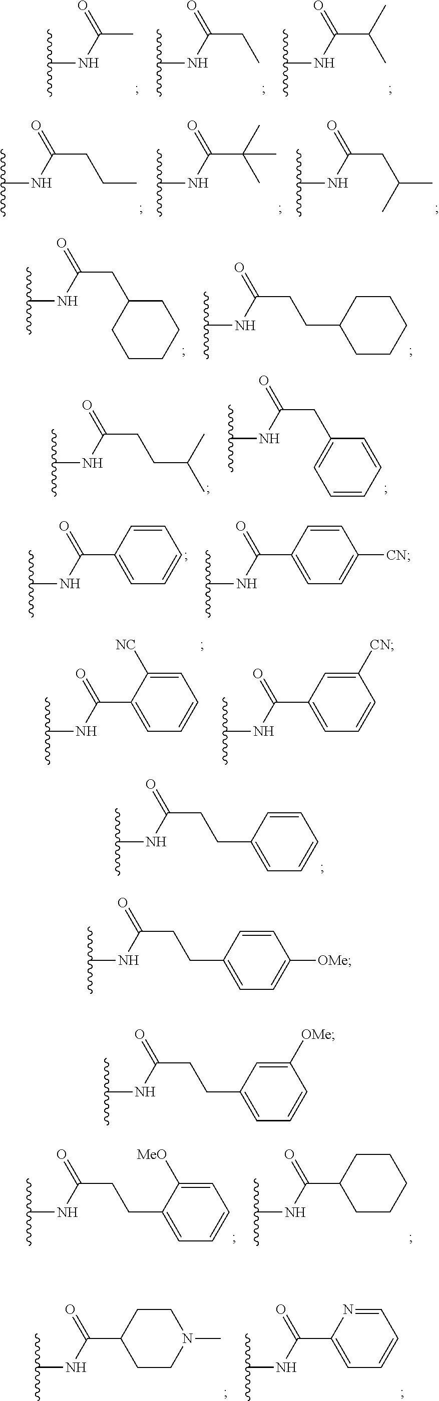

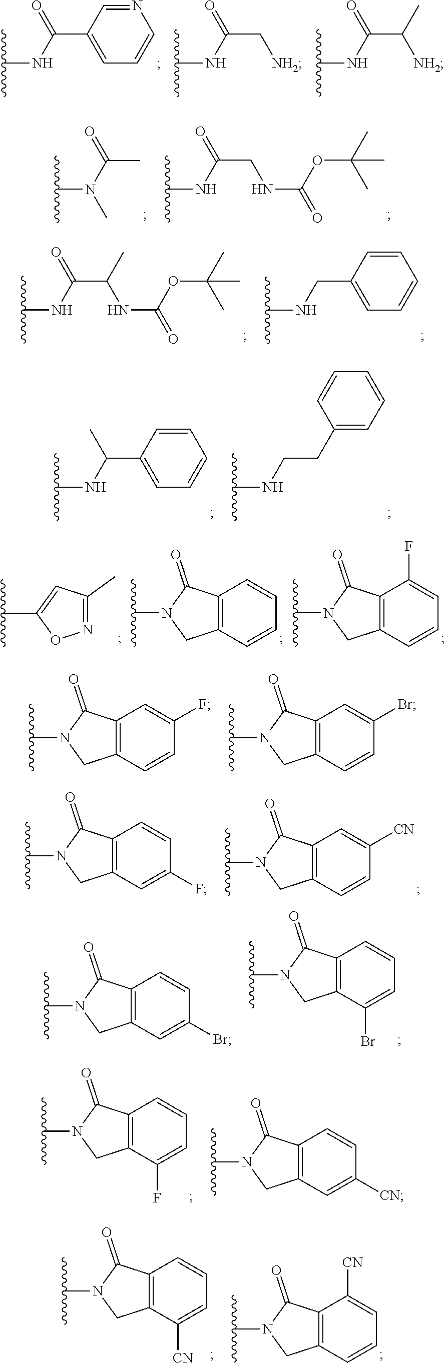

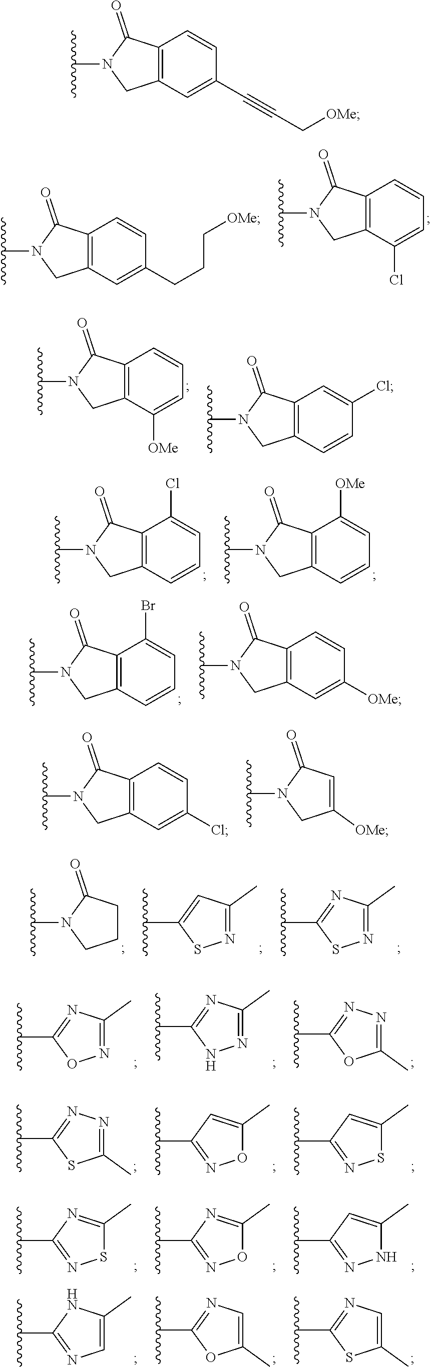

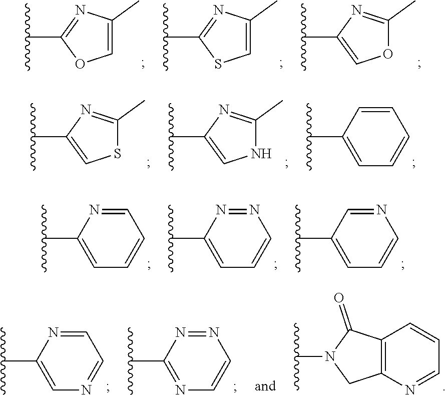

In certain embodiments, R₁₁ of ULM-j or ULM-k is selected from the group consisting of:

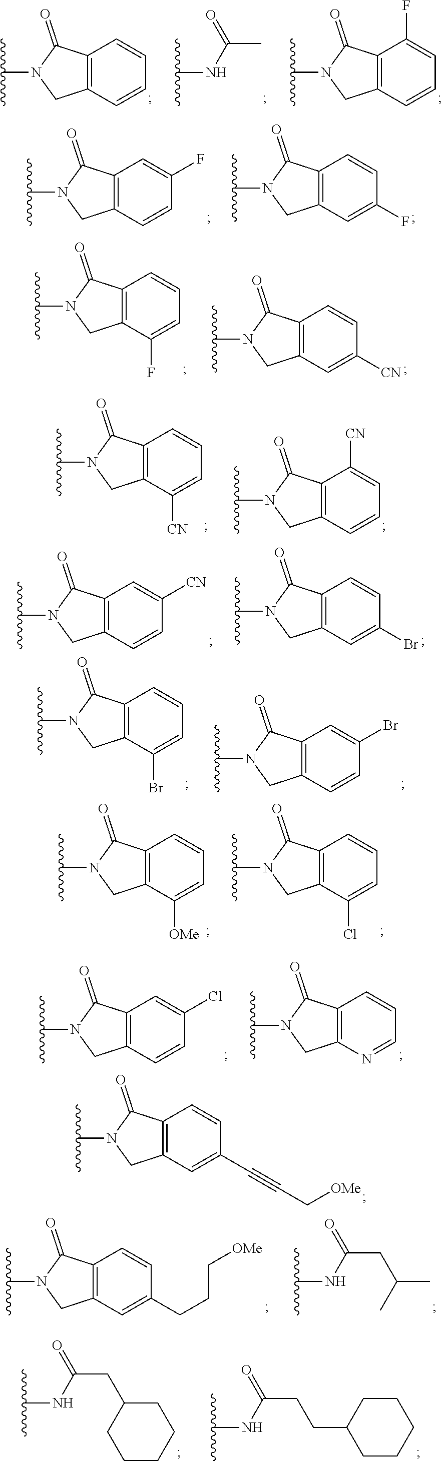

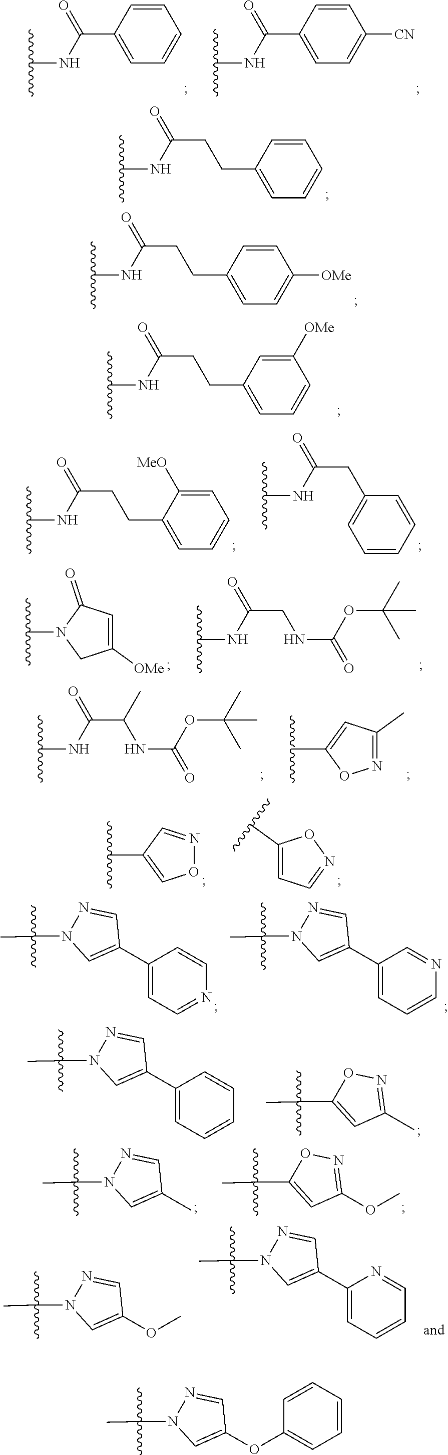

In certain embodiments ULM is a group according to the chemical structure

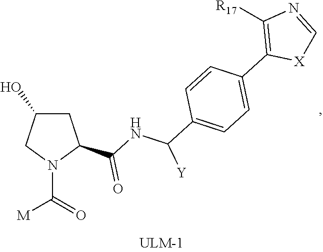

wherein:
X of ULM-l is O or S;
Y of ULM-l is H, methyl or ethyl;
R₁₇ of ULM-l is H, methyl, ethyl, hydoxymethyl or cyclopropyl;
M of ULM-l is is optionally substituted aryl, optionally substituted heteroaryl, or

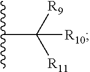

R⁹ of ULM-l is H;
R₁₀ of ULM-l is H, optionally substituted alkyl, optionally substituted haloalkyl, optionally substituted heteroaryl, optionally substituted aryl, optionally substituted hydroxyalkyl, optionally substituted thioalkyl or cycloalkyl;
R¹¹ of ULM-l is optionally substituted heteroaromatic, optionally substituted heterocyclyl, optionally substituted aryl or

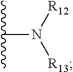

R₁₂ of ULM-l is H or optionally substituted alkyl; and
R₁₃ of ULM-l is H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl; optionally substituted (oxoalkyl)carbamate.

In some embodiments, ULM is a group according to the chemical structure:

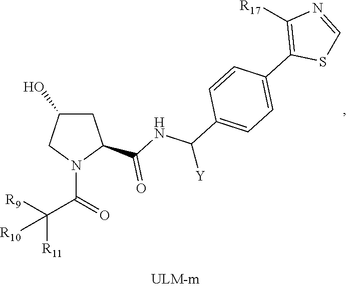

wherein:
Y of ULM-m is H, methyol or ethyl
R₉ of ULM-m is H;
R₁₀ is isopropyl, tert-butyl, sec-butyl, cyclopentyl, or cyclohexyl;
R₁₁ of ULM-m is optionally substituted amide, optionally substituted isoindolinone, optionally substituted isooxazole, optionally substituted heterocyclyls.

In other preferred embodiments of the disclosure, ULM is a group according to the chemical structure:

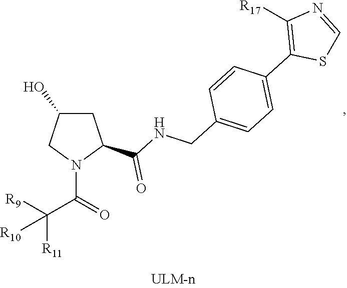

wherein:
R₁₇ of ULM-n is methyl, ethyl, or cyclopropyl; and
R₉, R₁₀, and R₁₁ of ULM-n are as defined above. In other instances, R₉ is H; and
R₁₀ of ULM-n is H, alkyl, or or cycloalkyl (preferably, isopropyl, tert-butyl, sec-butyl, cyclopentyl, or cyclohexyl).

In other preferred embodiments of the disclosure, ULM is a group according to the chemical structure:

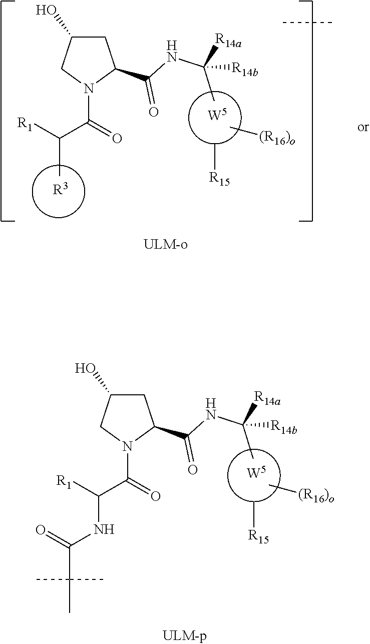

or a pharmaceutically acceptable salt thereof, wherein:
R₁ is H, optionally substituted alkyl or optionally substituted cycloalkyl;
R₃ is an optionally substituted 5-6 membered heteroaryl;
W⁵ is optionally substituted phenyl, optionally substituted napthyl or optionally substituted pyridinyl;
one of R₁₄ₐ and R_{14b} is H, optionally substituted alkyl, optionally substituted haloalkyl (e.g., fluoroalkyl), optionally substituted alkoxy, optionally substituted hydroxyl alkyl, optionally substituted alkylamine, optionally substituted heterolkyl, optionally substituted alkyl-heterocycloalkyl, optionally substituted alkoxy-heterocycloalkyl, COR₂₆, CONR₂₇ₐR_{27b}, NHCOR₂₆, or NHCH₃COR₂₆; and the other of R₁₄ₐ and R_{14b} is H; or R₁₄ₐ, R_{14b}, together with the carbon atom to which they are attached, form an optionally substituted 3 to 6 membered cycloalkyl, heterocycloalkyl, spirocycloalkyl or spiroheterocyclyl, wherein the spiroheterocyclyl is not epoxide or aziridine;
R₁₅ is CN, optionally substituted fluoroalkyl,

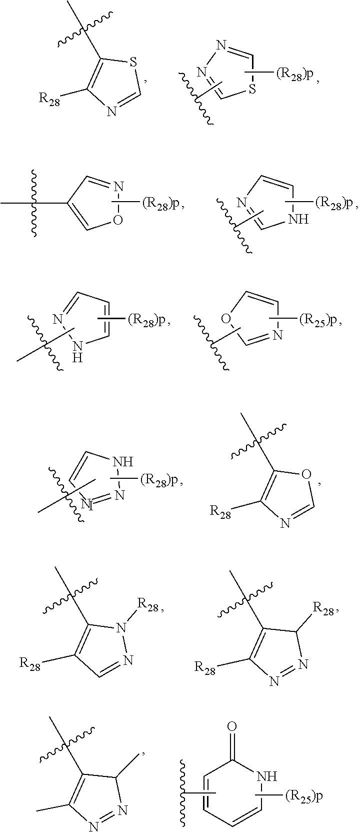

optionally substituted

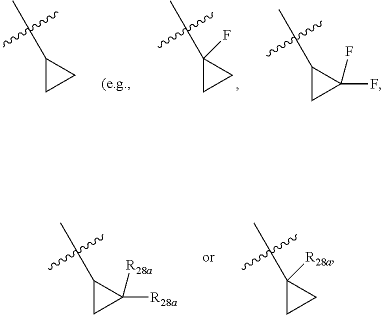

wherein R₂₈ₐ is halo, optionally substituted alkyl or fluoroalkyl, or

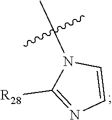

each R₁₆ is independently selected from halo, CN, optionally substituted alkyl, optionally substituted haloalkyl, hydroxy, or haloalkoxy;
each R₂₆ is independently H, optionally substituted alkyl or NR₂₇ₐR_{27b};
each R₂₇ₐ and R_{27b} is independently H, optionally substituted alkyl, optionally substituted cycloalkyl (e.g. optionally substituted 3-5 member cycloalkyl), or R₂₇ₐ and R_{27b} together with the nitrogen atom to which they are attached form a 4-6 membered heterocyclyl;
each R₂₈ is independently H, halogen, CN, optionally substituted aminoalkyl, optionally substituted amidoalkyl, optionally substituted haloalkyl, optionally substituted alkyl, optionally substituted alkoxy, optionally substituted heteroalkyl, optionally substituted alkylamine, optionally substituted hydroxyalkyl, amine, optionally substituted alkynyl, or optionally substituted cycloalkyl;
o is 0, 1 or 2; and
p is 0, 1, 2, 3, or 4.

In any of the aspects or embodiments described herein, the ULM is of the formula:

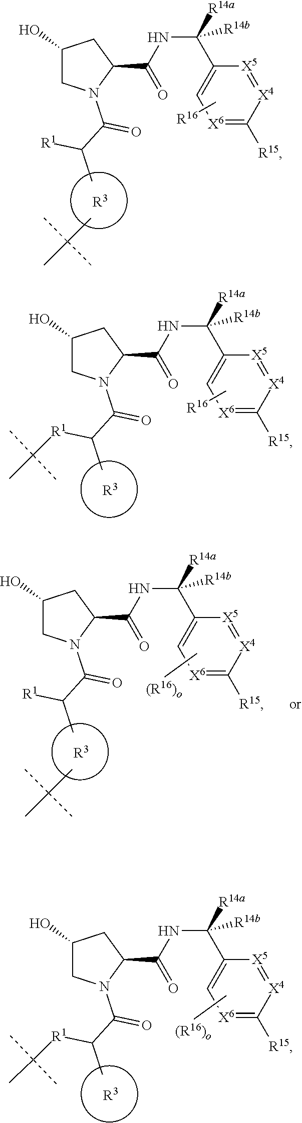

wherein:
each of X⁴, X⁵, and X⁶ is selected from CH and N, wherein no more than 2 are N;
R¹ is C₁₋₆ alkyl;
R³ is the same as defined for ULM-o and ULM-p
one of R^{14a} and R^{14b} is H, optionally substituted alkyl, optionally substituted haloalkyl, optionally substituted alkoxy, optionally substituted hydroxyl alkyl, optionally substituted alkylamine, optionally substituted amide, optionally substituted alkyl-amide, optionally substituted alkyl-cyano, optionally substituted alkyl-phosphate, optionally substituted heteraolkyl, optionally substituted alkyl-heterocycloalkyl, optionally substituted alkoxy-heterocycloalkyl, COR²⁶, CONR^{27a}R^{27b}, NHCOR²⁶, or NHCH₃COR²⁶; and the other of R^{14a} and R^{14b} is H; or R^{14a} and R^{14b}, together with the carbon atom to which they are attached, form an optionally substituted 3 to 5 membered cycloalkyl, heterocycloalkyl, spirocycloalkyl or spiroheterocyclyl, wherein the spiroheterocyclyl is not epoxide or aziridine;
each R₂₇ₐ and R_{27b} is independently H C₁₋₆ alkyl or cyclolkyl (e.g., optionally substituted 3-5 member cycloalkyl);
o is 0, 1, or 2;
q is 1, 2, 3 or 4;
R¹⁵ is optionally substituted

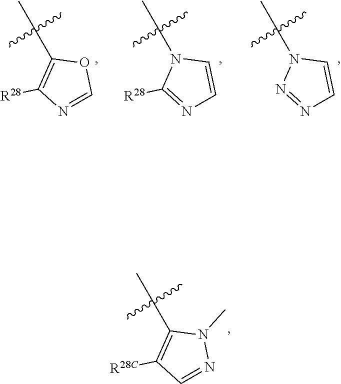

### or CN;

R²⁸ is H, methyl, CH₂N(Me)₂, CH₂OH, CH₂O(C₁₋₄alkyl), CH₂NHC(O)C₁₋₄alkyl, NH₂,

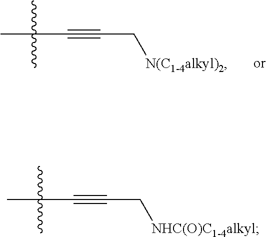

R^{28C} is H, methyl, fluoro, or chloro; and
R¹⁶ is H, C₁₋₄alkyl, fluoro, chloro, CN, or C₁₋₄alkoxy.

In any aspect or embodiment described herein, R^{14a} and R^{14b} are selected from: H, C₁₋₄ alkyl, C₁₋₄ cycloalkyl, C₁₋₄haloalkyl, C₁₋₄ hydroxyalkyl, C₁₋₄ alkyloxyalkyl, C₁₋₄ alkyl-NR₂₇ₐR_{27b} and CONR₂₇ₐR_{27b}.

In any aspect or embodiment described herein, at least one of R^{14a} and R^{14b} is H (e.g., both R^{14a} and R^{14b} are H).

In any aspect or embodiment described herein, at least one of R^{14a} and R^{14b} is optionally substituted alkyl, optionally substituted haloalkyl, optionally substituted alkoxy, optionally substituted hydroxyl alkyl, optionally substituted alkylamine, optionally substituted heterolkyl, optionally substituted alkyl-heterocycloalkyl, optionally substituted alkoxy-heterocycloalkyl, COR²⁶, CONR^{27a}R^{27b}, NHCOR²⁶, or NHCH₃COR²⁶. Alternatively, in any aspect or embodiment described herein, one of R^{14a} and R^{14b} is optionally substituted alkyl, optionally substituted haloalkyl, optionally substituted alkoxy, optionally substituted hydroxyl alkyl, optionally substituted alkylamine, optionally substituted heterolkyl, optionally substituted alkyl-heterocycloalkyl, optionally substituted alkoxy-heterocycloalkyl, COR²⁶, CONR^{27a}R^{27b}, NHCOR²⁶, or NHCH₃COR²⁶; and the other of R^{14a} and R^{14b} is H.

In any aspect or embodiment described herein, R^{14a} and R^{14b} together with the carbon atom to which they are attached form

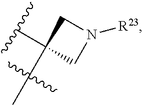

wherein R²³ is selected from H, C₁₋₄alkyl, —C(O)C₁₋₄alkyl.

In other preferred embodiments of the disclosure, ULM is a group according to the chemical structure:

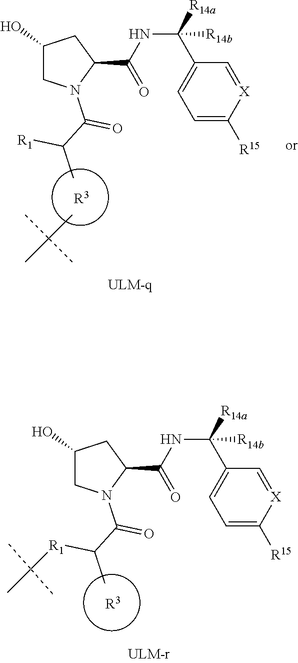

or a pharmaceutically acceptable salt thereof, wherein:
X is CH or N; and
R₁, R₃, R₁₄ₐ, R_{14b}, and R₁₅ of ULM-q and ULM-r are the same as defined for ULM-o and ULM-p.

In any of the aspects or embodiments described herein, the ULM as described herein may be a pharmaceutically acceptable salt, enantiomer, diastereomer, solvate or polymorph thereof. In addition, in any of the aspects or embodiments described herein, the ULM as described herein may be coupled to a PTM directly via a bond or by a chemical linker.

In certain aspects of the disclosure, the ULM moiety is selected from the group consisting of:

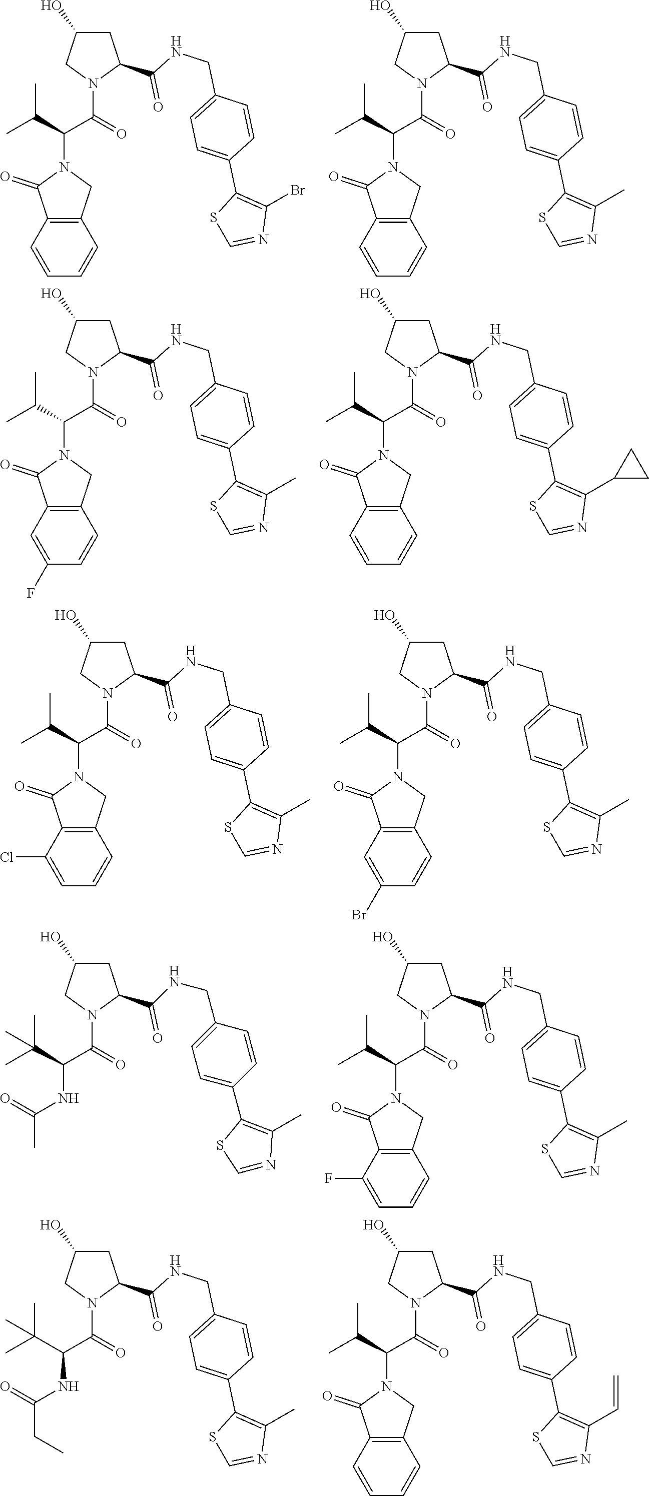

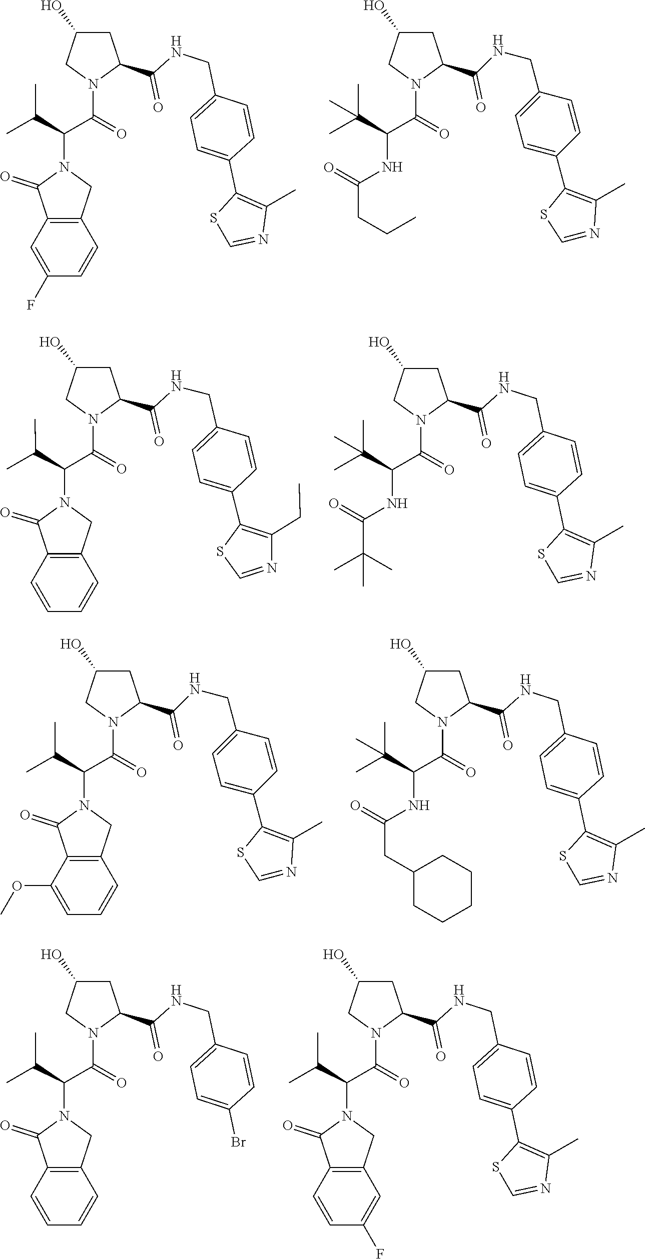

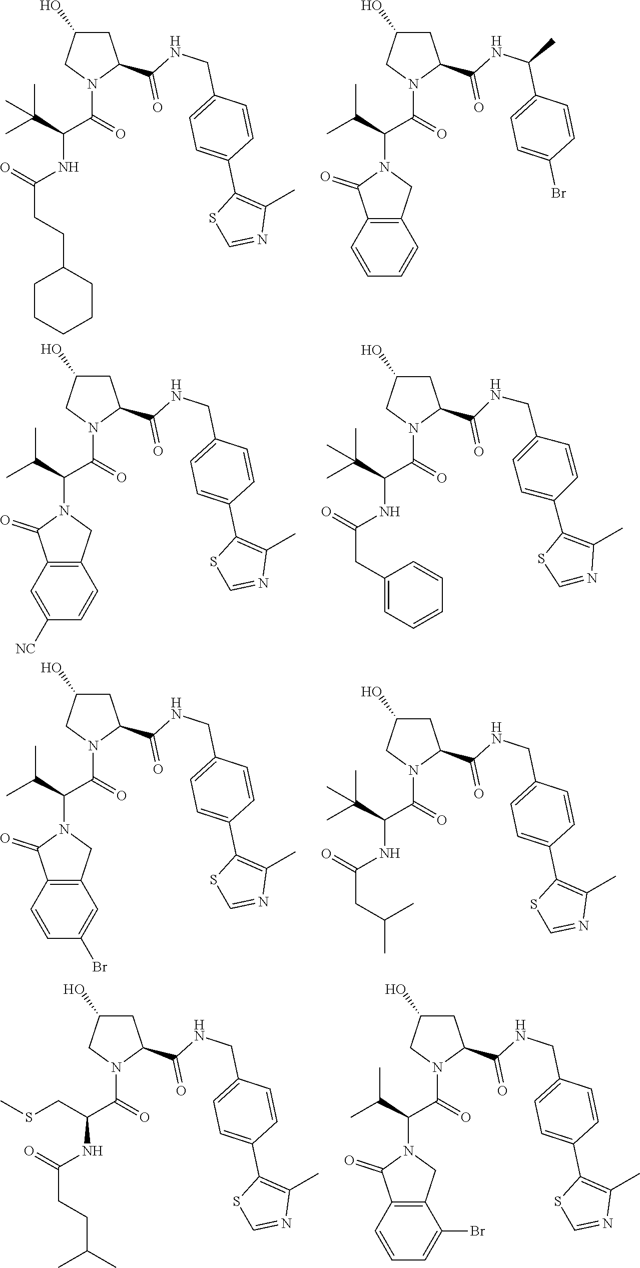

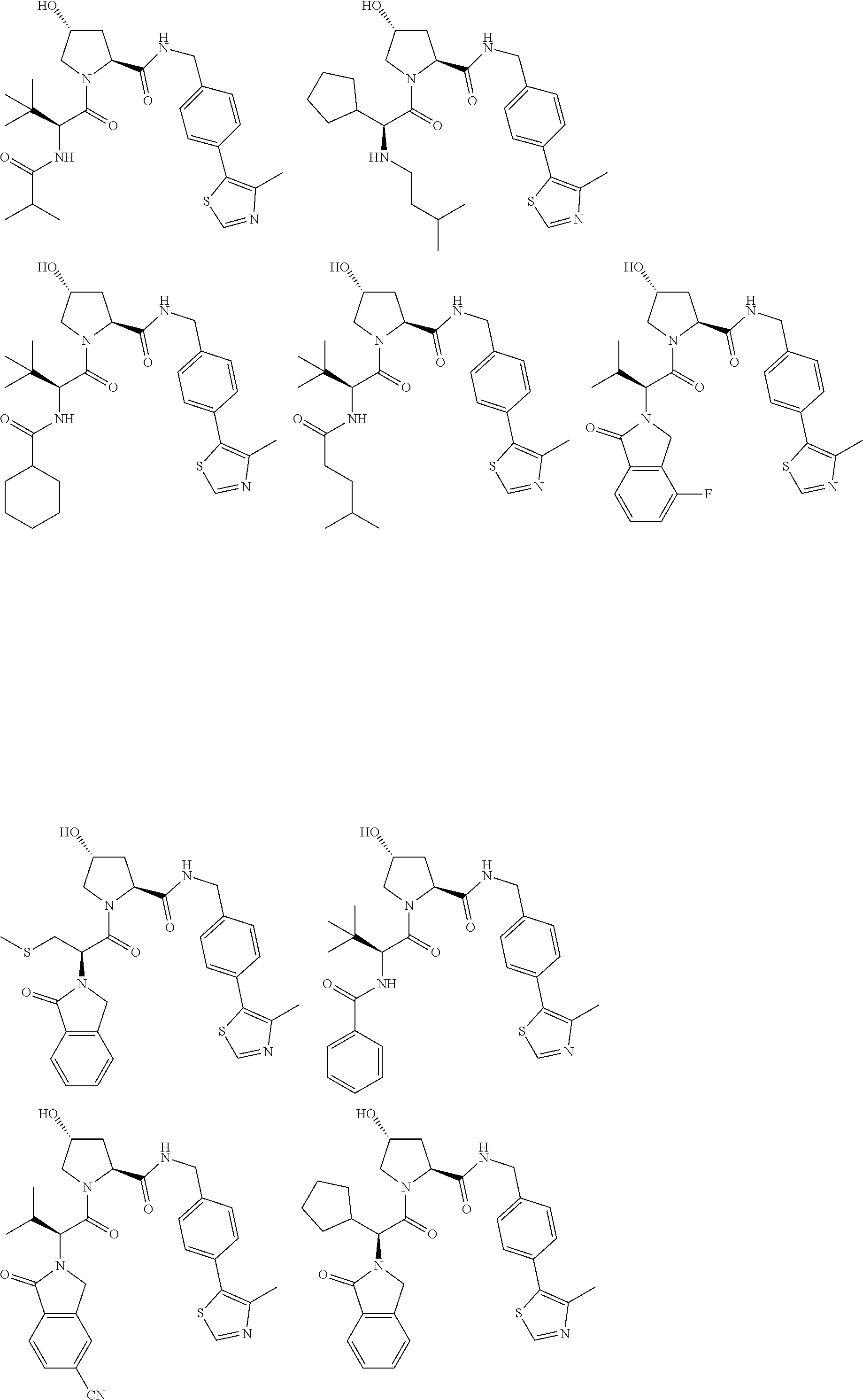

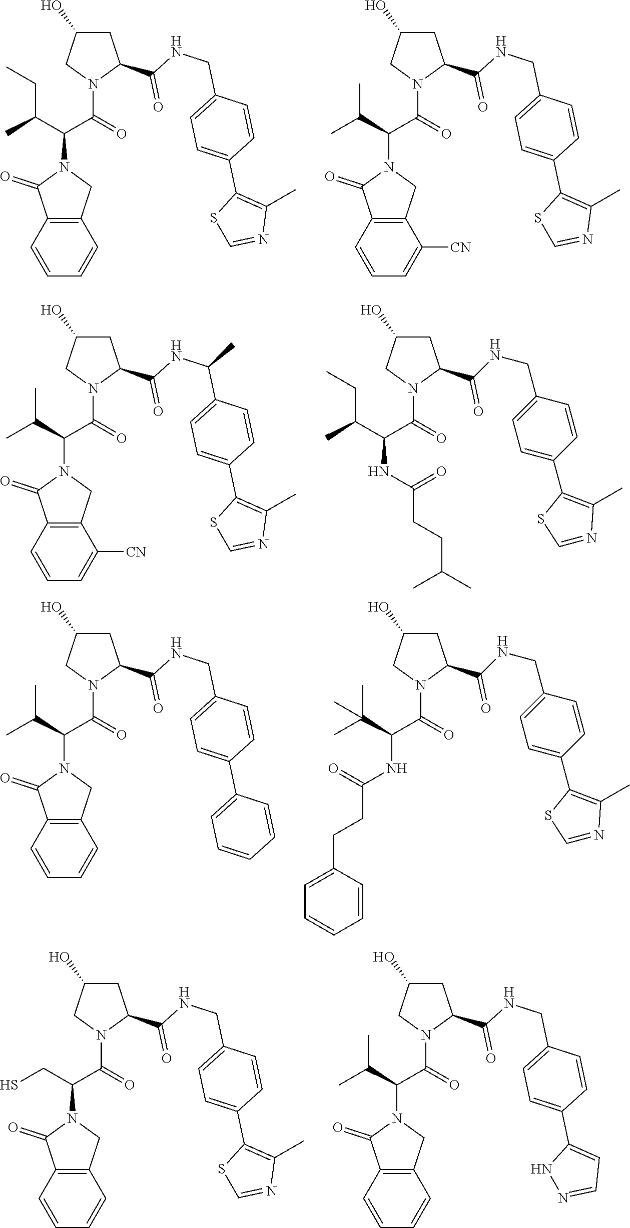

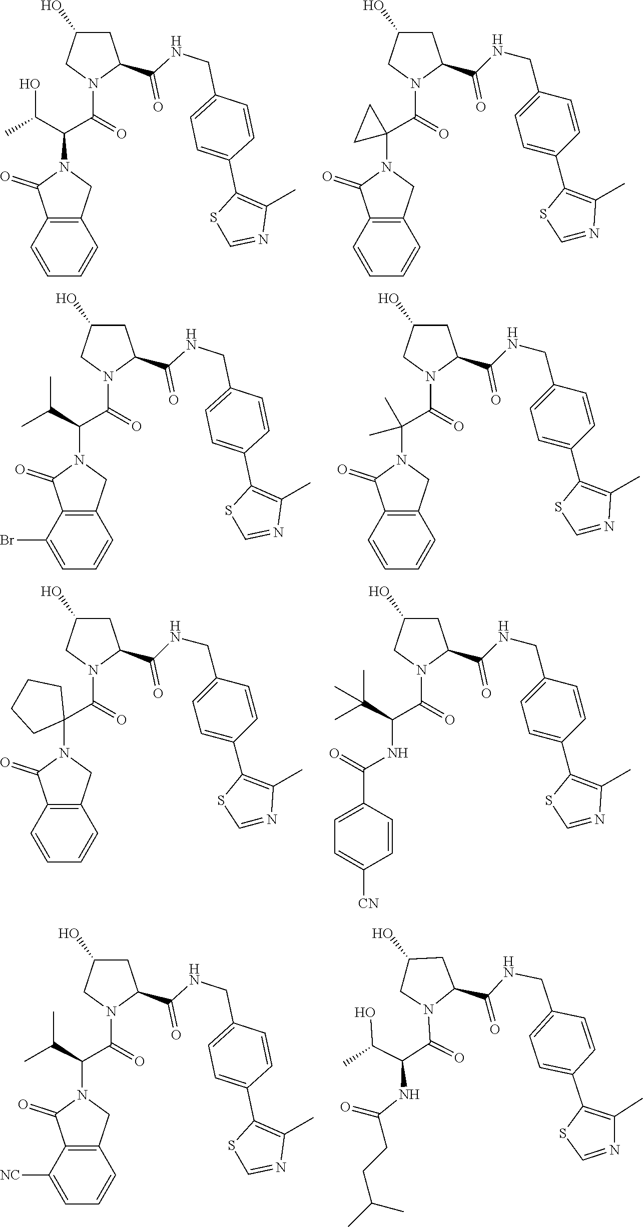

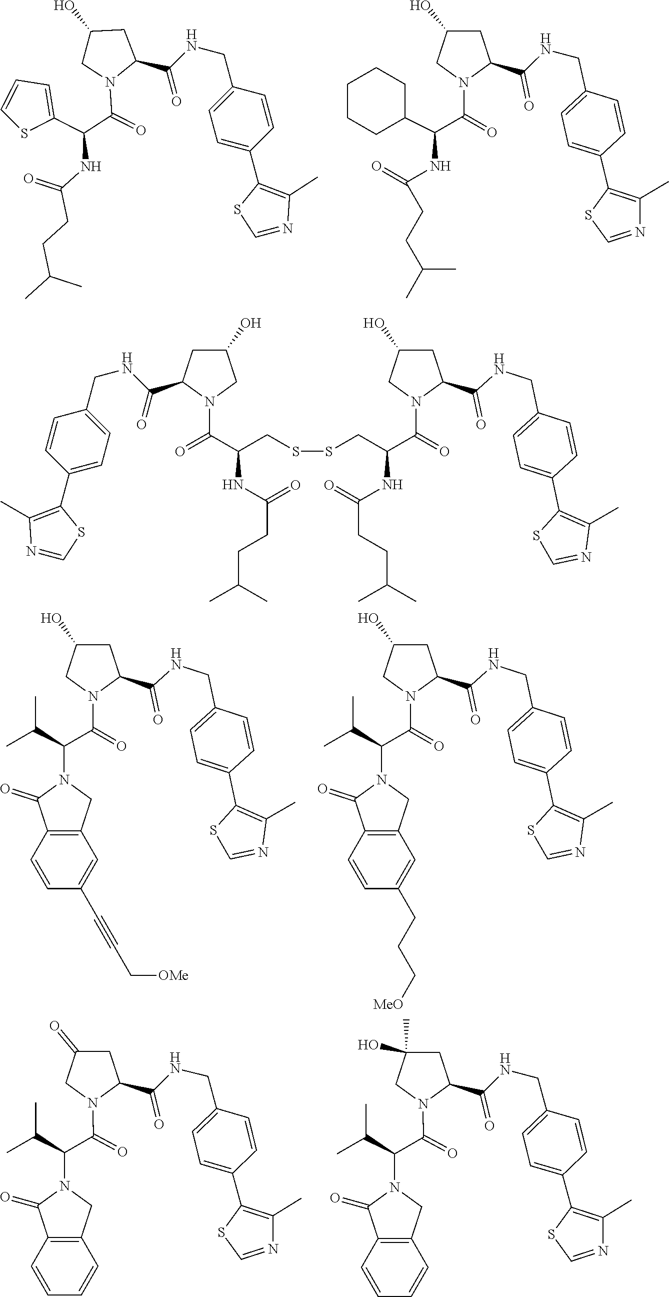

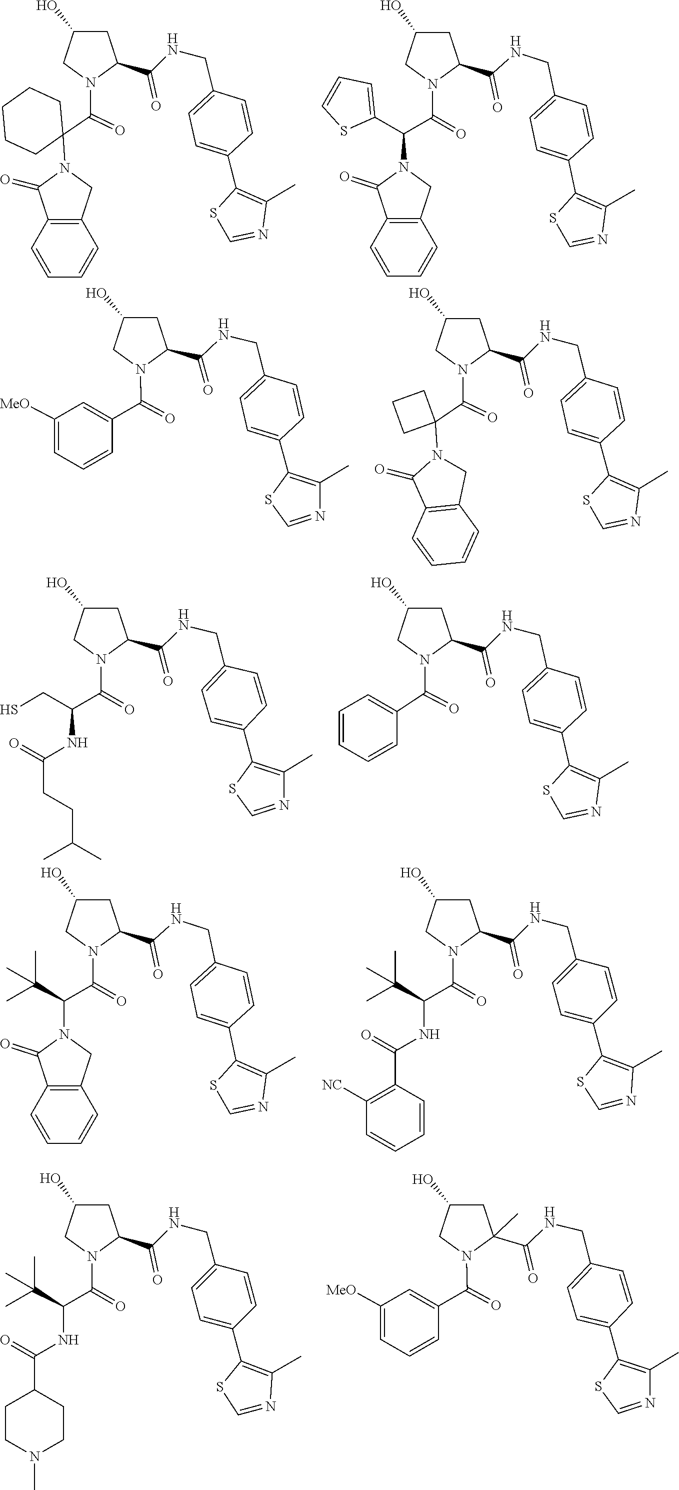

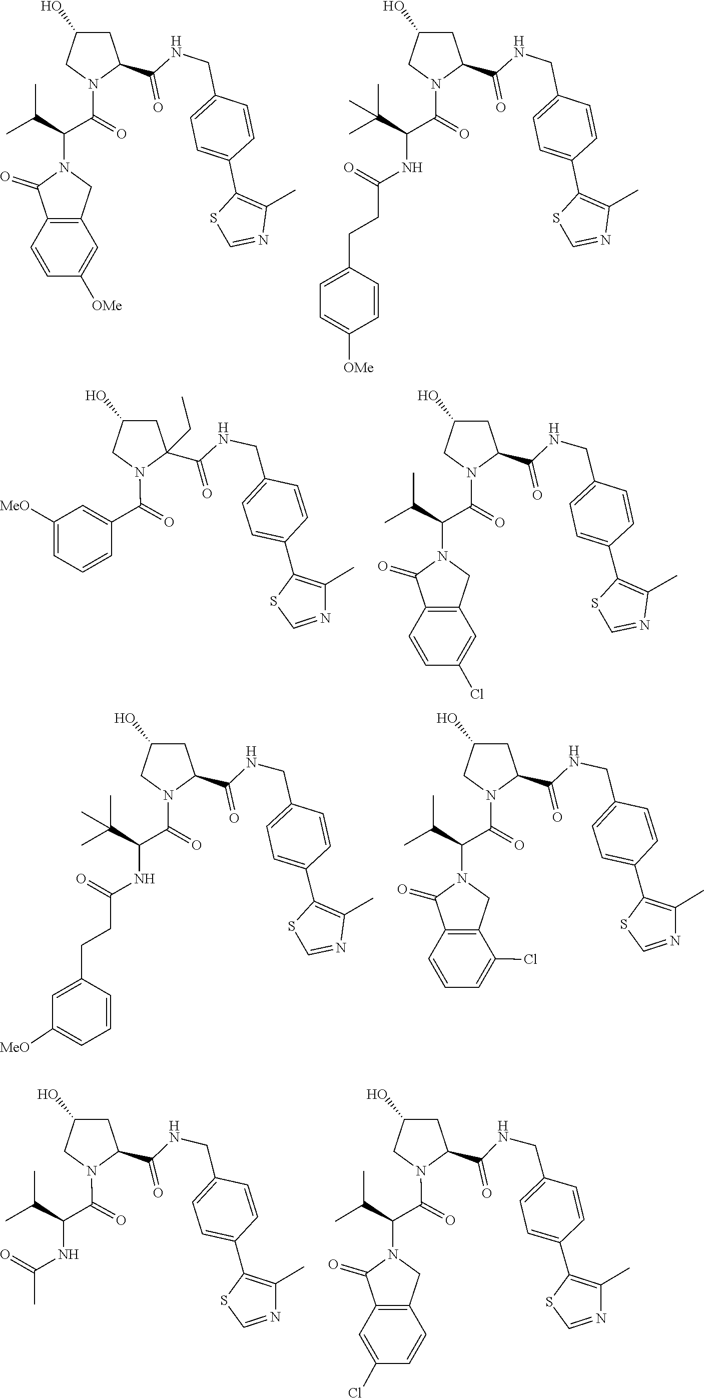

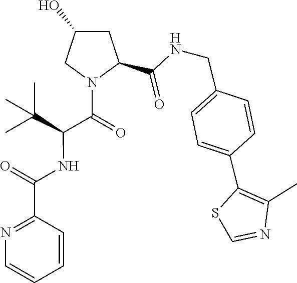 [SMILES: Cc1ncsc1-c1ccc(CNC(=O)[C@@H]2C[C@@H](O)CN2C(=O)[C@@H](NC(=O)c2ccccn2)C(C)(C)C)cc1]

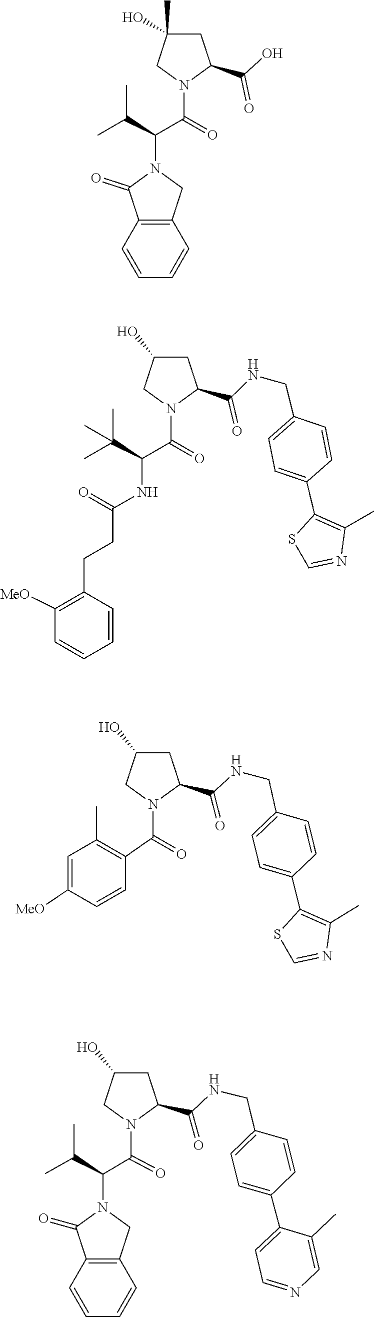 [SMILES: CC(C)[C@@H](C(=O)N1C[C@](C)(O)C[C@H]1C(=O)O)N1Cc2ccccc2C1=O ; COc1ccccc1CCC(=O)N[C@H](C(=O)N1C[C@H](O)C[C@H]1C(=O)NCc1ccc(-c2scnc2C)cc1)C(C)(C)C ; COc1ccc(C(=O)N2C[C@H](O)C[C@H]2C(=O)NCc2ccc(-c3scnc3C)cc2)c(C)c1 ; Cc1cnccc1-c1ccc(CNC(=O)[C@@H]2C[C@@H](O)CN2C(=O)[C@H](C(C)C)N2Cc3ccccc3C2=O)cc1]

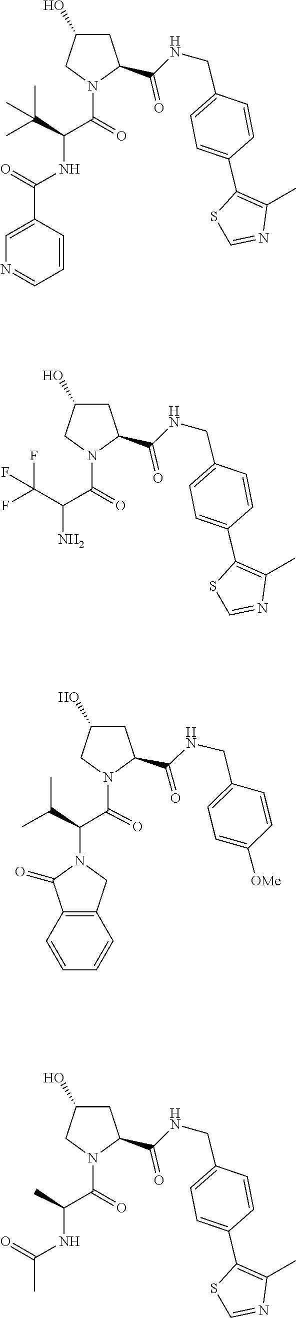 [SMILES: Cc1ncsc1-c1ccc(CNC(=O)[C@@H]2C[C@@H](O)CN2C(=O)[C@@H](NC(=O)c2cccnc2)C(C)(C)C)cc1 ; Cc1ncsc1-c1ccc(CNC(=O)[C@@H]2C[C@@H](O)CN2C(=O)C(N)C(F)(F)F)cc1 ; COc1ccc(CNC(=O)[C@@H]2C[C@@H](O)CN2C(=O)[C@H](C(C)C)N2Cc3ccccc3C2=O)cc1 ; CC(=O)N[C@@H](C)C(=O)N1C[C@H](O)C[C@H]1C(=O)NCc1ccc(-c2scnc2C)cc1]

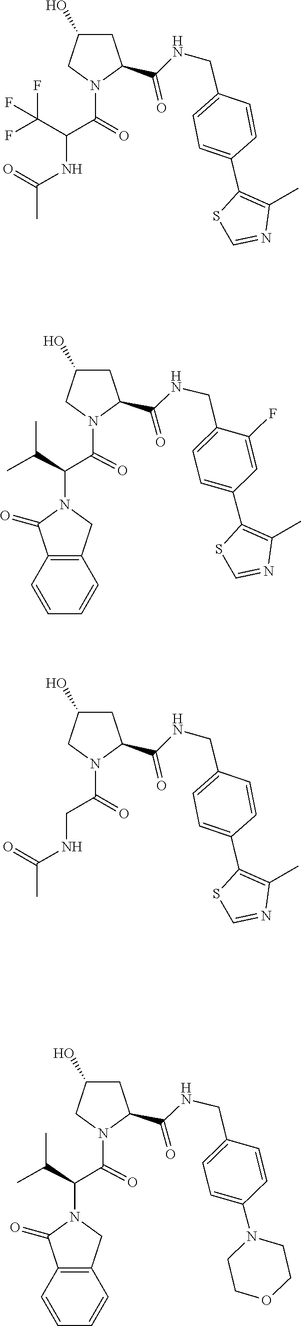 [SMILES: CC(=O)NC(C(=O)N1C[C@H](O)C[C@H]1C(=O)NCc1ccc(-c2scnc2C)cc1)C(F)(F)F ; Cc1ncsc1-c1ccc(CNC(=O)[C@@H]2C[C@@H](O)CN2C(=O)[C@H](C(C)C)N2Cc3ccccc3C2=O)c(F)c1 ; CC(=O)NCC(=O)N1C[C@H](O)C[C@H]1C(=O)NCc1ccc(-c2scnc2C)cc1 ; CC(C)[C@@H](C(=O)N1C[C@H](O)C[C@H]1C(=O)NCc1ccc(N2CCOCC2)cc1)N1Cc2ccccc2C1=O]

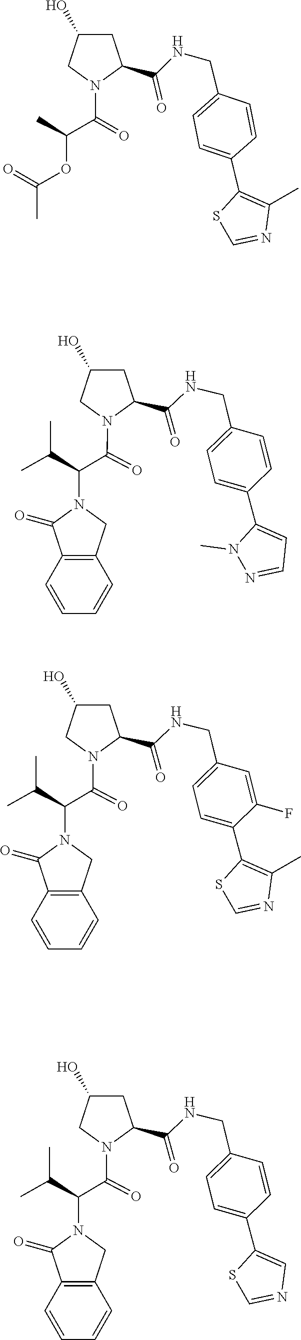 [SMILES: CC(=O)O[C@@H](C)C(=O)N1C[C@H](O)C[C@H]1C(=O)NCc1ccc(-c2scnc2C)cc1 ; CC(C)[C@@H](C(=O)N1C[C@H](O)C[C@H]1C(=O)NCc1ccc(-c2ccnn2C)cc1)N1Cc2ccccc2C1=O ; Cc1ncsc1-c1ccc(CNC(=O)[C@@H]2C[C@@H](O)CN2C(=O)[C@H](C(C)C)N2Cc3ccccc3C2=O)cc1F ; CC(C)[C@@H](C(=O)N1C[C@H](O)C[C@H]1C(=O)NCc1ccc(-c2cncs2)cc1)N1Cc2ccccc2C1=O]

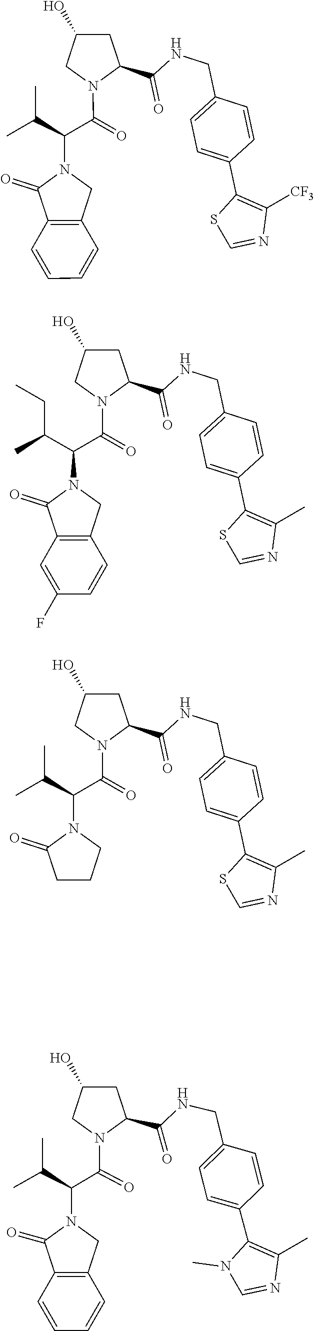

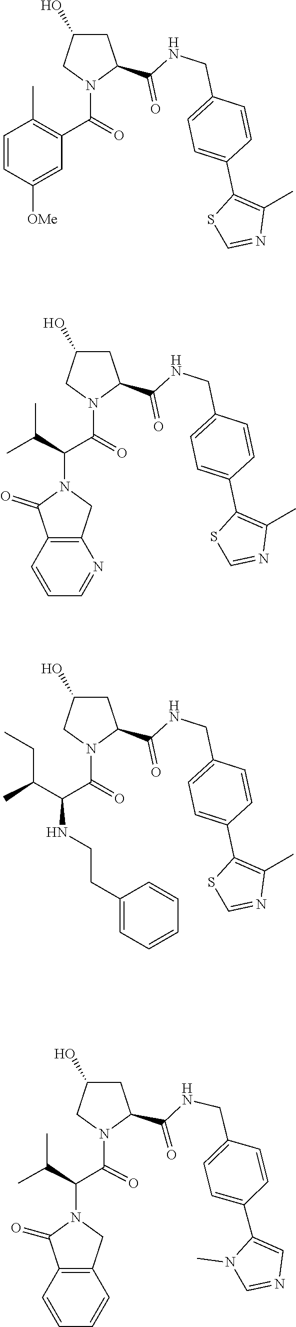

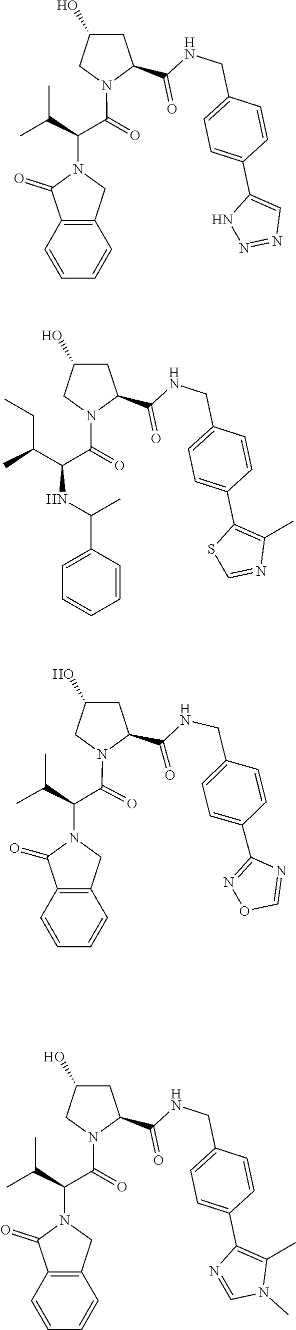 [SMILES: CC(C)[C@@H](C(=O)N1C[C@H](O)C[C@H]1C(=O)NCc1ccc(-c2cnn[nH]2)cc1)N1Cc2ccccc2C1=O ; CC[C@H](C)[C@H](NC(C)c1ccccc1)C(=O)N1C[C@H](O)C[C@H]1C(=O)NCc1ccc(-c2scnc2C)cc1 ; CC(C)[C@@H](C(=O)N1C[C@H](O)C[C@H]1C(=O)NCc1ccc(-c2ncon2)cc1)N1Cc2ccccc2C1=O ; Cc1c(-c2ccc(CNC(=O)[C@@H]3C[C@@H](O)CN3C(=O)[C@H](C(C)C)N3Cc4ccccc4C3=O)cc2)ncn1C]

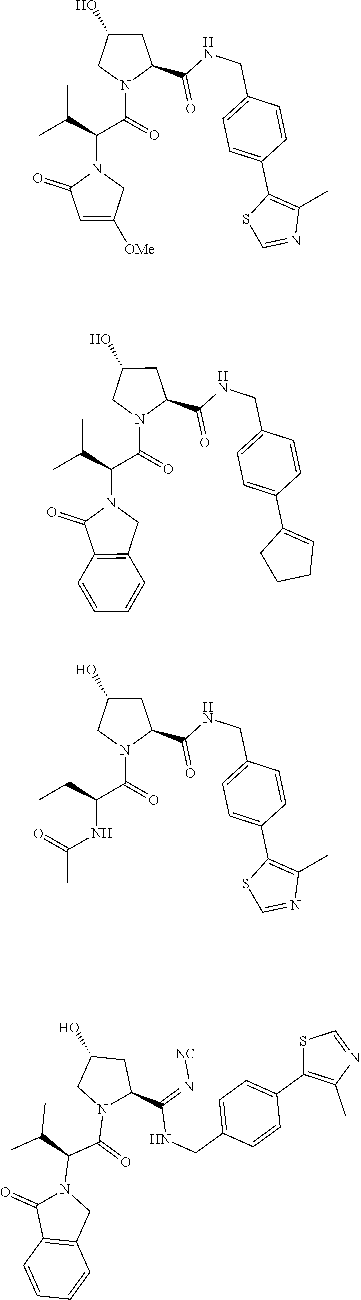

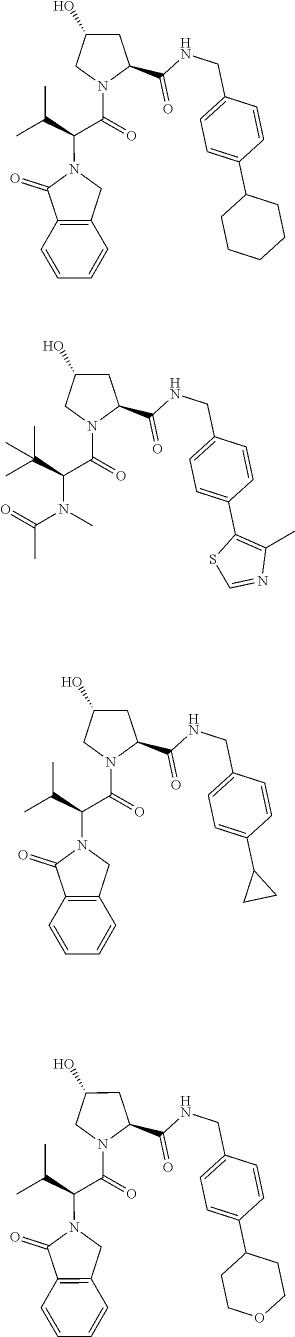 [SMILES: CC(C)[C@@H](C(=O)N1C[C@H](O)C[C@H]1C(=O)NCc1ccc(C2CCCCC2)cc1)N1Cc2ccccc2C1=O ; CC(=O)N(C)[C@H](C(=O)N1C[C@H](O)C[C@H]1C(=O)NCc1ccc(-c2scnc2C)cc1)C(C)(C)C ; CC(C)[C@@H](C(=O)N1C[C@H](O)C[C@H]1C(=O)NCc1ccc(C2CC2)cc1)N1Cc2ccccc2C1=O ; CC(C)[C@@H](C(=O)N1C[C@H](O)C[C@H]1C(=O)NCc1ccc(C2CCOCC2)cc1)N1Cc2ccccc2C1=O]

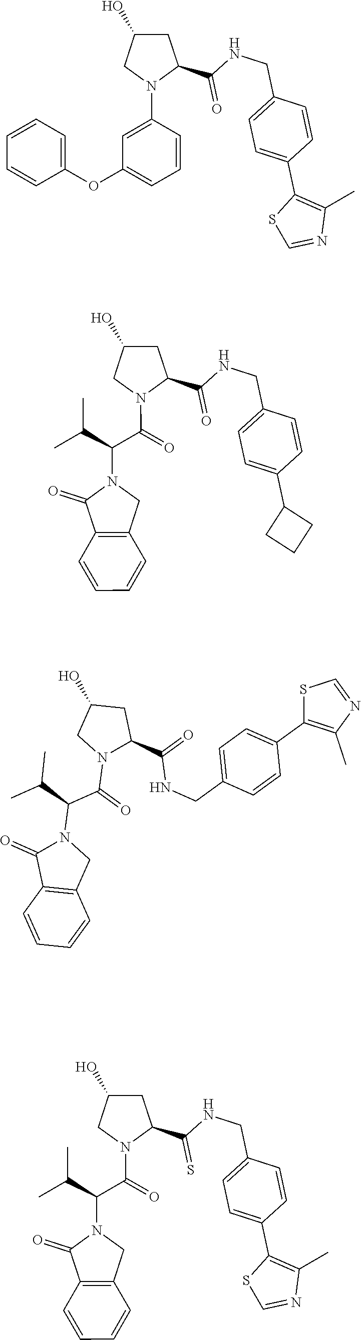 [SMILES: Cc1ncsc1-c1ccc(CNC(=O)[C@@H]2C[C@@H](O)CN2c2cccc(Oc3ccccc3)c2)cc1 ; CC(C)[C@@H](C(=O)N1C[C@H](O)C[C@H]1C(=O)NCc1ccc(C2CCC2)cc1)N1Cc2ccccc2C1=O ; Cc1ncsc1-c1ccc(CNC(=O)[C@@H]2C[C@@H](O)CN2C(=O)[C@H](C(C)C)N2Cc3ccccc3C2=O)cc1 ; Cc1ncsc1-c1ccc(CNC(=S)[C@@H]2C[C@@H](O)CN2C(=O)[C@H](C(C)C)N2Cc3ccccc3C2=O)cc1]

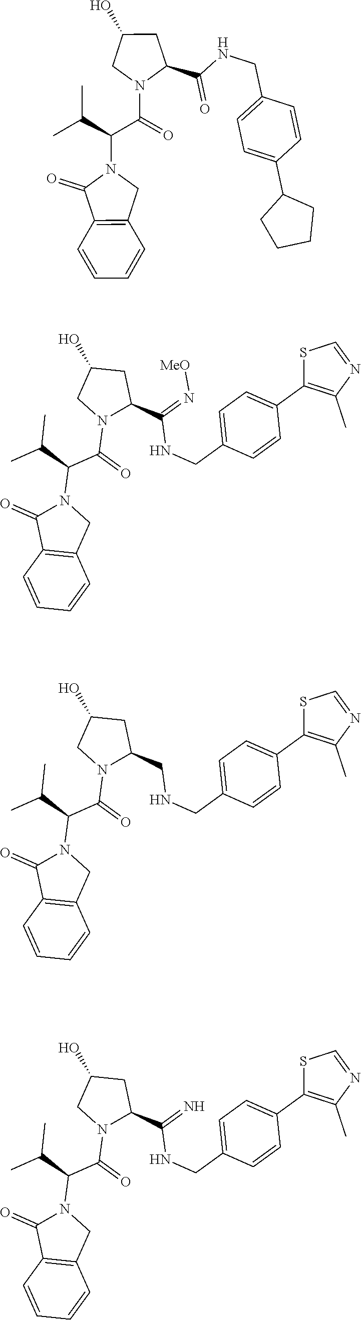 [SMILES: CC(C)[C@@H](C(=O)N1C[C@H](O)C[C@H]1C(=O)NCc1ccc(C2CCCC2)cc1)N1Cc2ccccc2C1=O ; CO/N=C(/NCc1ccc(-c2scnc2C)cc1)[C@@H]1C[C@@H](O)CN1C(=O)[C@H](C(C)C)N1Cc2ccccc2C1=O ; Cc1ncsc1-c1ccc(CNC[C@@H]2C[C@@H](O)CN2C(=O)[C@H](C(C)C)N2Cc3ccccc3C2=O)cc1 ; Cc1ncsc1-c1ccc(CNC(=N)[C@@H]2C[C@@H](O)CN2C(=O)[C@H](C(C)C)N2Cc3ccccc3C2=O)cc1]

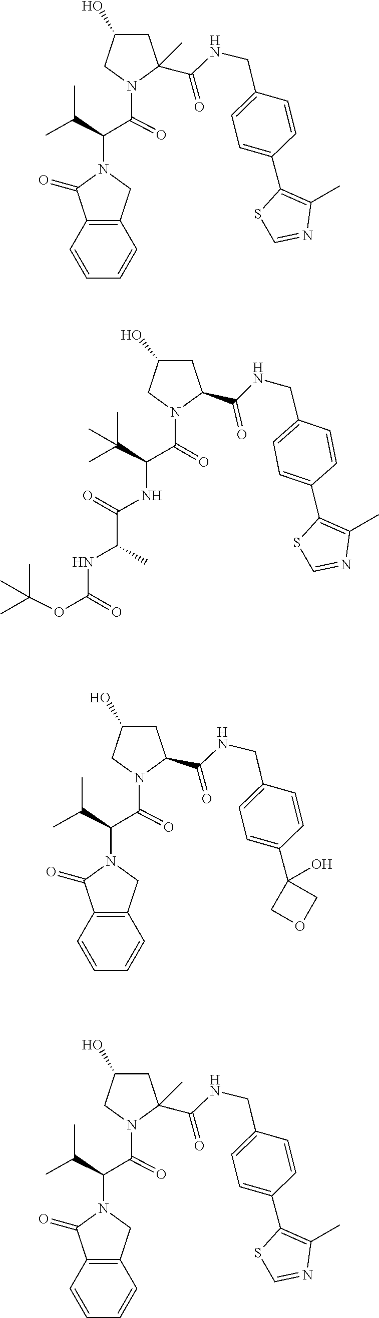

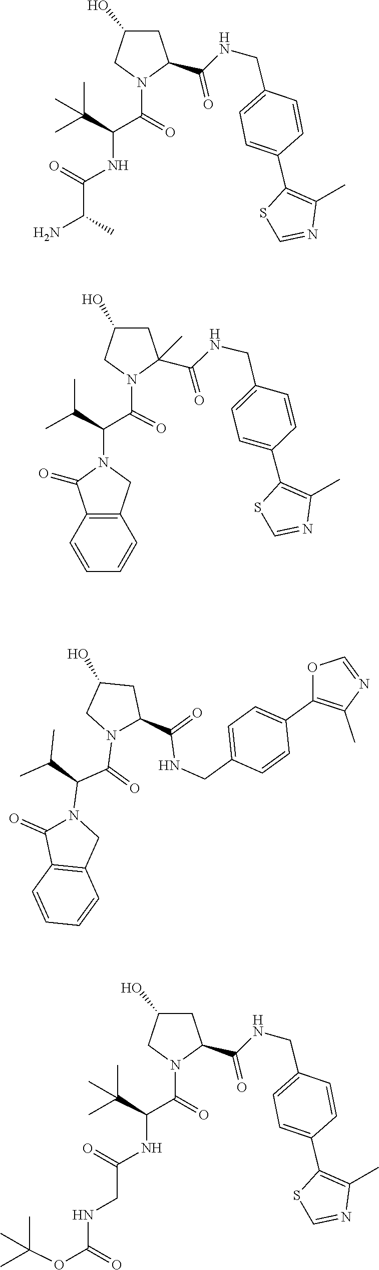

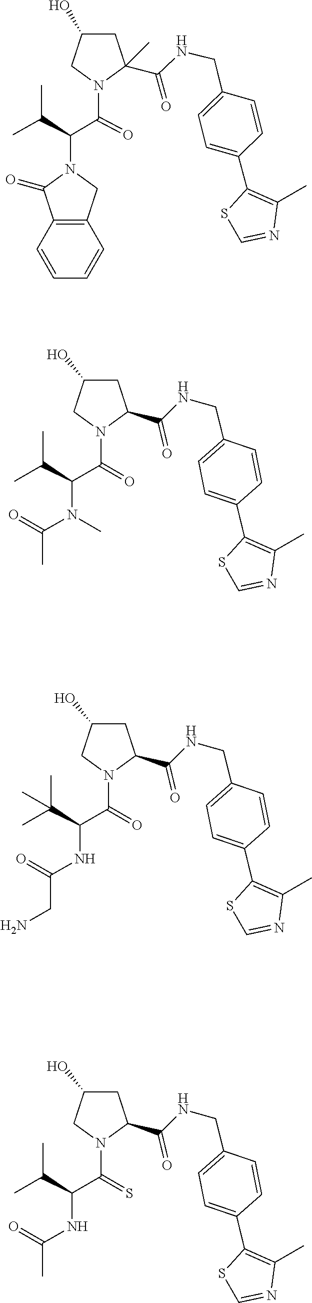 [SMILES: Cc1ncsc1-c1ccc(CNC(=O)C2(C)C[C@@H](O)CN2C(=O)[C@H](C(C)C)N2Cc3ccccc3C2=O)cc1 ; CC(=O)N(C)[C@H](C(=O)N1C[C@H](O)C[C@H]1C(=O)NCc1ccc(-c2scnc2C)cc1)C(C)C ; Cc1ncsc1-c1ccc(CNC(=O)[C@@H]2C[C@@H](O)CN2C(=O)[C@@H](NC(=O)CN)C(C)(C)C)cc1 ; CC(=O)N[C@H](C(=S)N1C[C@H](O)C[C@H]1C(=O)NCc1ccc(-c2scnc2C)cc1)C(C)C]

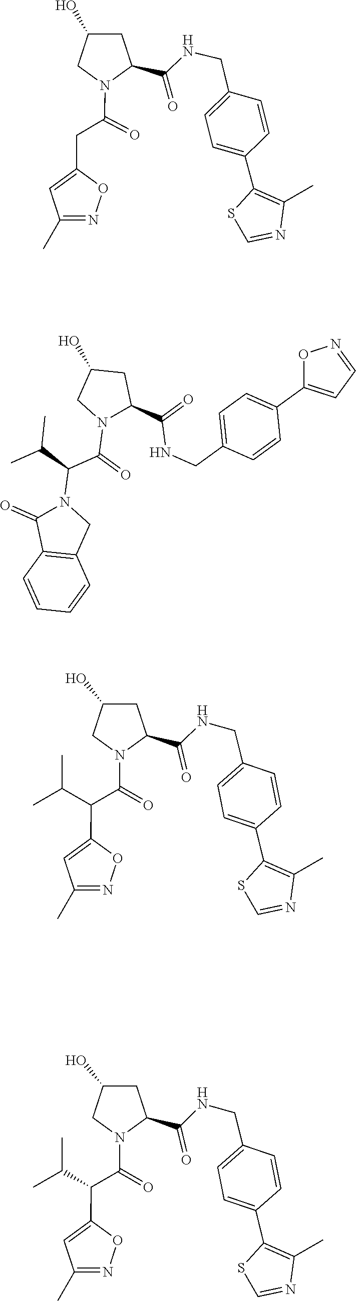

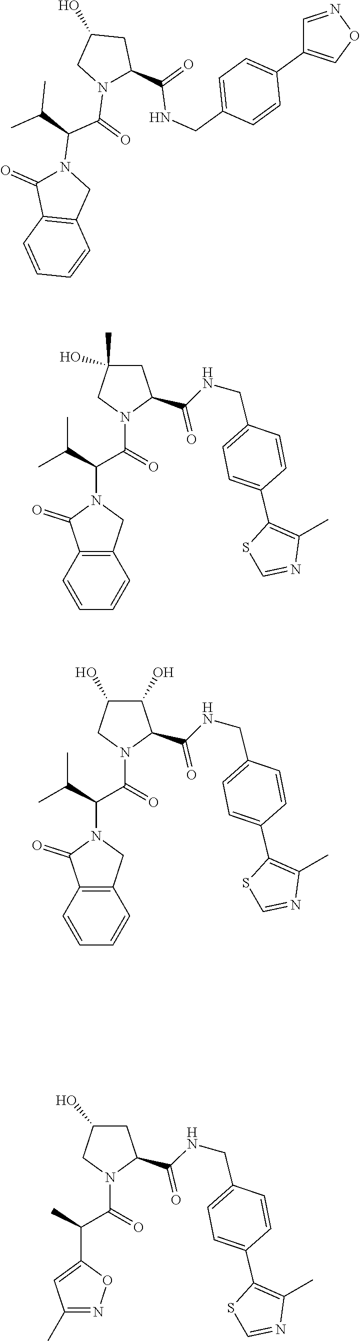

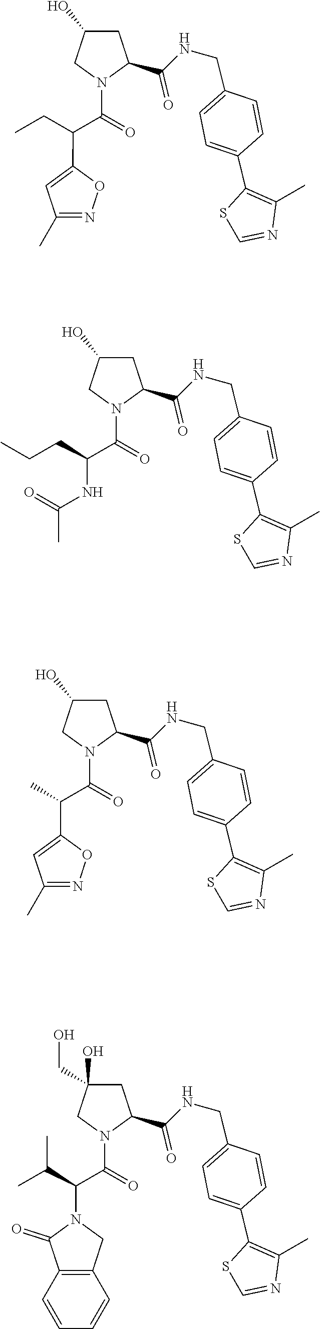

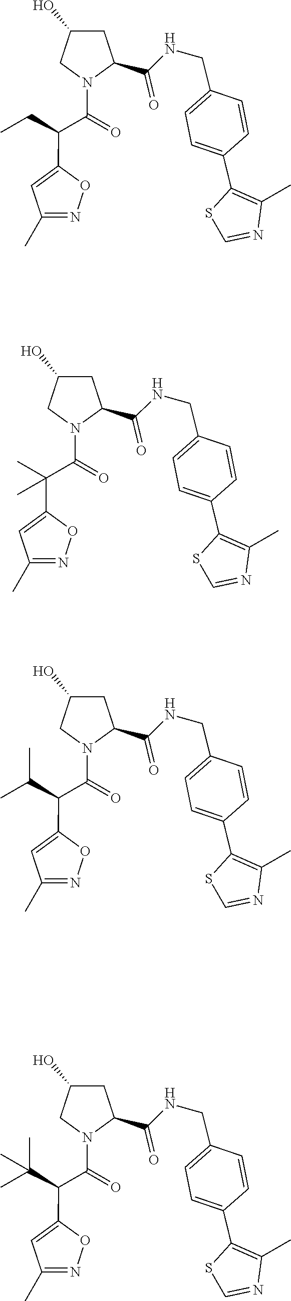

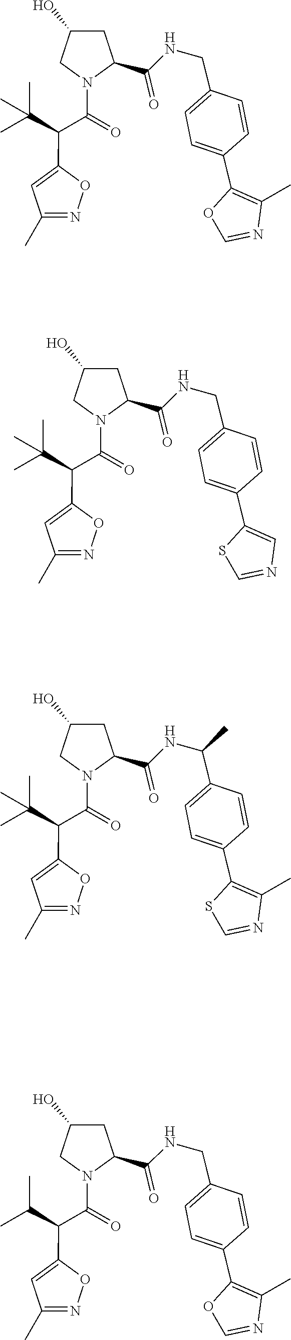

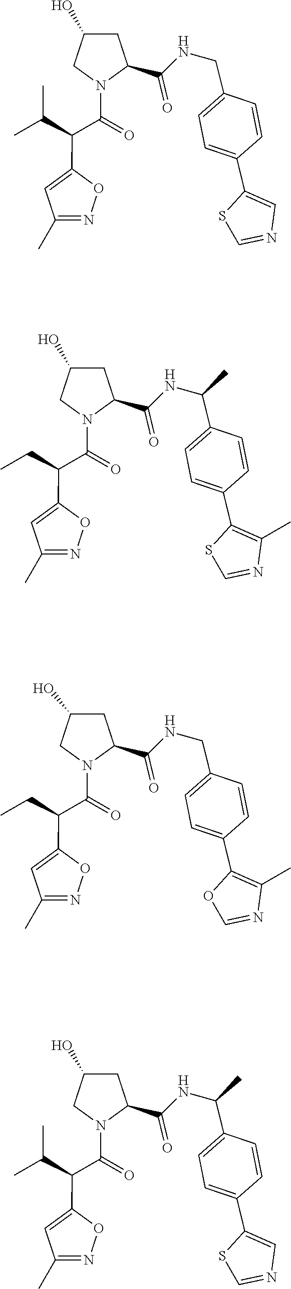

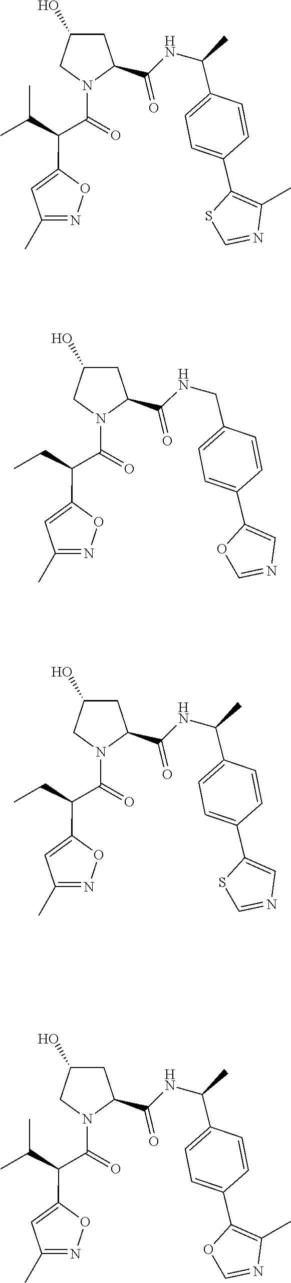

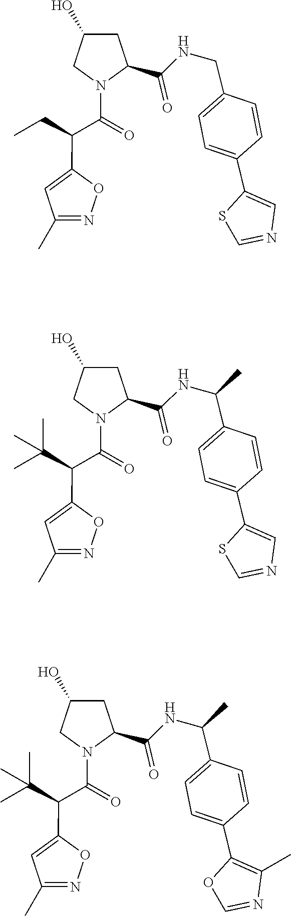 [SMILES: CC[C@@H](C(=O)N1C[C@H](O)C[C@H]1C(=O)NCc1ccc(-c2cncs2)cc1)c1cc(C)no1 ; Cc1cc([C@H](C(=O)N2C[C@H](O)C[C@H]2C(=O)N[C@@H](C)c2ccc(-c3cncs3)cc2)C(C)(C)C)on1 ; Cc1cc([C@H](C(=O)N2C[C@H](O)C[C@H]2C(=O)N[C@@H](C)c2ccc(-c3ocnc3C)cc2)C(C)(C)C)on1]

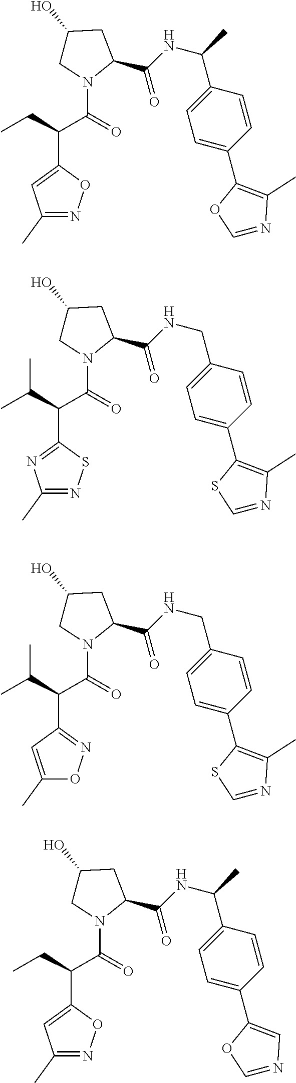

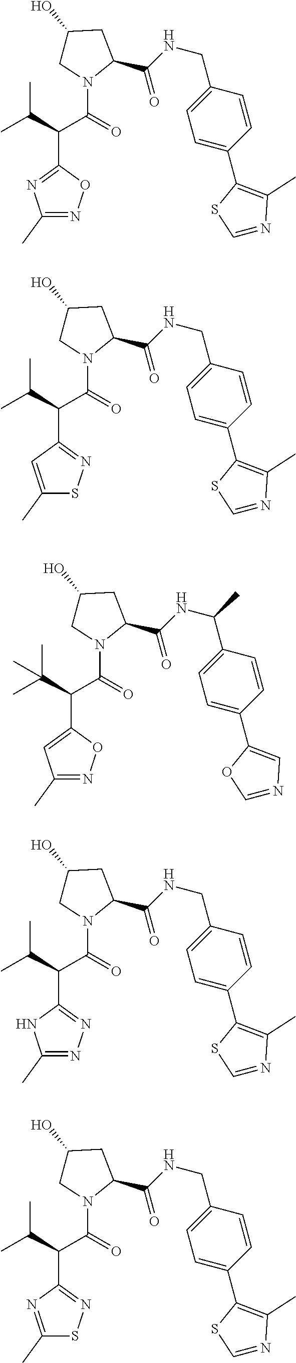

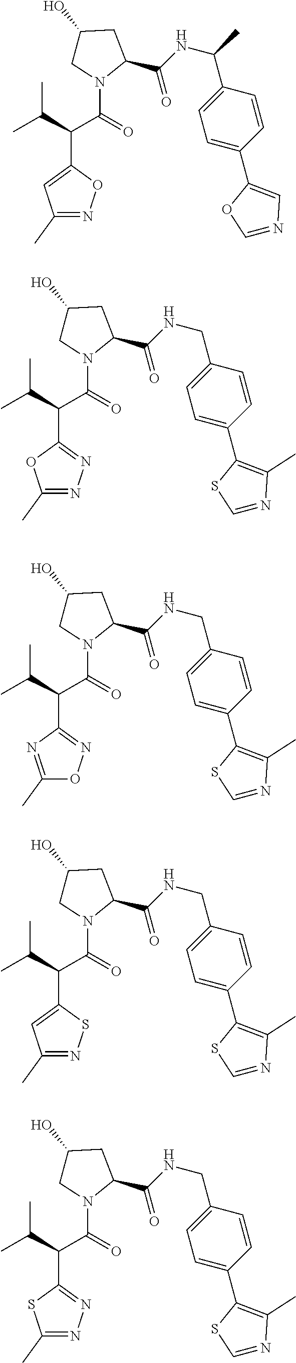

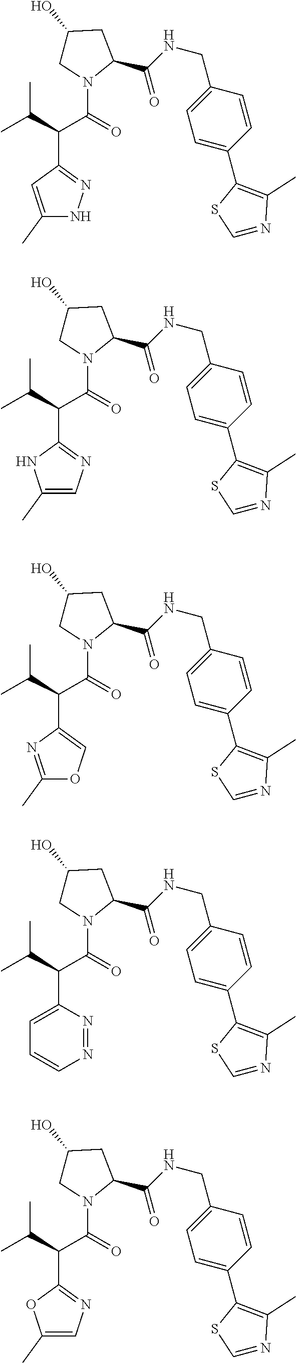

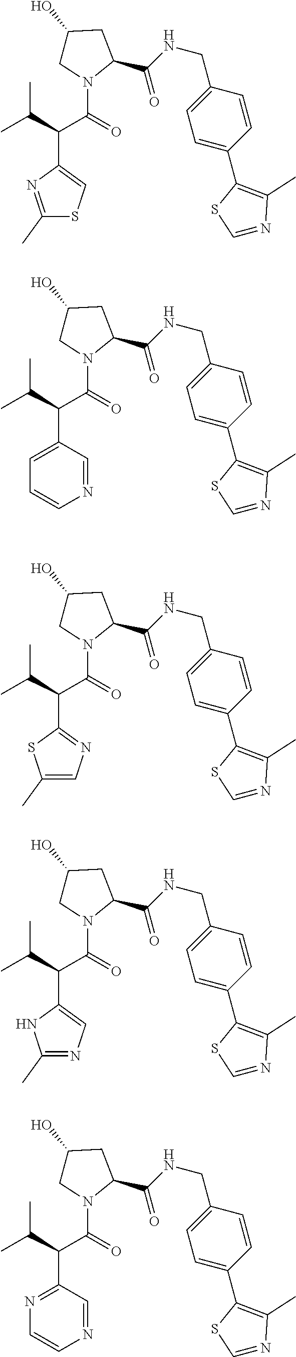

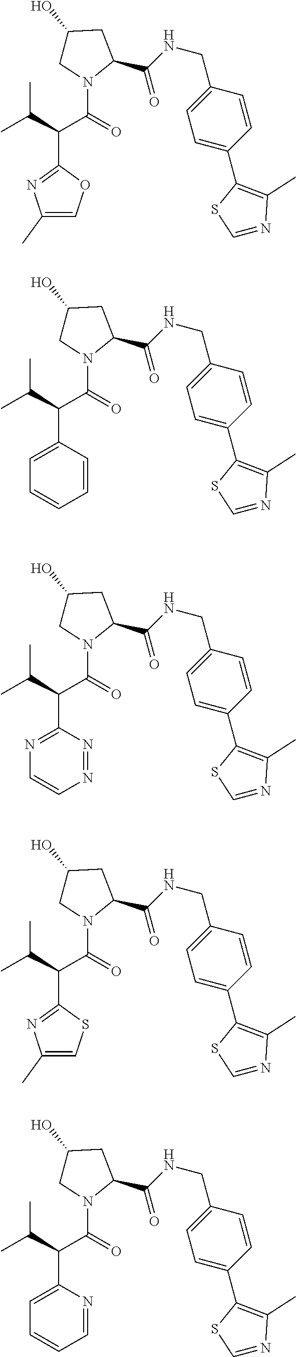

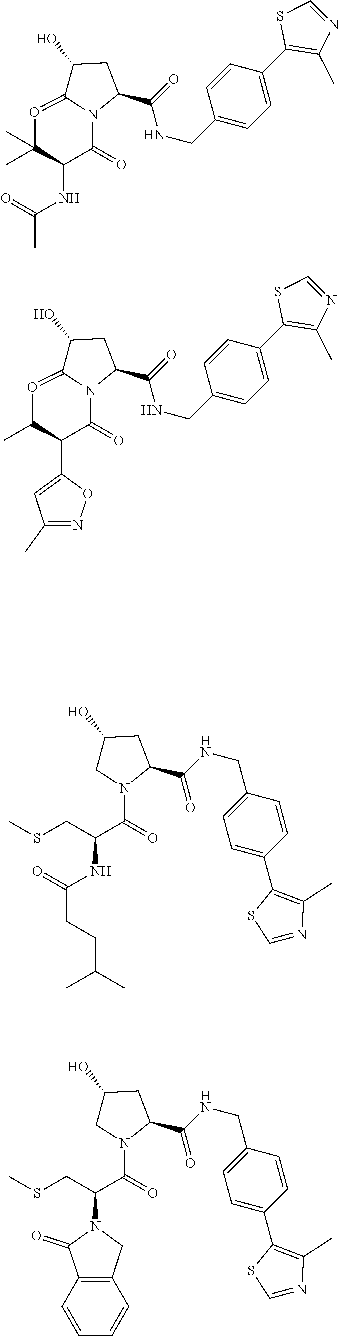

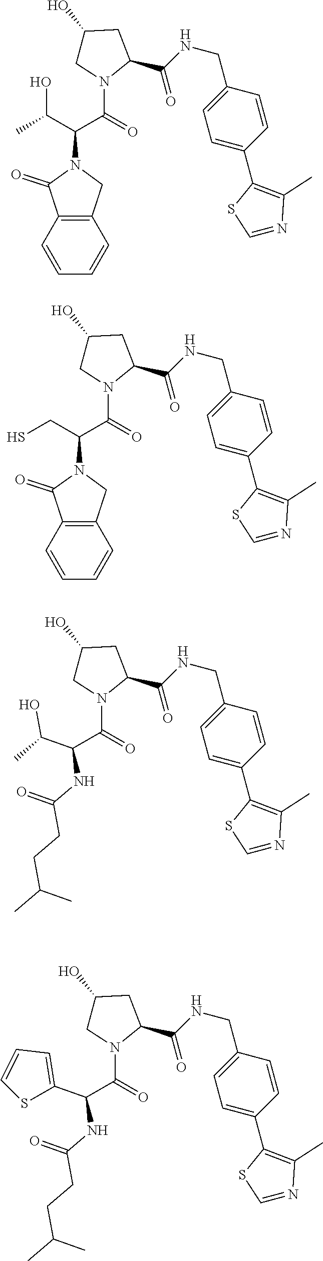

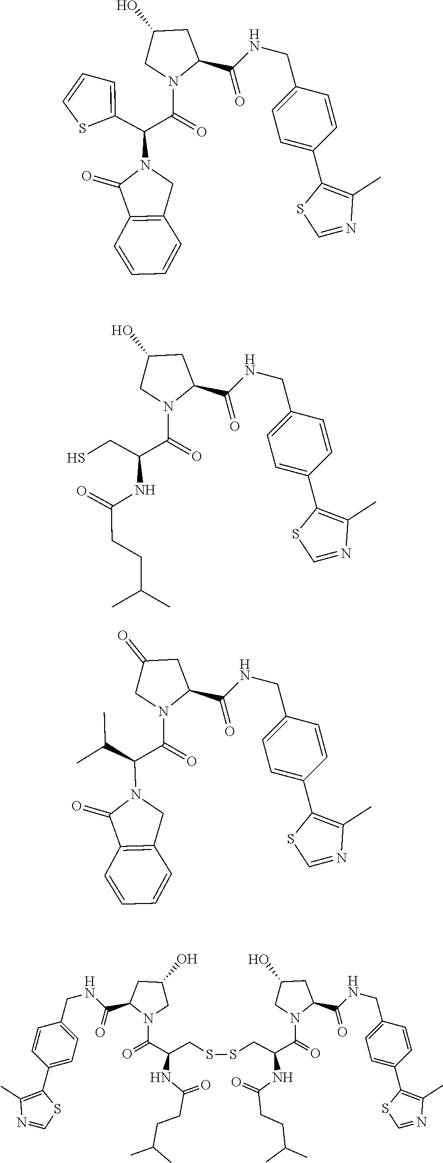

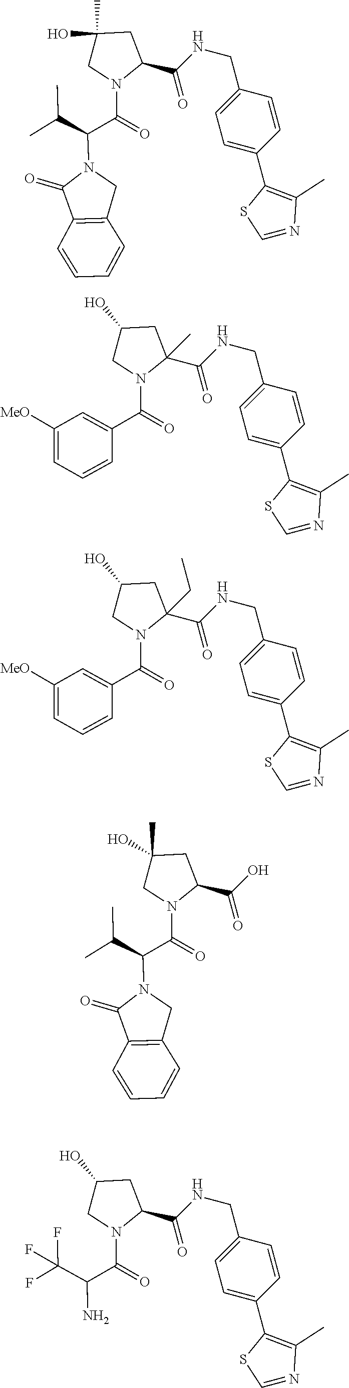

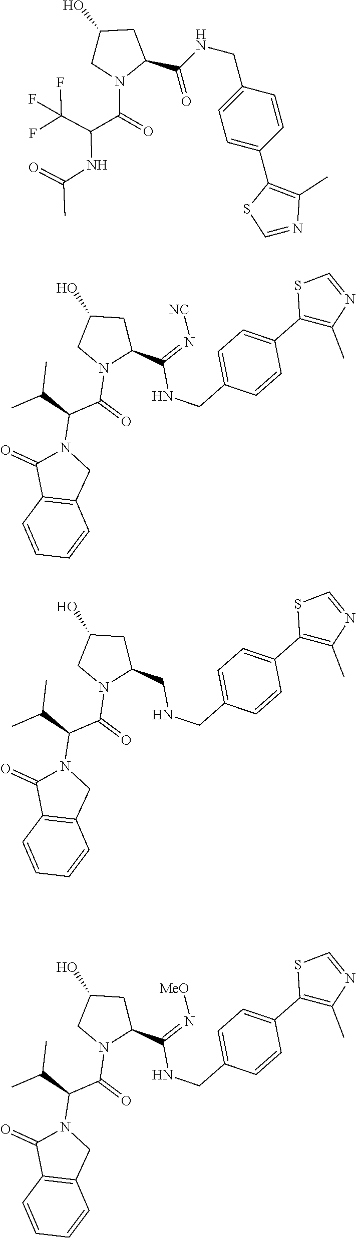 [SMILES: CC(=O)NC(C(=O)N1C[C@H](O)C[C@H]1C(=O)NCc1ccc(-c2scnc2C)cc1)C(F)(F)F ; Cc1ncsc1-c1ccc(CN/C(=N/C#N)[C@@H]2C[C@@H](O)CN2C(=O)[C@H](C(C)C)N2Cc3ccccc3C2=O)cc1 ; Cc1ncsc1-c1ccc(CNC[C@@H]2C[C@@H](O)CN2C(=O)[C@H](C(C)C)N2Cc3ccccc3C2=O)cc1 ; CO/N=C(/NCc1ccc(-c2scnc2C)cc1)[C@@H]1C[C@@H](O)CN1C(=O)[C@H](C(C)C)N1Cc2ccccc2C1=O]

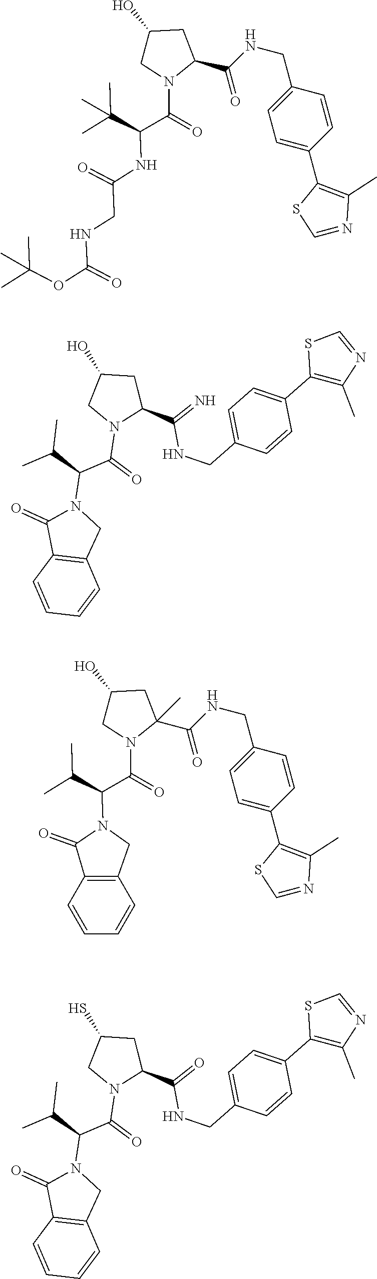 [SMILES: Cc1ncsc1-c1ccc(CNC(=O)[C@@H]2C[C@@H](O)CN2C(=O)[C@@H](NC(=O)CNC(=O)OC(C)(C)C)C(C)(C)C)cc1 ; Cc1ncsc1-c1ccc(CNC(=N)[C@@H]2C[C@@H](O)CN2C(=O)[C@H](C(C)C)N2Cc3ccccc3C2=O)cc1 ; Cc1ncsc1-c1ccc(CNC(=O)C2(C)C[C@@H](O)CN2C(=O)[C@H](C(C)C)N2Cc3ccccc3C2=O)cc1 ; Cc1ncsc1-c1ccc(CNC(=O)[C@@H]2C[C@@H](S)CN2C(=O)[C@H](C(C)C)N2Cc3ccccc3C2=O)cc1]

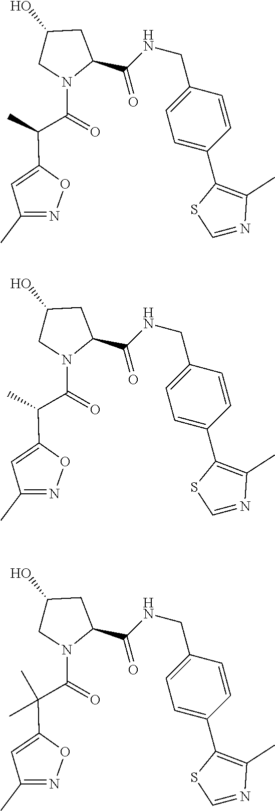

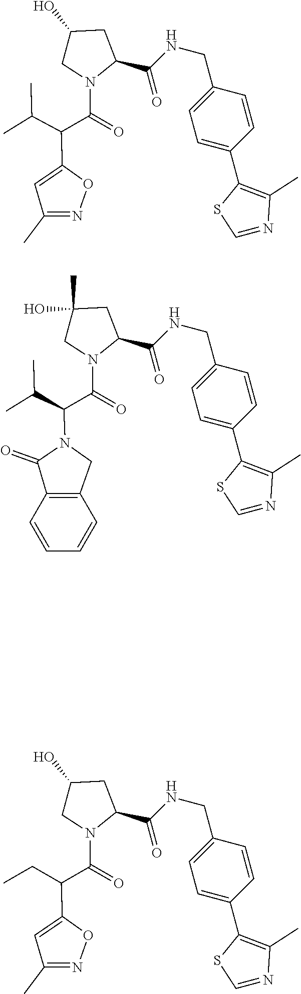

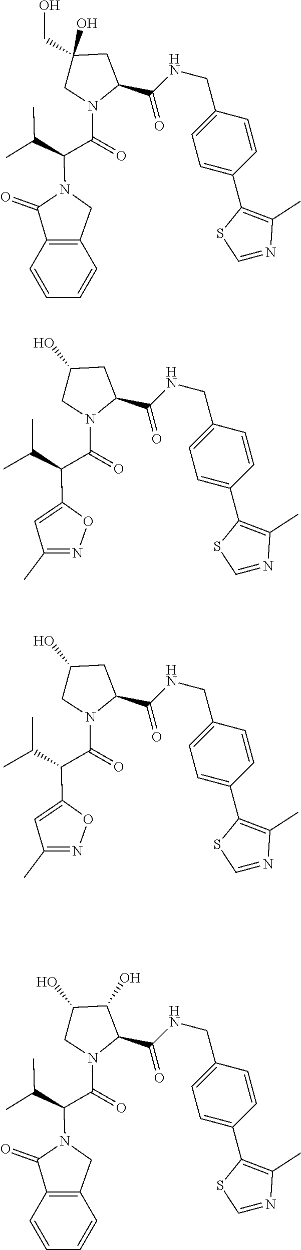

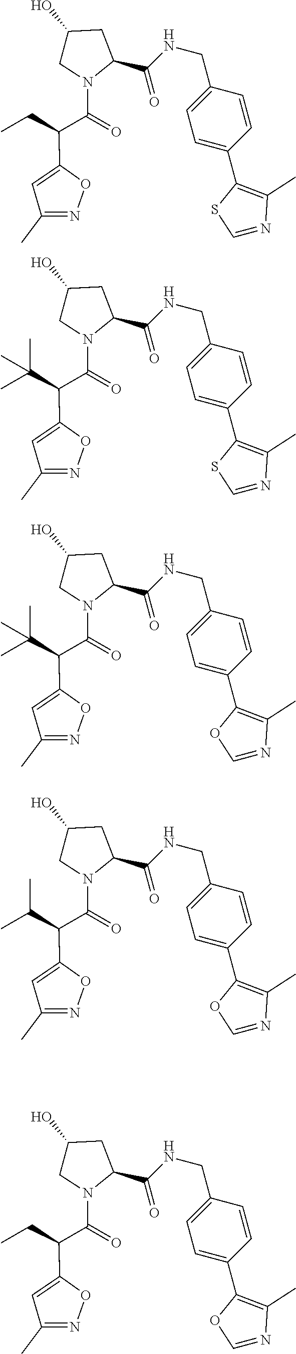

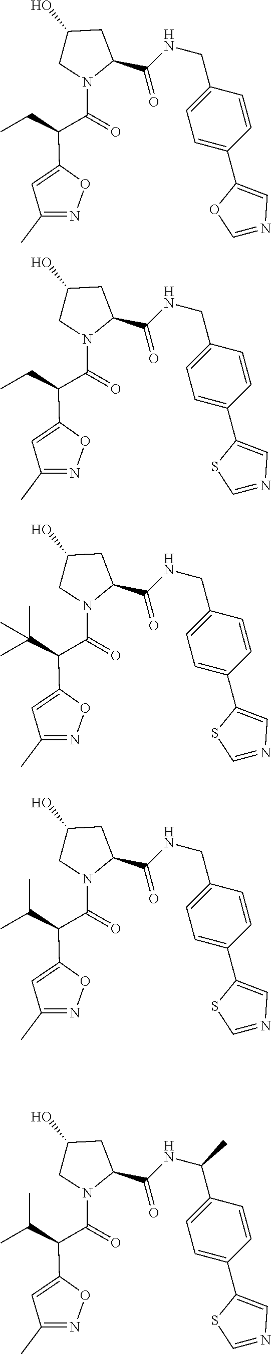

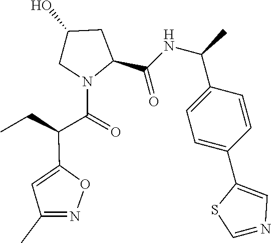 [SMILES: CC[C@@H](C(=O)N1C[C@H](O)C[C@H]1C(=O)N[C@@H](C)c1ccc(-c2cncs2)cc1)c1cc(C)no1]

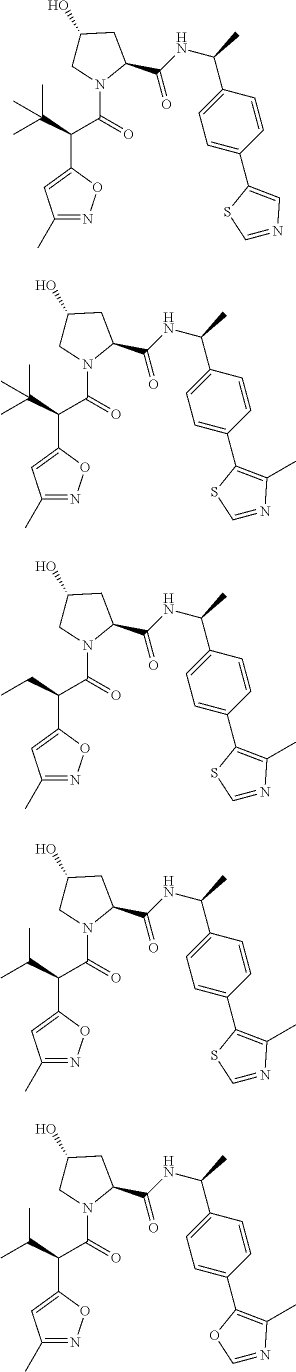

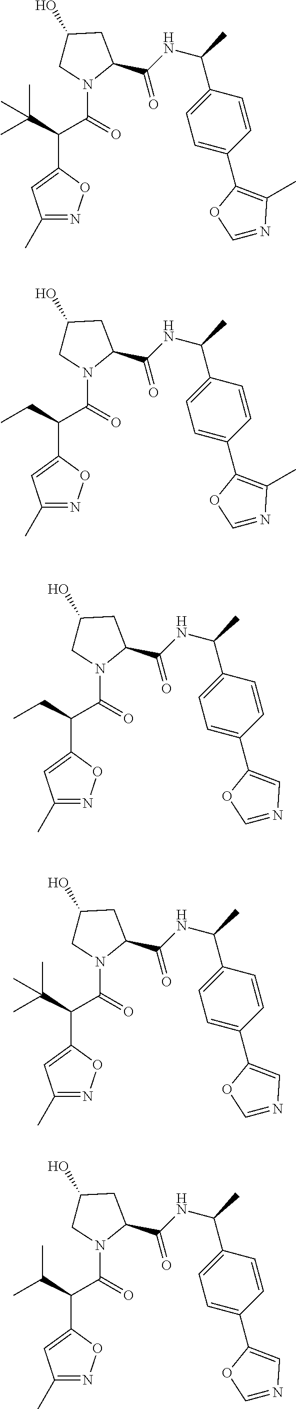

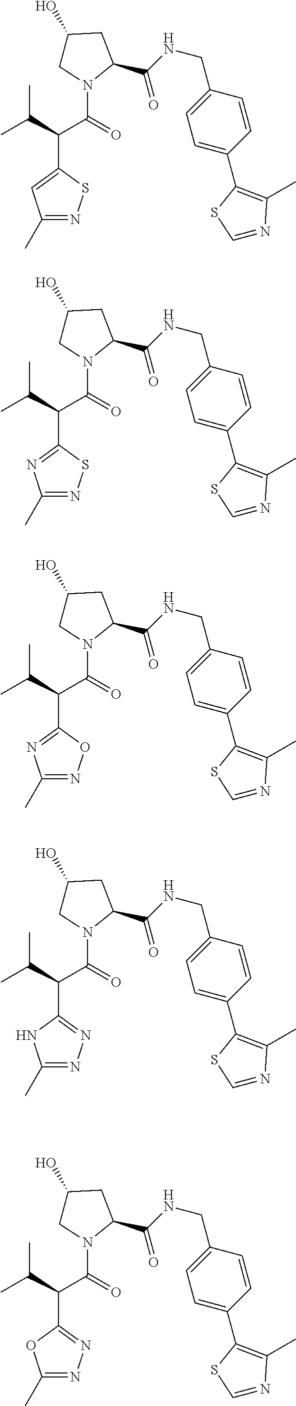

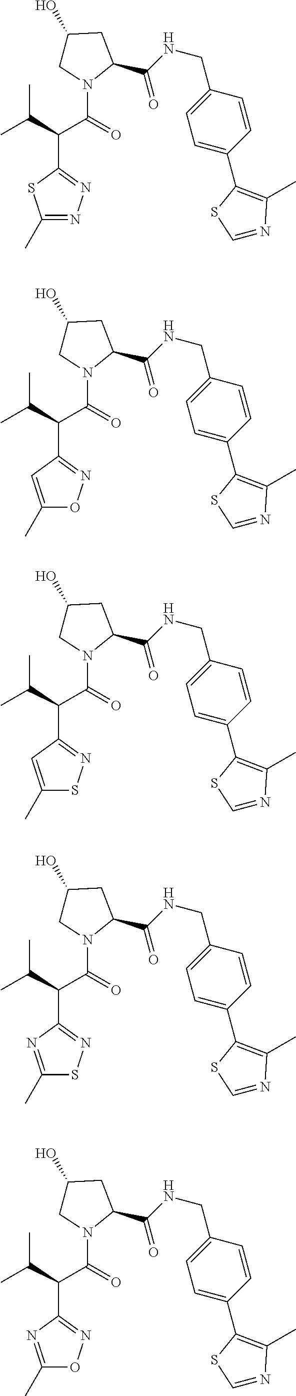

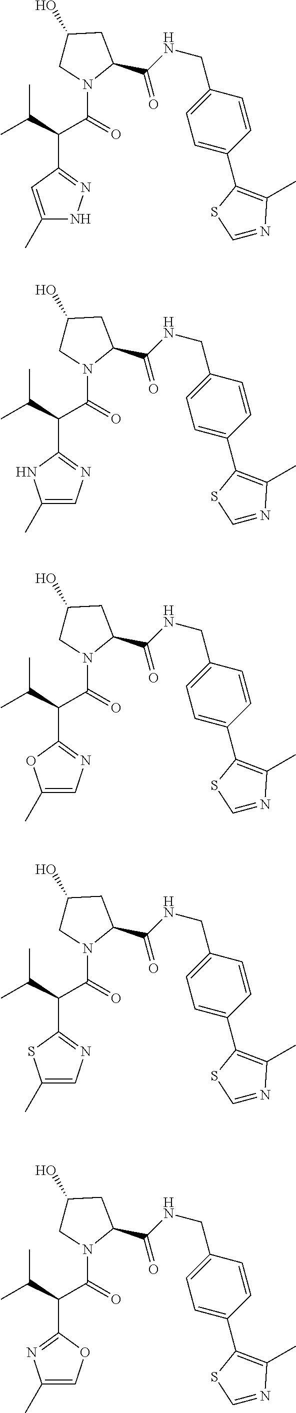

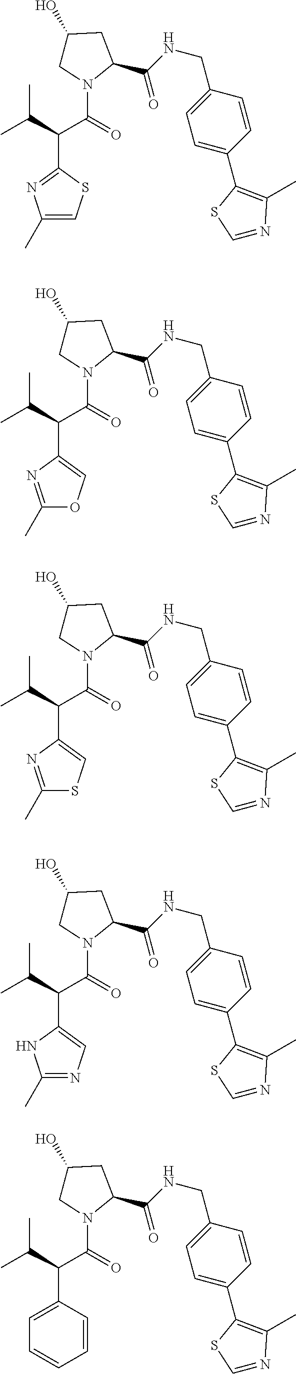

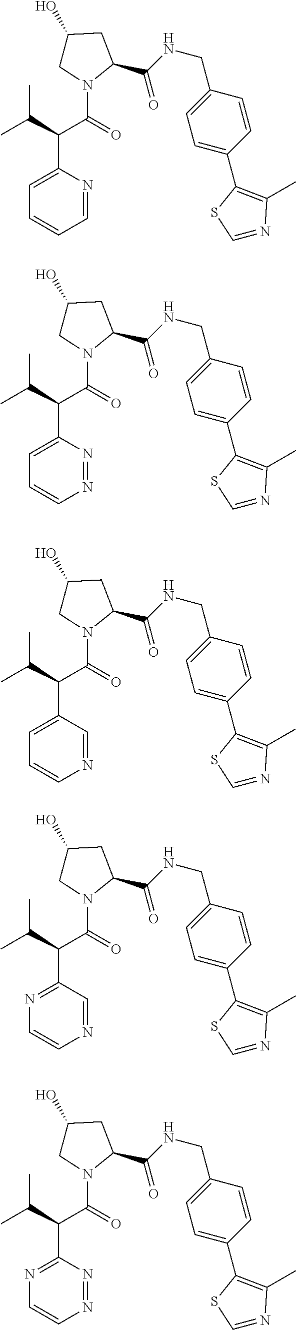

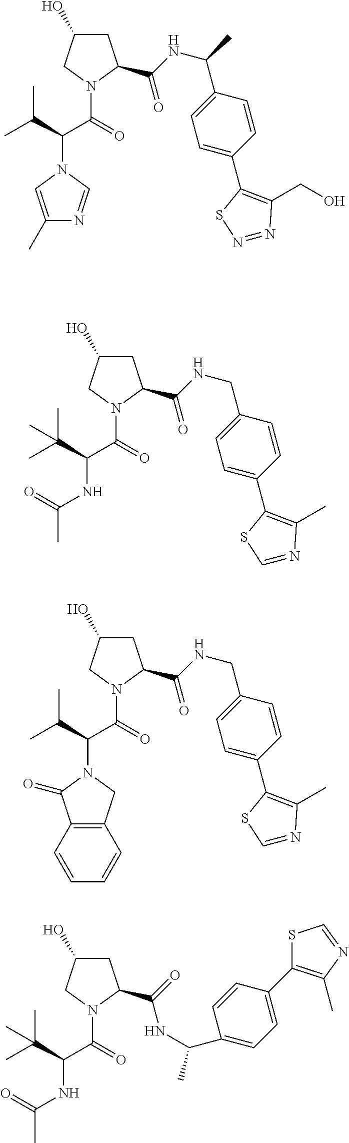

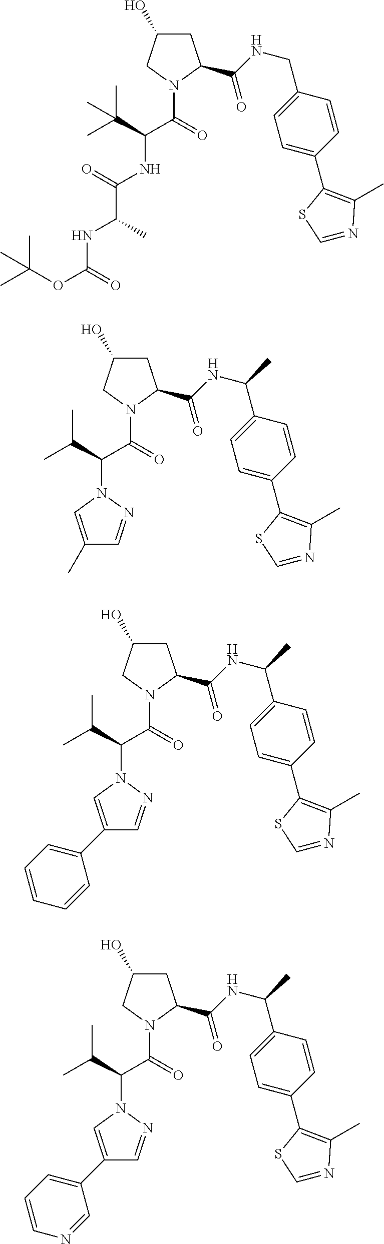

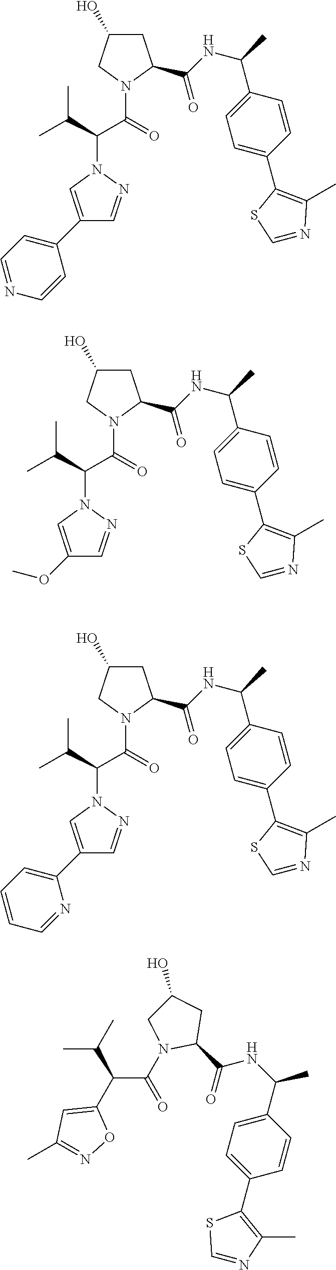

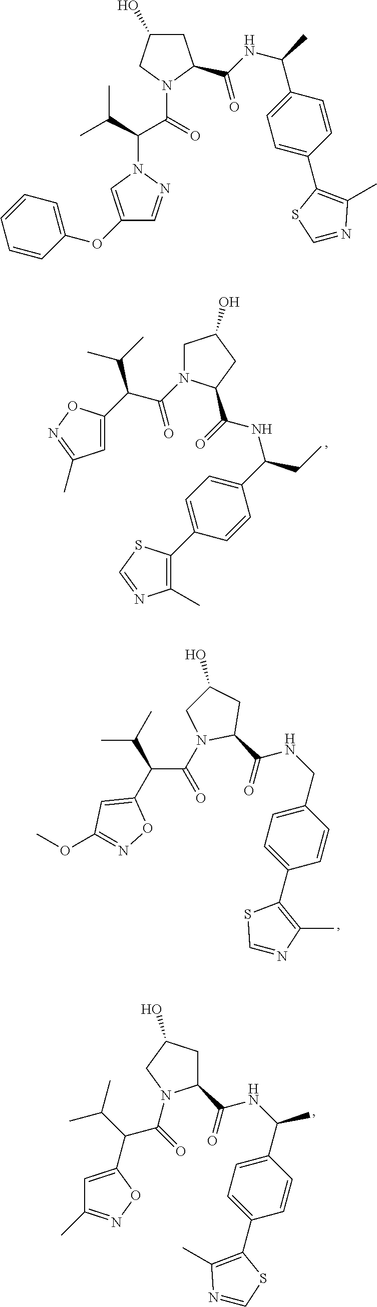

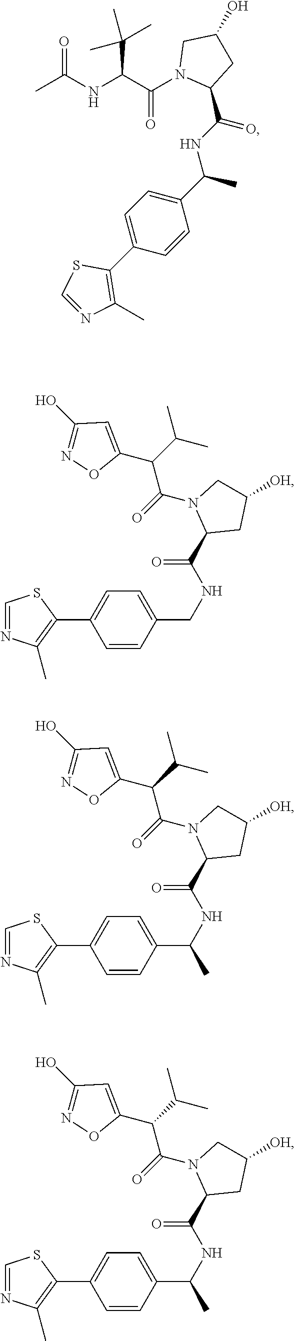 [SMILES: CC(=O)N[C@H](C(=O)N1C[C@H](O)C[C@H]1C(=O)N[C@@H](C)c1ccc(-c2scnc2C)cc1)C(C)(C)C ; Cc1ncsc1-c1ccc(CNC(=O)[C@@H]2C[C@@H](O)CN2C(=O)C(c2cc(O)no2)C(C)C)cc1 ; Cc1ncsc1-c1ccc([C@H](C)NC(=O)[C@@H]2C[C@@H](O)CN2C(=O)[C@@H](c2cc(O)no2)C(C)C)cc1 ; Cc1ncsc1-c1ccc([C@H](C)NC(=O)[C@@H]2C[C@@H](O)CN2C(=O)[C@H](c2cc(O)no2)C(C)C)cc1]

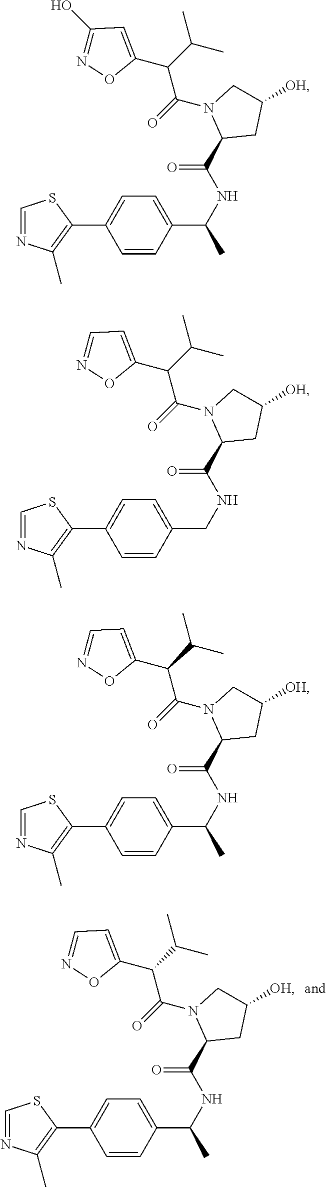 [SMILES: Cc1ncsc1-c1ccc([C@H](C)NC(=O)[C@@H]2C[C@@H](O)CN2C(=O)C(c2cc(O)no2)C(C)C)cc1 ; Cc1ncsc1-c1ccc(CNC(=O)[C@@H]2C[C@@H](O)CN2C(=O)C(c2ccno2)C(C)C)cc1 ; Cc1ncsc1-c1ccc([C@H](C)NC(=O)[C@@H]2C[C@@H](O)CN2C(=O)[C@@H](c2ccno2)C(C)C)cc1 ; Cc1ncsc1-c1ccc([C@H](C)NC(=O)[C@@H]2C[C@@H](O)CN2C(=O)[C@H](c2ccno2)C(C)C)cc1 ; "and"]

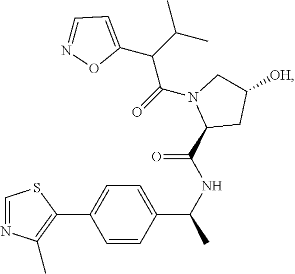 [SMILES: Cc1ncsc1-c1ccc([C@H](C)NC(=O)[C@@H]2C[C@@H](O)CN2C(=O)C(c2ccno2)C(C)C)cc1]

wherein the VLM may be connected to a PTM via a linker, as described herein, at any appropriate location, including, e.g., an aryl, heteroary, phenyl, or phenyl of an indole group, optionally via any appropriate functional group, such as an amine, ester, ether, alkyl, or alkoxy.

In any of the aspects or embodiments described herein, the ULM is a ULM as provided in Table 1.

### Exemplary Linkers

In any of the aspects or embodiments described herein, the compound includes a Linker (L) as described herein.

In certain embodiments, the compounds as described herein include a means for chemically coupling the PTM to the ULM, e.g., one or more PTMs chemically linked or coupled to one or more ULMs (e.g., at least one of VLM) via a chemical linker (L). In certain embodiments, the linker group L is a group comprising one or more covalently connected structural units (e.g., -A^{L}ₗ . . . (A^{L})_{q}- or -(A^{L})_{q}-), wherein A^{L}, is a group coupled to PTM, and (A^{L})_{q} is a group coupled to ULM.

In any aspect or embodiment described herein, the linker (L) to ULM (e.g., VLM,) connection or coupling is a stable L-ULM connection. For example, in any aspect or embodiment described herein, when a linker (L) and a ULM is connected via a heteroatom, any subsequent heteroatom, if present, is separated by at least one single carbon atom (e.g., —CH₂—), such as with an acetal or aminal group. By way of further example, in any aspect or embodiment described herein, when a linker (L) and a ULM is connected via a heteroatom, the heteroatom is not part of a ester.

In any aspect or embodiment described herein, the linker group L is a bond or a chemical linker group represented by the formula -(A^{L})_{q}-, wherein A is a chemical moiety and q is an integer from 1-100 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80), and wherein L is covalently bound to both the PTM and the ULM, and provides for binding of the PTM to the protein target and the ULM to an E3 ubiquitin ligase to effectuate target protein ubiquitination.

In any aspect or embodiment described herein, the linker group L is a bond or a chemical linker group represented by the formula -(A^{L})_{q}-, wherein A is a chemical moiety and q is an integer from 6-30 (e.g., 1, 2, 3, 4, 5, 6, 7, 8, 9, 10, 11, 12, 13, 14, 15, 16, 17, 18, 19, 20, 21, 22, 23, 24, or 25), and wherein L is covalently bound to both the PTM and the ULM, and provides for binding of the PTM to the protein target and the ULM to an E3 ubiquitin ligase in sufficient proximity to result in target protein ubiquitination.

In any aspect or embodiment described herein, the linker group L is -(A^{L})_{q}-, wherein:

(A^{L})_{q} is a group which is connected to at least one of a ULM (such as a VLM), PTM moiety, or a combination thereof;
q of the linker is an integer greater than or equal to 1;
each A^{L} is independently selected from the group consisting of, a bond, CR^{L1}R^{L2}, O, S, SO, SO₂, NR^{L3}, SO₂NR^{L3}, SONR^{L3}, CONR^{L3}, NR^{L3}CONR^{L4}, NR^{L3}SO₂NR^{L4}, CO, CR^{L1}═CR^{L2}, C≡C, SiR^{L1}R^{L2}, P(O)R^{L1}, P(O)OR^{L1}, NR^{L3}C(═NCN)NR^{L4}, NR^{L3}C(═NCN), NR^{L3}C(═CNO₂)NR^{L4}, C₃₋₁₁cycloalkyl optionally substituted with 0-6 R^{L1} and/or R^{L2} groups, C₅₋₁₃ spirocycloalkyl optionally substituted with 0-9 R^{L1} and/or R^{L2} groups, C₃₋₁₁heterocyclyl optionally substituted with 0-6 R^{L1} and/or R^{L2} groups, C₅₋₁₃ spiroheterocyclyl optionally substituted with 0-8 R^{L1} and/or R^{L2} groups, aryl optionally substituted with 0-6 R^{L1} and/or R^{L2} groups, heteroaryl optionally substituted with 0-6 R^{L1} and/or R^{L2} groups, where R^{L1} or R^{L2}, each independently are optionally linked to other groups to form cycloalkyl and/or heterocyclyl moiety, optionally substituted with 0-4 R^{L5} groups; and
R^{L1}, R^{L2}, R^{L3}, R^{L} and R^{L5} are, each independently, H, halo, C₁₋₈alkyl, OC₁₋₈alkyl, SC₁₋₈alkyl, NHC₁₋₈alkyl, N(C₁₋₈alkyl)₂, C₃₋₁₁cycloalkyl, aryl, heteroaryl, C₃₋₁₁heterocyclyl, OC₁₋₈cycloalkyl, SC₁₋₈cycloalkyl, NHC₁₋₈cycloalkyl, N(C₁₋₈cycloalkyl)₂, N(C₁₋₈cycloalkyl)(C₁₋₈alkyl), OH, NH₂, SH, SO₂C₁₋₈alkyl, P(O)(OC₁₋₈alkyl)(C₁₋₈alkyl), P(O)(OC₁₋₈alkyl)₂, CC—C₁₋₈alkyl, CCH, CH═CH(C₁₋₈alkyl), C(C₁₋₈alkyl)=CH(C₁₋₈alkyl), C(C₁₋₈alkyl)=C(C₁₋₈alkyl)₂, Si(OH)₃, Si(C₁₋₈alkyl)₃, Si(OH)(C₁₋₈alkyl)₂, COC₁₋₈alkyl, CO₂H, halogen, CN, CF₃, CHF₂, CH₂F, NO₂, SF₅, SO₂NHC₁₋₈alkyl, SO₂N(C₁₋₈alkyl)₂, SONHC₁₋₈alkyl, SON(C₁₋₈alkyl)₂, CONHC₁₋₈alkyl, CON(C₁₋₈alkyl)₂, N(C₁₋₈alkyl)CONH(C₁₋₈alkyl), N(C₁₋₈alkyl)CON(C₁₋₈alkyl)₂, NHCONH(C₁₋₈alkyl), NHCON(C₁₋₈alkyl)₂, NHCONH₂, N(C₁₋₈alkyl)SO₂NH(C₁₋₈alkyl), N(C₁₋₈alkyl) SO₂N(C₁₋₈alkyl)₂, NH SO₂NH(C₁₋₈alkyl), NH SO₂N(C₁₋₈alkyl)₂, NH SO₂NH₂.

In any aspect or embodiment described herein, each A^{L} is independently selected from the group consisting of CR^{L1}R^{L2}, O, NR^{L3}, CONR^{L3}, CO, CR^{L1}=CR^{L2}, C≡C, C₃₋₁₁cycloalkyl optionally substituted with 1-6 R^{L1} and/or R^{L2} groups, C₃₋₁₁heteocyclyl optionally substituted with 1-6 R^{L1} and/or R^{L2} groups, aryl optionally substituted with 1-6 R^{L1} and/or R^{L2} groups, and heteroaryl optionally substituted with 1-6 R^{L1} and/or R^{L2} groups, where R^{L1} or R^{L2}, each independently are optionally linked to other groups to form cycloalkyl and/or heterocyclyl moiety, optionally substituted with 1-4 R^{L5} groups, and R^{L1}, R^{L2}, R^{L3}, and R^{L5} are, each independently, halogen, C₁₋₈alkyl, OC₁₋₈alkyl, NHC₁₋₈alkyl, N(C₁₋₈alkyl)₂, C₃₋₁₁cycloalkyl, aryl, heteroaryl, C₃₋₁₁heterocyclyl, OC₃₋₈cycloalkyl, NHC₃₋₈cycloalkyl, N(C₃₋₈cycloalkyl)(C₁₋₈alkyl), OH, NH₂, CCH, COC₁₋₈alkyl, CO₂H, CN, CF₃, CHF₂, CH₂F, or NO₂.

In certain embodiments, q of the linker is an integer greater than or equal to 0. In certain embodiments, q is an integer greater than or equal to 1.

In certain embodiments, e.g., where q of the linker is greater than 2, (A^{L})_{q} is a group which is to A^{L}ₗ and (A^{L})_{q} wherein the units A^{L} couple a PTM to a ULM.

In certain embodiments, e.g., where q of the linker is 2, (A^{L})_{q} is a group which is connected to A^{L}ₗ and to a ULM or PTM.

In certain embodiments, e.g., where q of the linker is 1, the structure of the linker group L is -A^{L1}-, and A^{L}ₗ is a group which is connected to a ULM moiety and a PTM moiety.

In certain embodiments, the unit A^{L} of linker (L) comprises a group represented by a general structure selected from the group consisting of:
—NR(CH₂)ₙ-(lower alkyl)-, —NR(CH₂)ₙ-(lower alkoxyl)-, —NR(CH₂)ₙ-(lower alkoxyl)-OCH₂—, —NR(CH₂)ₙ-(lower alkoxyl)-(lower alkyl)-OCH₂—, —NR(CH₂)ₙ-(cycloalkyl)-(lower alkyl)-OCH₂—, —NR(CH₂)ₙ-(hetero cycloalkyl)-, —NR(CH₂CH₂O)ₙ-(lower alkyl)-O—CH₂—, —NR(CH₂CH₂O)ₙ-(hetero cycloalkyl)-O—CH₂—, —NR(CH₂CH₂O)ₙ-Aryl-O—CH₂—, —NR(CH₂CH₂O)ₙ-(hetero aryl)-O—CH₂—, —NR(CH₂CH₂O)ₙ-(cyclo alkyl)-O-(hetero aryl)-O—CH₂—, —NR(CH₂CH₂O)ₙ-(cyclo alkyl)-O-Aryl-O—CH₂—, —NR(CH₂CH₂O)ₙ-(lower alkyl)-NH-Aryl-O—CH₂—, —NR(CH₂CH₂O)ₙ-(lower alkyl)-O-Aryl-CH₂, —NR(CH₂CH₂O)ₙ-cycloalkyl-O-Aryl-, —NR(CH₂CH₂O)ₙ-cycloalkyl-O-(heteroaryl)l-, —NR(CH₂CH₂)ₙ-(cycloalkyl)-O-(heterocyclyl)-CH₂, —NR(CH₂CH₂)ₙ— (heterocyclyl)-(heterocyclyl)-CH₂, —N(R₁R₂)-(heterocyclyl)-CH₂; where
n of the linker can be 0 to 10;
R of the linker can be H, lower alkyl;
R¹ and R² of the linker can form a ring with the connecting N.

In any aspect or embodiment described herein, the L is selected from the group consisting of:

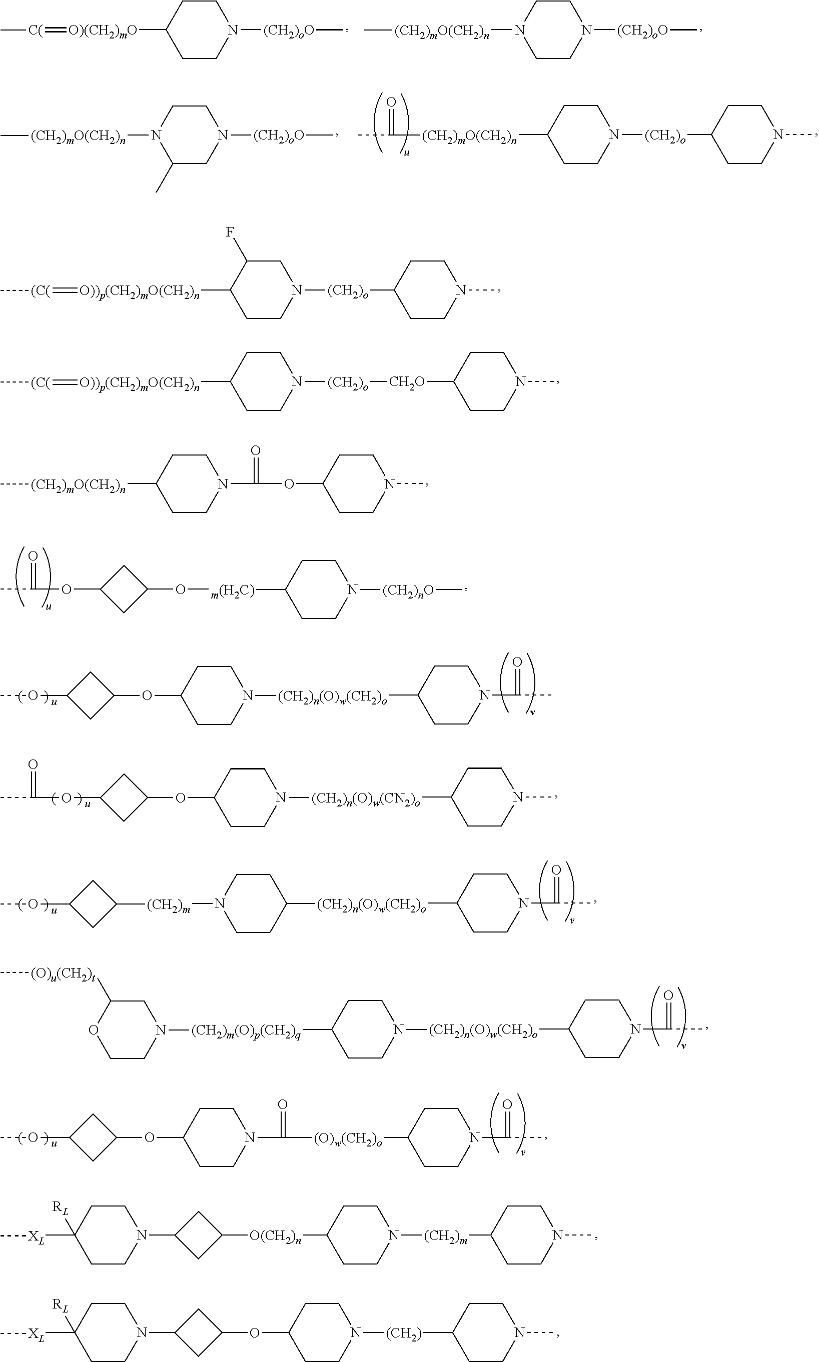

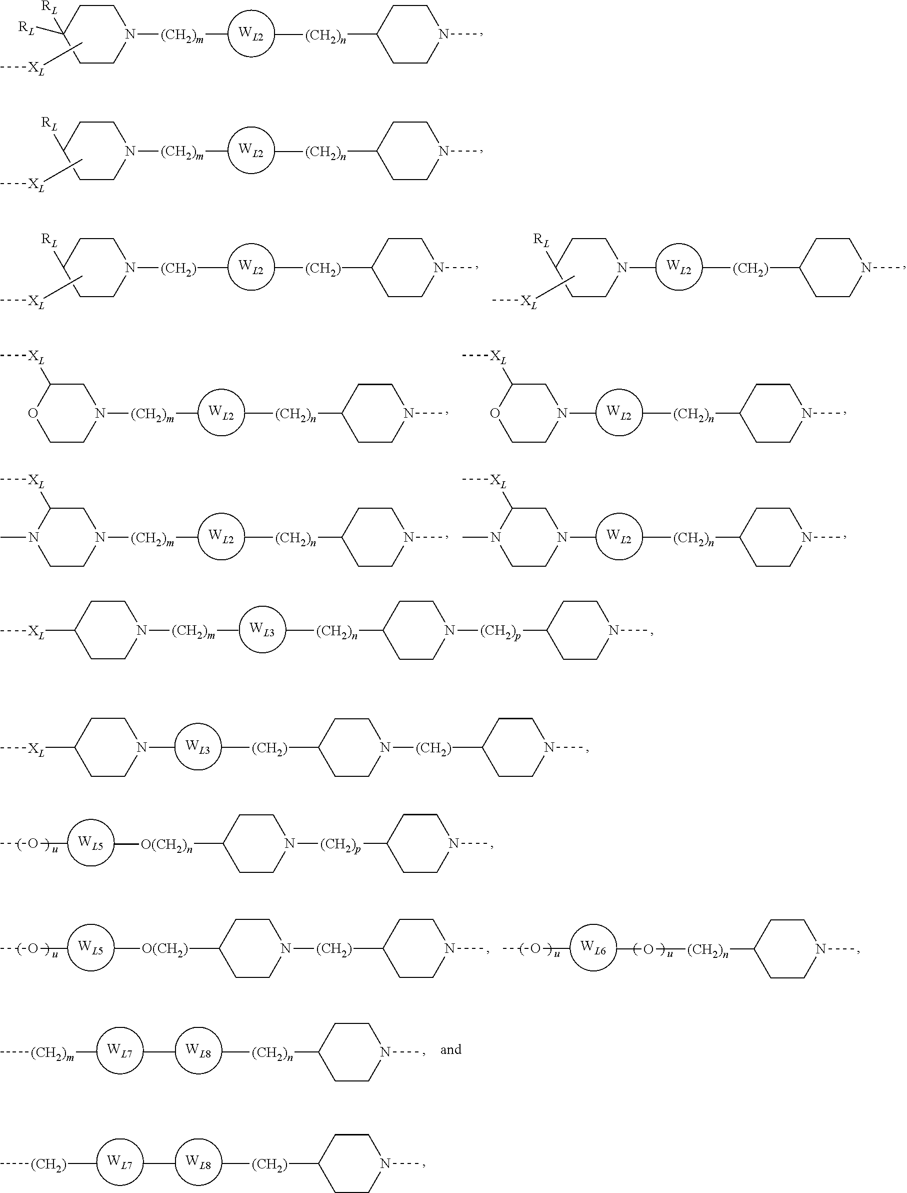

wherein:
each of m, n, o, p, q, and t is independently selected from the integers 0, 1, 2, 3 and 4 (preferably 0, 1, or 2); and
u, w, and v are each independently selected from integers 0 and 1.
X_{L} is —C(CH₂)—, —C(CH₃)H,— —CH₂—, —O—, C═O, or —NH—CH₂—;
R_{L} is H, OH, F, Cl, or methyl;
W_{L2} is selected from an optionally substituted 6-12 membered spirocycloalkylene or spirohetercyclylene (e.g. a 6-12 or 8-12 member spirocycloalkylene or spirohetercyclylene substituted with 0, 1, or 2 substituents selected from hydroxy, halogen, C₁₋₃ alkoxy, C₁₋₃ alkyl, C₁₋₃ haloalkyl, or amino),

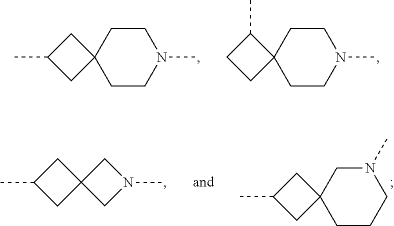

W_{L3} is selected from an optionally substituted 6-12 membered spirocycloalkylene or spirohetercyclylene (e.g. a 6-12 or 8-12 member spirocycloalkylene or spirohetercyclylene substituted with 0, 1, or 2 substituents selected from hydroxy, halogen, C₁₋₃ alkoxy, C₁₋₃ alkyl, C₁₋₃ haloalkyl, or amino), and

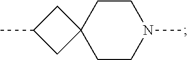

W_{L5} is selected from an optionally substituted 6-12 membered spirocycloalkylene or spirohetercyclylene (e.g. a 6-12 or 8-12 member spirocycloalkylene or spirohetercyclylene substituted with 0, 1, or 2 substituents selected from hydroxy, halogen, C₁₋₃ alkoxy, C₁₋₃ alkyl, C₁₋₃ haloalkyl, or amino),

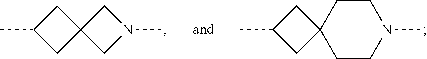

W_{L6} is selected from an optionally substituted 6-12 membered spirocycloalkylene or spirohetercyclylene (e.g. a 6-12 or 8-12 member spirocycloalkylene or spirohetercyclylene substituted with 0, 1, or 2 substituents selected from hydroxy, halogen, C₁₋₃ alkoxy, C₁₋₃ alkyl, C₁₋₃ haloalkyl, or amino),

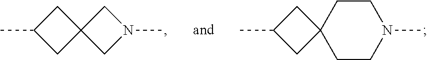

W_{L7} is selected from an optionally substituted 6-12 membered spirocycloalkylene or spirohetercyclylene (e.g. a 6-12 or 8-12 member spirocycloalkylene or spirohetercyclylene substituted with 0, 1, or 2 substituents selected from hydroxy, halogen, C₁₋₃ alkoxy, C₁₋₃ alkyl, C₁₋₃ haloalkyl, or amino),

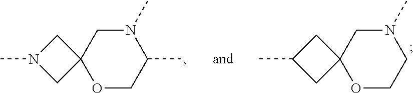

and
W_{L8} is selected from an optionally substituted 6-12 membered spirocycloalkylene or spirohetercyclylene (e.g. a 6-12 or 8-12 member spirocycloalkylene or spirohetercyclylene substituted with 0, 1, or 2 substituents selected from hydroxy, halogen, C₁₋₃ alkoxy, C₁₋₃ alkyl, C₁₋₃ haloalkyl, or amino),

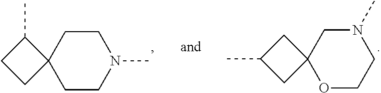

In any aspect or embodiment described herein, W_{L2} is selected from

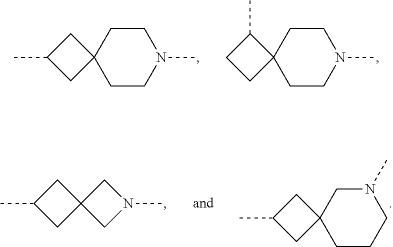

In any aspect or embodiment described herein, W_{L3} is

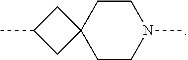

In any aspect or embodiment described herein,

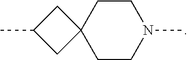

In any aspect or embodiment described herein, W_{L5} is selected from

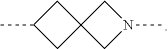

In any aspect or embodiment described herein, W_{L6} is selected from

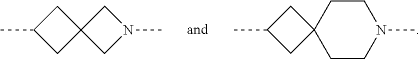

In any aspect or embodiment described herein, W_{L7} is selected from

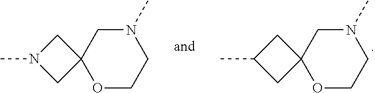

In any aspect or embodiment described herein, W_{L8} is selected from

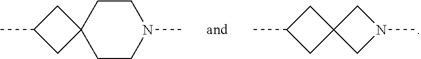

In any aspect or embodiment described herein, W_{L7} is selected from

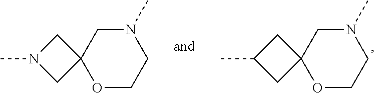

and/or W_{L8} is selected from

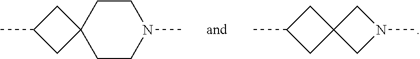

In any aspect or embodiment described herein, each m, n, o, p, q, and t of the chemical linking moiety (L) is independently selected from the integers 0, 1, or 2.

In any aspect or embodiment described herein, the L is selected from the group consisting of:

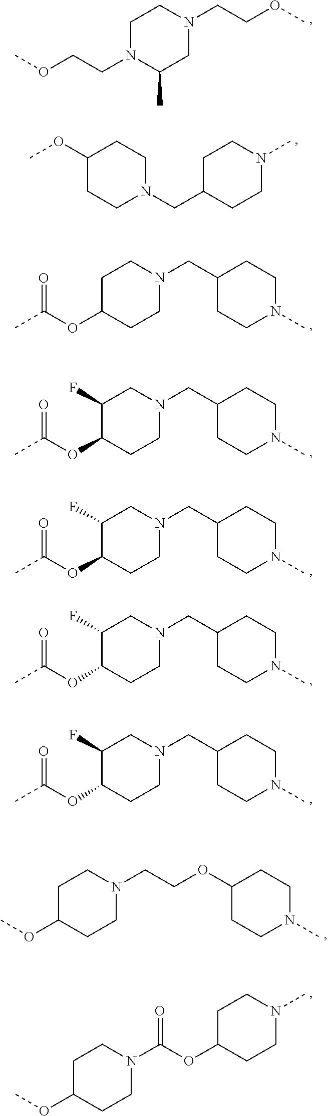

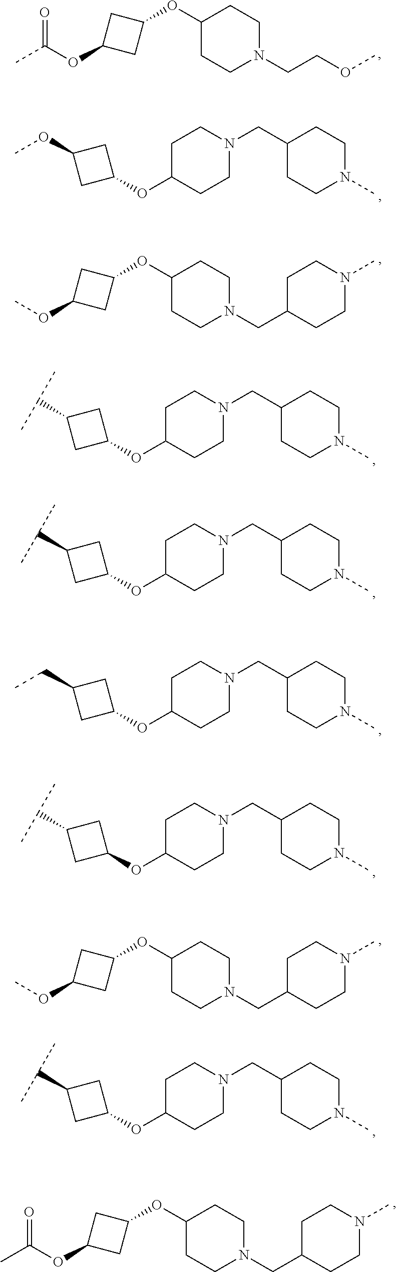

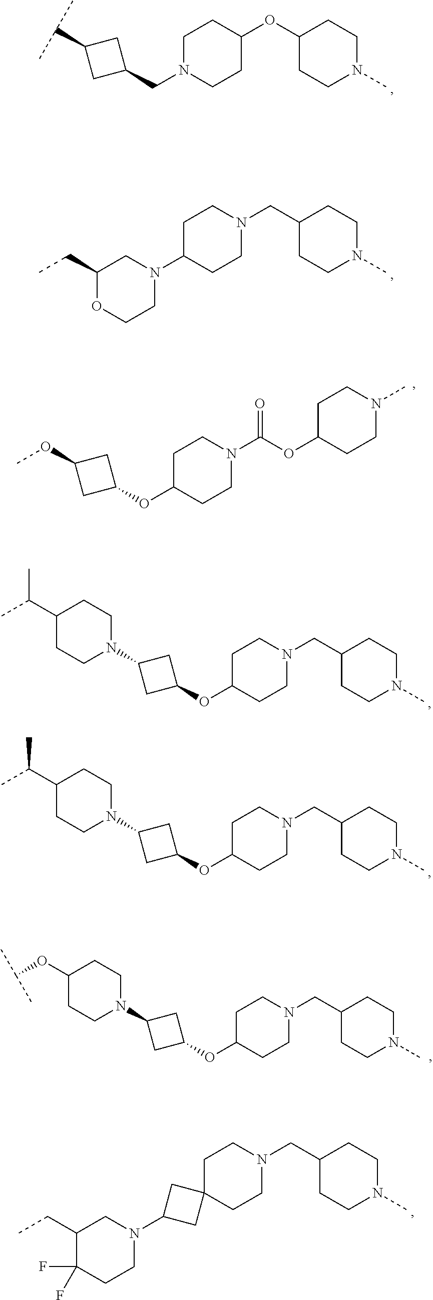

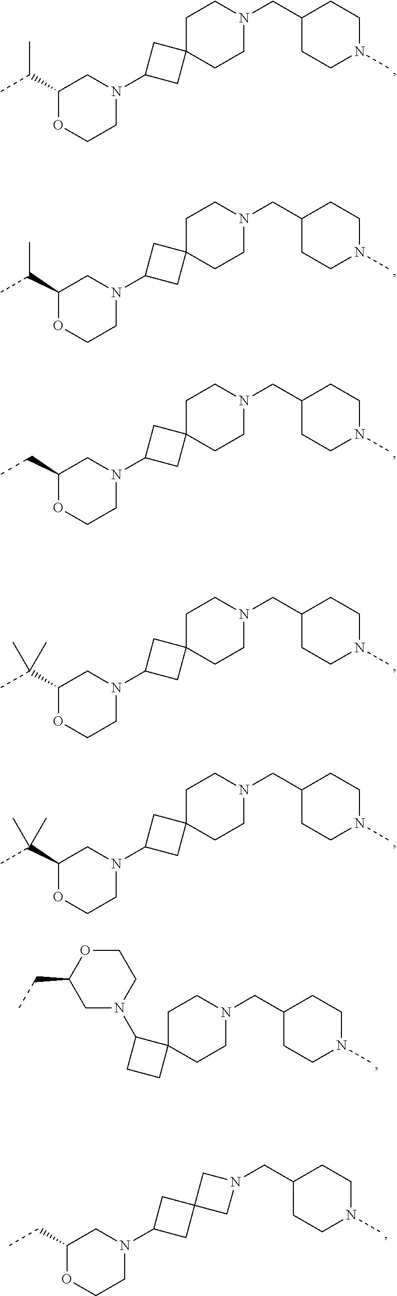

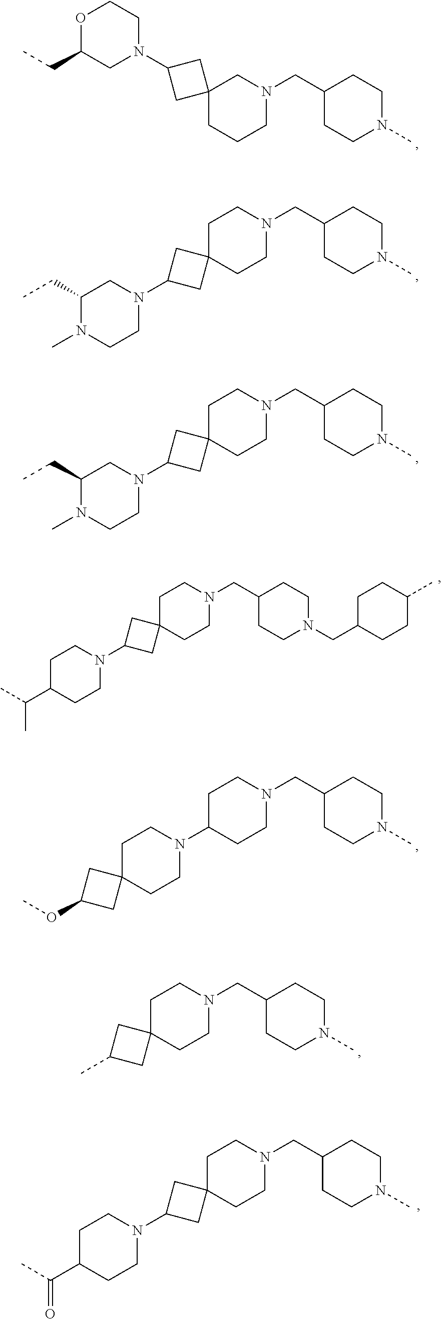

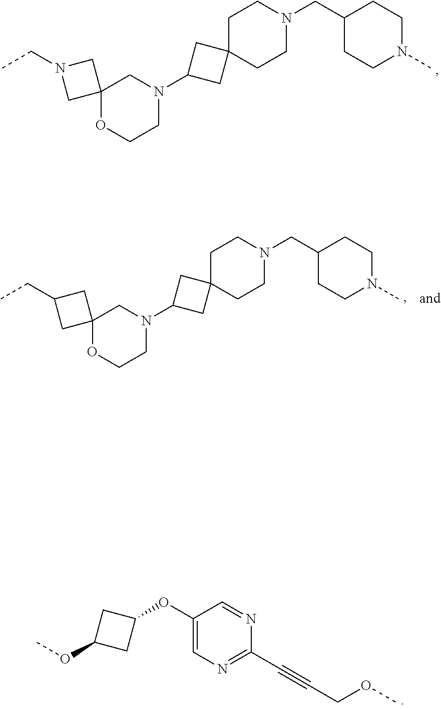

In additional embodiments, the linker (L) comprises a structure selected from, but not limited to the structure shown below, where a dashed line indicates the attachment point to the PTM or ULM moieties:

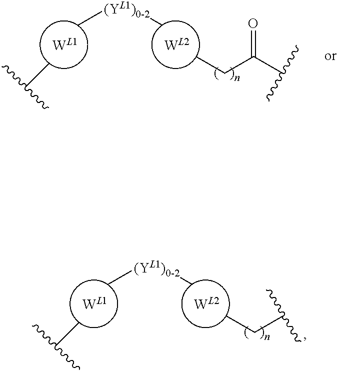

wherein:
W^{L1} and W^{L2} are each independently absent, a 4-8 membered ring with 0-4 heteroatoms, optionally substituted with R^{Q}, each R^{Q} is independently a H, halogen, OH, CN, CF₃, optionally substituted linear or branched C₁₋₆ alkyl, optionally substituted linear or branched C₁₋₆ alkoxy, or two R^{Q} groups taken together with the atom to which they are attached form a 4-8 membered ring system containing 0-4 heteroatoms;
Y^{L1} is each independently a bond, C₁₋₆ alkyl (linear, branched, optionally substituted) and optionally one or more C atoms are replaced with 0; or C₁₋₆ alkoxy (linear, branched, optionally substituted);
n is an integer from 0 to 10; and

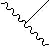

indicates the attachment point to the PTM or the ULM.

In additional embodiments, the linker (L) comprises a structure selected from, but not limited to the structure shown below, where a dashed line indicates the attachment point to the PTM or ULM moieties:

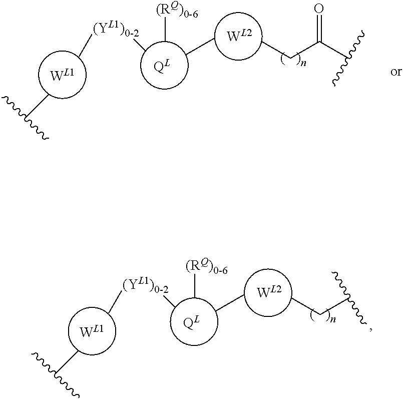

wherein:
W^{L1} and W^{L2} are each independently absent, aryl, heteroaryl, cyclic, heterocyclyl, C₁₋₆ alkyl and optionally one or more C atoms are replaced with O or N, C₁₋₆ alkenyl and optionally one or more C atoms are replaced with O, C₁₋₆ alkynyl and optionally one or more C atoms are replaced with O, bicyclic, biaryl, biheteroaryl, or biheterocyclyl, each optionally substituted with R^{Q}, each R^{Q} is independently a H, halo, OH, CN, CF₃, hydroxyl, nitro, C≡CH, C₂₋₆ alkenyl, C₂₋₆ alkynyl, optionally substituted linear or branched C₁₋₆ alkyl, optionally substituted linear or branched C₁-C₆ alkoxy, optionally substituted OC₁₋₃alkyl (e.g., optionally substituted by 1 or more —F), OH, NH₂, NR^{Y1}R^{Y2}, CN, or two R^{Q} groups taken together with the atom to which they are attached form a 4-8 membered ring system containing 0-4 heteroatoms;
Y^{L1} is each independently a bond, NR^{YL1}, O, S, NR^{YL2}, CR^{YL1}R^{YL2}, C═O, C═S, SO, SO₂, C₁-C₆ alkyl (linear, branched, optionally substituted) and optionally one or more C atoms are replaced with O; C₁-C₆ alkoxy (linear, branched, optionally substituted);
Q^{L} is a 3-6 membered alicyclic or aromatic ring with 0-4 heteroatoms, optionally bridged, optionally substituted with 0-6 R^{Q}, each R^{Q} is independently H, linear or branched C₁₋₆ alkyl optionally substituted by 1 or more halo or C₁₋₆ alkoxyl, or 2 R^{Q} groups taken together with the atom to which they are attached form a 3-8 membered ring system containing 0-2 heteroatoms;
R^{YL1}, R^{YL2} are each independently H, OH, C₁₋₆ alkyl (linear, branched, optionally substituted by 1 or more halo, C₁₋₆ alkoxyl), or R¹, R² together with the atom to which they are attached form a 3-8 membered ring system containing 0-2 heteroatoms);
n is an integer from 0 to 10; and

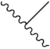

indicates the attachment point to the PTM or the ULM.

In additional embodiments, the linker group is optionally substituted (poly)ethyleneglycol having between 1 and about 100 ethylene glycol units, between about 1 and about 50 ethylene glycol units, between 1 and about 25 ethylene glycol units, between about 1 and 10 ethylene glycol units, between 1 and about 8 ethylene glycol units and 1 and 6 ethylene glycol units, between 2 and 4 ethylene glycol units, or optionally substituted alkyl groups interdispersed with optionally substituted, O, N, S, P or Si atoms. In certain embodiments, the linker is substituted with an aryl, phenyl, benzyl, alkyl, alkylene, or heterocyclyl group. In certain embodiments, the linker may be asymmetric or symmetrical.

In any of the embodiments of the compounds described herein, the linker group may be any suitable moiety as described herein. In one embodiment, the linker is a substituted or unsubstituted polyethylene glycol group ranging in size from about 1 to about 12 ethylene glycol units, between 1 and about 10 ethylene glycol units, about 2 about 6 ethylene glycol units, between about 2 and 5 ethylene glycol units, between about 2 and 4 ethylene glycol units.

In another embodiment, the present disclosure is directed to a compound which comprises a PTM group as described above, which binds to a target protein or polypeptide (e.g., SMARCA2, BRAHMA or BRM), which is ubiquitinated by a ubiquitin ligase and is chemically linked directly to the ULM group or through a linker moiety L; and L is a linker moiety as described above which may be present or absent and which chemically (covalently) links ULM to PTM, or a pharmaceutically acceptable salt, enantiomer, stereoisomer, solvate or polymorph thereof.

In certain embodiments, the linker group L is a group comprising one or more covalently connected structural units independently selected from:

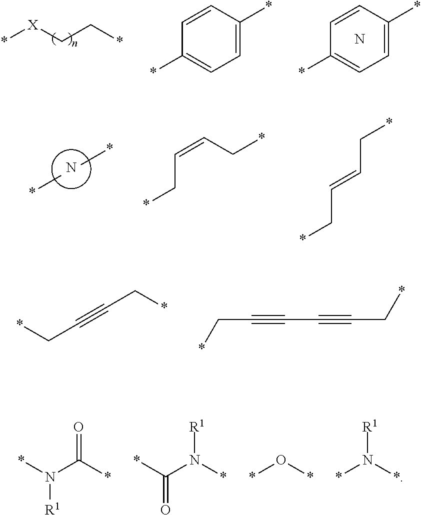

The X is selected from the group consisting of O, N, S, S(O) and SO₂; n is integer from 1 to 5; R^{L1} is hydrogen or alkyl,

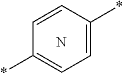

is a mono- or bicyclic aryl or heteroaryl optionally substituted with 1-3 substituents selected from alkyl, halogen, haloalkyl, hydroxy, alkoxy or cyano;

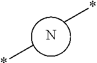

is a mono- or bicyclic cycloalkyl or a heterocyclyl optionally substituted with 1-3 substituents selected from alkyl, halogen, haloalkyl, hydroxy, alkoxy or cyano; and the phenyl ring fragment can be optionally substituted with 1, 2, or 3 substituents selected from the group consisting of alkyl, halogen, haloalkyl, hydroxy, alkoxy and cyano. In an embodiment, the linker group L comprises up to 10 covalently connected structural units, as described above.

In any aspect or embodiment described herein, the ULM group and PTM group is covalently linked to the linker group through any group that is appropriate and stable to the chemistry of the linker. In any aspect or embodiment described herein, the linker is independently covalently bonded to the ULM group and the PTM group through an amide, ester, thioester, keto group, carbamate (urethane), carbon or ether, each of which may be inserted anywhere on the ULM group and PTM group to provide maximum binding of the ULM group on the ubiquitin ligase and the PTM group on the target protein to be degraded. (It is noted that in certain embodiments, where the PTM group is a ULM group, the target protein for degradation may be the ubiquitin ligase itself.) In any aspect or embodiment described herein, the linker may be linked to an optionally substituted alkyl, alkylene, alkenyl or alkynyl group, an aryl group, or a heterocyclyl group on the ULM and/or PTM groups.

### Exemplary PTMs

In any aspect or embodiment of the present disclosure, the PTM group is a moiety, which binds to target proteins, such as Switch/Sucrose Non Fermentable (SWI/SNF)-Related, Matrix-Associated, Actin-Dependent Regulator of Chromatin, Subfamily A, Member 2 (SMARCA2) or BRM. Thus, in any aspect or embodiment described herein, the PTM group is any moiety that binds to SMARCA2 or BRM protein specifically (binds to the target protein SMARCA2, BRAHMA or BRM).

In certain embodiments, the compounds as described herein include a means for binding a target protein, e.g., Brm. As such, in certain aspects, the disclosure provides a bifunctional compound having a means for binding Brm, and a means for binding VHL and a means for chemically coupling the means for binding Brm to the means for binding VHL.

The compositions described below exemplify some of the members of small molecule target protein binding moieties. Such small molecule target protein binding moieties also include pharmaceutically acceptable salts, enantiomers, solvates and polymorphs of these compositions, as well as other small molecules that may target SMARCA2. These binding moieties are linked to the ubiquitin ligase binding moiety preferably through a linker in order to present a target protein (to which the protein target moiety is bound) in proximity to the ubiquitin ligase for ubiquitination and degradation. Any protein (e.g., SMARCA2, BRAHMA or BRM), which can bind to a protein target moiety or PTM group and acted on or degraded by a ubiquitin ligase is a target protein according to the present disclosure.

The present disclosure may be used to treat a number of disease states and/or conditions; including any disease state and/or condition in which proteins are dysregulated (e.g., SMARCA4-deficiency/mutation) and where a patient would benefit from the degradation and/or inhibition of proteins, such as SMARCA2, BRAHMA or BRM.

In an additional aspect, the description provides therapeutic compositions comprising an effective amount of a compound as described herein or salt form thereof, and a pharmaceutically acceptable carrier, additive or excipient, and optionally an additional bioactive agent. The therapeutic compositions modulate protein degradation in a patient or subject, for example, an animal such as a human, and can be used for treating or ameliorating disease states or conditions which are modulated through the degraded protein. In certain embodiments, the therapeutic compositions as described herein may be used to effectuate the degradation of proteins of interest for the treatment or amelioration of a disease, e.g., cancer such as at least one of a SWI/SNF associated cancer, a SMARCA4-mutation associated cancer, a SMARCA4-deficient cancer, or a cancer with decreased expression of SMARCA4 relative to normal SMARCA4 expression (e.g., decreased expression relative to the expression of non-mutated SMARCA4 or SMARCA4 in a similarly situated non-cancerous cell with wildtype SMARCA4), including lung cancer or non-small cell lung cancer. In any aspect or embodiment described herein, the disease is at least one of SWI/SNF associated cancer, a cancer with a SMARCA4 mutation, a cancer with a SMARCA4-deficiency, or a combination thereof, which may be lung cancer or a non-small cell lung cancer.

In certain additional embodiments, the therapeutic compositions as described herein may be used to effectuate the degradation of proteins of interest for the treatment or amelioration of a disease, e.g., cancer such as at least one of a SWI/SNF associated cancer, a SMARCA2-associated cancer or a cancer with normal or over-expression of SMARCA2.

In alternative aspects, the present disclosure relates to a method for treating a disease state or ameliorating the symptoms of a disease or condition in a subject in need thereof by degrading a protein or polypeptide through which a disease state or condition is modulated comprising administering to said patient or subject an effective amount, e.g., a therapeutically effective amount, of at least one compound as described hereinabove, optionally in combination with a pharmaceutically acceptable carrier, additive or excipient, and optionally an additional bioactive agent, wherein the composition is effective for treating or ameliorating the disease or disorder or symptom thereof in the subject. The method according to the present disclosure may be used to treat a large number of disease states or conditions including cancer, by virtue of the administration of effective amounts of at least one compound described herein. The disease state or condition may be a disease caused by a microbial agent or other exogenous agent such as a virus, bacteria, fungus, protozoa or other microbe or may be a disease state, which is caused by overexpression of a protein, which leads to a disease state and/or condition.

In another aspect, the description provides methods for identifying the effects of the degradation of proteins of interest in a biological system using compounds according to the present disclosure.

The term “target protein” is used to describe a protein or polypeptide, which is a target for binding to a compound according to the present disclosure and degradation by ubiquitin ligase hereunder. Such small molecule target protein binding moieties also include pharmaceutically acceptable salts, enantiomers, solvates and polymorphs of these compositions, as well as other small molecules that may target a protein of interest. These binding moieties are linked to at least one ULM group (e.g. VLM) through at least one linker group L.

The protein target may be used in screens that identify compound moieties which bind to the protein and by incorporation of the moiety into compounds according to the present disclosure, the level of activity of the protein may be altered for therapeutic end result.

The term “protein target moiety” or PTM is used to describe a small molecule which binds to a target protein or other protein or polypeptide of interest, such as SMARCA2 or BRM, and places/presents that protein or polypeptide in proximity to an ubiquitin ligase such that degradation of the protein or polypeptide by ubiquitin ligase may occur. The compositions described below exemplify some of the members of the small molecule target proteins.

In any aspect or embodiment described herein, the PTM of the present disclosure has a chemical structure represented by:

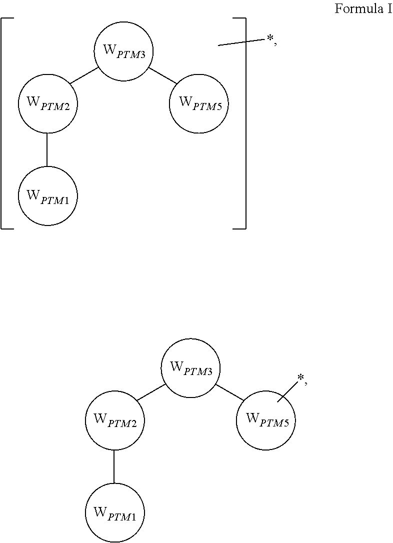

wherein
W_{PTM1} is an optionally substituted 5-6-membered aryl or heteroaryl ring (e.g., a 5-6 member aryl or heteroaryl substituted with 0, 1, 2, or 3 substituents selected from hydroxy, halogen, alkoxy, alkyl, haloalkyl, phosphate, amino, alkylamino, cyano or combination thereof);
W_{PTM2} is an optionally substituted 5-6-membered aryl or heteroaryl ring (e.g., a 5-6 membered aryl or heteroaryl substituted with 0, 1, 2, or 3 substituents selected from hydroxy, halogen, alkoxy, alkyl, haloalkyl, amino, alkylamino and cyano);
W_{PTM3} is an optionally substituted 3-9-membered aryl or heteroaryl ring (e.g., an optionally substituted 5-6-membered aryl or heteroaryl ring, or a 3-9 or 5-6 membered aryl or heteroaryl substituted with 0, 1, 2, or 3 substituents selected from hydroxy, halogen, alkoxy, alkyl, haloalkyl, amino, alkylamino and cyano), or an optionally substituted 4-10 membered cycloalkyl or heterocyclyl, such as an optionally substituted bridged bicycloalkyl and bridged biheterocyclyl rings (e.g. a 4-10 membered cycloalkyl or heterocyclyl substituted with 0, 1, or 2 substituents selected from hydroxy, halogen, alkoxy, alkyl, haloalkyl, amino, alkylamino and cyano);
W_{PTM5} is absent (such that W_{PTM3} is connected directly to L (linker) or ULM) or an optionally substituted alkyl, an optionally substituted 5-6-membered cycloalkyl, heterocycle, aryl or heteroaryl ring (e.g. a 5-6 membered cycloalkyl, heterocycle, aryl or heteroaryl substituted with 0, 1, or 2 substituents selected from hydroxy, halogen, alkoxy, alkyl, haloalkyl, amino, alkylamino and cyano); and
[symbol] is the attachment point to the, linker, ULM group, or VLM group. In any aspect or embodiment described herein, W_{PTM5} is a piperidine.

In certain embodiments, W_{PTM1} comprises a phosphate substitution.

In any aspect or embodiment described herein, the PTM of the present disclosure is represented by Formula I, wherein at least one of:
W_{PTM1} is an optionally substituted phenyl or a pyridyl (e.g., substituted as described herein, such as a phenyl substituted with a hydroxy or phosphate substituent with or without an additional optional substituent selected as described herein, e.g., substituted with 0, 1, 2, or 3 substituents selected from hydroxy, halogen, alkoxy, alkyl, haloalkyl, amino, alkylamino, cyano or combination thereof);
W_{PTM2} is an optionally substituted 6-membered heteroaryl ring (e.g., substituted as described herein, such as a pyridazine substituted with amino group);
W_{PTM3} is an optionally substituted 5-6-membered heteroaryl (e.g., a pyrazole, pyrrole, imidazole, oxazole, oxadiazole, or triazole);
W_{PTM5} is as described in any aspect or embodiment described herein (e.g., W_{PTM5} may be absent or a pyridine ring); or
a combination thereof.

In any aspect or embodiment described herein, for example, an embodiment that includes a PTM of Formula I, W_{PTM3} is a pyrazole or a 6-8-membered heterocyclyl (e.g., a piperazine or a diazabicyclooctane).

In any aspect or embodiment described herein, the PTM of the present disclosure has a chemical structure represented by:

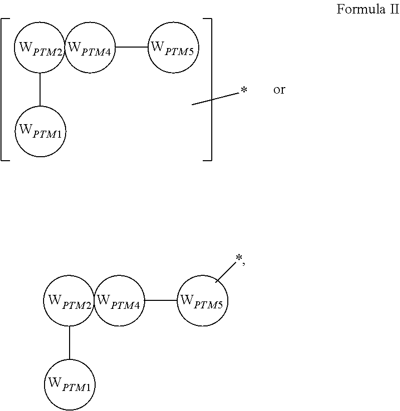

wherein
W_{PTM1}, W_{PTM2}, and W_{PTM5} are as described in any other aspect or embodiment described herein (e.g., W_{PTM5} may or may not be present, such that WPTM4 may be connected directly to L (linker) or the ULM);
W_{PTM4} is an optionally substitute 3-7 cycloalkyl or heterocyclyl (e.g., optionally substituted 5-7 cycloalkyl or heterocyclyl or a 5-7 cycloalkyl or heterocyclyl substituted with 0, 1, 2, or 3 substituents selected from hydroxy, halogen, alkoxy, alkyl, haloalkyl, amino, alkylamino and cyano) that is fused with the W_{PTM2} ring; and
[symbol] is the attachment point to the linker, ULM group, or VLM group.

In any aspect or embodiment described herein, the PTM of the present disclosure is represented by Formula II, wherein W_{PTM1}, W_{PTM2} and W_{PTM5} are as described in any of the aspects or embodiment described herein, and W_{PTM4} is a piperazine ring. For example, in any aspect or embodiment described herein, W_{PTM2} and W_{PTM4} of Formula II taken together constitute a dihydropirazino[2,3-e]pyridazine as shown:

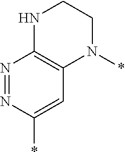 [SMILES: *c1cc2c(nn1)NCCN2*]

In any aspect or embodiment described herein, the PTM of the present disclosure has a chemical structure represented by:

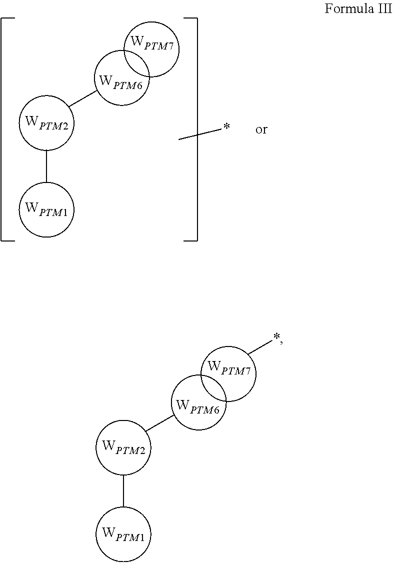

wherein:
W_{PTM1} and W_{PTM2} are as described in any aspect or embodiment described herein;
W_{PTM6} and W_{PTM7} are independently an optionally 4-7 cycloalkyl or heterocyclyl (e.g., each is independently a 4-7 cycloalkyl or heterocyclyl substituted with 0, 1, or 2 substituents selected from hydroxy, halogen, alkoxy, alkyl, haloalkyl, amino, alkylamino and cyano), and the rings of W_{PTM6} and W_{PTM7} are fused or linked via a spiro connection; and
[symbol] is the attachment point to the, linker, ULM group, or VLM group.

In any aspect or embodiment described herein, the PTM of the present disclosure has a chemical structure represented by formula III, wherein W_{PTM1} and W_{PTM2} are each independently selected as described in any aspect or embodiment described herein (e.g., W_{PTM1} is a phenyl substituted with a hydroxy substituent with or without an additional optional substituent selected as described herein, WP™₂ is a pyridazine substituted with amino group), and W_{PTM6} and W_{PTM7} are a spirocyclic ring system, for example, a spirocyclic ring selected from:

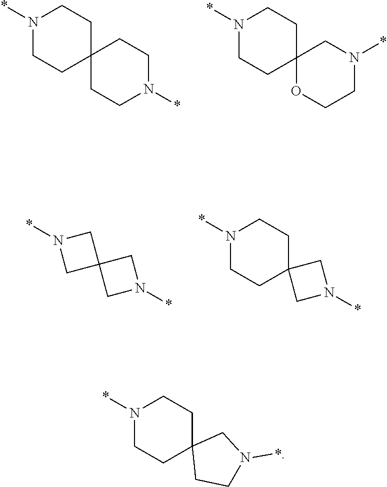

In any aspect or embodiment described herein, the PTM of the the present disclosure is represented by:

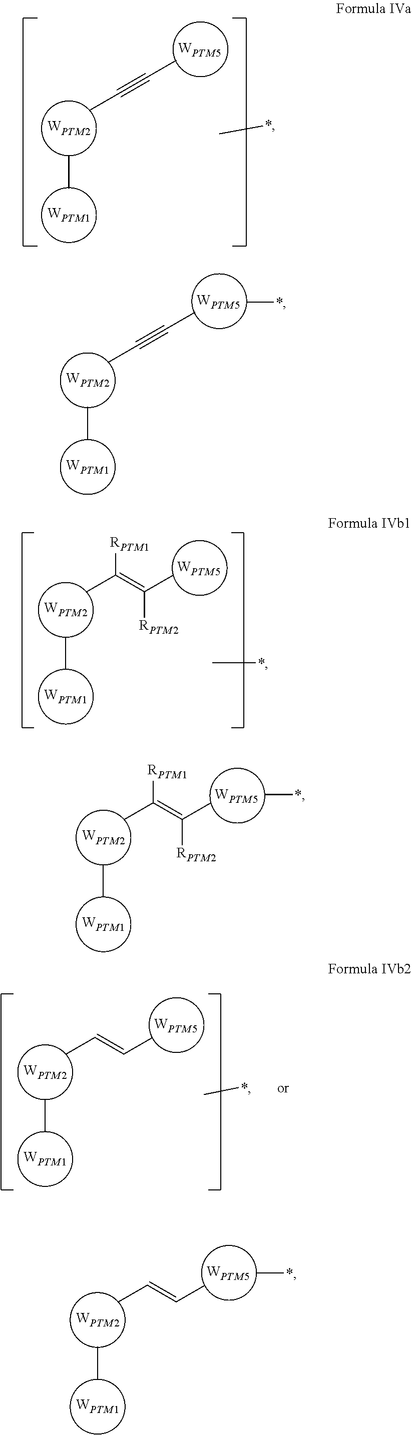

W_{PTM1} is as described in any other aspect or embodiment described herein, such as W_{PTM1} is an optionally substituted 5-6-membered aryl or heteroaryl ring (e.g., a 5-6 member aryl or heteroaryl substituted with 0, 1, 2, or 3 substituents selected from hydroxy, halogen, alkoxy, alkyl, haloalkyl, amino, alkylamino and cyano);
W_{PTM2} is as described in any other aspect or embodiment described herein, such as W_{PTM2} is an optionally substituted 5-6-membered aryl or heteroaryl ring (e.g., a 5-6 membered aryl or heteroaryl substituted with 0, 1, 2, or 3 substituents selected from hydroxy, halogen, alkoxy, alkyl, haloalkyl, amino, alkylamino and cyano);
W_{PTM5} is as described in any other aspect or embodiment described herein, such as W_{PTM5} is absent or an optionally substituted alkyl, an optionally substituted 5-6-membered cycloalkyl, heterocycle, aryl or heteroaryl ring (e.g. a 5-6 membered cycloalkyl, heterocycle, aryl or heteroaryl substituted with 0, 1, or 2 substituents selected from hydroxy, halogen, alkoxy, alkyl, haloalkyl, amino, alkylamino and cyano) with the proviso that a heteroatom is not directly connected to the carbon atom of the carbon-carbon double bond, or a carbon-carbon triple bond;
L_{PTM} is selected from the group consisting of: an alkyne or an alkene optionally substituted with 1-2 substituents independently selected from methyl, fluoro or haloalkyl; a C1-C2 alkyl optionally substituted with 1-2 substituents selected from methyl, fluoro, or haloalkyl; or a cyclopropyl optionally substituted with 1-2 substituents selected from methyl, fluoro, or haloalkyl;
R_{PTM1} and R_{PTM2} are individually a H, halogen, OH, C1-C3 alkyl, C1-C3 haloalkyl, or C1-C3 alkoxy; and
[symbol] is the attachment point to the, linker, ULM group, or VLM group

In any aspect or embodiment described herein, R_{PTM1} and R_{PTM2} are individually a H, halogen, C1-C3 alkyl, or C1-C3 haloalkyl.

In any aspect or embodiment described herein, W_{PTM5} is an optionally substituted alkyl, an optionally substituted 5-6-membered cycloalkyl, heterocycle, aryl or heteroaryl ring (e.g. a 5-6 membered cycloalkyl, heterocycle, aryl or heteroaryl substituted with 0, 1, or 2 substituents selected from hydroxy, halogen, alkoxy, alkyl, haloalkyl, amino, alkylamino and cyano) with the proviso that a heteroatom is not directly connected to the carbon atom of the carbon-carbon double bond, or a carbon-carbon triple bond.

In any aspect or embodiment described herein, the PTM of the present disclosure has the chemical structure represented by Formula IV, wherein at least one of:
W_{PTM1} is a phenyl substituted with a hydroxy or phosphate substituent with or without an additional optional substituent selected as described herein;
W_{PTM2} is a pyridazine substituted with amino group;
W_{PTM5} is absent, a pyrazole ring, or a pyridine ring; or
a combination thereof.

In any aspect or embodiment described herein, the PTM of the present disclosure is represented by:

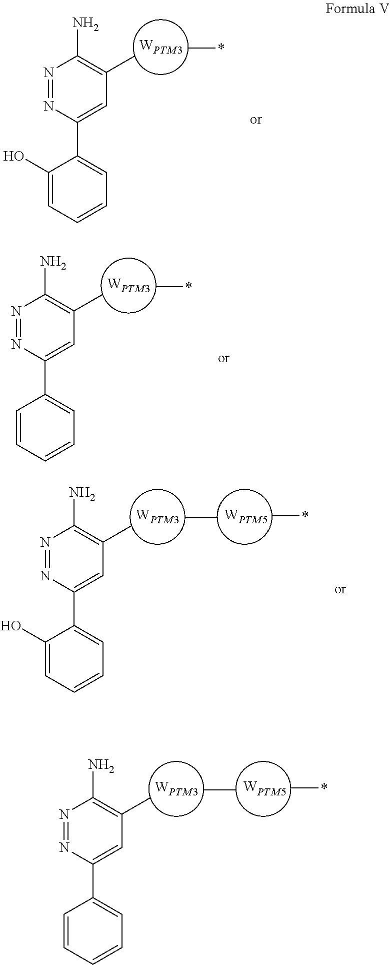

or a pharmaceutically acceptable salt thereof, wherein:
W_{PTM3} is absent or an optionally substituted 5-6-membered heteroaryl, an optionally substituted 4-9 cycloalkyl or heterocyclyl ring, an optionally substituted bridged bicycloalkyl and bridged biheterocyclyl ring; and
W_{PTM5} is an optionally substituted 5-6-membered heteroaryl or aryl, e.g., pyridine, or pyridazine.

In any aspect or embodiment described herein, the PTM of the present disclosure is represented by:

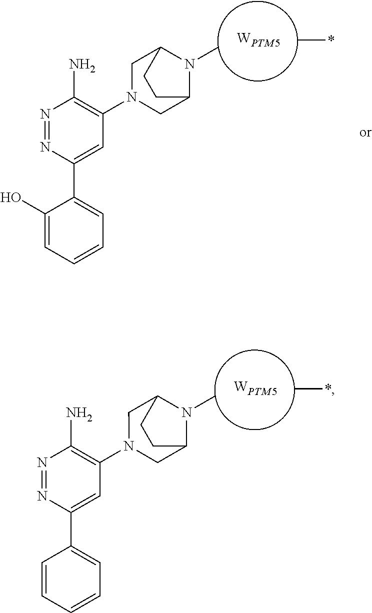

or a pharmaceutically acceptable salt thereof, wherein: W_{PTM5} is phenyl, pyridine, pyrimidine or pyrazine.

In any aspect or embodiment described herein, the PTM of the present disclosure is represented by:

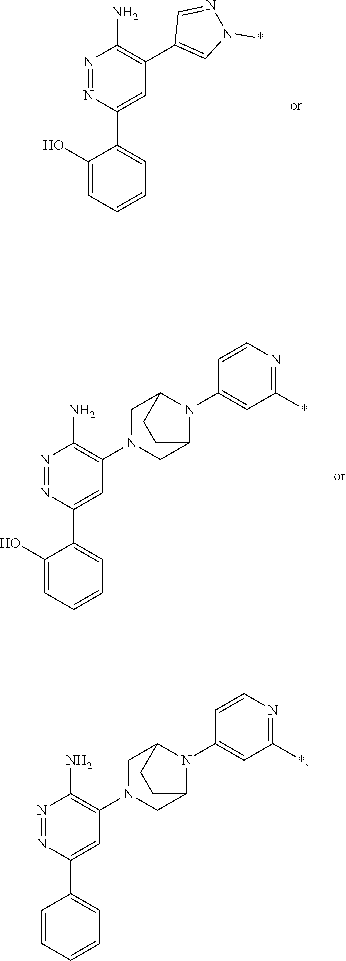

or a pharmaceutically acceptable salt thereof.

In any aspect or embodiment described herein, the PTM of the present disclosure is represented by:

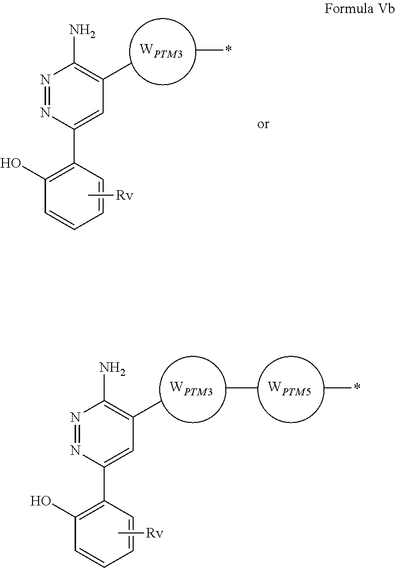

or a pharmaceutically acceptable salt thereof, wherein:
W_{PTM3} is an optionally substituted 5-6-membered heteroaryl, an optionally substituted 4-9 cycloalkyl or heterocyclyl ring, an optionally substituted bridged bicycloalkyl and bridged biheterocyclyl ring; W_{PTM5} is an optionally substituted 5-6-membered heteroaryl or aryl, e.g., pyridine, or pyridazine; Rv is 0, 1, 2 or 3 substituents independently selected from hydroxy, halogen, alkoxy, alkyl, haloalkyl, phosphate, amino, alkylamino, cyano or a combination thereof.

In certain embodiments, the hydroxyl group is modified with a phosphate group (i.e., a phosphoester group).

In any aspect or embodiment described herein, the PTM of the present disclosure has a chemical structure represented by:

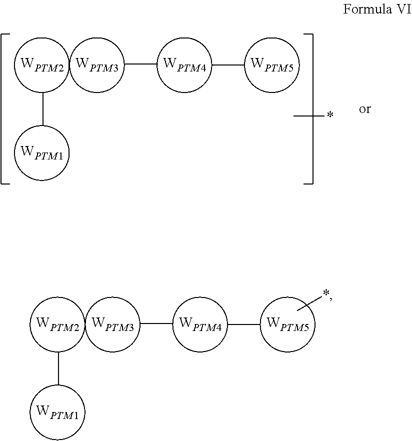

wherein:
W_{PTM1} and W_{PTM2} are as described in any other aspect or embodiment described herein (e.g., W_{PTM5} may or may not be present, such that WPTM4 may be connected directly to L (linker) or the ULM);
W_{PTM3} is absent or an optionally substituted 5-7 cycloalkyl or heterocyclyl (e.g., 5-7 cycloalkyl or heterocyclyl substituted with 0, 1, 2, or 3 substituents selected from hydroxy, halogen, alkoxy, alkyl, haloalkyl, amino, alkylamino and cyano) that is fused with the W_{PTM2} ring; W_{PTM4} is an optionally substituted 3-7-membered aryl or heteroaryl ring (e.g., optionally substituted 5-7 cycloalkyl or heterocyclyl, or a 3-7 or a 5-6 membered aryl or heteroaryl substituted with 0, 1, 2, or 3 substituents selected from hydroxy, halogen, alkoxy, alkyl, haloalkyl, amino, alkylamino and cyano), or an optionally substituted 4-9 cycloalkyl or heterocyclyl, such as an optionally substituted bridged bicycloalkyl and bridged biheterocyclyl rings (e.g. a 4-9 or 5-7 cycloalkyl or heterocyclyl substituted with 0, 1, or 2 substituents selected from hydroxy, halogen, alkoxy, alkyl, haloalkyl, amino, alkylamino and cyano);
W_{PTM5} is absent (such that W_{PTM4} is connected directly to L (linker) or ULM) or an optionally substituted alkyl, an optionally substituted 5-6-membered cycloalkyl, heterocycle, aryl or heteroaryl ring (e.g. a 5-6 membered cycloalkyl, heterocycle, aryl or heteroaryl substituted with 0, 1, or 2 substituents selected from hydroxy, halogen, alkoxy, alkyl, haloalkyl, amino, alkylamino and cyano), e.g., an optionally substituted pyrazole ring, or a pyridine ring; and
[symbol] is the attachment point to the, linker, ULM group, or VLM group.

In any aspect or embodiment described herein, the PTM of the present disclosure has a chemical structure represented by:

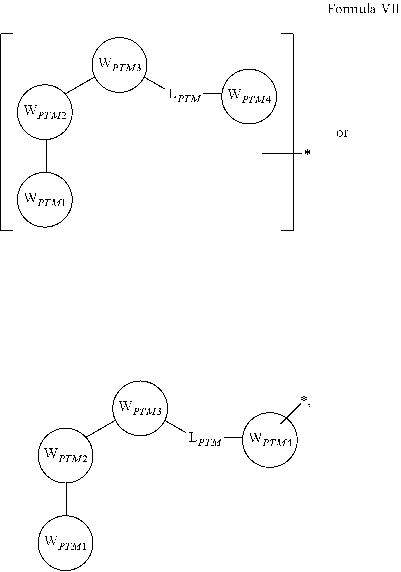

wherein:
W_{PTM1} is a 5-6-membered aryl or heteroaryl ring optionally substituted with 0, 1, 2, or 3 substituents selected from hydroxy, halogen, alkoxy, alkyl, haloalkyl, amino, phosphate, alkylamino, cyano or a combination thereof;
W_{PTM2} is a 5-6-membered aryl or heteroaryl ring optionally substituted with 0, 1, 2, or 3 substituents selected from hydroxy, halogen, alkoxy, alkyl, haloalkyl, amino, alkylamino, cyano or a combination thereof;
W_{PTM3} is absent or a 3-9-membered aryl or heteroaryl ring optionally substituted with 0, 1, 2, or 3 substituents selected from hydroxy, halogen, alkoxy, alkyl, haloalkyl, amino, alkylamino, cyano or a combination thereof, a 3-9 membered cycloalkyl or heterocyclyl optionally substituted with 0, 1, or 2 substituents selected from hydroxy, halogen, alkoxy, alkyl, haloalkyl, amino, alkylamino, cyano or a combination thereof, or a bridged bicycloalkyl or bridged biheterocyclyl optionally substituted with 0, 1, or 2 substituents selected from hydroxy, halogen, alkoxy, alkyl, haloalkyl, amino, alkylamino, cyano or a combination thereof;
W_{PTM4} is a 3-7 membered cycloalkyl or heterocyclyl optionally substituted with 0, 1, 2, or 3 substituents selected from hydroxy, halogen, alkoxy, alkyl, haloalkyl, amino, phosphate, alkylamino, cyano or a combination thereof;
L_{PTM} is 0 or C1-C6 alkyl optionally substituted with an ═O, C1-C4 alkyl or C1-C3 alkoxy; and
[symbol] is the attachment point to the, linker, ULM group, or VLM group.

In any aspect or embodiment described herein, the PTM is selected from:

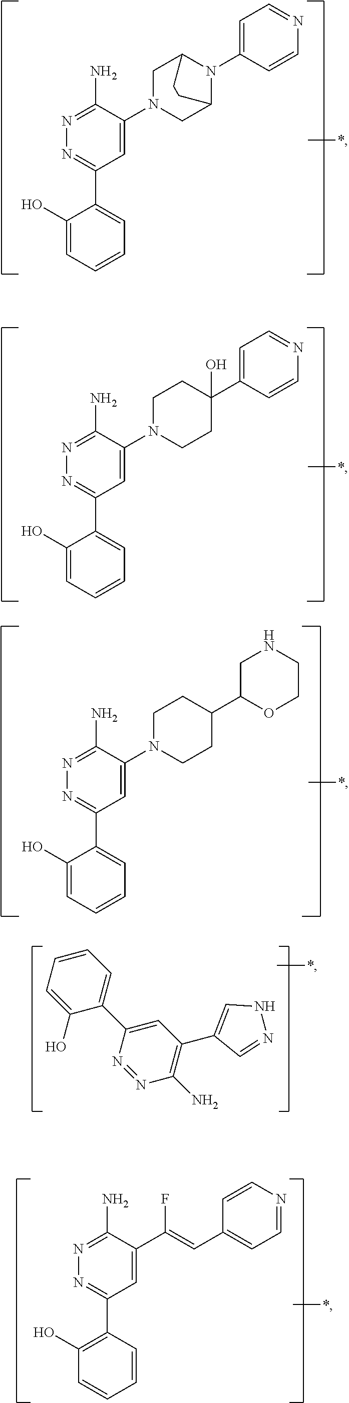

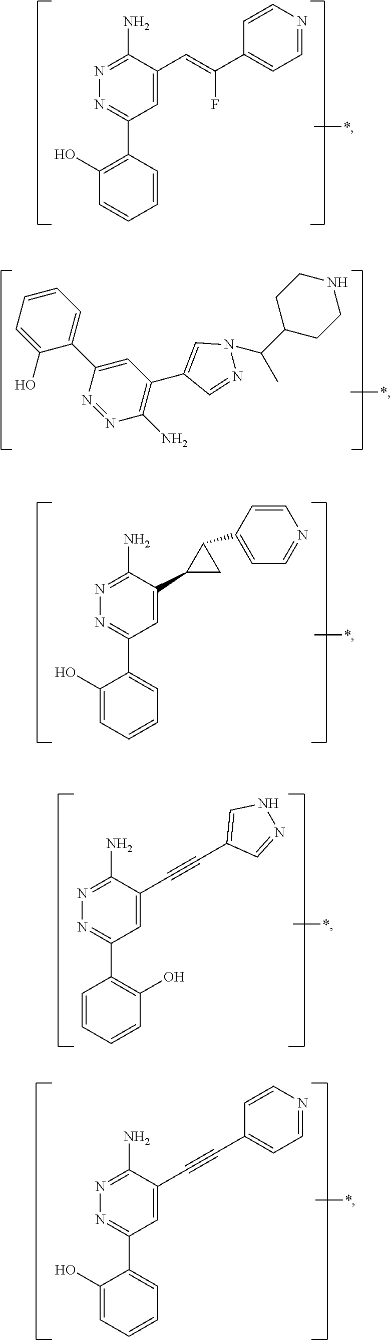

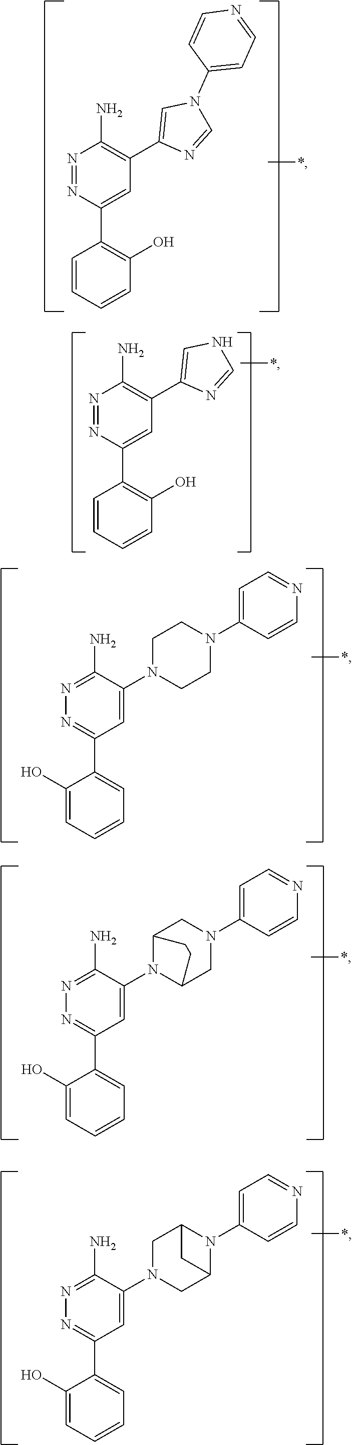

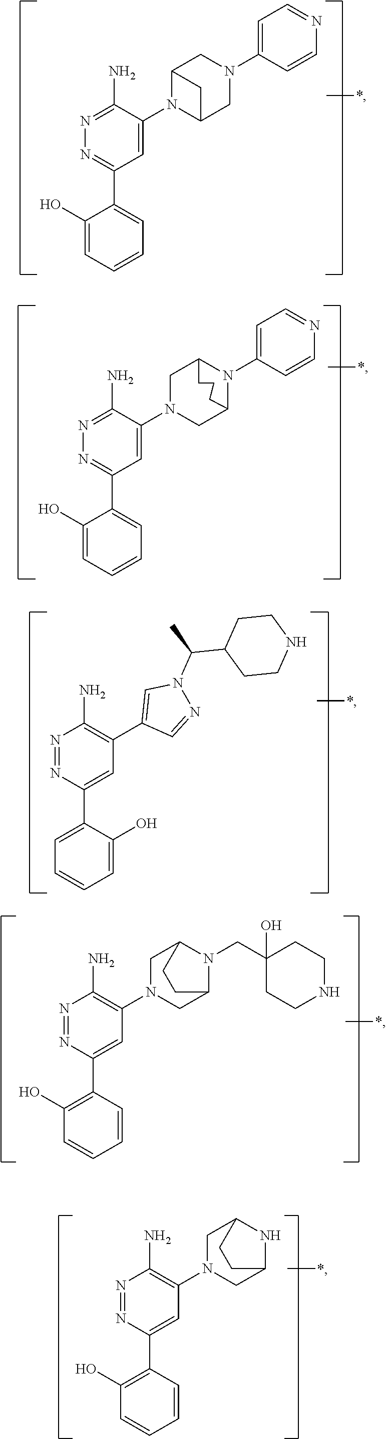

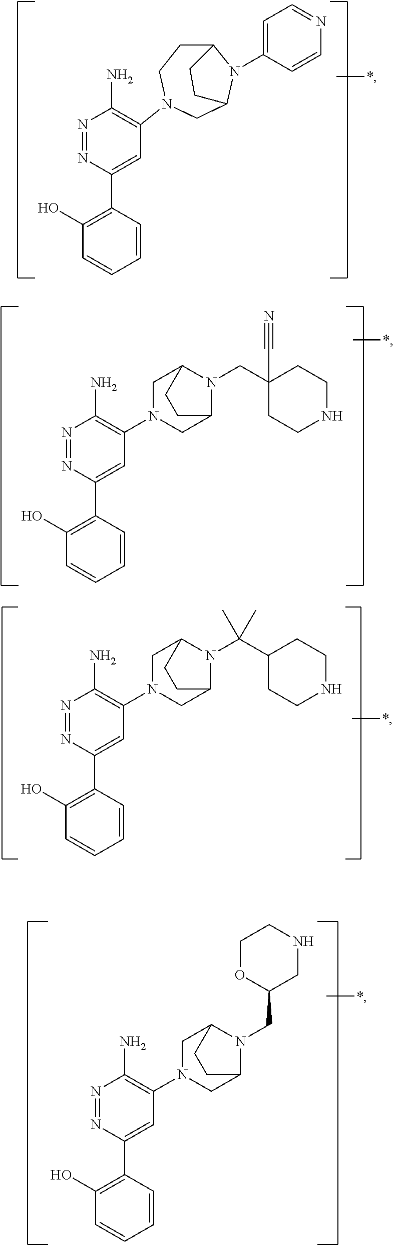

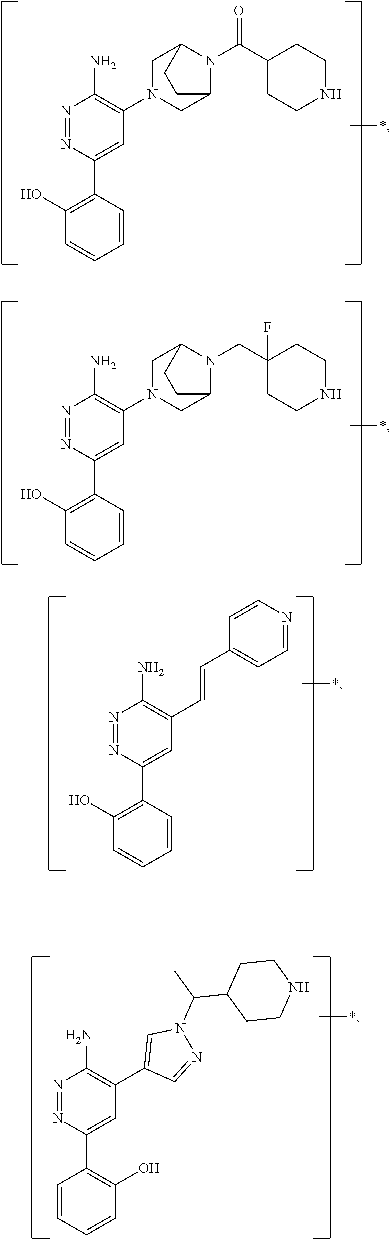

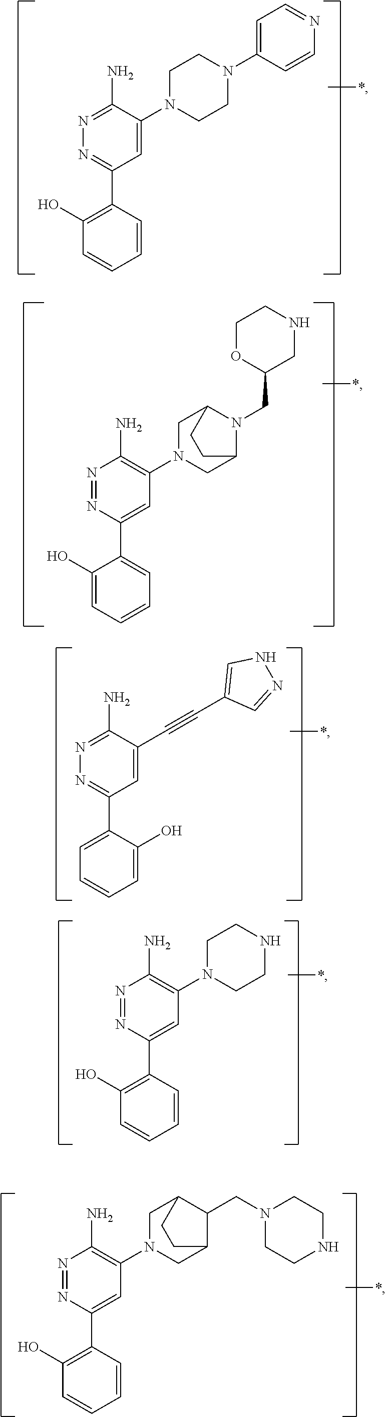

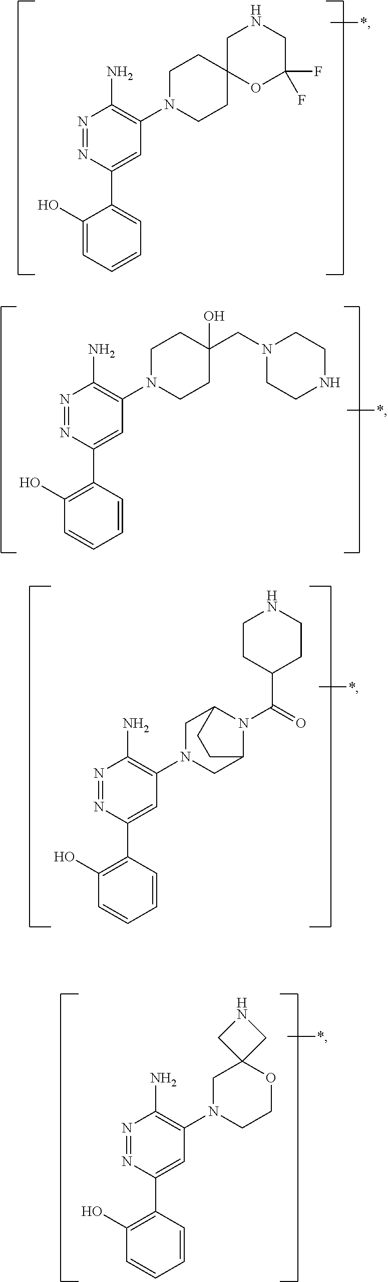

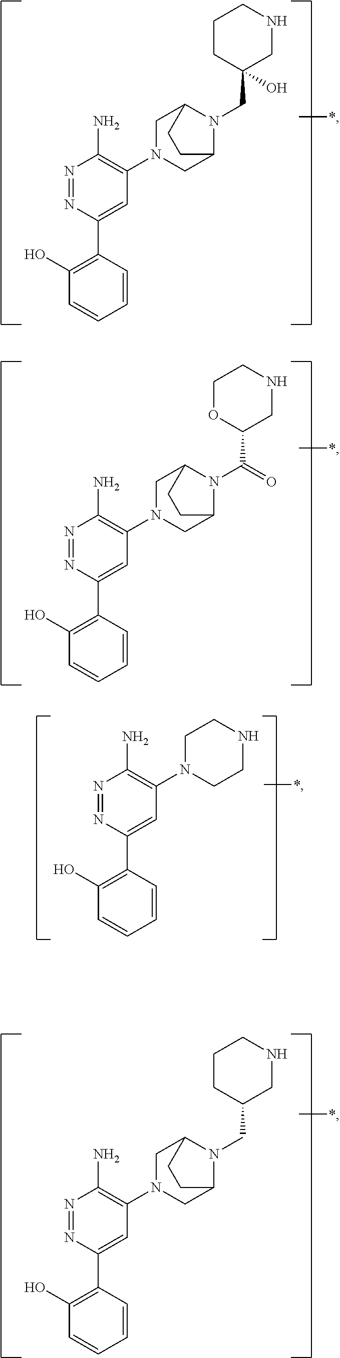

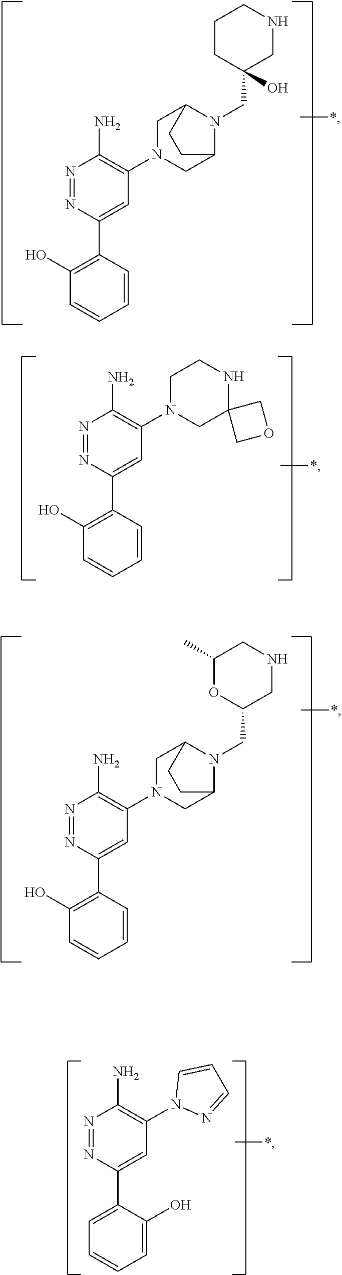

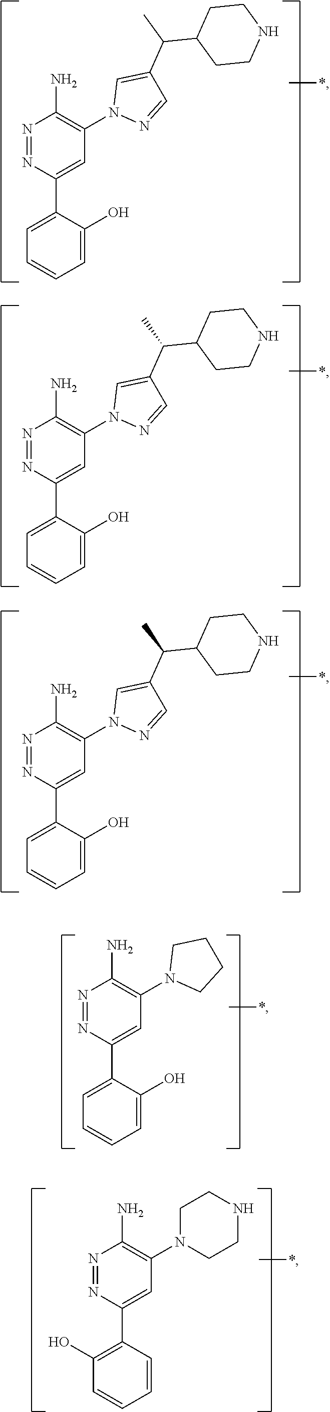

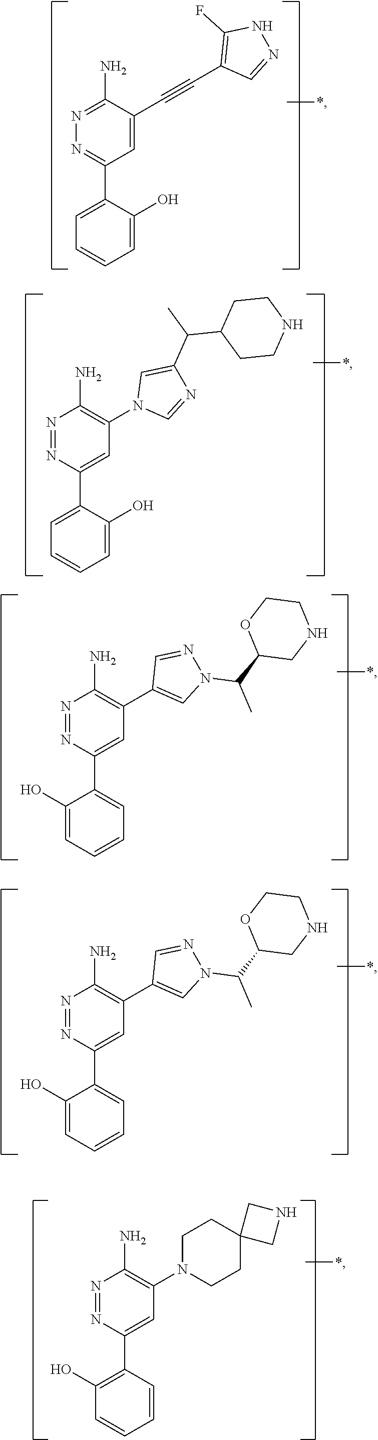

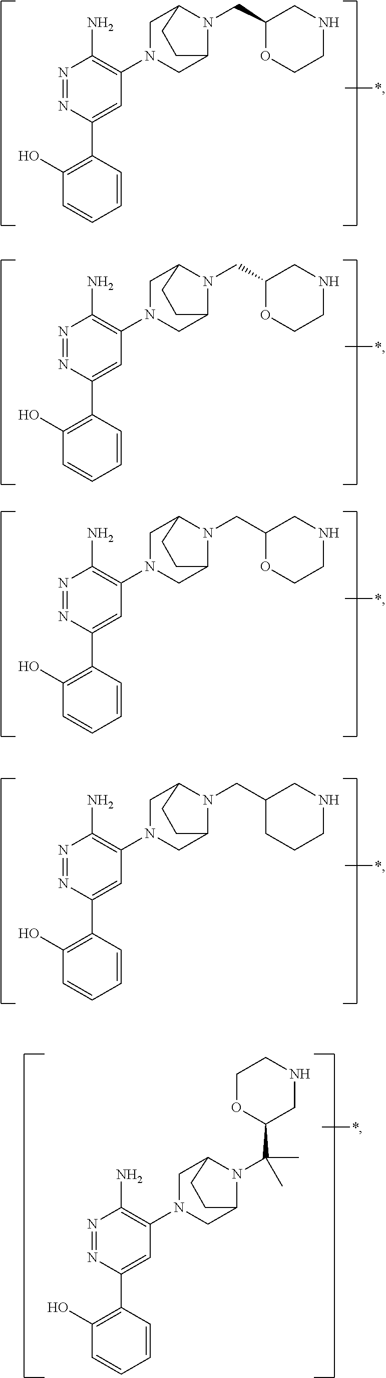

[chemical structure]

wherein [symbol] is the attachment point to the chemical linking moiety.

In any aspect or embodiment described herein, the PTM is selected from:

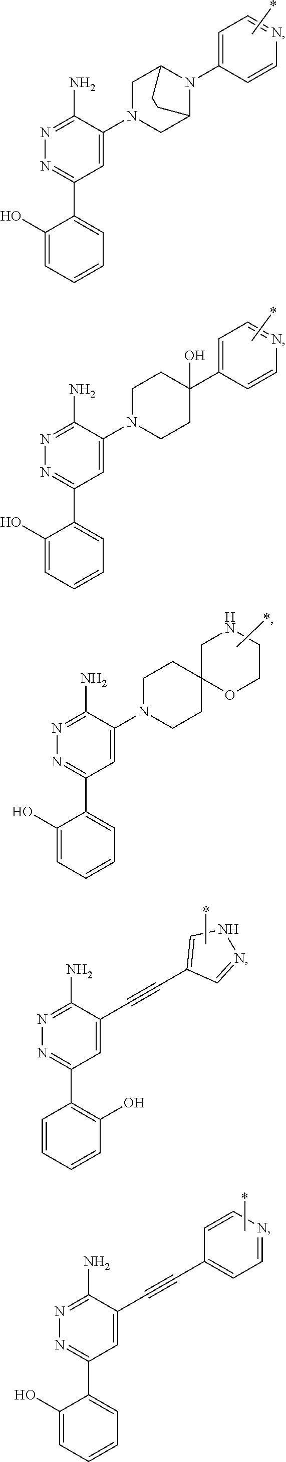

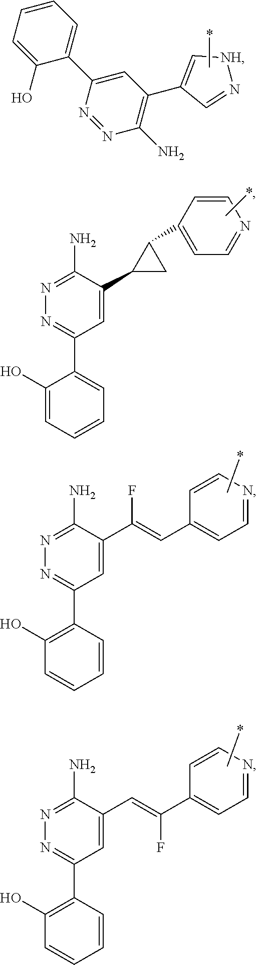

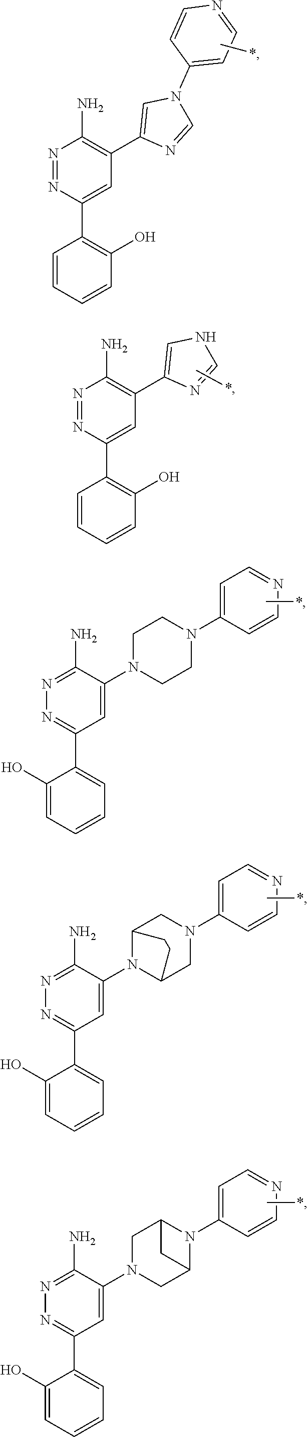

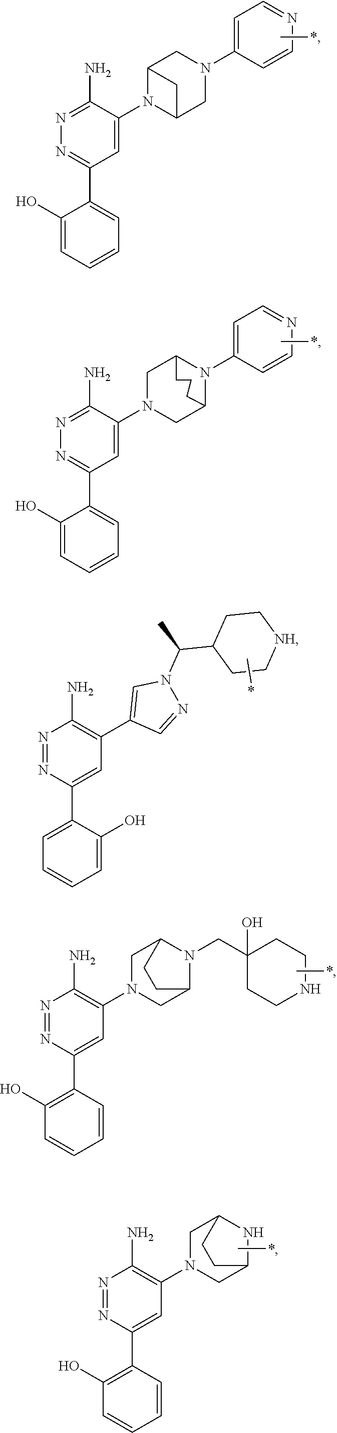

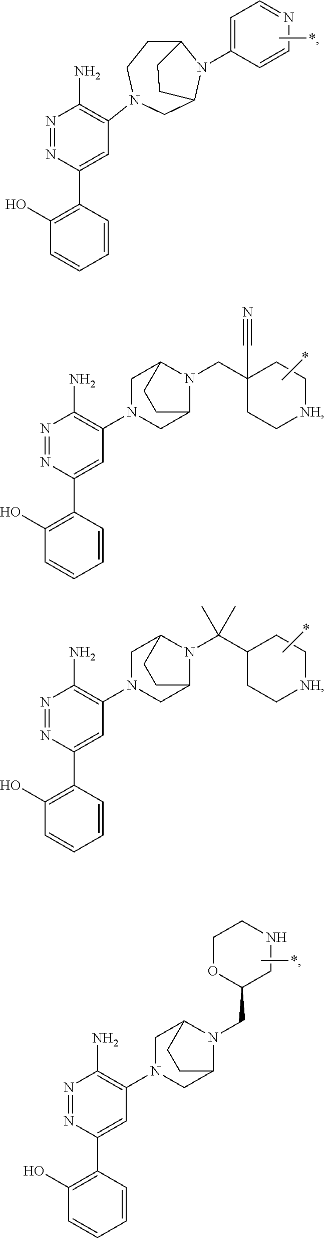

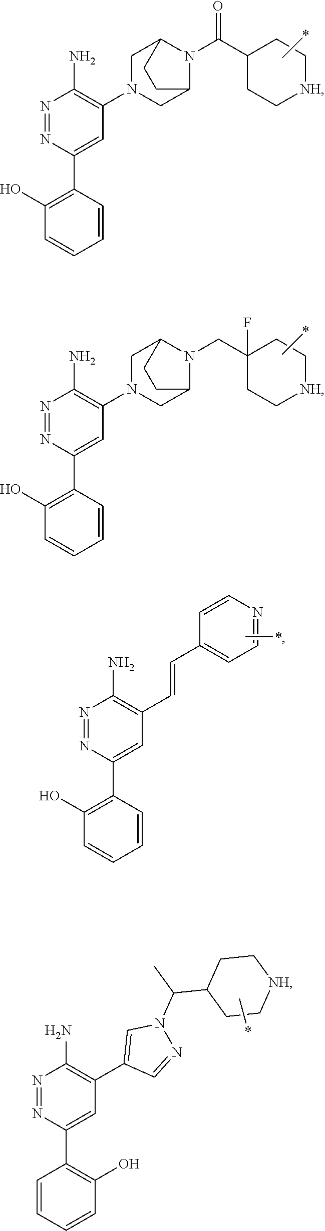

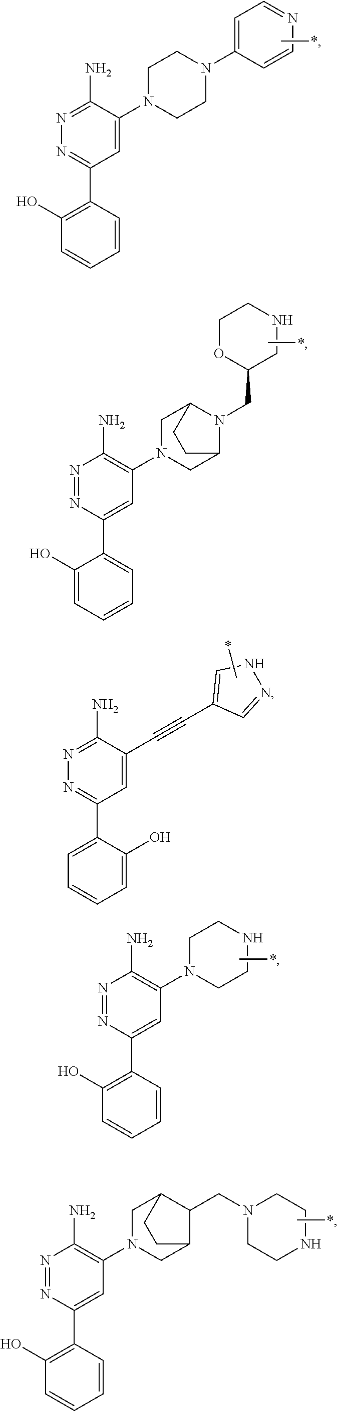

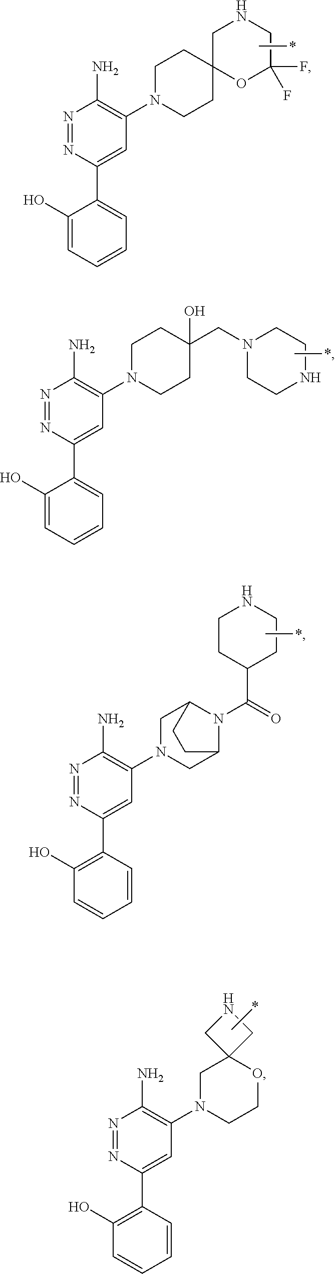

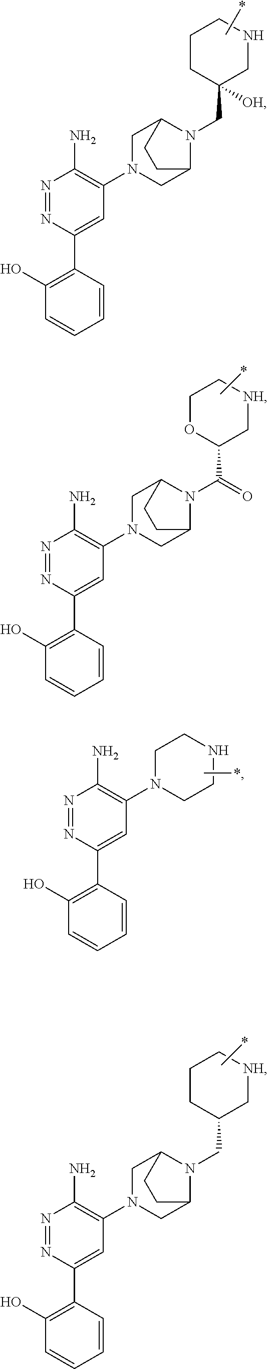

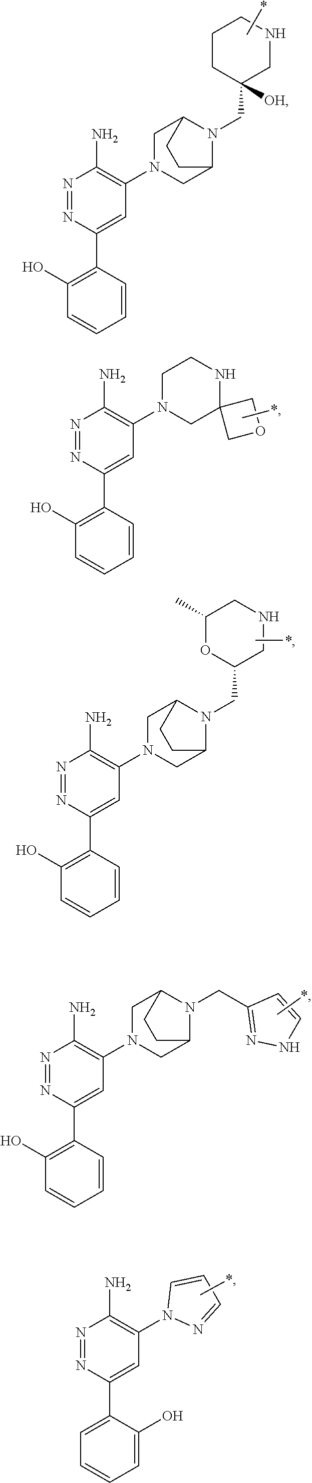

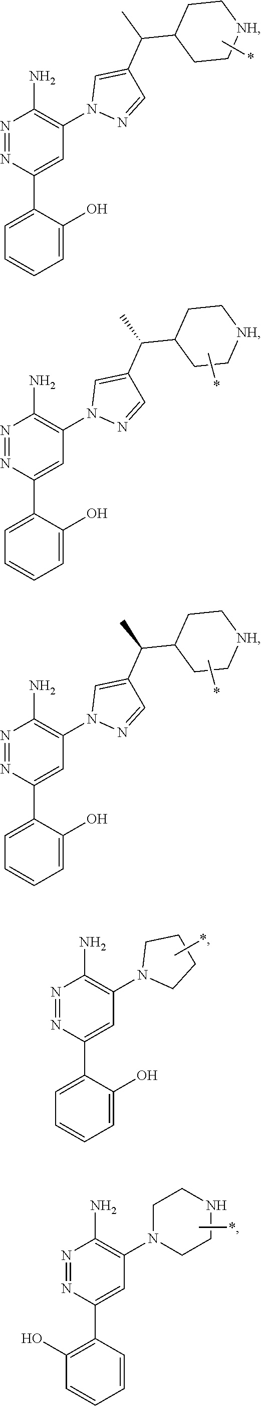

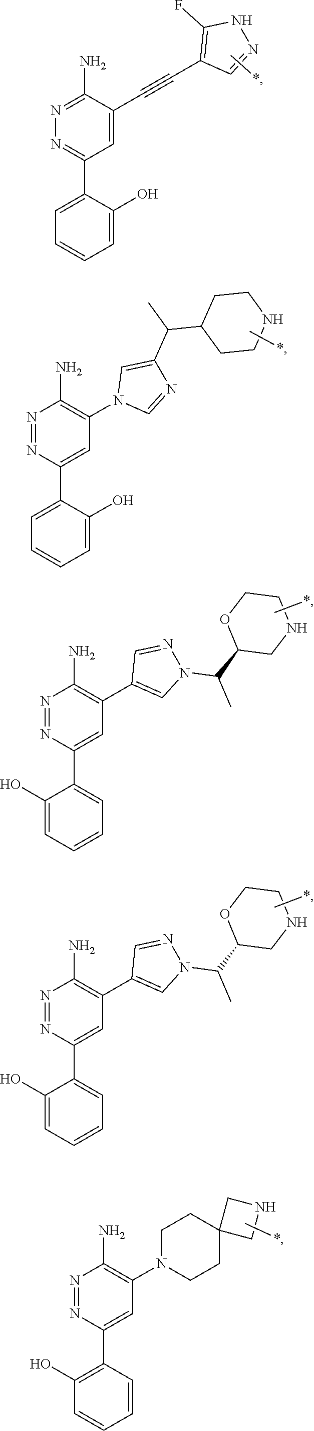

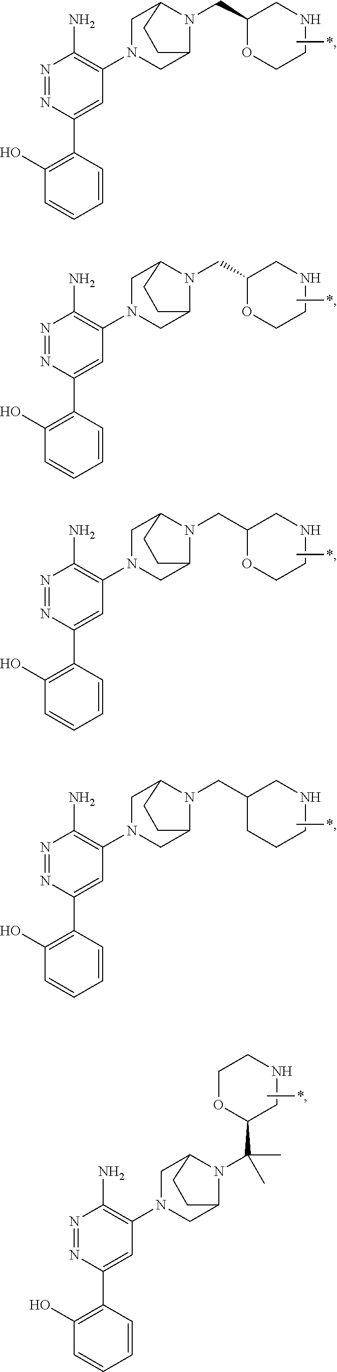

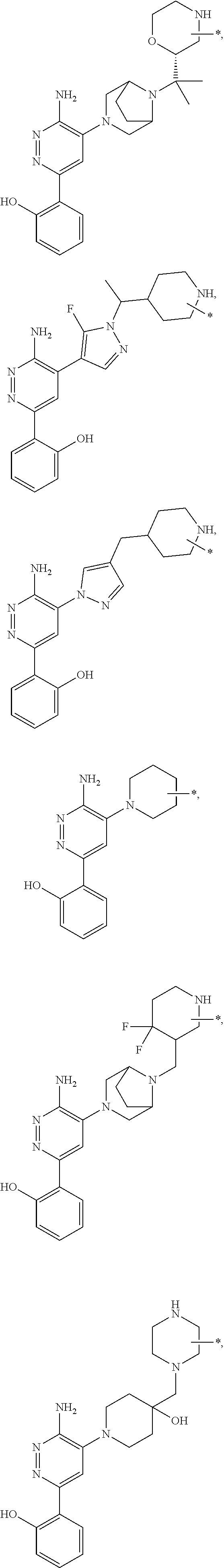

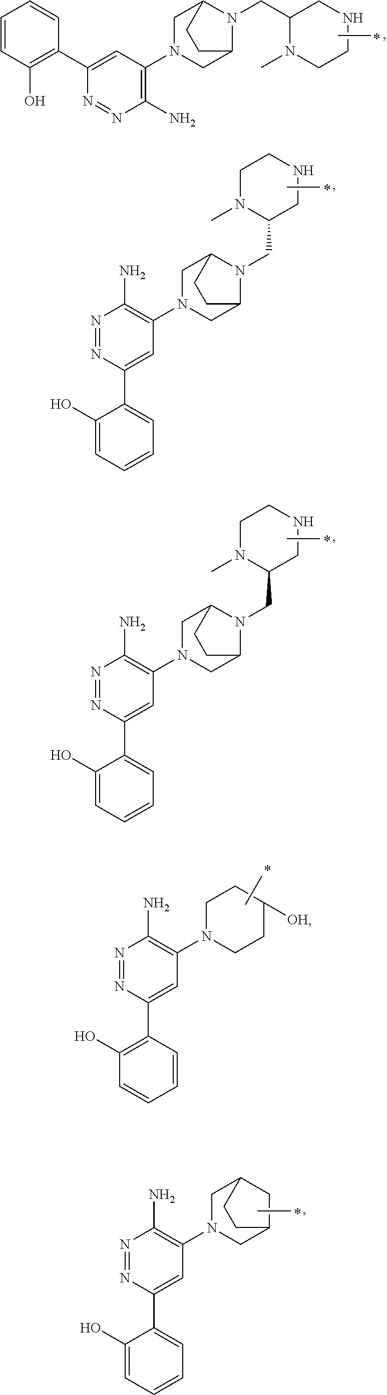

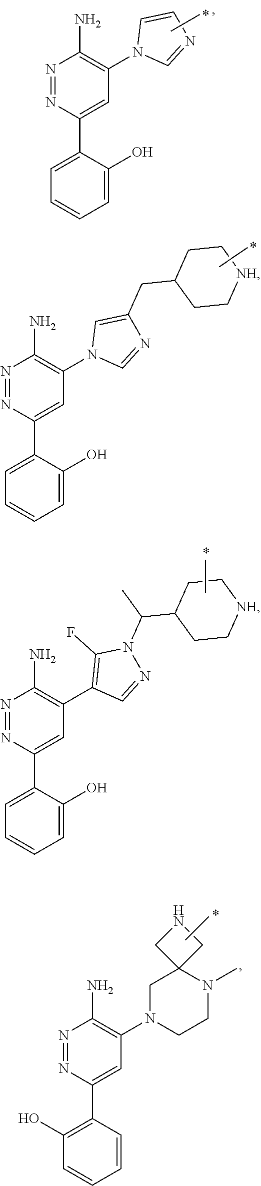

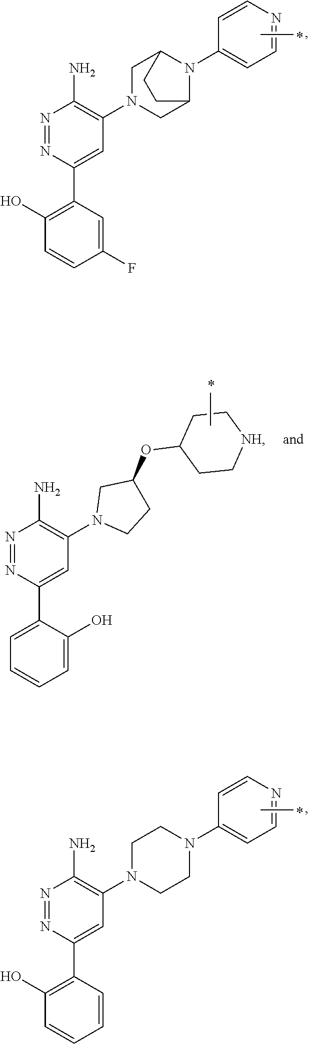

wherein [symbol] is the attachment point to the chemical linking moiety (e.g., the chemical linker group is attached to a carbon or heteroatom of the indicated ring).

In any aspect or embodiment described herein, the PTM is selected from:

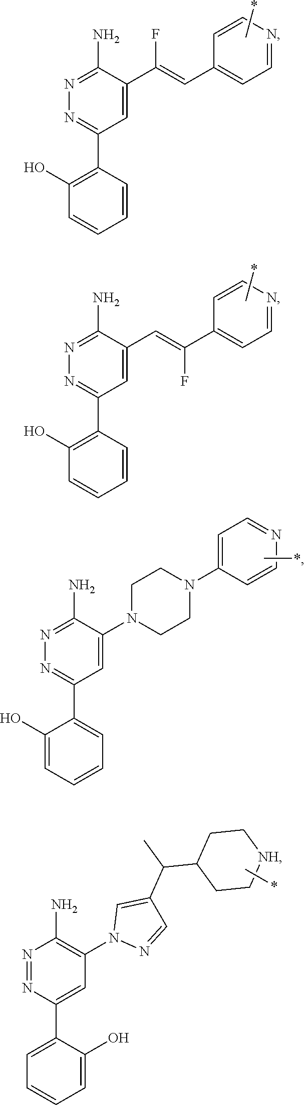

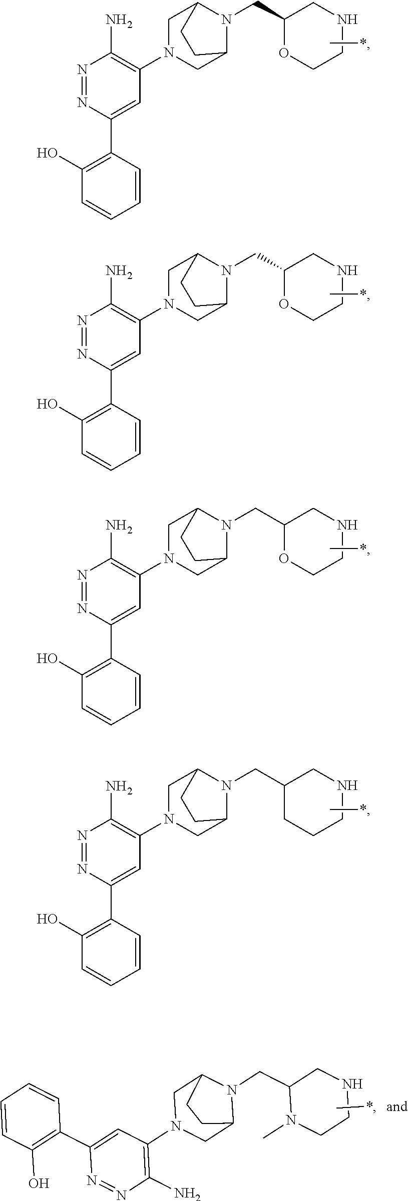

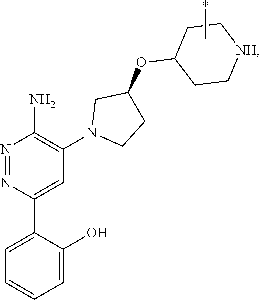

wherein [symbol] is the attachment point to the, linker, ULM group, and/or VLM group.

The compositions described herein exemplify some of the members of these types of small molecule target protein binding moieties. Such small molecule target protein binding moieties also include pharmaceutically acceptable salts, enantiomers, solvates, and polymorphs of these compositions, as well as other small molecules that may target a protein of interest. References which are cited herein below are incorporated by reference herein in their entirety.

### Therapeutic Compositions

Disclosed herein are pharmaceutical compositions comprising combinations of an effective amount of at least one bifunctional compound as described herein, in combination with a pharmaceutically effective amount of a carrier, additive or excipient, represents a further aspect of the present disclosure. In some embodiments, the pharmaceutical compositions disclosed herein further comprise an additional pharmaceutically active compound otherwise described herein.

The compositions present disclosure include, where applicable, the pharmaceutically acceptable salts, in particular acid or base addition salts, of compounds as described herein. Acids that can be used to prepare the pharmaceutically acceptable acid addition salts of the aforementioned base compounds and form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate, and pamoate [i.e., 1,1′-methylene-bis-(2-hydroxy-3 naphthoate)]salts, among others.

Bases that may be used to prepare pharmaceutically acceptable base salts of the present compounds that are acidic in nature are those that form non-toxic base salts with such compounds. Such non-toxic base salts include, but are not limited to those derived from such pharmacologically acceptable cations such as alkali metal cations (e.g., potassium and sodium) and alkaline earth metal cations (e.g., calcium, zinc and magnesium), ammonium or water-soluble amine addition salts such as N-methylglucamine-(meglumine), and the lower alkanolammonium and other base salts of pharmaceutically acceptable organic amines, among others.

The compounds as described herein may, in accordance with the disclosure, be administered in single or divided doses by the oral, parenteral or topical routes. Administration of the active compound may range from continuous (intravenous drip) to several oral administrations per day (for example, Q.I.D.) and may include oral, topical, parenteral, intramuscular, intravenous, sub-cutaneous, transdermal (which may include a penetration enhancement agent), buccal, sublingual and suppository administration, among other routes of administration. Enteric coated oral tablets may also be used to enhance bioavailability of the compounds from an oral route of administration. The most effective dosage form will depend upon the pharmacokinetics of the particular agent chosen as well as the severity of disease in the patient. Administration of compounds according to the present disclosure as sprays, mists, or aerosols for intra-nasal, intra-tracheal or pulmonary administration may also be used. The present disclosure therefore also is directed to pharmaceutical compositions comprising an effective amount of compound as described herein, optionally in combination with a pharmaceutically acceptable carrier, additive or excipient. Compounds according to the present disclosure may be administered in immediate release, intermediate release or sustained or controlled release forms. Sustained or controlled release forms are preferably administered orally, but also in suppository and transdermal or other topical forms. Intramuscular injections in liposomal form may also be used to control or sustain the release of compound at an injection site.

The compositions as described herein may be formulated in a conventional manner using one or more pharmaceutically acceptable carriers and may also be administered in controlled-release formulations. Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions as described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Sterile injectable forms of the compositions as described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as Ph. Helv or similar alcohol.

The pharmaceutical compositions as described herein may be orally administered in any orally acceptable dosage form including, but not limited to, capsules, tablets, aqueous suspensions or solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, the pharmaceutical compositions as described herein may be administered in the form of suppositories for rectal administration. These can be prepared by mixing the agent with a suitable non-irritating excipient, which is solid at room temperature but liquid at rectal temperature and therefore will melt in the rectum to release the drug. Such materials include cocoa butter, beeswax and polyethylene glycols.

The amount of compound in a pharmaceutical composition as described herein that may be combined with the carrier materials to produce a single dosage form will vary depending upon the host and disease treated and the particular mode of administration. Preferably, the compositions should be formulated to contain between about 0.05 milligram to about 750 milligrams or more, more preferably about 1 milligram to about 600 milligrams, and even more preferably about 10 milligrams to about 500 milligrams of active ingredient, alone or in combination with at least one other compound according to the present disclosure.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

A patient or subject in need of therapy using compounds according to the methods described herein can be treated by administering to the patient (subject) an effective amount of the compound according to the present disclosure including pharmaceutically acceptable salts, solvates or polymorphs, thereof optionally in a pharmaceutically acceptable carrier or diluent, either alone, or in combination with other known therapeutic agents as otherwise identified herein.

These compounds can be administered by any appropriate route, for example, orally, parenterally, intravenously, intradermally, or subcutaneously.

The active compound is included in the pharmaceutically acceptable carrier or diluent in an amount sufficient to deliver to a patient a therapeutically effective amount for the desired indication, without causing serious toxic effects in the patient treated. A preferred dose of the active compound for all of the herein-mentioned conditions is in the range from about 10 ng/kg to 300 mg/kg, preferably 0.1 to 100 mg/kg per day, more generally 0.5 to about 25 mg per kilogram body weight of the recipient/patient per day.

The compound is conveniently administered in any suitable unit dosage form, including but not limited to one containing less than 1 mg, 1 mg to 3000 mg, preferably 5 to 500 mg of active ingredient per unit dosage form. An oral dosage of about 25-250 mg is often convenient.

The active ingredient is preferably administered to achieve peak plasma concentrations of the active compound of about 0.00001-30 mM, preferably about 0.1-30 μM. This may be achieved, for example, by the intravenous injection of a solution or formulation of the active ingredient, optionally in saline, or an aqueous medium or administered as a bolus of the active ingredient. Oral administration is also appropriate to generate effective plasma concentrations of active agent.

The concentration of active compound in the drug composition will depend on absorption, distribution, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

Oral compositions will generally include an inert diluent or an edible carrier. They may be enclosed in gelatin capsules or compressed into tablets. For the purpose of oral therapeutic administration, the active compound or its prodrug derivative can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid, Primogel, or corn starch; a lubricant such as magnesium stearate or Sterotes; a glidants such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or enteric agents.

The active compound or pharmaceutically acceptable salt thereof can be administered as a component of an elixir, suspension, syrup, wafer, chewing gum or the like. A syrup may contain, in addition to the active compounds, sucrose as a sweetening agent and certain preservatives, dyes and colorings and flavors.

The active compound or pharmaceutically acceptable salts thereof can also be mixed with other active materials that do not impair the desired action, or with materials that supplement the desired action, such as anti-cancer agents, including pembrolizumab, among others. In certain preferred aspects of the disclosure, one or more compounds according to the present disclosure are coadministered with another bioactive agent, such as an anti-cancer agent or a would healing agent, including an antibiotic, as otherwise described herein.

Solutions or suspensions used for parenteral, intradermal, subcutaneous, application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

If administered intravenously, preferred carriers are physiological saline or phosphate buffered saline (PBS).

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

Liposomal suspensions may also be pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound are then introduced into the container. The container is then swirled by hand to free lipid material from the sides of the container and to disperse lipid aggregates, thereby forming the liposomal suspension.

### Therapeutic Methods

In an additional aspect, the description provides therapeutic compositions comprising an effective amount of a compound as described herein or salt form thereof, and a pharmaceutically acceptable carrier. The therapeutic compositions modulate protein degradation in a patient or subject, for example, an animal such as a human, and can be used for treating or ameliorating disease states or conditions that are modulated through the degraded protein.

The terms “treat,” “treating,” and “treatment,” etc., as used herein, refer to any action providing a benefit to a patient for which the present compounds may be administered, including the treatment of any disease state or condition which is modulated through the protein to which the present compounds bind. Disease states or conditions, including cancer such as lung cancer, including non-small cell lung cancer, which may be treated using compounds according to the present disclosure are set forth hereinabove.

The description provides therapeutic compositions as described herein for effectuating the degradation of proteins of interest for the treatment or amelioration of a disease, e.g., cancer. In certain additional embodiments, the disease is multiple myeloma. As such, in another aspect, the description provides a method of ubiquitinating/degrading a target protein in a cell. In certain embodiments, the method comprises administering a bifunctional compound as described herein comprising, e.g., a ULM and a PTM, preferably linked through a linker moiety, as otherwise described herein, wherein the ULM is coupled to the PTM and wherein the ULM recognizes a ubiquitin pathway protein (e.g., an ubiquitin ligase, such as a VHL E3 ubiquitin ligase) and the PTM recognizes the target protein such that degradation of the target protein will occur when the target protein is placed in proximity to the ubiquitin ligase, thus resulting in degradation/inhibition of the effects of the target protein and the control of protein levels. The control of protein levels afforded by the present disclosure provides treatment of a disease state or condition, which is modulated through the target protein by lowering the level of that protein in the cell, e.g., cell of a patient. In certain embodiments, the method comprises administering an effective amount of a compound as described herein, optionally including a pharmaceutically acceptable excipient, carrier, adjuvant, another bioactive agent or combination thereof.

In additional embodiments, the description provides methods for treating or ameliorating a disease, disorder or symptom thereof in a subject or a patient, e.g., an animal such as a human, comprising administering to a subject in need thereof a composition comprising an effective amount, e.g., a therapeutically effective amount, of a compound as described herein or salt form thereof, and a pharmaceutically acceptable excipient, carrier, adjuvant, another bioactive agent or combination thereof, wherein the composition is effective for treating or ameliorating the disease or disorder or symptom thereof in the subject.

In another aspect, the description provides methods for identifying the effects of the degradation of proteins of interest in a biological system using compounds according to the present disclosure.

In another embodiment, the present disclosure is directed to a method of treating a human patient in need for a disease state or condition modulated through a protein where the degradation of that protein will produce a therapeutic effect in the patient, the method comprising administering to a patient in need an effective amount of a compound according to the present disclosure, optionally in combination with another bioactive agent. The disease state or condition may be a disease caused by a microbial agent or other exogenous agent such as a virus, bacteria, fungus, protozoa or other microbe or may be a disease state, which is caused by overexpression of a protein, which leads to a disease state and/or condition

The term “disease state or condition” is used to describe any disease state or condition wherein protein dysregulation (i.e., the amount of protein expressed in a patient is elevated) occurs and where degradation of one or more proteins in a patient may provide beneficial therapy or relief of symptoms to a patient in need thereof. In certain instances, the disease state or condition may be cured.

Exemplary disease states or conditions which may be treated using the disclosed bifunctional compounds include asthma, autoimmune diseases such as multiple sclerosis, cancer, ciliopathies, cleft palate, diabetes, heart disease, hypertension, inflammatory bowel disease, mental retardation, mood disorder, obesity, refractive error, infertility, Angelman syndrome, Canavan disease, Coeliac disease, Charcot-Marie-Tooth disease, Cystic fibrosis, Duchenne muscular dystrophy, Haemochromatosis, Haemophilia, Klinefelter's syndrome, Neurofibromatosis, Phenylketonuria, Polycystic kidney disease, (PKD1) or 4 (PKD2) Prader-Willi syndrome, Sickle-cell disease, Tay-Sachs disease, and Turner syndrome. In certain embodiments, the compounds disclosed herein are used to treat cancer.

The term “neoplasia” or “cancer” is used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated. As used herein, the term neoplasia is used to describe all cancerous disease states and embraces or encompasses the pathological process associated with malignant hematogenous, ascitic and solid tumors. Exemplary cancers which may be treated by the present compounds either alone or in combination with at least one additional anti-cancer agent include squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas. Additional cancers which may be treated using compounds according to the present disclosure include, for example, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL and Philadelphia chromosome positive CML.

The term “bioactive agent” is used to describe an agent, other than a compound according to the present disclosure, which is used in combination with the present compounds as an agent with biological activity to assist in effecting an intended therapy, inhibition and/or prevention/prophylaxis for which the present compounds are used. Preferred bioactive agents for use herein include those agents which have pharmacological activity similar to that for which the present compounds are used or administered and include for example, anti-cancer agents, antiviral agents, especially including anti-HIV agents and anti-HCV agents, antimicrobial agents, antifungal agents, etc.

The term “additional anti-cancer agent” is used to describe an anti-cancer agent, which may be combined with compounds according to the present disclosure to treat cancer. These agents include, for example, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhbitor, a c-MET inhibitor, a PARP inhibitor, a Cdk inhibitor, an EGFR TK inhibitor, an IGFR-TK inhibitor, an anti-HGF antibody, a PI3 kinase inhibitor, an AKT inhibitor, an mTORC1/2 inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258); 3-[5-(methylsulfonylpiperadinemethyl)-indolyl-quinolone, vatalanib, AG-013736, AVE-0005, goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, adriamycin, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gleevec, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O—(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonist, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa and mixtures thereof.

The term “pharmaceutically acceptable salt” is used throughout the specification to describe, where applicable, a salt form of one or more of the compounds described herein which are presented to increase the solubility of the compound in the gastic juices of the patient's gastrointestinal tract in order to promote dissolution and the bioavailability of the compounds. Pharmaceutically acceptable salts include those derived from pharmaceutically acceptable inorganic or organic bases and acids, where applicable. Suitable salts include those derived from alkali metals such as potassium and sodium, alkaline earth metals such as calcium, magnesium and ammonium salts, among numerous other acids and bases well known in the pharmaceutical art. Sodium and potassium salts are particularly preferred as neutralization salts of the phosphates according to the present disclosure.

The term “pharmaceutically acceptable derivative” is used throughout the specification to describe any pharmaceutically acceptable prodrug form (such as an ester, amide other prodrug group), which, upon administration to a patient, provides directly or indirectly the present compound or an active metabolite of the present compound.

### EXEMPLIFICATION

### Abbreviations

ACN: acetonitrile
ADDP: 1,1′-(azodicarbonyl)dipiperidine
BAST: N,N-bis(2-methoxyethyl)aminosulfur trifluoride
BPO: benzoyl peroxide
Cbz: Carbonylbezyloxy
DAST: diethylaminosulfur trifluoride
DBE: 1,2-dibromoethane
DCM: dichloromethane
DEAD: diethyl azodicarboxylate
DIAD: diisopropyl azodicarboxylate
DIBAL: disiobutylaluminium hydride
DIEA or DIPEA: diisopropylethylamine
DMA: N,N-dimethylacetamide
DMF: N,N-dimethylformamide
DMP: Dess-Martin periodinane
EA: ethyl acetate
EDCI: 1-ethyl-3-(3-dimethylaminopropyl)carbodiimide
HBTU: N,N,N′N′-tetramethyl-O-(1H-benzotriazol-1-yl)uronium hexafluorophosphate
HMDS: bis9trimethylsilyl)amine
HMPA: hexamethylphosphoramide
LDA: lithium diisopropylamide
MCPBA: meta-chloroperoxybenzoic acid
MsCl: methanesulfonyl chloride
M.W: microwave
NBS: N-bromosuccinimide
NMP: N-methylpyrrolidone
PCC: pyridinium chlorochromate
Pd-118 or Pd(dtpf)Cl₂: 1,1′-bis(di-tert-butylphosphino)ferrocene dichloropalladium
Pd(dppf)Cl₂: 1,1′-bis(diphenylphosphino)ferrocene dichloropalladium
Pd(dba)₂: bis(dibenzylideneacetone)palladium
Pd₂(dba)₃: Tris(dibenzylideneacetone)dipalladium
PPTS: pyridium p-tolunesulfonate
PTSA: p-toluenesulfonic acid
RuPhos-Pd-G3: XPhos-Pd-G3: [(2-dicyclohexylphosphino-2′,6′-diisopropoxy-1,1′-biphenyl)-2-(2′-amino-1,1′-biphenyl)] palladium(II) methanesulfonate
RuPhos-Pd-G2: Chloro[(2-dicyclohexylphosphino-2′,6′-diisopropoxy-1,1′-biphenyl)-2-(2′-amino-1,1′-biphenyl)] palladium(II)
SFC: supercritical fluid chromatography
t-BuXPhos-Pd-G3: [(2-di-tert-butylphosphino-2′,4′,6′-triisopropyl-1,1′-biphenyl)-2-(2′-amino-1,1′-biphenyl)] palladium(II) methanesulfonate
TEA: trimethylamine
TFA: trifluoroacetic acid
TLC: thin layer chromatography
TMP: 2,2,6,6-tetramethylpiperidine
TEMPO: 2,2,6,6-tetramethylpiperidine-N-oxide
TosCl or TsCl: p-toluenesulfonyl chloride
TsOH: p-toluenesulfonic acid
XantPhos: 4,5-bis(diphenylphosphino)-9,9-dimethylxanthene
XPhos: 2-dicyclohexylphosphino-2′4′6′-triisopropylbiphenyl
XPhos-Pd-G3: [(2-dicyclohexylphosphino-2′,4′,6′-triisopropyl-1,1′-biphenyl)-2-(2′-amino-1,1′-biphenyl)] palladium(II) methanesulfonate
12354-85-7: bis(pentamethylcyclopentadienylrhodium dichloride)

### General Synthetic Approaches

The PTM represented by Formulas I thru VII can be synthesized by following the general synthetic routes detailed in the schemes below.

In the case of PTMs represented by Formula I, possible general synthetic approaches are outlined in the scheme below:

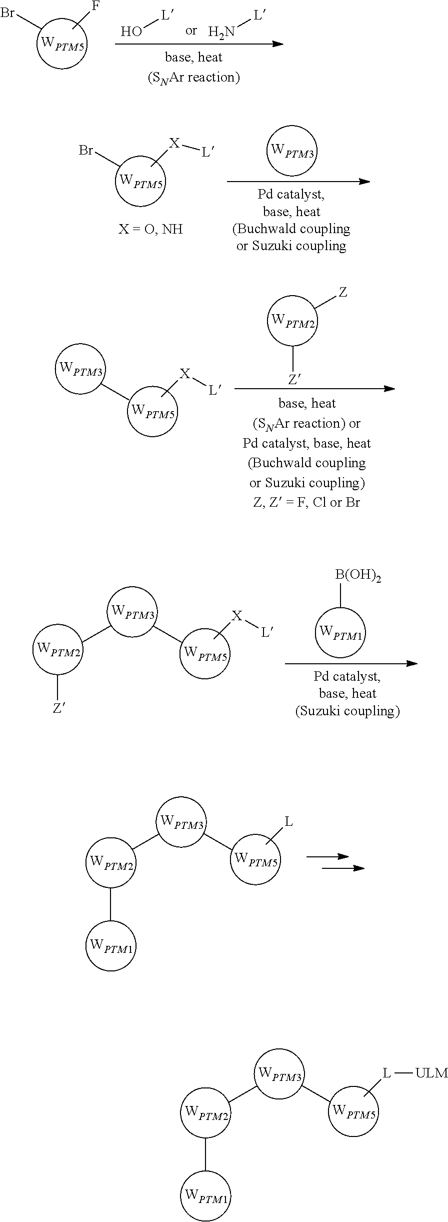

One skilled in the art would recognize that Buchwald coupling or S_{N}Ar approach in the scheme above is applicable when W_{PTM3} is a heterocycloalkyl connected to W_{PTM2} and W_{PTM5} through N atoms in W_{PTM3}. Otherwise, alternative transition metal-catalyzed coupling approaches (e.g., Suzuki coupling) would be more relevant (for example, if W_{PTM3} is an aryl or a heteroaryl).

One skilled in the art would also recognize that Buchwald coupling, or S_{N}Ar, or Suzuki coupling approach described in the scheme above would be mostly applicable when W_{PTM5} (or W_{PTM5A}) is an aryl or a heteroaryl. Otherwise, in cases where W_{PTM5} (or W_{PTM5A}) is a heterocycloalkyl the exact approach would depend on the nature of the functional group present in the above said heterocycloalkyl. Examples of possible approaches (reductive amination, nucleophilic substitution) are provided in the scheme below.

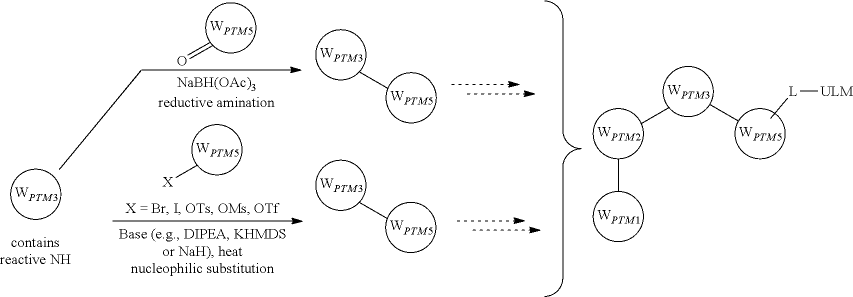

In the case where PTMs are represented by the general Formula V, and W_{PTM1} and W_{PTM2} are more specifically defined, the compounds can be synthesized as described in the scheme below. One skilled in the art would appreciate that alternative catalysts, temperature, solvent, and other experimental conditions compatible with S_{N}Ar reactions and transition metal-mediated coupling reactions can also be used.

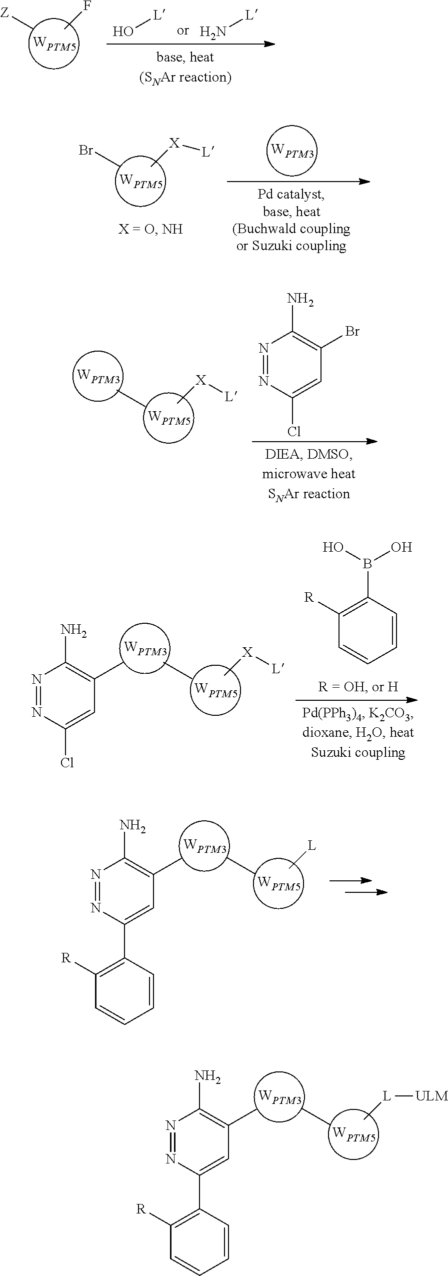

One skilled in the art would also appreciate that a modified approach can be utilized to enable attachment of PTMs via different chemical linkers. For example, in cases where W_{PTM5} is connected to L′ via a CH₂ group (X═CH₂ in the scheme above) one can envision an approach described in the scheme below:

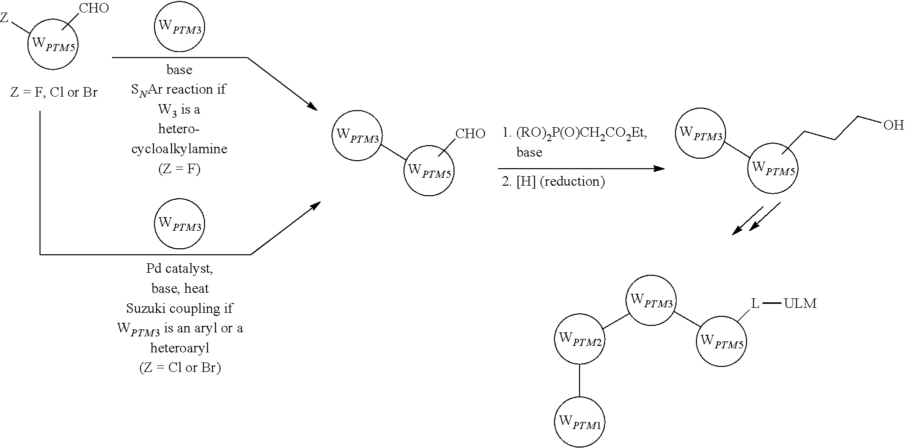

Alternatively, if W_{PTM5} is not present, PTM of an exemplary bifunctional degradative compound represented by Formula I can be synthesized according to the general scheme below if a reactive NH is present in W_{PTM3}. Alternatively, attaching linker to W_{PTM3} can be achieved using approaches described above for W_{PTM5}.

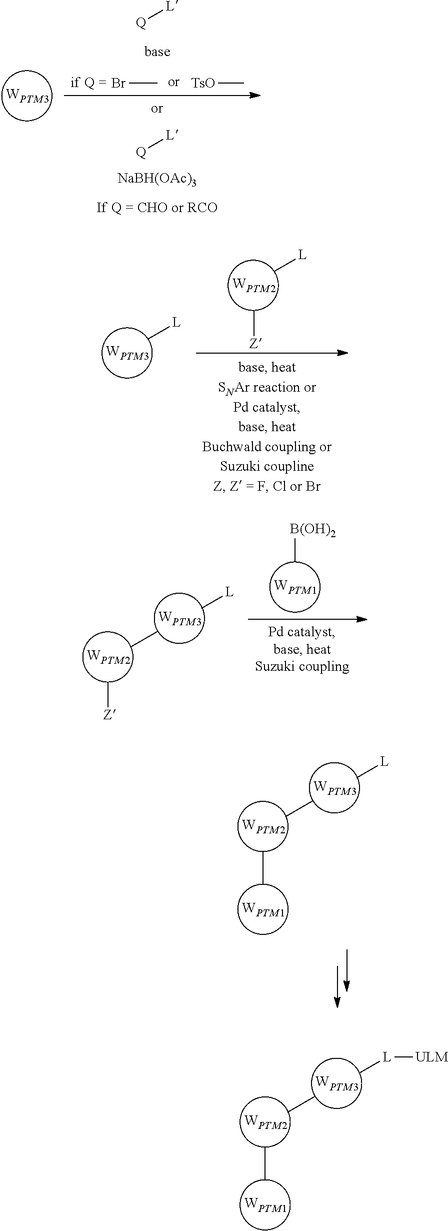

One skilled in the art will appreciate that synthetic approaches described herein can be modified to adapt to the specific nature of the W_{PTM1}, W_{PTM2}, W_{PTM3}, W_{PTM4}, W_{PTM5}, W_{PTM6} and W_{PTM7} rings. For example, in some embodiments, exemplary PTMs represented by Formula II can be prepared as described in the general synthetic scheme below where one skilled in the art would recognize that additional protection/deprotection steps may be required, depending upon the specific chemical nature of the compound:

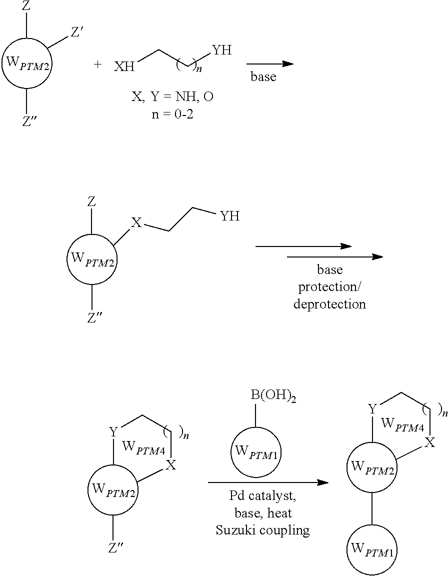

In some embodiments, where X represents NH, the bifunctional compound can be prepared according to one of the two schemes shown below, depending on whether W_{PTM5} is present, with W_{PTM5} or L becoming connected to the N atom of W_{PTM4}:

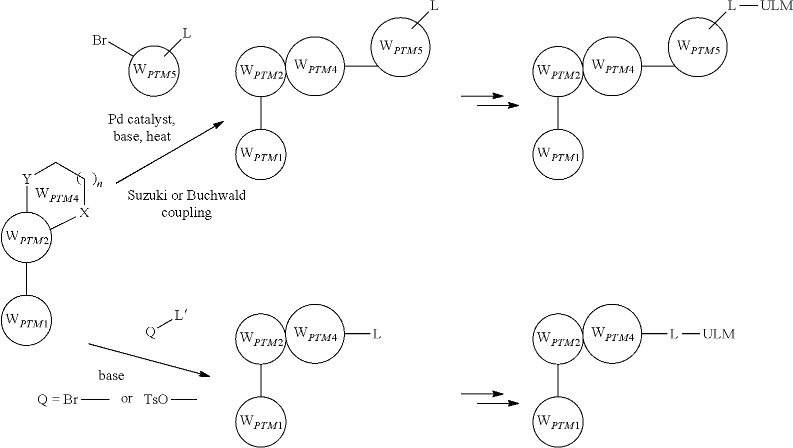

PTMs represented by general Formula IVa can be prepared according to the following general scheme:

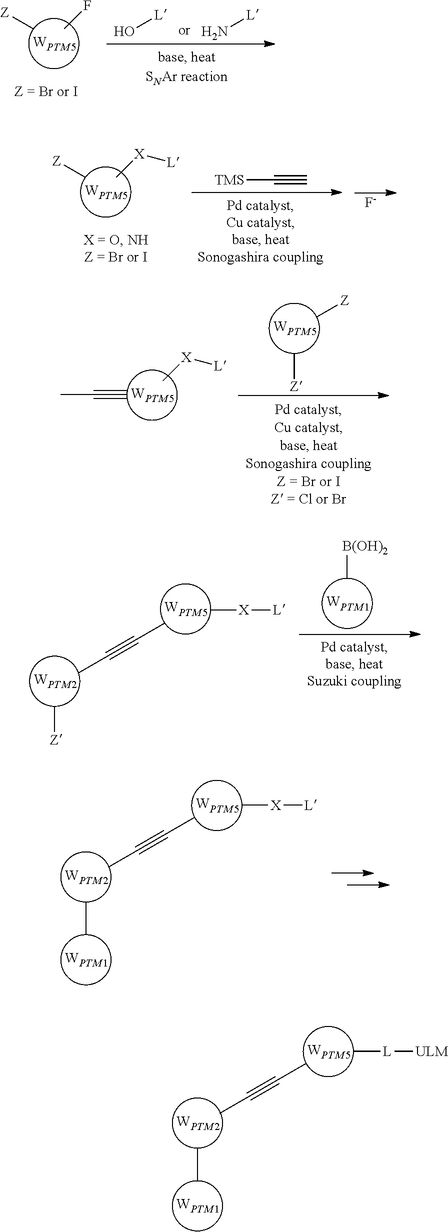

One skilled in the art will appreciate that the approach to the generation of W_{PTM5}-connected alkyne shown in the scheme above is most applicable when W_{PTM5} is an aryl or a heteroaryl. One skilled in the art will also appreciate that in cases where W_{PTM5} is a cycloalkyl, or a heterocycloalkyl, an alternative approach to the generation of the alkyne can be employed—for example, one based on the Ohira-Bestmann reagent shown in the scheme below.

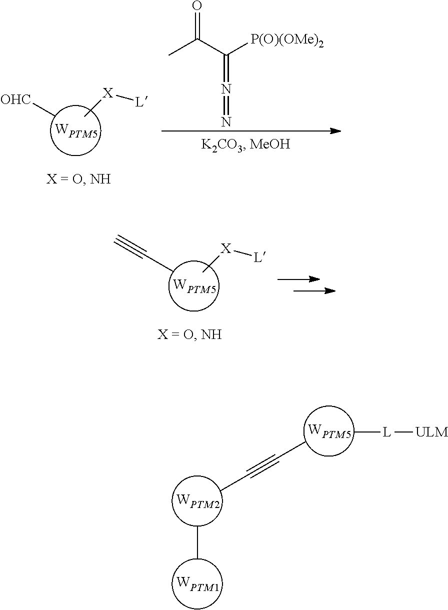

In addition, PTMs represented by general Formulas IVb1 and IVb2 can be prepared, in one possible approach, according to the following general scheme:

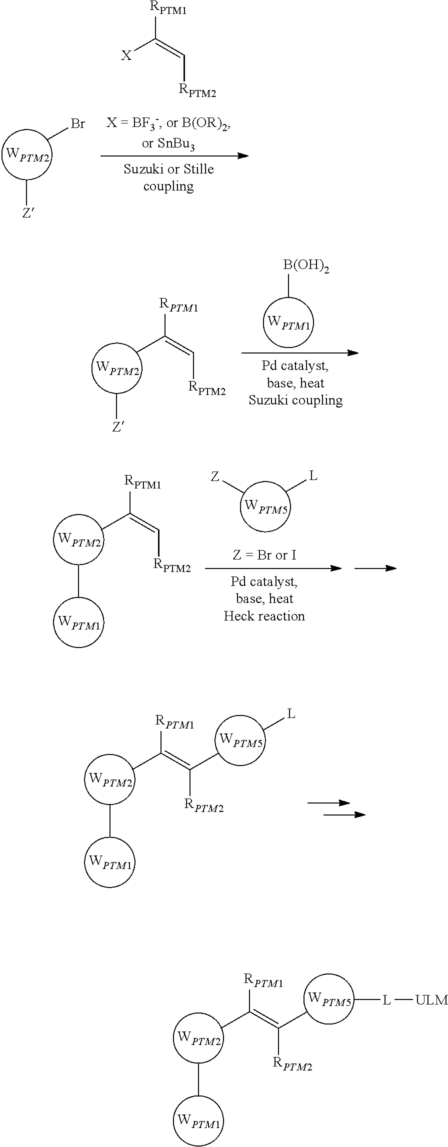

One skilled in the art will recognize that in many cases the applicable synthetic approaches may depend on the exact nature of the components comprising the PTM moiety as well as the nature of connectivity between them. For example, in cases where W_{PTM3} contains a reactive NH functionality a number of synthetic approaches relevant to the preparation of PTMs described by general formula VII can be envisoned, some nonlimiting examples of which are described in the scheme below:

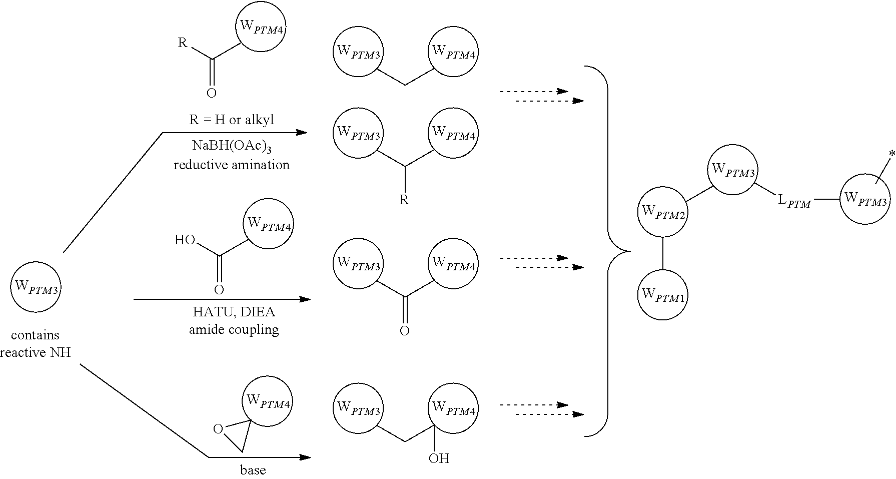

One skilled in the art will recognize that bifunctional compounds of the present disclosure can be prepared using various possible sequences of steps. In some embodiments, the bifunctional compounds of the present disclosure are assembled by joining together two fragments via a final connection in the middle of the linker using synthetic methods as shown in schemes below, for example, in the preferred general method A.

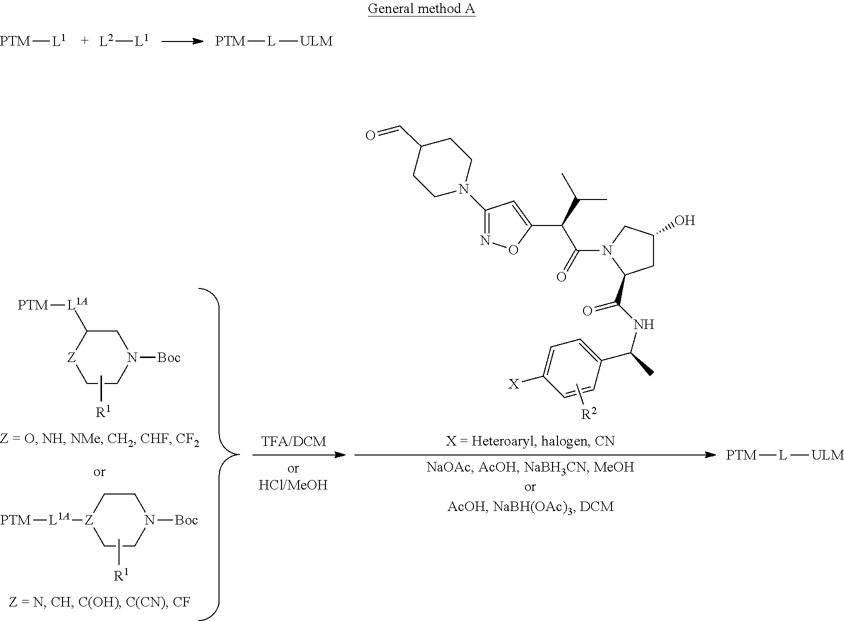

In some embodiments, the PTM-L^{1A}-heterocycloalkyl motif referenced in method A can be assembled by introducing a connection in L^{1A} as shown in general methods B and C.

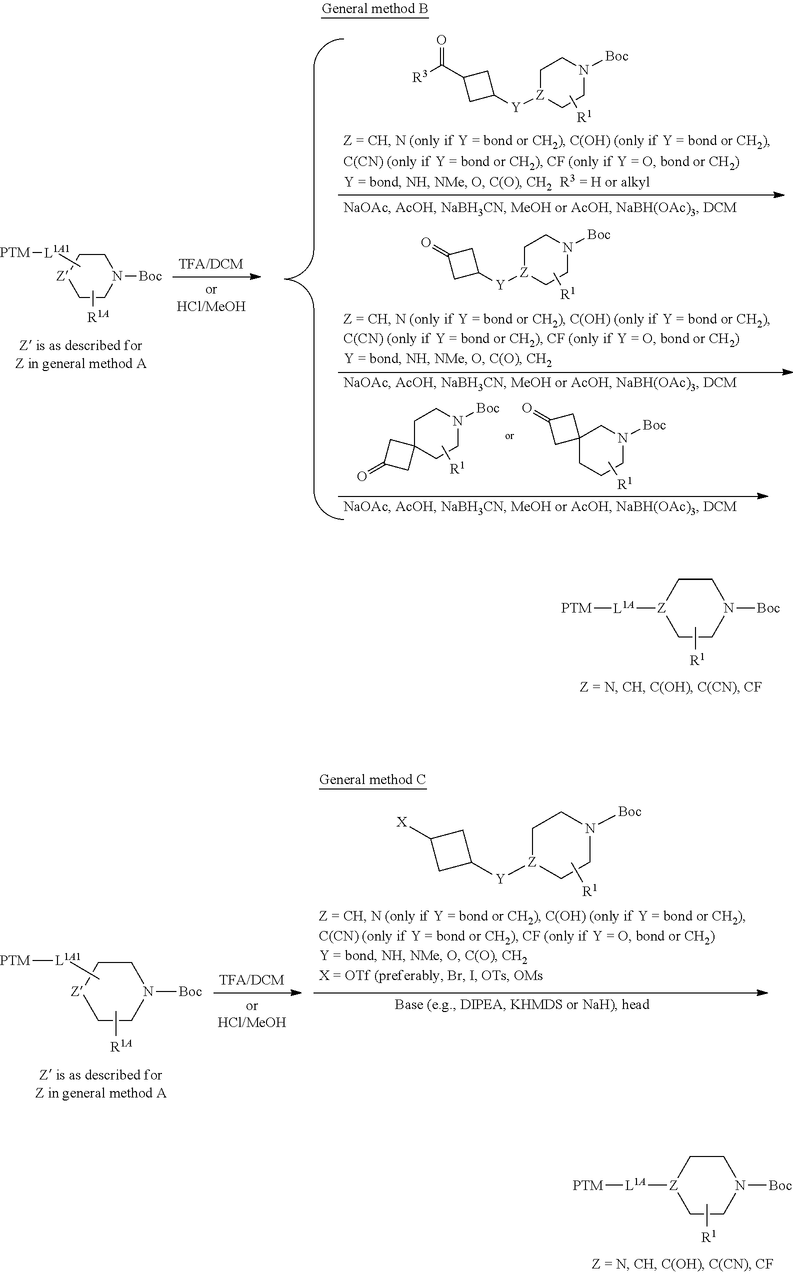

Those skilled in the art will appreciate that certain protecting groups can be used interchangeably in the context of general methods A, B and C. For example, Cbz protecting group may be used in place of Boc, in which case alternative methods of its cleavage (e.g., TMSI/ACN or Pd/C, H₂) may be employed.

Additionally, one skilled in the art will recognize that heterocycloalkyls in general methods A, B and C are also meant to include, by extension, heterocycloalkyls with ring sizes different from the ones explicitly shown, and that the nature of heterocycloalkyls is also meant to include, by extension, heteroaryls with reactive NH functionality. For example, in some embodiments, the above said heteroaryl can be an optionally substituted imidazole or an optionally substituted pyrazole. The above said heteroaryls can be subjected to the reductive amination or nucleophilic substitution conditions described in methods A, B, and C. One skilled in the art will also recognize that the above said heteroaryls may require different protecting groups (e.g., SEM) other than those shown in the scheme above.

In some embodiments the final connection can be introduced between the two parts of the ULM motif as shown in the scheme below for general method D.

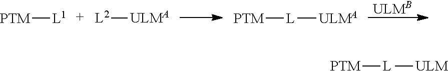

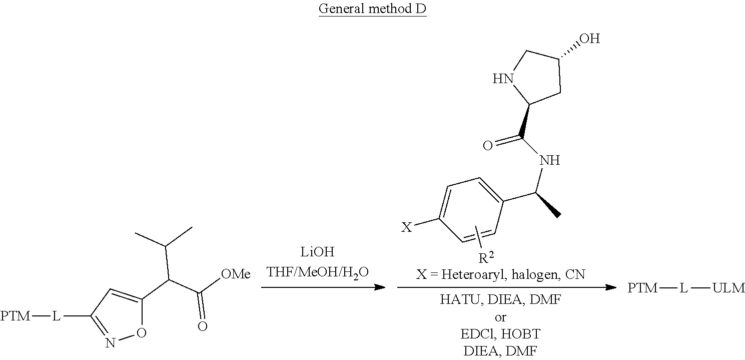

The synthetic realization and optimization of the bifunctional molecules as described herein may be approached in a step-wise or modular fashion.

In a very analogous way one can identify and optimize ligands for an E3 Ligase, i.e. ULMs/VLMs.

With PTMs and ULMs (e.g. VLMs) in hand, one skilled in the art can use known synthetic methods for their combination with or without a linker moiety. Linker moieties can be synthesized with a range of compositions, lengths and flexibility and functionalized such that the PTM and ULM groups can be attached sequentially to distal ends of the linker. Thus a library of bifunctional molecules can be realized and profiled in in vitro and in vivo pharmacological and ADMET/PK studies. As with the PTM and ULM groups, the final bifunctional molecules can be subject to iterative design and optimization cycles in order to identify molecules with desirable properties.

In some instances, protecting group strategies and/or functional group interconversions (FGIs) may be required to facilitate the preparation of the desired materials. Such chemical processes are well known to the synthetic organic chemist and many of these may be found in texts such as “Greene's Protective Groups in Organic Synthesis” Peter G. M. Wuts and Theodora W. Greene (Wiley), and “Organic Synthesis: The Disconnection Approach” Stuart Warren and Paul Wyatt (Wiley).

### Exemplary Synthesis of Compound 1

Prepared according to the scheme below using procedures commonly known to those skilled in the art.

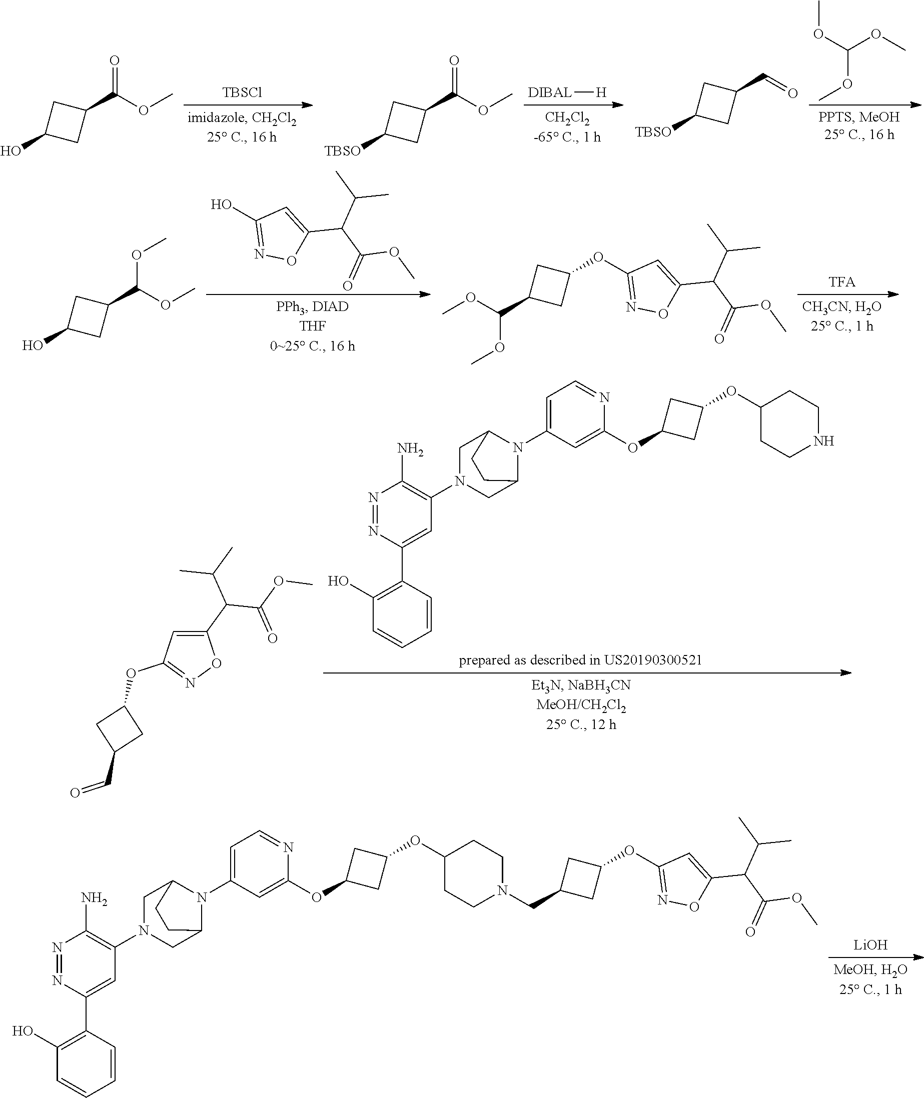

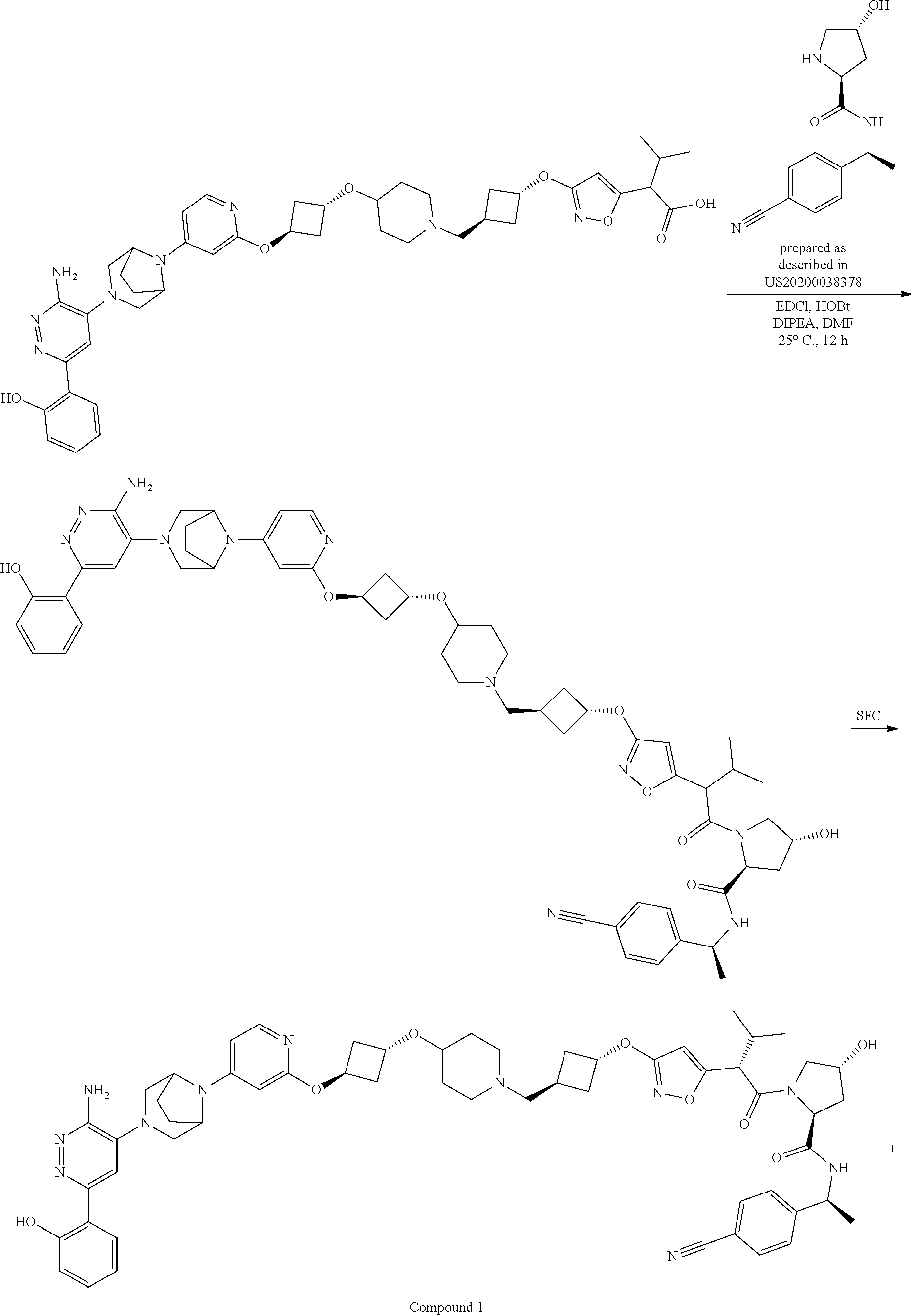

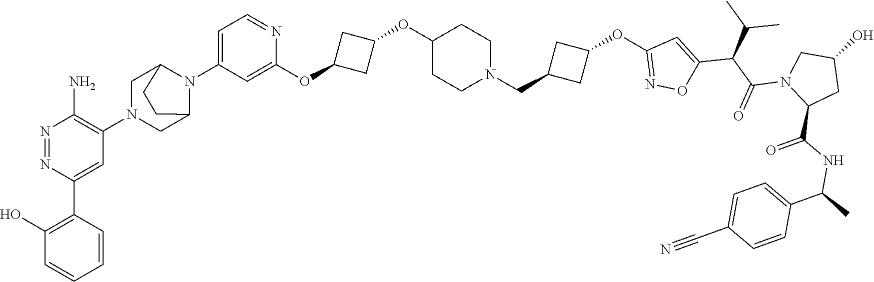 [SMILES: CC(C)[C@@H](C(=O)N1C[C@H](O)C[C@H]1C(=O)N[C@@H](C)c1ccc(C#N)cc1)c1cc(O[C@H]2C[C@H](CN3CCC(O[C@H]4C[C@H](Oc5cc(N6C7CCC6CN(c6cc(-c8ccccc8O)nnc6N)C7)ccn5)C4)CC3)C2)no1]

### Exemplary Synthesis of Compound 2

### Step 1

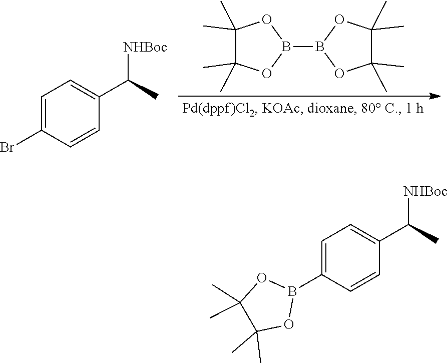

A flask was charged with tert-butyl N-[(1S)-1-(4-bromophenyl)ethyl]carbamate (1.4 g, 4.66 mmol, 1 eq), 4,4,5,5-tetramethyl-2-(4,4,5,5-tetramethyl-1,3,2-dioxaborolan-2-yl)-1,3,2-dioxaborolane (1.42 g, 5.60 mmol, 1.2 eq), (1,1′-bis(diphenylphosphino)ferrocene)palladium(II) dichloride (170 mg, 0.23 mmol, 0.05 eq), potassium acetate (915 mg, 9.33 mmol, 2 eq) and dioxane (30 mL). The mixture was purged with nitrogen for 10 minutes, then heated to 80° C. for 1 hour. The reaction mixture was cooled to 20° C. and filtered through a pad of celite. The filtrate was concentrated in vacuum. The crude product was purified on silica gel column (petroleum ether: ethyl acetate=10:1). Tert-butyl N-[(1S)-1-[4-(4,4,5,5-tetramethyl-1,3,2-dioxaborolan-2-yl)phenyl]ethyl]carbamate (1.9 g) was obtained as a colorless oil.

### Step 2

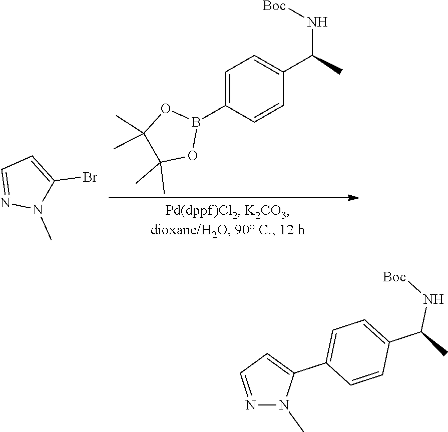

To a mixture of 5-bromo-1-methyl-pyrazole (800 mg, 4.97 mmol, 1 eq), potassium carbonate (1.37 g, 9.94 mmol, 2 eq) and tert-butyl N-(5-bromothiazol-4-yl)carbamate (2.07 g, 5.96 mmol, 1.2 eq) in water (5 mL) and dioxane (30 mL) was added [1,1′-bis(diphenylphosphino) ferrocene]dichloropalladium (II) (290 mg, 0.40 mmol, 0.08 eq) in one portion at 20° C. under nitrogen. The mixture was stirred at 90° C. for 12 hours. The mixture was cooled to 20° C. and poured into ice-water (w/w=1/1, 50 mL) and stirred for 10 minutes. The aqueous phase was extracted with ethyl acetate (40 mL×3). The combined organic phase was washed with brine (20 mL×2), dried with anhydrous sodium sulfate, filtered and concentrated in vacuum. The residue was purified by silica gel chromatography (petroleum ether/ethyl acetate=30/1 to 5/1) to afford tert-butyl N-[(1S)-1-[4-(2-methylpyrazol-3-yl)phenyl]ethyl]carbamate (1.6 g) as a yellow solid.

tert-Butyl N-[(1S)-1-[4-(2-methylpyrazol-3-yl)phenyl]ethyl]carbamate was converted to the title compound according to the scheme below using procedures commonly known to those skilled in the art.

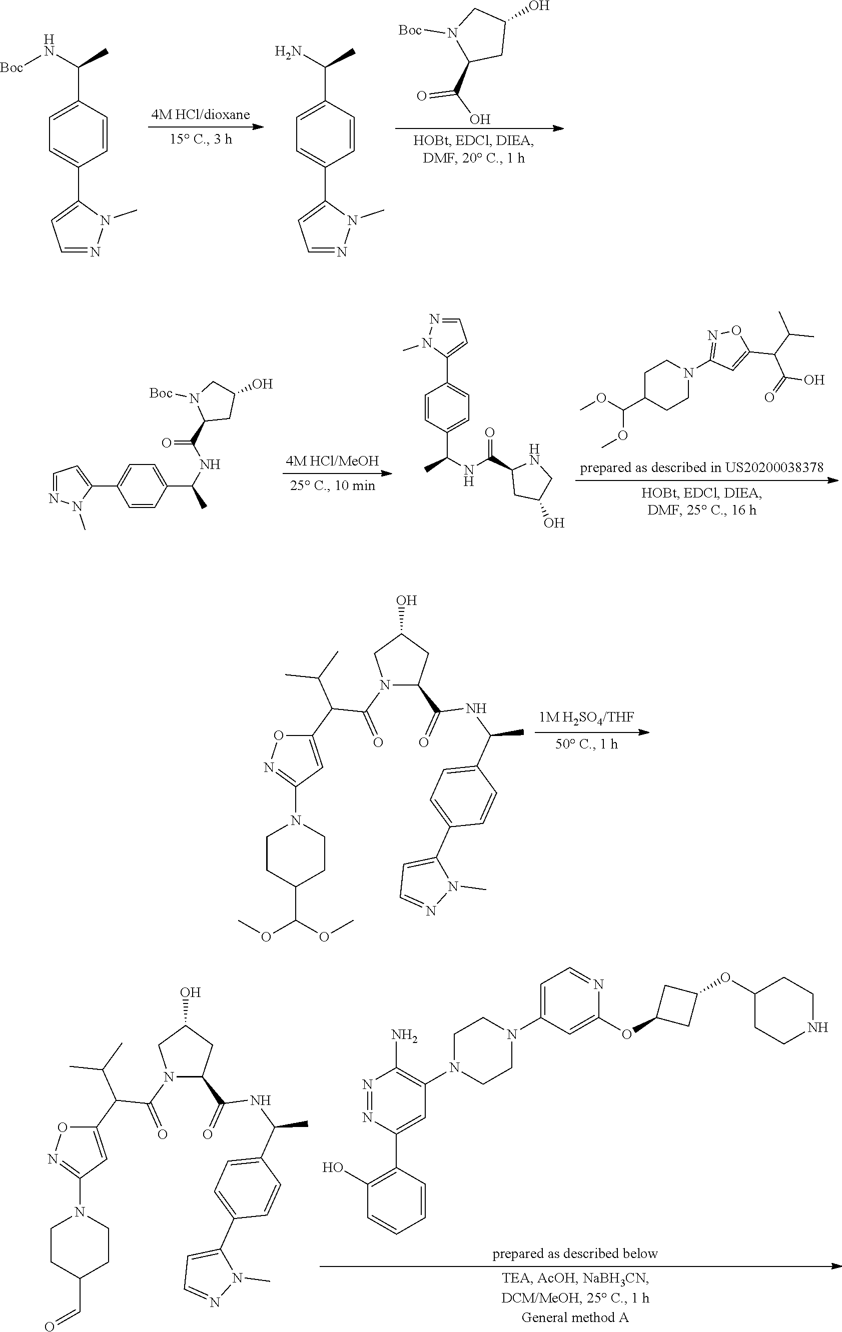

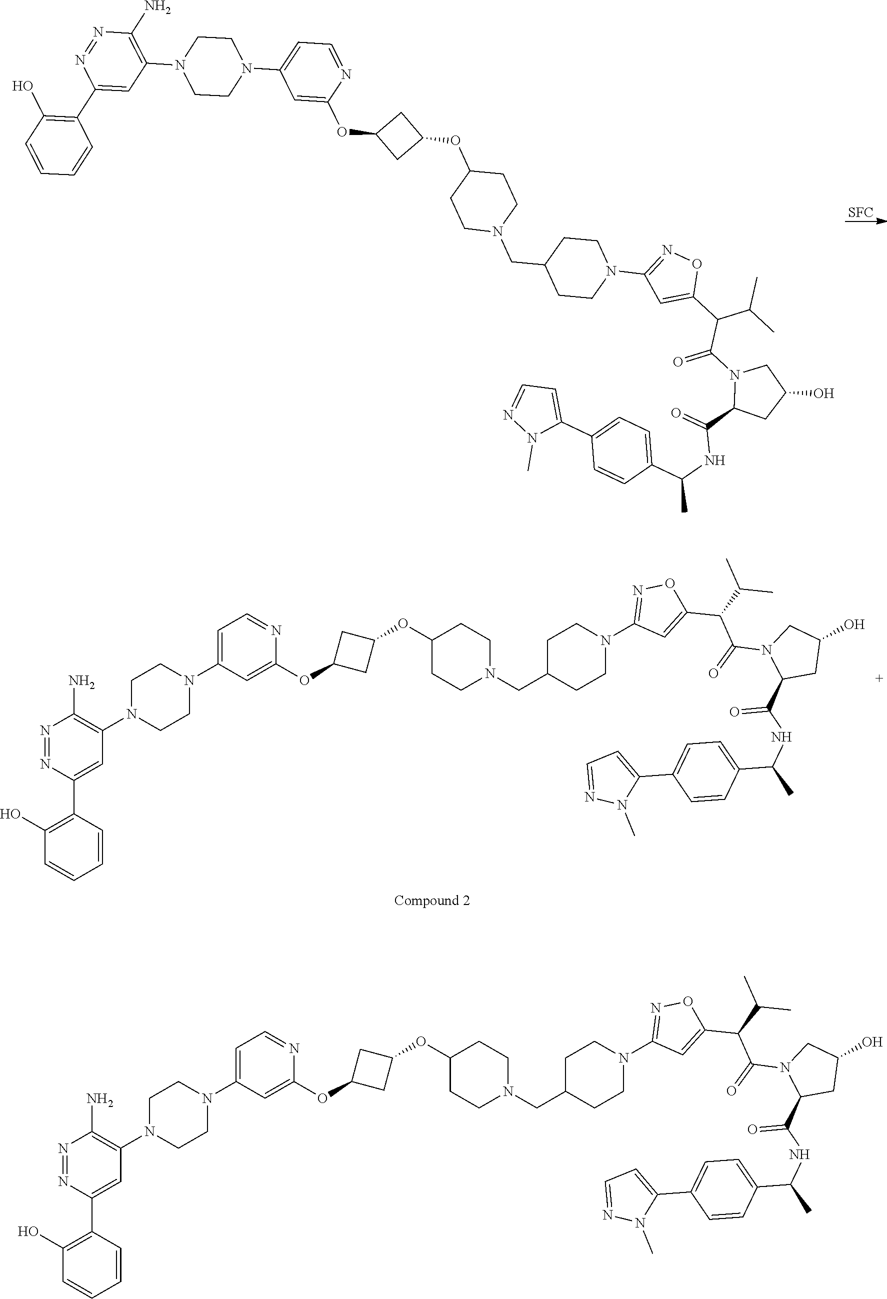 [SMILES: CC(C)C(C(=O)N1C[C@H](O)C[C@H]1C(=O)N[C@@H](C)c1ccc(-c2ccnn2C)cc1)c1cc(N2CCC(CN3CCC(O[C@H]4C[C@H](Oc5cc(N6CCN(c7cc(-c8ccccc8O)nnc7N)CC6)ccn5)C4)CC3)CC2)no1 ; "SFC" ; CC(C)[C@H](C(=O)N1C[C@H](O)C[C@H]1C(=O)N[C@@H](C)c1ccc(-c2ccnn2C)cc1)c1cc(N2CCC(CN3CCC(O[C@H]4C[C@H](Oc5cc(N6CCN(c7cc(-c8ccccc8O)nnc7N)CC6)ccn5)C4)CC3)CC2)no1 ; "+" ; CC(C)[C@@H](C(=O)N1C[C@H](O)C[C@H]1C(=O)N[C@@H](C)c1ccc(-c2ccnn2C)cc1)c1cc(N2CCC(CN3CCC(O[C@H]4C[C@H](Oc5cc(N6CCN(c7cc(-c8ccccc8O)nnc7N)CC6)ccn5)C4)CC3)CC2)no1]

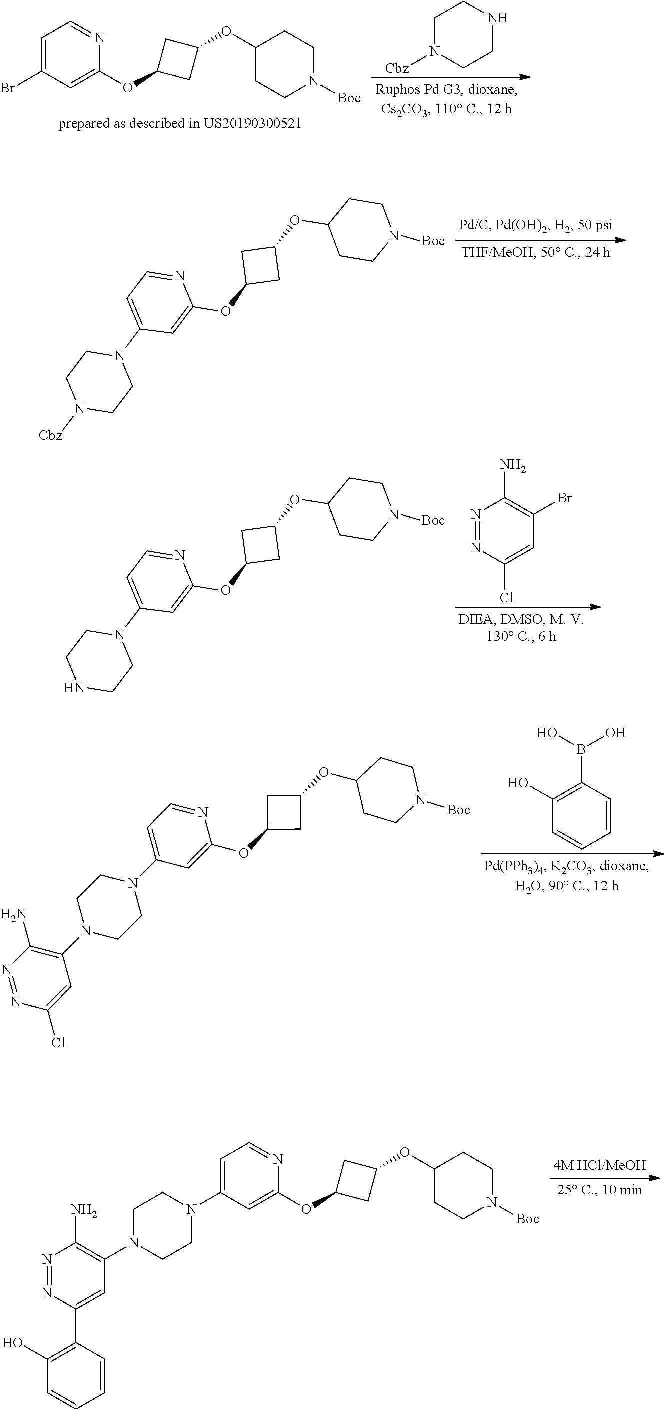

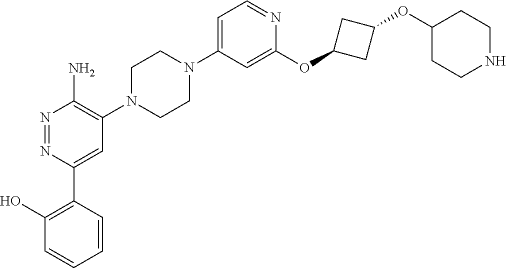 [SMILES: Nc1nnc(-c2ccccc2O)cc1N1CCN(c2ccnc(O[C@H]3C[C@H](OC4CCNCC4)C3)c2)CC1]

### Exemplary Synthesis of Compound 4

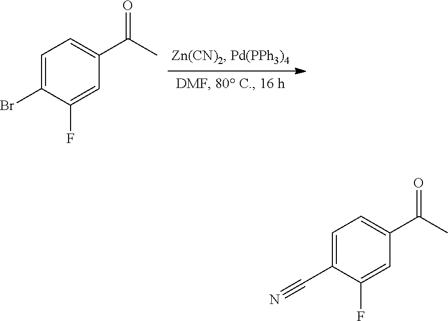

A mixture of 1-(4-bromo-3-fluoro-phenyl)ethanone (1 g, 4.61 mmol, 1 eq), zinc cyanide (1.08 g, 9.22 mmol, 2 eq) and tetrakis[triphenylphosphine]palladium(0) (532 mg, 0.46 mmol, 0.1 eq) in N,N-dimethylformamide (10 mL) was stirred at 80° C. for 16 hours under nitrogen. The reaction mixture was diluted with ethyl acetate (50 mL) and washed with water (10 mL×2) and brine (10 mL), dried over with anhydrous sodium sulfate, filtered and concentrated under reduced pressure to give a residue. The residue was purified by silica gel chromatography (petroleum ether/ethyl acetate=10/1). Compound 4-acetyl-2-fluoro-benzonitrile (530 mg, 3.25 mmol) was obtained as a light yellow solid.

### Step 2

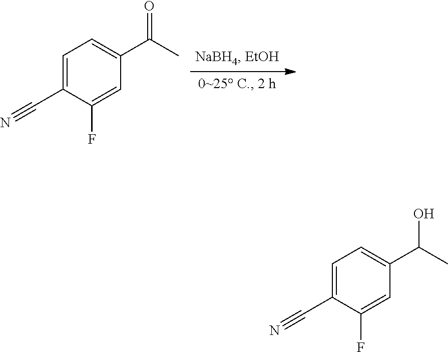

To a solution of 4-acetyl-2-fluoro-benzonitrile (0.26 g, 1.59 mmol, 1 eq) in ethanol (10 mL) was added sodium borohydride (121 mg, 3.19 mmol, 2 eq) at 0° C. The mixture was warmed to 25° C. and stirred for 2 hours. The reaction mixture was quenched by saturated ammonium chloride solution (0.1 mL) at 25° C., and then concentrated under reduced pressure to give a residue. The residue was purified by silica gel chromatography (petroleum ether/ethyl acetate=1/1). Compound 2-fluoro-4-(1-hydroxyethyl)benzonitrile (190 mg, 1.15 mmol) was obtained as a colorless oil.

### Step 3

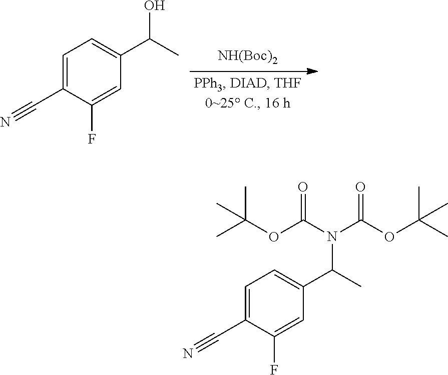 [SMILES: CC(O)c1ccc(C#N)c(F)c1 ; CC(c1ccc(C#N)c(F)c1)N(C(=O)OC(C)(C)C)C(=O)OC(C)(C)C ; "NH(Boc)2"]

To a mixture of tert-butyl N-tert-butoxycarbonylcarbamate (325 mg, 1.50 mmol, 1.3 eq), 2-fluoro-4-(1-hydroxyethyl)benzonitrile (190 mg, 1.15 mmol, 1 eq) and triphenylphosphine (453 mg, 1.73 mmol, 1.5 eq) in tetrahydrofuran (15 mL) was added diisopropyl azodicarboxylate (349 mg, 1.73 mmol, 1.5 eq) at 0° C. The mixture was warmed to 25° C. and stirred for 16 hours. The reaction mixture was concentrated under reduced pressure to give a residue. The residue was purified by silica gel chromatography (petroleum ether/ethyl acetate=40/1). Compound tert-butyl N-tert-butoxycarbonyl-N-[1-(4-cyano-3-fluoro-phenyl)ethyl]carbamate (70 mg, 0.19 mmol) was obtained as a colorless oil.

### Step 4

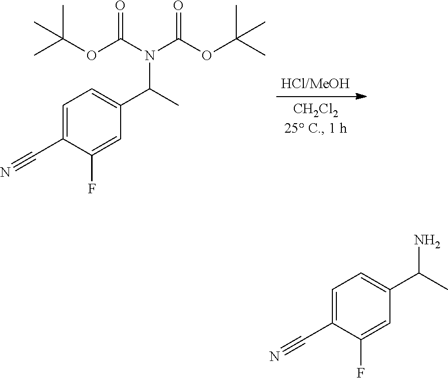

To a solution of tert-butyl N-tert-butoxycarbonyl-N-[1-(4-cyano-3-fluoro-phenyl)ethyl] carbamate (70 mg, 0.19 mmol, 1 eq) in dichloromethane (5 mL) was added hydrochloric acid/methanol (4 M, 1 mL, 20.82 eq) at 25° C., and the mixture was stirred for 1 hour. The reaction mixture was concentrated under reduced pressure to give a residue. Compound 4-(1-aminoethyl)-2-fluoro-benzonitrile (38.5 mg, crude, hydrochloride) was obtained as a white solid and used directly in the next step.

4-(1-aminoethyl)-2-fluorobenzonitrile was converted to the title compound as shown in schemes below using procedures commonly known to those skilled in the art.

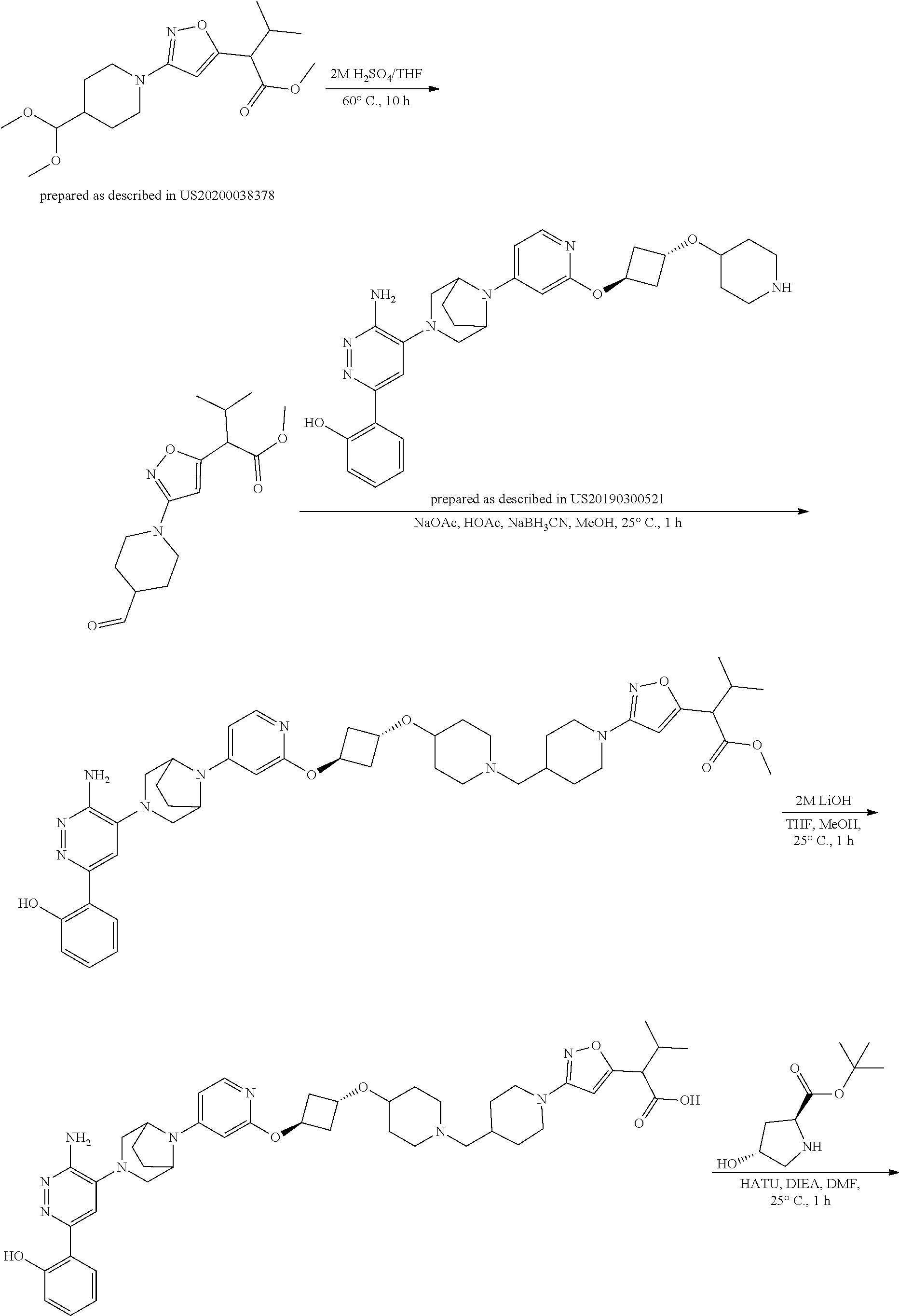

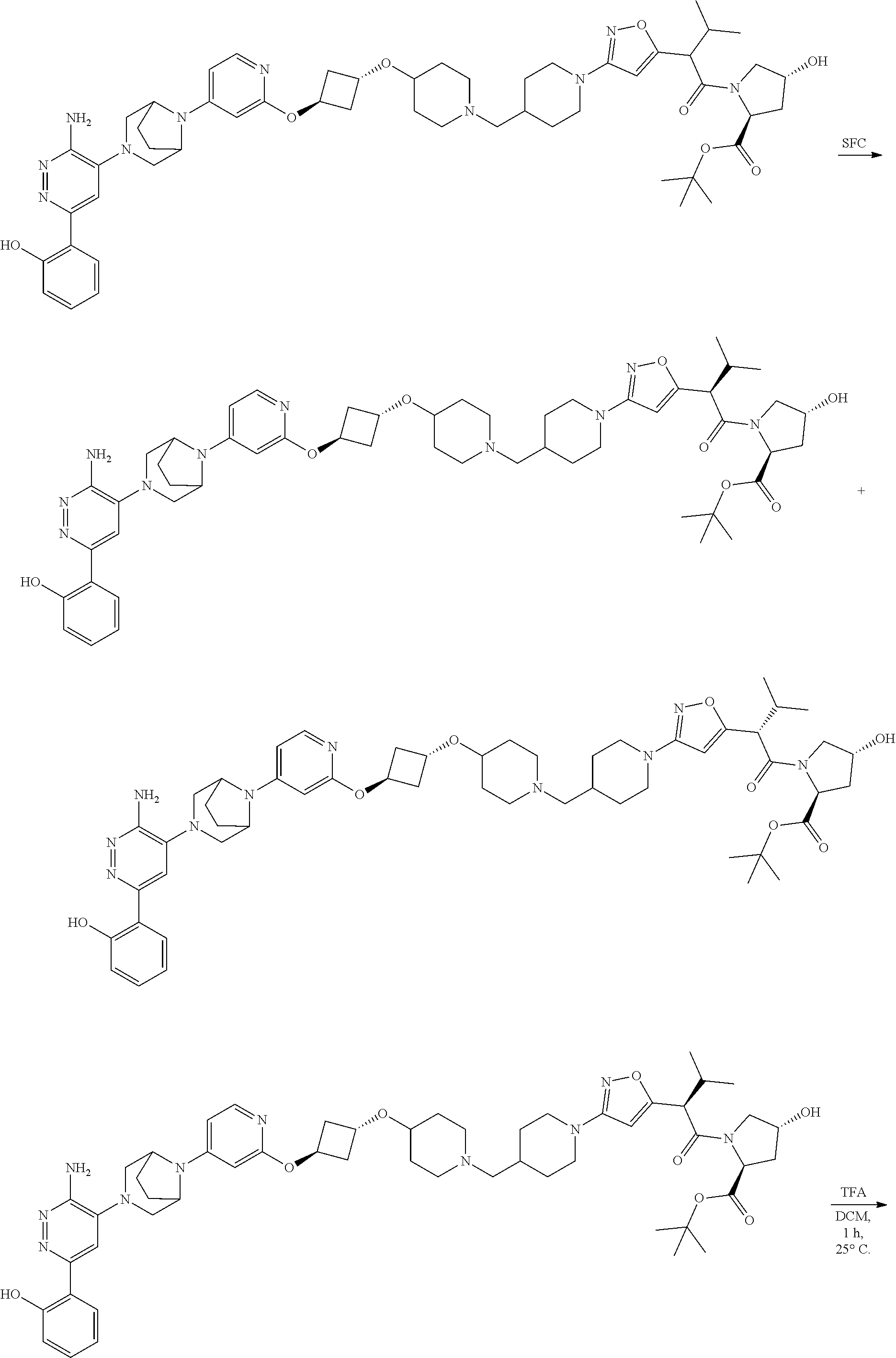

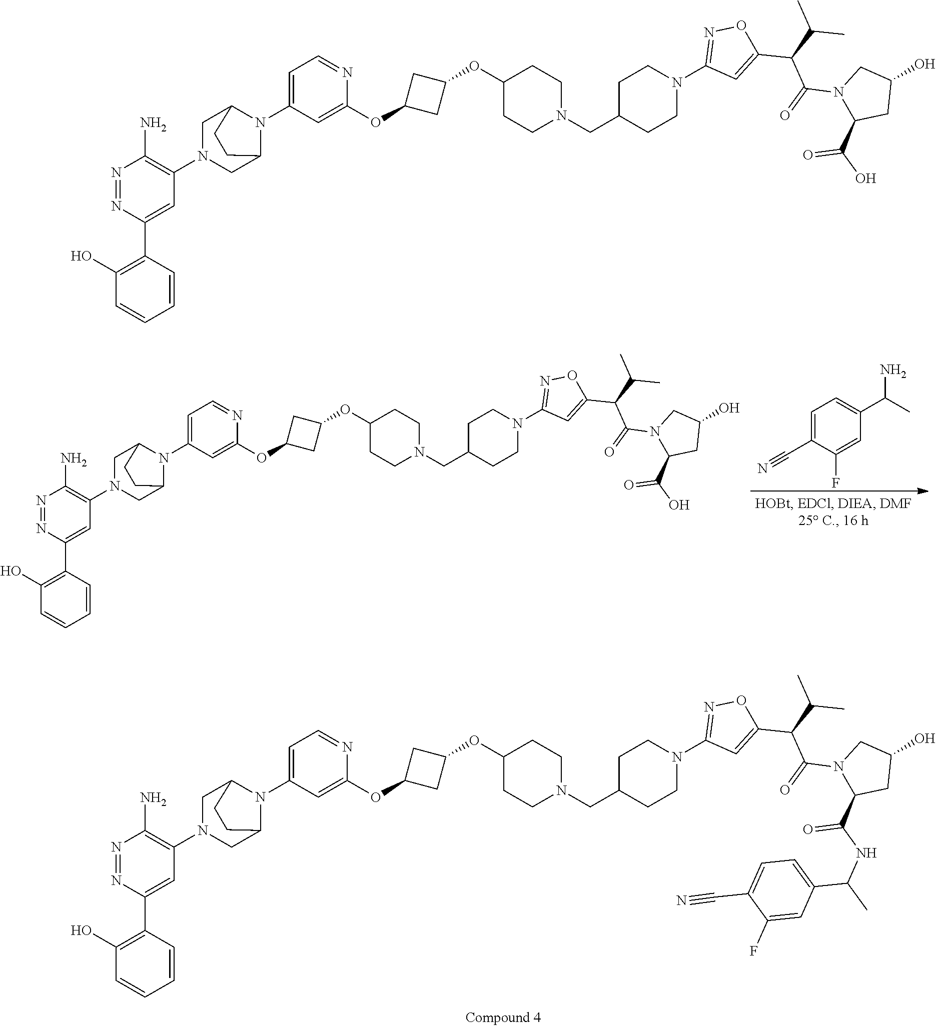 [SMILES: CC(C)[C@@H](C(=O)N1C[C@H](O)C[C@H]1C(=O)O)c1cc(N2CCC(CN3CCC(O[C@H]4C[C@H](Oc5cc(N6C7CCC6CN(c6cc(-c8ccccc8O)nnc6N)C7)ccn5)C4)CC3)CC2)no1 ; CC(C)[C@@H](C(=O)N1C[C@H](O)C[C@H]1C(=O)O)c1cc(N2CCC(CN3CCC(O[C@H]4C[C@H](Oc5cc(N6C7CCC6CN(c6cc(-c8ccccc8O)nnc6N)C7)ccn5)C4)CC3)CC2)no1 ; CC(N)c1ccc(C#N)c(F)c1 ; CC(NC(=O)[C@@H]1C[C@@H](O)CN1C(=O)[C@@H](c1cc(N2CCC(CN3CCC(O[C@H]4C[C@H](Oc5cc(N6C7CCC6CN(c6cc(-c8ccccc8O)nnc6N)C7)ccn5)C4)CC3)CC2)no1)C(C)C)c1ccc(C#N)c(F)c1]

Compounds 3 and 5 were prepared using procedures analogous to the Compound 4.

### Exemplary Synthesis of Compound 6, 7, 8, and 9

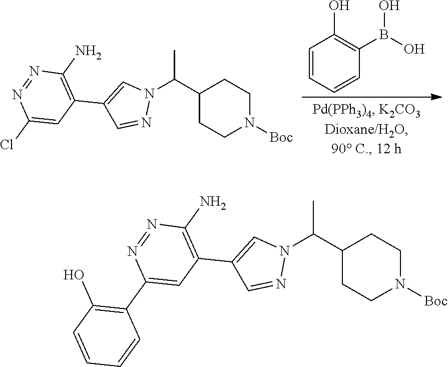 [SMILES: CC(C1CCN(C(=O)OC(C)(C)C)CC1)n1cc(-c2cc(Cl)nnc2N)cn1 ; OB(O)c1ccccc1O ; CC(C1CCN(C(=O)OC(C)(C)C)CC1)n1cc(-c2cc(-c3ccccc3O)nnc2N)cn1]

prepared as described in US20190300521

To a mixture of tert-butyl 4-[1-[4-(3-amino-6-chloro-pyridazin-4-yl)pyrazol-1-yl]ethyl] piperidine-1-carboxylate (3 g, 7.37 mmol, 1 eq), (2-hydroxyphenyl)boronic acid (1.53 g, 11.06 mmol, 1.5 eq) and potassium carbonate (3.06 g, 22.12 mmol, 3 eq) in dioxane (50 mL) and water (9 mL) was added tetrakis[triphenylphosphine]palladium(0) (852 mg, 0.73 mmol, 0.1 eq). The mixture was degassed and purged with nitrogen for 3 times. The reaction mixture was stirred at 90° C. for 12 hours. The reaction mixture was diluted with water (200 mL) and extracted with ethyl acetate (200 mL×2). The combined organic phase was washed with brine (50 mL), dried with anhydrous sodium sulfate, filtered and concentrated in vacuum. The residue was purified by prep-HPLC (column: Phenomenex luna C18 250*80 mm*10 um; mobile phase: [water (0.1% TFA)-ACN]; B %: 25%-50%, 20 min). Compound tert-butyl 4-[1-[4-[3-amino-6-(2-hydroxyphenyl)pyridazin-4-yl]pyrazol-1-yl]ethyl]piperidine-1-carboxylate (2.7 g, 5.81 mmol, 79% yield) was obtained as a yellow solid.

tert-Butyl 4-[1-[4-[3-amino-6-(2-hydroxyphenyl)pyridazin-4-yl]pyrazol-1-yl]ethyl]piperidine-1-carboxylate was converted to the title compounds as shown in schemes below using procedures commonly known to those skilled in the art.

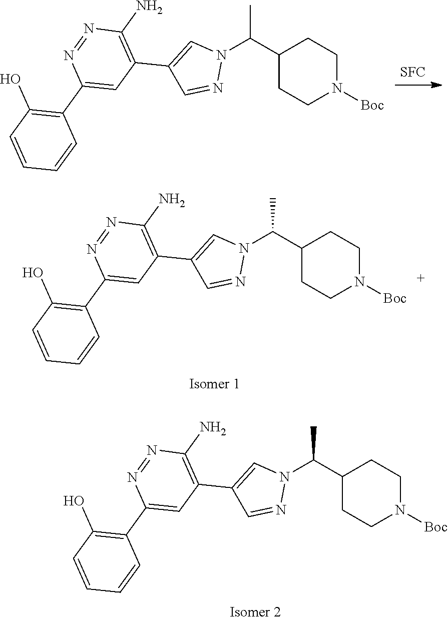

Absolute configuration arbitrarily assigned Absolute configuration arbitrarily assigned

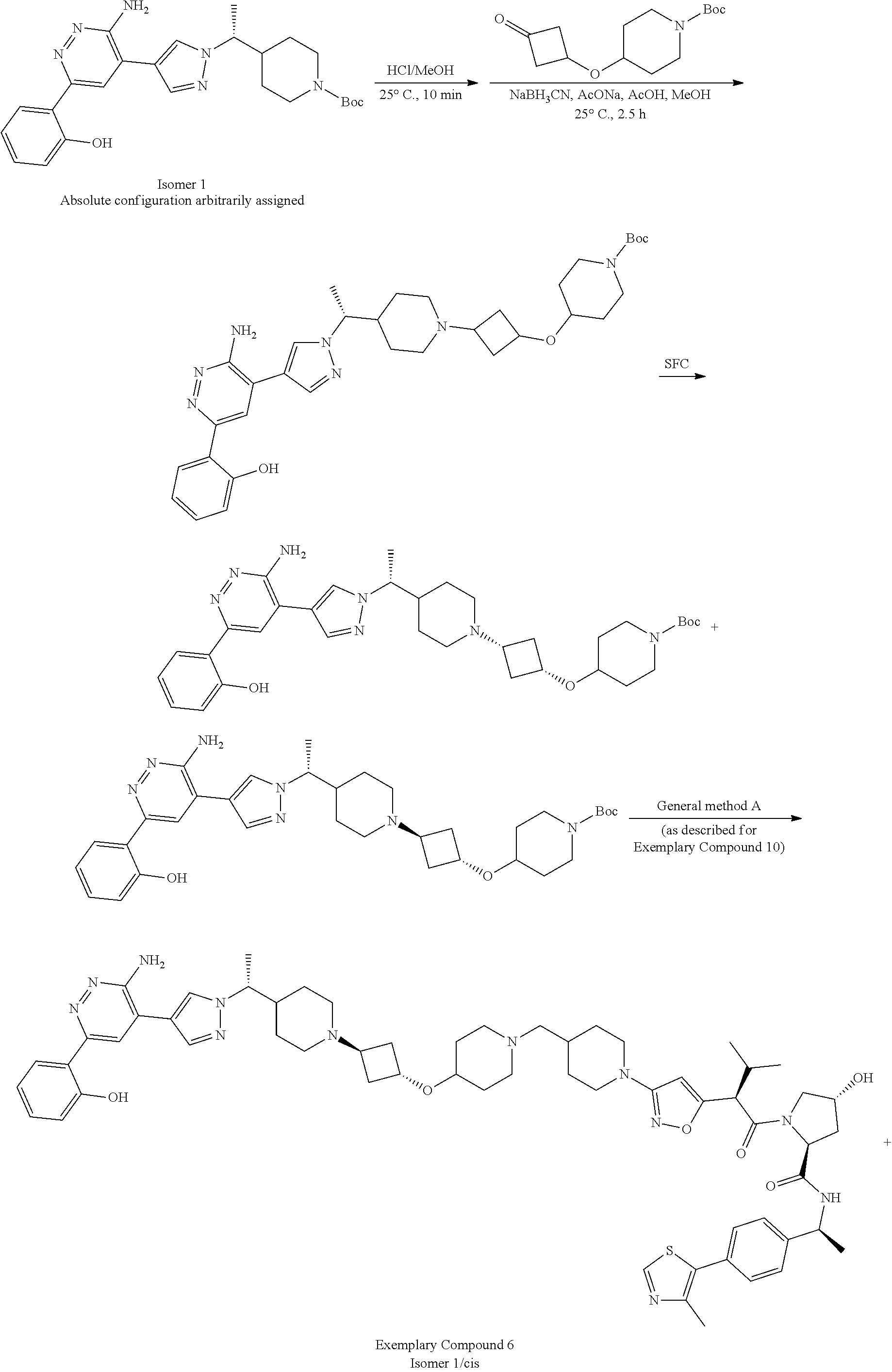

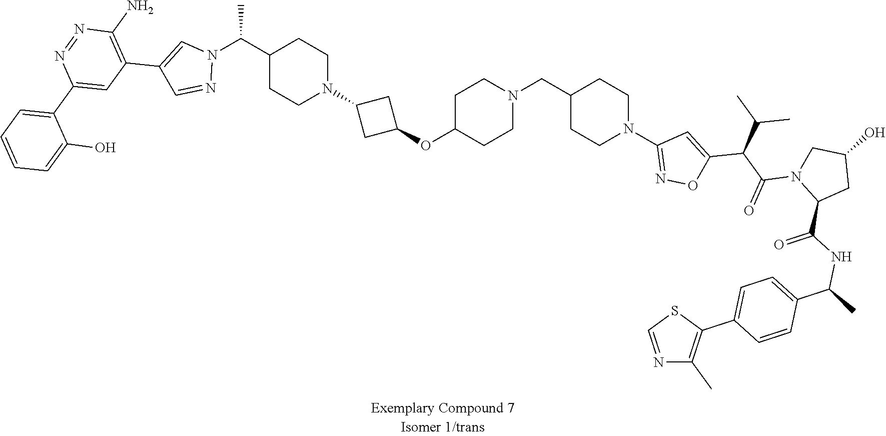 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCC([C@@H](C)n7cc(-c8cc(-c9ccccc9O)nnc8N)cn7)CC6)C5)CC4)CC3)no2)C(C)C)cc1]

Exemplary Compounds 8 and 9 were prepared using analogous procedures.

### Exemplary Synthesis of Compound 10

Compound 10 was prepared according to the scheme below using procedures commonly known to those skilled in the art.

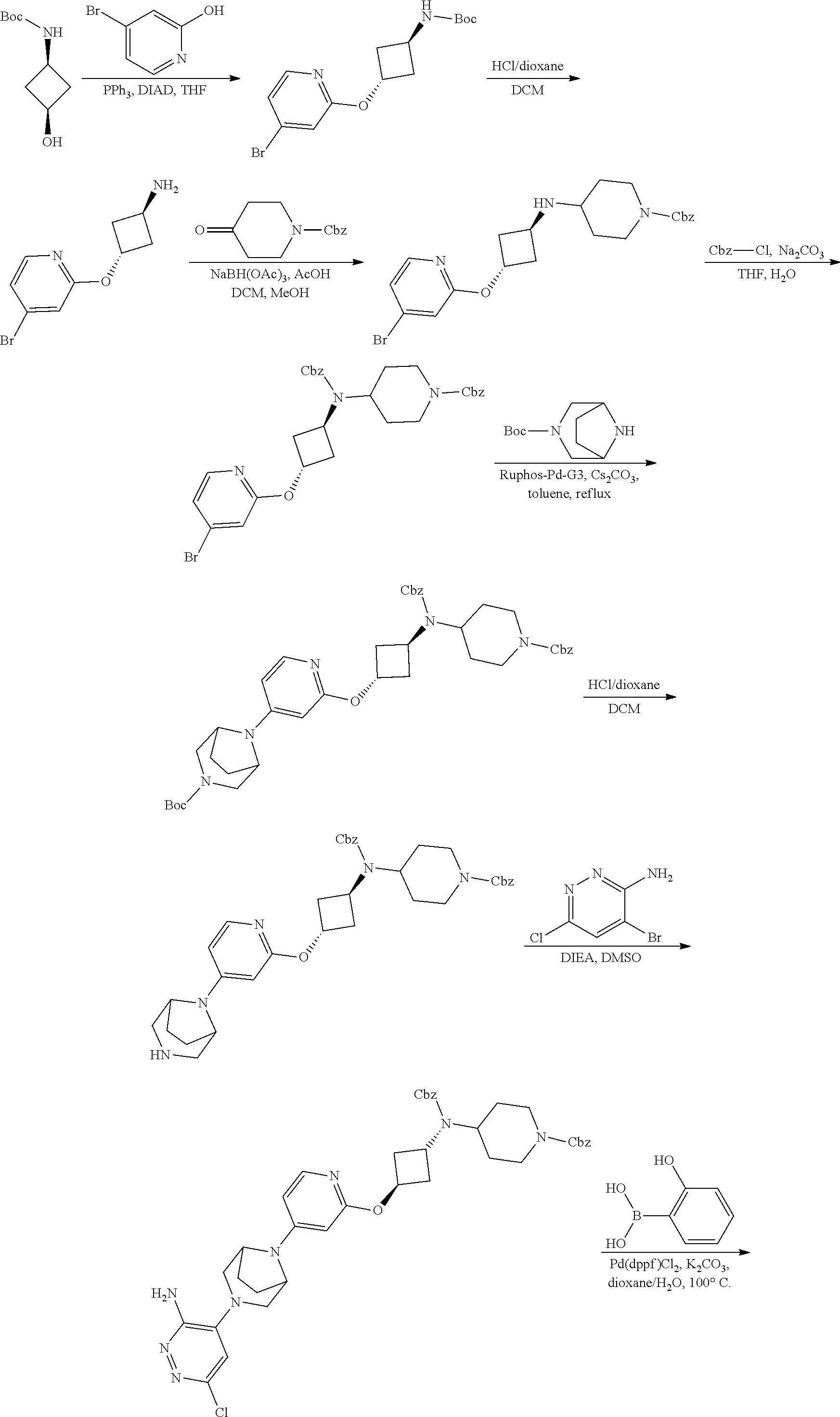

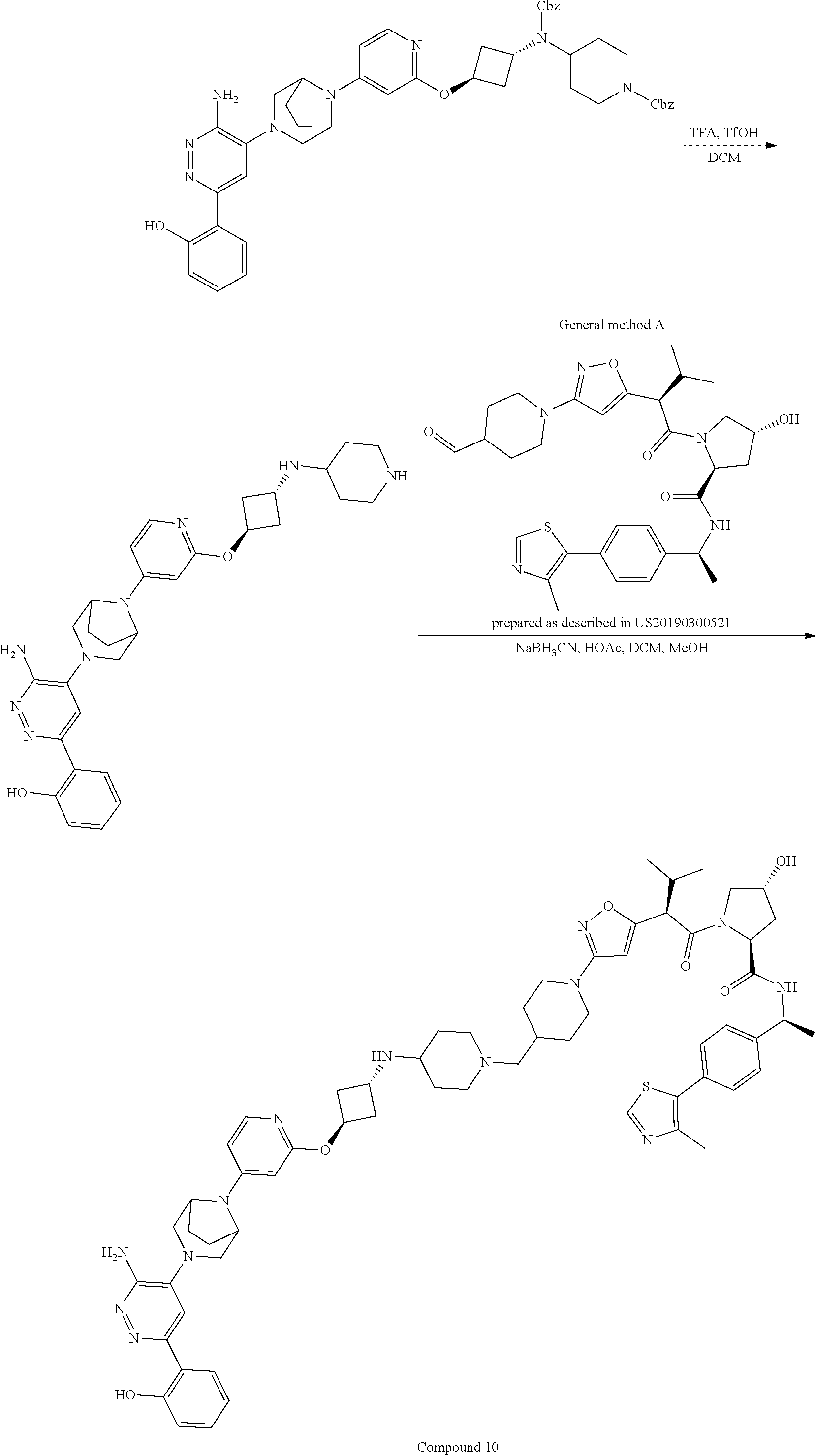 [SMILES: Nc1nnc(-c2ccccc2O)cc1N1CC2CCC(C1)N2c1ccnc(O[C@H]2C[C@H](N(C(=O)OCc3ccccc3)C3CCN(C(=O)OCc4ccccc4)CC3)C2)c1 ; Nc1nnc(-c2ccccc2O)cc1N1CC2CCC(C1)N2c1ccnc(O[C@H]2C[C@H](NC3CCNCC3)C2)c1 ; Cc1ncsc1-c1ccc([C@H](C)NC(=O)[C@@H]2C[C@@H](O)CN2C(=O)[C@@H](c2cc(N3CCC(C=O)CC3)no2)C(C)C)cc1 ; Cc1ncsc1-c1ccc([C@H](C)NC(=O)[C@@H]2C[C@@H](O)CN2C(=O)[C@@H](c2cc(N3CCC(CN4CCC(N[C@H]5C[C@H](Oc6cc(N7C8CCC7CN(c7cc(-c9ccccc9O)nnc7N)C8)ccn6)C5)CC4)CC3)no2)C(C)C)cc1]

### Exemplary Synthesis of Compound 11

Compound 11 was prepared according to the scheme below using procedures commonly known to those skilled in the art.

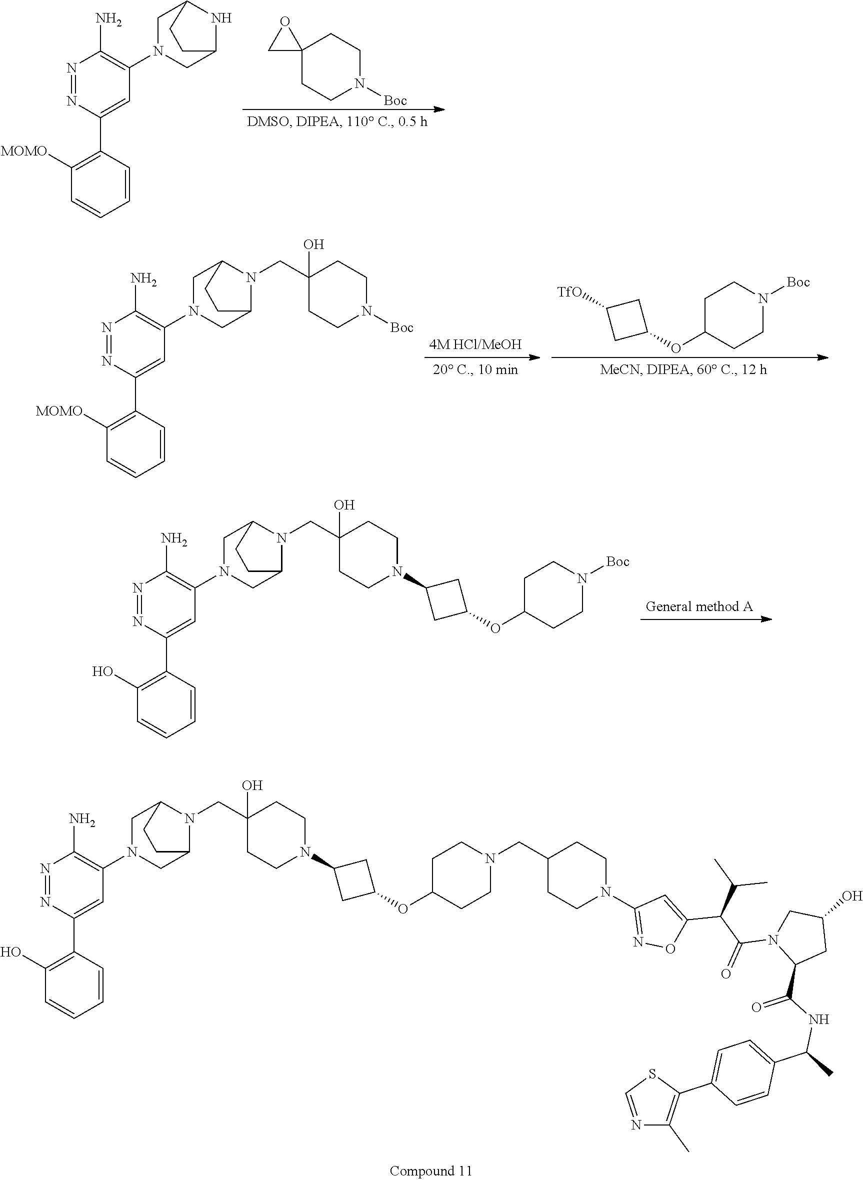

### Exemplary Synthesis of Compound 12

Compound 12 was prepared according to the scheme below using procedures commonly known to those skilled in the art.

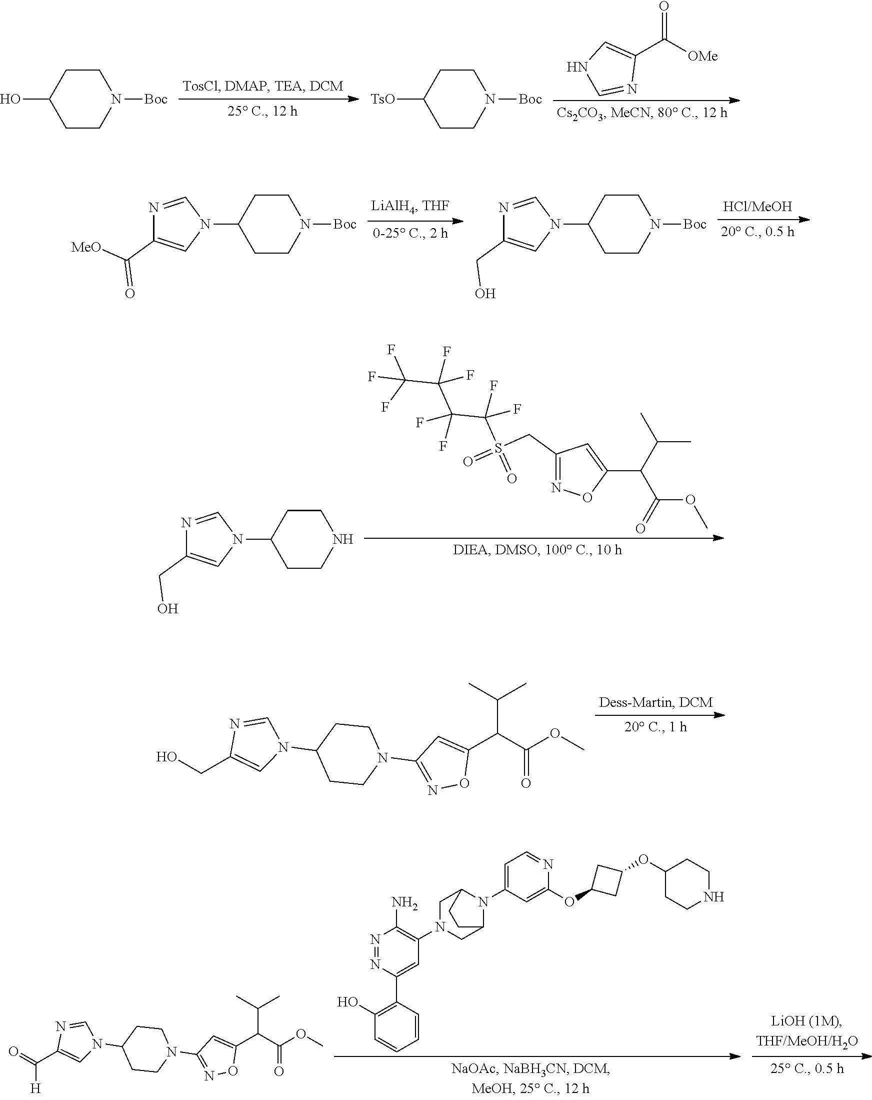

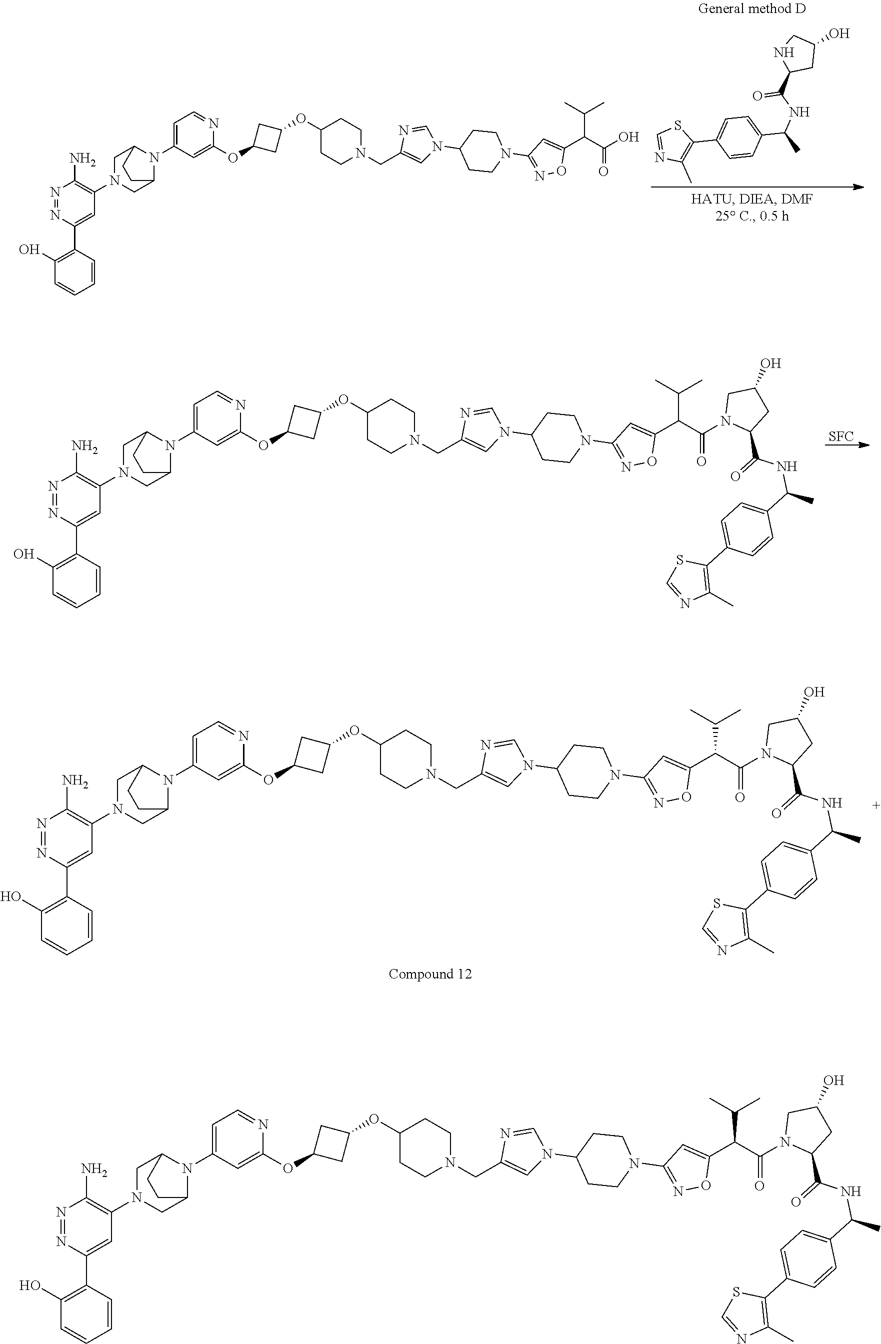

### Exemplary Synthesis of Compound 13

### Step 1

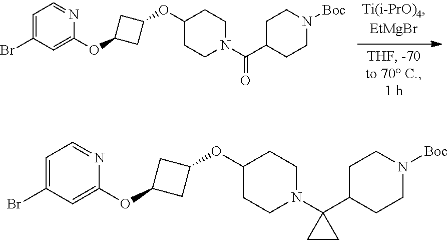 [SMILES: CC(C)(C)OC(=O)N1CCC(C(=O)N2CCC(O[C@H]3C[C@H](Oc4cc(Br)ccn4)C3)CC2)CC1 ; CC(C)(C)OC(=O)N1CCC(C2(N3CCC(O[C@H]4C[C@H](Oc5cc(Br)ccn5)C4)CC3)CC2)CC1]

prepared as described in US 20200038378

To a solution of ethylmagnesium bromide solution (3 M, 55.7 mL, 6 eq) in tetrahydrofuran (120 mL) was added a solution of tert-butyl 4-[4-[3-[(4-bromo-2-pyridyl)oxy]cyclobutoxy] piperidine-1-carbonyl]piperidine-1-carboxylate (15 g, 27.86 mmol, 1 eq) in tetrahydrofuran (60 mL) dropwise at −70° C. under nitrogen. The temperature was maintained below −70° C., and a solution of titanium (IV) isopropoxide (15.83 g, 55.71 mmol, 16.4 mL, 2 eq) in tetrahydrofuran (60 mL) was added. The resulting mixture was heated to 70° C. for 1 hours. The mixture was cooled to 10° C. and quenched with saturated ammonium chloride solution (200 mL). The aqueous phase was extracted with ethyl acetate (100 mL×4). The combined organic phase was washed with brine (50 mL×3), dried with anhydrous sodium sulfate, filtered and concentrated in vacuum. The residue was purified by silica gel chromatography (Petroleum ether/Ethyl acetate=20/1 to 10/1). The resulting material was additionally purified by prep-HPLC (column: Phenomenex luna C18 150*40 mm*15 um; mobile phase: [water (0.1% TFA)-ACN]; B %: 28%-58%, 11 min) to afford tert-butyl 4-[1-[4-[3-[(4-bromo-2-pyridyl)oxy]cyclobutoxy]-1-piperidyl]cyclopropyl] piperidine-1-carboxylate (810 mg, 1.47 mmol, 5% yield) as a yellow solid.

### Step 2

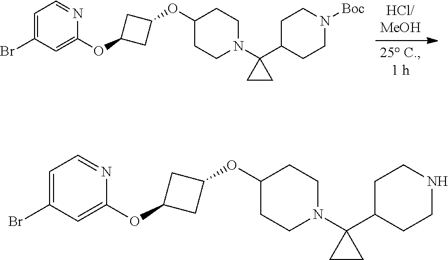 [SMILES: CC(C)(C)OC(=O)N1CCC(C2(N3CCC(O[C@H]4C[C@H](Oc5cc(Br)ccn5)C4)CC3)CC2)CC1 ; Brc1ccnc(O[C@H]2C[C@H](OC3CCN(C4(C5CCNCC5)CC4)CC3)C2)c1]

tert-Butyl 4-[1-[4-[3-[(4-bromo-2-pyridyl)oxy]cyclobutoxy]-1-piperidyl]cyclopropyl]piperidine-1-carboxylate (810 mg, 1.63 mmol, 1 eq) in hydrogen chloride/methanol (4 M, 10 mL, 24.47 eq) was stirred at 25° C. for 1 hours. The mixture was concentrated in reduced pressure at 45° C. The residue was diluted with water (20 mL), pH was adjusted to 8-9 by solid sodium hydrogen carbonate, and the mixture was stirred for 15 min. The aqueous phase was extracted with ethyl acetate (50 mL×4), dried with anhydrous sodium sulfate, filtered and concentrated in vacuum. Crude 4-bromo-2-[3-[[1-[1-(4-piperidyl)cyclopropyl]-4-piperidyl] oxy]cyclobutoxy] pyridine (780 mg) was obtained as a yellow oil and used directly without purification.

### Step 3

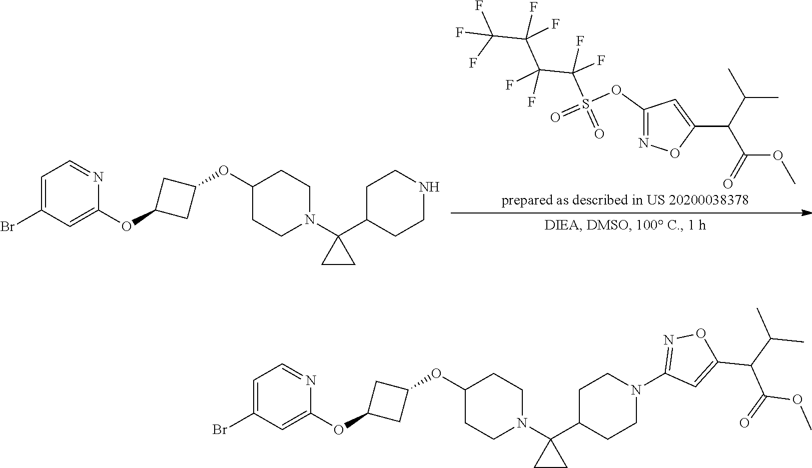 [SMILES: Brc1ccnc(O[C@H]2C[C@H](OC3CCN(C4(C5CCNCC5)CC4)CC3)C2)c1 ; COC(=O)C(c1cc(OS(=O)(=O)C(F)(F)C(F)(F)C(F)(F)C(F)(F)F)no1)C(C)C ; COC(=O)C(c1cc(N2CCC(C3(N4CCC(O[C@H]5C[C@H](Oc6cc(Br)ccn6)C5)CC4)CC3)CC2)no1)C(C)C]

A mixture of 4-bromo-2-[3-[[1-[1-(4-piperidyl)cyclopropyl]-4-piperidyl]oxy]cyclobutoxy] pyridine (780 mg, 1.73 mmol, 1 eq), N,N-diisopropylethylamine (1.12 g, 8.66 mmol, 1.5 mL, 5 eq), and methyl 3-methyl-2-[3-(1,1,2,2,3,3,4,4,4-nonafluorobutylsulfonyloxy)isoxazol-5-yl]butanoate (1.67 g, 3.46 mmol, 2 eq) in dimethyl sulfoxide (5 mL) was stirred at 100° C. for 1 hour. The mixture was cooled to 25° C., diluted with ethyl acetate (100 mL), washed with brine (50 mL×3), dried with anhydrous sodium sulfate, filtered and concentrated in vacuum. The residue was purified by prep-HPLC (column: Phenomenex Synergi Max-RP 250*50 mm*10 um; mobile phase: [water (0.225% FA)-ACN]; B %: 30%-60%, 22 min) to afford methyl 2-[3-[4-[1-[4-[3-[(4-bromo-2-pyridyl)oxy]cyclobutoxy]-1-piperidyl] cyclopropyl]-1-piperidyl]isoxazol-5-yl]-3-methyl-butanoate (510 mg, 0.80 mmol) as a yellow solid.

### Step 4

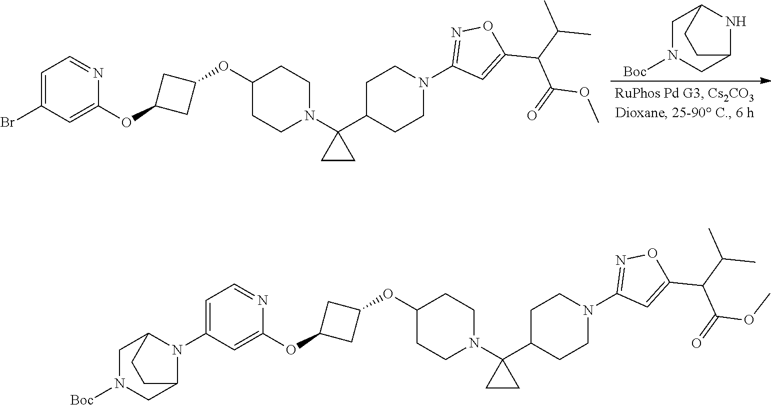 [SMILES: COC(=O)C(c1cc(N2CCC(C3(N4CCC(O[C@H]5C[C@H](Oc6cc(Br)ccn6)C5)CC4)CC3)CC2)no1)C(C)C ; CC(C)(C)OC(=O)N1CC2CCC(C1)N2 ; COC(=O)C(c1cc(N2CCC(C3(N4CCC(O[C@H]5C[C@H](Oc6cc(N7C8CCC7CN(C(=O)OC(C)(C)C)C8)ccn6)C5)CC4)CC3)CC2)no1)C(C)C]

To a mixture of methyl 2-[3-[4-[1-[4-[3-[(4-bromo-2-pyridyl)oxy]cyclobutoxy]-1-piperidyl] cyclopropyl]-1-piperidyl]isoxazol-5-yl]-3-methyl-butanoate (510 mg, 0.80 mmol, 1 eq), tert-butyl (1R,5S)-3,8-diazabicyclo[3.2.1]octane-3-carboxylate (257 mg, 1.21 mmol, 1.5 eq), and cesium carbonate (789 mg, 2.42 mmol, 3.0 eq) in dioxane (10 mL) was added (2-dicyclohexylphosphino-2′,6′-diisopropoxy-1,1′-biphenyl)[2-(2′-amino-1,1′-biphenyl)]palladium(II) methanesulfonate [RuPhos Pd G3] (67 mg, 0.08 mmol, 0.1 eq) in one portion at 25° C. under nitrogen. The mixture was heated to 90° C. and stirred for 6 hours. The mixture was cooled to 25° C. and concentrated under reduced pressure at 45° C. The residue was poured into ice-water (w/w=1/1) (30 mL) and stirred for 15 minutes. The aqueous phase was extracted with ethyl acetate (50 mL×3). The combined organic phase was washed with brine (30 mL×2), dried with anhydrous sodium sulfate, filtered and concentrated in vacuum. The residue was purified by prep-TLC (petroleum ether/ethyl acetate=1/2) to afford tert-butyl 8-[2-[3-[[1-[1-[1-[5-(1-methoxycarbonyl-2-methyl-propyl)isoxazol-3-yl]-4-piperidyl]cyclopropyl]-4-piperidyl]oxy]cyclobutoxy]-4-pyridyl]-3,8-diazabicyclo[3.2.1]octane-3-carboxylate (260 mg, 0.34 mmol) as a yellow oil.

### Step 5

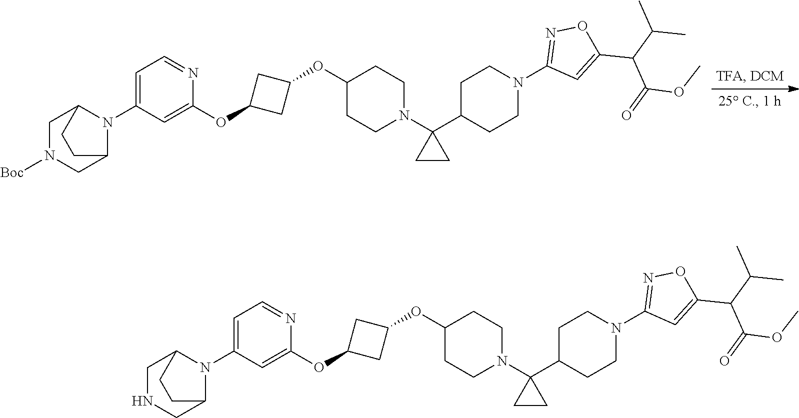 [SMILES: COC(=O)C(c1cc(N2CCC(C3(N4CCC(O[C@H]5C[C@H](Oc6cc(N7C8CCC7CN(C(=O)OC(C)(C)C)C8)ccn6)C5)CC4)CC3)CC2)no1)C(C)C ; COC(=O)C(c1cc(N2CCC(C3(N4CCC(O[C@H]5C[C@H](Oc6cc(N7C8CCC7CNC8)ccn6)C5)CC4)CC3)CC2)no1)C(C)C]

To a mixture of tert-butyl 8-[2-[3-[[1-[1-[1-[5-(1-methoxycarbonyl-2-methyl-propyl)isoxazol-3-yl]-4-piperidyl]cyclopropyl]-4-piperidyl]oxy]cyclobutoxy]-4-pyridyl]-3,8-diazabicyclo[3.2.1]octane-3-carboxylate (260 mg, 0.34 mmol, 1.0 eq) in dichloromethane (10 mL) was added trifluoroacetic acid (4.62 g, 40.52 mmol, 3 mL, 118.90 eq), and the mixture was stirred at 25° C. for 1 hour. The mixture was concentrated under reduced pressure at 45° C. The residue was poured into ice-water (w/w=1/1) (30 mL), and pH was adjusted to 8-9 by solid sodium bicarbonate, and the mixture was stirred for 15 minutes. The aqueous phase was extracted with ethyl acetate (50 mL×3). The combined organic phase was dried with anhydrous sodium sulfate, filtered and concentrated in vacuum. Crude methyl 2-[3-[4-[1-[4-[3-[[4-(3,8-diazabicyclo[3.2.1]octan-8-yl)-2-pyridyl]oxy]cyclobutoxy]-1-piperidyl]cyclopropyl]-1-piperidyl]isoxazol-5-yl]-3-methyl-butanoate (210 mg, 0.31 mmol) was obtained as a yellow oil and used without further purification.

### Step 6

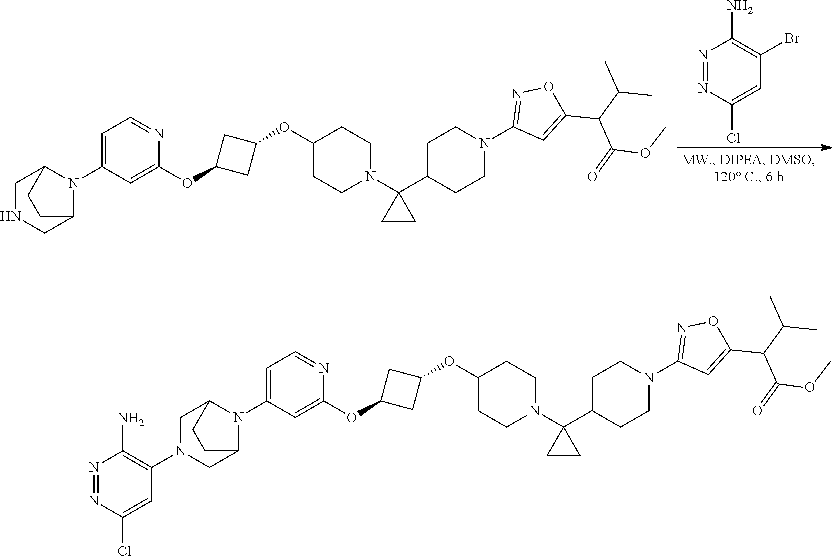 [SMILES: COC(=O)C(c1cc(N2CCC(C3(N4CCC(O[C@H]5C[C@H](Oc6cc(N7C8CCC7CNC8)ccn6)C5)CC4)CC3)CC2)no1)C(C)C ; Nc1nnc(Cl)cc1Br ; COC(=O)C(c1cc(N2CCC(C3(N4CCC(O[C@H]5C[C@H](Oc6cc(N7C8CCC7CN(c7cc(Cl)nnc7N)C8)ccn6)C5)CC4)CC3)CC2)no1)C(C)C]

Methyl 2-[3-[4-[1-[4-[3-[[4-(3,8-diazabicyclo[3.2.1]octan-8-yl)-2-pyridyl]oxy]cyclobutoxy]-1-piperidyl]cyclopropyl]-1-piperidyl]isoxazol-5-yl]-3-methyl-butanoate (150 mg, 0.22 mmol, 1.0 eq), 4-bromo-6-chloro-pyridazin-3-amine (235 mg, 1.13 mmol, 5.0 eq) and N,N-diisopropylethylamine (146 mg, 1.13 mmol, 5.0 eq) were taken up in a microwave tube in dimethylsulfoxide (6 mL). The sealed tube was heated at 120° C. for 6 hours under microwave irradiation. The mixture was cooled to 25° C., diluted with ethyl acetate (60 mL), washed with brine (30 mL×3), dried with anhydrous sodium sulfate, filtered and concentrated in vacuum. The residue was purified by prep-TLC (dichloromethane:methanol=10:1) to afford methyl 2-[3-[4-[1-[4-[3-[[4-[3-(3-amino-6-chloro-pyridazin-4-yl)-3,8-diazabicyclo[3.2.1]octan-8-yl]-2-pyridyl]oxy]cyclobutoxy]-1-piperidyl]cyclopropyl]-1-piperidyl]isoxazol-5-yl]-3-methyl-butanoate (120 mg, 0.15 mmol) as a yellow oil.

### Step 7

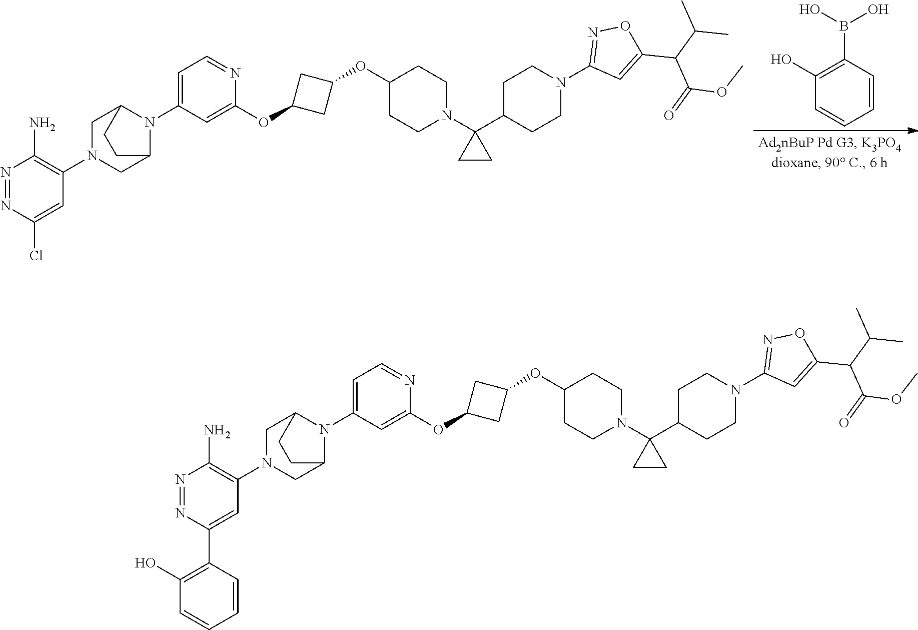

A mixture of methyl 2-[3-[4-[1-[4-[3-[[4-[3-(3-amino-6-chloro-pyridazin-4-yl)-3,8-diazabicyclo[3.2.1] octan-8-yl]-2-pyridyl]oxy]cyclobutoxy]-1-piperidyl]cyclopropyl]-1-piperidyl]isoxazol-5-yl]-3-methyl-butanoate (150 mg, 0.18 mmol, 1.0 eq), (2-hydroxyphenyl)boronic acid (52 mg, 0.37 mmol, 2.0 eq), methanesulfonato(diadamantyl-n-butylphosphino)-2′-amino-1,1′-biphenyl-2-yl) palladium(II) dichloromethane (13 mg, 0.02 mmol, 0.1 eq) and aqueous potassium phosphate (1.5 M, 0.4 mL, 3 eq) in dioxane (6 mL) was degassed and then heated to 90° C. for 6 hours under nitrogen. The mixture was cooled to 25° C., diluted with ethyl acetate (60 mL), washed with brine (30 mL×3), dried with anhydrous sodium sulfate, filtered and concentrated in vacuum. The residue was purified by prep-TLC (dichloromethane:methanol=10:1) to afford methyl 2-[3-[4-[1-[4-[3-[[4-[3-[3-amino-6-(2-hydroxyphenyl)pyridazin-4-yl]-3,8-diazabicyclo[3.2.1]octan-8-yl]-2-pyridyl]oxy]cyclobutoxy]-1-piperidyl]cyclopropyl]-1-piperidyl]isoxazol-5-yl]-3-methyl-butanoate (110 mg, 0.12 mmol) as a yellow oil.

Methyl 2-[3-[4-[1-[4-[3-[[4-[3-[3-amino-6-(2-hydroxyphenyl)pyridazin-4-yl]-3,8-diazabicyclo[3.2.1]octan-8-yl]-2-pyridyl]oxy]cyclobutoxy]-1-piperidyl]cyclopropyl]-1-piperidyl]isoxazol-5-yl]-3-methyl-butanoate was converted to the title compound according to the scheme below using procedures described above as well as those commonly known to those skilled in the art.

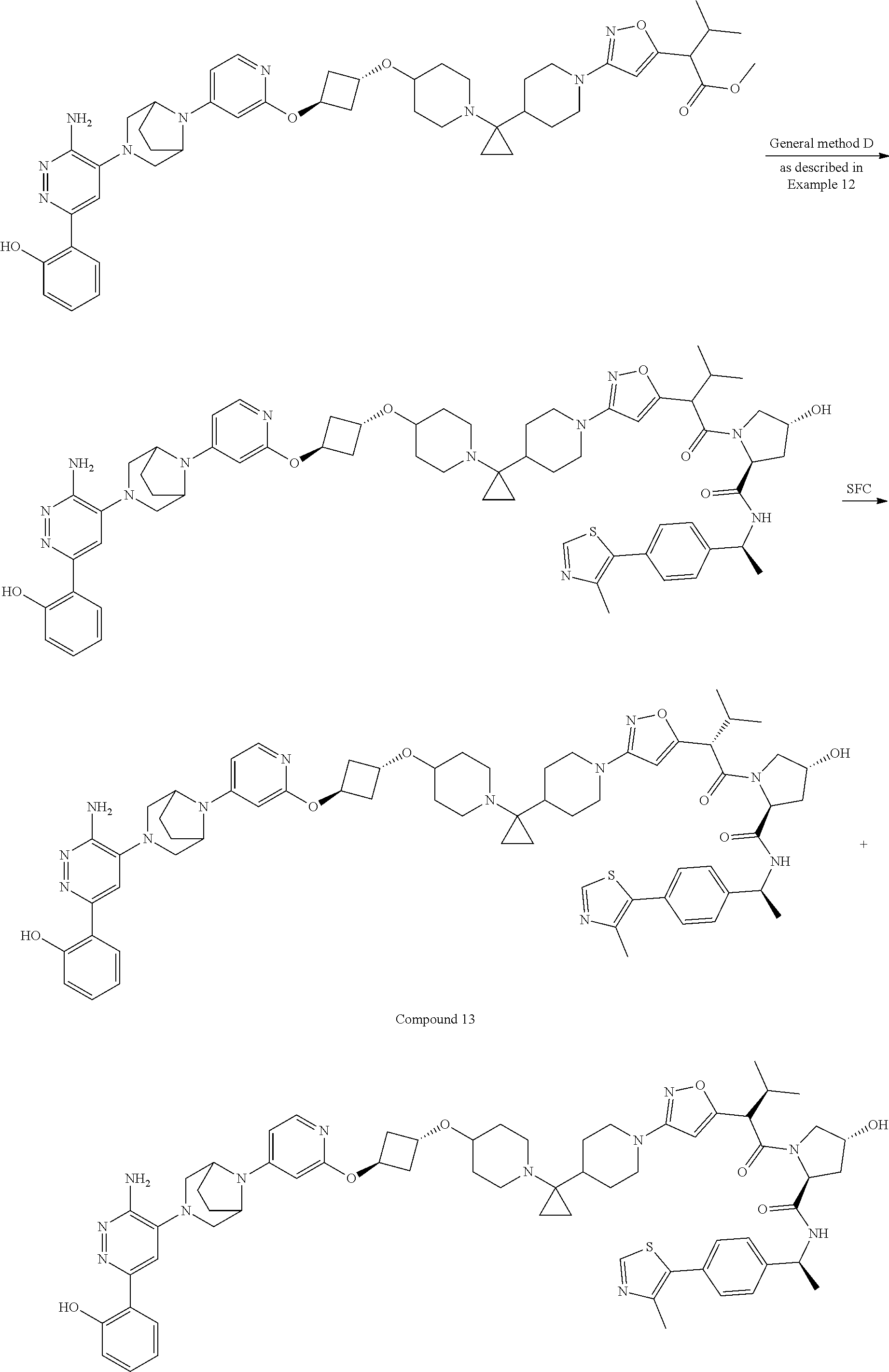

### Exemplary Synthesis of Compound 14

Compound 14 was prepared according to the scheme below using procedures analogous to those described in Compound 13, as well as those commonly known to skilled in the art.

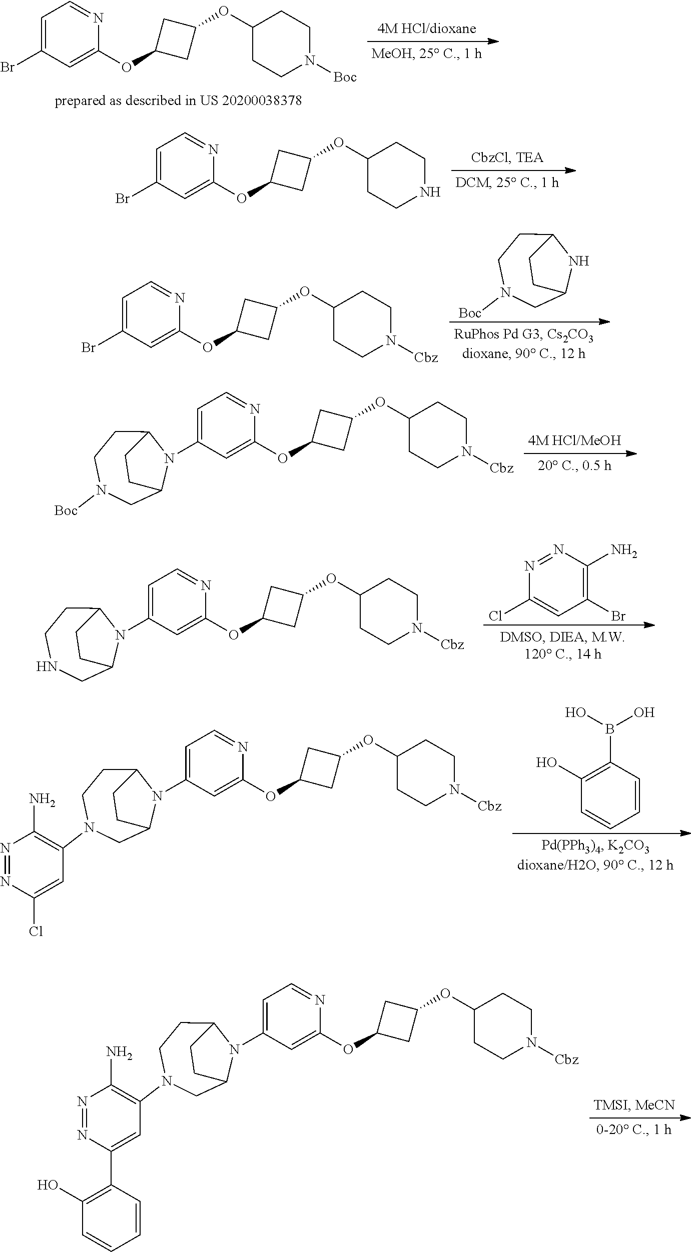

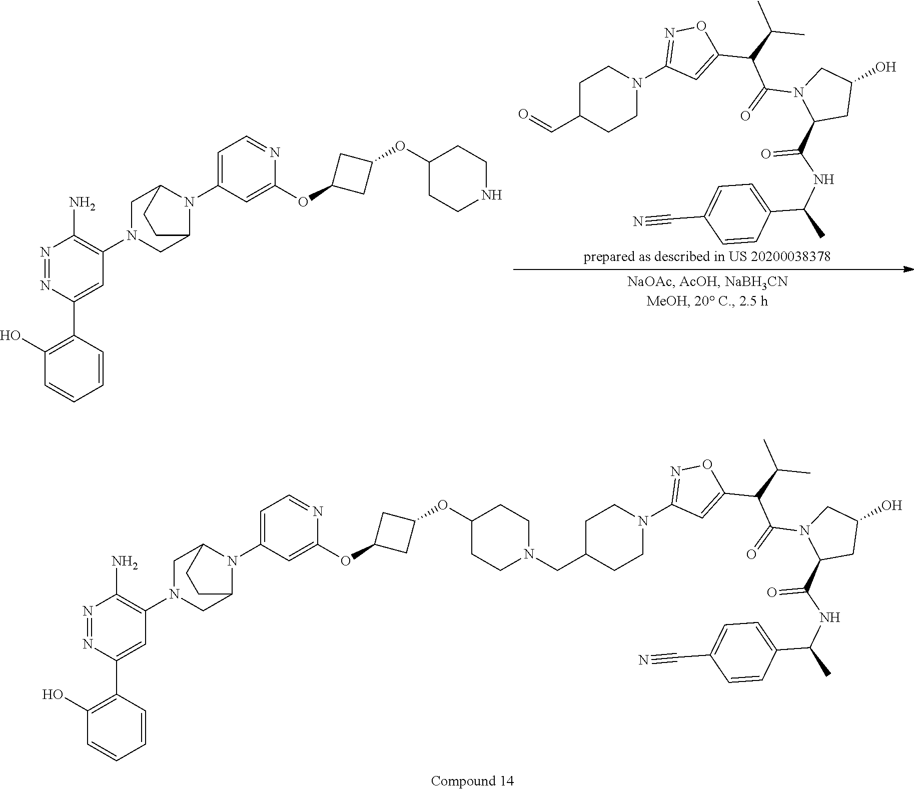

Compounds 15, 16, 17, and 26 were prepared using analogous procedures.

### Exemplary Synthesis of Compound 19

Compound 19 was prepared according to the scheme below using procedures commonly known to those skilled in the art.

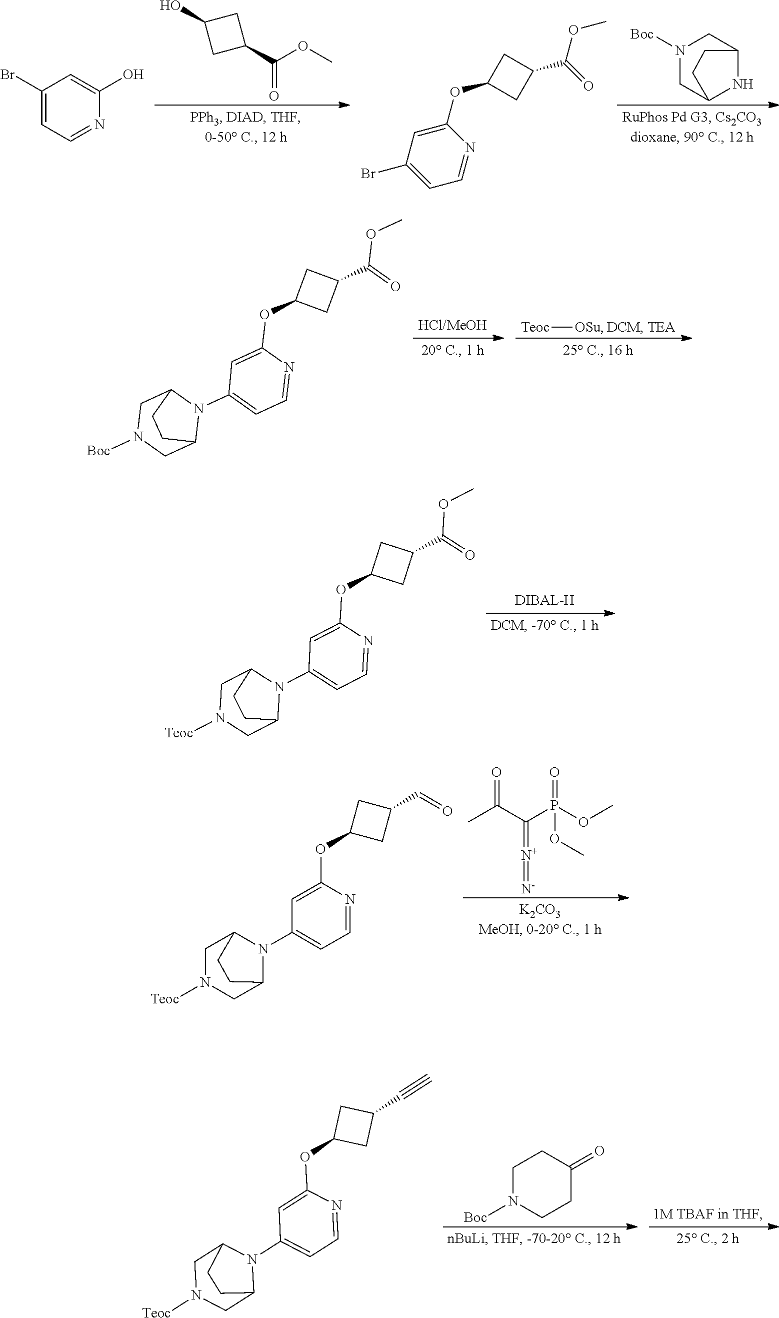

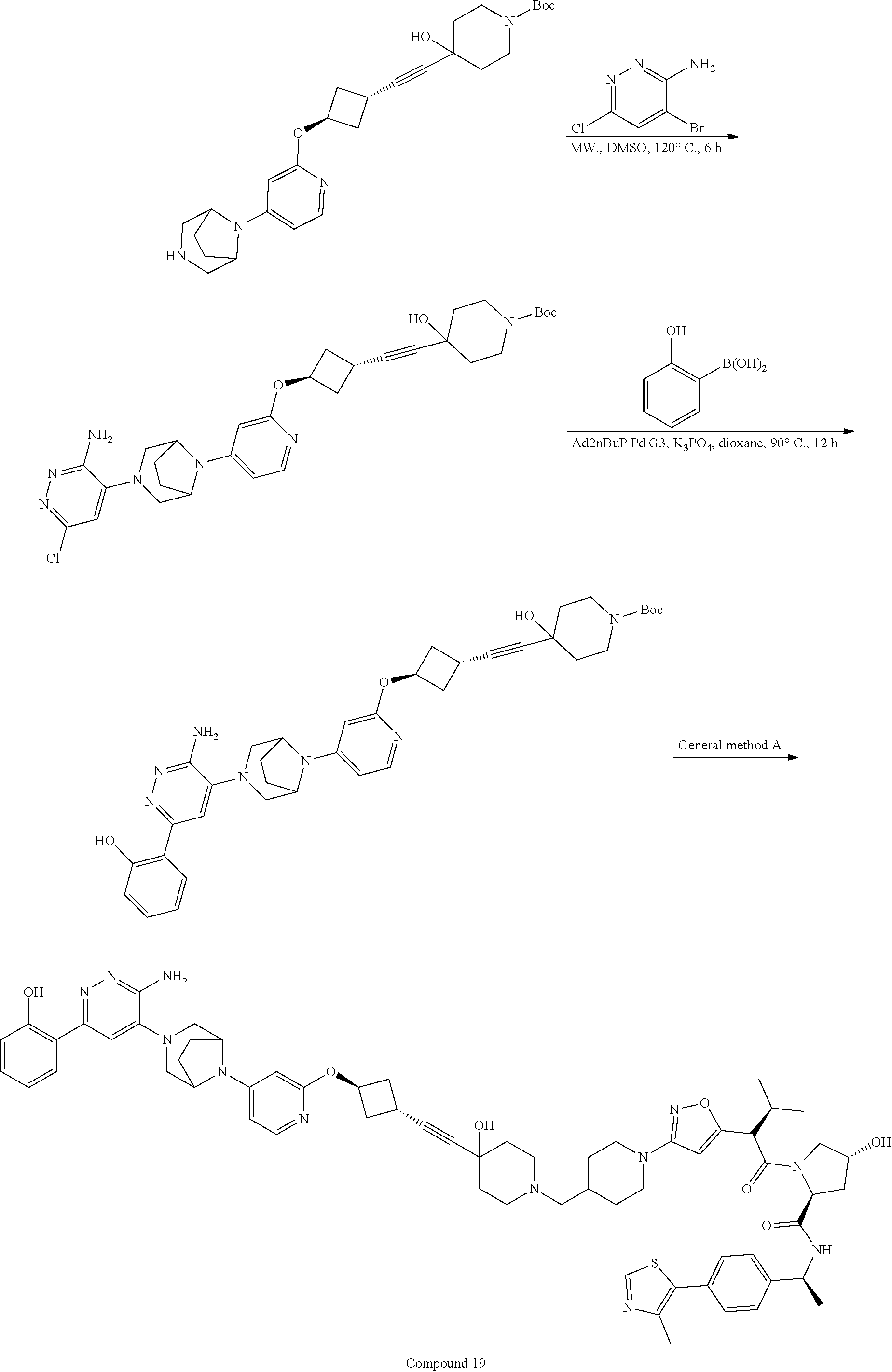

### Exemplary Synthesis of Compound 20

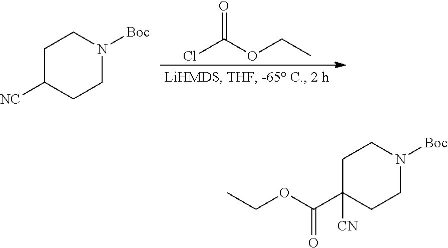

To a solution of tert-butyl 4-cyanopiperidine-1-carboxylate (2.1 g, 9.99 mmol, 1 eq) in tetrahydrofuran (25 mL) was added lithium bis(trimethylsilyl)amide (1 M in tetrahydrofuran, 20 mL, 2 eq) dropwise at −65° C. and then stirred at this temperature for 1 hour. Then a solution of ethyl chloroformate (2.17 g, 19.97 mmol, 1.9 mL, 2 eq) in tetrahydrofuran (5 mL) was added at −65° C., and the mixture was stirred at this temperature for 1 hour. The reaction mixture was quenched with saturated sodium bicarbonate aqueous solution (50 mL) at 0° C. and extracted with ethyl acetate (30 mL×3), the combined organic layers were washed with brine (20 mL), dried over anhydrous sodium sulfate, filtered and concentrated. The residue was purified by silica gel column chromatography (petroleum ether/ethyl acetate=50/1 to 3/1). 1-(tert-butyl) 4-ethyl 4-cyanopiperidine-1,4-dicarboxylate (2.48 g, 8.78 mmol, 88% yield) was obtained as a colorless oil.

The title compound was prepared according to the scheme below using procedures generally known to those skilled in the art.

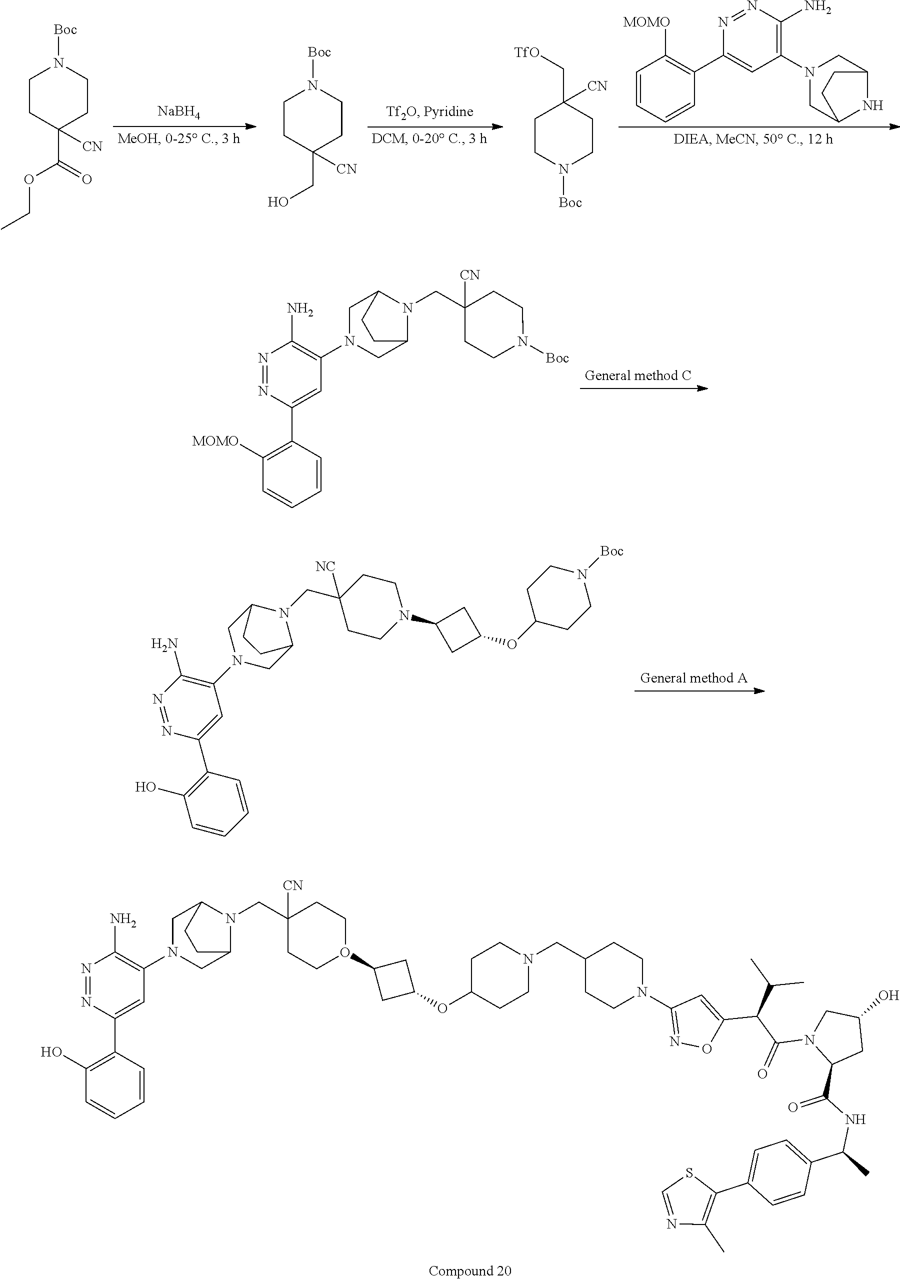

### Exemplary Synthesis of Compound 22

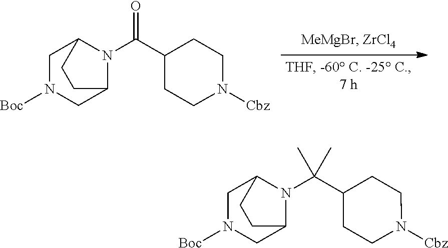 [SMILES: CC(C)(C)OC(=O)N1CC2CCC(C1)N2C(=O)C1CCN(C(=O)OCc2ccccc2)CC1 ; CC(C)(C)OC(=O)N1CC2CCC(C1)N2C(C)(C)C1CCN(C(=O)OCc2ccccc2)CC1]

To a mixture of zirconium(iv) chloride (3.61 g, 15.47 mmol, 1.2 eq) in tetrahydrofuran (120 mL) was added a solution of tert-butyl 8-(1-benzyloxycarbonylpiperidine-4-carbonyl)-3,8-diazabicyclo[3.2.1]octane-3-carboxylate (5.9 g, 12.89 mmol, 1 eq) in tetrahydrofuran (60 mL) dropwise at −60° C. over a period of 0.5 hours under nitrogen. Then to the mixture was added methylmagnesium bromide solution (3 M, 25.8 mL, 6 eq) at −60° C., and the mixture was stirred for 0.5 hours. The resulting mixture was then warmed up to 25° C. and stirred for 6 hours. The reaction mixture was diluted with water (300 mL) and extracted with ethyl acetate (500 mL×2). The combined organic phase was washed with brine (200 mL), dried with anhydrous sodium sulfate, filtered and concentrated in vacuum. The residue was purified by prep-HPLC (column: Phenomenex luna C18 (250*70 mm, 15 um); mobile phase: [water (0.225% FA)-ACN]; B %: 30ACN %-60ACN %, 30 min). Compound tert-butyl 8-[1-(1-benzyloxycarbonyl-4-piperidyl)-1-methyl-ethyl]-3,8-diazabicyclo[3.2.1] octane-3-carboxylate (2.5 g, 5.30 mmol, 41% yield) was obtained as a colorless oil.

Compound 22 was prepared according to the scheme below using procedures described above, as well as procedures generally known to those skilled in the art.

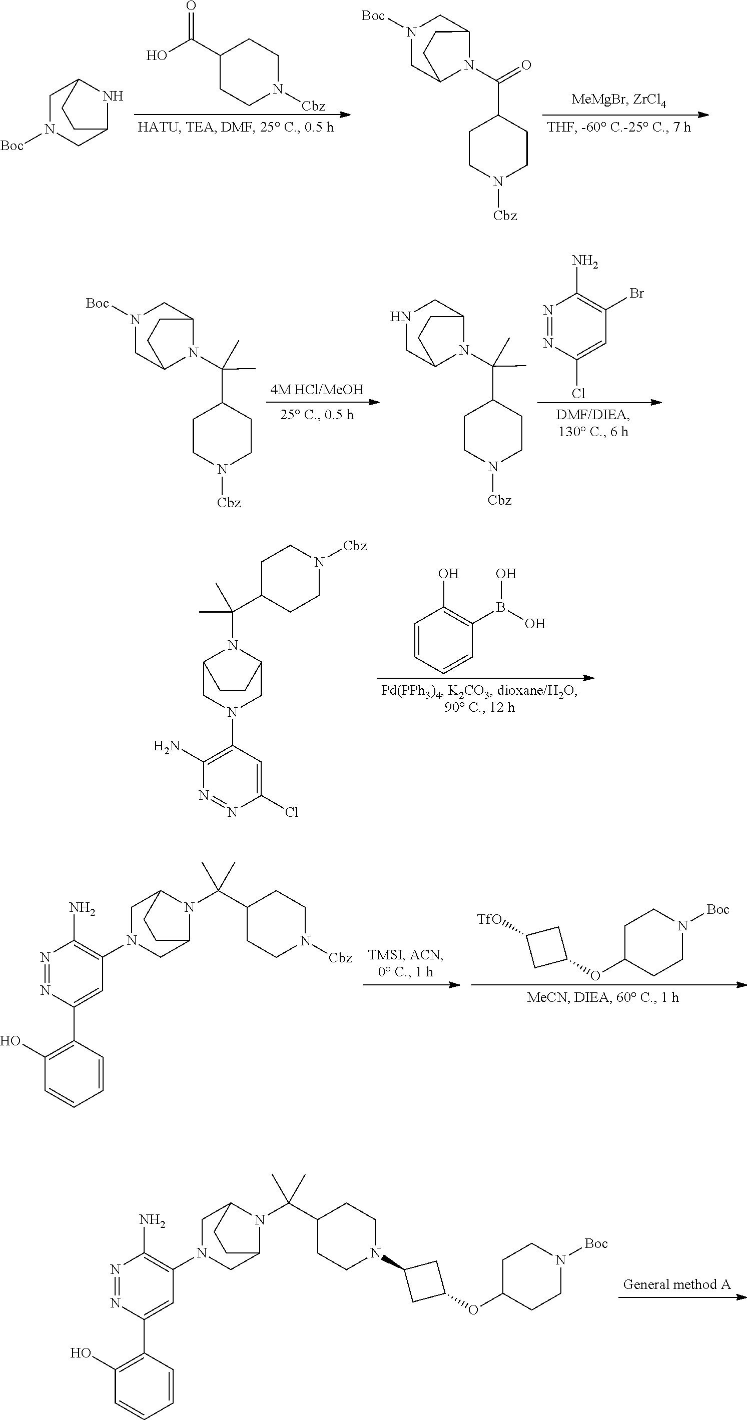

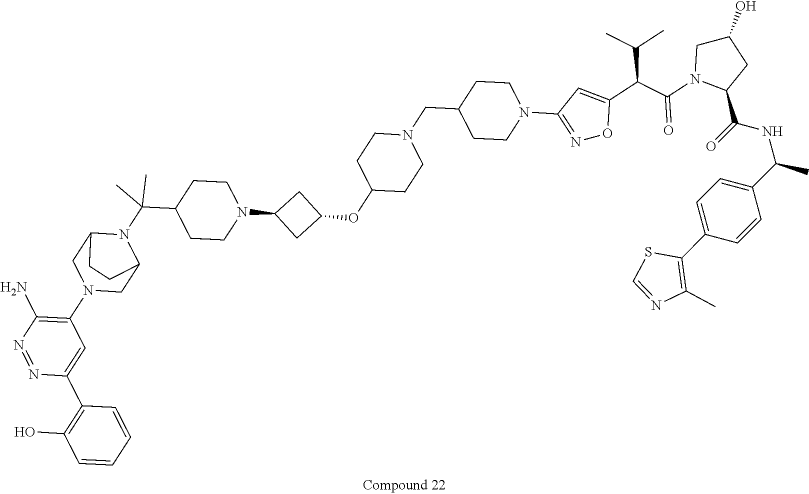 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCC(C(C)(C)N7C8CCC7CN(c7cc(-c9ccccc9O)nnc7N)C8)CC6)C5)CC4)CC3)no2)C(C)C)cc1]

### Exemplary Synthesis of Compound 23

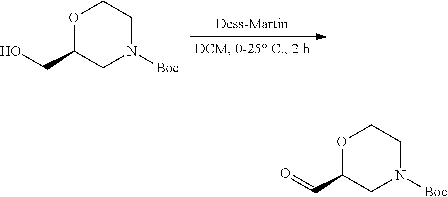 [SMILES: CC(C)(C)OC(=O)N1CCO[C@H](CO)C1 ; CC(C)(C)OC(=O)N1CCO[C@H](C=O)C1]

To a solution of tert-butyl (2S)-2-(hydroxymethyl)morpholine-4-carboxylate (500 mg, 2.30 mmol, 1 eq) in dichloromethane (10 mL) was added Dess-Martin periodinane (1.17 g, 2.76 mmol, 1.2 eq) at 0° C. The mixture was stirred at 25° C. for 2 hours. The mixture was cooled down to 0° C., quenched with saturated sodium thiosulfate solution (50 mL) and saturated sodium bicarbonate solution. The mixture was stirred for 15 minutes and extracted with dichloromethane (2×50 mL). The organic layer was washed with brine (50 mL), dried with sodium sulfate, and concentrated in vacuo. The residue was purified by silica gel chromatography (petroleum ether/ethyl acetate=20/1 to 1/1). Compound tert-butyl (2S)-2-formylmorpholine-4-carboxylate (400 mg, 1.86 mmol) was obtained as a colorless oil.

tert-Butyl (2S)-2-formylmorpholine-4-carboxylate was converted to the title compound according to the scheme below using procedures described above.

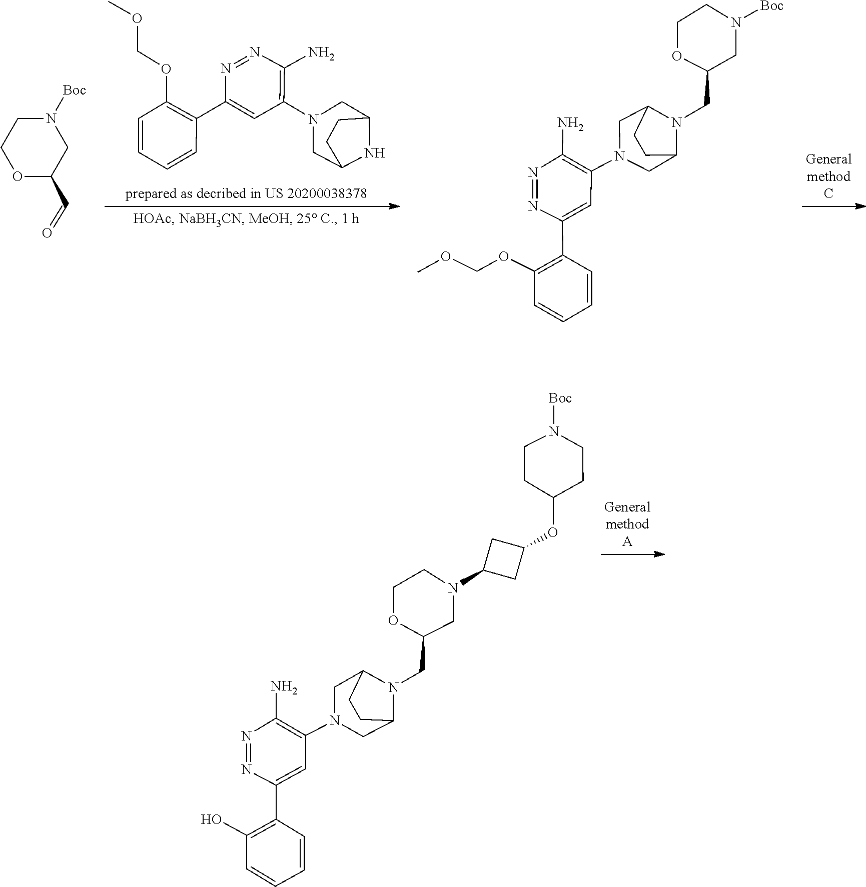 [SMILES: CC(C)(C)OC(=O)N1CCO[C@H](C=O)C1 ; COCOc1ccccc1-c1cc(N2CC3CCC(C2)N3)c(N)nn1 ; COCOc1ccccc1-c1cc(N2CC3CCC(C2)N3C[C@@H]2CN(C(=O)OC(C)(C)C)CCO2)c(N)nn1 ; CC(C)(C)OC(=O)N1CCC(O[C@H]2C[C@H](N3CCO[C@H](CN4C5CCC4CN(c4cc(-c6ccccc6O)nnc4N)C5)C3)C2)CC1]

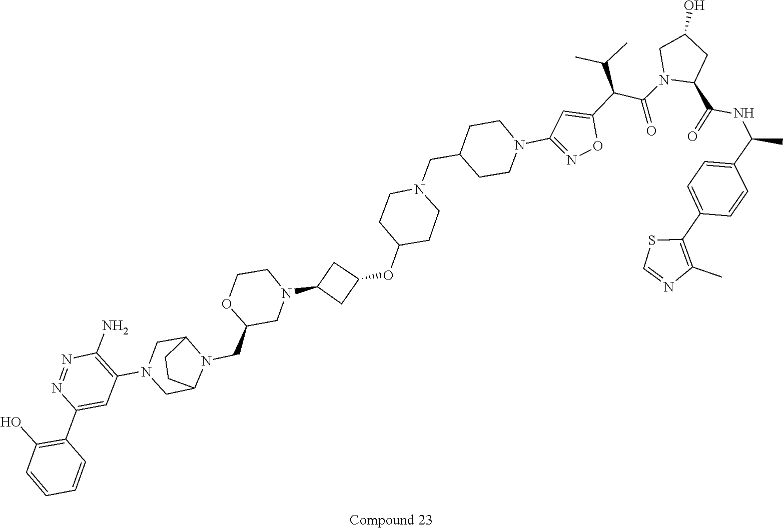 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCO[C@H](CN7C8CCC7CN(c7cc(-c9ccccc9O)nnc7N)C8)C6)C5)CC4)CC3)no2)C(C)C)cc1]

### Exemplary Synthesis of Compound 24

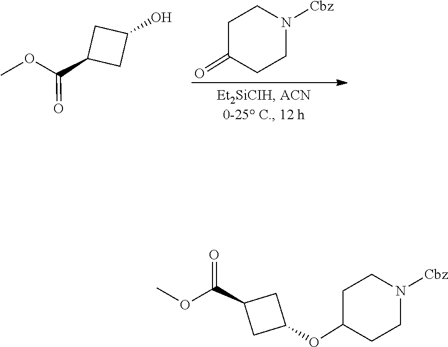

A mixture of methyl 3-hydroxycyclobutanecarboxylate (1 g, 7.68 mmol, 1 eq) and benzyl 4-oxopiperidine-1-carboxylate (1.97 g, 8.45 mmol, 1.6 mL, 1.1 eq) in acetonitrile (10 mL) was degassed and purged with nitrogen for 3 times. To it was then added chloro(dimethyl)silane (727 mg, 7.68 mmol, 1 eq) at 0° C. The mixture was stirred at 25° C. for 12 hours under nitrogen atmosphere. The reaction mixture was diluted with water 100 mL and extracted with ethyl acetate (100 mL×2). The combined organic phase was washed with saturated brine (100 mL×2), dried with anhydrous sodium sulfate, filtered and concentrated in vacuum. The residue was purified by prep-HPLC (column: Phenomenex luna C18 150*25 mm*10 um; mobile phase: [water (0.1% TFA)-ACN]; B %: 40%-70%, 10 min). Compound benzyl 4-(3-methoxycarbonylcyclobutoxy)piperidine-1-carboxylate (500 mg, 1.44 mmol, 18% yield) was obtained as a colorless oil.

Benzyl 4-(3-methoxycarbonylcyclobutoxy)piperidine-1-carboxylate was converted to tert-butyl 4-((1r, 3r)-3-formylcyclobutoxy)piperidine-1-carboxylate as shown in the scheme below.

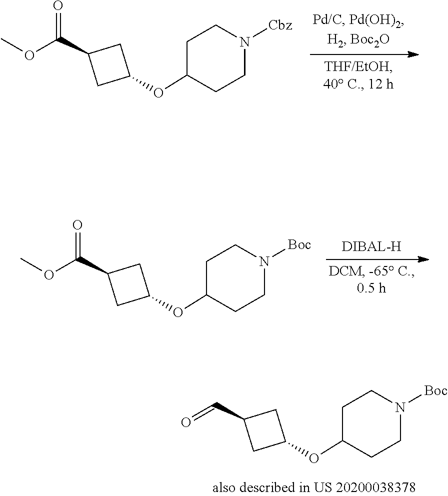 [SMILES: COC(=O)[C@H]1C[C@H](OC2CCN(C(=O)OCc3ccccc3)CC2)C1 ; COC(=O)[C@H]1C[C@H](OC2CCN(C(=O)OC(C)(C)C)CC2)C1 ; CC(C)(C)OC(=O)N1CCC(O[C@H]2C[C@H](C=O)C2)CC1]

Compound 24 was prepared according to the scheme below using procedures described or referenced above, as well as general procedures known to those skilled in the art.

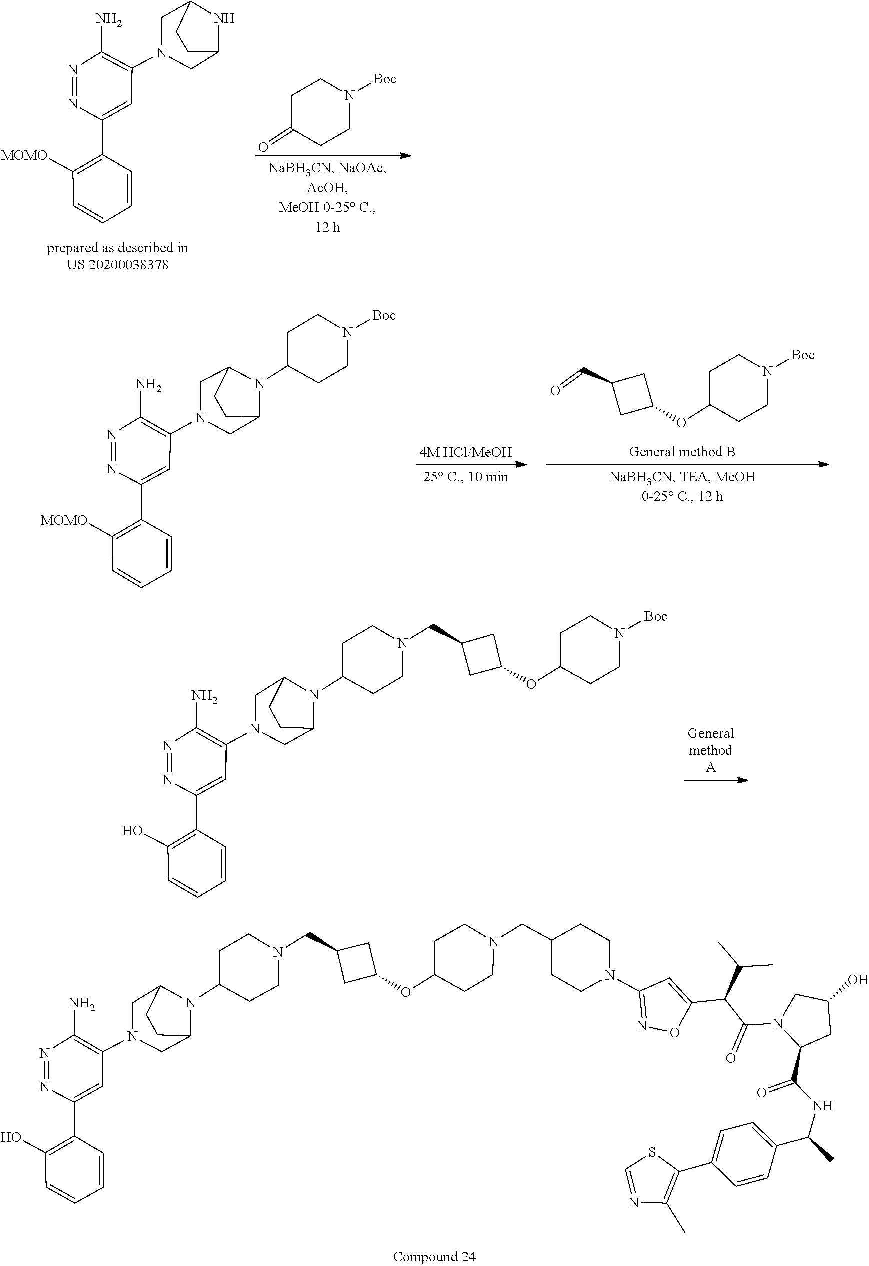

### Exemplary Synthesis of Compound 25

Compound 25 was prepared according to the scheme below using procedures described above.

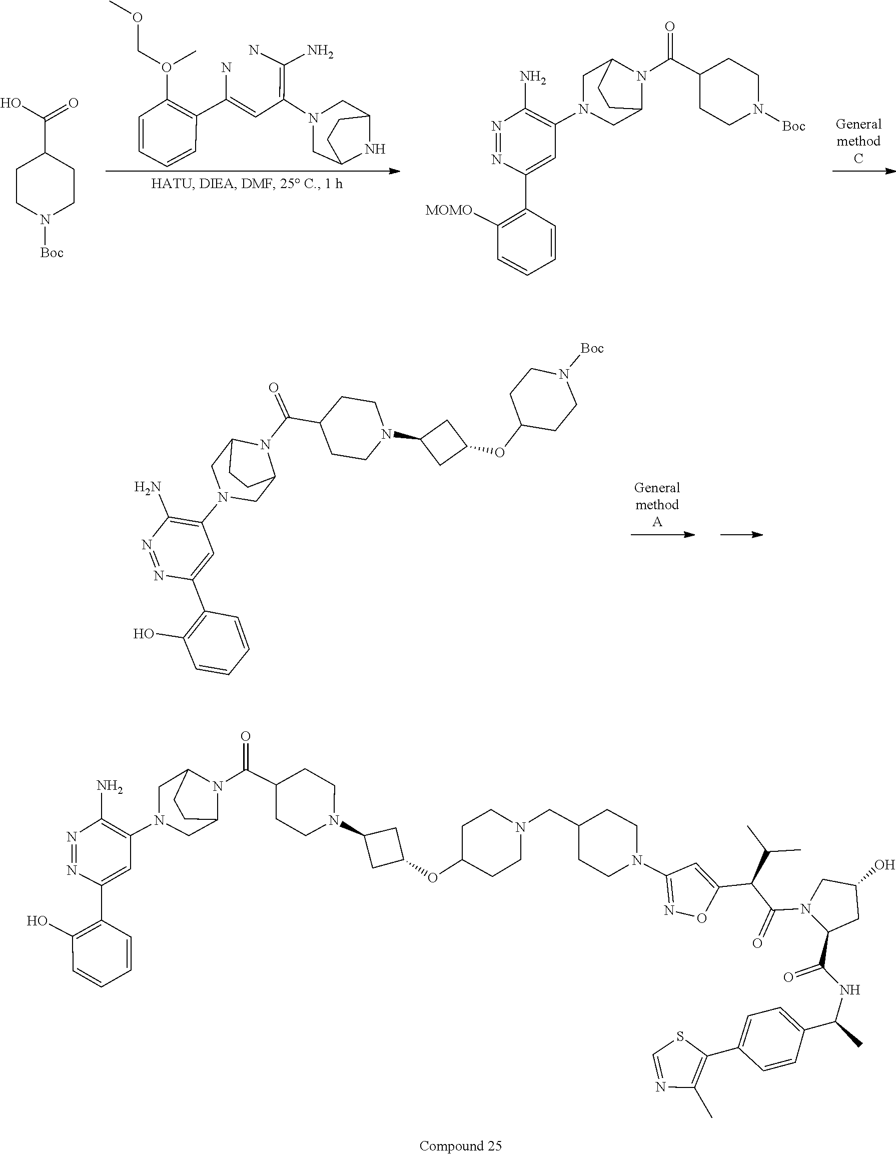

### Exemplary Synthesis of Compound 27

Compound 27 was prepared according to the scheme below using procedures described above as well as those generally known to skilled in the art.

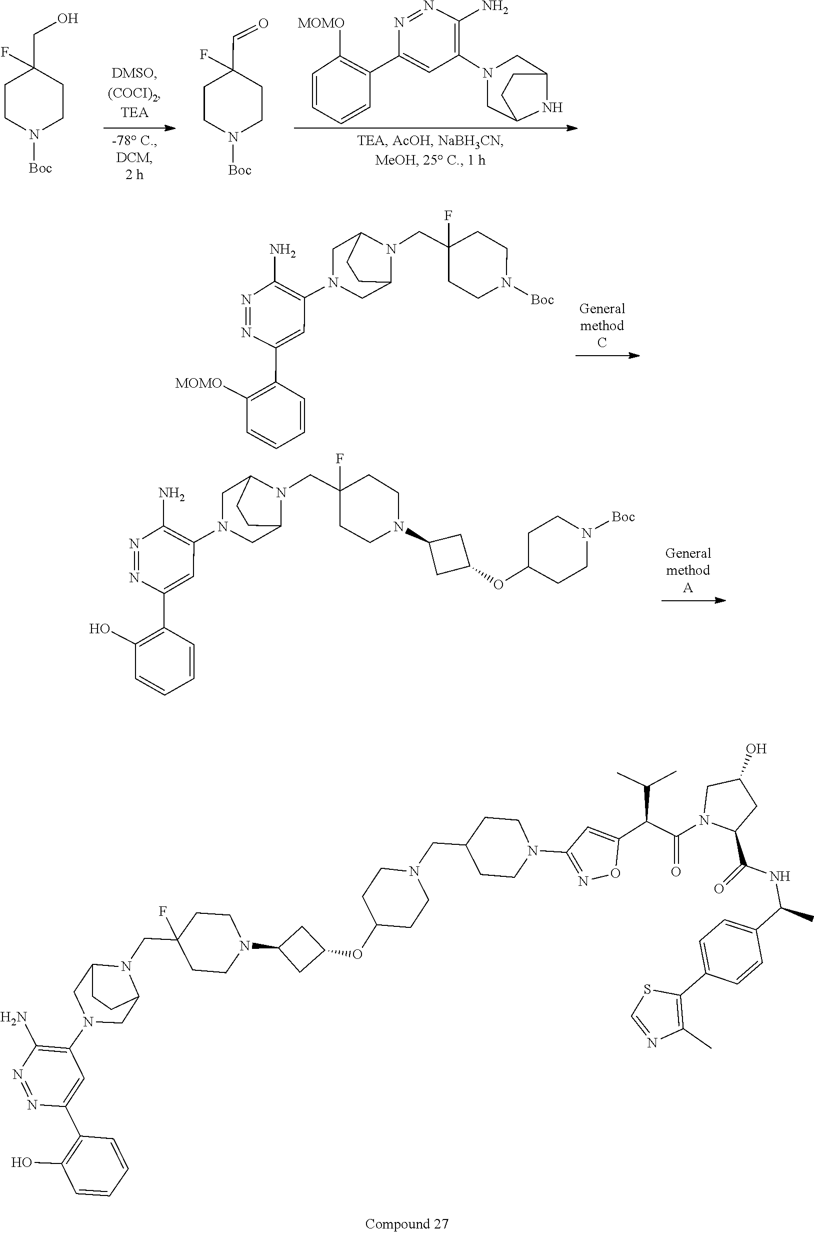

### Exemplary Synthesis of Compound 28

Compound 28 was prepared according to the scheme below using procedures described above.

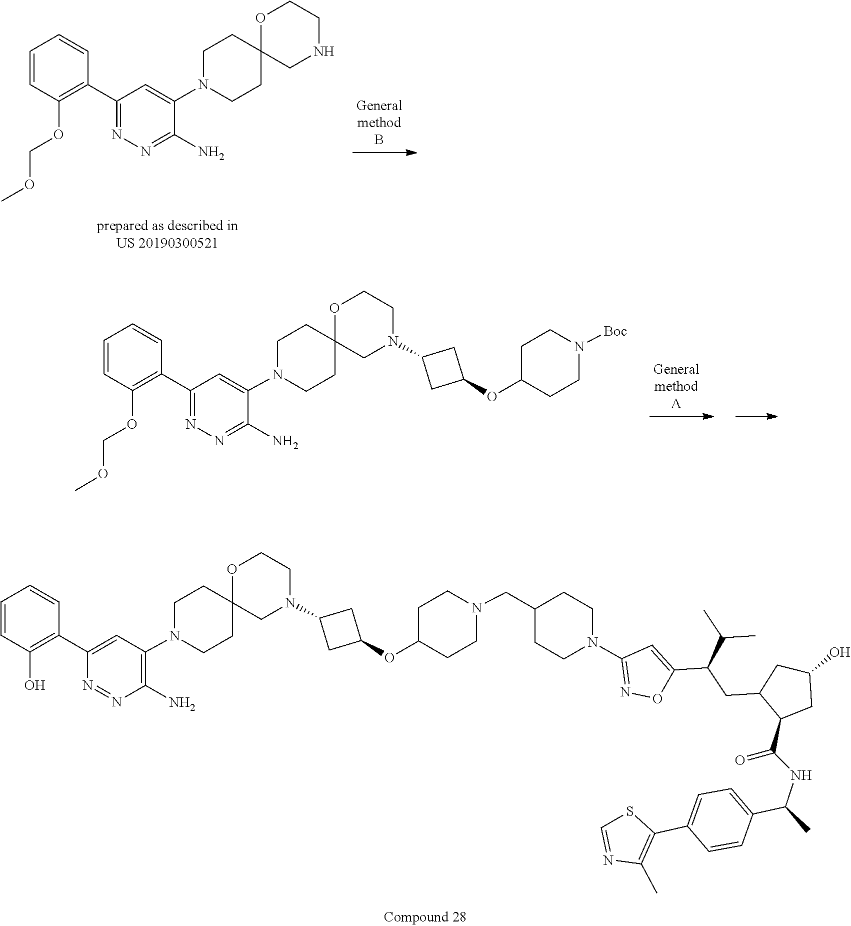 [SMILES: COCOc1ccccc1-c1cc(N2CCC3(CC2)CNCCO3)c(N)nn1 ; COCOc1ccccc1-c1cc(N2CCC3(CC2)CN([C@H]2C[C@H](OC4CCN(C(=O)OC(C)(C)C)CC4)C2)CCO3)c(N)nn1 ; Cc1ncsc1-c1ccc([C@H](C)NC(=O)[C@@H]2C[C@@H](O)CC2C[C@@H](c2cc(N3CCC(CN4CCC(O[C@H]5C[C@H](N6CCOC7(CCN(c8cc(-c9ccccc9O)nnc8N)CC7)C6)C5)CC4)CC3)no2)C(C)C)cc1]

Compound 55 was prepared using analogous procedures.

### Exemplary Synthesis of Compound 29

### Step 1

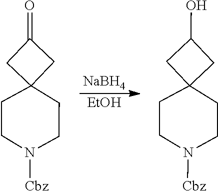

To a stirred solution of NaBH₄ (76.13 mg, 2.01 mmol, 1.1 eq) in EtOH (5 mL) was added a solution of benzyl 2-oxo-7-azaspiro[3.5]nonane-7-carboxylate (500 mg, 1.83 mmol, 1.0 eq) in EtOH (5 mL) at 0° C., and the reaction mixture was stirred at 0° C. for 1 hour. The reaction mixture was quenched with saturated aqueous NH₄C1 (2 mL) and concentrated under reduced pressure to remove EtOH. The residue was diluted with water (20 mL) and was extracted with EtOAc (20 mL×3). The combined organic layer was dried over anhydrous Na₂SO₄, filtered and concentrated under reduced pressure to give crude benzyl 2-hydroxy-7-azaspiro[3.5]nonane-7-carboxylate (488 mg, 1.77 mmol) as a gray oil, which was used directly in the next step without further purification.

### Step 2

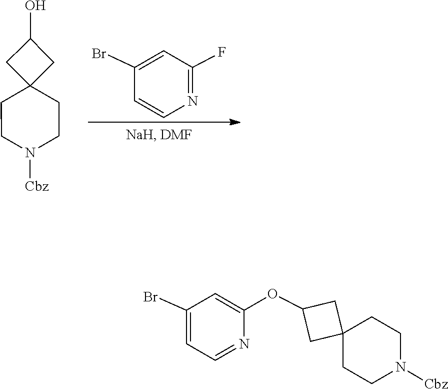 [SMILES: O=C(OCc1ccccc1)N1CCC2(CC1)CC(O)C2 ; Fc1cc(Br)ccn1 ; O=C(OCc1ccccc1)N1CCC2(CC1)CC(Oc1cc(Br)ccn1)C2]

To a solution of benzyl 2-hydroxy-7-azaspiro[3.5]nonane-7-carboxylate (388 mg, 1.41 mmol, 1.0 eq) in DMF (5 mL) was added NaH (68 mg, 60% dispersion in oil, 1.69 mmol, 1.2 eq) at 0° C. The mixture was stirred at 0° C. for 10 minutes under N₂. Then 4-bromo-2-fluoro-pyridine (247.99 mg, 1.41 mmol, 1.0 eq) was added. The mixture was warmed to 20° C. and stirred at 20° C. for 3 hours under N₂. The reaction mixture was poured into water (20 mL) and extracted with EtOAc (20 mL×3). The combined organic layer was washed with brine (20 mL×3), dried over anhydrous Na₂SO₄, filtered and concentrated under reduced pressure to give crude product, which was purified by flash silica gel chromatography (ISCO®; 20 g SepaFlash® SilicaFlash Column, eluent 0-15% ethyl acetate/petroleum ether @ 55 mL/min) to give benzyl 2-[(4-bromo-2-pyridyl)oxy]-7-azaspiro[3.5]nonane-7-carboxylate (584 mg, 1.35 mmol) as a yellow oil.

Benzyl 2-[(4-bromo-2-pyridyl)oxy]-7-azaspiro[3.5]nonane-7-carboxylate was converted to 2-(5-(8-(2-((7-azaspiro[3.5]nonan-2-yl)oxy)pyridin-4-yl)-3,8-diazabicyclo[3.2.1]octan-3-yl)-6-aminopyridazin-3-yl)phenol as shown in the scheme below using procedures analogous to those described for Exemplary Compound 13.

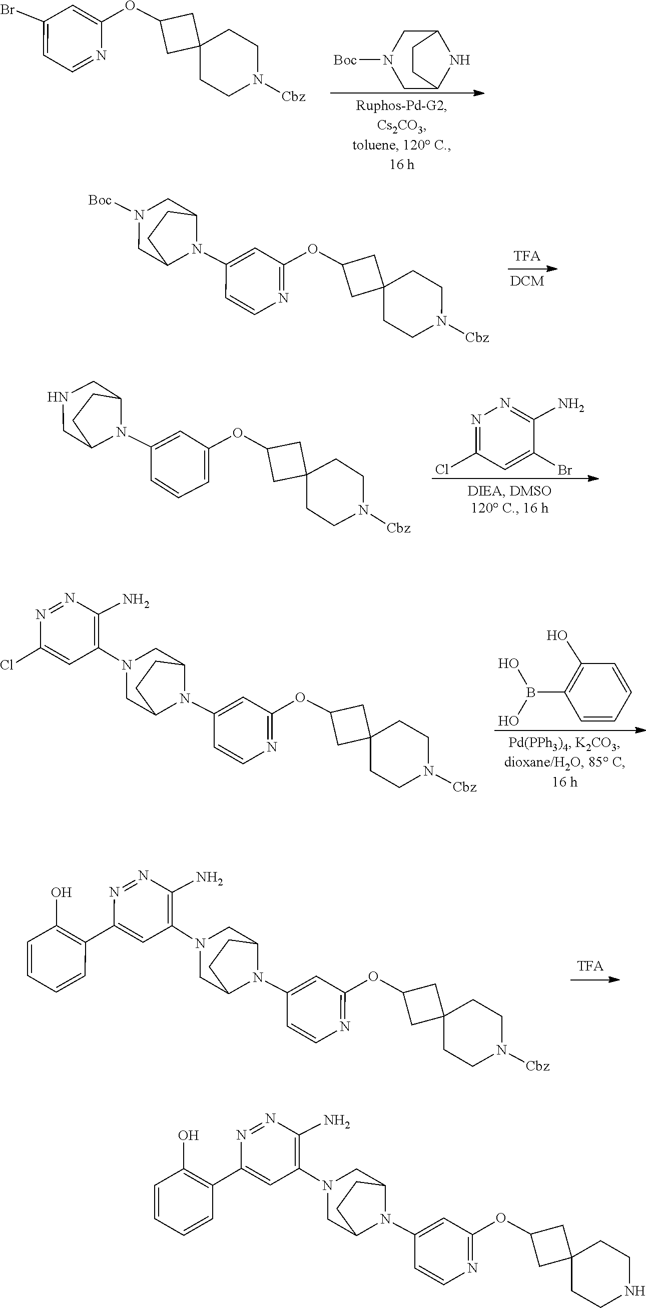

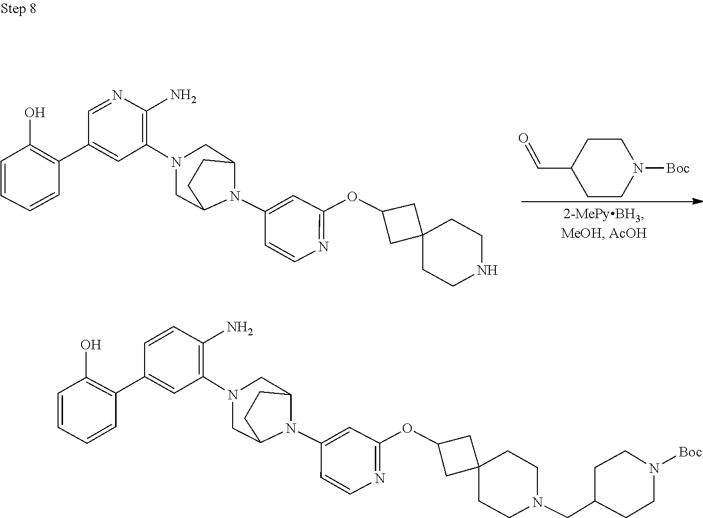 [SMILES: Nc1ncc(-c2ccccc2O)cc1N1CC2CCC(C1)N2c1ccnc(OC2CC3(CCNCC3)C2)c1 ; CC(C)(C)OC(=O)N1CCC(C=O)CC1 ; CC(C)(C)OC(=O)N1CCC(CN2CCC3(CC2)CC(Oc2cc(N4C5CCC4CN(c4cc(-c6ccccc6O)ccc4N)C5)ccn2)C3)CC1]

To a solution of 2-[6-amino-5-[8-[2-(7-azaspiro[3.5]nonan-2-yloxy)-4-pyridyl]-3,8-diazabicyclo[3.2.1]octan-3-yl]pyridazin-3-yl]phenol trifluoroacetate (285 mg, 454.07 umol, 1.0 eq) and tert-butyl 4-formylpiperidine-1-carboxylate (96.84 mg, 454.07 umol, 1.0 eq) in methanol (3.0 mL) and acetic acid (0.3 mL) was added 2-methylpyridine borane (242.84 mg, 2.27 mmol, 5.0 eq). The mixture was stirred at 20° C. for 16 hours. The reaction mixture was concentrated under reduced pressure. To the residue was added water (15 mL) and sat. aq. NaHCO₃ (10 mL), and the solution was extracted with EtOAc (25 mL×3). The combined organic layers were dried over anhydrous Na₂SO₄, filtered and concentrated under reduced pressure to give crude product, which was purified by silica gel chromatography (10% methanol/dichloromethane) to give tert-butyl 4-[[2-[[4-[3-[3-amino-6-(2-hydroxyphenyl)pyridazin-4-yl]-3,8-diazabicyclo[3.2.1]octan-8-yl]-2-pyridyl]oxy]-7-azaspiro[3.5]nonan-7-yl]methyl]piperidine-1-carboxylate (163 mg, 0.23 mmol) as a yellow solid.

tert-butyl 4-[[2-[[4-[3-[3-amino-6-(2-hydroxyphenyl)pyridazin-4-yl]-3,8-diazabicyclo[3.2.1]octan-8-yl]-2-pyridyl]oxy]-7-azaspiro[3.5]nonan-7-yl]methyl]piperidine-1-carboxylate was converted to the title compound as shown in the scheme below.

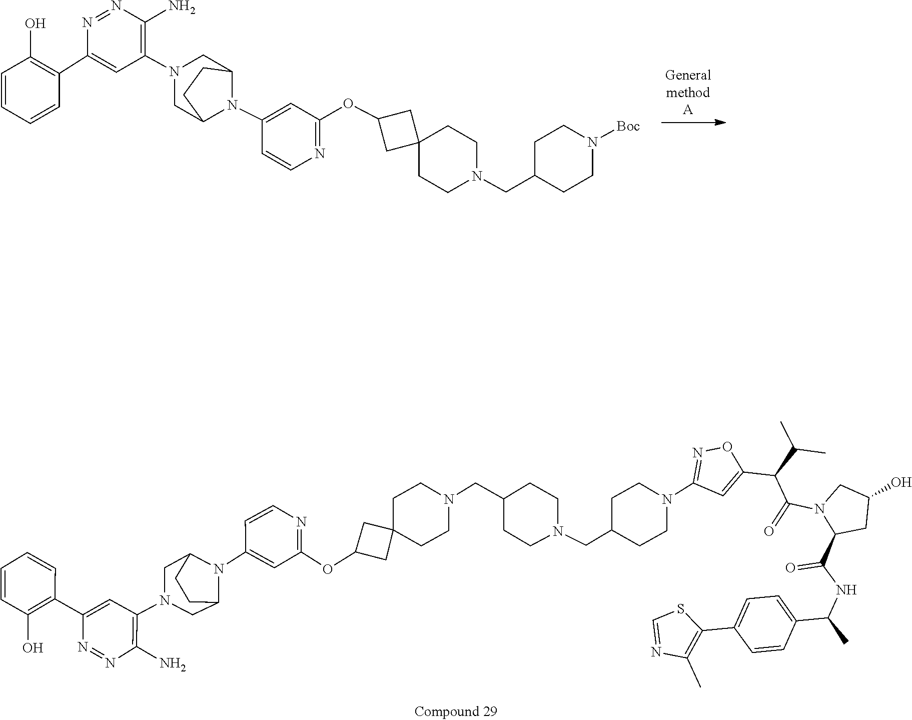 [SMILES: CC(C)(C)OC(=O)N1CCC(CN2CCC3(CC2)CC(Oc2cc(N4C5CCC4CN(c4cc(-c6ccccc6O)nnc4N)C5)ccn2)C3)CC1 ; Cc1ncsc1-c1ccc([C@H](C)NC(=O)[C@@H]2C[C@@H](O)CN2C(=O)[C@@H](c2cc(N3CCC(CN4CCC(CN5CCC6(CC5)CC(Oc5cc(N7C8CCC7CN(c7cc(-c9ccccc9O)nnc7N)C8)ccn5)C6)CC4)CC3)no2)C(C)C)cc1]

### Exemplary Synthesis of Compound 30

### Step 1

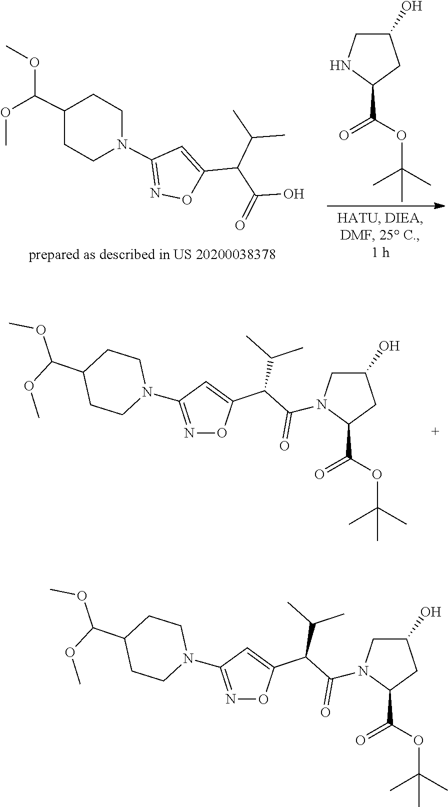

To a solution of 2-[3-[4-(dimethoxymethyl)-1-piperidyl]isoxazol-5-yl]-3-methyl-butanoic acid (3.8 g, 11.64 mmol, 1 eq), tert-butyl (2S,4R)-4-hydroxypyrrolidine-2-carboxylate (3.27 g, 17.46 mmol, 1.5 eq) in N,N-dimethylformamide (10 mL) was added 0-(7-azabenzotriazol-1-yl)-N,N,N′,N′-tetramethyluronium hexafluorophosphate (6.64 g, 17.46 mmol, 1.5 eq) and N,N—The reaction mixture was diluted with water (100 mL) and extracted with ethyl acetate (50 mL×3). The combined organic layers were washed with brine 30 mL, dried over anhydrous sodium sulfate, filtered and concentrated under reduced pressure to give a residue. The residue was purified by prep-HPLC (column: Phenomenex luna C18 250*80 mm*10 um; mobile phase: [water (0.225% FA)-ACN]; B %: 30%-55%, 20 min). Compound tert-butyl (2S,4R)-1-[(2S)-2-[3-[4-(dimethoxymethyl)-1-piperidyl]isoxazol-5-yl]-3-methyl-butanoyl]-4-hydroxy-pyrrolidine-2-carboxylate (910 mg, 1.84 mmol) was obtained as a yellow oil. Compound tert-butyl (2R)-1-[(2R)-2-[3-[4-(dimethoxymethyl)-1-piperidyl]isoxazol-5-yl]-3-methyl-butanoyl]-4-hydroxy-pyrrolidine-2-carboxylate (820 mg, 1.65 mmol) was obtained as a yellow solid.

### Step 2

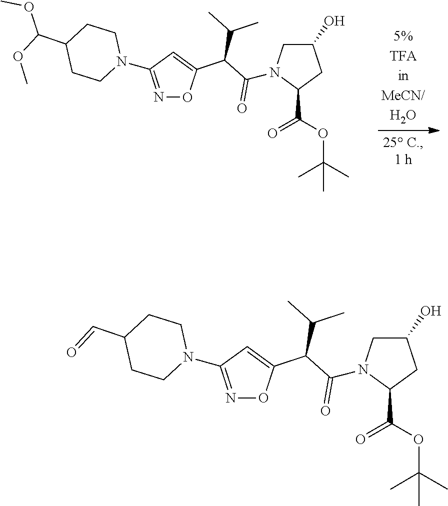 [SMILES: COC(OC)C1CCN(c2cc([C@H](C(=O)N3C[C@H](O)C[C@H]3C(=O)OC(C)(C)C)C(C)C)on2)CC1 ; CC(C)[C@@H](C(=O)N1C[C@H](O)C[C@H]1C(=O)OC(C)(C)C)c1cc(N2CCC(C=O)CC2)no1]

To a solution of tert-butyl (2S,4R)-1-[(2R)-2-[3-[4-(dimethoxymethyl)-1-piperidyl]isoxazol-5-yl]-3-methyl-butanoyl]-4-hydroxy-pyrrolidine-2-carboxylate (100 mg, 0.20 mmol, 1 eq) in acetonitrile (2.5 mL) and water (2.5 mL) was added trifluoroacetic acid (385 mg, 3.38 mmol, 16.73 eq). The mixture was stirred at 25° C. for 1 hours. The reaction mixture was diluted with saturation sodium hydrogen carbonate solution (10 mL) and extracted with ethyl acetate (20 mL×3). The combined organic layers were washed with brine (30 mL), dried over anhydrous sodium sulfate, filtered and concentrated under reduced pressure to give a residue. Compound tert-butyl (2S,4R)-1-[(2R)-2-[3-(4-formyl-1-piperidyl)isoxazol-5-yl]-3-methyl-butanoyl]-4-hydroxy-pyrrolidine-2-carboxylate (90 mg, 0.20 mmol) was obtained as a white solid.

tert-butyl (2S,4R)-1-[(2R)-2-[3-(4-formyl-1-piperidyl)isoxazol-5-yl]-3-methyl-butanoyl]-4-hydroxy-pyrrolidine-2-carboxylate was converted to the title compound as described in the scheme below.

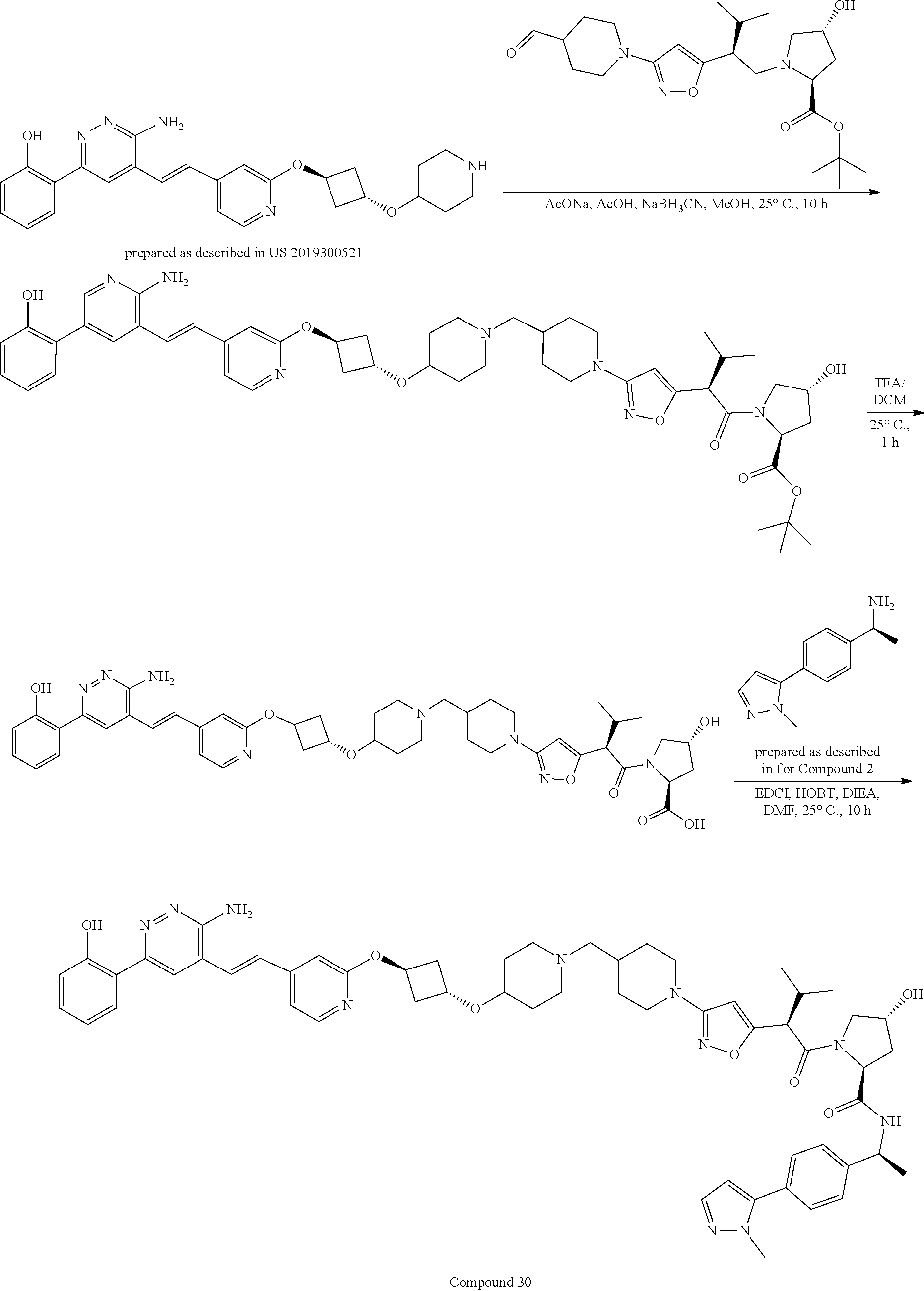

### Synthesis of Compound 31

Compound 31 was prepared according to the scheme below using procedures described as above as well as those commonly known to skilled in the art.

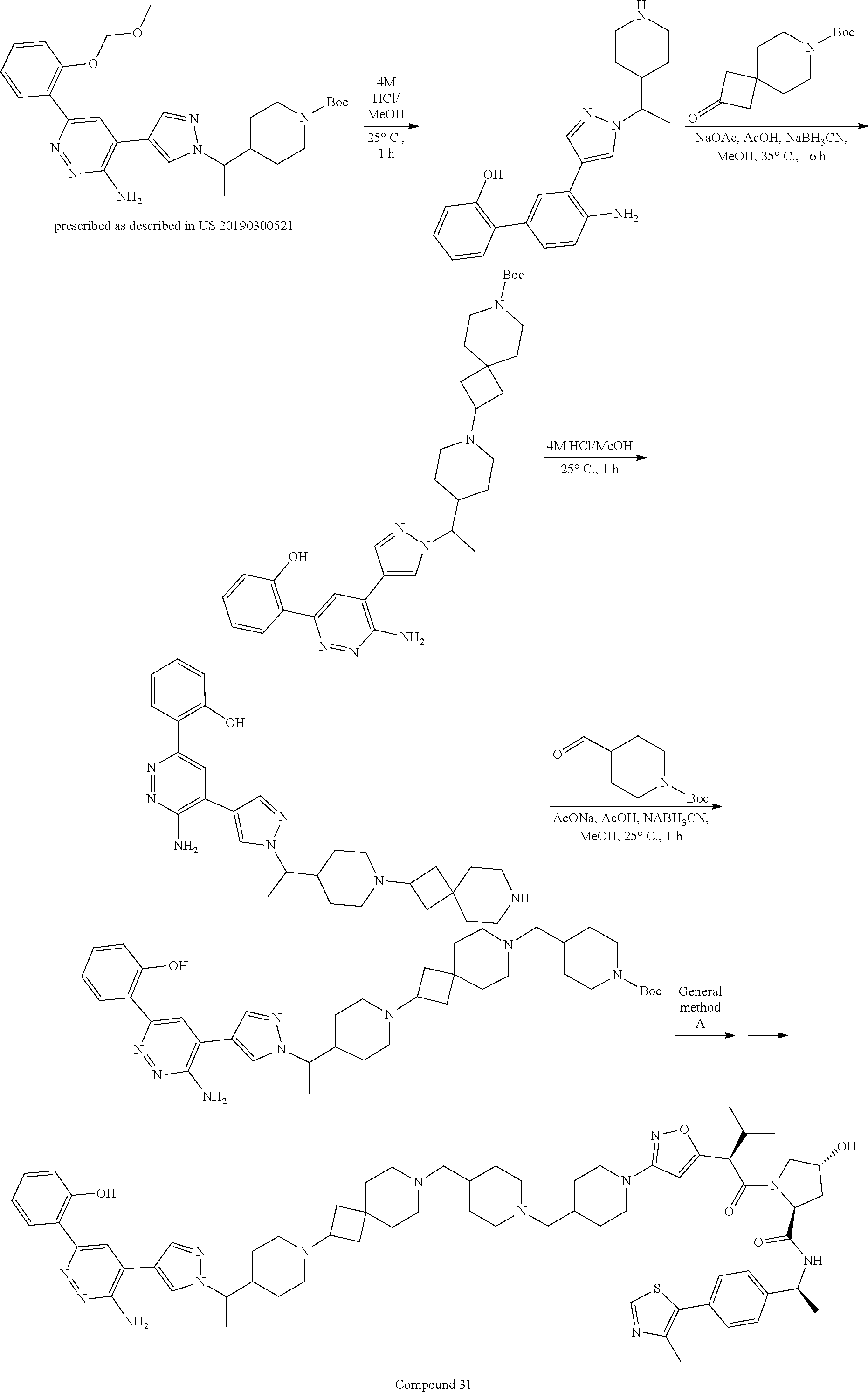

### Exemplary Synthesis of Compound 34

### Step 1

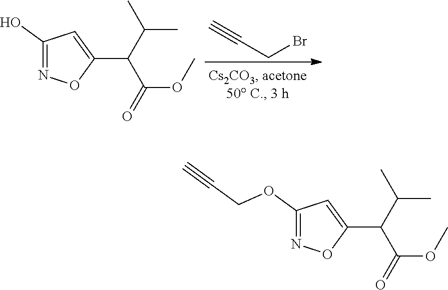

A mixture of methyl 2-(3-hydroxyisoxazol-5-yl)-3-methyl-butanoate (0.5 g, 2.51 mmol, 1 eq), 3-bromoprop-1-yne (1.12 g, 7.53 mmol, 0.8 mL, 3 eq) and cesium carbonate (1.64 g, 5.02 mmol, 2 eq) in acetone (15 mL) was stirred at 50° C. for 3 hours under nitrogen. The reaction mixture was filtered and concentrated under reduced pressure to give a residue. The residue was purified by silica gel column chromatography (petroleum ether/ethyl acetate=5/1). Compound methyl 3-methyl-2-(3-prop-2-ynoxyisoxazol-5-yl)butanoate (460 mg, 1.94 mmol) was obtained as a light yellow oil.

### Step 2

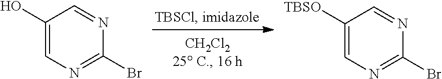 [SMILES: Oc1cnc(Br)nc1 ; CC(C)(C)[Si](C)(C)Oc1cnc(Br)nc1]

To a solution of 2-bromopyrimidin-5-ol (3 g, 17.14 mmol, 1 eq) in dichloromethane (70 mL) was added imidazole (1.75 g, 25.72 mmol, 1.5 eq) and tert-butylchlorodimethylsilane (3.88 g, 25.72 mmol, 3.2 mL, 1.5 eq) at 25° C. The mixture was stirred at 25° C. for 16 hours. The reaction mixture was diluted with dichloromethane (100 mL), washed with water (20 mL×2) and brine (20 mL), dried with anhydrous sodium sulfate, filtered and concentrated under reduced pressure to give a residue. The residue was purified by silica gel column chromatography (petroleum ether/ethyl acetate=10/1). Compound (2-bromopyrimidin-5-yl)oxy-tert-butyl-dimethyl-silane (4.7 g, 16.25 mmol) was obtained as a colorless oil.

### Step 3

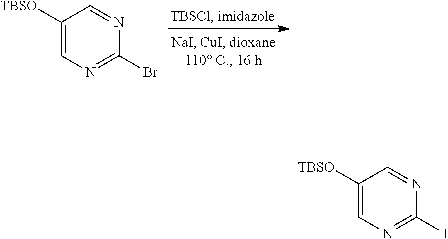 [SMILES: CC(C)(C)[Si](C)(C)Oc1cnc(Br)nc1 ; CC(C)(C)[Si](C)(C)Oc1cnc(I)nc1]

A mixture of (2-bromopyrimidin-5-yl)oxy-tert-butyl-dimethyl-silane (2.7 g, 9.33 mmol, 1 eq), sodium iodide (7.0 g, 46.67 mmol, 5 eq), cuprous iodide (178 mg, 0.93 mmol, 0.1 eq) and N,N′-dimethylethane-1,2-diamine (82 mg, 0.93 mmol, 0.1 mL, 0.1 eq) in dioxane (40 mL) was stirred at 110° C. for 16 hours under nitrogen. The reaction mixture was filtered and concentrated under reduced pressure to give a residue. The residue was purified by silica gel column chromatography (petroleum ether/ethyl acetate=10/1 to 2/1). Compound tert-butyl-(2-iodopyrimidin-5-yl)oxy-dimethyl-silane (1.7 g, 5.06 mmol) was obtained as a white solid.

### Step 4

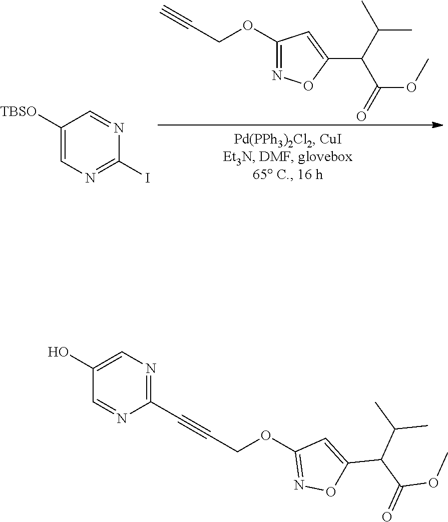 [SMILES: CC(C)(C)[Si](C)(C)Oc1cnc(I)nc1 ; C#CCOc1cc(C(C(=O)OC)C(C)C)on1 ; COC(=O)C(c1cc(OCC#Cc2ncc(O)cn2)no1)C(C)C]

A mixture of methyl 3-methyl-2-(3-prop-2-ynoxyisoxazol-5-yl)butanoate (200 mg, 0.84 mmol, 1 eq), tert-butyl-(2-iodopyrimidin-5-yl)oxy-dimethyl-silane (340 mg, 1.01 mmol, 1.2 eq), bis(triphenylphosphine)palladium(II) dichloride (59 mg, 0.08 mmol, 0.1 eq), cuprous iodide (16 mg, 0.08 mmol, 0.1 eq) and triethylamine (256 mg, 2.53 mmol, 0.4 mL, 3 eq) in N,N-dimethylformamide (6 mL) was stirred at 65° C. for 16 hours under nitrogen (glovebox). The reaction mixture was filtered and concentrated under reduced pressure to give a residue. The residue was purified by preparative HPLC (column: Phenomenex luna C18 150*40 mm*15 um; mobile phase: [water (0.225% FA)-ACN]; B %: 68%-95%, 11 min). Compound methyl 2-[3-[3-(5-hydroxypyrimidin-2-yl)prop-2-ynoxy]isoxazol-5-yl]-3-methyl-butanoate (240 mg, 0.72 mmol) was obtained as a brown oil.

(1s, 3s)-3-((4-(4-(3-amino-6-(2-(methoxymethoxy)phenyl)pyridazin-4-yl)piperazin-1-yl)pyridin-2-yl)oxy)cyclobutan-1-ol was prepared according to the scheme below using procedures described in Exemplary Compounds 13 and 6 above, as well as those commonly known to skilled in the art.

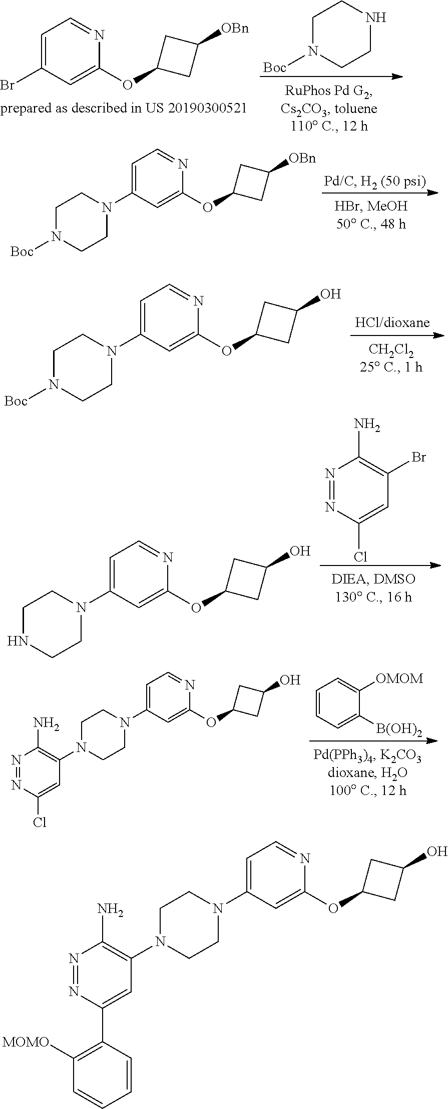

(1s, 3s)-3-((4-(4-(3-amino-6-(2-(methoxymethoxy)phenyl)pyridazin-4-yl)piperazin-1-yl)pyridin-2-yl)oxy)cyclobutan-1-ol was converted to the title compound according to the scheme below using procedures commonly known to those skilled in art.

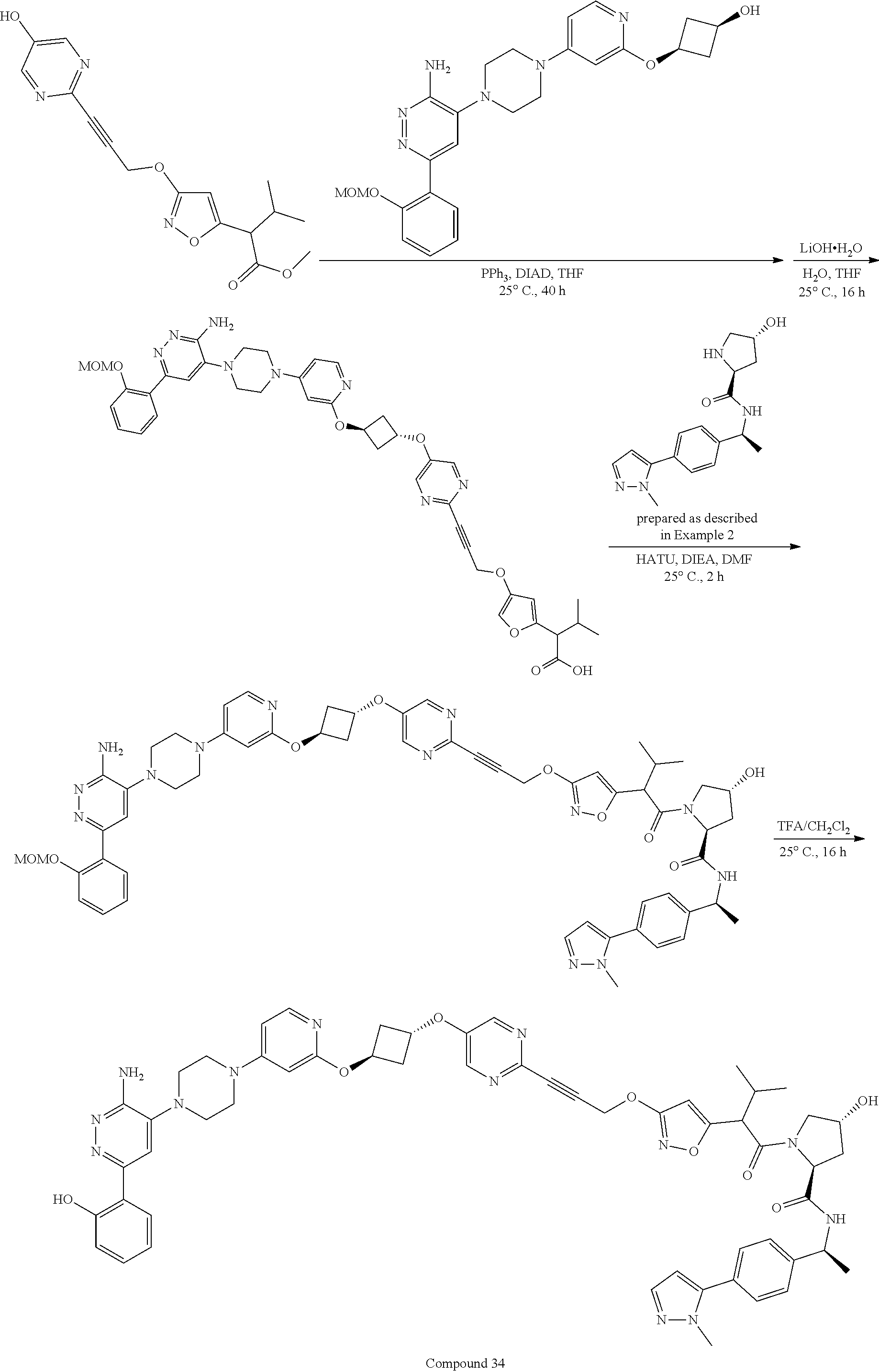

### Exemplary Synthesis of Compound 35

Compound 35 was prepared according to the scheme below using procedures described above, as well as general procedures known to those skilled in the art.

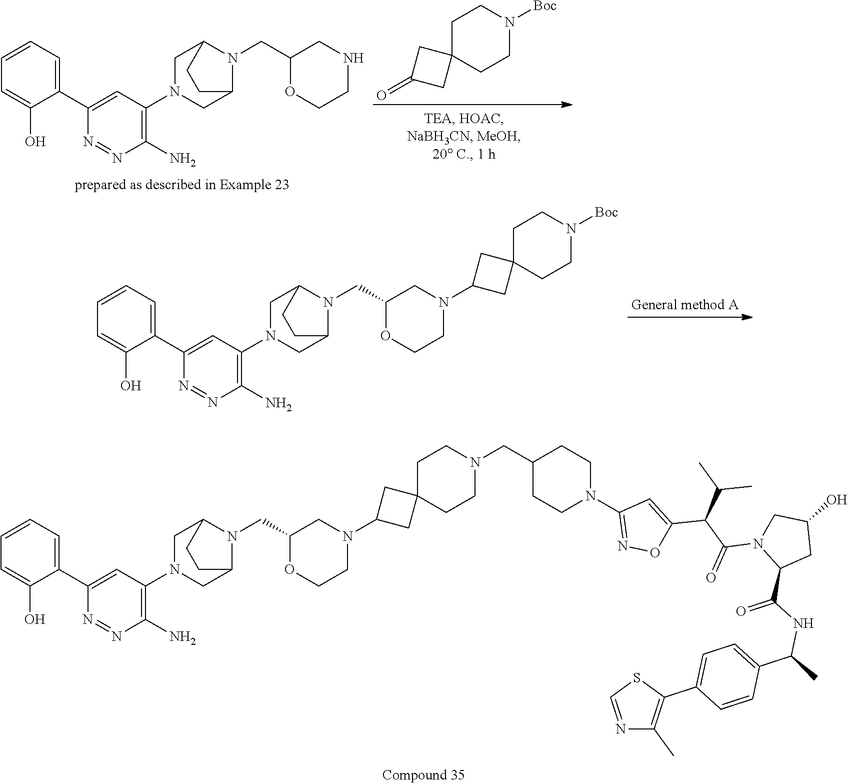

Compounds 73, 92, 106, 113, 141, 142, and 143 were prepared using analogous procedures.

### Exemplary Synthesis of Compound 36

### Step 1

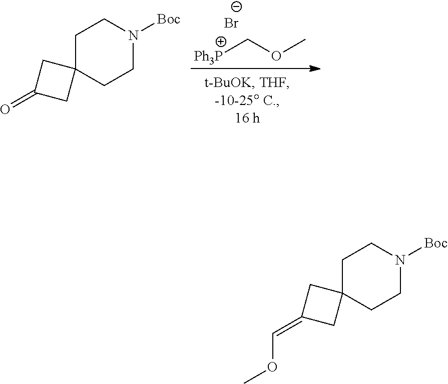

To a solution of methoxymethyl(triphenyl)phosphonium chloride (15.76 g, 45.97 mmol, 2.2 eq) in tetrahydrofuran (100 mL) was added potassium tert-butoxide (1 M, 41.8 mL, 2 eq) at −10° C. under nitrogen. The mixture was stirred at −10° C. for 1 hour. Then tert-butyl 2-oxo-7-azaspiro[3.5]nonane-7-carboxylate (5 g, 20.89 mmol, 1 eq) in tetrahydrofuran (30 mL) was added at −10° C. The mixture was warmed to 25° C. and stirred for 15 hours. The reaction mixture was quenched by addition of the saturated ammonium chloride solution (30 mL) at 25° C., and then diluted with water 20 mL and extracted with ethyl acetate (100 mL×3). The combined organic layers were washed with brine (50 mL), dried with anhydrous sodium sulfate, filtered and concentrated under reduced pressure to give a residue. The residue was purified by silica gel chromatography (petroleum ether/ethyl acetate=10/1 to 5:1). Compound tert-butyl 2-(methoxymethylene)-7-azaspiro[3.5]nonane-7-carboxylate (4.5 g, 16.83 mmol) was obtained as a colorless oil.

### Step 2

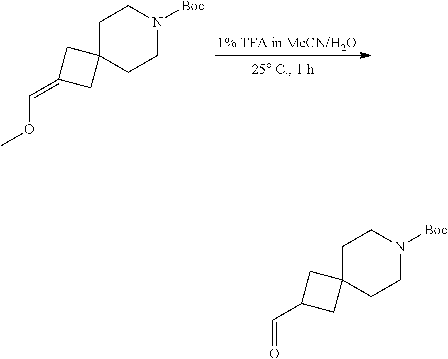 [SMILES: COC=C1CC2(CCN(C(=O)OC(C)(C)C)CC2)C1 ; CC(C)(C)OC(=O)N1CCC2(CC1)CC(C=O)C2]

To a solution of trifluoroacetic acid (539 mg, 4.73 mmol, 1.26 eq) in acetonitrile (36 mL) and water (9 mL) was added tert-butyl 2-(methoxymethylene)-7-azaspiro[3.5]nonane-7-carboxylate (1 g, 3.74 mmol, 1 eq). The resulting mixture was stirred at 25° C. for 1 hour. The mixture was added to the saturated sodium bicarbonate (100 mL), and the mixture was extracted with ethyl acetate (80 mL×3). The combined organic phase was washed with brine (30 mL), dried over sodium sulfate, filtered and concentrated in vacuum. The residue was purified by silica gel chromatography (petroleum ether/ethyl acetate=50/1 to 10:1). Compound tert-butyl 2-formyl-7-azaspiro [3.5]nonane-7-carboxylate (700 mg, 2.76 mmol) was obtained as a colorless oil.

### Step 3

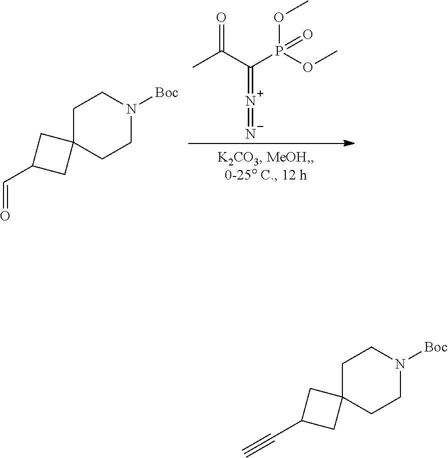

To a solution of tert-butyl 2-formyl-7-azaspiro[3.5]nonane-7-carboxylate (700 mg, 2.76 mmol, 1 eq) in methanol (7 mL) was added 1-diazo-1-dimethoxyphosphoryl-propan-2-one (637 mg, 3.32 mmol, 1.2 eq) and potassium carbonate (764 mg, 5.53 mmol, 2 eq) at 0° C. The mixture was stirred at 25° C. for 12 hours. 50 mL water was added to the mixture, and the mixture was extracted with ethyl acetate (50 mL×3). The combined organic phase was washed with brine (30 mL×2), dried with anhydrous sodium sulfate, filtered and concentrated in vacuum. The residue was purified by silica gel chromatography (petroleum ether/ethyl acetate=100:1 to 20:1). Compound tert-butyl 2-ethynyl-7-azaspiro[3.5]nonane-7-carboxylate (500 mg, 2.01 mmol) was obtained as a colorless oil.

### Step 4

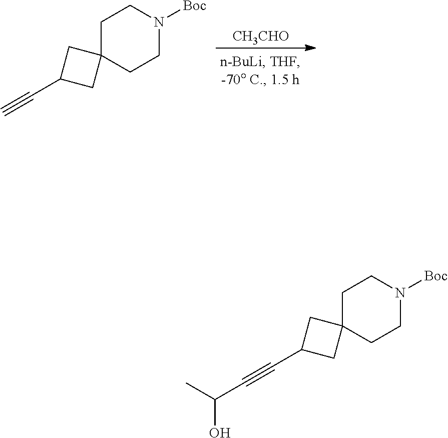

To a solution of tert-butyl 2-ethynyl-7-azaspiro[3.5]nonane-7-carboxylate (500 mg, 2.01 mmol, 1 eq) in tetrahydrofuran (10 mL) was added n-butyllithium (2.5 M, 1.6 mL, 2 eq) at −70° C. The mixture was stirred at −70° C. for 0.5 hours. Then acetaldehyde (265 mg, 6.02 mmol, 3 eq) was added to the mixture. The reaction mixture was stirred at −70° C. for 1 hour. 50 mL water was added to the mixture, then the mixture extracted with ethyl acetate (50 mL×3). The combined organic phase was washed with brine (30 mL×2), dried with anhydrous sodium sulfate, filtered and concentrated in vacuum. The residue was purified by silica gel chromatography (petroleum ether/ethyl acetate=50:1 to 5:1). Compound tert-butyl 2-(3-hydroxybut-1-ynyl)-7-azaspiro[3.5]nonane-7-carboxylate (400 mg, 1.36 mmol) was obtained as a yellow oil.

### Step 5

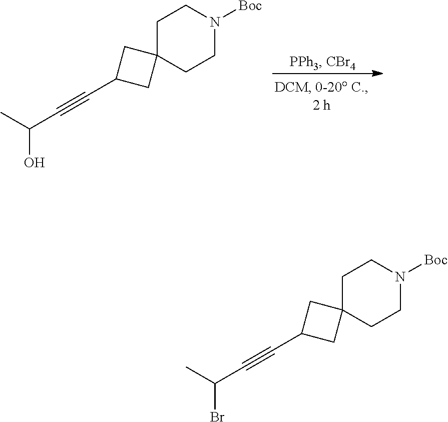

To a solution of tert-butyl 2-(3-hydroxybut-1-ynyl)-7-azaspiro[3.5]nonane-7-carboxylate (400 mg, 1.36 mmol, 1 eq) in dichloromethane (5 mL) was added triphenylphosphine (429 mg, 1.64 mmol, 1.2 eq) and tetrabromomethane (543 mg, 1.64 mmol, 1.2 eq) at 0° C. The reaction mixture was stirred at 20° C. for 2 hour. The reaction mixture was concentrated under reduced pressure to give a residue. The residue was purified by silica gel chromatography (petroleum ether/ethyl acetate=20:1 to 10:1). Compound tert-butyl 2-(3-bromobut-1-ynyl)-7-azaspiro[3.5]nonane-7-carboxylate (233 mg, 0.65 mmol) was obtained as a white solid.

### Step 6

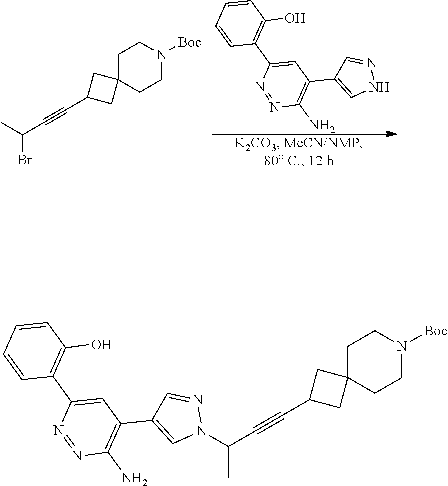

A mixture of tert-butyl 2-(3-bromobut-1-ynyl)-7-azaspiro[3.5]nonane-7-carboxylate (223 mg, 0.62 mmol, 1 eq), 6-[2-(methoxymethoxy)phenyl]-4-(1H-pyrazol-4-yl)pyridazin-3-amine (186 mg, 0.62 mmol, 1 eq), potassium carbonate (259 mg, 1.88 mmol, 3 eq) in acetonitrile (3 mL) and 1-methyl-2-pyrrolidinone (1 mL) was degassed and purged with nitrogen for 3 times, and then the mixture was stirred at 80° C. for 12 hours under nitrogen. 50 mL water was added to the mixture, and then the mixture extracted with ethyl acetate (50 mL×3). The combined organic phase was washed with brine (30 mL×2), dried with anhydrous sodium sulfate, filtered and concentrated in vacuum. The residue was purified by prep-HPLC (column: Phenomenex luna C18 150*25 mm*10 um; mobile phase: [water (0.1% TFA)-ACN]; B %: 40%-70%, 10 min). Compound tert-butyl 2-[3-[4-[3-amino-6-[2-(methoxymethoxy)phenyl]pyridazin-4-yl]pyrazol-1-yl]but-1-ynyl]-7-azaspiro[3.5]nonane-7-carboxylate (130 mg, 0.23 mmol) was obtained as a yellow oil.

tert-Butyl 2-[3-[4-[3-amino-6-[2-(methoxymethoxy)phenyl]pyridazin-4-yl]pyrazol-1-yl]but-1-ynyl]-7-azaspiro[3.5]nonane-7-carboxylate was converted to the title compound as shown in the scheme below.

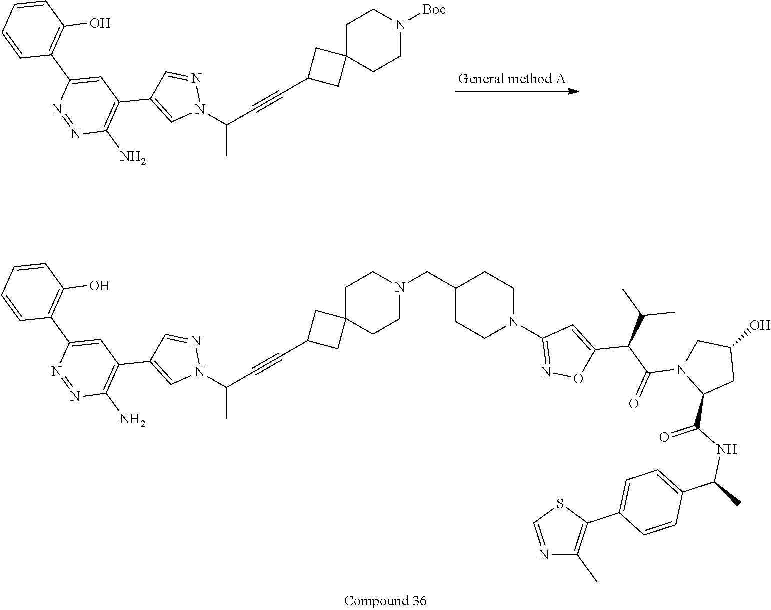 [SMILES: CC(C#CC1CC2(CCN(C(=O)OC(C)(C)C)CC2)C1)n1cc(-c2cc(-c3ccccc3O)nnc2N)cn1 ; Cc1ncsc1-c1ccc([C@H](C)NC(=O)[C@@H]2C[C@@H](O)CN2C(=O)[C@@H](c2cc(N3CCC(CN4CCC5(CC4)CC(C#CC(C)n4cc(-c6cc(-c7ccccc7O)nnc6N)cn4)C5)CC3)no2)C(C)C)cc1]

### Exemplary Synthesis of Compound 38

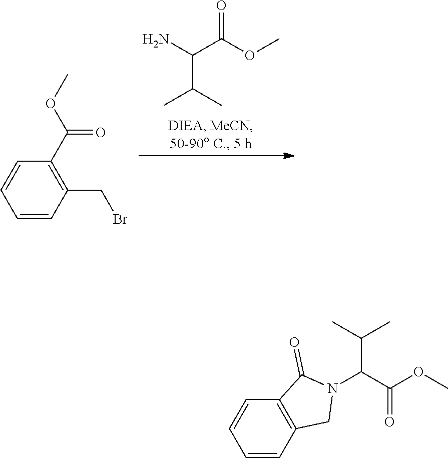

To a solution of methyl 2-(bromomethyl)benzoate (3.7 g, 16.15 mmol, 1 eq), and methyl 2-amino-3-methyl-butanoate (3.25 g, 19.38 mmol, 1.2 eq, hydrochloride) in acetonitrile (70 mL) was added N,N-diisopropylethylamine (10.44 g, 80.76 mmol, 14.1 mL, 5 eq). The reaction mixture was stirred at 50° C. for 2 hours and 90° C. for 3 hours. Water (100 mL) was added to the mixture. The aqueous phase was extracted with ethyl acetate (80 mL×3). The combined organic phase was washed with brine (100 mL), dried over sodium sulfate, filtered and concentrated in vacuum. The residue was purified by silica gel chromatography (petroleum ether: ethyl acetate=100:1 to 3:1). Methyl 3-methyl-2-(1-oxoisoindolin-2-yl)butanoate (3.4 g, 13.75 mmol) was obtained as a light yellow oil.

Methyl 3-methyl-2-(1-oxoisoindolin-2-yl)butanoate was converted to (2S,4R)-4-hydroxy-1-((S)-3-methyl-2-(1-oxoisoindolin-2-yl)butanoyl)pyrrolidine-2-carboxylic acid as described in the schemes below using procedures commonly known to those skilled in the art.

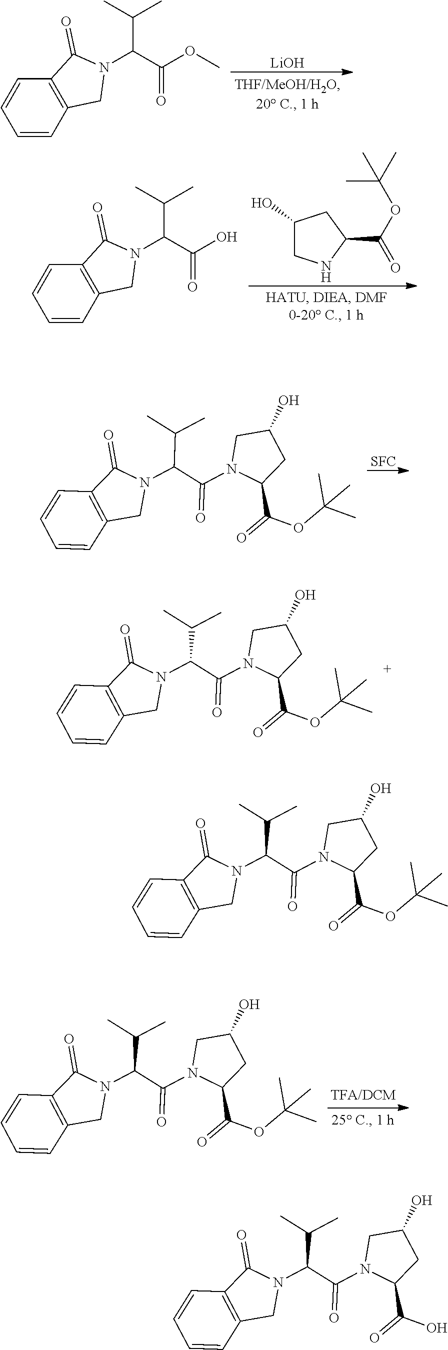

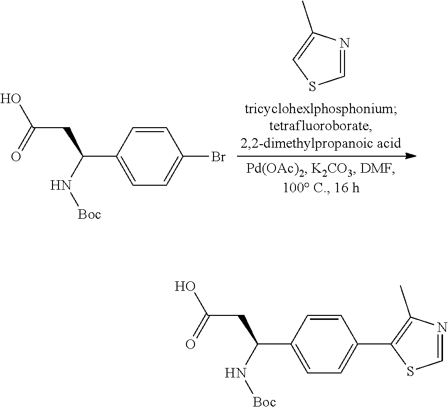

A mixture of (3S)-3-(4-bromophenyl)-3-(tert-butoxycarbonylamino)propanoic acid (1 g, 2.91 mmol, 1 eq), 4-methylthiazole (2.88 g, 29.05 mmol, 2.6 mL, 10 eq), palladium(II) acetate (65 mg, 0.29 mmol, 0.1 eq), potassium carbonate (602 mg, 4.36 mmol, 1.5 eq) tricyclohexylphosphonium tetrafluoroborate (106 mg, 0.29 mmol, 0.1 eq) and 2,2-dimethylpropanoic acid (89 mg, 0.87 mmol, 0.1 mL, 0.3 eq) in N,N-dimethylformamide (10 mL) was degassed and purged with nitrogen for 3 times, and then the mixture was stirred at 100° C. for 16 h under nitrogen atmosphere. 50 mL water was added to the mixture, and the mixture was extracted with ethyl acetate (50 mL×3). The combined organic phase was washed with brine (30 mL×2), dried with anhydrous sodium sulfate, filtered and concentrated in vacuum. The residue was purified by prep-HPLC (column: Phenomenex luna C18 250*50 mm*10 um; mobile phase: [water (0.225% FA)-ACN]; B %: 10%-50%, 22 min). Compound (3S)-3-(tert-butoxycarbonylamino)-3-[4-(4-methylthiazol-5-yl)phenyl]propanoic acid (400 mg, 1.10 mmol) was obtained as a yellow solid.

The title compound was obtained from 2-(6-amino-5-(8-(2-((1r, 3r)-3-(piperidin-4-yloxy)cyclobutoxy)pyridin-4-yl)-3,8-diazabicyclo[3.2.1]octan-3-yl)pyridazin-3-yl)phenol [prepared as described in US 20190300521] according to the scheme below.

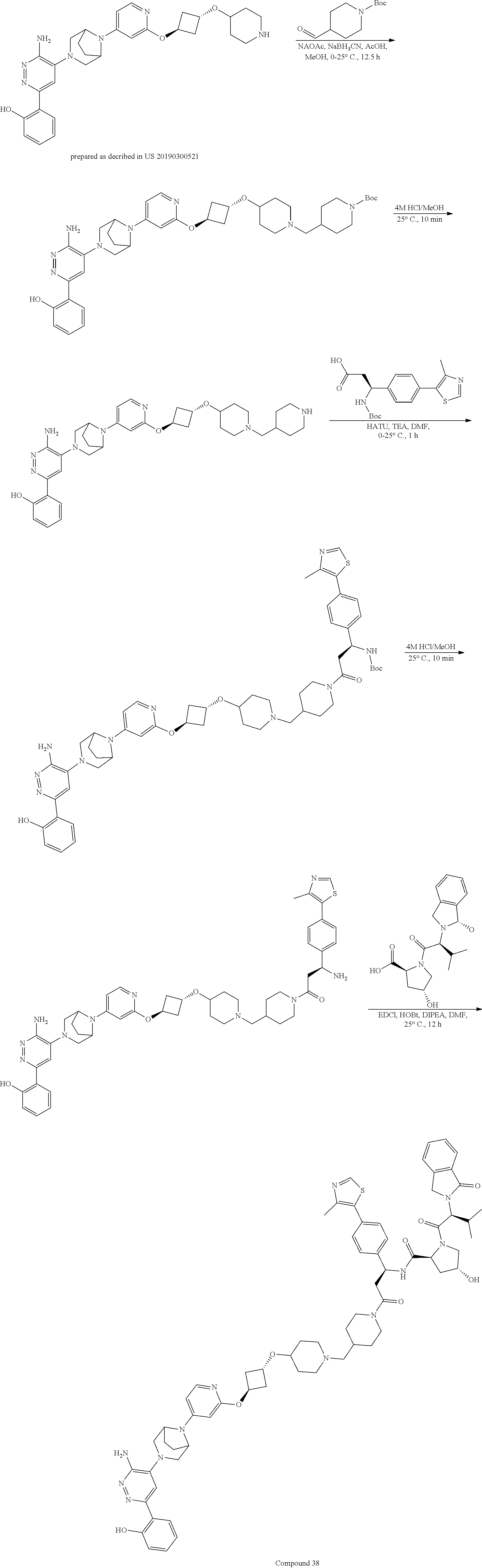

### Exemplary Synthesis of Compound 39

Compound 39 was prepared according to the scheme below using procedures analogous to those described for Compounds 2 and 29.

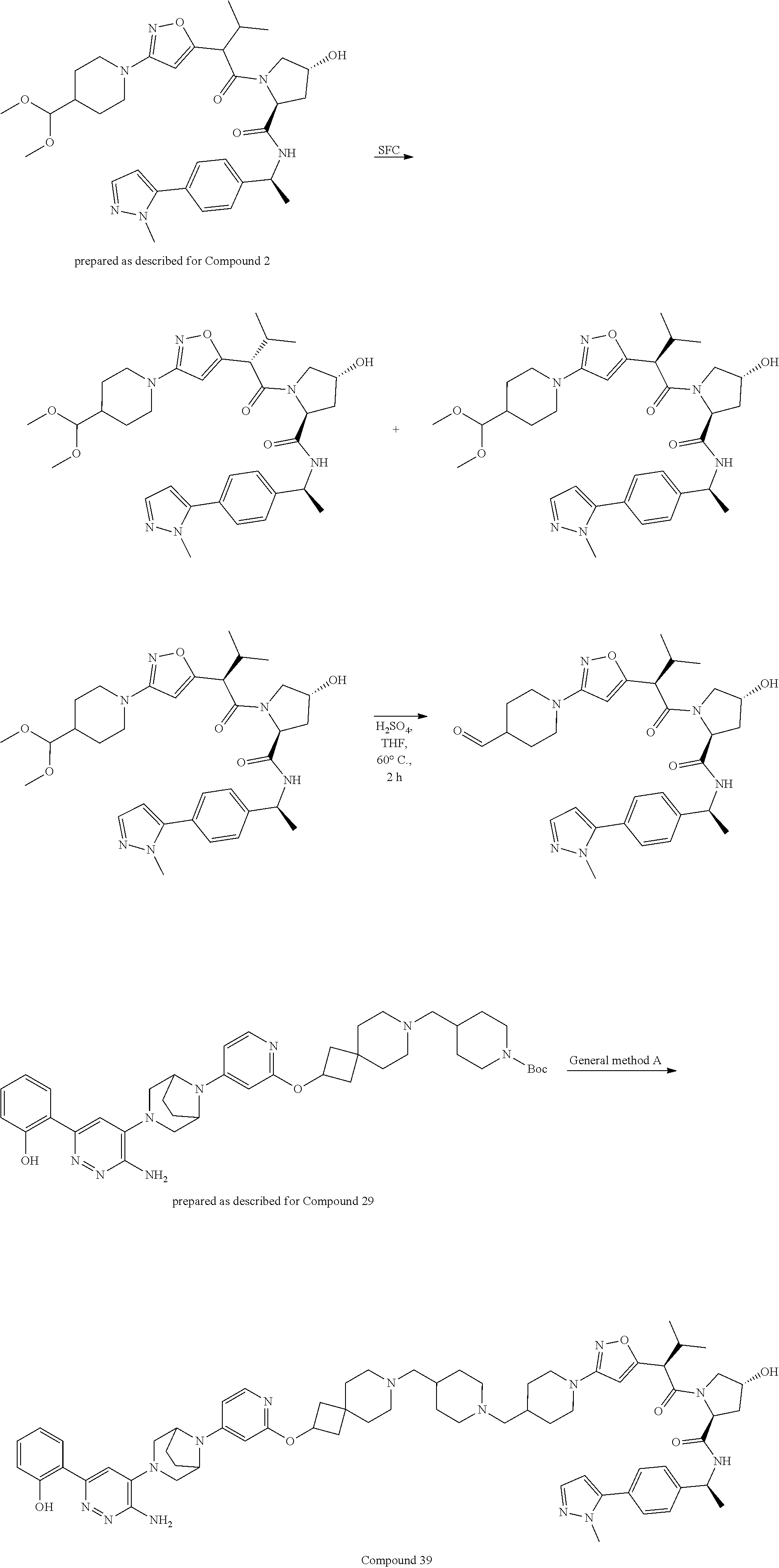

### Exemplary Synthesis of Compound 40

### Step 1

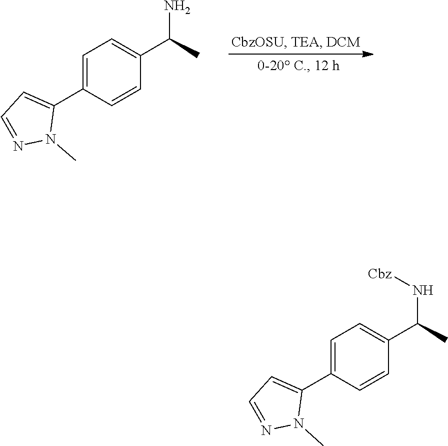 [SMILES: C[C@H](N)c1ccc(-c2ccnn2C)cc1 ; C[C@H](NC(=O)OCc1ccccc1)c1ccc(-c2ccnn2C)cc1]

To a solution of (1S)-1-[4-(2-methylpyrazol-3-yl)phenyl]ethanamine (1.6 g, 6.73 mmol, 1 eq, hydrochloride) and triethylamine (3.41 g, 33.65 mmol, 4.7 mL, 5 eq) in dichloromethane (25 mL) was added N-(benzyloxycarbonyloxy)succinimide (2.52 g, 10.10 mmol, 1.5 eq) at 0° C. The reaction solution was stirred at 20° C. for 12 hours. The reaction solution was concentrated under vacuum to remove solvents, diluted with water (30 mL) and extracted with ethyl acetate (30 mL×2). The combined organic layer was dried over anhydrous sodium sulfate, filtered and concentrated under vacuum to get the residue. The residue was purified by silica gel column chromatography (petroleum ether/ethyl acetate=10:1 to 2:1) to get benzyl N-[(1S)-1-[4-(2-methylpyrazol-3-yl)phenyl]ethyl]carbamate (2.1 g, 6.26 mmol) as a white solid.

### Step 2

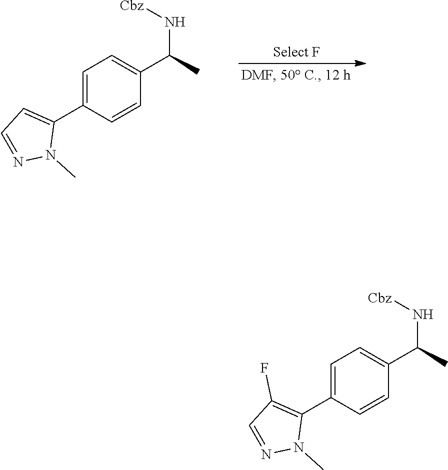 [SMILES: C[C@H](NC(=O)OCc1ccccc1)c1ccc(-c2ccnn2C)cc1 ; C[C@H](NC(=O)OCc1ccccc1)c1ccc(-c2c(F)cnn2C)cc1]

To a solution of benzyl N-[(1S)-1-[4-(2-methylpyrazol-3-yl)phenyl]ethyl]carbamate (220 mg, 0.66 mmol, 1 eq) in N,N-dimethylformamide (4 mL) was added Selectfluor® fluorinating reagent (302 mg, 0.85 mmol, 1.3 eq). The reaction solution was stirred at 50° C. for 12 hours. The reaction solution was cooled to 20° C. and was diluted with water (30 mL) and was extracted with ethyl acetate (20 mL×2). The combined organic layer was washed with brine (20 mL×4). The organic layer was dried over anhydrous sodium sulfate, filtered and concentrated under vacuum to get the residue. The residue was purified by prep-TLC (petroleum ether/ethyl acetate=1/1). Benzyl N-[(1S)-1-[4-(4-fluoro-2-methyl-pyrazol-3-yl)phenyl]ethyl] carbamate (260 mg, 0.74 mmol) was obtained as a colorless gum.

### Step 3

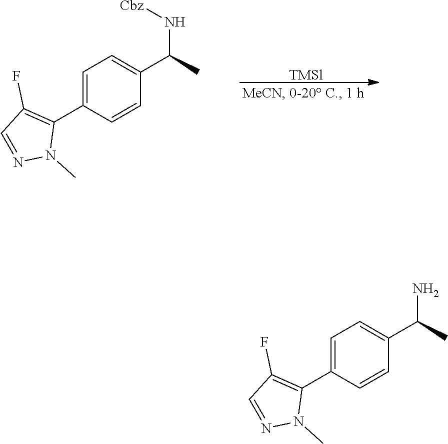 [SMILES: C[C@H](NC(=O)OCc1ccccc1)c1ccc(-c2c(F)cnn2C)cc1 ; "TMSl" ; C[C@H](N)c1ccc(-c2c(F)cnn2C)cc1]

To a solution of benzyl N-[(1S)-1-[4-(4-fluoro-2-methyl-pyrazol-3-yl)phenyl]ethyl]carbamate (260 mg, 0.74 mmol, 1 eq) in acetonitrile (5 mL) was added trimethyliodosilane (294 mg, 1.47 mmol, 0.2 mL, 2 eq) at 0° C., and the reaction solution was stirred at 20° C. for 1 hour. The reaction solution was quenched with methanol (8 mL) and concentrated under vacuum to get the residue. The residue was purified by prep-HPLC (column: Phenomenex luna C18 150*25 mm*10 um; mobile phase: [water (0.1% TFA)-ACN]; B %: 1%-31%, 10 min). (1S)-1-[4-(4-fluoro-2-methyl-pyrazol-3-yl)phenyl]ethanamine trifluoroacetate (168 mg, 0.50 mmol) was obtained as a light yellow solid.

### Step 4

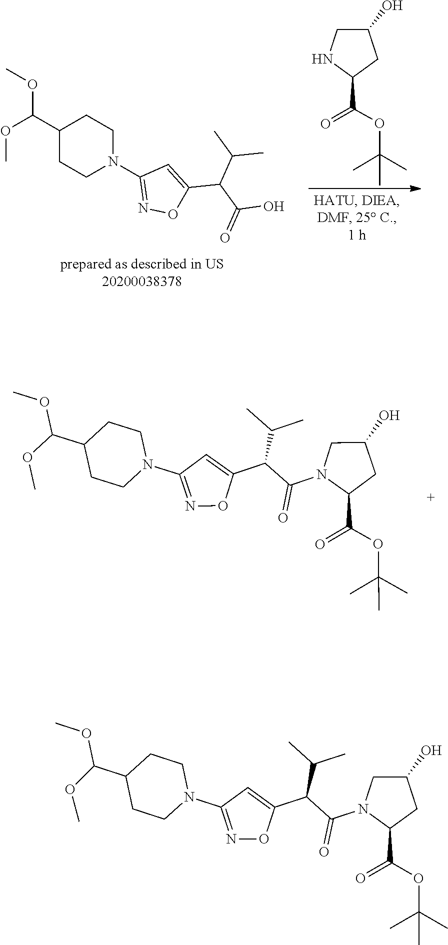

To a solution of 2-[3-[4-(dimethoxymethyl)-1-piperidyl]isoxazol-5-yl]-3-methyl-butanoic acid (3.8 g, 11.64 mmol, 1 eq), tert-butyl (2S,4R)-4-hydroxypyrrolidine-2-carboxylate (3.27 g, 17.46 mmol, 1.5 eq) in N,N-dimethylformamide (10 mL) was added 0-(7-azabenzotriazol-1-yl)-N,N,N′,N′-tetramethyluronium hexafluorophosphate (6.64 g, 17.46 mmol, 1.5 eq) and N,N-diisopropylethylamine (4.51 g, 34.93 mmol, 6.1 mL, 3 eq). The mixture was stirred at 25° C. for 1 hour. The reaction mixture was diluted with water (100 mL) and extracted with ethyl acetate (50 mL×3). The combined organic layers were washed with brine 30 mL, dried over anhydrous sodium sulfate, filtered and concentrated under reduced pressure to give a residue. The residue was purified by prep-HPLC (column: Phenomenex luna C18 250*80 mm*10 um; mobile phase: [water (0.225% FA)-ACN]; B %: 30%-60%, 25 min). Compound tert-butyl (2S,4R)-1-[(2S)-2-[3-[4-(dimethoxymethyl)-1-piperidyl]isoxazol-5-yl]-3-methyl-butanoyl]-4-hydroxy-pyrrolidine-2-carboxylate (910 mg, 1.84 mmol) was obtained as a yellow oil. Compound tert-butyl (2S,4R)-1-[(2R)-2-[3-[4-(dimethoxymethyl)-1-piperidyl]isoxazol-5-yl]-3-methyl-butanoyl]-4-hydroxy-pyrrolidine-2-carboxylate (820 mg, 1.65 mmol) was obtained as a yellow solid.

The title compound was prepared according to the scheme below using procedures analogous to those described for Compounds 2 and 4.

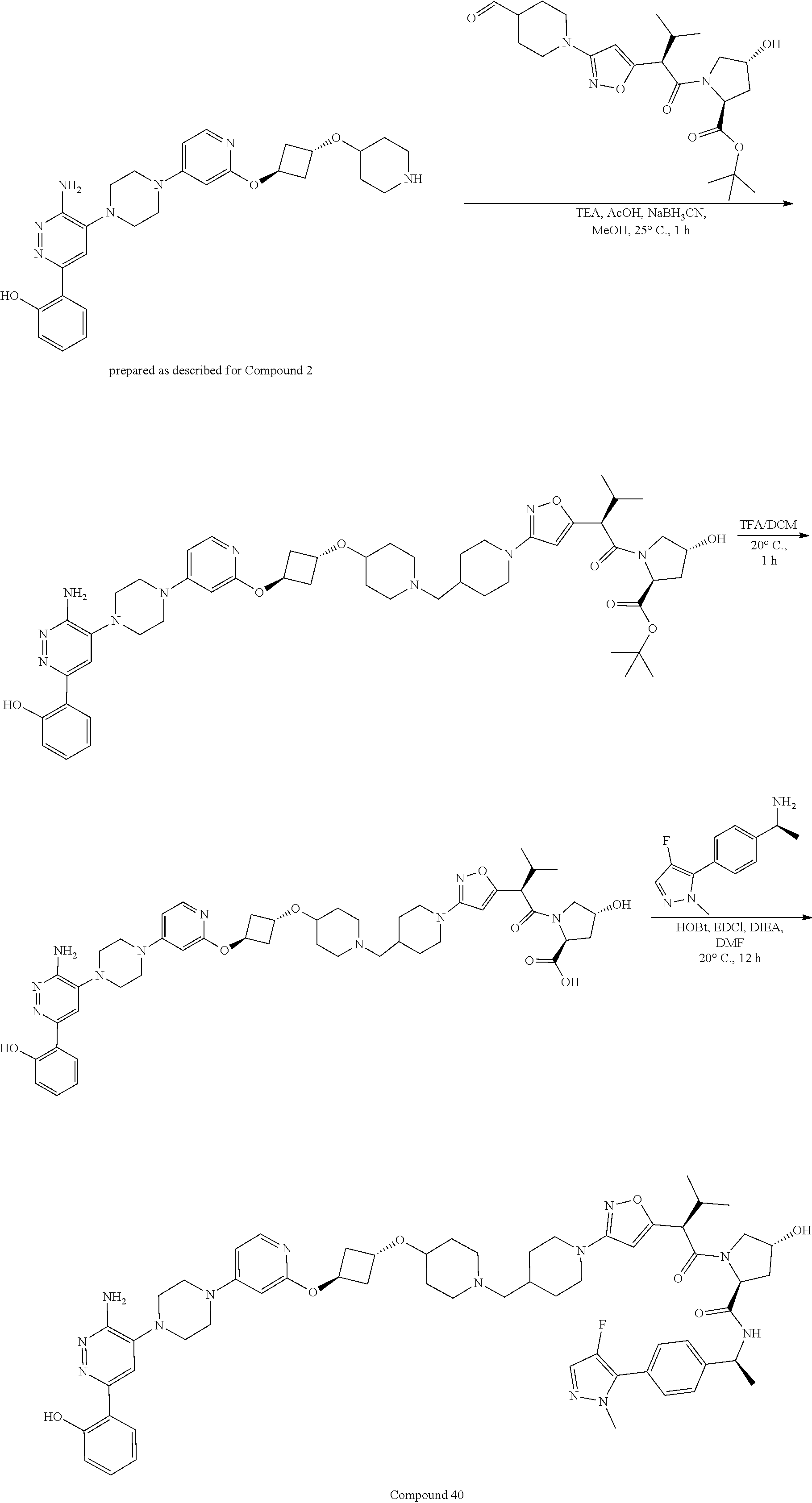

### Exemplary Synthesis of Compound 41

Prepared according to the scheme below using procedures described for other Examples above, as well as procedures commonly known to those skilled in the art.

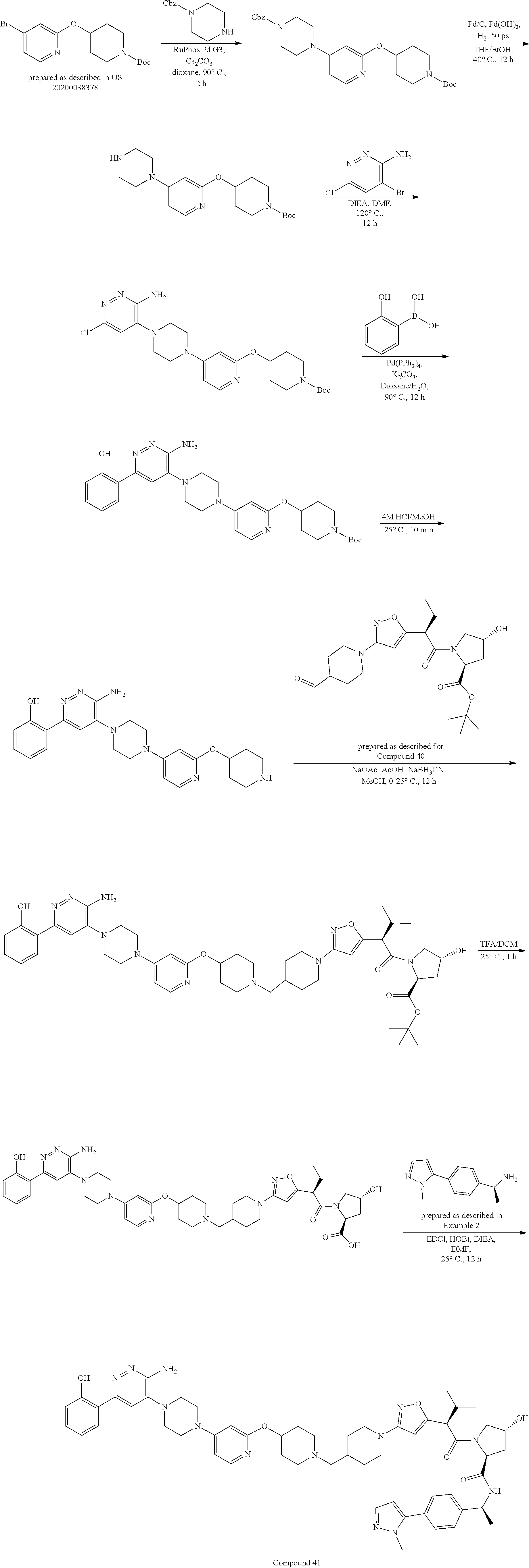

Compounds 53, 54, 146, and 147 were prepared using analogous procedures.

### Exemplary Synthesis of Compound 42

### Step 1

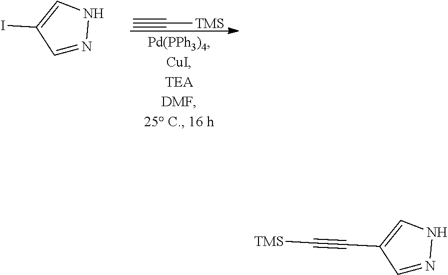

A mixture of 4-iodo-1H-pyrazole (10 g, 51.55 mmol, 1 eq), cuprous iodide (982 mg, 5.16 mmol, 0.1 eq), tetrakis[triphenylphosphine]palladium(0) (2.98 g, 2.58 mmol, 0.05 eq) and triethylamine (15.65 g, 154.66 mmol, 21.5 mL, 3 eq) in N,N-dimethylfonnamide (100 mL) was degassed with nitrogen three times. Then ethynyl(trimethyl)silane (10.13 g, 103.11 mmol, 14.3 mL, 2 eq) was added to the solution at 25° C., and the solution was degassed with nitrogen three times and stirred at 25° C. for 16 hours. The mixture was diluted with ethyl acetate (100 mL), filtered through a pad of silica gel (100-200 mesh) and washed with ethyl acetate (300 mL). The resulting solution was washed with saturated ammonium chloride aqueous solution (200 mL×2) and brine (200 mL×3). The organic layer was dried over anhydrous sodium sulfate, filtered and concentrated under vacuum to get the residue. The residue was purified by silica gel column chromatography (petroleum ether/ethyl acetate=1/0 to 6/1) to obtain the crude product. The crude product was purified by prep-HPLC (column: Waters Xbridge BEH C18 250*50 mm*10 um; mobile phase: [water (0.05% ammonia hydroxide v/v)-ACN]; B %: 35%-60%, 20 min). Trimethyl-[2-(1H-pyrazol-4-yl)ethynyl]silane (1.66 g, 10.10 mmol) was obtained as a light yellow solid.

### Step 2

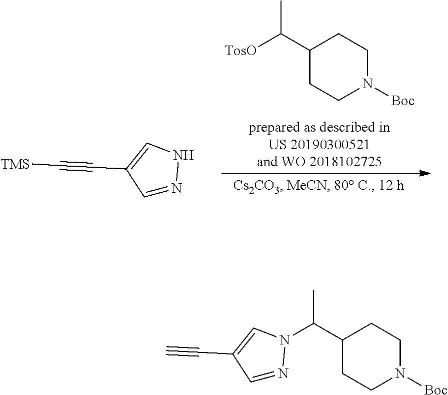

To a solution of trimethyl-[2-(1H-pyrazol-4-yl)ethynyl]silane (1.66 g, 10.10 mmol, 1 eq) and tert-butyl 4-[1-(ptolylsulfonyloxy)ethyl]piperidine-1-carboxylate (3.88 g, 10.10 mmol, 1 eq) in acetonitrile (32 mL) was added cesium carbonate (6.58 g, 20.21 mmol, 2 eq). The reaction mixture was stirred at 80° C. for 12 hours. The mixture was cooled to 25° C. and diluted with water (60 mL). The resulting solution was extracted with ethyl acetate (60 mL×2). The combined organic layer was dried over anhydrous sodium sulfate, filtered and concentrated under vacuum to get the residue. The residue was purified by prep-HPLC (column: Phenomenex luna C18 250*50 mm*10 um; mobile phase: [water (0.1% TFA)-ACN]; B %: 40%-70%, 20 min) to get tert-butyl 4-[1-(4-ethynylpyrazol-1-yl)ethyl]piperidine-1-carboxylate (2.1 g, 6.92 mmol) as a light yellow solid.

tert-Butyl 4-[1-(4-ethynylpyrazol-1-yl)ethyl]piperidine-1-carboxylate was converted to the title compound according to the scheme below using procedures described above, as well as procedures commonly known to those skilled in the art.

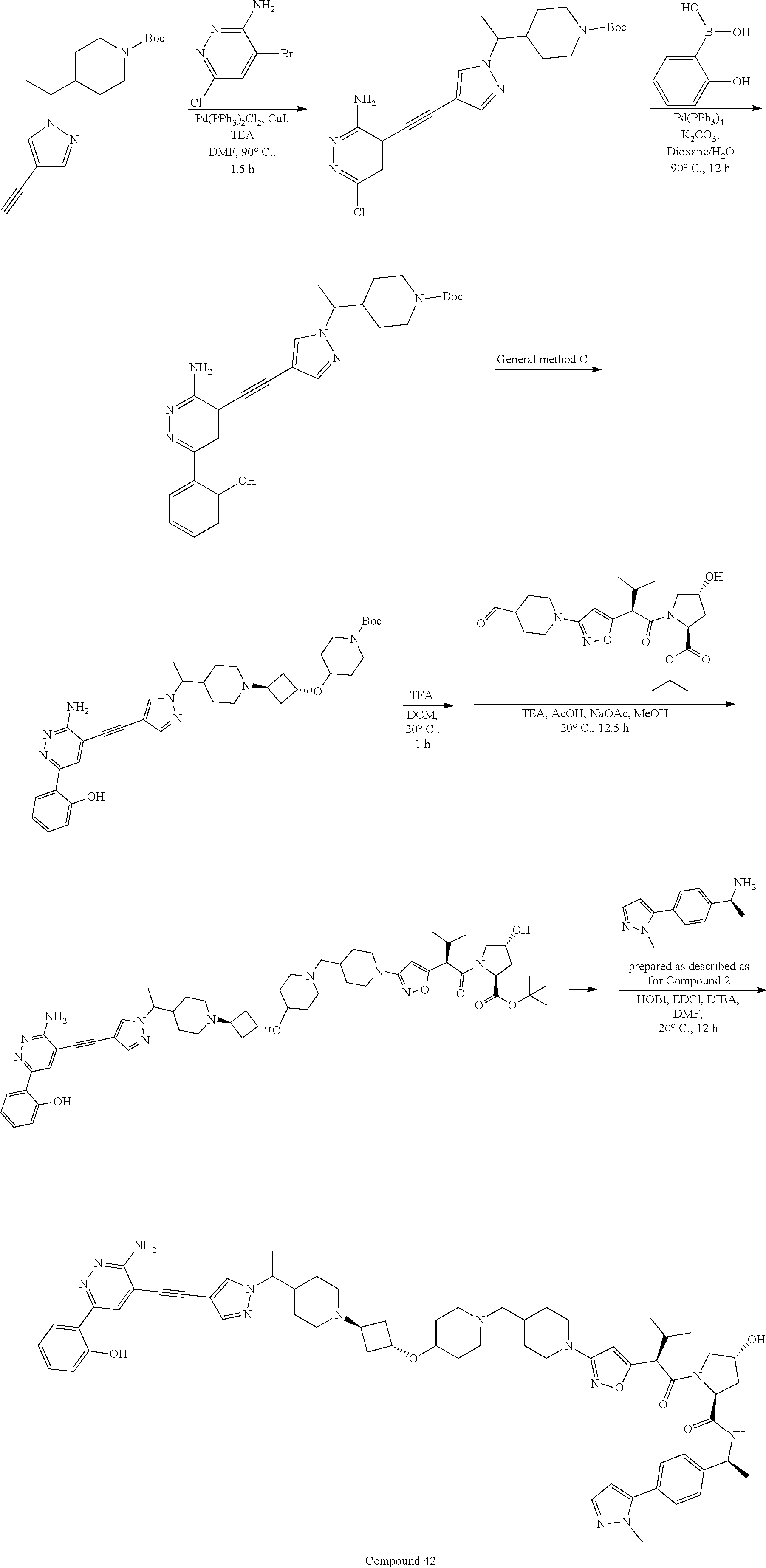

Using analogous procedures the following Examples were prepared: Compounds 78 (including last-step procedure described in Compound 14), 90, and 91.

### Exemplary Synthesis of Compound 43

Prepared according to the scheme below using procedures from Examples described above as well as those commonly known to skilled in the art.

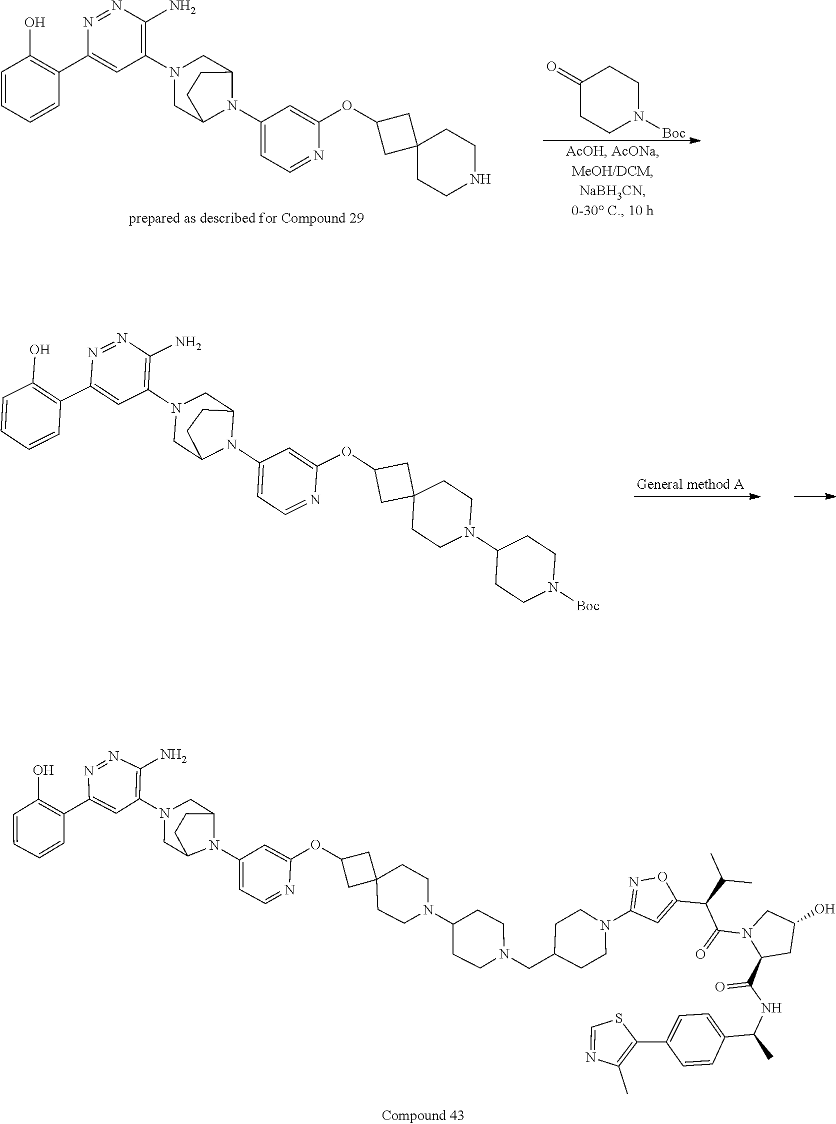 [SMILES: Nc1nnc(-c2ccccc2O)cc1N1CC2CCC(C1)N2c1ccnc(OC2CC3(CCNCC3)C2)c1 ; CC(C)(C)OC(=O)N1CCC(=O)CC1 ; CC(C)(C)OC(=O)N1CCC(N2CCC3(CC2)CC(Oc2cc(N4C5CCC4CN(c4cc(-c6ccccc6O)nnc4N)C5)ccn2)C3)CC1 ; Cc1ncsc1-c1ccc([C@H](C)NC(=O)[C@@H]2C[C@@H](O)CN2C(=O)[C@@H](c2cc(N3CCC(CN4CCC(N5CCC6(CC5)CC(Oc5cc(N7C8CCC7CN(c7cc(-c9ccccc9O)nnc7N)C8)ccn5)C6)CC4)CC3)no2)C(C)C)cc1]

### Exemplary Synthesis of Compound 44

Prepared according to the scheme below using procedures from Examples described above.

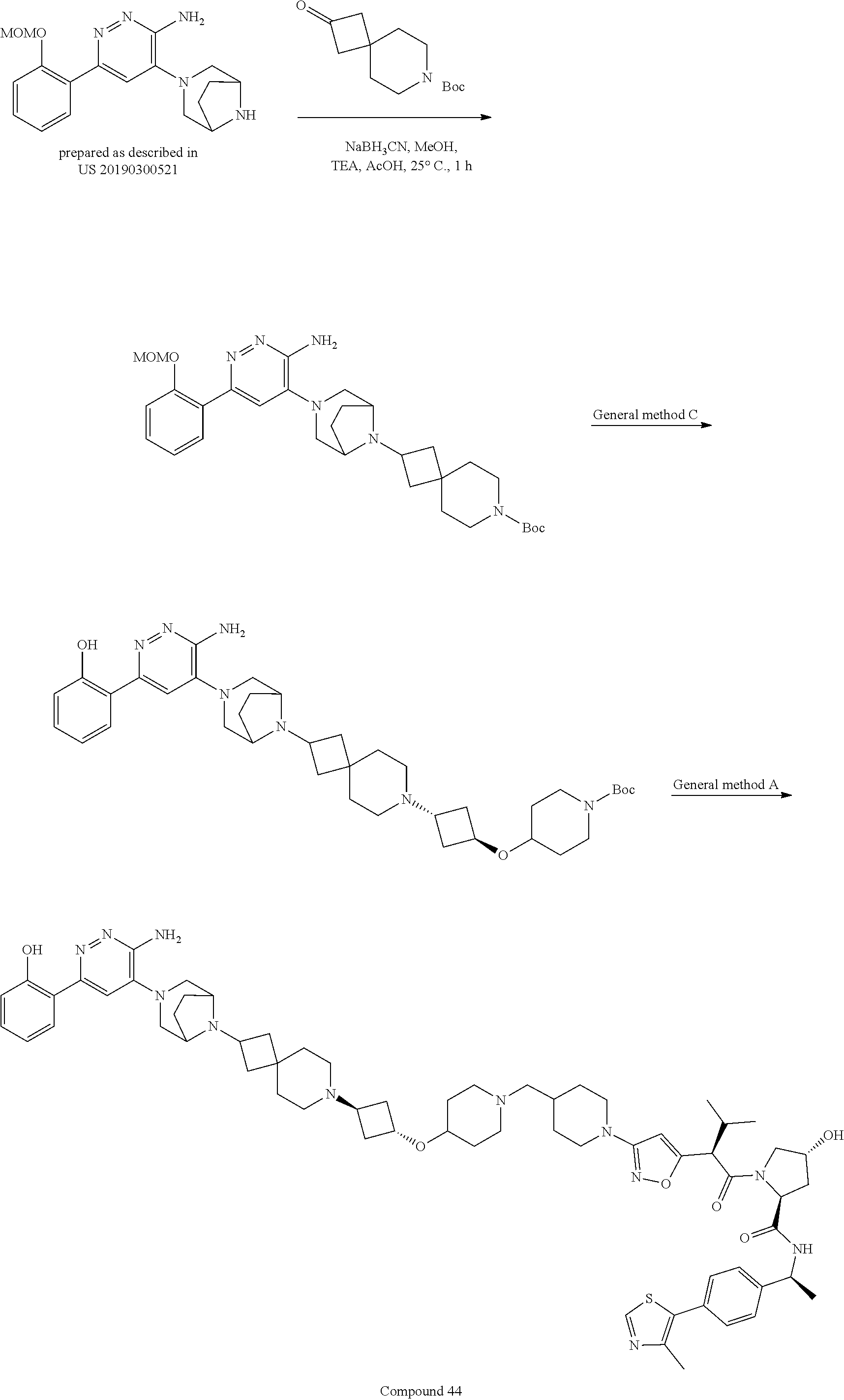

### Exemplary Synthesis of Compounds 45 and 81

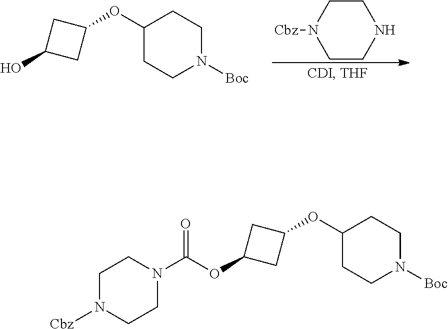 [SMILES: CC(C)(C)OC(=O)N1CCC(O[C@H]2C[C@H](O)C2)CC1 ; O=C(OCc1ccccc1)N1CCNCC1 ; CC(C)(C)OC(=O)N1CCC(O[C@H]2C[C@H](OC(=O)N3CCN(C(=O)OCc4ccccc4)CC3)C2)CC1]

A solution of tert-butyl 4-(3-hydroxycyclobutoxy)piperidine-1-carboxylate (610 mg, 2.25 mmol, 1.0 eq) and CDI (383 mg, 2.36 mmol, 1.05 eq) in anhydrous THF (12 mL) was stirred at 20° C. for 2 hours under N₂. Then benzyl piperazine-1-carboxylate (495 mg, 2.25 mmol, 1.0 eq) was added, followed by TEA (455 mg, 4.50 mmol, 2.0 eq). The mixture was stirred at 75° C. for 16 hours under N₂. Water (20 mL) was added to the mixture, which was extracted with ethyl acetate (30 mL×3). The combined extracts were washed with brine (20 mL), dried over anhydrous Na₂SO₄, filtered and concentrated under reduced pressure. The crude product was purified by Biotage® combi flash (Column: 12 g Biotage® Silica Flash column; Eluent: gradient 0-26% ethyl acetate in petroleum ether) to afford O1-benzyl O4-[3-[(1-tert-butoxycarbonyl-4-piperidyl)oxy]cyclobutyl]piperazine-1,4-dicarboxylate (530 mg, 0.95 mmol) as a colorless gum.

O1-benzyl O4-[3-[(1-tert-butoxycarbonyl-4-piperidyl)oxy]cyclobutyl]piperazine-1,4-dicarboxylate was converted to the title compound according to the scheme below using procedures described above, as well as general procedures known to those skilled in the art.

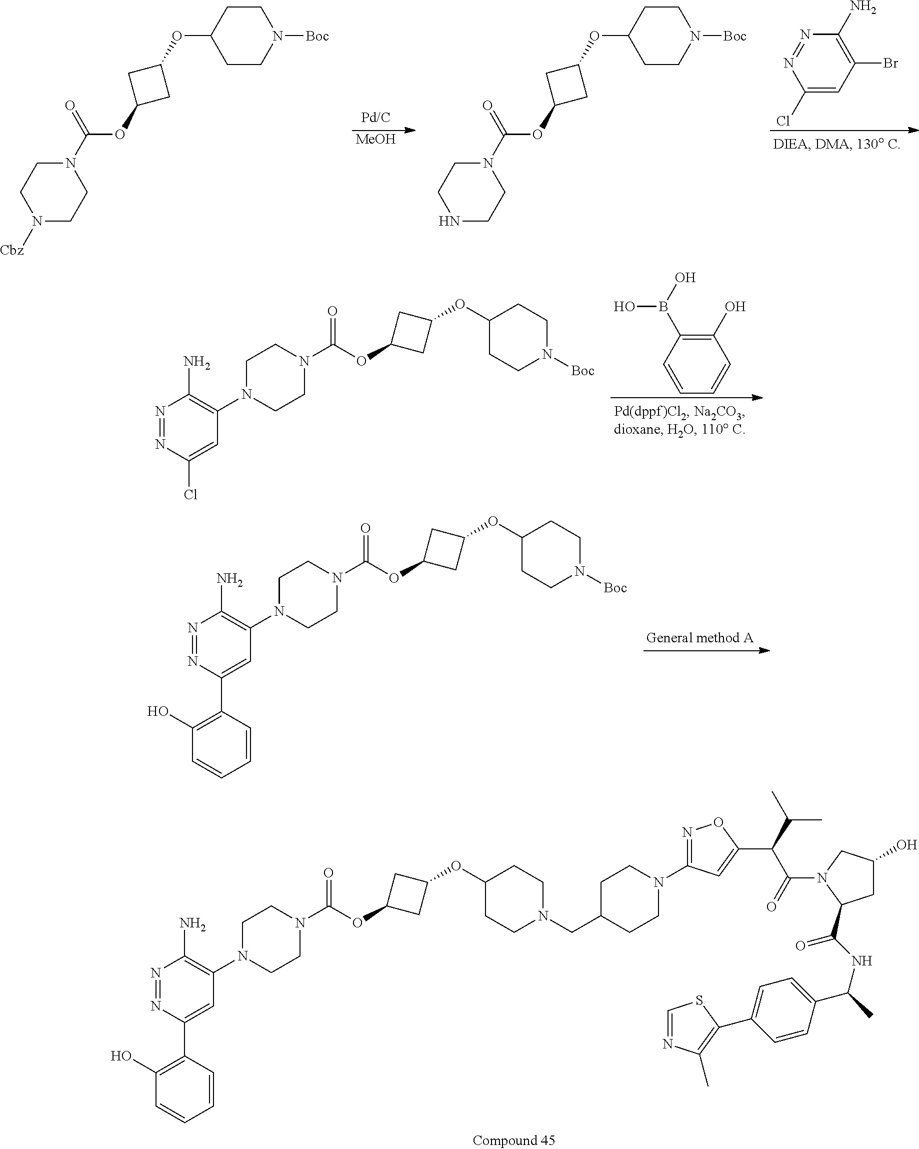

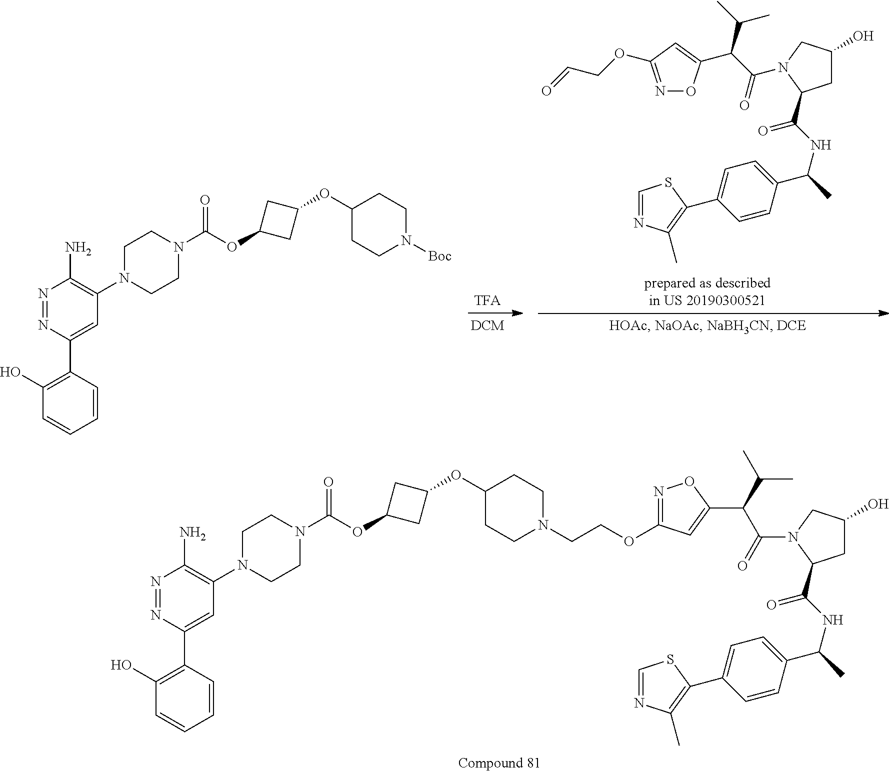 [SMILES: CC(C)(C)OC(=O)N1CCC(O[C@H]2C[C@H](OC(=O)N3CCN(c4cc(-c5ccccc5O)nnc4N)CC3)C2)CC1 ; Cc1ncsc1-c1ccc([C@H](C)NC(=O)[C@@H]2C[C@@H](O)CN2C(=O)[C@@H](c2cc(OCC=O)no2)C(C)C)cc1 ; Cc1ncsc1-c1ccc([C@H](C)NC(=O)[C@@H]2C[C@@H](O)CN2C(=O)[C@@H](c2cc(OCCN3CCC(O[C@H]4C[C@H](OC(=O)N5CCN(c6cc(-c7ccccc7O)nnc6N)CC5)C4)CC3)no2)C(C)C)cc1]

Using analogous procedures and those described above for Compound 2, Compounds 114 and 115 were prepared.

### Exemplary Synthesis of Compound 47

Prepared according to the scheme below using procedures described above, as well as procedures commonly known to those skilled in the art.

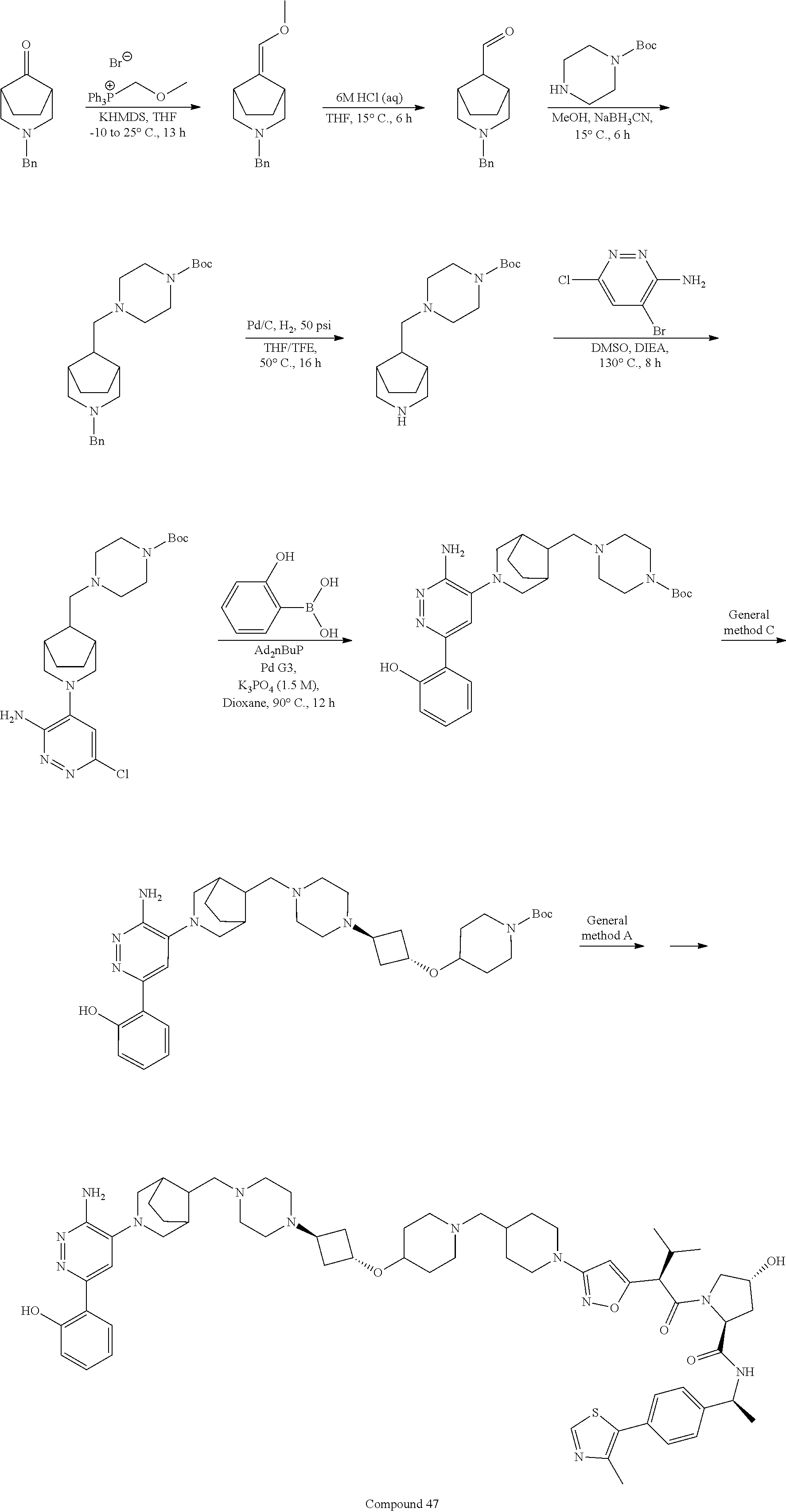

### Exemplary Synthesis of Compound 49

Prepared according to the schemes below using procedures described for other Examples above, as well as general procedures commonly known to those skilled in the art.

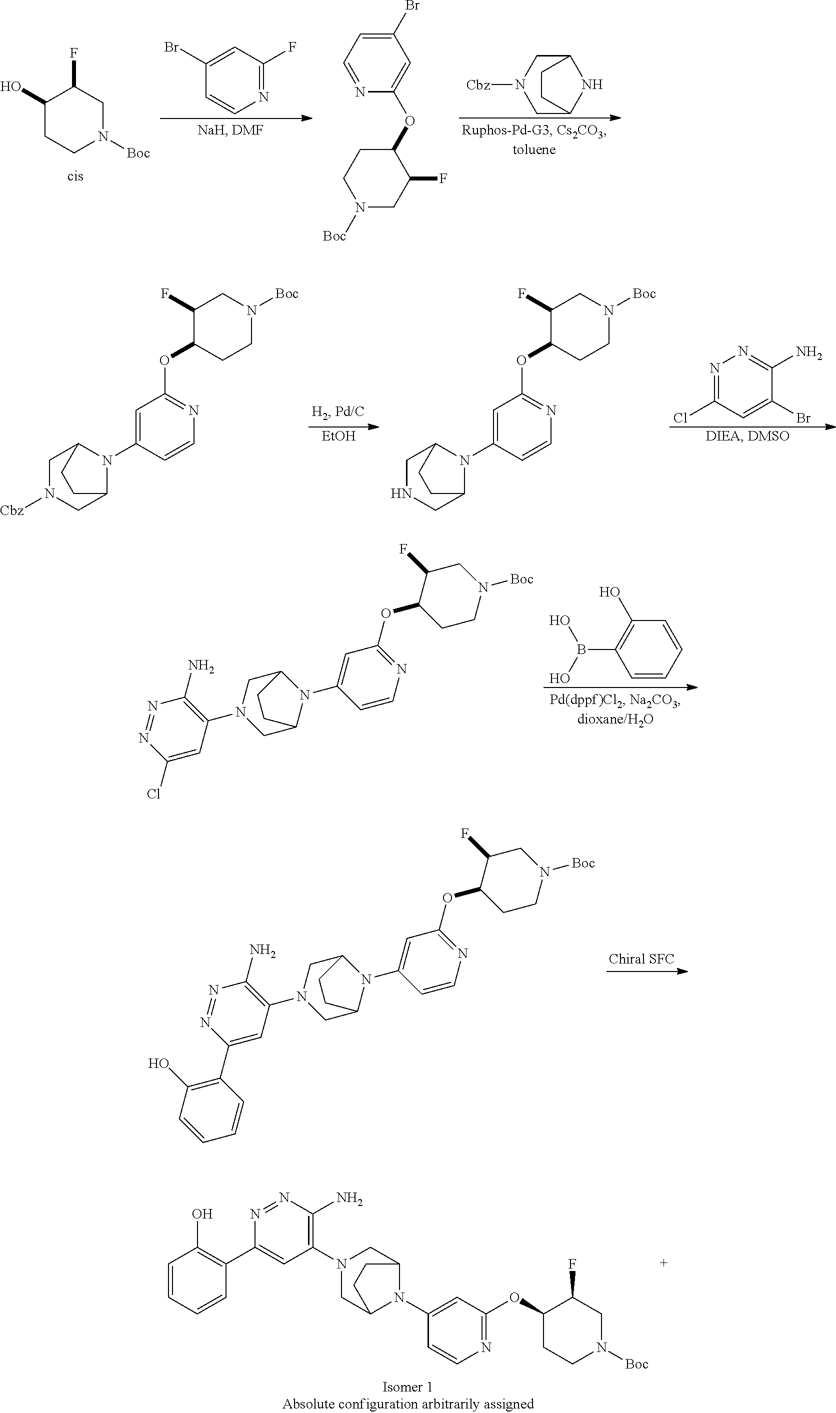

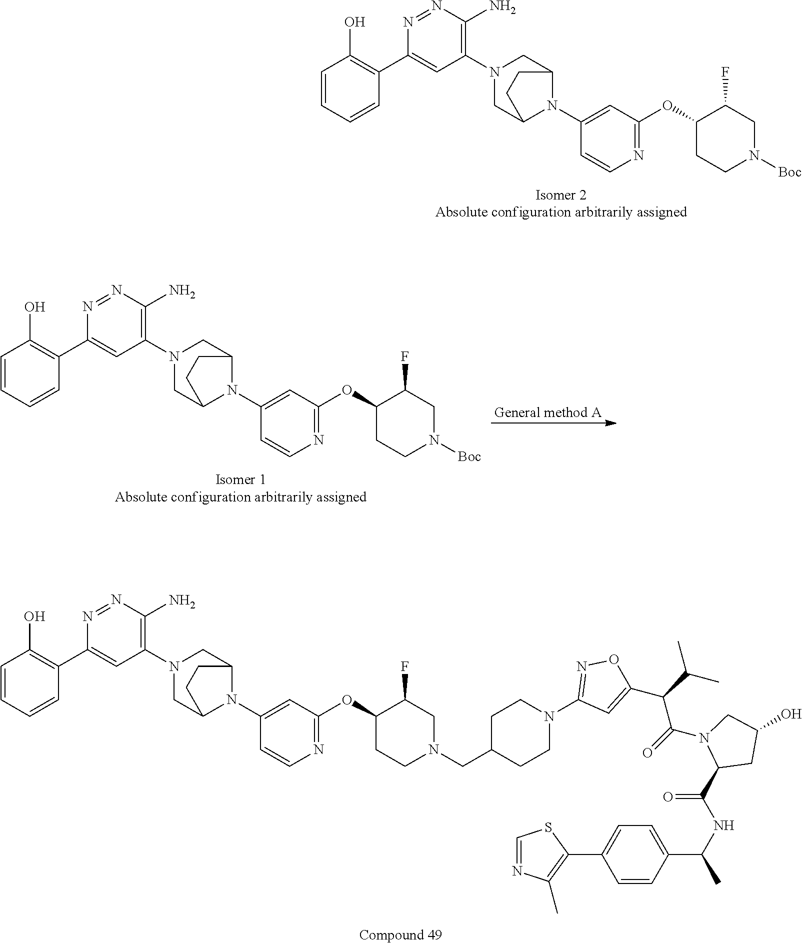 [SMILES: CC(C)(C)OC(=O)N1CC[C@H](Oc2cc(N3C4CCC3CN(c3cc(-c5ccccc5O)nnc3N)C4)ccn2)[C@H](F)C1 ; CC(C)(C)OC(=O)N1CC[C@@H](Oc2cc(N3C4CCC3CN(c3cc(-c5ccccc5O)nnc3N)C4)ccn2)[C@@H](F)C1 ; Cc1ncsc1-c1ccc([C@H](C)NC(=O)[C@@H]2C[C@@H](O)CN2C(=O)[C@@H](c2cc(N3CCC(CN4CC[C@@H](Oc5cc(N6C7CCC6CN(c6cc(-c8ccccc8O)nnc6N)C7)ccn5)[C@@H](F)C4)CC3)no2)C(C)C)cc1]

Compound 50 was prepared using an analogous procedures.

Using analogous procedures and tert-butyl trans-3-fluoro-4-hydroxypiperidine-1-carboxylate as the starting material the Compounds 59 and 60 were prepared.

### Exemplary Synthesis of Compound 56

### Step 1

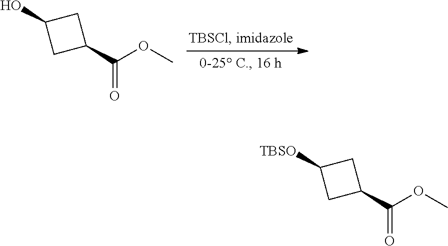

To a mixture of methyl 3-hydroxycyclobutanecarboxylate (2.0 g, 15.37 mmol, 1.0 eq) and 1H-imidazole (3.14 g, 46.10 mmol, 3.0 eq) in dichloromethane (30 mL) was added tert-butyldimethylsilyl chloride (3.47 g, 23.05 mmol, 1.5 eq) at 15° C. under nitrogen. The mixture was stirred at 15° C. for 16 hours. The mixture was washed with brine (30 mL×3), dried with anhydrous sodium sulphate, filtered and concentrated under vacuum. The residue was purified by silica gel chromatography (petroleum ether/ethyl acetate=100/1, 50/1) to afford methyl 3-[tert-butyl(dimethyl)silyl] oxycyclobutanecarboxylate (3.2 g, 13.09 mmol) as a colorless oil.

### Step 2

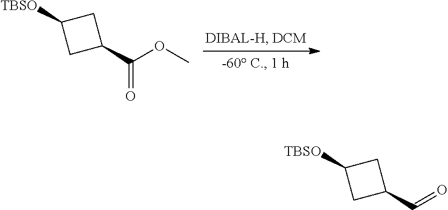 [SMILES: COC(=O)[C@H]1C[C@@H](O[Si](C)(C)C(C)(C)C)C1 ; CC(C)(C)[Si](C)(C)O[C@H]1C[C@@H](C=O)C1]

To a mixture of methyl 3-[tert-butyl(dimethyl)silyl]oxycyclobutanecarboxylate (3.2 g, 13.09 mmol, 1 eq) in dichloromethane (120 mL) was added diisobutyl aluminium hydride (1 M, 17.0 mL, 1.3 eq) at −60° C. under nitrogen. The mixture was stirred at −60° C. for 1 hours. The reaction mixture was quenched by the addition methanol (3 mL) at −70° C., and then diluted with dichloromethane (100 mL) and saturated sodium potassium tartrate solution (200 mL). The mixture was stirred stirred for 6 hr, then extracted with dichloromethane (100 mL). The combined organic layers were dried over anhydrous sodium sulphate, filtered and concentrated under reduced pressure to give a residue. The residue was purified by silica gel chromatography (petroleum ether/ethyl acetate=100/1 to 10/1) to afford 3-[tert-butyl(dimethyl) silyl]oxycyclobutanecarbaldehyde (2.5 g, 11.66 mmol) as a colorless oil.

### Step 3

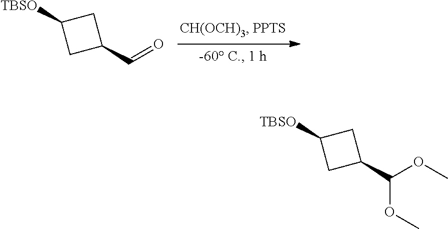 [SMILES: CC(C)(C)[Si](C)(C)O[C@H]1C[C@@H](C=O)C1 ; COC(OC)[C@H]1C[C@@H](O[Si](C)(C)C(C)(C)C)C1]

To a mixture of 3-[tert-butyl(dimethyl)silyl]oxycyclobutanecarbaldehyde (2.50 g, 11.66 mmol, 1.0 eq) and trimethoxymethane (15.09 g, 142.23 mmol, 12.2 eq) in methanol (6 mL) was added pyridinium p-toluenesulfonate (293 mg, 1.17 mmol, 0.1 eq) in one portion at 15° C. under nitrogen. The mixture was stirred at 15° C. for 16 hours. The mixture was poured into saturated sodium bicarbonate solution (10 mL) and stirred for 15 minutes. The aqueous phase was extracted with ethyl acetate (30 mL×2). The combined organic phase was washed with brine (30 mL×2), dried with anhydrous sodium sulphate, filtered and concentrated under vacuum. The residue was purified by silica gel column chromatography (petroleum ether/ethyl acetate=100/1 to 10/1) to afford tert-butyl-[3-(dimethoxymethyl)cyclobutoxy]-dimethyl-silane (2.2 g, 8.45 mmol) as a colorless oil.

### Step 4

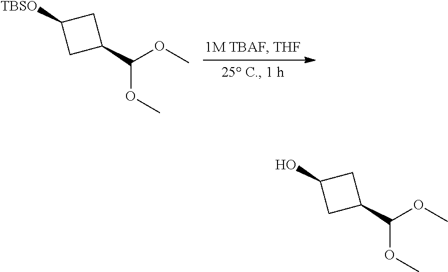

To a mixture of tert-butyl-[3-(dimethoxymethyl)cyclobutoxy]-dimethyl-silane (2.2 g, 8.45 mmol, 1.0 eq) in tetrahydrofuran (60 mL) was added tetrabutylammonium fluoride (1 M, 12.7 mL, 1.5 eq) in one portion at 15° C. under nitrogen. The mixture was stirred at 15° C. for 2 hours. The mixture was concentrated in reduced pressure at 45° C. The residue was diluted with ethyl acetate (50 mL), washed with brine (30 mL×3), dried with anhydrous sodium sulphate, filtered and concentrated under vacuum. The residue was purified by silica gel column chromatography (petroleum ether/ethyl acetate=10/1 to 1/1) to afford 3-(dimethoxymethyl)cyclobutanol (1.01 g, 6.91 mmol) as a yellow oil.

### Step 5

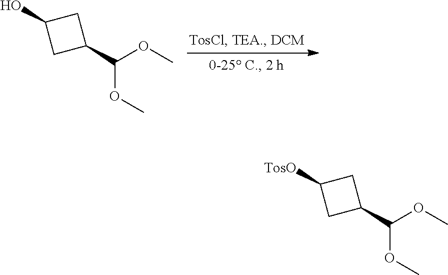

To a mixture of 3-(dimethoxymethyl)cyclobutanol (1.01 g, 6.91 mmol, 1.0 eq), 4-dimethylaminopyridine (84 mg, 0.69 mmol, 0.1 eq) and p-toluenesulfonyl chloride (2.63 g, 13.82 mmol, 2.0 eq) in dichloromethane (20 mL) was added triethylamine (2.1 g, 20.73 mmol, 2.9 mL, 3.0 eq) in one portion at 15° C. under nitrogen. The mixture was stirred at 15° C. for 6 hours. The mixture was concentrated under reduced pressure at 45° C. The residue was purified by silica gel column chromatography (petroleum ether/ethyl acetate=20/1, 10/1) to afford [3-(dimethoxymethyl)cyclobutyl] 4-methylbenzenesulfonate (1.3 g, 4.33 mmol) as a yellow oil.

[3-(dimethoxymethyl)cyclobutyl] 4-methylbenzenesulfonate was converted to the title compound according to the scheme below using procedures commonly known to those skilled in the art.

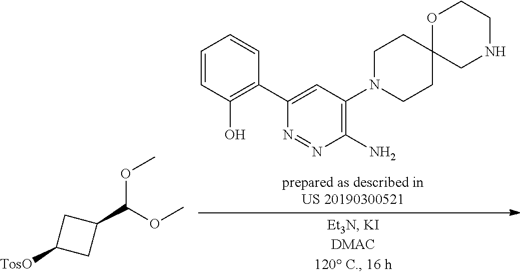

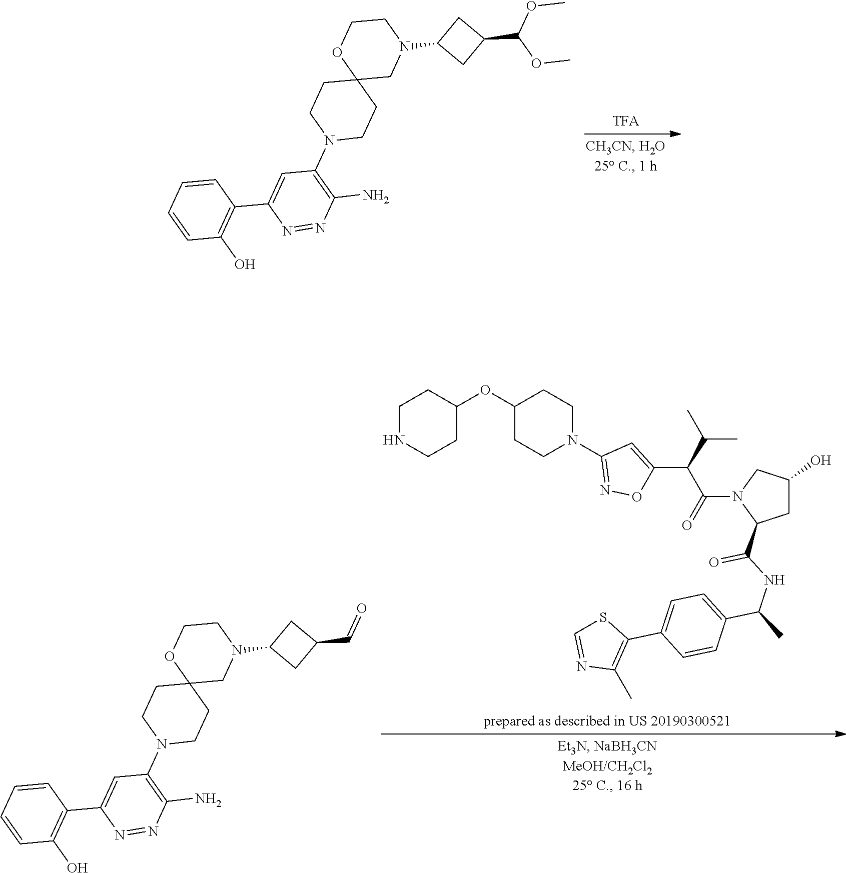 [SMILES: COC(OC)[C@H]1C[C@H](N2CCOC3(CCN(c4cc(-c5ccccc5O)nnc4N)CC3)C2)C1 ; Nc1nnc(-c2ccccc2O)cc1N1CCC2(CC1)CN([C@H]1C[C@H](C=O)C1)CCO2 ; Cc1ncsc1-c1ccc([C@H](C)NC(=O)[C@@H]2C[C@@H](O)CN2C(=O)[C@@H](c2cc(N3CCC(OC4CCNCC4)CC3)no2)C(C)C)cc1]

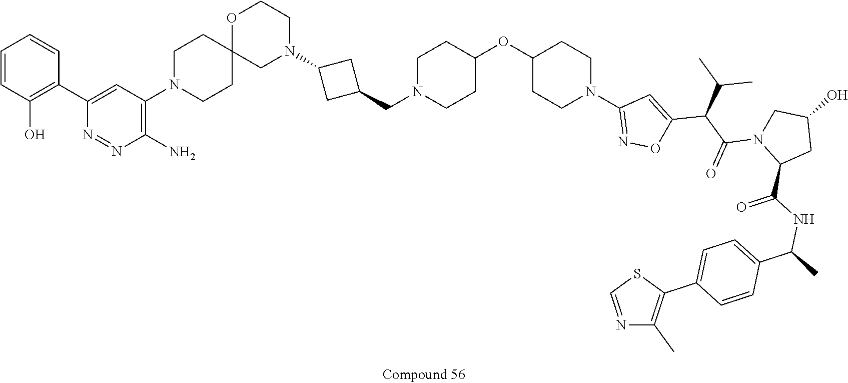 [SMILES: Cc1ncsc1-c1ccc([C@H](C)NC(=O)[C@@H]2C[C@@H](O)CN2C(=O)[C@@H](c2cc(N3CCC(OC4CCN(C[C@H]5C[C@H](N6CCOC7(CCN(c8cc(-c9ccccc9O)nnc8N)CC7)C6)C5)CC4)CC3)no2)C(C)C)cc1]

### Exemplary Synthesis of Compound 57

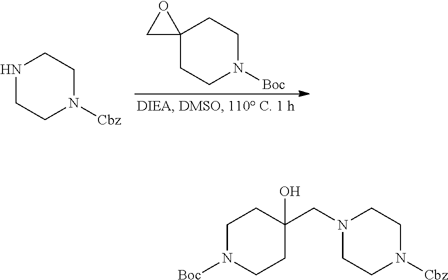 [SMILES: O=C(OCc1ccccc1)N1CCNCC1 ; CC(C)(C)OC(=O)N1CCC2(CC1)CO2 ; CC(C)(C)OC(=O)N1CCC(O)(CN2CCN(C(=O)OCc3ccccc3)CC2)CC1]

To a solution of benzyl piperazine-1-carboxylate (1 g, 4.54 mmol, 0.9 mL, 1 eq) and tert-butyl 1-oxa-6-azaspiro[2.5]octane-6-carboxylate (968 mg, 4.54 mmol, 1 eq) in dimethylsulfoxide (5 mL) was added N,N-diisopropylethylamine (1.17 g, 9.08 mmol, 1.6 mL, 2 eq). The mixture was stirred at 110° C. for 1 hour. 30 mL water was added to the mixture, then the mixture was extracted with ethyl acetate (30 mL×3). The combined organic phase was washed with brine (30 mL), dried with anhydrous sodium sulfate, filtered and concentrated in vacuum. The residue was purified by silica gel chromatography (petroleum ether/ethyl acetate=30/1 to 3/1). Benzyl 4-[(1-tert-butoxycarbonyl-4-hydroxy-4-piperidyl)methyl]piperazine-1-carboxylate (1.2 g, 2.77 mmol) was obtained as a yellow oil.

Benzyl 4-[(1-tert-butoxycarbonyl-4-hydroxy-4-piperidyl)methyl]piperazine-1-carboxylate was converted to the title compound according to the scheme below using procedures described for other Examples above.

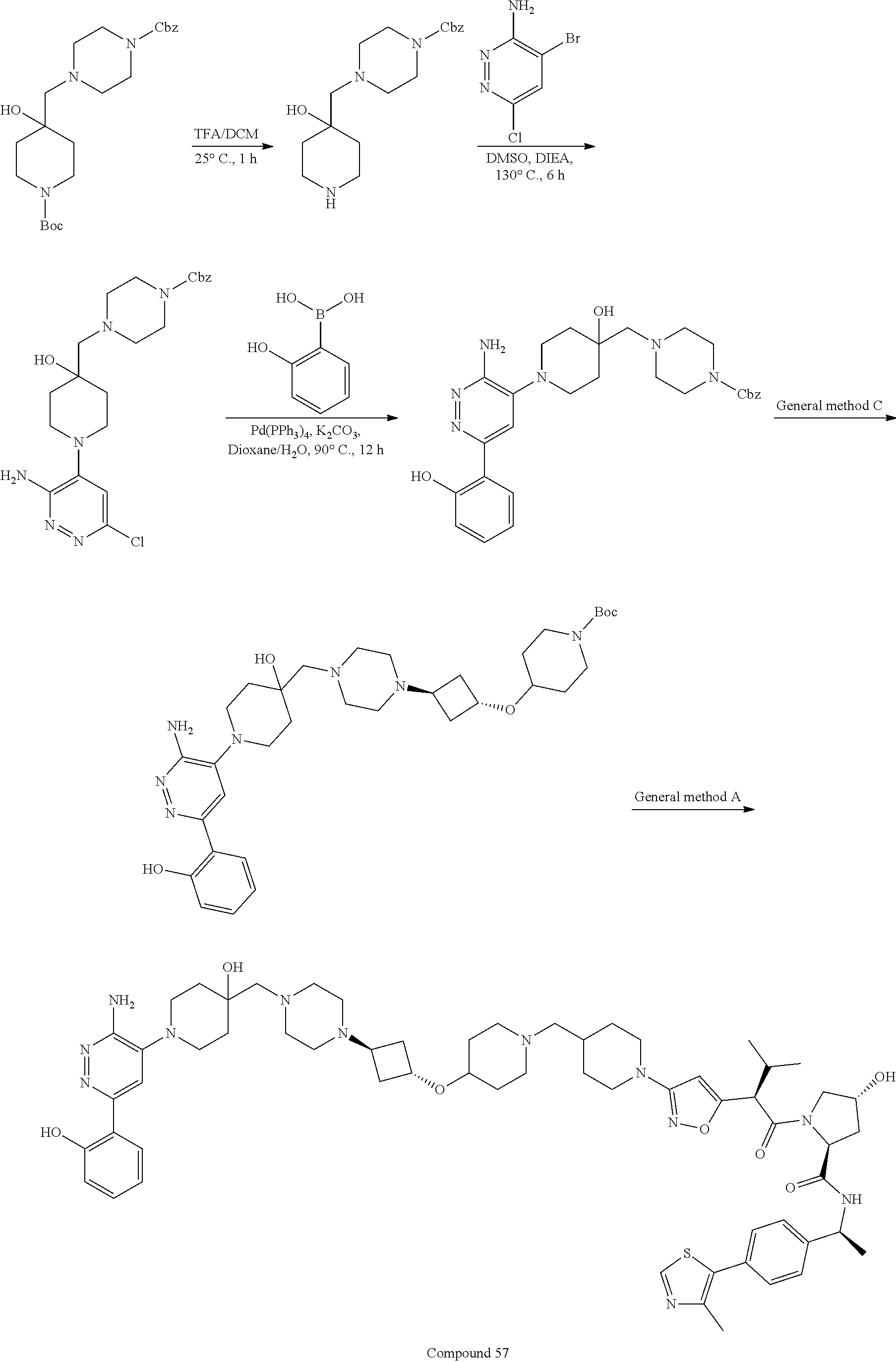

### Exemplary Synthesis of Compound 58

Prepared according to the scheme below using procedures described for other Examples above as well as those commonly known to skilled in the art.

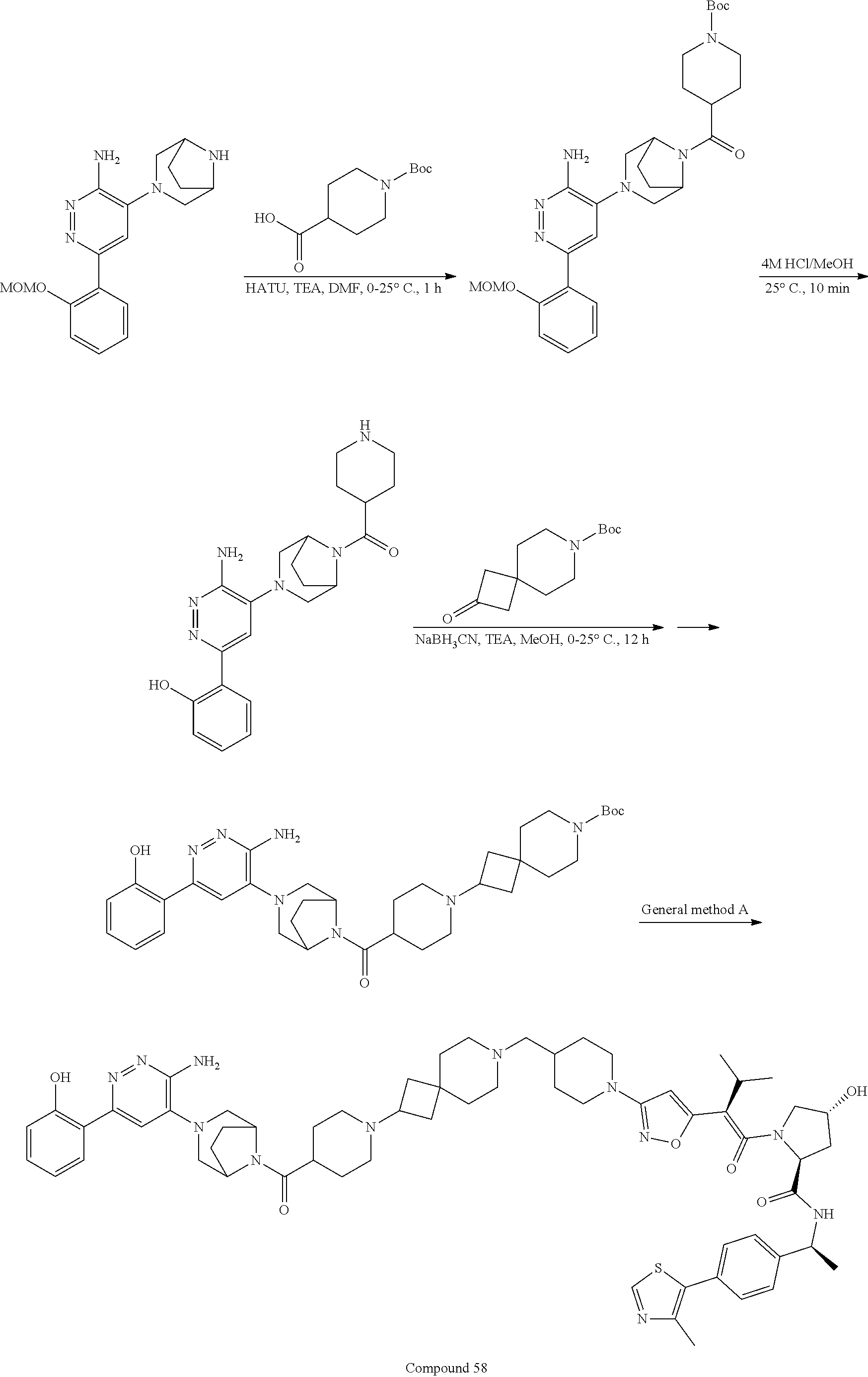

### Exemplary Synthesis of Compound 61

Prepared according to the schemes below using procedures described for other Examples above, as well as general procedures known to those skilled in the art.

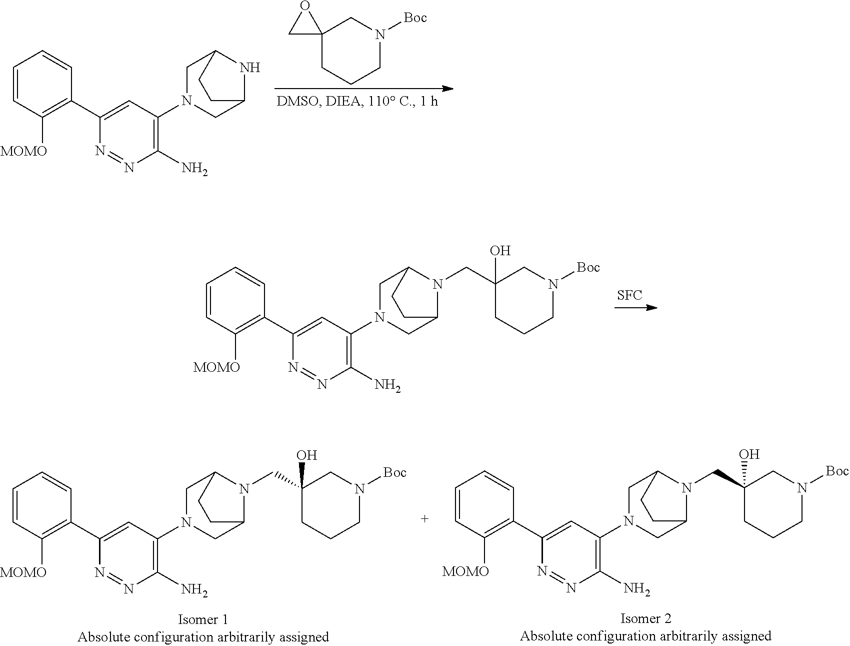

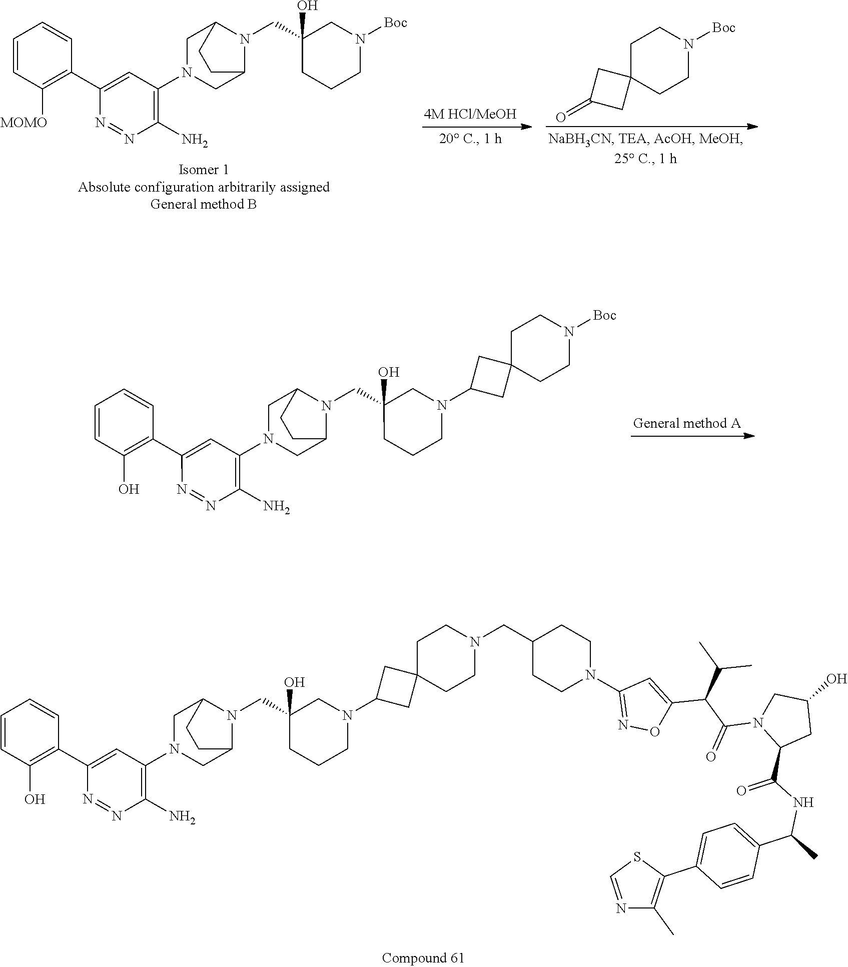

Compound 71 was prepared using analogous procedures.

### Exemplary Synthesis of Compound 62

Prepared according to the schemes below using procedures described for other Examples above, as well as general procedures known to those skilled in the art.

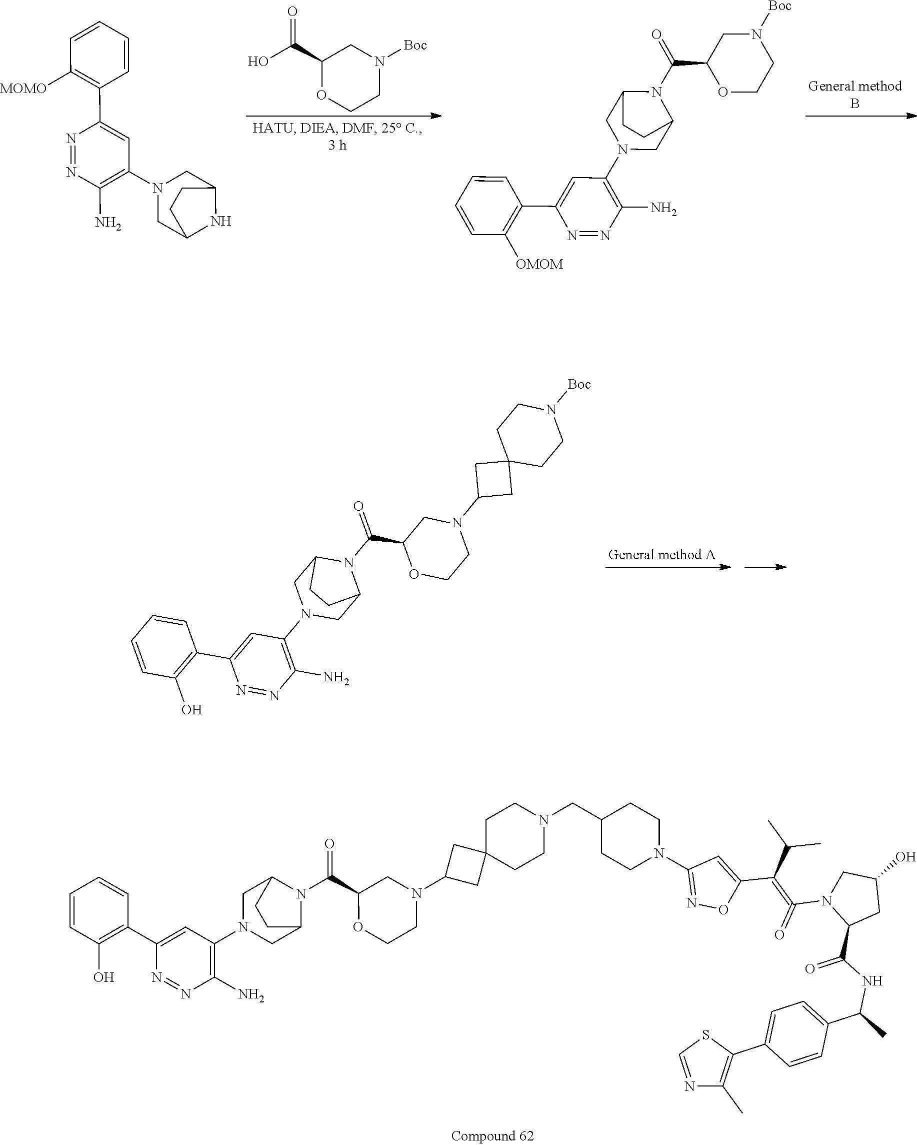

### Exemplary Synthesis of Compound 63

Prepared according to the schemes below using procedures described for other Examples above, as well as general procedures known to those skilled in the art.

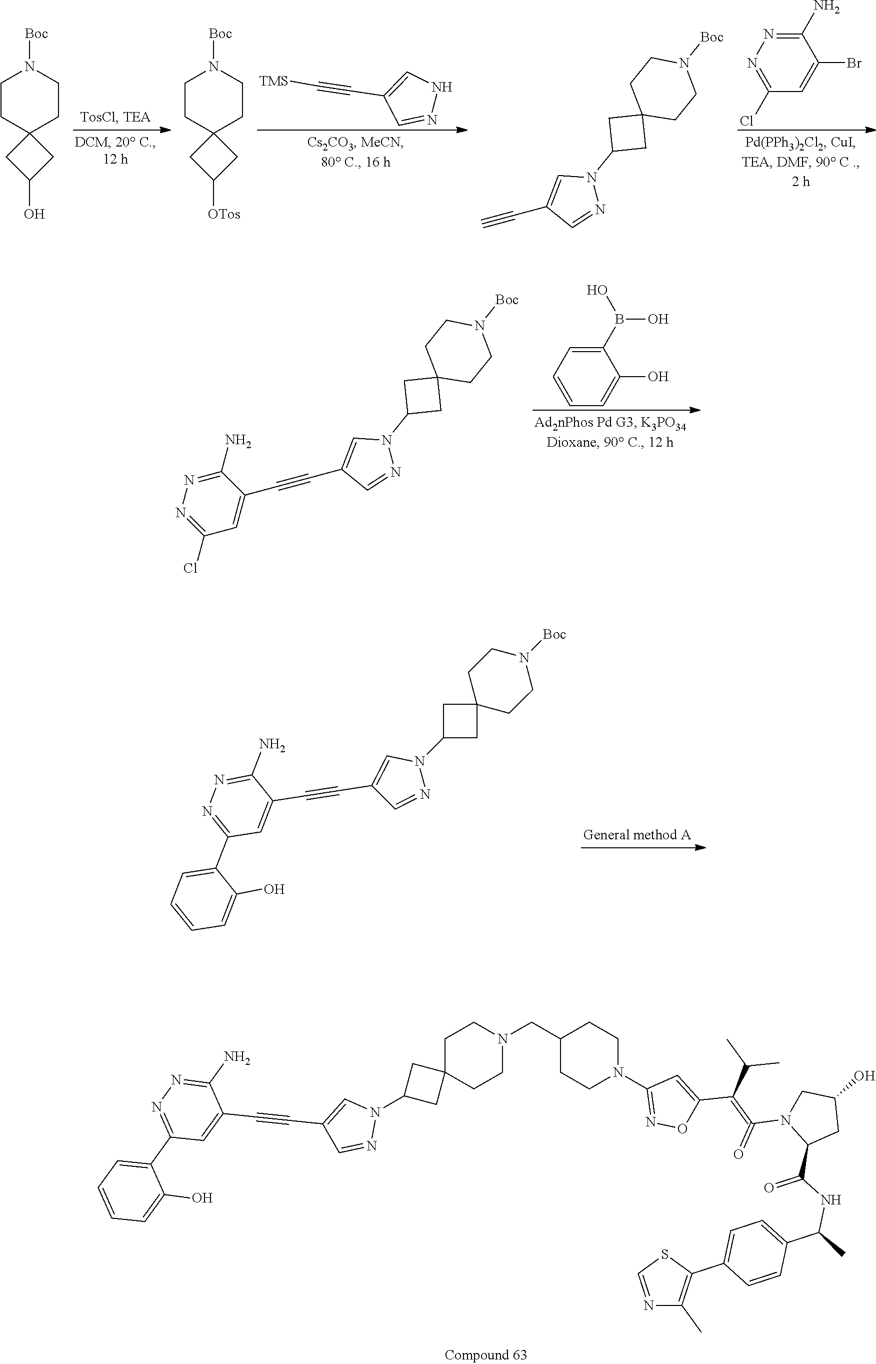

### Exemplary Synthesis of Compounds 64 and 65

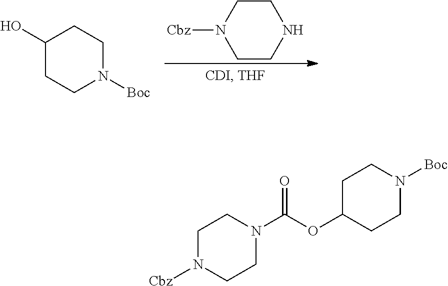 [SMILES: CC(C)(C)OC(=O)N1CCC(O)CC1 ; O=C(OCc1ccccc1)N1CCNCC1 ; CC(C)(C)OC(=O)N1CCC(OC(=O)N2CCN(C(=O)OCc3ccccc3)CC2)CC1]

To a solution of tert-butyl 4-hydroxypiperidine-1-carboxylate (4.0 g, 19.87 mmol, 1.0 eq) in tetrahydrofuran (100 mL) was added CDI (3.2 g, 19.87 mmol, 1.0 eq), and the mixture was stirred at 20° C. for 2 hours under N₂. Then benzyl piperazine-1-carboxylate (4.4 g, 19.87 mmol, 1.0 eq) was added, followed by TEA (4.02 g, 39.75 mmol, 2.0 eq). The mixture was stirred at 75° C. for 16 hours under N₂. The reaction mixture was quenched by water (100 mL) and extracted with ethyl acetate (100 mL×3). The combined organic layer was washed with brine (200 mL), dried over Na₂SO₄, filtered and concentrated. The crude product was purified by Biotage® combi flash (Column: 120 g Biotage® Silica Flash column; Eluent: gradient 0-42% methyl tert-butyl ether in petroleum ether). O1-benzyl O4-(1-tert-butoxycarbonyl-4-piperidyl)piperazine-1,4-dicarboxylate (3.85 g, 7.62 mmol) was obtained as a white solid.

O1-benzyl O4-(1-tert-butoxycarbonyl-4-piperidyl)piperazine-1,4-dicarboxylate was converted to the title compounds as described in the schemes below.

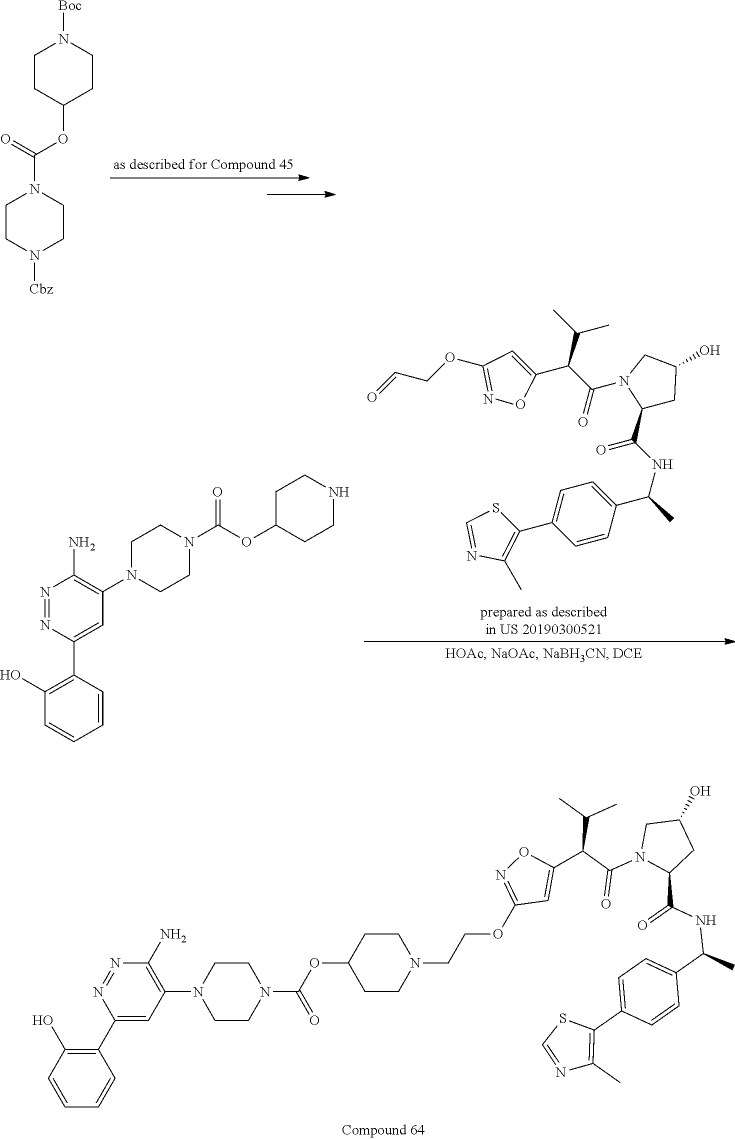 [SMILES: CC(C)(C)OC(=O)N1CCC(OC(=O)N2CCN(C(=O)OCc3ccccc3)CC2)CC1 ; Nc1nnc(-c2ccccc2O)cc1N1CCN(C(=O)OC2CCNCC2)CC1 ; Cc1ncsc1-c1ccc([C@H](C)NC(=O)[C@@H]2C[C@@H](O)CN2C(=O)[C@@H](c2cc(OCC=O)no2)C(C)C)cc1 ; Cc1ncsc1-c1ccc([C@H](C)NC(=O)[C@@H]2C[C@@H](O)CN2C(=O)[C@@H](c2cc(OCCN3CCC(OC(=O)N4CCN(c5cc(-c6ccccc6O)nnc5N)CC4)CC3)no2)C(C)C)cc1]

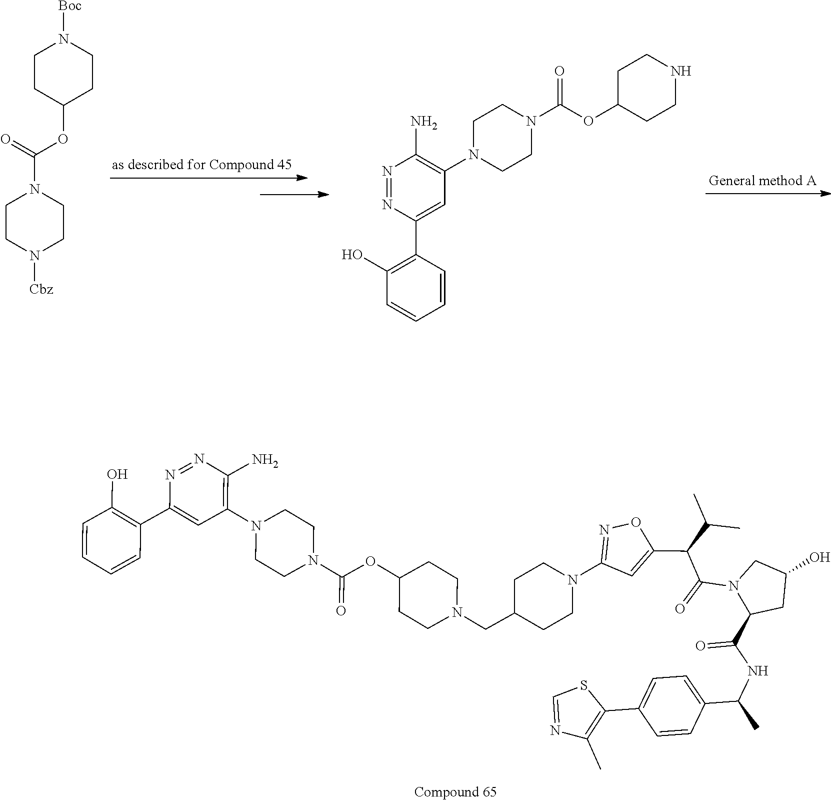 [SMILES: CC(C)(C)OC(=O)N1CCC(OC(=O)N2CCN(C(=O)OCc3ccccc3)CC2)CC1 ; Nc1nnc(-c2ccccc2O)cc1N1CCN(C(=O)OC2CCNCC2)CC1 ; Cc1ncsc1-c1ccc([C@H](C)NC(=O)[C@@H]2C[C@@H](O)CN2C(=O)[C@@H](c2cc(N3CCC(CN4CCC(OC(=O)N5CCN(c6cc(-c7ccccc7O)nnc6N)CC5)CC4)CC3)no2)C(C)C)cc1]

### Exemplary Synthesis of Compound 68

### Step 1

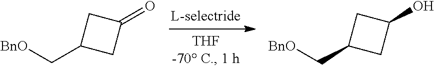 [SMILES: O=C1CC(COCc2ccccc2)C1 ; O[C@H]1C[C@@H](COCc2ccccc2)C1]

To a solution of 3-(benzyloxymethyl)cyclobutanone (3 g, 15.77 mmol, 1 eq) in tetrahydrofuran (60 mL) was added L-selectride (1 M, 18.9 mL, 1.2 eq) slowly at −70° C. The mixture was stirred at −70° C. for 1 hour. The reaction mixture was quenched by addition saturated aqueous ammonium chloride (30 mL) at 25° C., and then extracted with ethyl acetate (70 mL×3). The combined organic layers were washed with brine (30 mL), dried with anhydrous sodium sulfate, filtered and concentrated under reduced pressure to give a residue. The residue was purified by silica gel chromatography (petroleum ether/ethyl acetate=3/1). Compound 3-(benzyloxymethyl)cyclobutanol (2.8 g, 14.56 mmol) was obtained as a colorless oil.

### Step 2

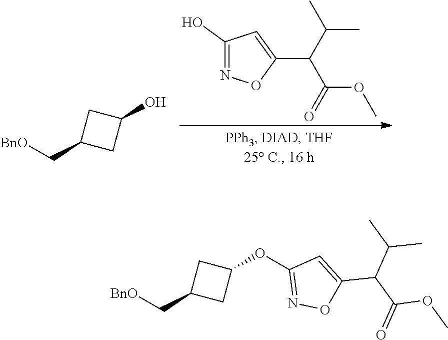 [SMILES: O[C@H]1C[C@@H](COCc2ccccc2)C1 ; COC(=O)C(c1cc(O)no1)C(C)C ; COC(=O)C(c1cc(O[C@H]2C[C@H](COCc3ccccc3)C2)no1)C(C)C]

To a solution of 3-(benzyloxymethyl)cyclobutanol (1.4 g, 7.28 mmol, 1 eq), methyl 2-(3-hydroxyisoxazol-5-yl)-3-methylbutanoate (1.74 g, 8.74 mmol, 1.2 eq) and triphenylphosphine (4.20 g, 16.02 mmol, 2.2 eq) in tetrahydrofuran (60 mL) was added diisopropylazodicarboxylate (2.94 g, 14.56 mmol, 2.8 mL, 2 eq) slowly at 25° C. The mixture was stirred at 25° C. for 16 hours. The reaction mixture was concentrated under reduced pressure to give a residue. The residue was purified by preparative HPLC (column: Phenomenex luna C18 (250*70 mm, 15 um); mobile phase: [water (0.225% FA)-ACN]; B %: 60%-90%, 30 min). Compound methyl 2-[3-[3-(benzyloxymethyl)cyclobutoxy]isoxazol-5-yl]-3-methyl-butanoate (2.3 g, 6.16 mmol) was obtained as a brown oil.

### Step 3

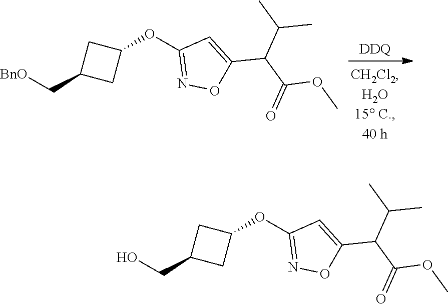

To a solution of methyl 2-[3-[3-(benzyloxymethyl)cyclobutoxy]isoxazol-5-yl]-3-methyl-butanoate (2.2 g, 5.89 mmol, 1 eq) in dichloromethane (50 mL) was added a solution of 2,3-dichloro-5,6-dicyano-1,4-benzoquinone (2.01 g, 8.84 mmol, 1.5 eq) in water (10 mL) at 15° C. The mixture was stirred at 15° C. for 40 hours. The reaction mixture was filtered and concentrated under reduced pressure to give a residue. The residue was purified by preparative HPLC (column: Phenomenex luna C18 250*50 mm*10 um); mobile phase: [water (0.225% FA)-ACN]; B %: 25%-55%, 20 min). Compound methyl 2-[3-[3-(hydroxymethyl)cyclobutoxy]isoxazol-5-yl]-3-methyl-butanoate (1.1 g, 3.88 mmol) was obtained as a brown oil.

### Step 4

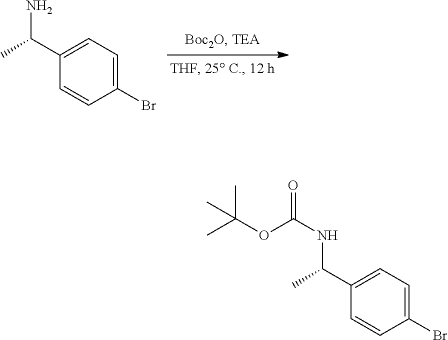

To a solution of (1S)-1-(4-bromophenyl)ethanamine (24.9 g, 124.5 mmol, 1 eq) in tetrahydrofuran (350 mL) was added triethylamine (37.8 g, 373.4 mmol, 3 eq) followed by di-tert-butyl dicarbonate (28.5 g, 130.7 mmol, 30 mL, 1.05 eq) dropwise at 0° C. under nitrogen. The mixture was then stirred at 25° C. for 12 hours. The reaction mixture was concentrated under reduced pressure to remove tetrahydrofuran. Water (400 mL) was added, and the mixture was stirred for 1 minute. The aqueous phase was extracted with ethyl acetate (200 mL×3). The combined organic phase was washed with brine (200 mL×2), dried over anhydrous sodium sulfate, filtered, and concentrated in vacuum. The crude product was triturated with petroleum ether (250 mL). Compound tert-butyl N-[(1S)-1-(4-bromophenyl)ethyl]carbamate (34.5 g, 114.93 mmol, 92% yield) was obtained as a white solid.

### Step 5

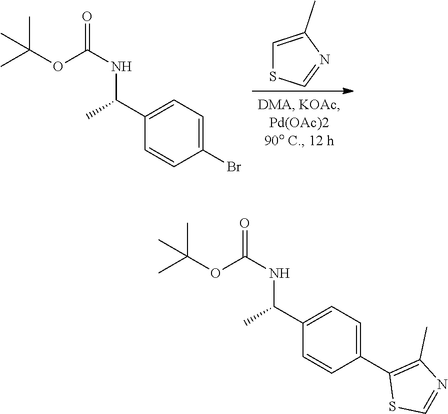

To a solution of tert-butyl N-[(1S)-1-(4-bromophenyl)ethyl]carbamate (14.5 g, 48.30 mmol, 1 eq) and 4-methylthiazole (7.18 g, 72.45 mmol, 1.5 eq) in dimethylacetamide (15 mL) was added palladium(II) acetate (542 mg, 2.42 mmol, 0.05 eq) and potassium acetate (9.48 g, 96.61 mmol, 2 eq). The mixture was stirred at 90° C. for 12 h. Water (300 mL) was added, and the mixture was stirred for 1 minute. The aqueous phase was extracted with ethyl acetate (100 mL×3). The combined organic phase was washed with brine (100 mL×2), dried with anhydrous sodium sulfate, filtered, and concentrated in vacuum. The residue was purified by reverse phase C18 column chromatography [ACN/H2O (0.5% FA) from 5% to 50%]. tert-Butyl N-[(1S)-1-[4-(4-methylthiazol-5-yl)phenyl]ethyl]carbamate (9.8 g, 29.85 mmol, 61% yield) was obtained as a gray solid.

### Step 6

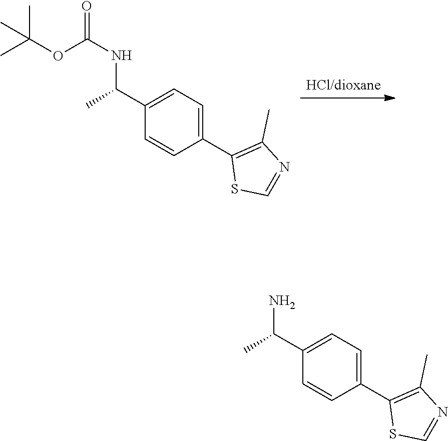 [SMILES: Cc1ncsc1-c1ccc([C@H](C)NC(=O)OC(C)(C)C)cc1 ; Cc1ncsc1-c1ccc([C@H](C)N)cc1]

To a solution of tert-butyl N-[(1S)-1-[4-(4-methylthiazol-5-yl)phenyl]ethyl]carbamate (1.5 g, 4.71 mmol, 1 eq) in dichloromethane (20 mL) was added hydrochloride acid/dioxane (4 M, 20 mL, 17 eq). The mixture was stirred at 25° C. for 12 hours. The reaction mixture was concentrated under reduced pressure to remove dichloromethane. The crude product was triturated with petroleum ether (100 mL). Crude (1S)-1-[4-(4-methylthiazol-5-yl)phenyl]ethanamine hydrochloride (1.1 g) was obtained as a yellow solid.

### Step 7

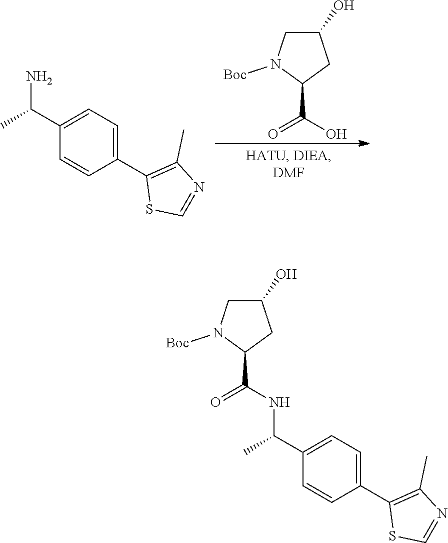 [SMILES: Cc1ncsc1-c1ccc([C@H](C)N)cc1 ; CC(C)(C)OC(=O)N1C[C@H](O)C[C@H]1C(=O)O ; Cc1ncsc1-c1ccc([C@H](C)NC(=O)[C@@H]2C[C@@H](O)CN2C(=O)OC(C)(C)C)cc1]

To a solution of (2S,4R)-1-tert-butoxycarbonyl-4-hydroxy-pyrrolidine-2-carboxylic acid (998 mg, 4.32 mmol, 1.1 eq) and O-(7-azabenzotriazol-1-yl)-N,N,N′,N′-tetramethyluronium hexafluorophosphate (1.79 g, 4.71 mmol, 1.2 eq) in dimethylformamide (10 mL) were added (1S)-1-[4-(4-methylthiazol-5-yl)phenyl]ethanamine hydrochloride (1 g, 3.92 mmol, 1 eq) and diisopropylethyl amine (1.52 g, 11.77 mmol, 2.05 mL, 3 eq). The reaction mixture was stirred at 15° C. for 0.5 hours. The reaction mixture was poured into water (20 mL) and extracted with ethyl acetate (30 mL×3). The combined organic layers were washed with brine (50 mL×3), dried over anhydrous sodium sulfate, filtered, and concentrated under reduced pressure. The residue was purified by silica gel column chromatography (petroleum ether: ethyl acetate from 100:1 to 30:1). tert-Butyl (2S,4R)-4-hydroxy-2-[[(1S)-1-[4-(4-methylthiazol-5-yl)phenyl]ethyl]carbamoyl] pyrrolidine-1-carboxylate (1.2 g, 2.78 mmol, 70% yield) was obtained as a white solid.

### Step 8

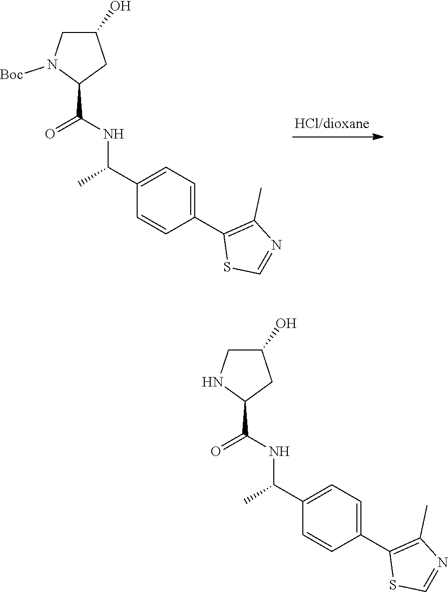 [SMILES: Cc1ncsc1-c1ccc([C@H](C)NC(=O)[C@@H]2C[C@@H](O)CN2C(=O)OC(C)(C)C)cc1 ; Cc1ncsc1-c1ccc([C@H](C)NC(=O)[C@@H]2C[C@@H](O)CN2)cc1]

To a solution of tert-butyl (2S,4R)-4-hydroxy-2-[[(1S)-1-[4-(4-methylthiazol-5-yl) phenyl]ethyl]carbamoyl]pyrrolidine-1-carboxylate (1 g, 2.32 mmol, 1 eq) in dichloromethane (10 mL) was added hydrochloric acid (2.5 M in dioxane, 5 mL, 5.4 eq). The reaction mixture was stirred at 15° C. for 0.5 hours. The reaction mixture was concentrated under reduced pressure. (2S,4R)-4-hydroxy-N-[(1S)-1-[4-(4-methylthiazol-5-yl)phenyl]ethyl]pyrrolidine-2-carboxamide hydrochloride (800 mg, 2.17 mmol, 93% yield) was obtained as a colorless oil.

Methyl 2-[3-[3-(hydroxymethyl)cyclobutoxy]isoxazol-5-yl]-3-methyl-butanoate was converted to (2S,4R)-4-hydroxy-1-((R)-2-(3-((1r,3R)-3-(hydroxymethyl)cyclobutoxy)isoxazol-5-yl)-3-methylbutanoyl)-N—((S)-1-(4-(4-methylthiazol-5-yl)phenyl)ethyl)pyrrolidine-2-carboxamide according to the scheme below using procedures described for other Examples above as well as procedures commonly known to skilled in the art.

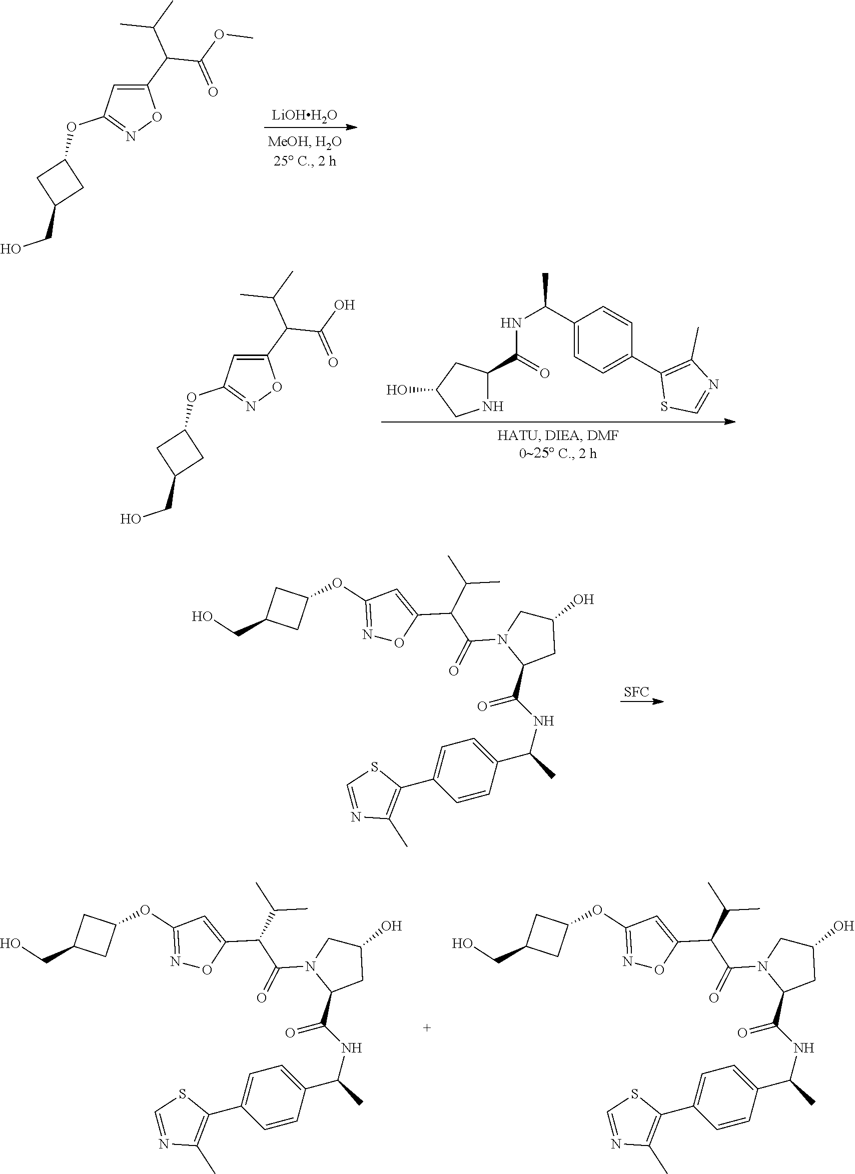

(2S,4R)-4-hydroxy-1-((R)-2-(3-((1r,3R)-3-(hydroxymethyl)cyclobutoxy)isoxazol-5-yl)-3-methylbutanoyl)-N—((S)-1-(4-(4-methylthiazol-5-yl)phenyl)ethyl)pyrrolidine-2-carboxamide was converted to the title compound according to the scheme below.

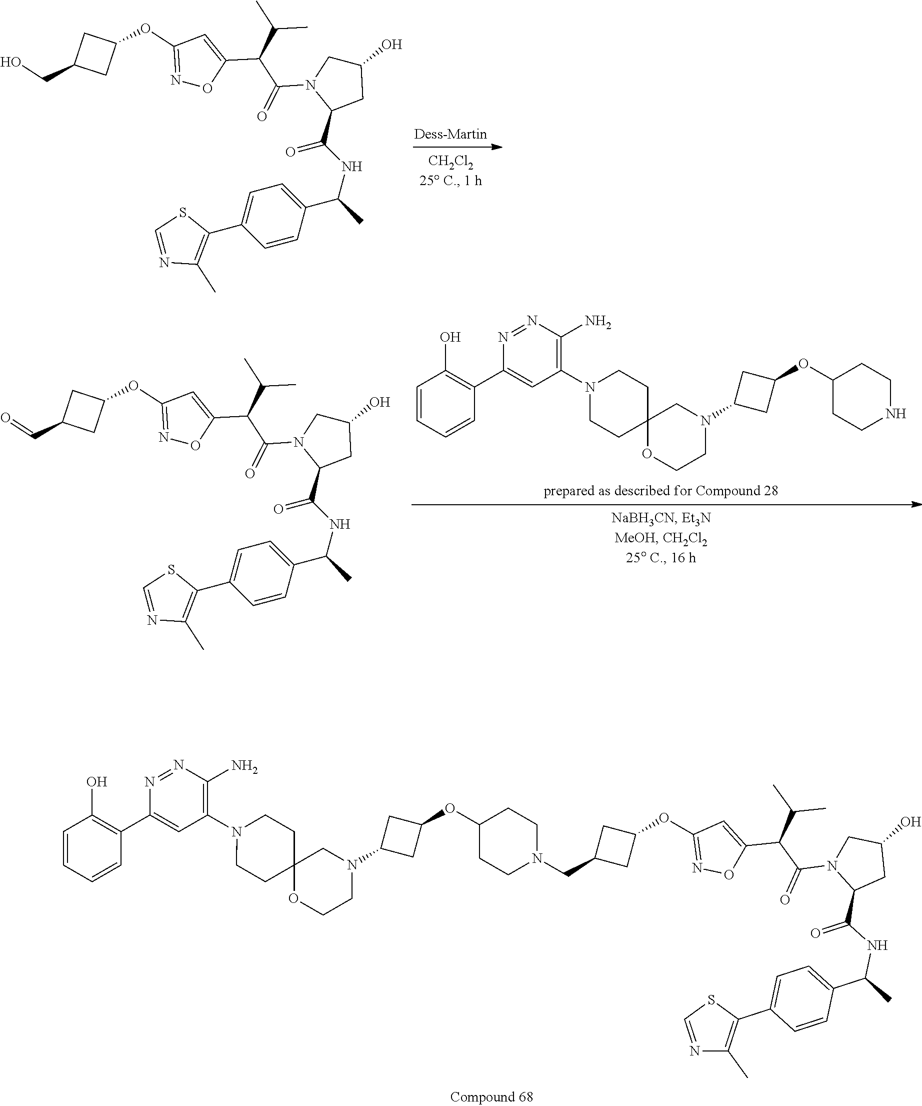

Compound 67 was prepared using analogous procedures.

### Exemplary Synthesis of Compound 69

### Step 1

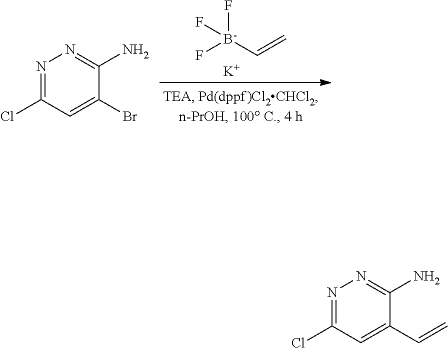

To a solution of 4-bromo-6-chloro-pyridazin-3-amine (5 g, 23.99 mmol, 1 eq) and potassium vinyltrifluoroborate (3.37 g, 25.19 mmol, 1.05 eq) in n-propyl alcohol (50 mL) was added [1,1′-bis(diphenylphosphino)ferrocene]dichloropalladium(II)·dichloromethane complex (1.96 g, 2.40 mmol, 0.1 eq) and triethylamine (7.28 g, 71.96 mmol, 10 mL, 3 eq). The mixture was then degassed and purged with nitrogen 3 times. The mixture was stirred at 100° C. for 4 hours under nitrogen atmosphere. The reaction mixture was diluted with water 200 mL and extracted with ethyl acetate 100 mL (10 mL×3). The combined organic layers were washed with brine 100 mL, dried over anhydrous sodium sulfate, filtered and concentrated under reduced pressure to give a residue. The residue was purified by silica gel chromatography (Petroleum ether/Ethyl acetate=10/1 to 1/1). Compound 6-chloro-4-vinyl-pyridazin-3-amine (1.9 g, 12.17 mmol, 51% yield, 99% purity) was obtained as a yellow solid.

### Step 2

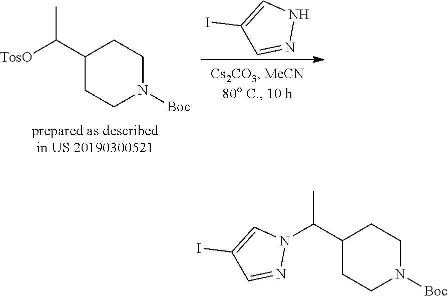

To a solution of 4-iodo-1H-pyrazole (1 g, 5.16 mmol, 1 eq) and tert-butyl 4-[1-(p-tolylsulfonyloxy)ethyl]piperidine-1-carboxylate (2.37 g, 6.19 mmol, 1.2 eq) in acetonitrile (20 mL) was added cesium carbonate (3.36 g, 10.31 mmol, 2 eq). The mixture was stirred at 80° C. for 10 hours. The reaction mixture was diluted with water 100 mL and extracted with ethyl acetate (50 mL×3). The combined organic layers were washed with brine 30 mL, dried over anhydrous sodium sulfate, filtered and concentrated under reduced pressure to give a residue. The residue was purified by prep-HPLC (column: Phenomenex luna C18 (250*70 mm, 15 um); mobile phase: [water (0.225% FA)-ACN]; B %: 50ACN %-80ACN %, 30 min). Compound tert-butyl 4-[1-(4-iodopyrazol-1-yl)ethyl]piperidine-1-carboxylate (1.8 g, 4.40 mmol) was obtained as a colorless oil.

### Step 3

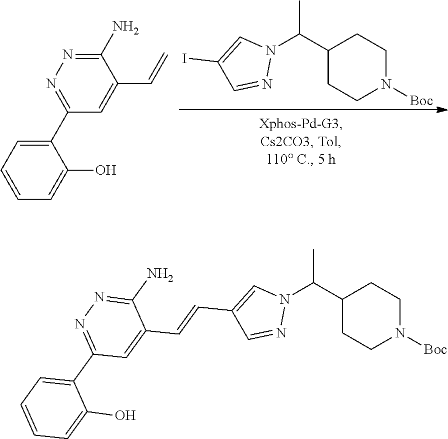

To a solution of 2-(6-amino-5-vinyl-pyridazin-3-yl)phenol (500 mg, 2.34 mmol, 1 eq) and tert-butyl 4-[1-(4-iodopyrazol-1-yl)ethyl]piperidine-1-carboxylate (950.30 mg, 2.34 mmol, 1 eq) in toluene (15 mL) was added (2-dicyclohexylphosphino-2′,4′,6′-triisopropyl-1,1′-biphenyl) [2-(2′-amino-1,1′-biphenyl)]palladium(ii) methanesulfonate (198 mg, 0.23 mmol, 0.1 eq) and cesium carbonate (1.15 g, 3.52 mmol, 1.5 eq). The mixture was stirred at 110° C. for 5 h under nitrogen. The reaction mixture was diluted with water 50 mL and extracted with ethyl acetate (50 mL×3). The combined organic layers were washed with brine 50 mL, dried over anhydrous sodium sulfate, filtered and concentrated under reduced pressure to give a residue. The residue was purified by silica gel column chromatography (petroleum ether/ethyl acetate=10/1 to 0/1). Compound tert-butyl 4-[1-[4-[(E)-2-[3-amino-6-(2-hydroxyphenyl)pyridazin-4-yl]vinyl]pyrazol-1-yl]ethyl]piperidine-1-carboxylate (176 mg, 0.36 mmol) was obtained as a yellow solid.

tert-Butyl 4-[1-[4-[(E)-2-[3-amino-6-(2-hydroxyphenyl)pyridazin-4-yl]vinyl]pyrazol-1-yl]ethyl]piperidine-1-carboxylate was converted to the title compound as described in the scheme below.

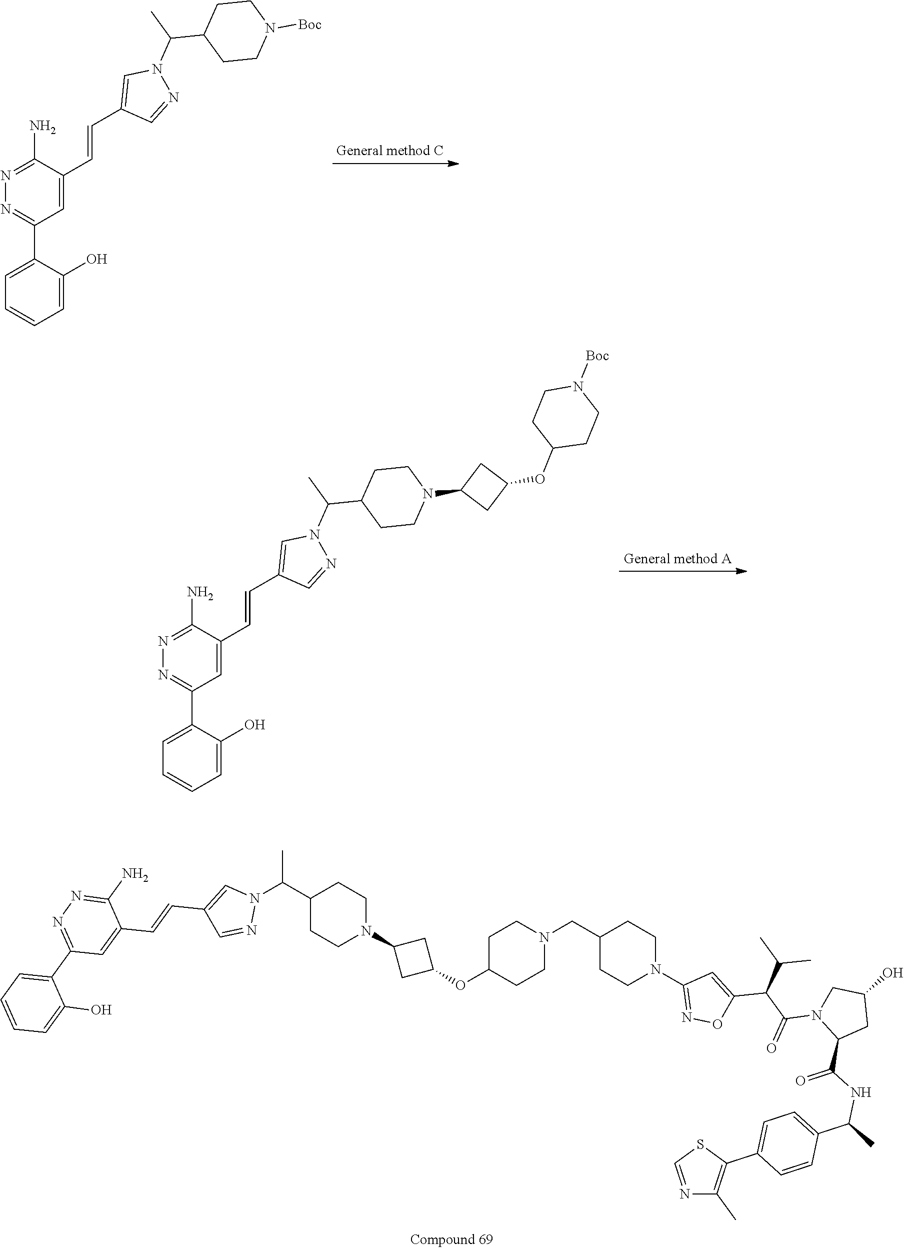 [SMILES: CC(C1CCN(C(=O)OC(C)(C)C)CC1)n1cc(/C=C/c2cc(-c3ccccc3O)nnc2N)cn1 ; CC(C1CCN([C@H]2C[C@H](OC3CCN(C(=O)OC(C)(C)C)CC3)C2)CC1)n1cc(/C=C/c2cc(-c3ccccc3O)nnc2N)cn1 ; Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCC(C(C)n7cc(/C=C/c8cc(-c9ccccc9O)nnc8N)cn7)CC6)C5)CC4)CC3)no2)C(C)C)cc1]

### Exemplary Synthesis of Compounds 70

Prepared according to the schemes below using procedures described or referenced above, as well as general procedures known to those skilled in the art.

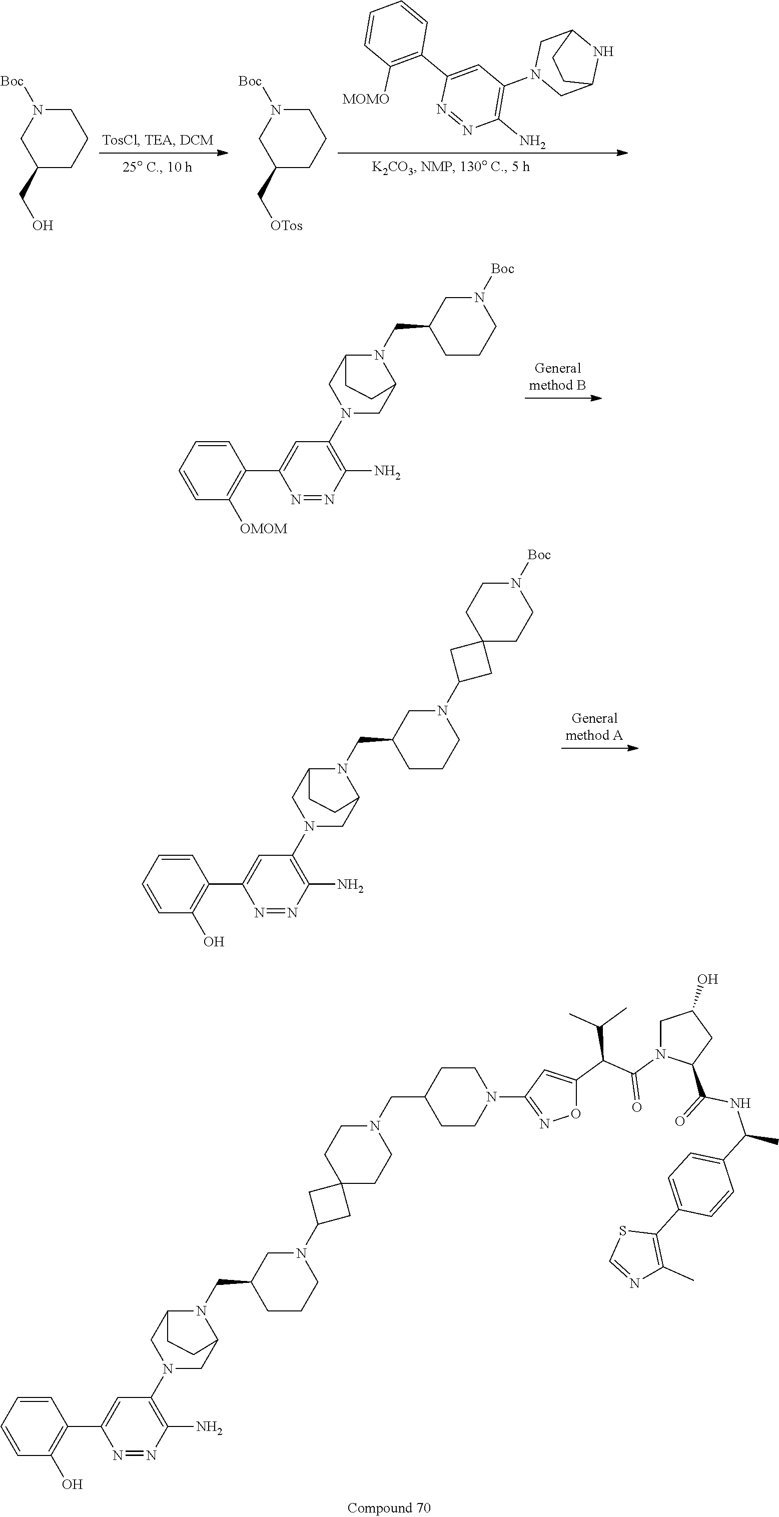

### Exemplary Synthesis of Compound 74

### Step 1

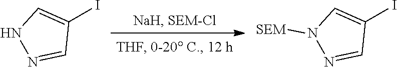 [SMILES: Ic1cn[nH]c1 ; C[Si](C)(C)CCOCn1cc(I)cn1]

To a solution of 4-iodo-1H-pyrazole (10 g, 51.55 mmol, 1 eq) in tetrahydrofuran (100 mL) was added sodium hydride (3.09 g, 77.33 mmol, 60% in mineral oil, 1.5 eq) at 0° C., and the mixture was stirred at 0° C. for 2 hours. To the mixture was added 2-(trimethylsilyl)ethoxy methyl chloride (8.60 g, 51.55 mmol, 9.1 mL, 1 eq) at 0° C. The reaction solution was stirred 20° C. for 12 hours. The reaction mixture was quenched with saturated aqueous solution of ammonium chloride (200 mL), and extracted with ethyl acetate (200 mL×2). The combined organic layer was washed with brine (100 mL×2). The organic layer was dried over anhydrous sodium sulfate, filtered and concentrated under vacuum to get the residue. The residue was purified by silica gel column chromatography (Petroleum ether/Ethyl acetate=1/0 to 10:1) to get the product. 2-[(4-iodopyrazol-1-yl)methoxy]ethyl-trimethyl-silane (14.47 g, 44.63 mmol, 87% yield) was obtained as a yellow oil.

### Step 2

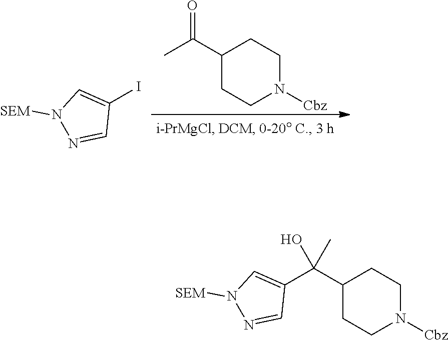

To a solution of 2-[(4-iodopyrazol-1-yl)methoxy]ethyl-trimethyl-silane (9.5 g, 29.30 mmol, 1 eq) in dichloromethane (100 mL) was added isopropylmagnesium chloride (2 M, 22.0 mL, 1.5 eq) dropwise at 0° C., and the solution was stirred at 0° C. for 1 hour. Subsequently, a solution of benzyl 4-acetylpiperidine-1-carboxylate (9.19 g, 35.16 mmol, 1.2 eq) in dichloromethane (100 mL) was added at 0° C. The reaction solution was stirred at 20° C. for 2 hours. The reaction solution was quenched with saturated aqueous ammonium chloride (100 mL) and extracted with dichloromethane (100 mL×2). The combined organic layer was dried over anhydrous sodium sulfate, filtered and concentrated under vacuum. The residue was purified by silica gel column chromatography (Petroleum ether/Ethyl acetate=3/1 to 1/2). Benzyl 4-[1-hydroxy-1-[1-(2-trimethylsilylethoxymethyl) pyrazol-4-yl]ethyl]piperidine-1-carboxylate (11.7 g) was obtained as a colorless gum.

### Step 3

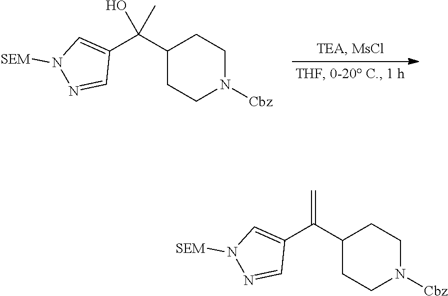 [SMILES: CC(O)(c1cnn(COCC[Si](C)(C)C)c1)C1CCN(C(=O)OCc2ccccc2)CC1 ; C=C(c1cnn(COCC[Si](C)(C)C)c1)C1CCN(C(=O)OCc2ccccc2)CC1]

To a solution of benzyl 4-[1-hydroxy-1-[1-(2-trimethylsilylethoxymethyl)pyrazol-4-yl]ethyl] piperidine-1-carboxylate (9.2 g, 20.02 mmol, 1 eq) and triethylamine (6.08 g, 60.05 mmol, 8.4 mL, 3 eq) in tetrahydrofuran (80 mL) was added methanesulfonyl chloride (5.73 g, 50.04 mmol, 3.9 mL, 2.5 eq) dropwise at 0° C., and the resulting solution was stirred 20° C. for 1 hour. The reaction solution was quenched with saturated aqueous ammonium chloride (20 mL) and extracted with ethyl acetate (20 mL×2). The combined organic layer was dried over anhydrous sodium sulfate, filtered and concentrated under vacuum. The residue was purified by silica gel column chromatography (Petroleum ether/Ethyl acetate=10/1 to 6/1). Benzyl 4-[1-[1-(2-trimethylsilylethoxymethyl)pyrazol-4-yl]vinyl]piperidine-1-carboxylate (5.77 g, 13.07 mmol, 65% yield) was obtained as a colorless gum.

The title compound was prepared according to the schemes below using procedures described or referenced above, as well as general procedures known to those skilled in the art.

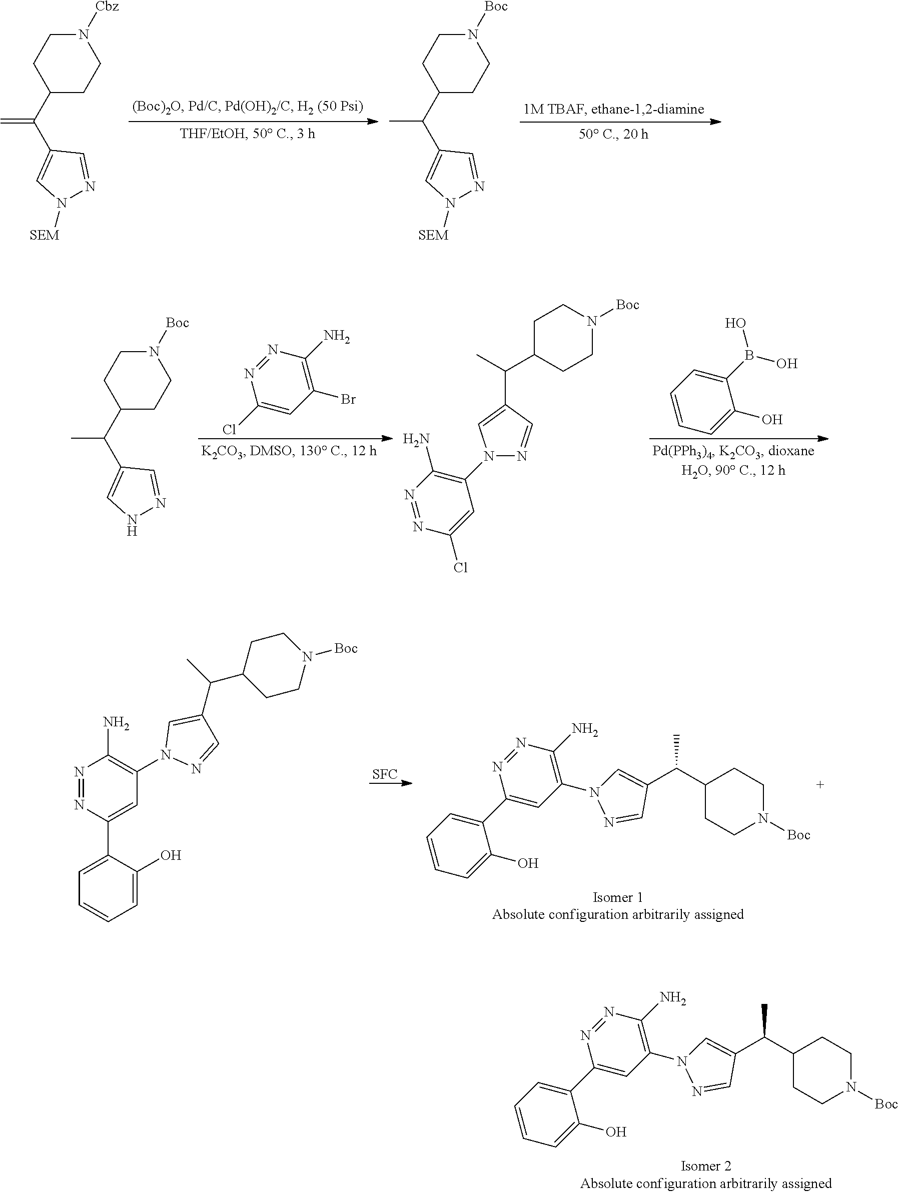

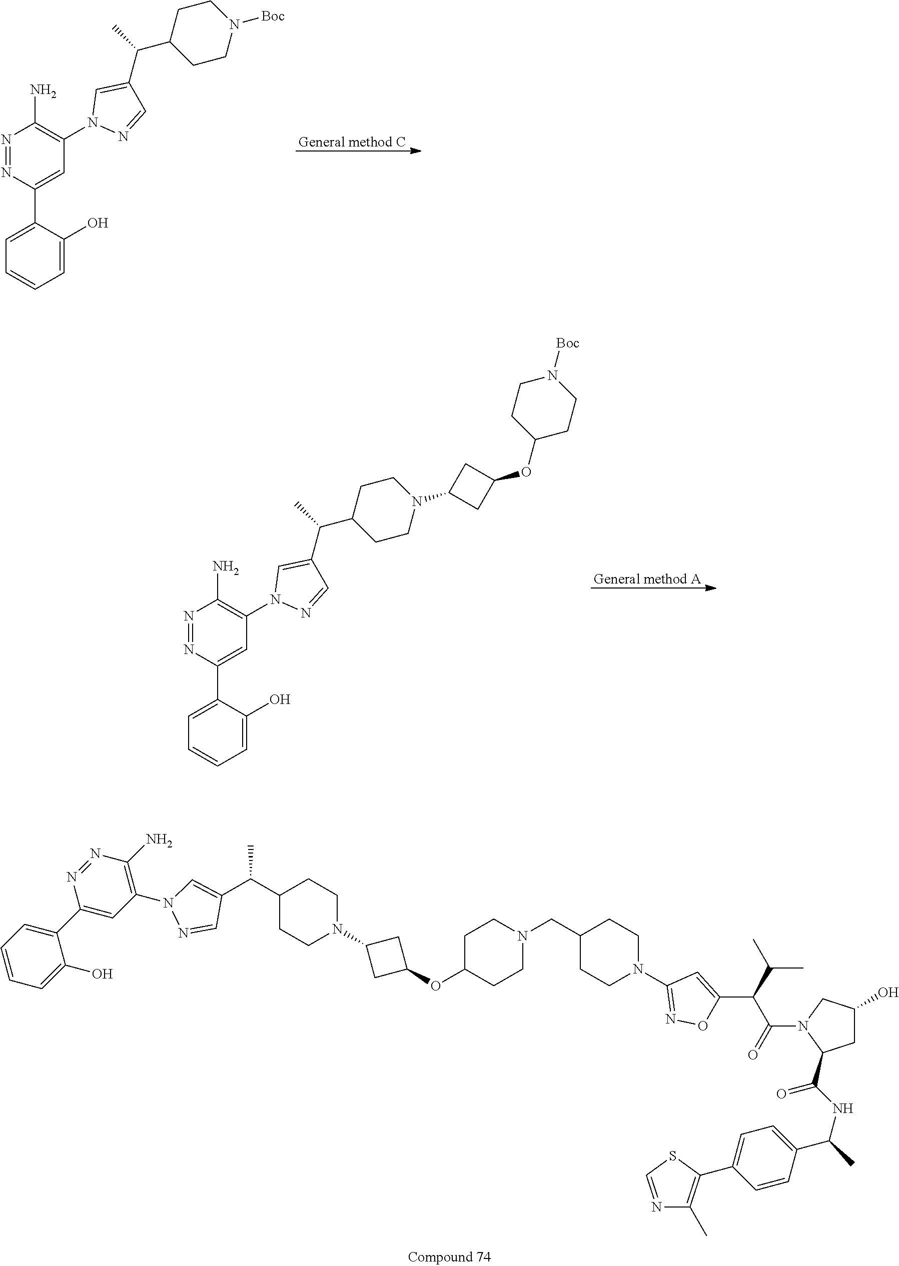 [SMILES: C[C@@H](c1cnn(-c2cc(-c3ccccc3O)nnc2N)c1)C1CCN(C(=O)OC(C)(C)C)CC1 ; "General method C" ; C[C@@H](c1cnn(-c2cc(-c3ccccc3O)nnc2N)c1)C1CCN([C@H]2C[C@H](OC3CCN(C(=O)OC(C)(C)C)CC3)C2)CC1 ; "General method A" ; Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCC([C@@H](C)c7cnn(-c8cc(-c9ccccc9O)nnc8N)c7)CC6)C5)CC4)CC3)no2)C(C)C)cc1]

Compound 75 was prepared using analogous procedures.

### Exemplary Synthesis of Compound 76

4-(3-Formylcyclobutoxy)piperidine-1-carboxylate was prepared according to the scheme below using procedures described above as well as procedures commonly known to those skilled in the art.

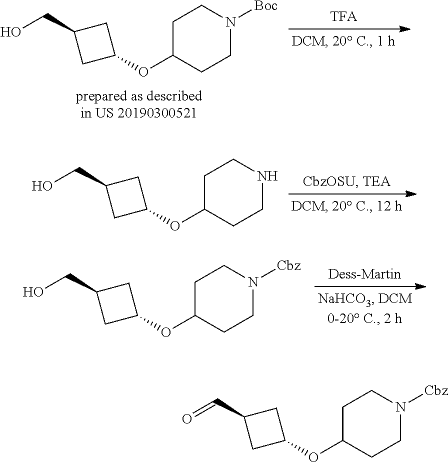

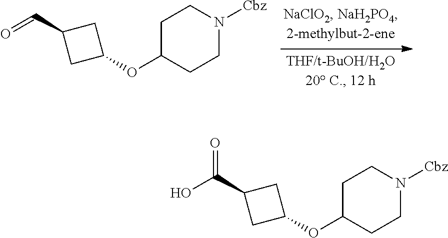 [SMILES: O=C[C@H]1C[C@H](OC2CCN(C(=O)OCc3ccccc3)CC2)C1 ; O=C(OCc1ccccc1)N1CCC(O[C@H]2C[C@H](C(=O)O)C2)CC1]

To a solution of benzyl 4-(3-formylcyclobutoxy)piperidine-1-carboxylate (300 mg, 0.94 mmol, 1 eq) in tert-butyl alcohol (2 mL), tetrahydrofuran (2 mL) and water (2 mL) was added sodium dihydrogen phosphate (567 mg, 4.73 mmol, 5 eq), sodium chlorite (256 mg, 2.84 mmol, 3 eq), and 2-methylbut-2-ene (663 mg, 9.45 mmol, 1.0 mL, 10 eq). The reaction mixture was stirred at 20° C. for 12 hours. The reaction mixture was concentrated under vacuum to remove most solvents, diluted with water (6 mL) and extracted with ethyl acetate (20 mL). The organic layer was washed with brine (10 mL), dried over anhydrous sodium sulfate, filtered and concentrated under vacuum to get the crude product. The crude 3-[(1-benzyloxycarbonyl-4-piperidyl)oxy] cyclobutanecarboxylic acid (320 mg) was obtained as a colorless gum and used directly without purification.

3-[(1-benzyloxycarbonyl-4-piperidyl)oxy] cyclobutanecarboxylic acid was converted to the title compound as shown in the scheme below.

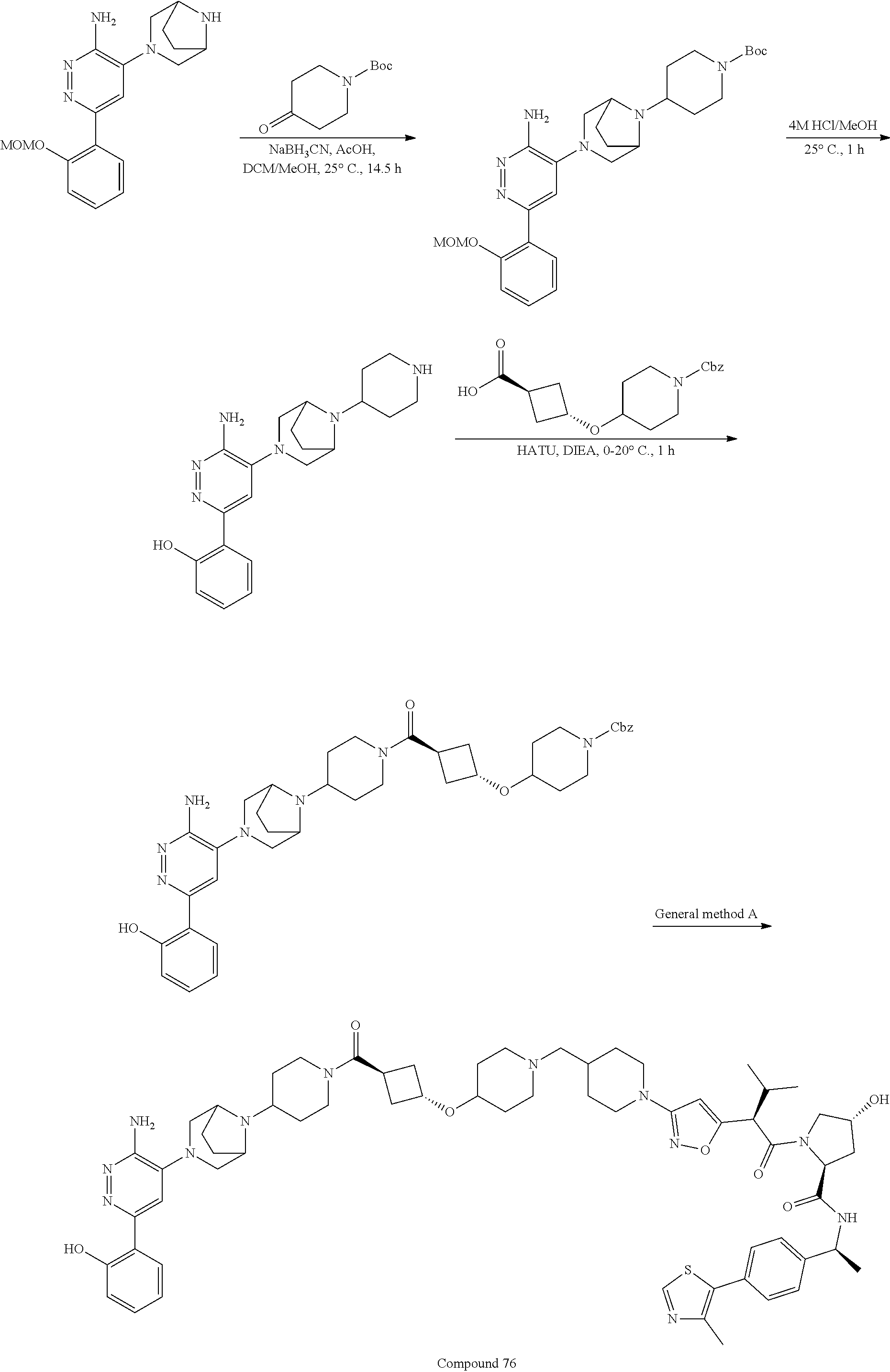

### Exemplary Synthesis of Compound 77

Prepared according to the scheme below using procedures described above, as well as general procedures known to those skilled in the art.

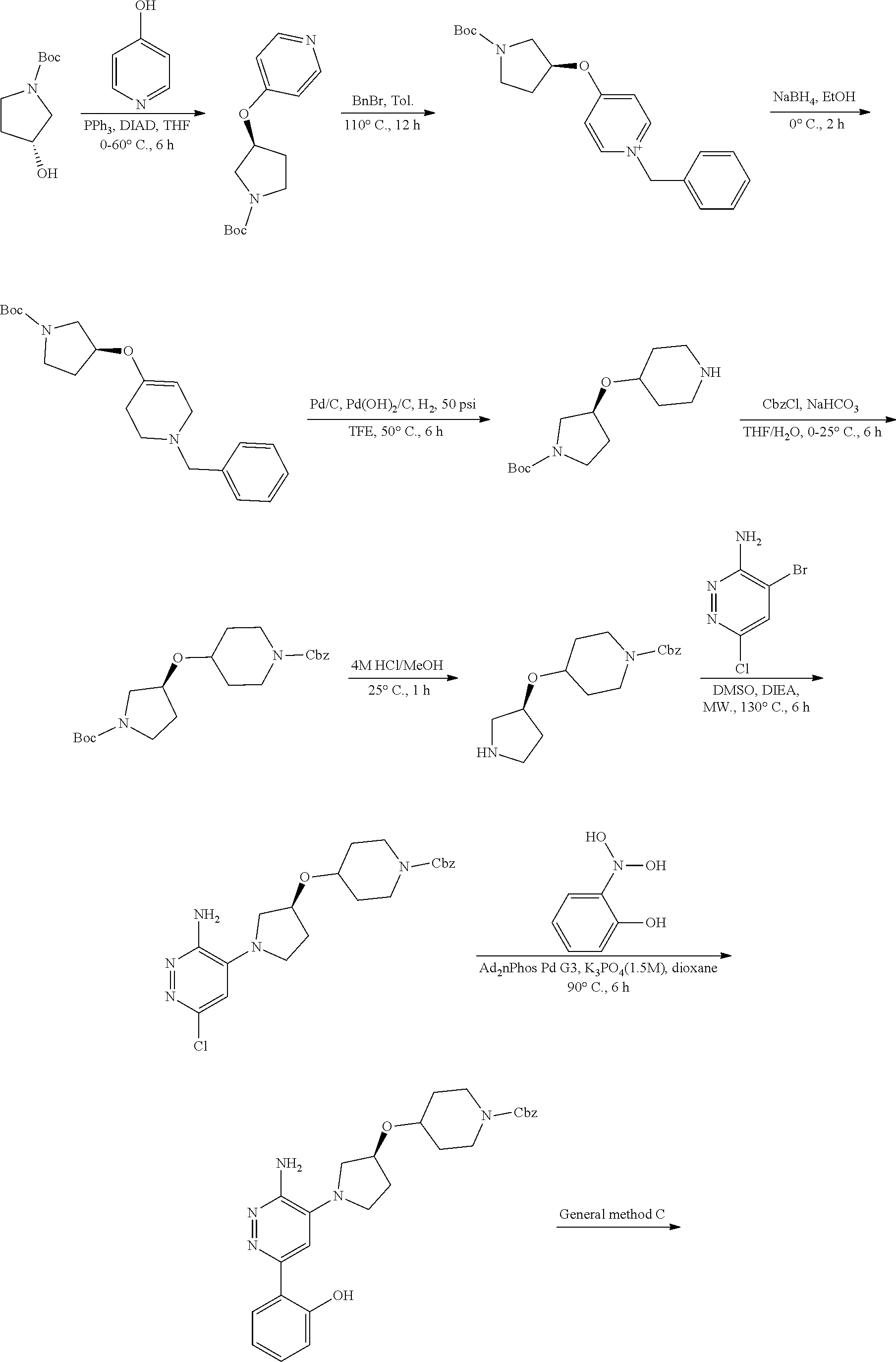

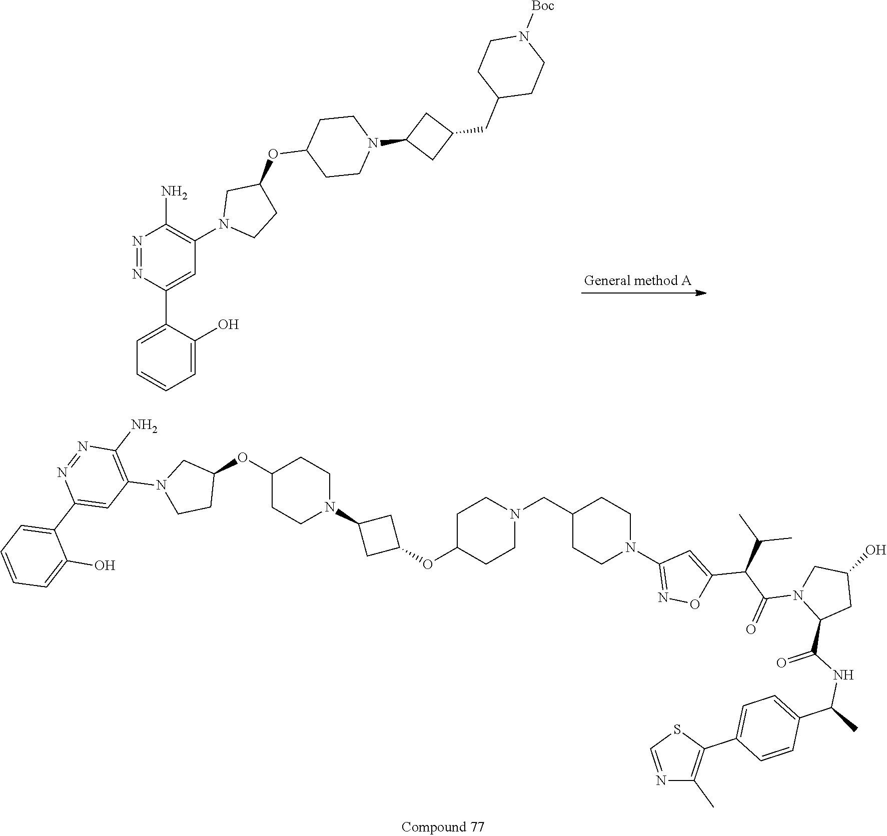

### Exemplary Synthesis of Compound 80

Prepared according to the schemes below using procedures described above, as well as general procedures commonly known to those skilled in the art.

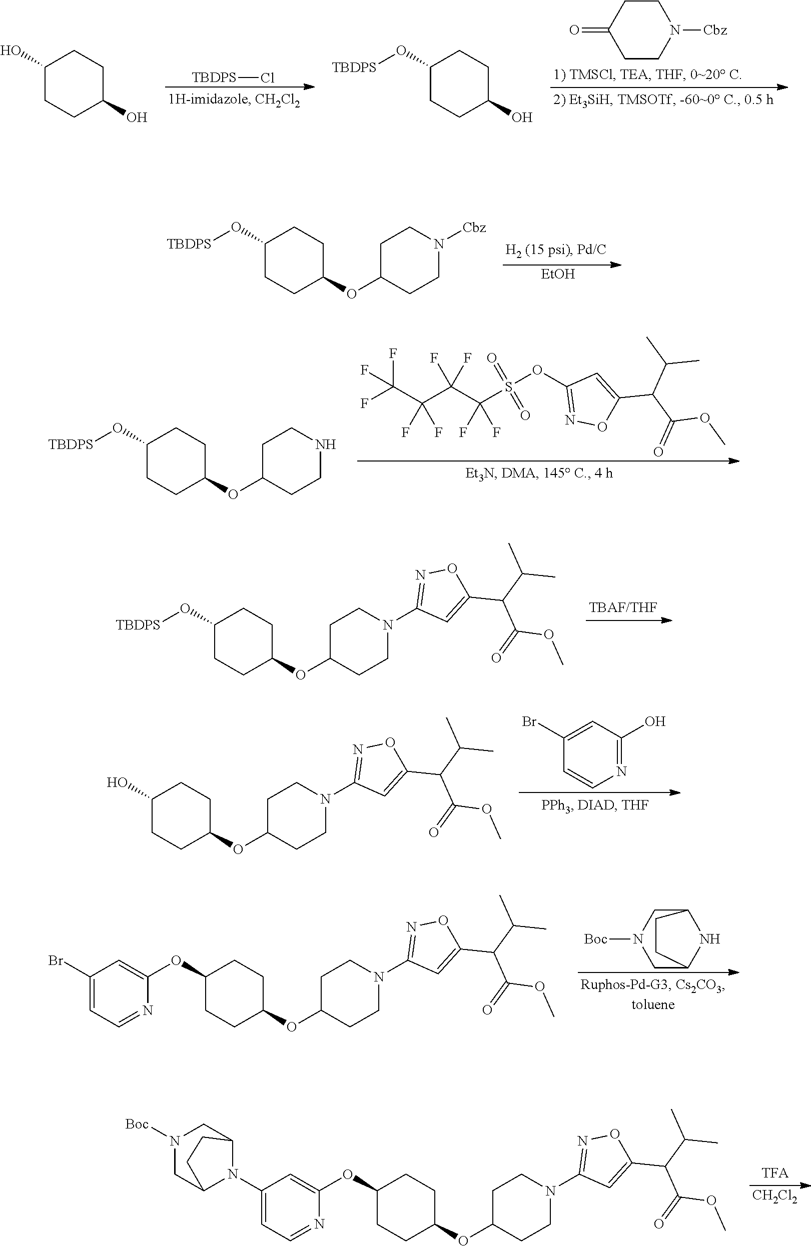

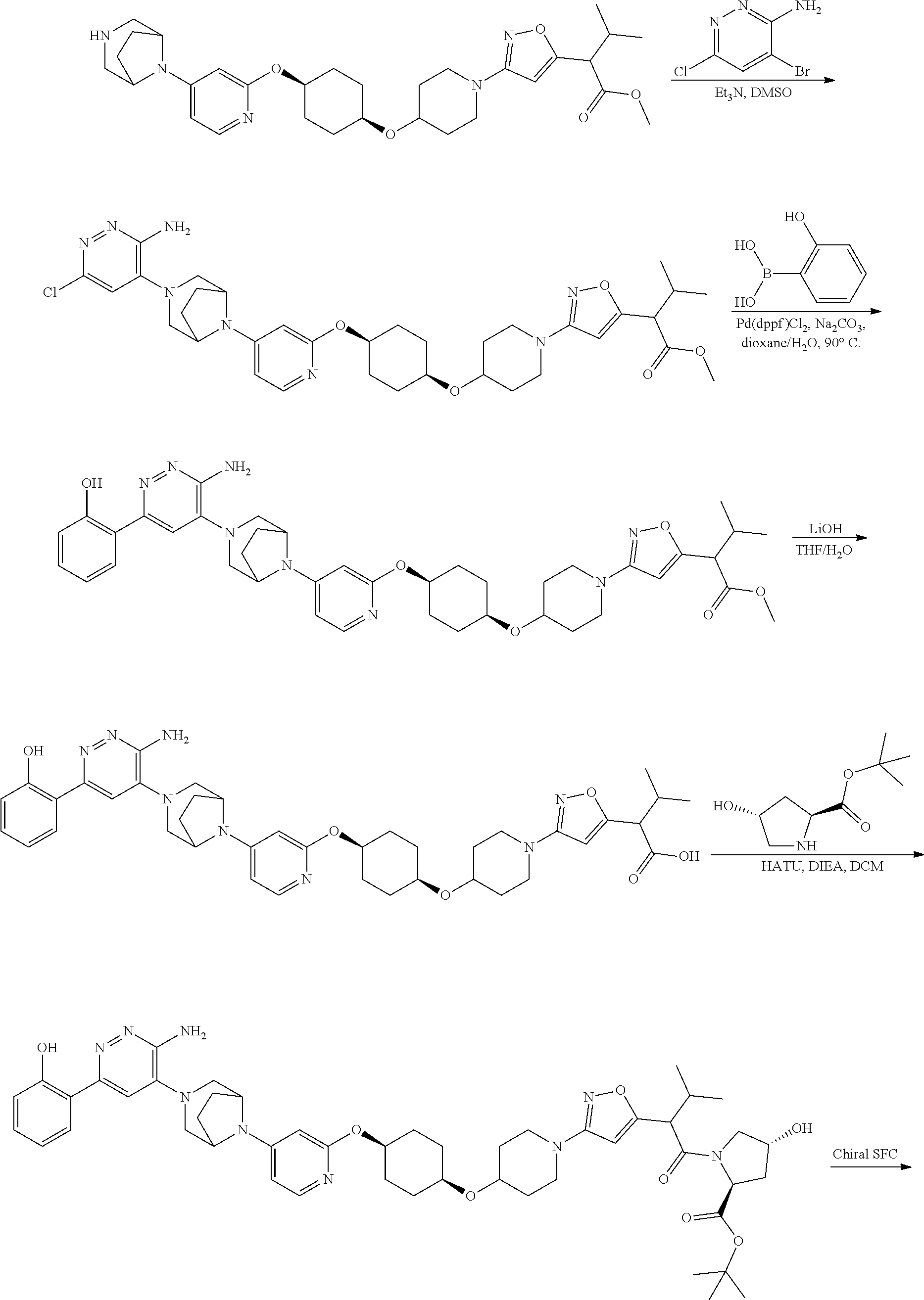

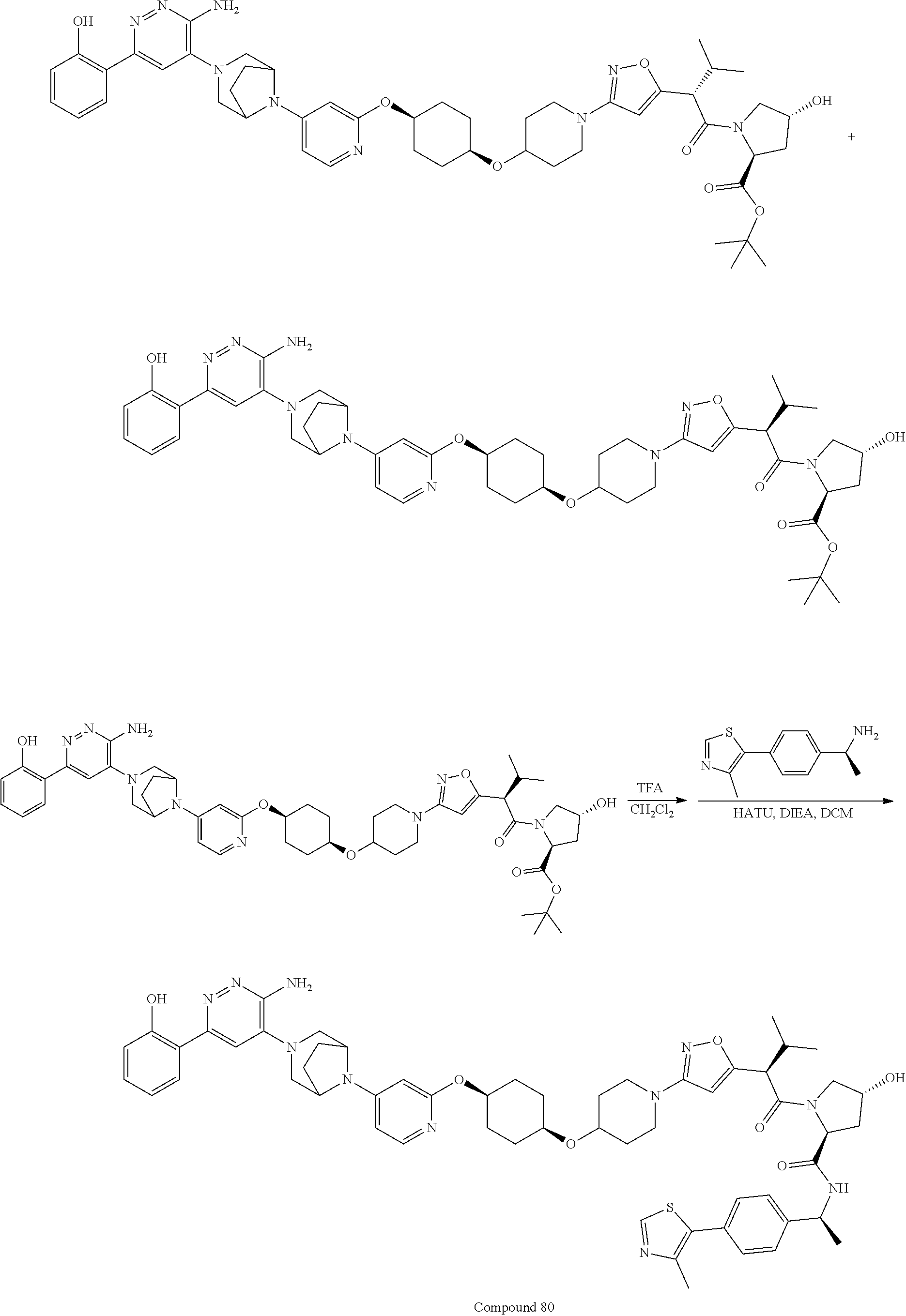

Compound 79 was prepared using analogous procedures.

### Exemplary Synthesis of Compound 84

### Step 1

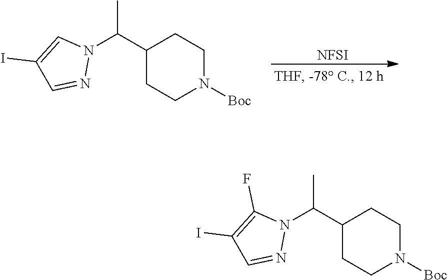 [SMILES: CC(C1CCN(C(=O)OC(C)(C)C)CC1)n1cc(I)cn1 ; "NFSI" ; CC(C1CCN(C(=O)OC(C)(C)C)CC1)n1ncc(I)c1F]

To a solution of tert-butyl 4-[1-(4-iodopyrazol-1-yl)ethyl]piperidine-1-carboxylate (10 g, 24.67 mmol, 1 eq) in tetrahydrofuran (150 mL) was added lithium diisopropylamide (2 M, 24.67 mL, 2 eq) at −78° C., and the mixture was then stirred at −78° C. for 30 minutes. To the reaction mixture was added N-(benzenesulfonyl)-N-fluoro-benzenesulfonamide (23.34 g, 74.02 mmol, 3 eq) in tetrahydrofuran (50 mL) at −78° C., and the mixture was stirred at −78° C. for 11.5 hours. 200 mL of water was added, and the mixture extracted with ethyl acetate (50 mL×3). The combined organic phase was washed with brine (30 mL×2), dried with anhydrous sodium sulfate, filtered and concentrated in vacuum. The residue was purified by prep-HPLC (column: Phenomenex luna C18 250*80 mm*10 um; mobile phase: [water (0.1% TFA)-ACN]; B %: 45%-75%, 22 min). Compound tert-Butyl 4-[1-(5-fluoro-4-iodo-pyrazol-1-yl)ethyl]piperidine-1-carboxylate (2.4 g, 5.67 mmol, 23% yield) was obtained as a colorless oil.

The title compound was prepared according to the scheme below using procedures described above, as well as general procedures known to those skilled in the art.

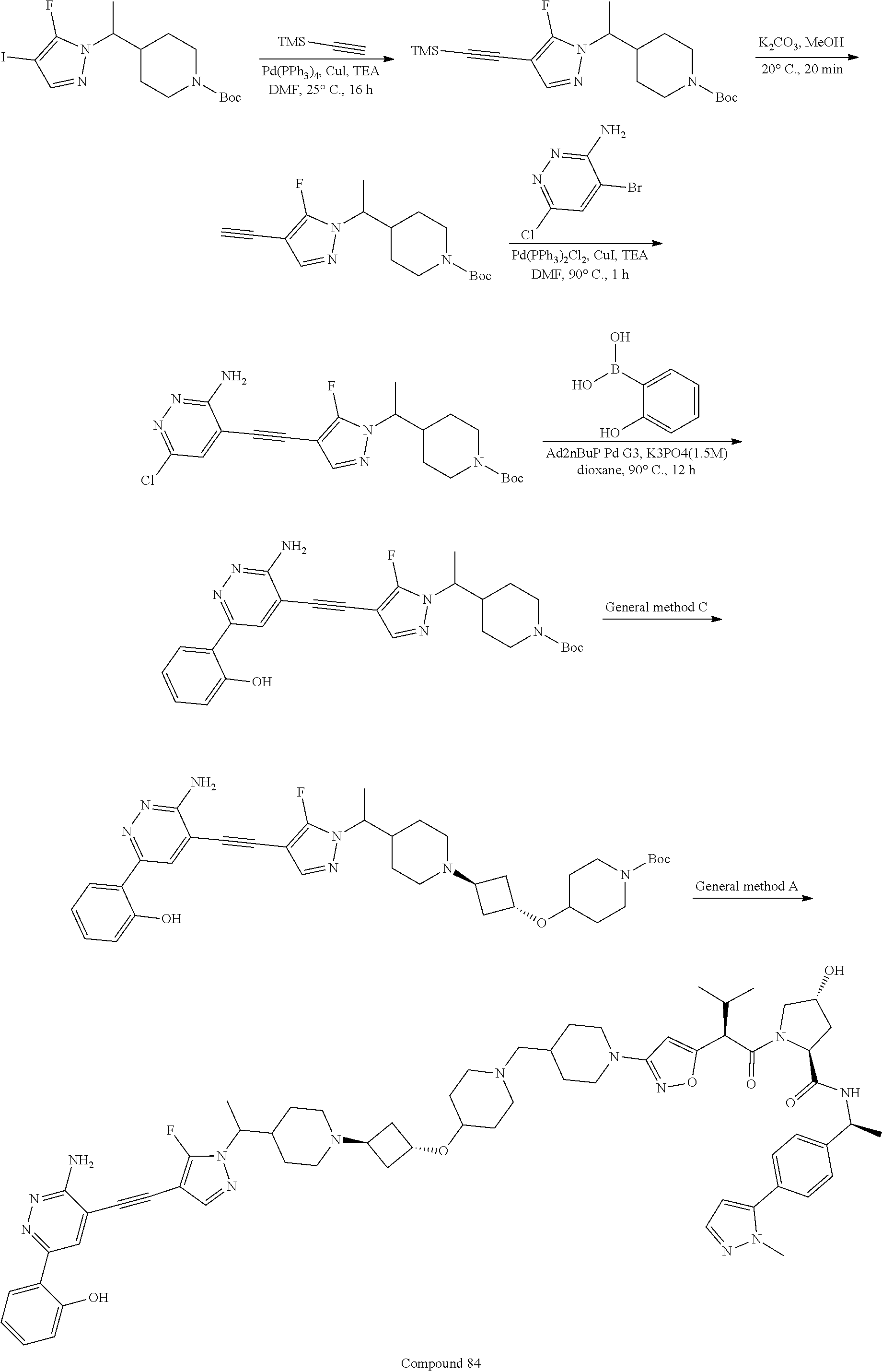

Compound 100 was prepared using analogous procedures.

### Exemplary Synthesis of Compound 85

Prepared according to the scheme below using procedures described above, as well as general procedures known to those skilled in the art.

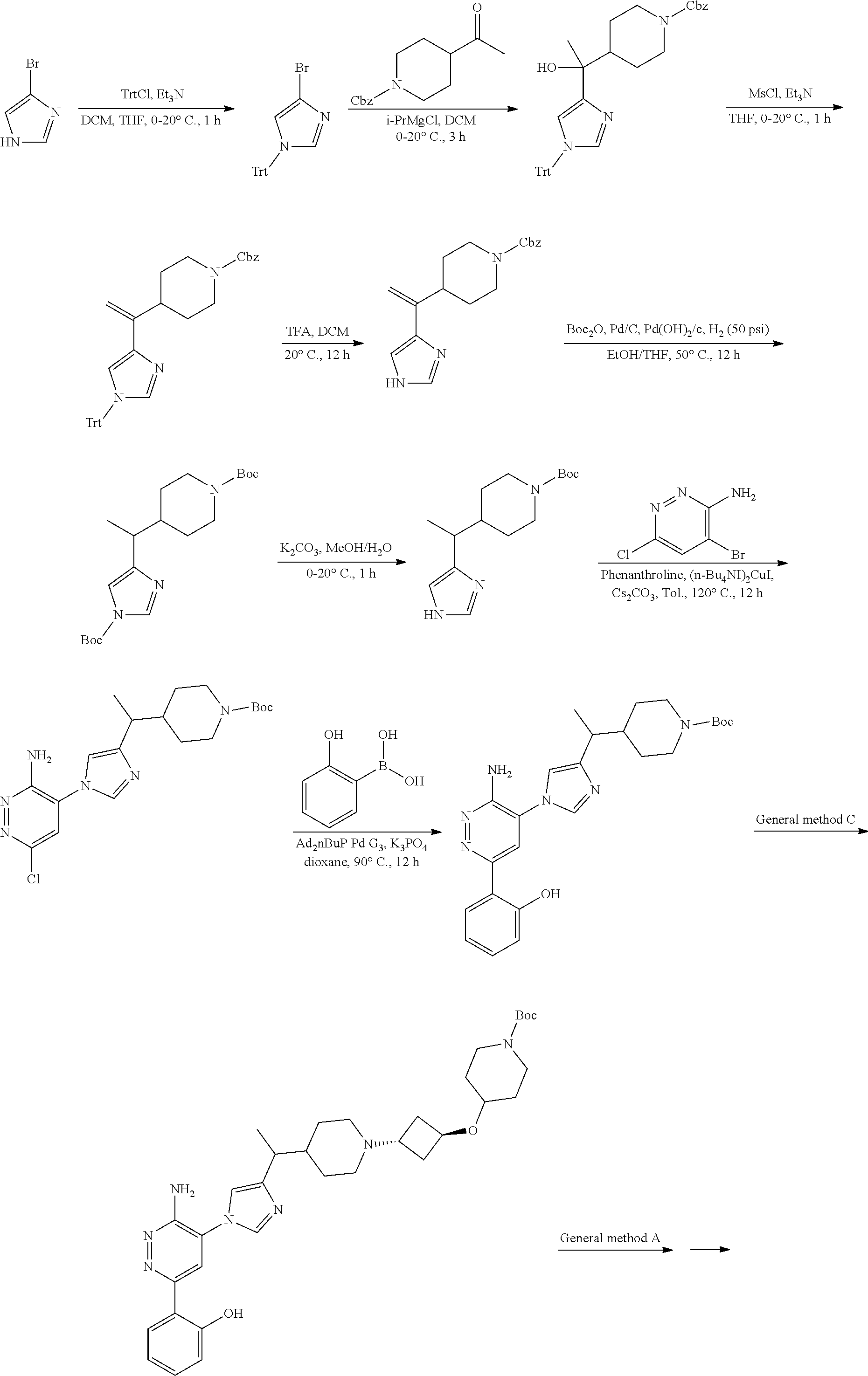

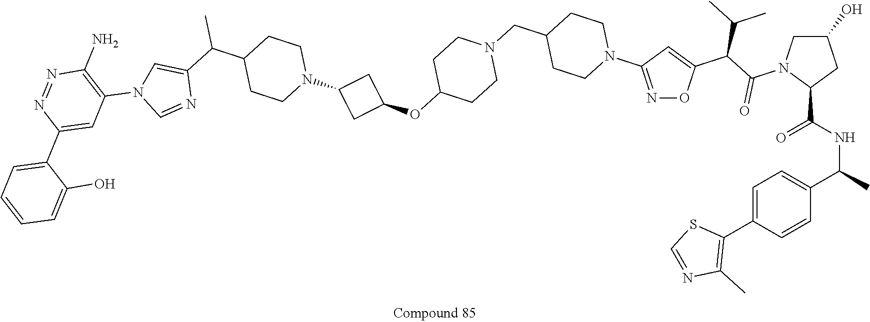 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCC(C(C)c7cn(-c8cc(-c9ccccc9O)nnc8N)cn7)CC6)C5)CC4)CC3)no2)C(C)C)cc1]

### Exemplary Synthesis of Compound 87

The title compound was prepared according to the scheme below using procedures described or referenced above, as well as general procedures known to those skilled in the art.

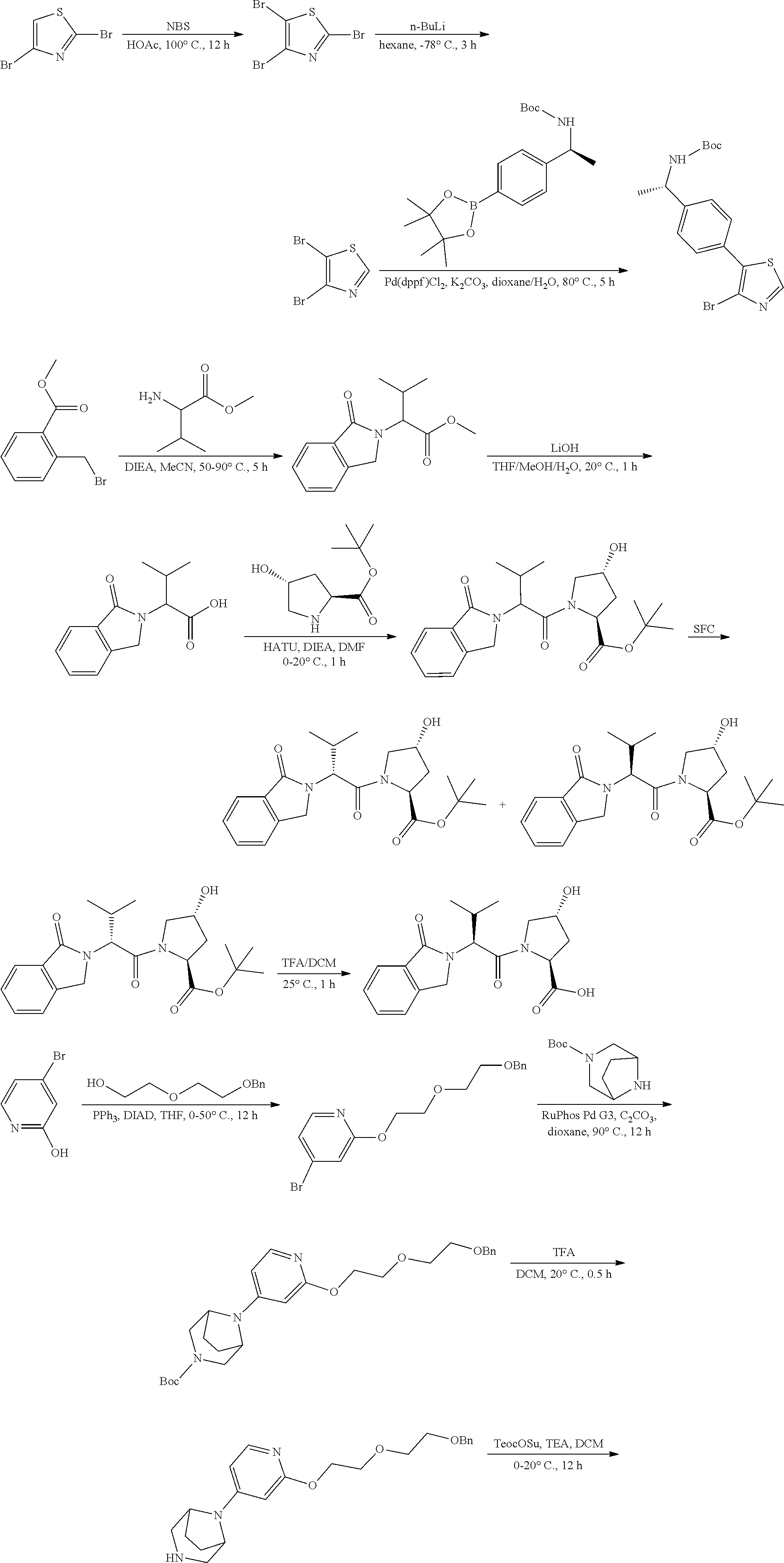

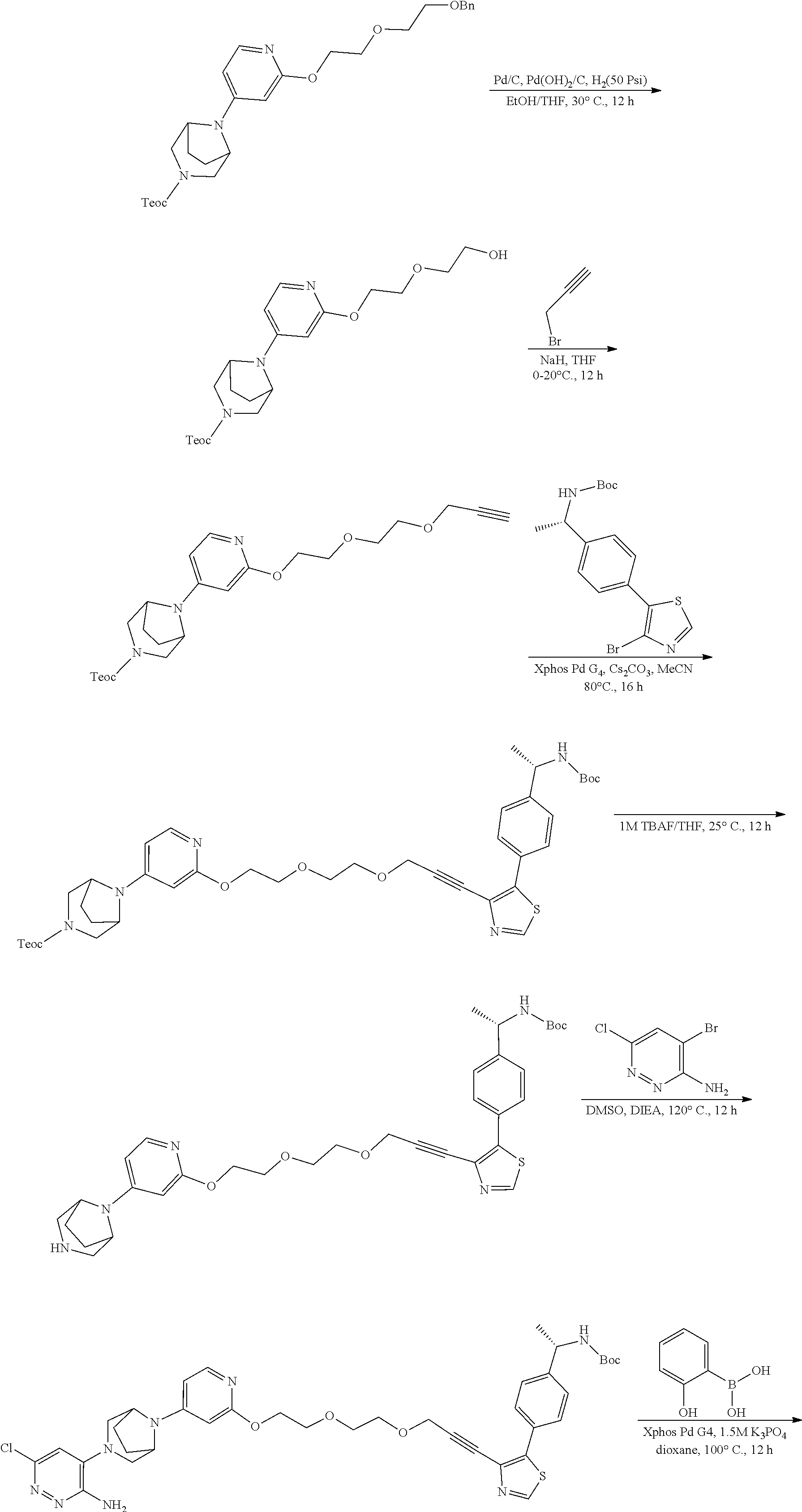

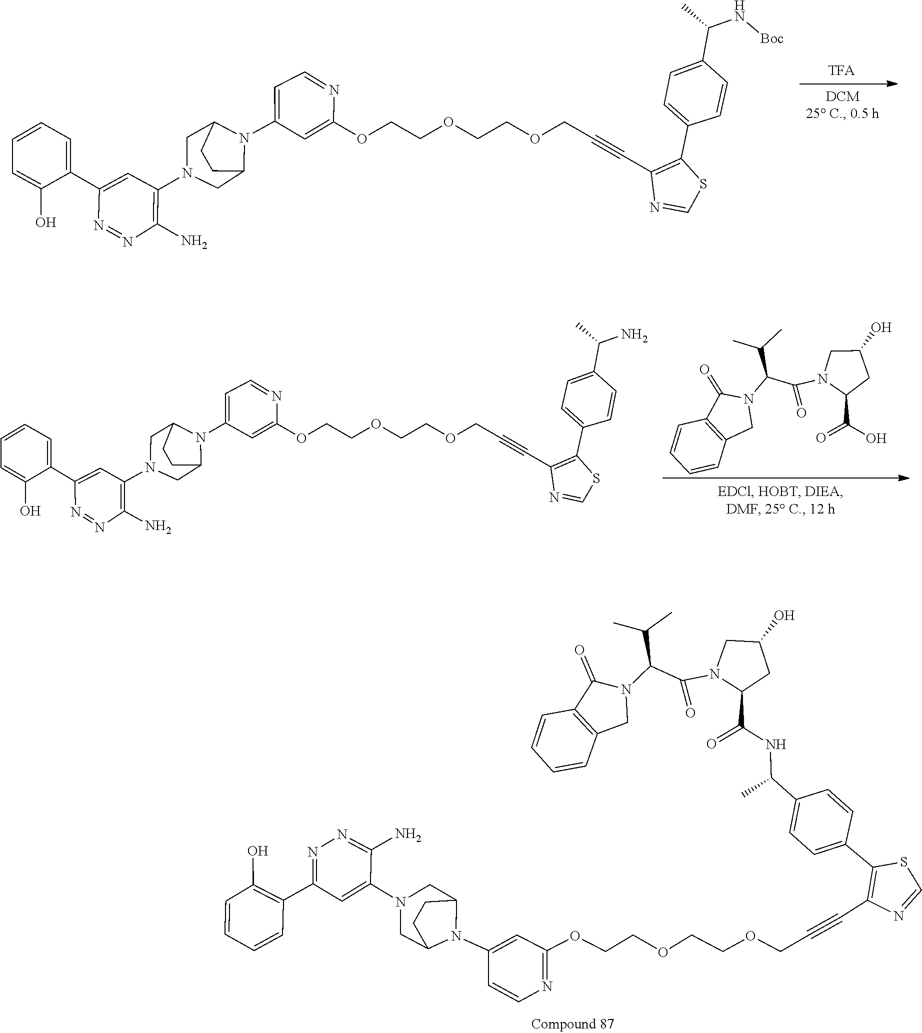 [SMILES: C[C@H](NC(=O)OC(C)(C)C)c1ccc(-c2scnc2C#CCOCCOCCOc2cc(N3C4CCC3CN(c3cc(-c5ccccc5O)nnc3N)C4)ccn2)cc1 ; C[C@H](N)c1ccc(-c2scnc2C#CCOCCOCCOc2cc(N3C4CCC3CN(c3cc(-c5ccccc5O)nnc3N)C4)ccn2)cc1 ; CC(C)[C@@H](C(=O)N1C[C@H](O)C[C@H]1C(=O)O)N1Cc2ccccc2C1=O ; CC(C)[C@@H](C(=O)N1C[C@H](O)C[C@H]1C(=O)N[C@@H](C)c1ccc(-c2scnc2C#CCOCCOCCOc2cc(N3C4CCC3CN(c3cc(-c5ccccc5O)nnc3N)C4)ccn2)cc1)N1Cc2ccccc2C1=O]

Compounds 72, 82, 83, and 86 were prepared using analogous procedures.

### Exemplary Synthesis of Compound 88

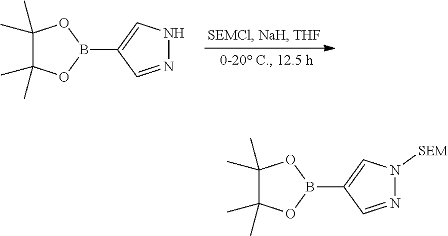 [SMILES: CC1(C)OB(c2cn[nH]c2)OC1(C)C ; CC1(C)OB(c2cnn(COCC[Si](C)(C)C)c2)OC1(C)C]

To a solution of 4-(4,4,5,5-tetramethyl-1,3,2-dioxaborolan-2-yl)-1H-pyrazole (20 g, 103.07 mmol, 1 eq) in tetrahydrofuran (200 mL) was added sodium hydride (6.18 g, 154.61 mmol, 60% purity in mineral oil, 1.5 eq) at 0° C. The reaction mixture was stirred at 0° C. for 0.5 hours. Then 2-(trimethylsilyl)ethoxymethyl chloride (20.62 g, 123.69 mmol, 21.9 mL, 1.2 eq) in tetrahydrofuran (50 mL) was added to the mixture, and the reaction mixture was stirred at 20° C. for 12 hours. Saturated ammonium chloride (200 mL) was added to quench the reaction. The aqueous phase was extracted with ethyl acetate (300 mL×3). The combined organic phase was washed with brine (300 mL), dried over sodium sulfate, filtered and concentrated in vacuum. The residue was purified by silica gel chromatography (petroleum ether: ethyl acetate=1:0 to 1:1). Trimethyl-[2-[[4-(4,4,5,5-tetramethyl-1,3,2-dioxaborolan-2-yl)pyrazo]-1-yl]methoxy]ethyl]silane (30 g, 92.51 mmol, 89% yield) was obtained as a light yellow oil.

Trimethyl-[2-[[4-(4,4,5,5-tetramethyl-1,3,2-dioxaborolan-2-yl)pyrazo]-1-yl]methoxy]ethyl]silane was converted to 6-(2-(methoxymethoxy)phenyl)-4-(1-((2-(trimethylsilyl)ethoxy)methyl)-1H-pyrazol-4-yl)pyridazin-3-amine as shown in the scheme below using procedures described above and commonly known to those skilled in the art.

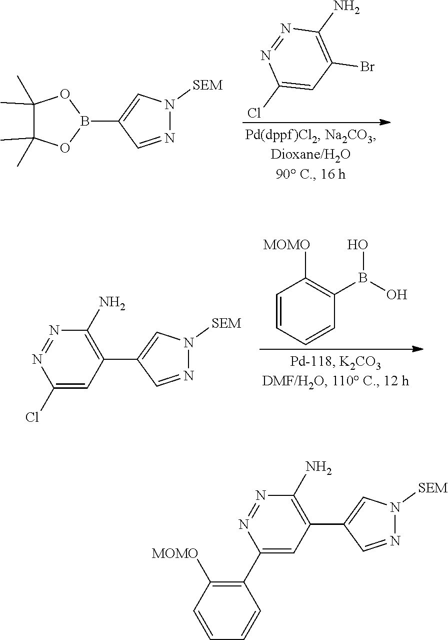

6-(2-(methoxymethoxy)phenyl)-4-(1-((2-(trimethylsilyl)ethoxy)methyl)-1H-pyrazol-4-yl)pyridazin-3-amine as shown in the scheme below using procedures described above as well as those commonly known to skilled in the art.

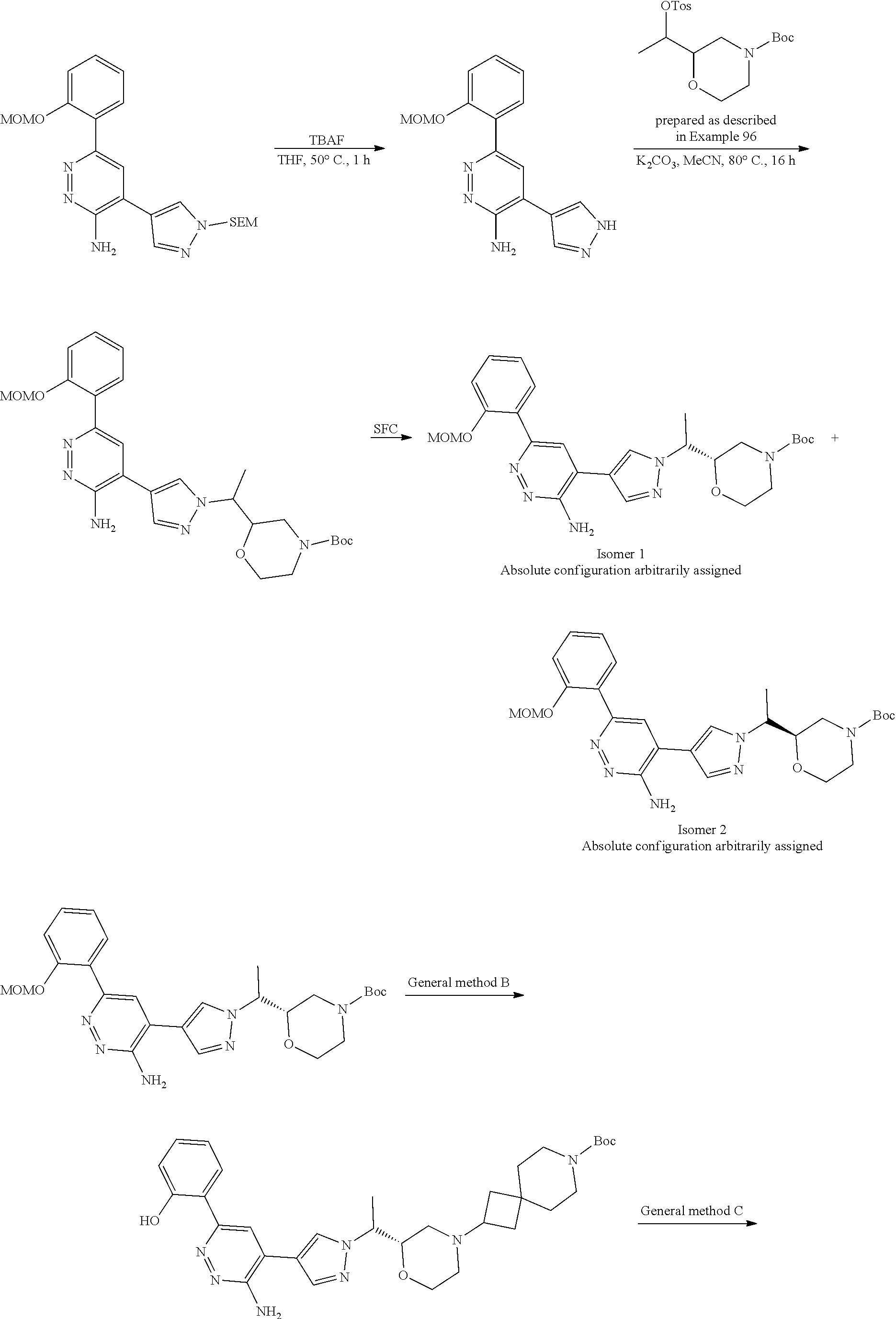

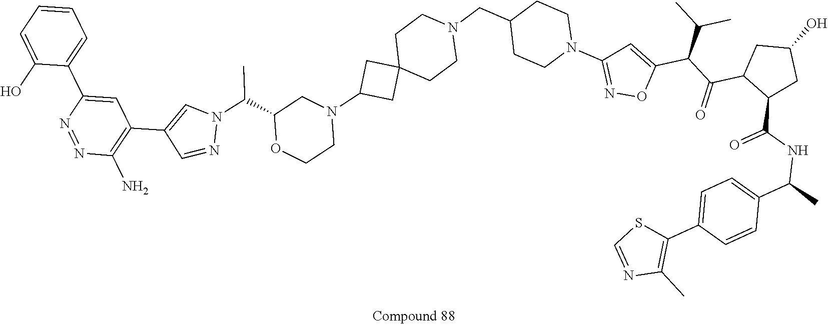 [SMILES: Cc1ncsc1-c1ccc([C@H](C)NC(=O)[C@@H]2C[C@@H](O)CC2C(=O)[C@@H](c2cc(N3CCC(CN4CCC5(CC4)CC(N4CCO[C@H](C(C)n6cc(-c7cc(-c8ccccc8O)nnc7N)cn6)C4)C5)CC3)no2)C(C)C)cc1]

Exemplary Compound 89 was prepared using analogous procedures.

### Exemplary Synthesis of Compound 91

Prepared according to the scheme below using procedures described for other Examples above.

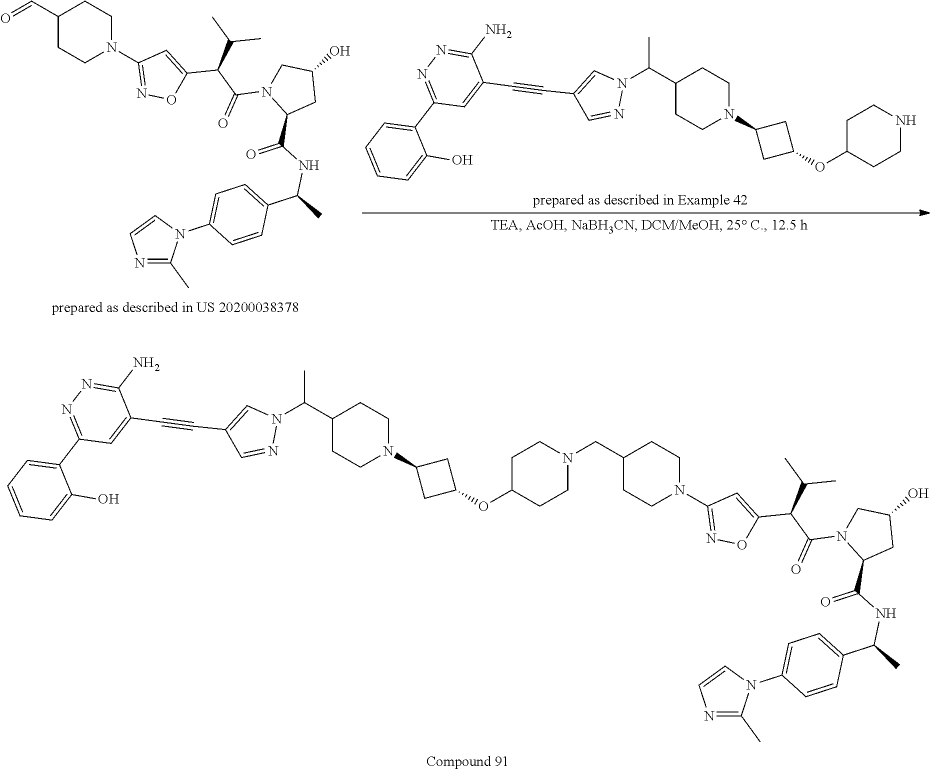 [SMILES: Cc1nccn1-c1ccc([C@H](C)NC(=O)[C@@H]2C[C@@H](O)CN2C(=O)[C@@H](c2cc(N3CCC(C=O)CC3)no2)C(C)C)cc1 ; CC(C1CCN([C@H]2C[C@H](OC3CCNCC3)C2)CC1)n1cc(C#Cc2cc(-c3ccccc3O)nnc2N)cn1 ; Cc1nccn1-c1ccc([C@H](C)NC(=O)[C@@H]2C[C@@H](O)CN2C(=O)[C@@H](c2cc(N3CCC(CN4CCC(O[C@H]5C[C@H](N6CCC(C(C)n7cc(C#Cc8cc(-c9ccccc9O)nnc8N)cn7)CC6)C5)CC4)CC3)no2)C(C)C)cc1]

Compound 90 was prepared using analogous procedures.

### Exemplary Synthesis of Compound 94

### Step 1

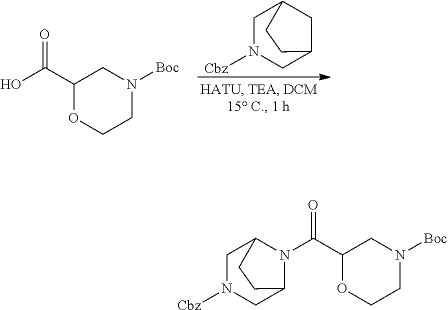 [SMILES: CC(C)(C)OC(=O)N1CCOC(C(=O)O)C1 ; O=C(OCc1ccccc1)N1CC2CCC(C2)C1 ; CC(C)(C)OC(=O)N1CCOC(C(=O)N2C3CCC2CN(C(=O)OCc2ccccc2)C3)C1]

To a solution of 4-tert-butoxycarbonylmorpholine-2-carboxylic acid (4 g, 17.30 mmol, 1 eq) and benzyl 3,8-diazabicyclo[3.2.1]octane-3-carboxylate (5.11 g, 20.76 mmol, 1.2 eq) in dichloromethane (80 mL) was added triethylamine (8.75 g, 86.49 mmol, 12 mL, 5 eq) and O-(7-azabenzotriazol-1-yl)-N,N,N′,N′-tetramethyluronium hexafluorophosphate (9.87 g, 25.95 mmol, 1.5 eq). The mixture was stirred at 15° C. for 1 hours. Water (80 mL) was added, and the mixture was extracted with dichloromethane (100 mL) three times, and then the combined organic layers were dried over anhydrous sodium sulfate and concentrated. The residue was purified by semi-preparative reverse phase chromatography (column: Phenomenex luna c18 250 mm*100 mm*10 um; mobile phase: [water (0.225% FA)-ACN]; B %: 35%-65%, 22 min). Compound tert-butyl 2-(3-benzyloxycarbonyl-3,8-diazabicyclo[3.2.1]octane-8-carbonyl) morpholine-4-carboxylate (7.9 g, 17.19 mmol) was obtained as a white solid.

### Step 2

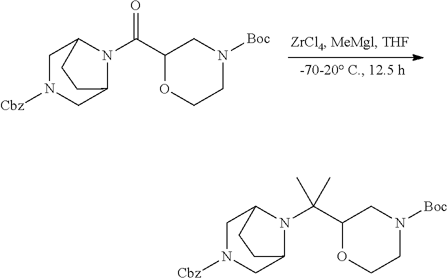 [SMILES: CC(C)(C)OC(=O)N1CCOC(C(=O)N2C3CCC2CN(C(=O)OCc2ccccc2)C3)C1 ; CC(C)(C)OC(=O)N1CCOC(C(C)(C)N2C3CCC2CN(C(=O)OCc2ccccc2)C3)C1]

To a solution of zirconium(iv) chloride (6.41 g, 27.51 mmol, 2.3 mL, 1.6 eq) in tetrahydrofuran (280 mL) at −70° C. was added tert-butyl 2-(3-benzyloxycarbonyl-3,8-diazabicyclo[3.2.1]octane-8-carbonyl) morpholine-4-carboxylate (7.9 g, 17.19 mmol, 1 eq) in tetrahydrofuran (40 mL), and then methylmagnesium bromide (3 M, 46 mL, 8 eq) was added. The mixture was stirred at −70° C. for 0.5 hours, then the resulting mixture was warmed to 20° C. and stirred for 12 hours. The mixture was poured into saturated ammonium chloride solution (400 mL) and extracted with ethyl acetate (400 mL) three times. The combined organic layers was washed by brine (600 mL) and dried over anhydrous sodium sulfate, concentrated to afford the crude product. The residue was purified by semi-preparative reverse phase chromatography (column: Kromasil Eternity XT 250*80 mm*10 um; mobile phase: [water (0.05% ammonia hydroxide v/v)-ACN]; B %: 60%-90%, 27 min); and then was purified by prep-HPLC (column: Phenomenex luna C18(250*70 mm, 15 um); mobile phase: [water (0.225% FA)-ACN]; B %: 30ACN %-60ACN %, 15 min) to afford tert-butyl 2-[1-(3-benzyloxycarbonyl-3,8-diazabicyclo [3.2.1]octan-8-yl)-1-methyl-ethyl]morpholine-4-carboxylate (827 mg, 1.75 mmol)

tert-Butyl 2-[1-(3-benzyloxycarbonyl-3,8-diazabicyclo [3.2.1]octan-8-yl)-1-methyl-ethyl]morpholine-4-carboxylate was converted to the title compound according to the schemes below using procedures described or referenced above, as well as general procedures known to those skilled in the art.

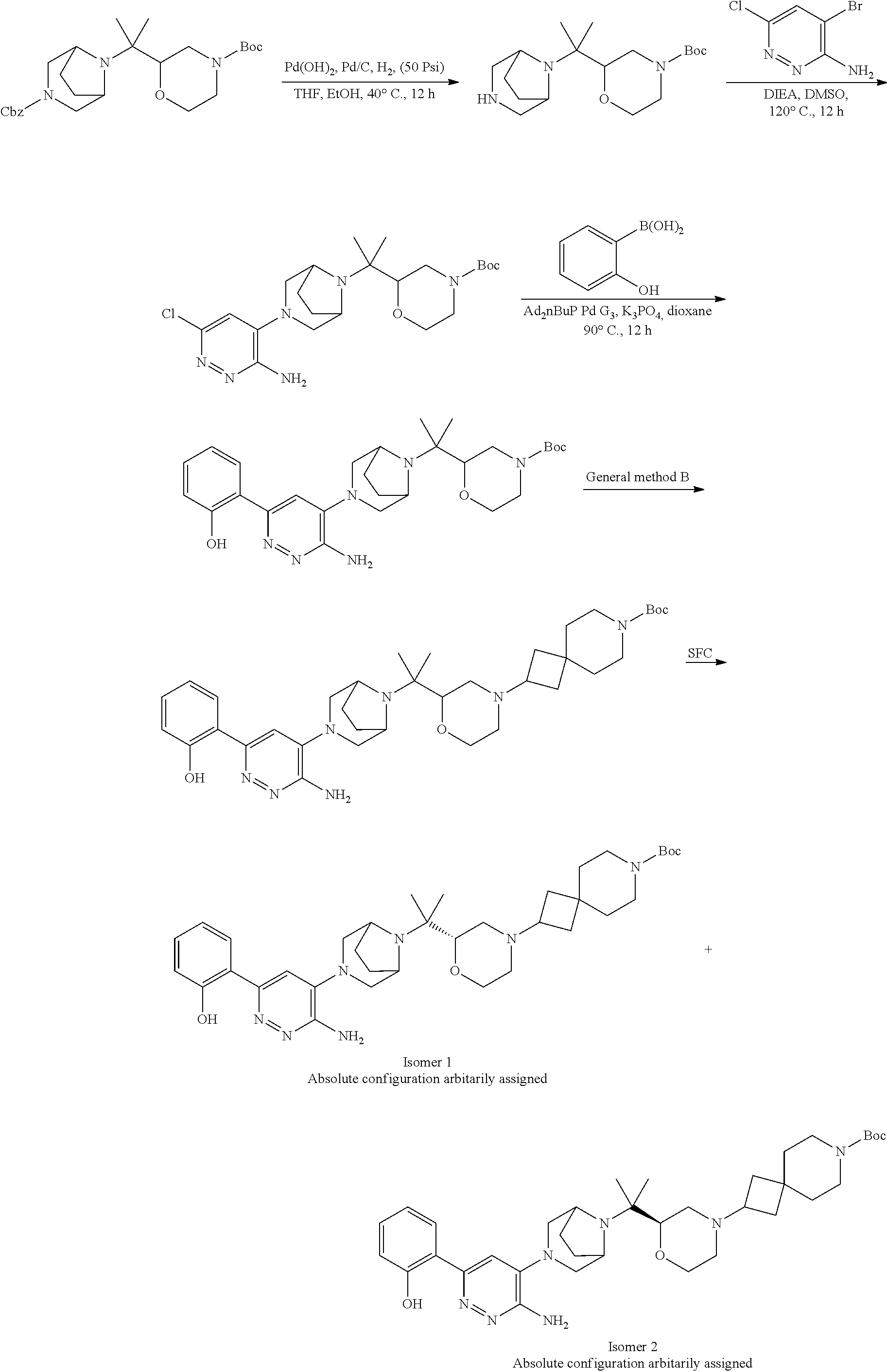

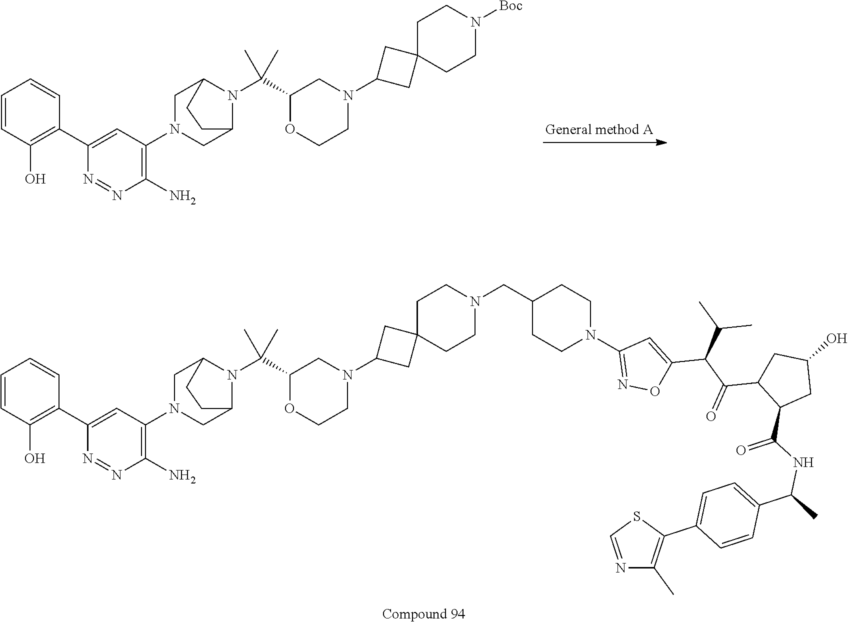 [SMILES: CC(C)(C)OC(=O)N1CCC2(CC1)CC(N1CCO[C@H](C(C)(C)N3C4CCC3CN(c3cc(-c5ccccc5O)nnc3N)C4)C1)C2 ; Cc1ncsc1-c1ccc([C@H](C)NC(=O)[C@@H]2C[C@@H](O)CC2C(=O)[C@@H](c2cc(N3CCC(CN4CCC5(CC4)CC(N4CCO[C@H](C(C)(C)N6C7CCC6CN(c6cc(-c8ccccc8O)nnc6N)C7)C4)C5)CC3)no2)C(C)C)cc1]

Compound 95 was prepared using analogous procedures.

### Exemplary Synthesis of Compound 96

Prepared according to the schemes below using procedures described above, as well as general procedures known to those skilled in the art.

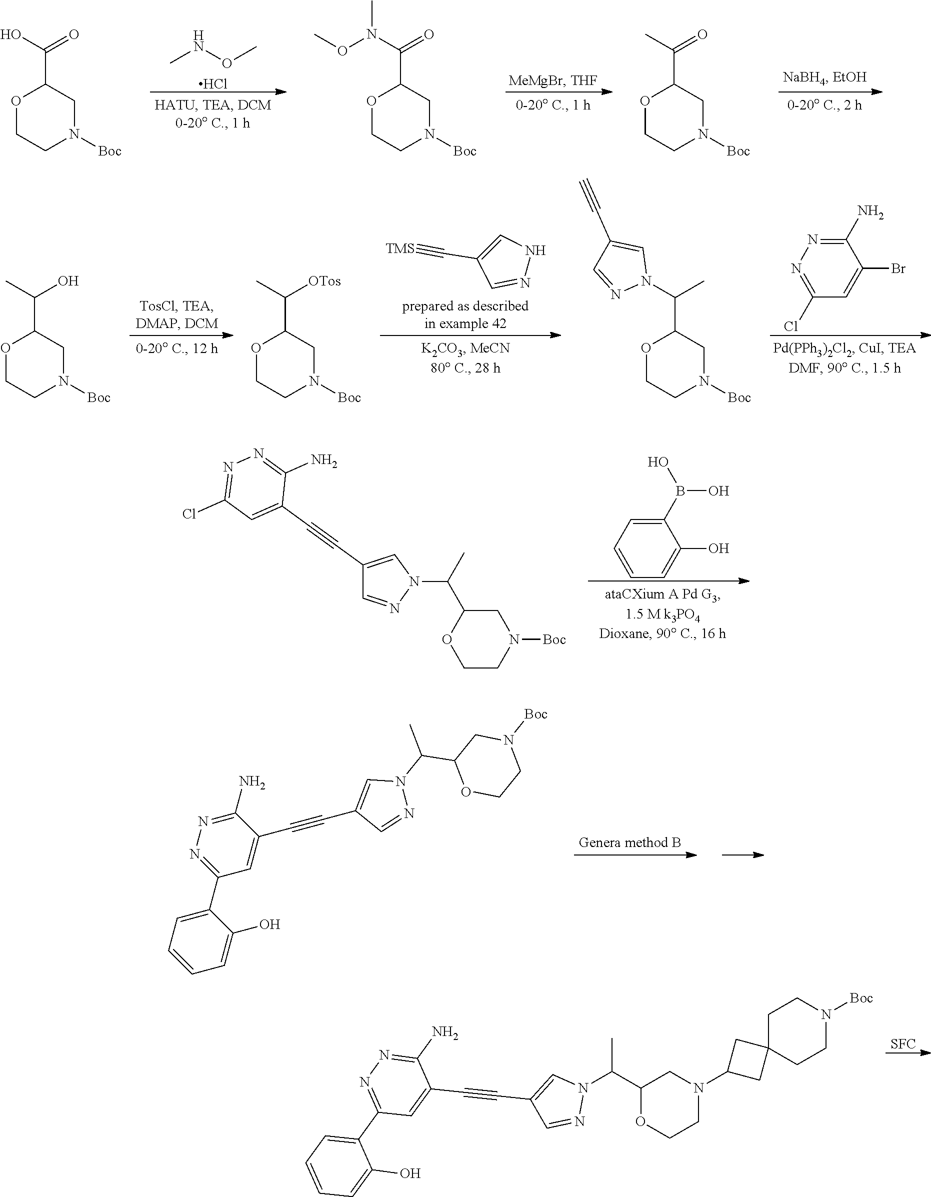

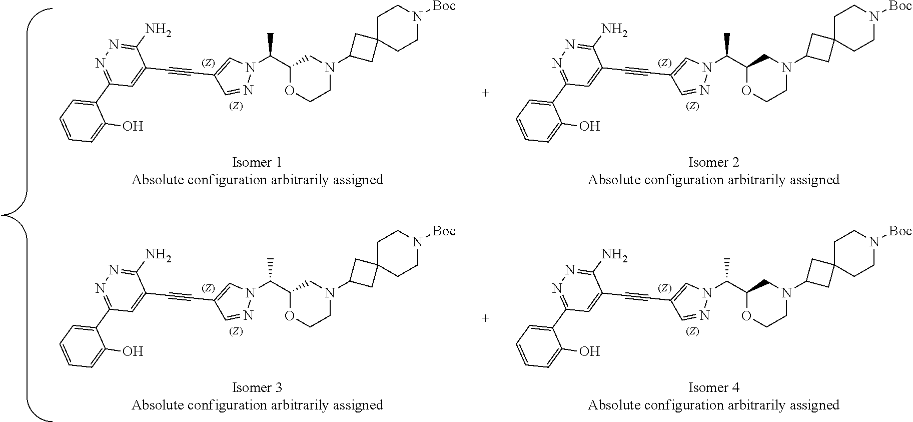 [SMILES: C[C@@H]([C@@H]1CN(C2CC3(CCN(C(=O)OC(C)(C)C)CC3)C2)CCO1)n1cc(C#Cc2cc(-c3ccccc3O)nnc2N)cn1 ; C[C@@H]([C@H]1CN(C2CC3(CCN(C(=O)OC(C)(C)C)CC3)C2)CCO1)n1cc(C#Cc2cc(-c3ccccc3O)nnc2N)cn1 ; C[C@H]([C@@H]1CN(C2CC3(CCN(C(=O)OC(C)(C)C)CC3)C2)CCO1)n1cc(C#Cc2cc(-c3ccccc3O)nnc2N)cn1 ; C[C@H]([C@H]1CN(C2CC3(CCN(C(=O)OC(C)(C)C)CC3)C2)CCO1)n1cc(C#Cc2cc(-c3ccccc3O)nnc2N)cn1]

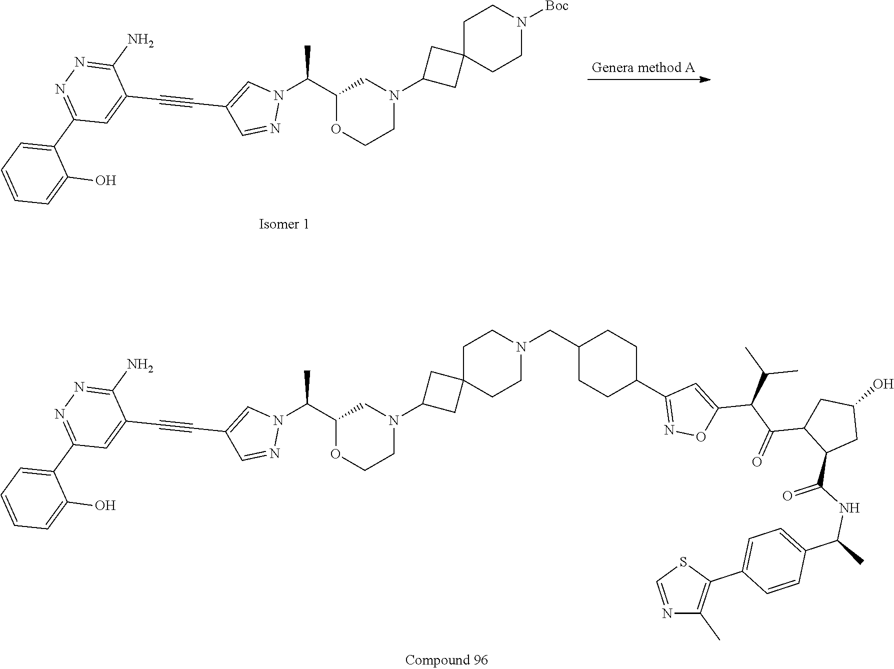 [SMILES: C[C@@H]([C@@H]1CN(C2CC3(CCN(C(=O)OC(C)(C)C)CC3)C2)CCO1)n1cc(C#Cc2cc(-c3ccccc3O)nnc2N)cn1 ; Cc1ncsc1-c1ccc([C@H](C)NC(=O)[C@@H]2C[C@@H](O)CC2C(=O)[C@@H](c2cc(C3CCC(CN4CCC5(CC4)CC(N4CCO[C@H]([C@H](C)n6cc(C#Cc7cc(-c8ccccc8O)nnc7N)cn6)C4)C5)CC3)no2)C(C)C)cc1]

Compounds 98, 98, and 99 were prepared using analogous procedures.

### Exemplary Synthesis of Compound 101

Compound 101 was prepared according to the scheme below using procedures described above.

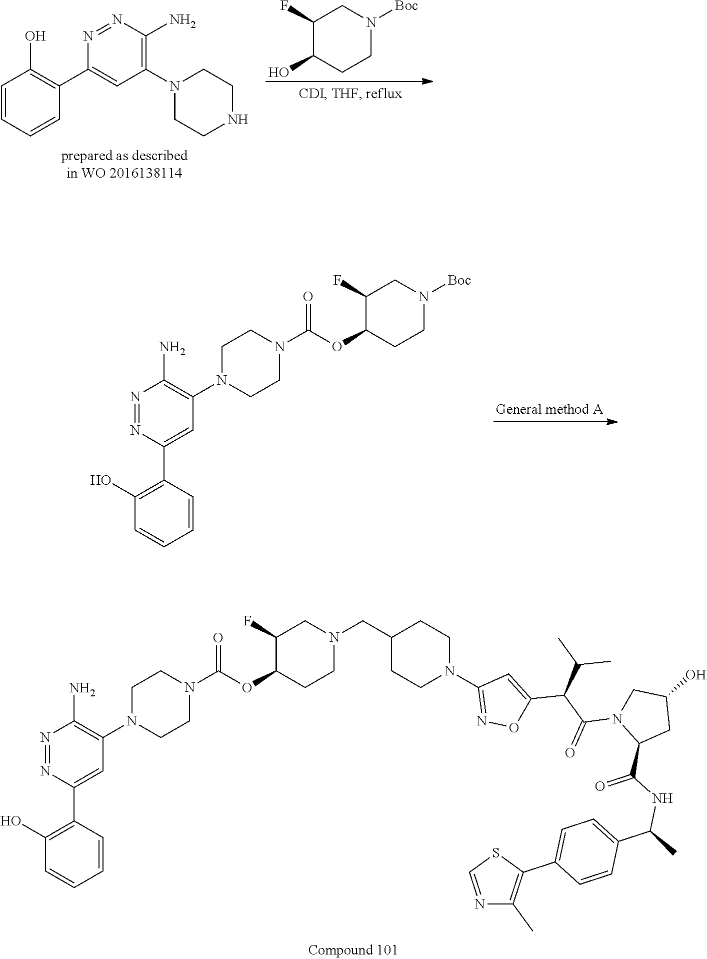 [SMILES: Nc1nnc(-c2ccccc2O)cc1N1CCNCC1 ; CC(C)(C)OC(=O)N1CC[C@@H](O)[C@@H](F)C1 ; CC(C)(C)OC(=O)N1CC[C@@H](OC(=O)N2CCN(c3cc(-c4ccccc4O)nnc3N)CC2)[C@@H](F)C1 ; Cc1ncsc1-c1ccc([C@H](C)NC(=O)[C@@H]2C[C@@H](O)CN2C(=O)[C@@H](c2cc(N3CCC(CN4CC[C@@H](OC(=O)N5CCN(c6cc(-c7ccccc7O)nnc6N)CC5)[C@@H](F)C4)CC3)no2)C(C)C)cc1]

Compounds 102, 103, and 104 were prepared using analogous procedures.

### Exemplary Synthesis of Compound 105

Prepared according to the schemes below using procedures described or referenced above, as well as general procedures known to those skilled in the art.

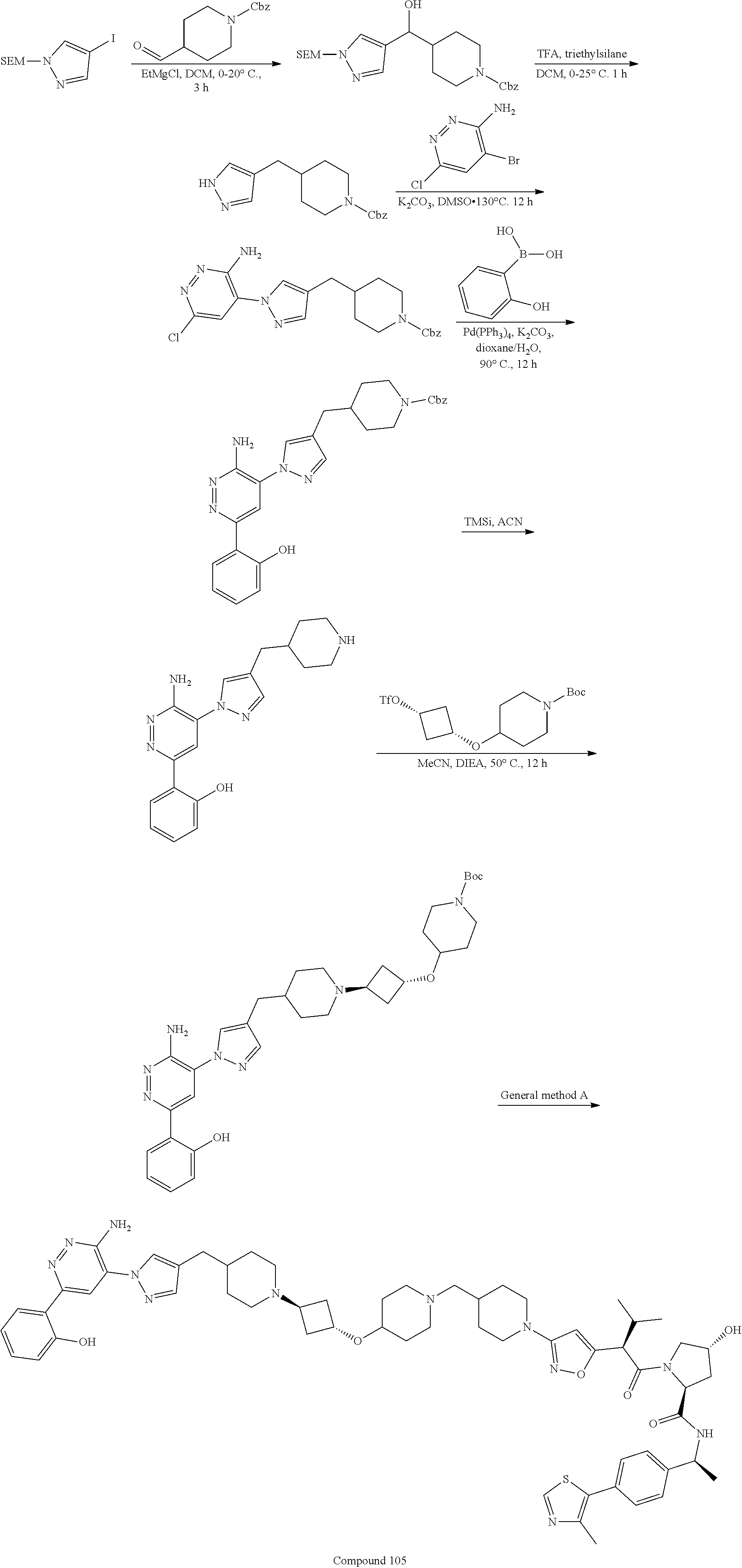

### Exemplary Synthesis of Compound 107

Prepared according to the schemes below using procedures described for other examples above as well as procedures commonly known to those skilled in the art.

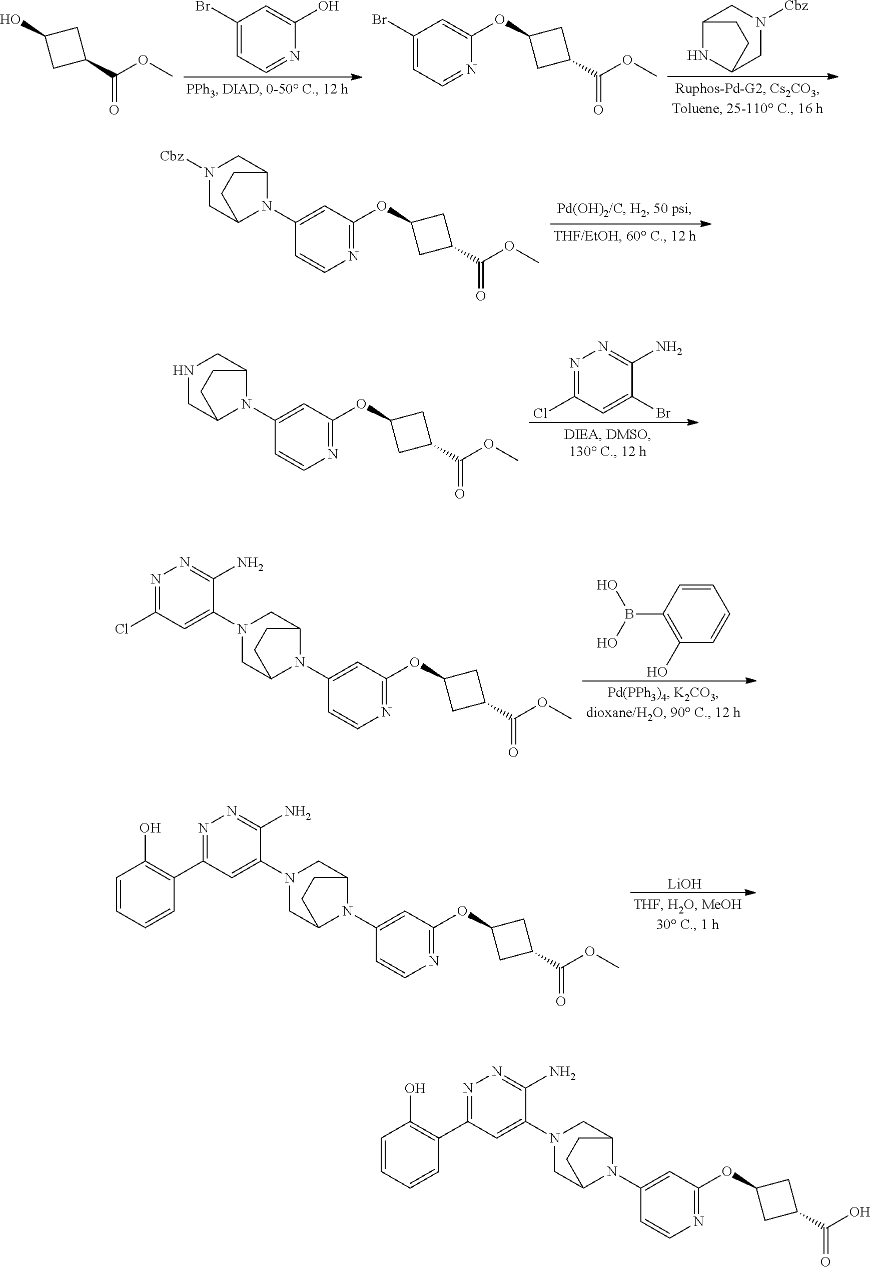

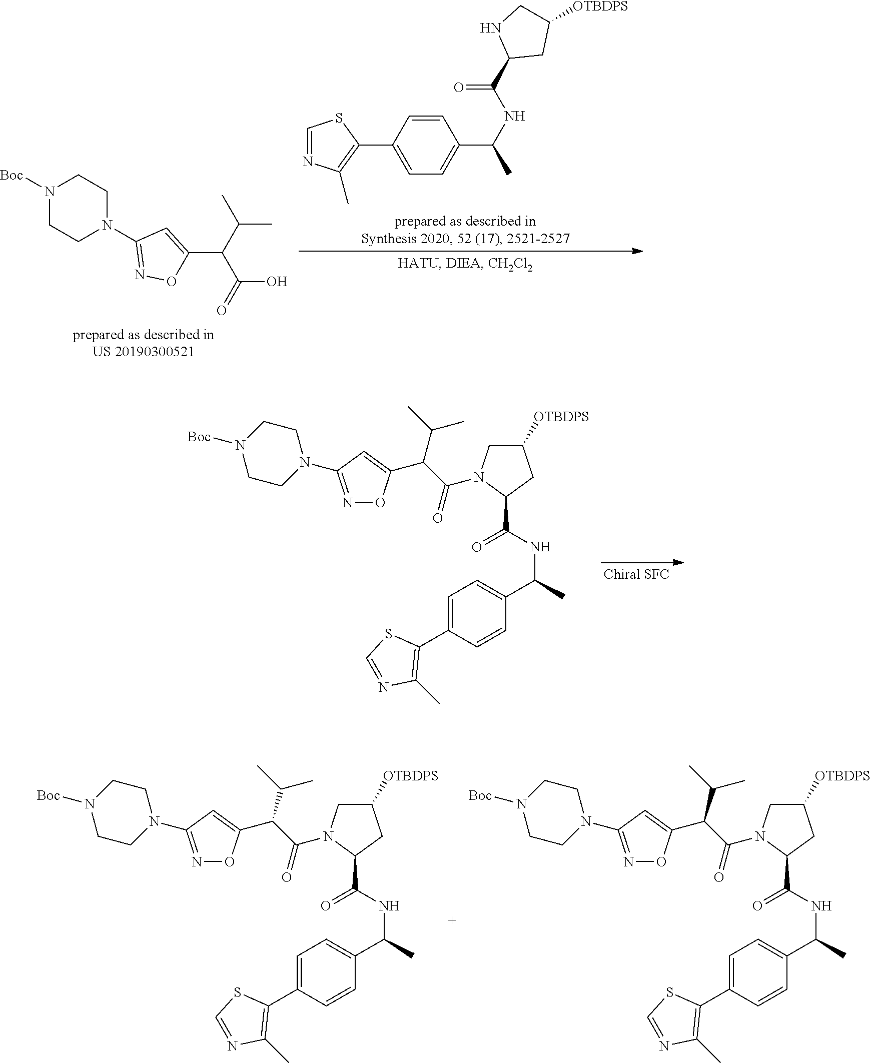

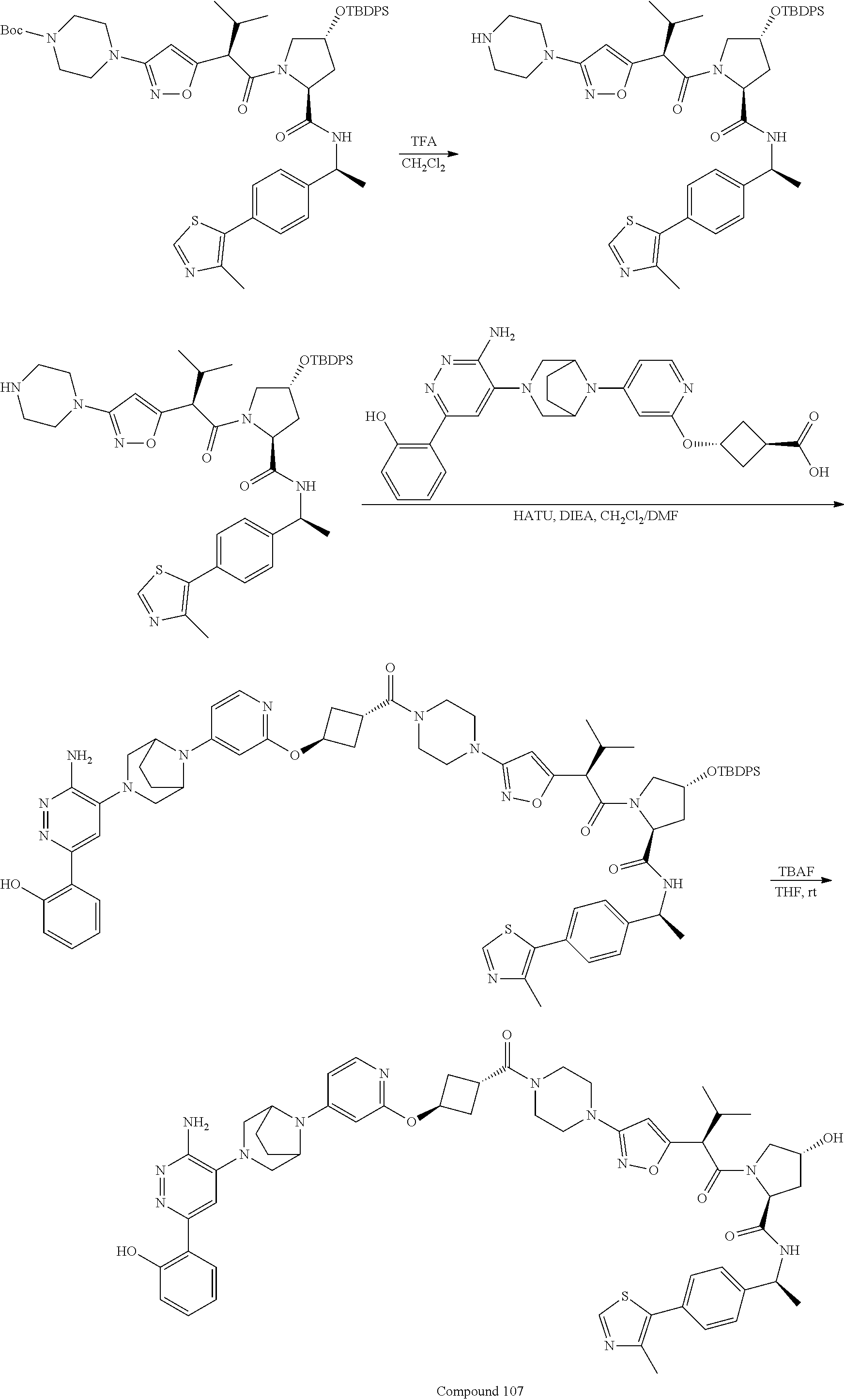

Compound 118 was prepared using analogous procedures.

### Exemplary Synthesis of Compound 108

Compound 108 was prepared according to the schemes below using procedures described for other examples above as well as procedures commonly known to those skilled in the art.

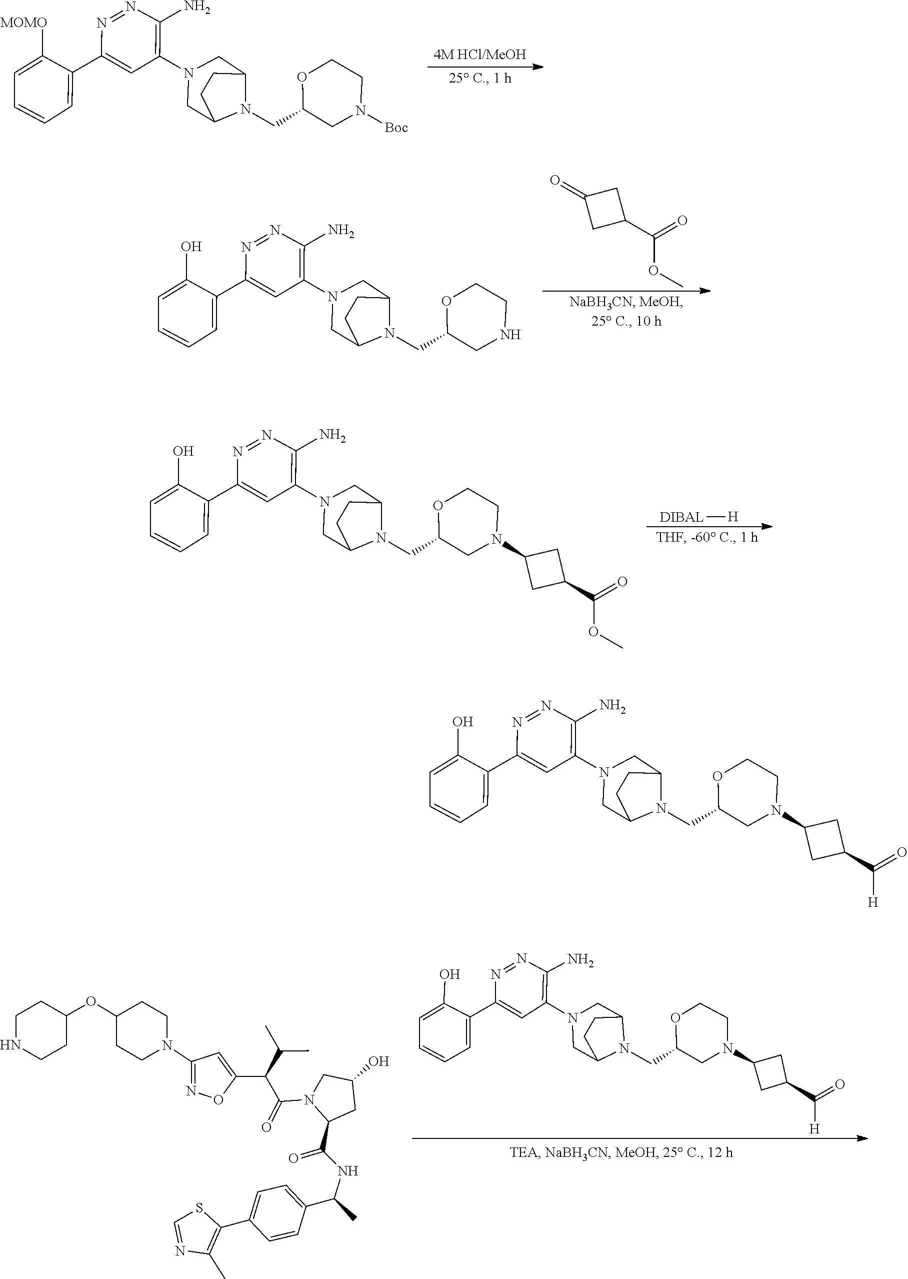

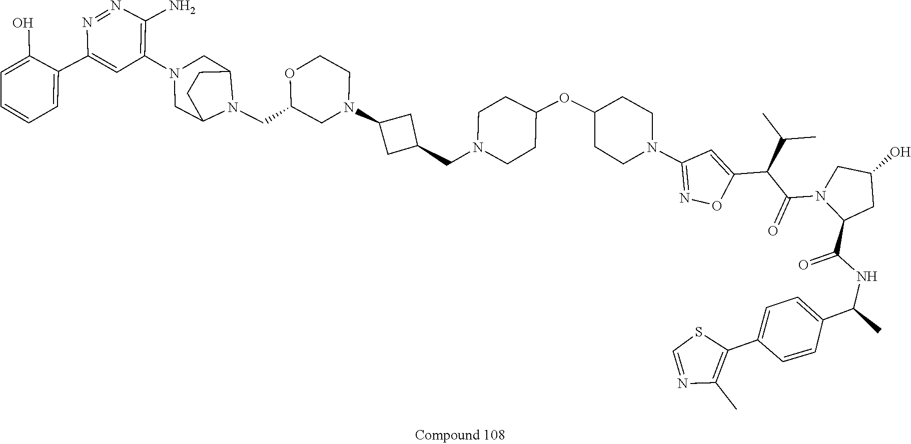 [SMILES: Cc1ncsc1-c1ccc([C@H](C)NC(=O)[C@@H]2C[C@@H](O)CN2C(=O)[C@@H](c2cc(N3CCC(OC4CCN(C[C@H]5C[C@@H](N6CCO[C@@H](CN7C8CCC7CN(c7cc(-c9ccccc9O)nnc7N)C8)C6)C5)CC4)CC3)no2)C(C)C)cc1]

### Exemplary Synthesis of Compound 109

Prepared according to the schemes below using procedures described for other examples above as well as procedures commonly known to those skilled in the art.

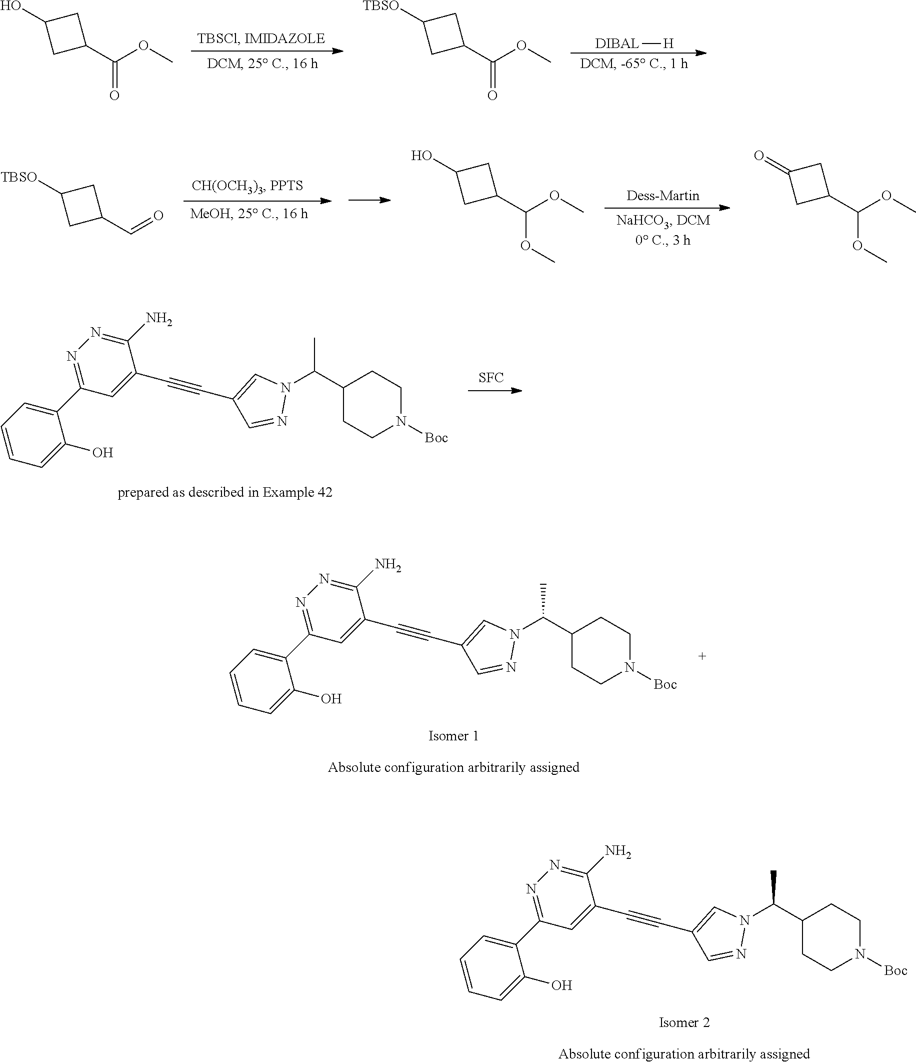

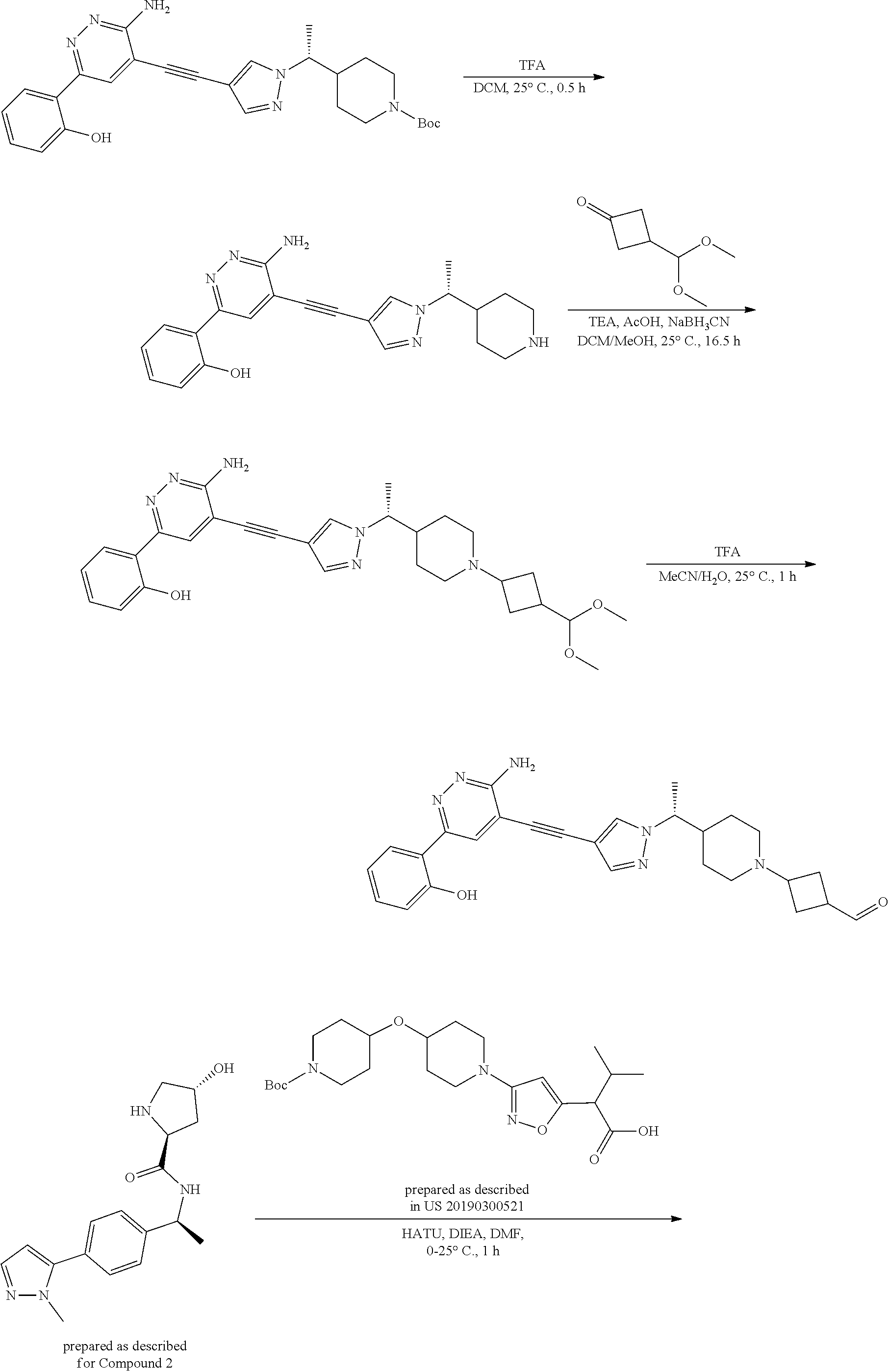

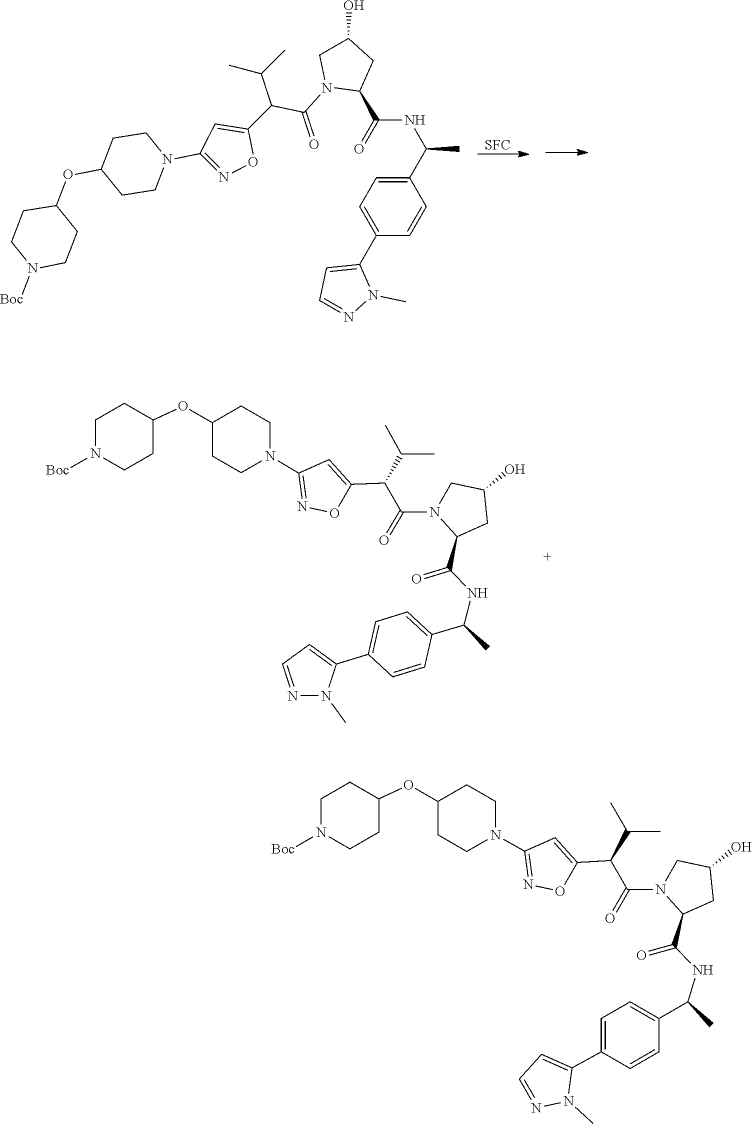

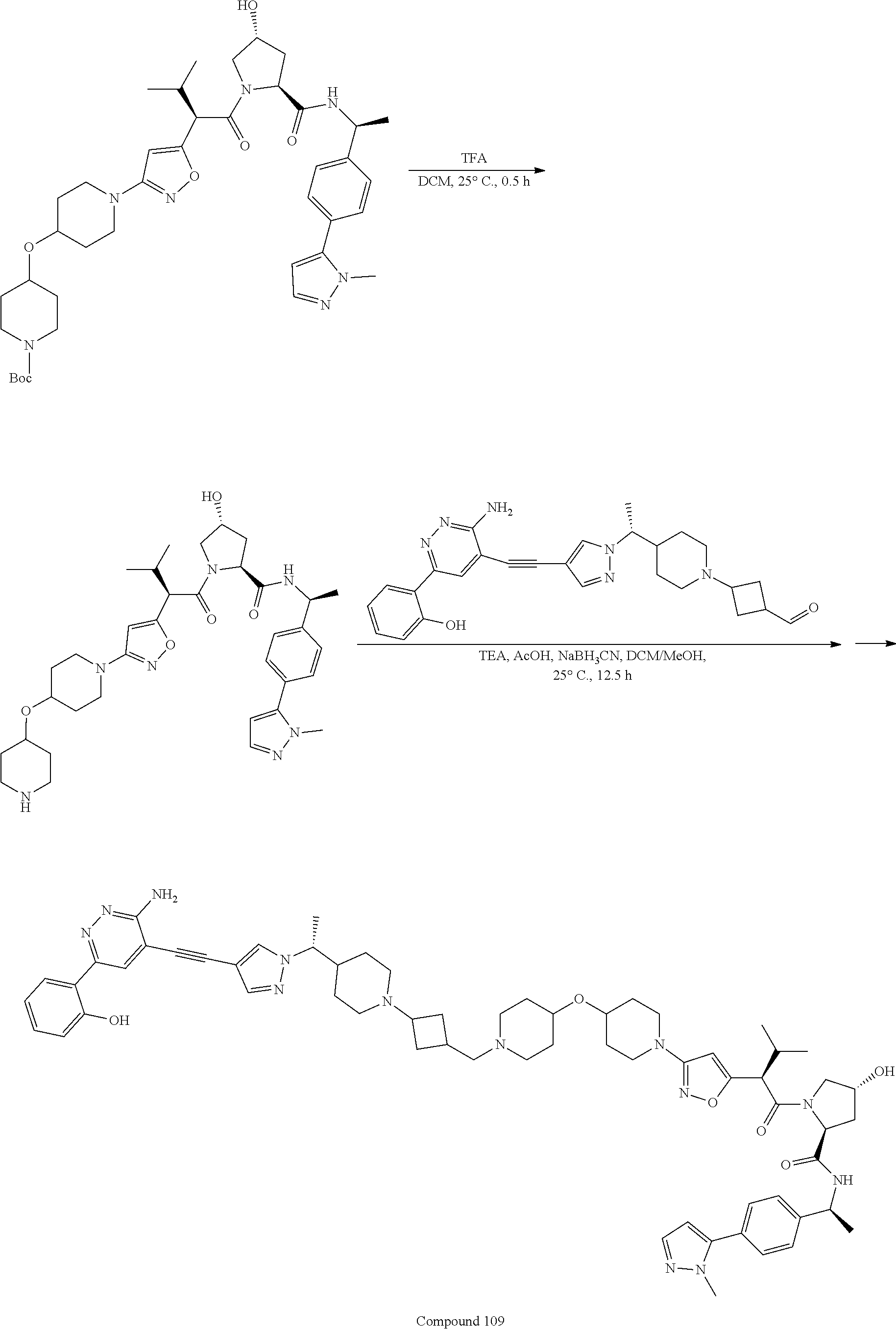

Compounds 110, 111, and 112 were prepared using analogous procedures.

### Exemplary Synthesis of Compound 117

Prepared according to the schemes below using procedures described or referenced above, as well as general procedures known to those skilled in the art.

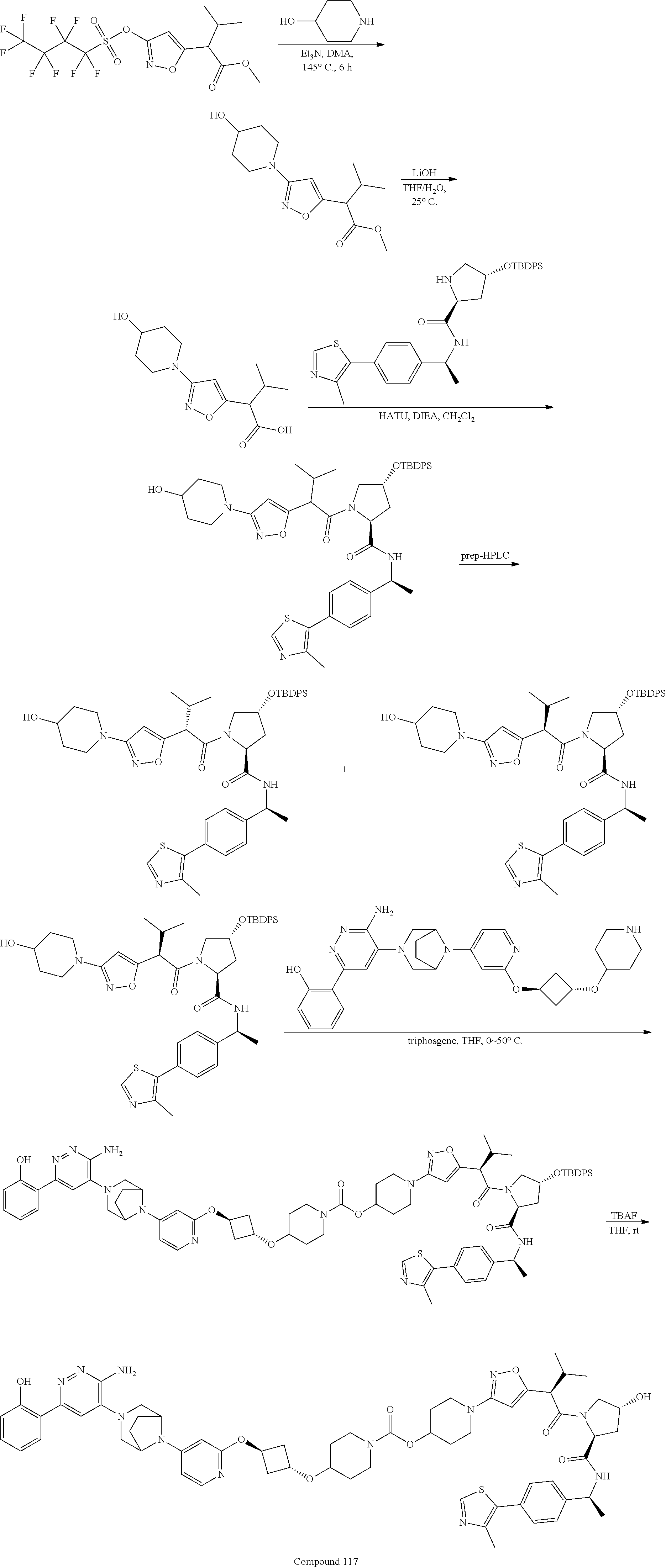

Using analogous procedures, or procedures analogous to that of Compound 41, the following compounds were prepared: 116, 145, 128, 131, 141, and 145.

### Exemplary Synthesis of Compound 119

Prepared according to the schemes below using procedures described above for Compound 35, as well as general procedures known to those skilled in the art.

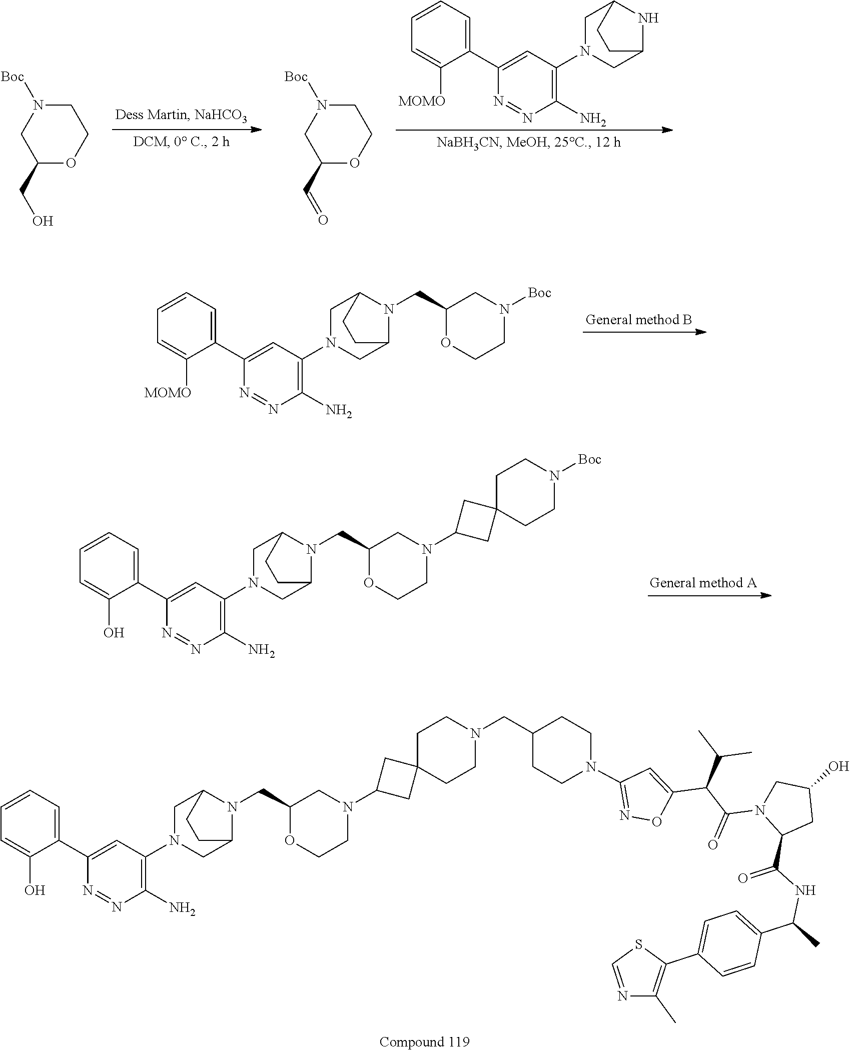

### Exemplary Synthesis of Compound 120

Prepared according to the schemes below using procedures described above, as well as general procedures known to those skilled in the art.

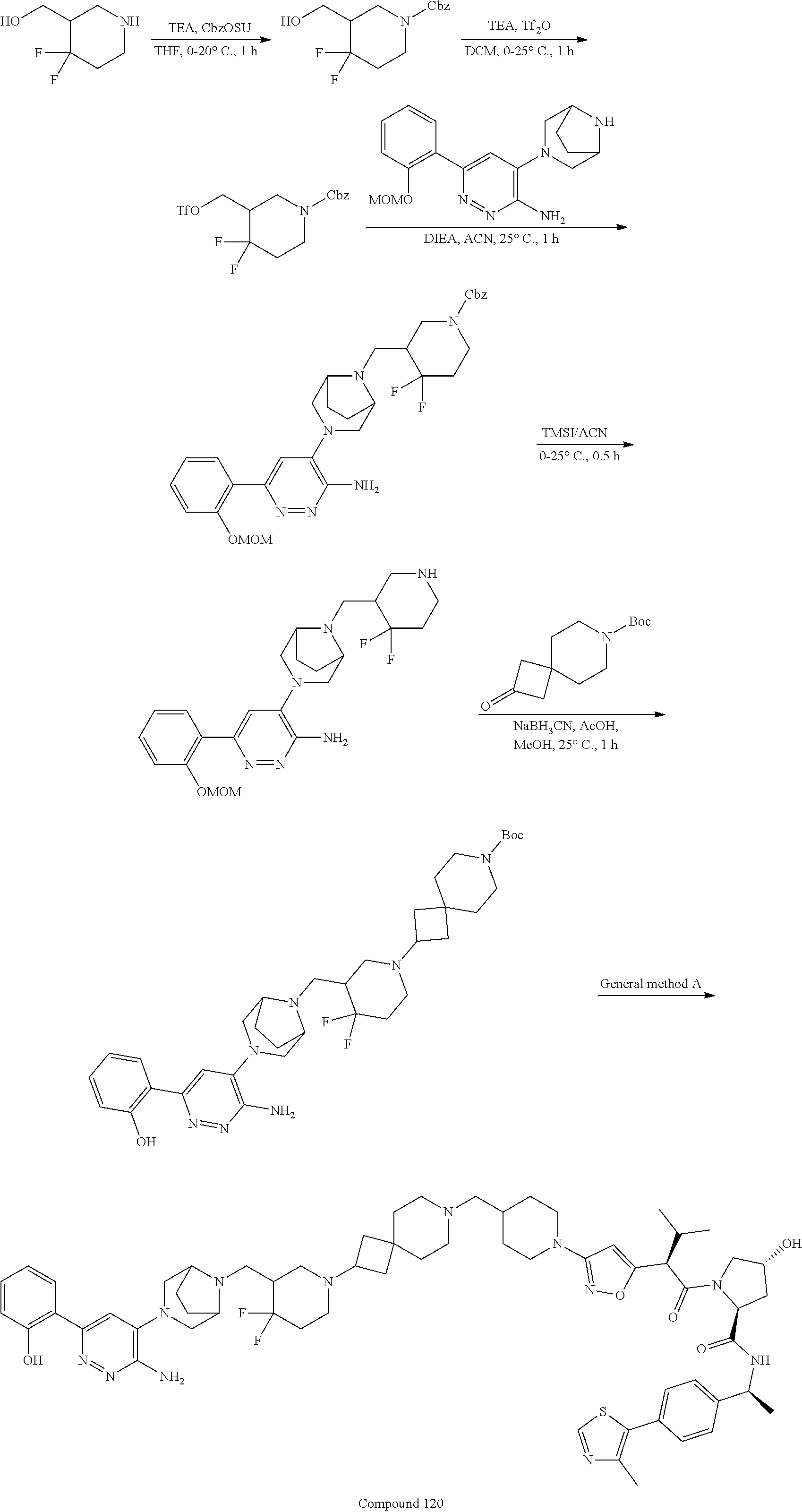

### Exemplary Synthesis of Compound 121

Prepared according to the schemes below using procedures described above, as well as general procedures known to those skilled in the art.

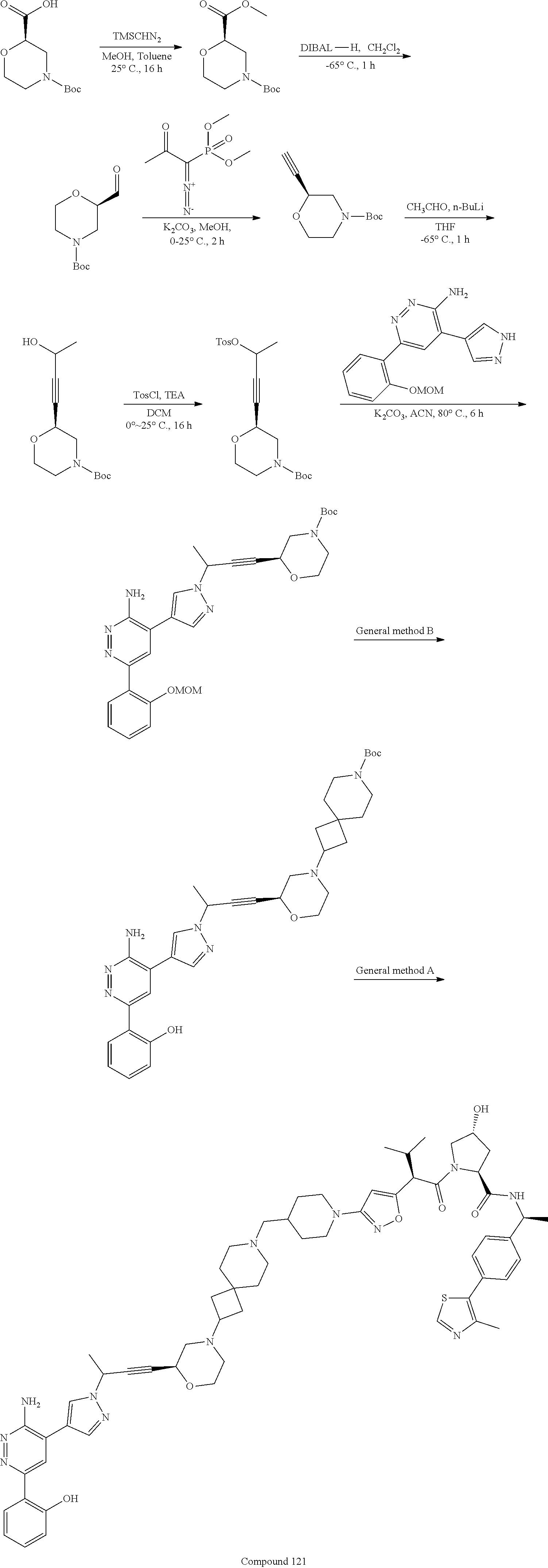

### Exemplary Synthesis of Compound 122

Prepared according to the schemes below using procedures described above, as well as general procedures known to those skilled in the art.

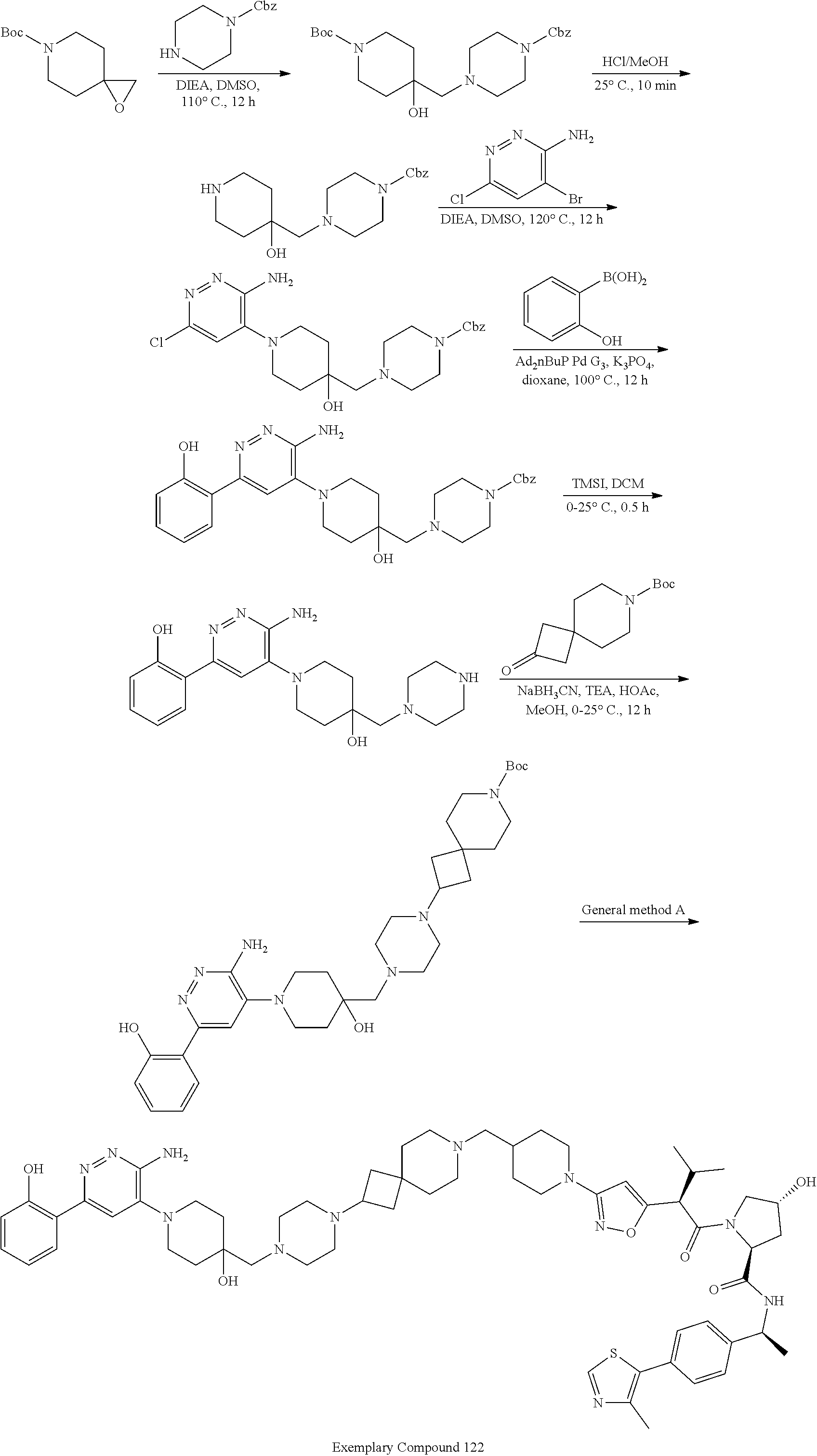

### Exemplary Synthesis of Compound 123

Prepared according to the schemes below using procedures described above, as well as general procedures known to those skilled in the art.

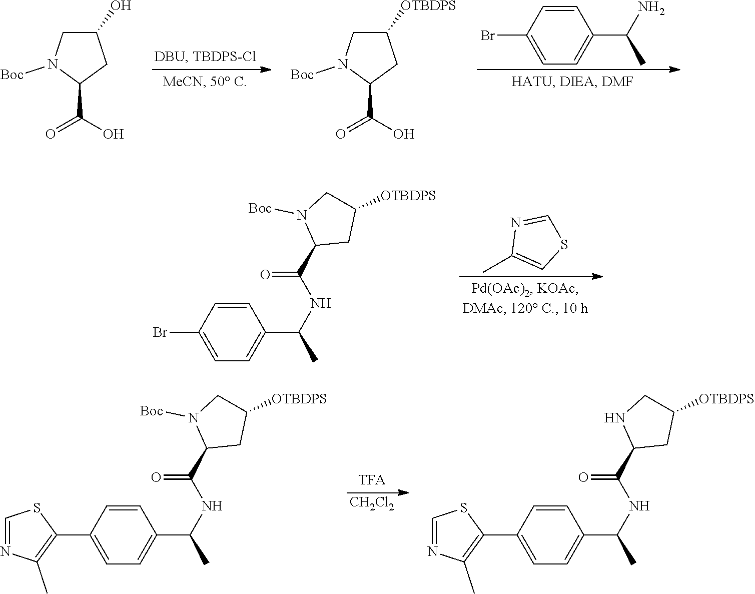

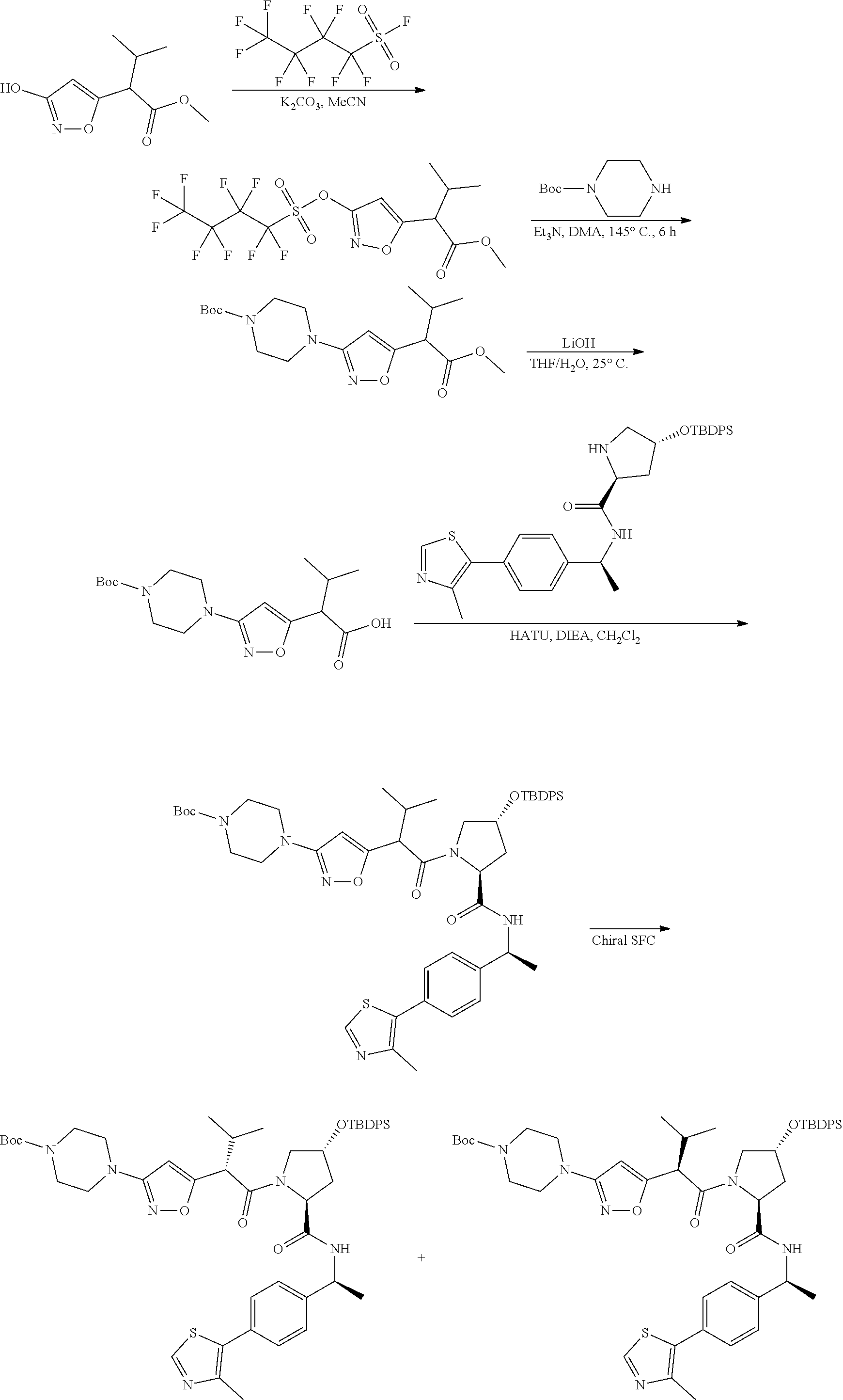

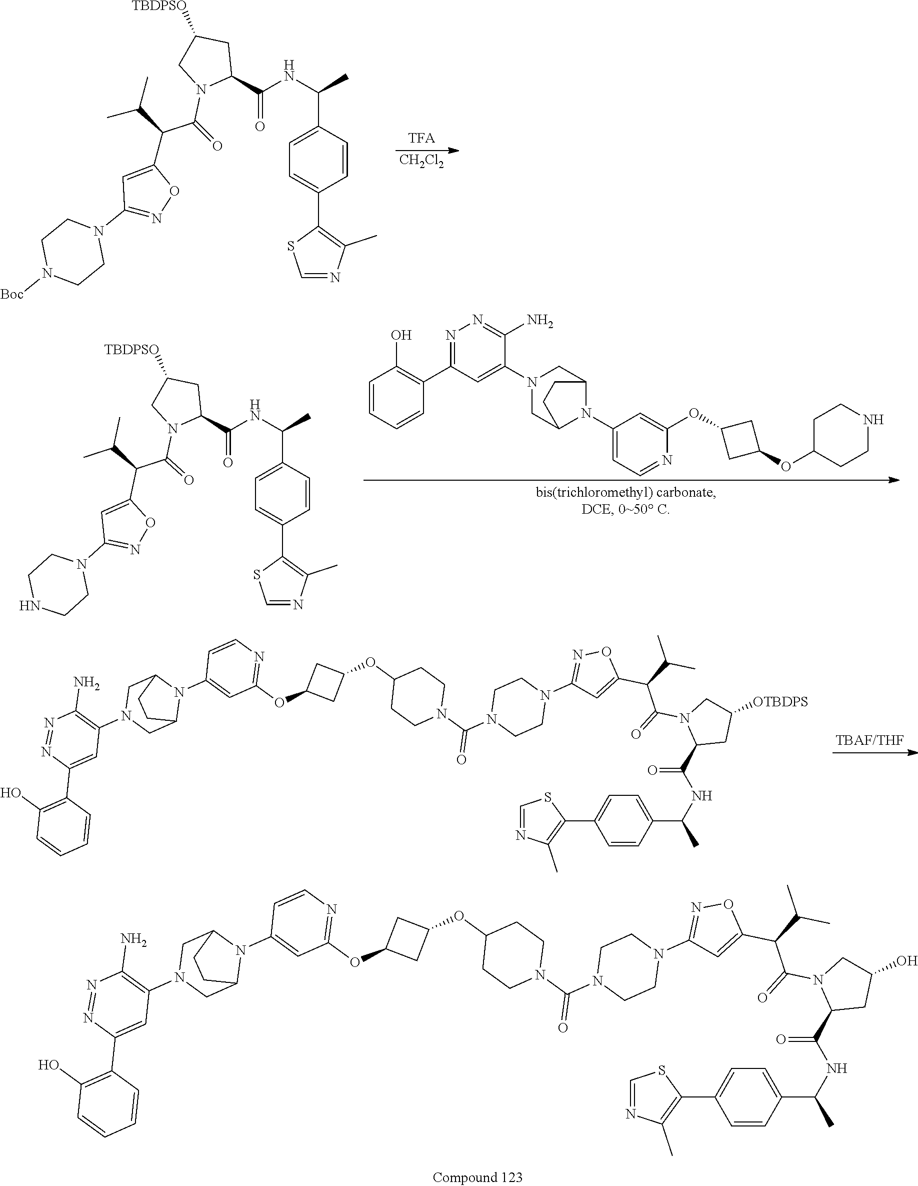

Compound 124 was prepared using analogous procedures.

### Exemplary Synthesis of Compound 125

Compound 125 prepared according to the schemes below using procedures described above, as well as general procedures known to those skilled in the art.

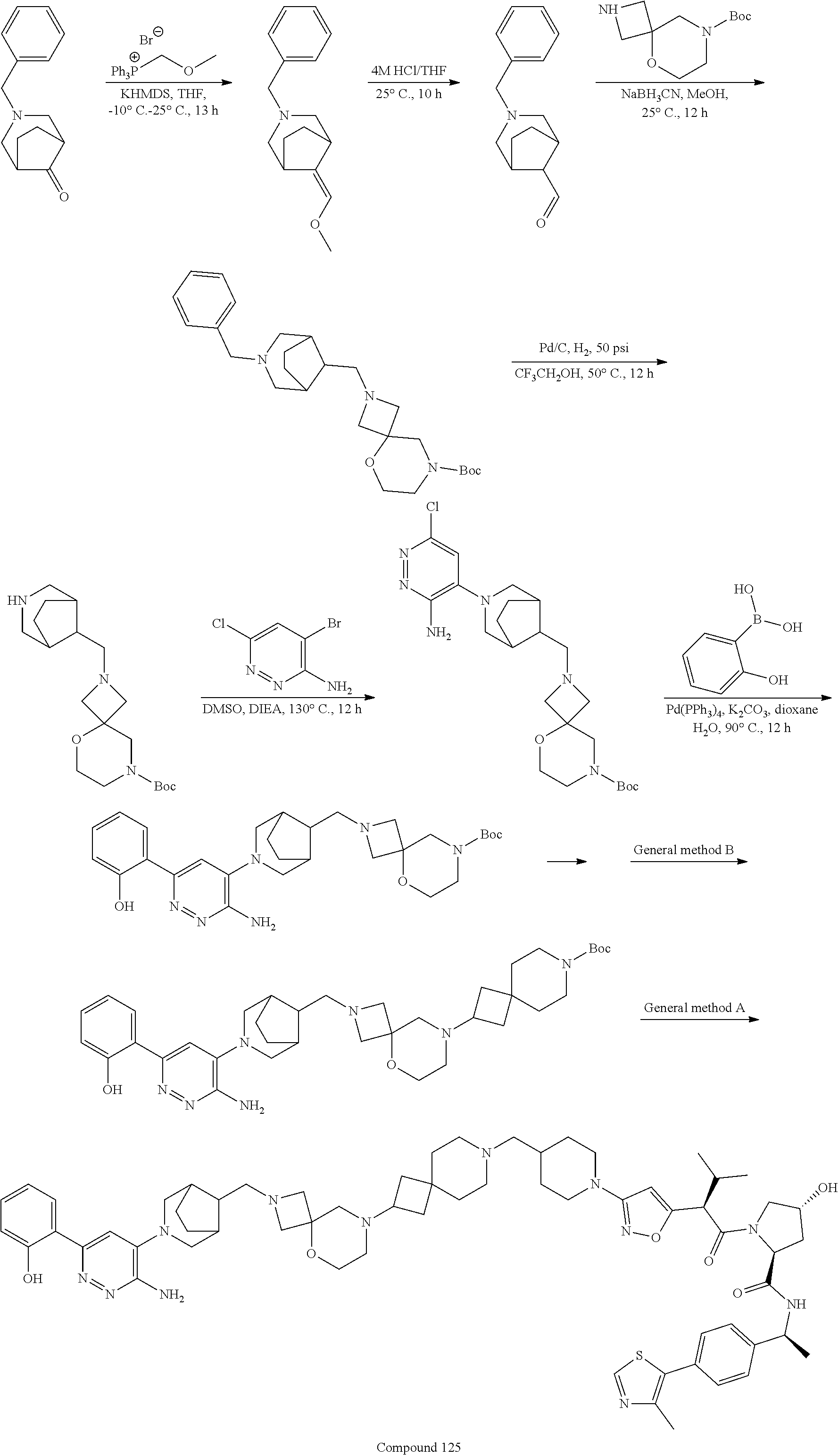

### Exemplary Synthesis of Compound 126

Compound 126 prepared according to the schemes below using procedures described above, as well as general procedures known to those skilled in the art.

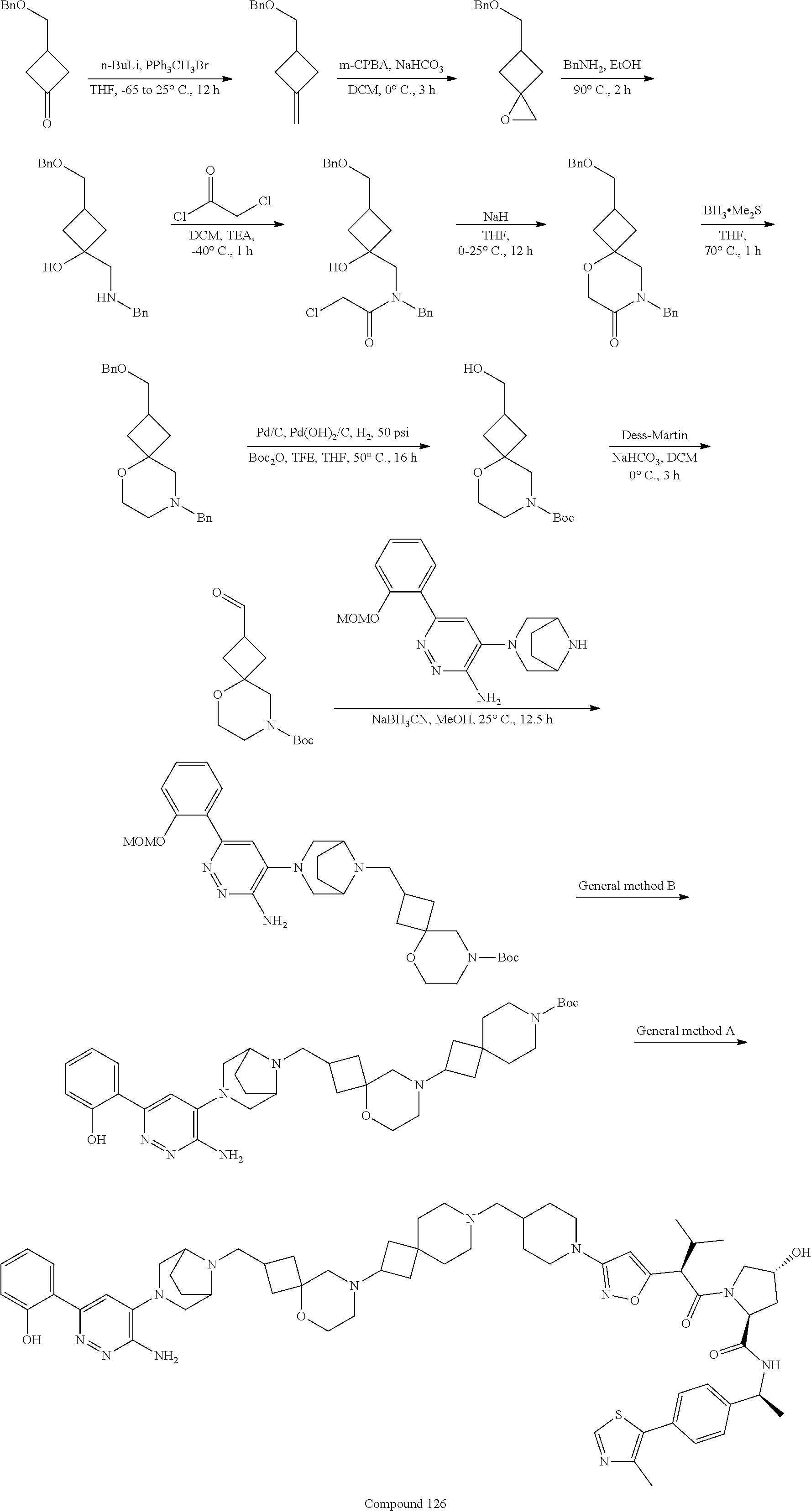

### Exemplary Synthesis of Compound 128

### Step 1

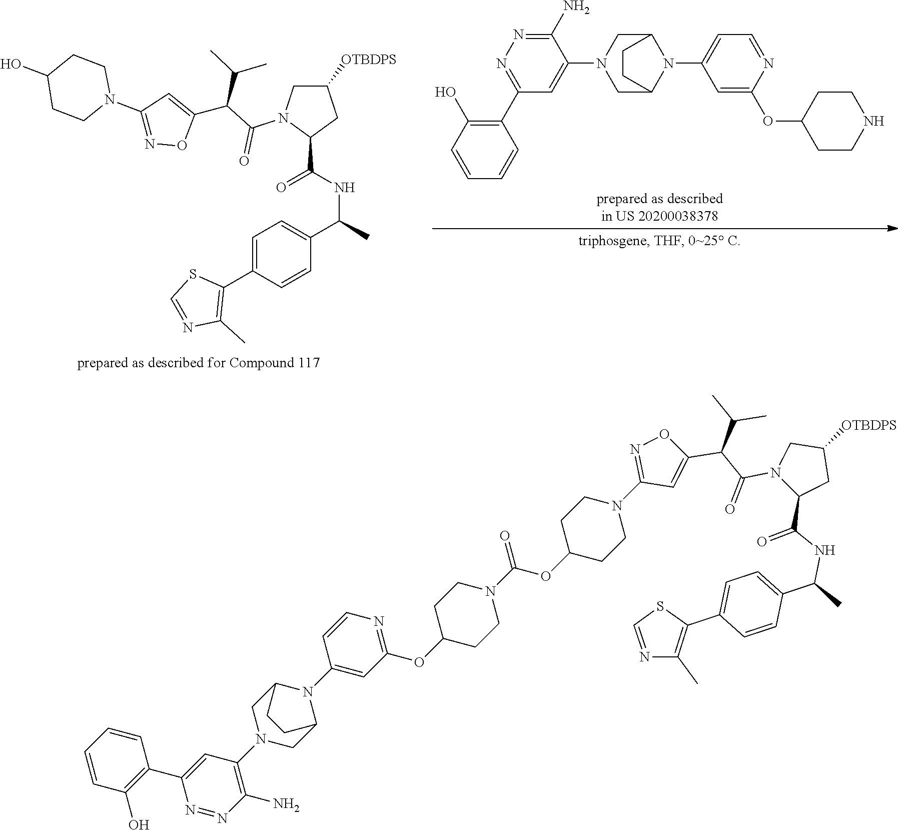

To a solution of bis(trichloromethyl) carbonate (23.16 mg, 78.04 umol, 0.8 eq) in THF (2 mL) was added the solution of 2-[6-amino-5-[8-[2-(4-piperidyloxy)-4-pyridyl]-3,8-diazabicyclo[3.2.1]octan-3-yl]pyridazin-3-yl]phenol (69 mg, 0.146 mmol, 1.5 eq) and TEA (59 mg, 0.585 mmol, 81.46 uL, 6 eq) in THF (2 mL) at 0° C. The mixture was stirred at 0° C. for 1 hour. Then (2S,4R)-4-[tert-butyl(diphenyl)silyl]oxy-1-[(2S)-2-[3-(4-hydroxy-1-piperidyl)isoxazol-5-yl]-3-methyl-butanoyl]-N-[(1S)-1-[4-(4-methylthiazol-5-yl)phenyl]ethyl]pyrrolidine-2-carboxamide (80 mg, 0.098 mmol, 1 eq) was added. The mixture was stirred at 25° C. for 1 hour and concentrated in vacuo. The residue was purified by flash silica gel chromatography (ISCO®; 4 g SepaFlash® Silica Flash Column, Eluent of 0-55% ethyl acetate/petroleum ether gradient @ 30 mL/min). Compound (2S,4R)-4-[tert-butyl(diphenyl)silyl]oxy-1-[(2S)-2-[3-(4-hydroxy-1-piperidyl)isoxazol-5-yl]-3-methyl-butanoyl]-N-[(1S)-1-[4-(4-methylthiazol-5-yl)phenyl]ethyl]pyrrolidine-2-carboxamide (100 mg) was obtained as a white solid.

(2S,4R)-4-[tert-butyl(diphenyl)silyl]oxy-1-[(2S)-2-[3-(4-hydroxy-1-piperidyl)isoxazol-5-yl]-3-methyl-butanoyl]-N-[(1S)-1-[4-(4-methylthiazol-5-yl)phenyl]ethyl]pyrrolidine-2-carboxamide was converted to the title compound as described for Compound 117.

Compound 131 was prepared using analogous procedures.

### Exemplary Synthesis of Compound 129

2-(6-amino-5-(8-(2-(azetidin-3-yloxy)pyridin-4-yl)-3,8-diazabicyclo[3.2.1]octan-3-yl)pyridazin-3-yl)phenol was prepared according the scheme below using procedures described for other Examples above, as well as general procedures known to those skilled in the art.

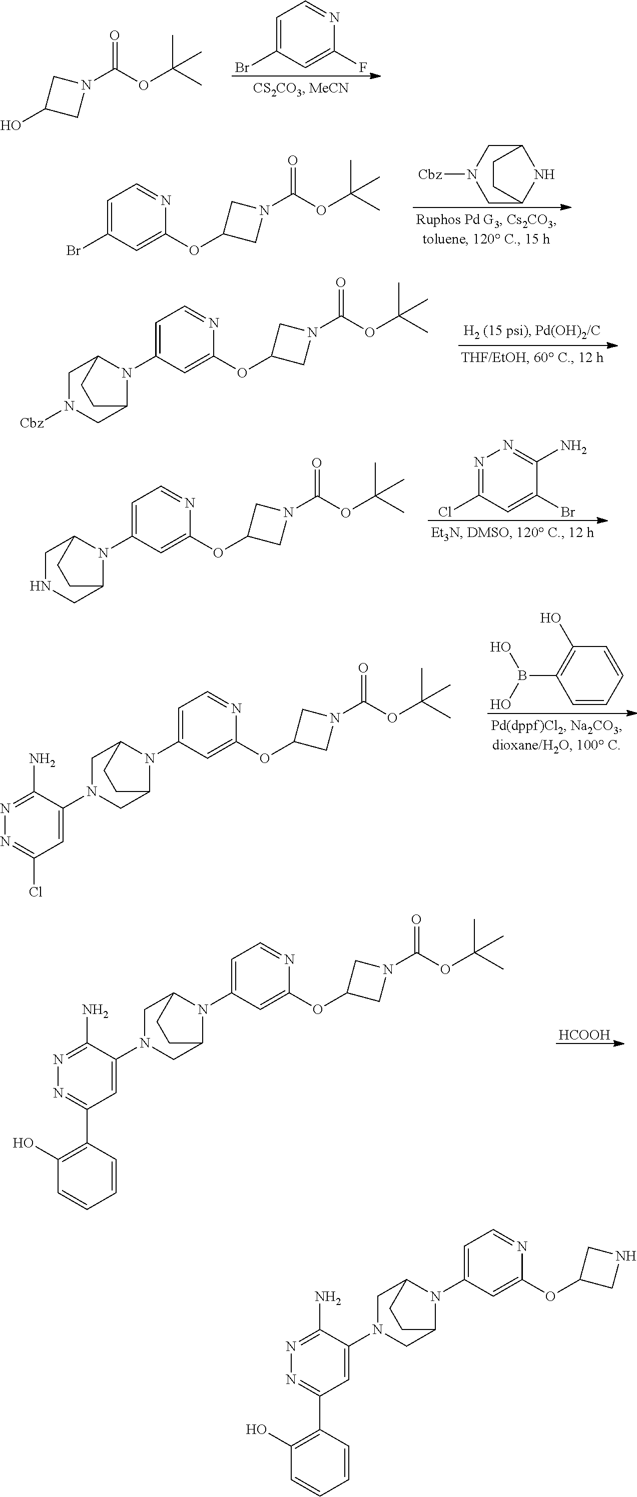

2-(6-amino-5-(8-(2-(azetidin-3-yloxy)pyridin-4-yl)-3,8-diazabicyclo[3.2.1]octan-3-yl)pyridazin-3-yl)phenol was converted to the title compound as described for Compound 123.

### Exemplary Synthesis of Compound 130

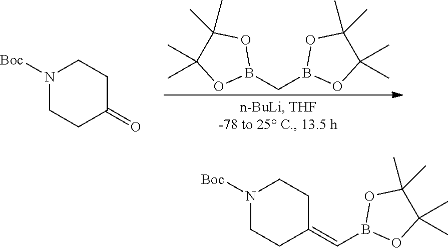

To a solution of 2,2,6,6-tetramethylpiperidine (2.13 g, 15.06 mmol, 2.6 mL, 0.6 eq) in tetrahydrofuran (20 mL) was added dropwise n-butyllithium (2.5 M, 6.0 mL, 0.6 eq) at −30° C., and then the mixture was stirred for 0.5 hours at −30° C. under nitrogen. After that a solution of 4,4,5,5-tetramethyl-2-[(4,4,5,5-tetramethyl-1,3,2-dioxaborolan-2-yl)methyl]-1,3,2-dioxaborolane (3.36 g, 12.55 mmol, 0.5 eq) in tetrahydrofuran (20 mL) was added dropwise at −78° C., and the mixture was stirred for 1 h at −78° C. Then a solution of tert-butyl 4-oxopiperidine-1-carboxylate (5 g, 25.09 mmol, 1 eq) in tetrahydrofuran (40 mL) was added dropwise at −78° C., the mixture was warmed up slowly to 25° C. and stirred for 12 hours at 25° C. The mixture was quenched with saturated ammonium chloride solution (40 mL), and the aqueous phase was extracted with ethyl acetate (40 mL×3). The combined organic phase was dried over anhydrous sodium sulfate, filtered and concentrated in vacuum. It was purified by silica gel column chromatography (petroleum ether/ethyl acetate=from I/O to 100/1). tert-Butyl 4-[(4,4,5,5-tetramethyl-1,3,2-dioxaborolan-2-yl) methylene]piperidine-1-carboxylate (3 g, 9.28 mmol, 37% yield) was obtained as a white solid.

tert-Butyl 4-[(4,4,5,5-tetramethyl-1,3,2-dioxaborolan-2-yl) methylene]piperidine-1-carboxylate was converted to the title compound according to the schemes below using procedures described or referenced above, as well as general procedures known to those skilled in the art.

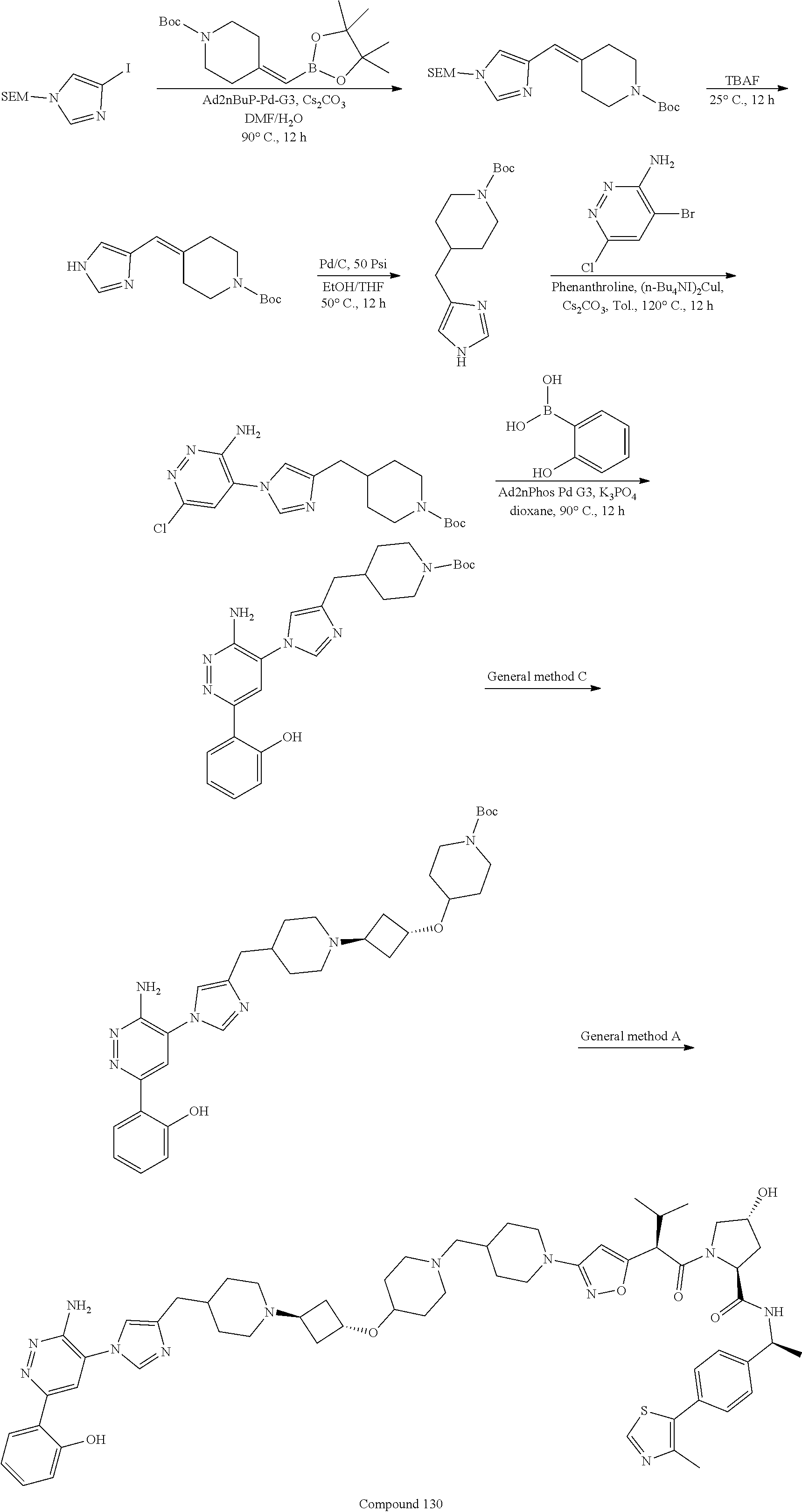

### Exemplary Synthesis of Compound 132

Prepared according to the schemes below using procedures described or referenced above, as well as general procedures known to those skilled in the art.

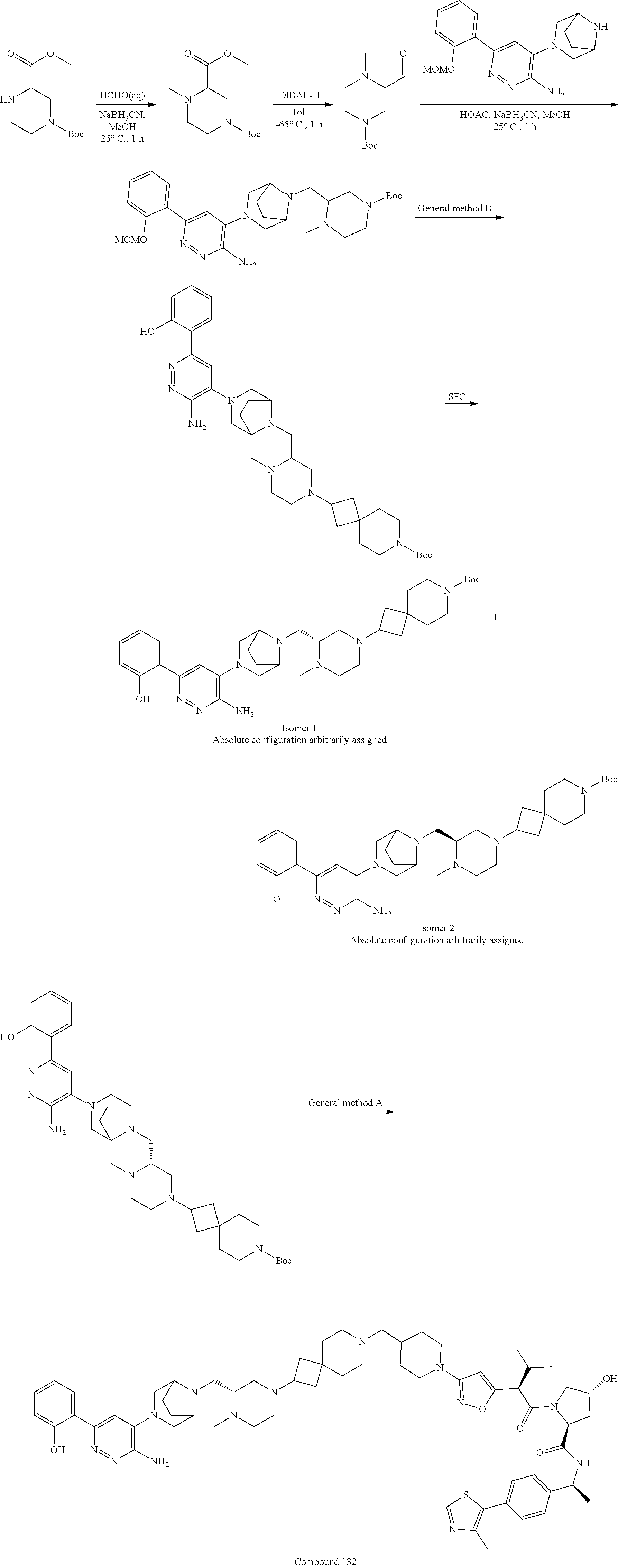

Compound 133 was prepared using analogous procedures.

### Exemplary Synthesis of Compounds 135 and 136

### Step 1

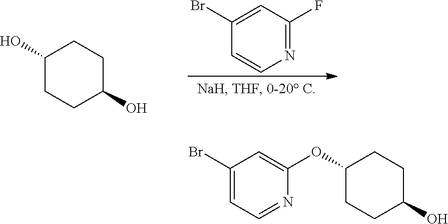 [SMILES: O[C@H]1CC[C@H](O)CC1 ; Fc1cc(Br)ccn1 ; O[C@H]1CC[C@H](Oc2cc(Br)ccn2)CC1]

To a stirred solution of trans-cyclohexane-1,4-diol (3.0 g, 25.8 mmol, 1 eq) and 4-bromo-2-fluoro-pyridine (1.5 g, 8.6 mmol, 1 eq) in DMSO (30 mL) was added NaH (344 mg, 8.6 mmol, 60% purity, 1 eq) at 0° C. under N₂. The mixture was stirred at 25° C. for 4 hours. The reaction mixture was washed with water (100 mL×2), dried over anhydrous Na₂SO₄, filtered and concentrated under reduced pressure. The crude product was purified by flash silica gel chromatography (45% methyl tert-butyl ether in petroleum ether) to give trans-4-[(4-bromo-2-pyridyl)oxy]cyclohexanol (1.2 g, 47% yield) as a yellow oil.

### Step 2

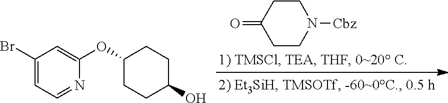 [SMILES: O=C1CCN(C(=O)OCc2ccccc2)CC1 ; O[C@H]1CC[C@H](Oc2cc(Br)ccn2)CC1]

To a stirred solution of trans-4-[(4-bromo-2-pyridyl)oxy]cyclohexanol (1.2 g, 4.4 mmol, 1 eq) in THF (20 mL) were added TEA (491 mg, 4.9 mmol, 0.70 mL, 1.1 eq) and TMSCl (527 mg, 4.9 mmol, 0.60 mL, 1.1 eq) at 0° C., and the reaction mixture was stirred at 20° C. for 0.5 hour. The reaction mixture was filtered and the filtrate was concentrated under reduced pressure to give a residue. Then to a stirred solution of the above residue and benzyl 4-oxopiperidine-1-carboxylate (1.0 g, 4.4 mmol, 0.9 mL, 1.0 eq) in CH₂Cl₂ (50 mL) were added Et₃SiH (564 mg, 4.85 mmol, 0.8 mL, 1.1 eq) and TMSOTf (490 mg, 2.2 mmol, 0.4 mL, 0.5 eq) dropwise at −60° C., and the reaction mixture was stirred at 0° C. under N₂ for 2.5 hours. The reaction mixture was poured into water (50 mL), and pH was adjusted to 7-8 with sat.aq. NaHCO₃. The resulting mixture was extracted with CH₂Cl₂ (50 mL×3). The combined organic layer was washed with brine (30 mL×2), dried over anhydrous Na₂SO₄, filtered and concentrated under reduced pressure. The crude product was purified by flash silica gel chromatography (0-20% ethyl acetate/petroleum ether) to give benzyl 4-[trans-4-[(4-bromo-2-pyridyl)oxy]cyclohexoxy]piperidine-1-carboxylate (1.1 g, 1.7 mmol) as a yellow oil.

Benzyl 4-(((1r, 4r)-4-((4-bromopyridin-2-yl)oxy)cyclohexyl)oxy)piperidine-1-carboxylate was converted to tert-butyl 8-(2-(((1r, 4r)-4-((1-(5-(1-methoxy-3-methyl-1-oxobutan-2-yl)isoxazol-3-yl)piperidin-4-yl)oxy)cyclohexyl)oxy)pyridin-4-yl)-3,8-diazabicyclo[3.2.1]octane-3-carboxylate according to the scheme below using procedures described for other Examples above.

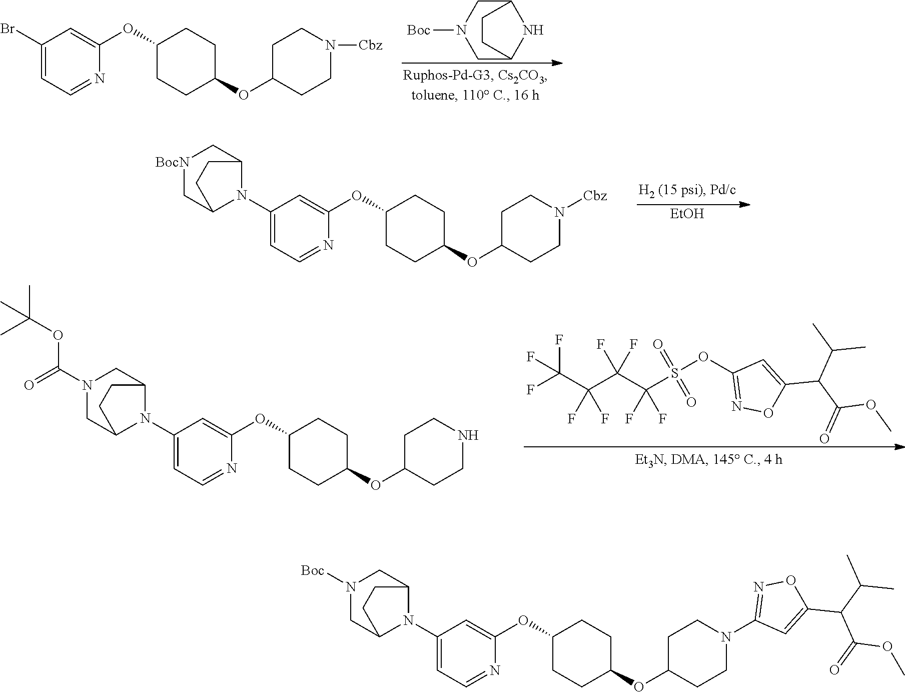

tert-Butyl 8-(2-(((1r, 4r)-4-((1-(5-(1-methoxy-3-methyl-1-oxobutan-2-yl)isoxazol-3-yl)piperidin-4-yl)oxy)cyclohexyl)oxy)pyridin-4-yl)-3,8-diazabicyclo[3.2.1]octane-3-carboxylate was converted to the title compounds as described above for Compound 80.

### Protein Level Control

This description also provides methods for the control of protein levels with a cell. This is based on the use of compounds as described herein, which are known to interact with a specific target protein such that degradation of a target protein in vivo will result in the control of the amount of protein in a biological system, preferably to a particular therapeutic benefit.

The following examples are used to assist in describing the present disclosure, but should not be seen as limiting the present disclosure in any way.

### Specific Embodiments of the Present Disclosure

The present disclosure encompasses the following specific embodiments. These following embodiments may include all of the features recited in a proceeding embodiment, as specified. Where applicable, the following embodiments may also include the features recited in any proceeding embodiment inclusively or in the alternative (e.g., an eighth embodiment may include the features recited in a first embodiment, as recited, and/or the features of any of the second through seventh embodiments).

In any of the aspects or embodiments described herein, the ULM is a ULM as provided in Table 1.

In any of the aspects or embodiments described herein, the compound includes a Linker (L) as described herein, such as a Linker (L) from the compounds of Table 1 (e.g., selected from compounds 1-157).

In any of the aspects or embodiments described herein, the compound is selected from the compounds of Table 1 (e.g., selected from compounds 1-157).

### Assays and Degradation Data

### Western Blot Screen of BRM Degradation in SW1573 Cells

To assess BRM degradation (Dₘₐₓ and DC₅₀) cells were seeded at 8000/well in 96-well black/clear-bottom plates in 180 μL DMEM growth media (containing 1% pen-strep, 1% HEPES and 10% FBS) per well. Plates were incubated overnight to allow adhesion. The next morning cells were treated by adding 20 μL of 10× target compound concentration (1% DMSO) to appropriate wells and returned to incubator for overnight (18-20 hours). The final DMSO concentration was 0.1%.

For lysing, adherent cells were washed once with 100 μL of DPBS. Cells were lysed in 40 μL of 1×RIPA+HALT protease inhibitor on ice for 10 minutes and frozen until use at −80° C. Thawed lysates were cleaned by filtration in 1.2 m filter plates, or alternatively, were spun clean at 2300 g at 4° C. for 30 minutes.

For blotting, for each Western sample 30 μL of lysate was added to 10 μL of 4× LDS sample buffer, then denatured at 95° C. for 5 minutes in the thermal cycler and placed on ice. Samples were loaded on 4-15% Tris/Glycine gels and run for 25 minutes at 250 constant volts in 1× Tris/Glycine buffer with 4 μL of ladder and 12 μL of each sample loaded for each blot. Protein was transferred from gels to NC with BioRad Turbo dry-transfer unit with the Turbo/midi default program. All blots were rinsed with ddH2O and blocked for 1 hour at room temperature in 5% BSA in TBST (0.1%) on rocker. Blots were exposed to primary antibody in 5% BSA in TBST (0.1%) overnight at 4° C. on rocker (1:1000 for BRM (Cell Signaling Tech. cat #11966) and 1:2000 for alpha-tubulin (Cell Signaling Tech. cat #3873), a control protein. Blot was washed with TBST (0.1%) three times for 5 minutes on rocker at room temperature. Secondary antibody was added, and blots were incubated on a room temperature rocker for 1 hour with 1:18,000 anti-rabbit-HRP and/or anti-mouse-HRP in 5% BSA in TBST (0.1%). Blots were washed three times in TBST (0.1%) for 5 minutes at room temperature on the rocker. Signal was developed with Femto Maximum Sensitivity substrate for 4 minutes and blots read on ChemiDoc™.

### In-Cell Western Screen of BRM Degradation in SW1573 Cells

To assess BRM degradation (Dₘₐₓ and DC₅₀), cells were seeded at 8000/well in 96-well black/clear-bottom plates in 180 μL DMEM growth media (containing 1% pen-strep, 1% HEPES and 10% FBS) per well. Plates were incubated overnight to allow adhesion. The next morning cells were treated by adding 20 μL of 10× compound (1% DMSO) to appropriate wells and returned to incubator for overnight (18-20 hours). The final DMSO concentration was 0.1%.

For processing, the plates were removed from incubator, the media was removed, and immediately 200 μL of cold (4° C.) DPBS was added to all wells. DPBS was then removed, and 50 μL of 4% paraformaldehyde (PFA) in DPBS (4° C.) was added to all wells, and the plates were incubated at room temperature for 20 minutes. PFA was then removed, and 200 μL of TBS-T containing 0.5% Triton X-100 was added to all wells, and the plates were incubated at room temperature for 30 minutes. TBS-T containing 0.5% Triton X-100 was then removed, and 50 μL of Li-Cor blocking solution was added, and the plates were incubated at room temperature for a minimum of one hour. The blocking solution was removed, and 50 μL of Li-Cor blocking solution containing primary antibody cocktail was added (1:1000 for BRM (Cell Signaling Tech. cat #11966) and 1:2000 for alpha-tubulin (Sigma cat #T6074), a control protein. Plates were then placed in cold room until the next day.

The next day plates were washed three times with TBS-T, 200 μL per well. Fifty (50) L of secondary antibody cocktail in LI-COR blocking solution (anti-rabbit_800 nm and anti-mouse_680 nm) was added to all wells (diluted 1:5000). The plates were incubated at room temperature for at least one hour while protected from light. The plates were washed twice with TBS-T, 200 μL per well.

To read each plate TBS-T was removed, and each plate was inverted tapped on a paper towel. The plates were read on the LI-COR Odyssey with default intensity settings of 5.0 for both channels. LI-COR images were analyzed with the in-cell Western feature of Image Studio Lite.

The following compounds demonstrated target protein degradation when tested under the conditions described above:

TABLE 1
Exemplary bifunctional degradation compounds of the present disclosure

| Ex. | Structure | Compound Name |
|---|---|---|
| 1 | 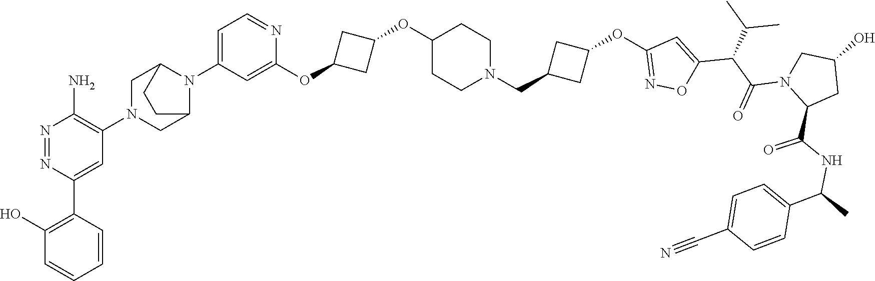 [SMILES: CC(C)[C@H](C(=O)N1C[C@H](O)C[C@H]1C(=O)N[C@@H](C)c1ccc(C#N)cc1)c1cc(O[C@H]2C[C@H](CN3CCC(O[C@H]4C[C@H](Oc5cc(N6C7CCC6CN(c6cc(-c8ccccc8O)nnc6N)C7)ccn5)C4)CC3)C2)no1] | (2S,4R)-N-[(1S)-1-(4-cyanophenyl)ethyl]- 4-hydroxy-1-[(2S)-3-methyl-2-{3-[(1r,3r)- 3-({4-[(1r,3r)-3-[(4-{3-[3-amino-6-(2- hydroxyphenyl)pyridazin-4-yl]-3,8- diazabicyclo[3.2.1]octan-8-yl}pyridin-2- yl)oxy]cyclobutoxy]piperidin-1- yl}methyl)cyclobutoxy]-1,2-oxazol-5- yl}butanoyl]pyrrolidine-2-carboxamide |
| 2 | 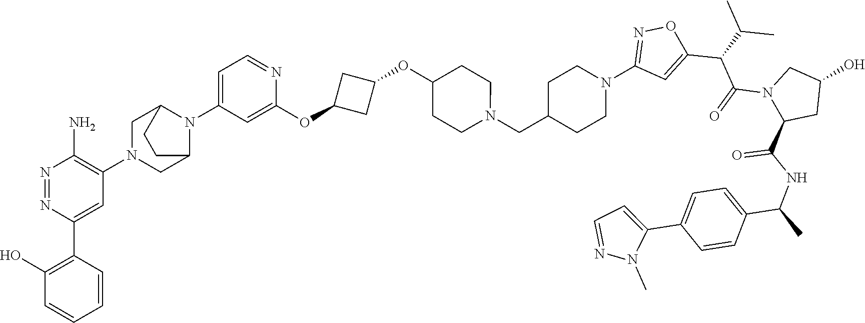 [SMILES: CC(C)[C@H](C(=O)N1C[C@H](O)C[C@H]1C(=O)N[C@@H](C)c1ccc(-c2ccnn2C)cc1)c1cc(N2CCC(CN3CCC(O[C@H]4C[C@H](Oc5cc(N6C7CCC6CN(c6cc(-c8ccccc8O)nnc6N)C7)ccn5)C4)CC3)CC2)no1] | (2S,4R)-4-hydroxy-N-[(1S)-1-[4-(1- methyl-1H-pyrazol-5-yl)phenyl]ethyl]-1- [(2S)-3-methyl-2-{3-[4-({4-[(1r,3r)-3-[(4- {4-[3-amino-6-(2- hydroxyphenyl)pyridazin-4-yl]piperazin- 1-yl}pyridin-2- yl)oxy]cyclobutoxy]piperidin-1- yl}methyl)piperidin-1-yl]-1,2-oxazol-5- yl}butanoyl]pyrrolidine-2-carboxamide |
| 3 | 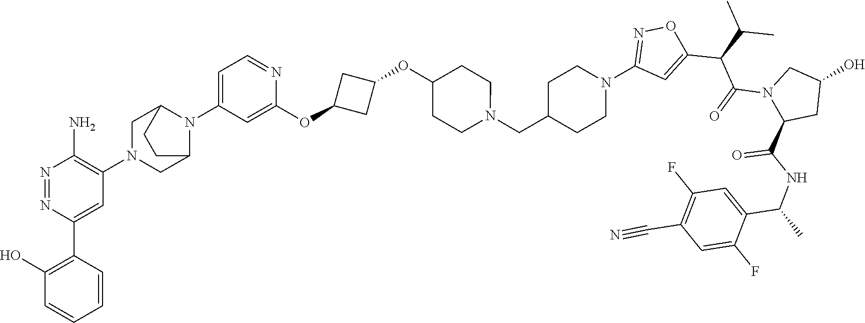 [SMILES: CC(C)[C@@H](C(=O)N1C[C@H](O)C[C@H]1C(=O)N[C@H](C)c1cc(F)c(C#N)cc1F)c1cc(N2CCC(CN3CCC(O[C@H]4C[C@H](Oc5cc(N6C7CCC6CN(c6cc(-c8ccccc8O)nnc6N)C7)ccn5)C4)CC3)CC2)no1] | (2S,4R)-N-[(1R)-1-(4-cyano-2,5- difluorophenyl)ethyl]-4-hydroxy-1-[(2R)- 3-methyl-2-{3-[4-({4-[(1r,3r)-3-[(4-{3-[3- amino-6-(2-hydroxyphenyl)pyridazin-4- yl]-3,8-diazabicyclo[3.2.1]octan-8- yl}pyridin-2- yl)oxy]cyclobutoxy]piperidin-1- yl}methyl)piperidin-1-yl]-1,2-oxazol-5- yl}butanoyl]pyrrolidine-2-carboxamide |
| 4 | 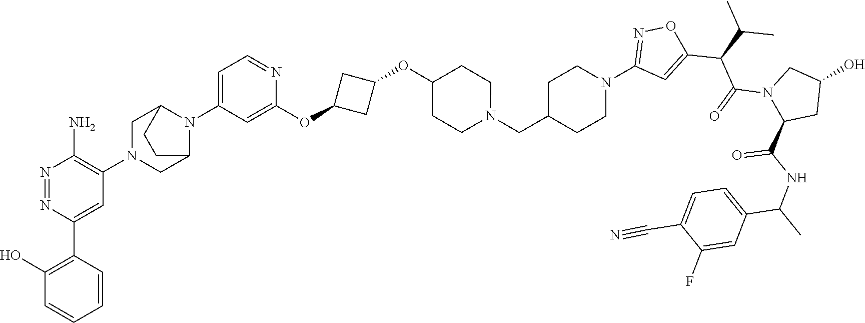 [SMILES: CC(NC(=O)[C@@H]1C[C@@H](O)CN1C(=O)[C@@H](c1cc(N2CCC(CN3CCC(O[C@H]4C[C@H](Oc5cc(N6C7CCC6CN(c6cc(-c8ccccc8O)nnc6N)C7)ccn5)C4)CC3)CC2)no1)C(C)C)c1ccc(C#N)c(F)c1] | (2S,4R)-N-[1-(4-cyano-3- fluorophenyl)ethyl]-4-hydroxy-1-[(2R)-3- methyl-2-{3-[4-({4-[(1r,3r)-3-[(4-{3-[3- amino-6-(2-hydroxyphenyl)pyridazin-4- yl]-3,8-diazabicyclo[3.2.1]octan-8- yl}pyridin-2- yl)oxy]cyclobutoxy]piperidin-1- yl}methyl)piperidin-1-yl]-1,2-oxazol-5- yl}butanoyl]pyrrolidine-2-carboxamide |
| 5 | 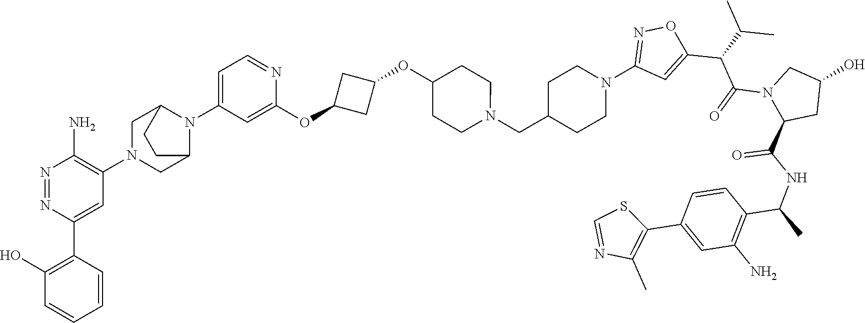 [SMILES: Cc1ncsc1-c1ccc([C@H](C)NC(=O)[C@@H]2C[C@@H](O)CN2C(=O)[C@H](c2cc(N3CCC(CN4CCC(O[C@H]5C[C@H](Oc6cc(N7C8CCC7CN(c7cc(-c9ccccc9O)nnc7N)C8)ccn6)C5)CC4)CC3)no2)C(C)C)c(N)c1] | (2S,4R)-N-[(1S)-1-[2-amino-4-(4-methyl- 1,3-thiazol-5-yl)phenyl]ethyl]-4-hydroxy- 1-[(2S)-3-methyl-2-{3-[4-({4-[(1r,3r)-3- [(4-{3-[3-amino-6-(2- hydroxyphenyl)pyridazin-4-yl]-3,8- diazabicyclo[3.2.1]octan-8-yl}pyridin-2- yl)oxy]cyclobutoxy]piperidin-1- yl}methyl)piperidin-1-yl]-1,2-oxazol-5- yl}butanoyl]pyrrolidine-2-carboxamide |
| 6 | 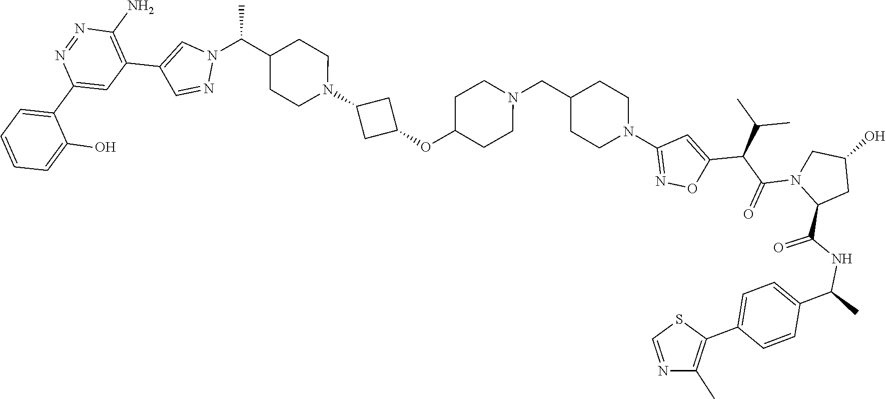 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@@H](N6CCC([C@@H](C)n7cc(-c8cc(-c9ccccc9O)nnc8N)cn7)CC6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1s,3s)-3-{4- [(1R)-1-{4-[3-amino-6-(2- hydroxyphenyl)pyridazin-4-yl]-1H- pyrazol-1-yl}ethyl]piperidin-1- yl}cyclobutoxy]piperidin-1- yl}methyl)piperidin-1-yl]-1,2-oxazol-5- yl}butanoyl]pyrrolidine-2-carboxamide |
| 7 | 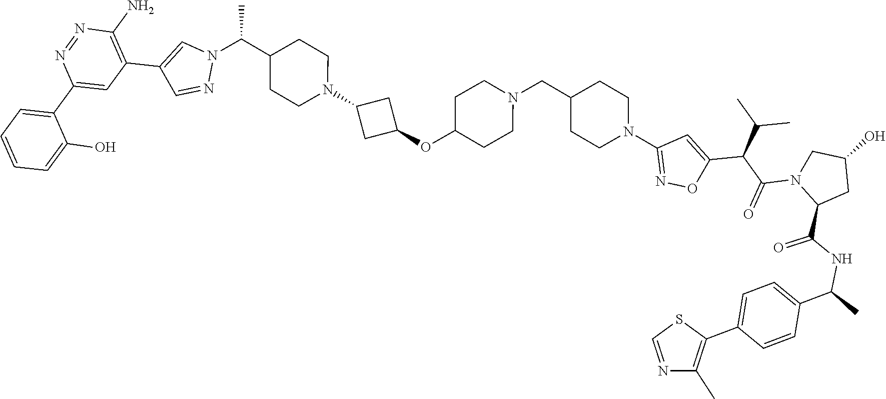 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCC([C@@H](C)n7cc(-c8cc(-c9ccccc9O)nnc8N)cn7)CC6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3-{4- [(1R)-1-{4-[3-amino-6-(2- hydroxyphenyl)pyridazin-4-yl]-1H- pyrazol-1-yl}ethyl]piperidin-1- yl}cyclobutoxy]piperidin-1- yl}methyl)piperidin-1-yl]-1,2-oxazol-5- yl}butanoyl]pyrrolidine-2-carboxamide |
| 8 | 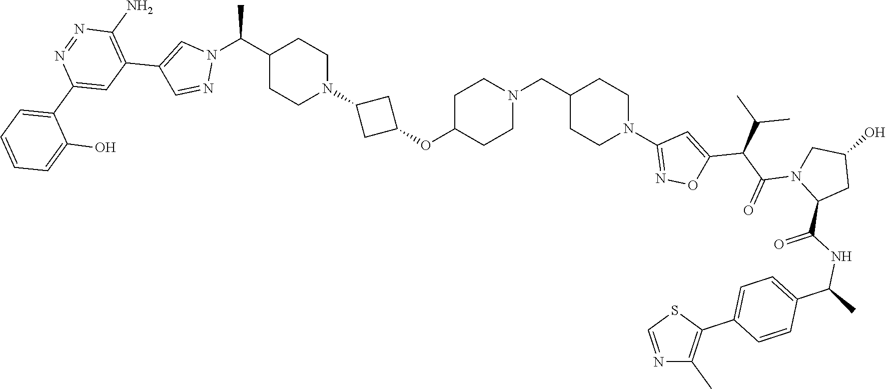 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@@H](N6CCC([C@H](C)n7cc(-c8cc(-c9ccccc9O)nnc8N)cn7)CC6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1s,3s)-3-{4- [(1S)-1-{4-[3-amino-6-(2- hydroxyphenyl)pyridazin-4-yl]-1H- pyrazol-1-yl}ethyl]piperidin-1- yl}cyclobutoxy]piperidin-1- yl}methyl)piperidin-1-yl]-1,2-oxazol-5- yl}butanoyl]pyrrolidine-2-carboxamide |
| 9 | 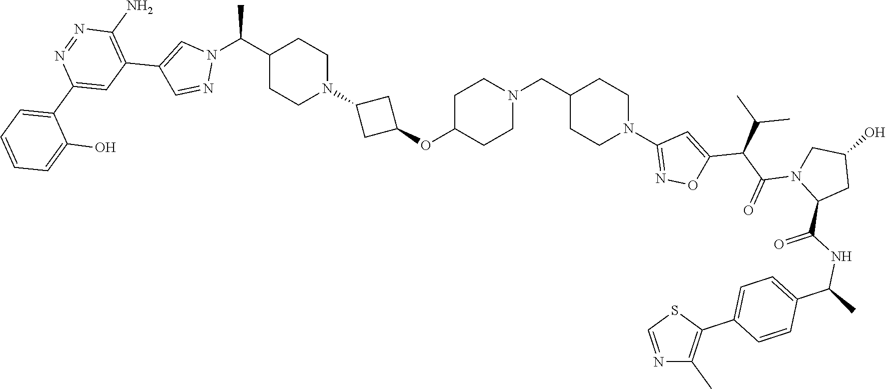 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCC([C@H](C)n7cc(-c8cc(-c9ccccc9O)nnc8N)cn7)CC6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3-{4- [(1S)-1-{4-[3-amino-6-(2- hydroxyphenyl)pyridazin-4-yl]-1H- pyrazol-1-yl}ethyl]piperidin-1- yl}cyclobutoxy]piperidin-1- yl}methyl)piperidin-1-yl]-,2-oxazol-5- yl}butanoyl]pyrrolidine-2-carboxamide |
| 10 | 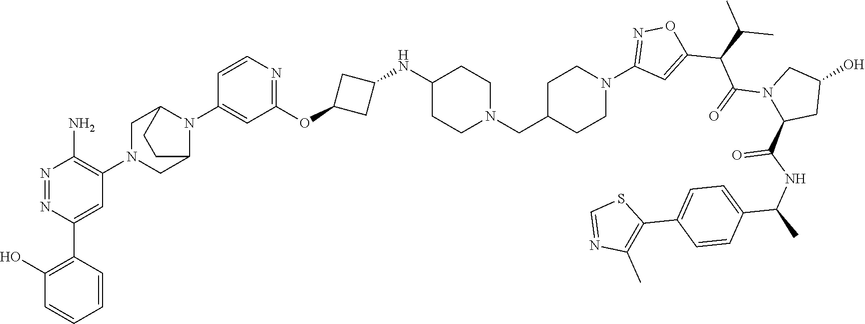 [SMILES: Cc1ncsc1-c1ccc([C@H](C)NC(=O)[C@@H]2C[C@@H](O)CN2C(=O)[C@@H](c2cc(N3CCC(CN4CCC(N[C@H]5C[C@H](Oc6cc(N7C8CCC7CN(c7cc(-c9ccccc9O)nnc7N)C8)ccn6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-(3-{4-[(4-{[(1rs,3rs)-3- [(4-{3-[3-amino-6-(2- hydroxyphenyl)pyridazin-4-yl]-3,8- diazabicyclo[3.2.1]octan-8-yl}pyridin-2- yl)oxy]cyclobutyl]amino}piperidin-1- yl)methyl]piperidin-1-yl}-1,2-oxazol-5- yl)butanoyl]pyrrolidine-2-carboxamide |
| 11 | 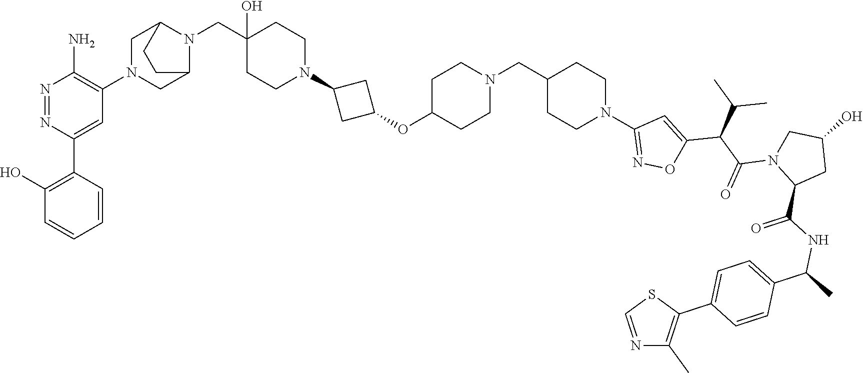 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCC(O)(CN7C8CCC7CN(c7cc(-c9ccccc9O)nnc7N)C8)CC6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3-[4- ({3-[3-amino-6-(2- hydroxyphenyl)pyridazin-4-yl]-3,8- diazabicyclo[3.2.1]octan-8-yl}methyl)-4- hydroxypiperidin-1- yl]cyclobutoxy]piperidin-1- yl}methyl)piperidin-1-yl]-1,2-oxazol-5- yl}butanoyl]pyrrolidine-2-carboxamide |
| 12 | 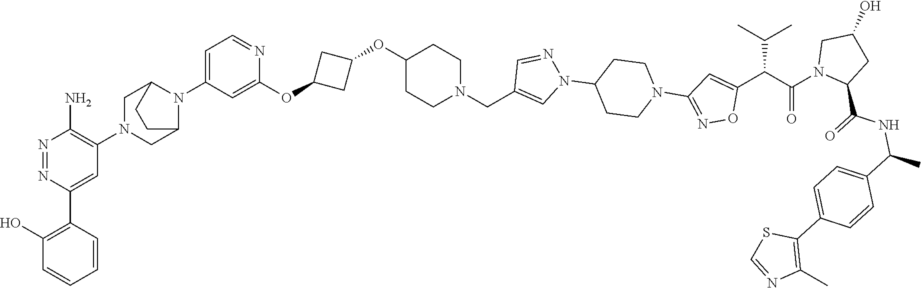 [SMILES: Cc1ncsc1-c1ccc([C@H](C)NC(=O)[C@@H]2C[C@@H](O)CN2C(=O)[C@H](c2cc(N3CCC(n4cc(CN5CCC(O[C@H]6C[C@H](Oc7cc(N8C9CCC8CN(c8cc(-c%10ccccc%10O)nnc8N)C9)ccn7)C6)CC5)cn4)CC3)no2)C(C)C)cc1] | (2S,4R)-4-hydroxy-N-[(1S)-1-[4-(4- methyl-1,3-thiazol-5-yl)phenyl]ethyl]-1- [(2S)-3-methyl-2-(3-{4-[4-({4-[(1r,3r)-3- [(4-{3-[3-amino-6-(2- hydroxyphenyl)pyridazin-4-yl]-3,8- diazabicyclo[3.2.1]octan-8-yl}pyridin-2- yl)oxy]cyclobutoxy]piperidin-1- yl}methyl)-1H-imidazol-1-yl]piperidin-1- yl}-1,2-oxazol-5-yl)butanoyl]pyrrolidine- 2-carboxamide |
| 13 | 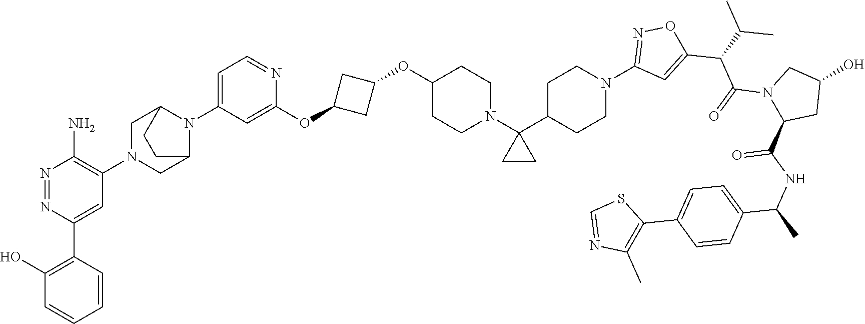 [SMILES: Cc1ncsc1-c1ccc([C@H](C)NC(=O)[C@@H]2C[C@@H](O)CN2C(=O)[C@H](c2cc(N3CCC(C4(N5CCC(O[C@H]6C[C@H](Oc7cc(N8C9CCC8CN(c8cc(-c%10ccccc%10O)nnc8N)C9)ccn7)C6)CC5)CC4)CC3)no2)C(C)C)cc1] | (2S,4R)-4-hydroxy-N-[(3S)-3-[4-(4- methyl-3,3-thiazol-5-yl)phenyl]ethyl]-3- [(2S)-3-methyl-2-{3-[4-(3-{4-[(1r,3r)-3- [(4-{3-[3-amino-6-(2- hydroxyphenyl)pyridazin-4-yl]-3,8- diazabicyclo[3.2.3]octan-8-yl}pyridin-2- yl)oxy]cyclobutoxy]piperidin-1- yl}cyclopropyl)piperidin-1-yl]-1,2-oxazol- 5-yl}butanoyl]pyrrolidine-2-carboxamide |
| 14 | 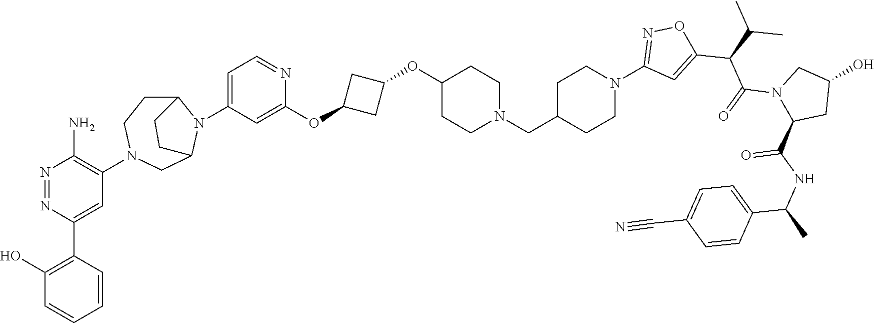 [SMILES: CC(C)[C@@H](C(=O)N1C[C@H](O)C[C@H]1C(=O)N[C@@H](C)c1ccc(C#N)cc1)c1cc(N2CCC(CN3CCC(O[C@H]4C[C@H](Oc5cc(N6C7CCC6CN(c6cc(-c8ccccc8O)nnc6N)CC7)ccn5)C4)CC3)CC2)no1] | (2S,4R)-N-[(1S)-1-(4-cyanophenyl)ethyl]- 4-hydroxy-1-[(2R)-3-methyl-2-{3-[4-({4- [(1r,3r)-3-[(4-{3-[3-amino-6-(2- hydroxyphenyl)pyridazin-4-yl]-3,9- diazabicyclo[4.2.1]nonan-9-yl}pyridin-2- yl)oxy]cyclobutoxy]piperidin-1- yl}methyl)piperidin-1-yl]-1,2-oxazol-5- yl}butanoyl]pyrrolidine-2-carboxamide |
| 15 | 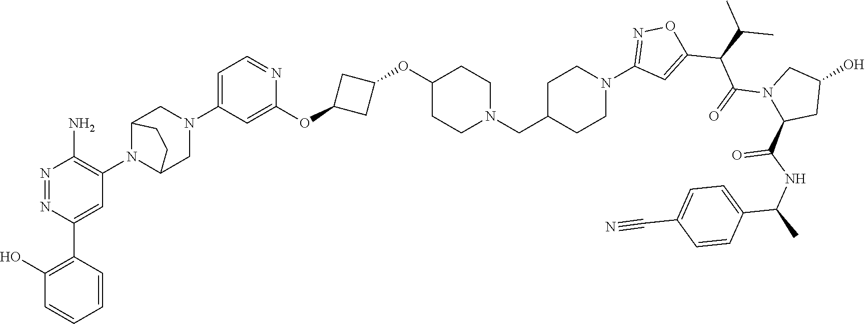 [SMILES: CC(C)[C@@H](C(=O)N1C[C@H](O)C[C@H]1C(=O)N[C@@H](C)c1ccc(C#N)cc1)c1cc(N2CCC(CN3CCC(O[C@H]4C[C@H](Oc5cc(N6CC7CCC(C6)N7c6cc(-c7ccccc7O)nnc6N)ccn5)C4)CC3)CC2)no1] | (2S,4R)-N-[(1S)-1-(4-cyanophenyl)ethyl]- 4-hydroxy-1-[(2R)-3-methyl-2-{3-[4-({4- [(1r,3r)-3-[(4-{8-[3-amino-6-(2- hydroxyphenyl)pyridazin-4-yl]-3,8- diazabicyclo[3.2.1]octan-3-yl}pyridin-2- yl)oxy]cyclobutoxy]piperidin-1- yl}methyl)piperidin-1-yl]-1,2-oxazol-5- yl}butanoyl]pyrrolidine-2-carboxamide |
| 16 | 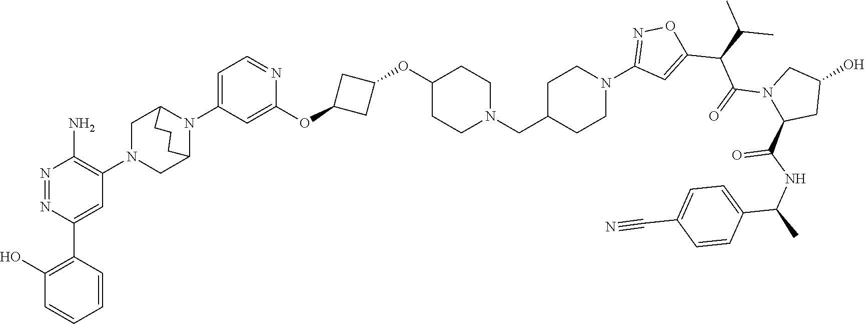 [SMILES: CC(C)[C@@H](C(=O)N1C[C@H](O)C[C@H]1C(=O)N[C@@H](C)c1ccc(C#N)cc1)c1cc(N2CCC(CN3CCC(O[C@H]4C[C@H](Oc5cc(N6C7CCCC6CN(c6cc(-c8ccccc8O)nnc6N)C7)ccn5)C4)CC3)CC2)no1] | (2S,4R)-N-[(1S)-1-(4-cyanophenyl)ethyl]- 4-hydroxy-1-[(2R)-3-methyl-2-{3-[4-({4- [(1r,3r)-3-[(4-{3-[3-amino-6-(2- hydroxyphenyl)pyridazin-4-yl]-3,9- diazabicyclo[3.3.1]nonan-9-yl}pyridin-2- yl)oxy]cyclobutoxy]piperidin-1- yl}methyl)piperidin-1-yl]-1,2-oxazol-5- yl}butanoyl]pyrrolidine-2-carboxamide |
| 17 | 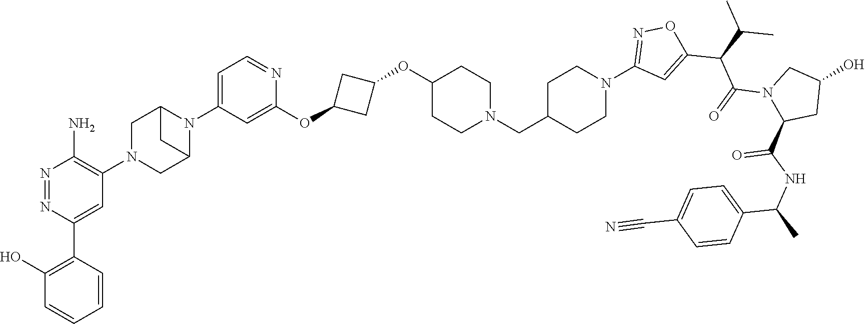 [SMILES: CC(C)[C@@H](C(=O)N1C[C@H](O)C[C@H]1C(=O)N[C@@H](C)c1ccc(C#N)cc1)c1cc(N2CCC(CN3CCC(O[C@H]4C[C@H](Oc5cc(N6C7CC6CN(c6cc(-c8ccccc8O)nnc6N)C7)ccn5)C4)CC3)CC2)no1] | (2S,4R)-N-[(1S)-1-(4-cyanophenyl)ethyl]- 4-hydroxy-1-[(2R)-3-methyl-2-{3-[4-({4- [(1r,3r)-3-[(4-{3-[3-amino-6-(2- hydroxyphenyl)pyridazin-4-yl]-3,6- diazabicyclo[3.1.1]heptan-6-yl}pyridin-2- yl)oxy]cyclobutoxy]piperidin-1- yl}methyl)piperidin-1-yl]-1,2-oxazol-5- yl}butanoyl]pyrrolidine-2-carboxamide |
| 18 | 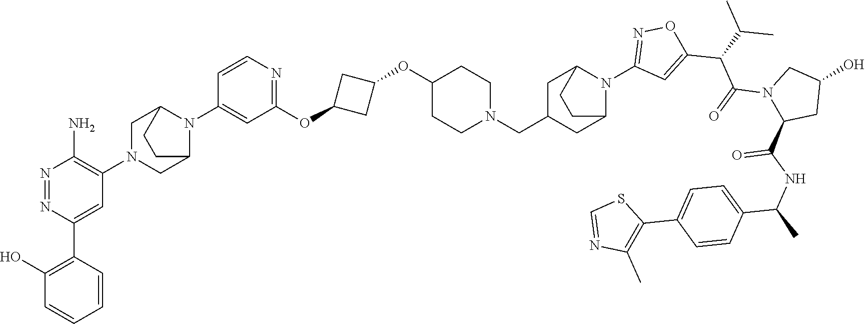 [SMILES: Cc1ncsc1-c1ccc([C@H](C)NC(=O)[C@@H]2C[C@@H](O)CN2C(=O)[C@H](c2cc(N3C4CCC3CC(CN3CCC(O[C@H]5C[C@H](Oc6cc(N7C8CCC7CN(c7cc(-c9ccccc9O)nnc7N)C8)ccn6)C5)CC3)C4)no2)C(C)C)cc1] | (2S,4R)-4-hydroxy-N-[(1S)-1-[4-(4- methyl-1,3-thiazol-5-yl)phenyl]ethyl]-1- [(2S)-3-methyl-2-{3-[3-({4-[(1r*,3r*)-3- [(4-{3-[3-amino-6-(2- hydroxyphenyl)pyridazin-4-yl]-3,8- diazabicyclo[3.2.1]octan-8-yl}pyridin-2- yl)oxy]cyclobutoxy]piperidin-1- yl}methyl)-8-azabicyclo[3.2.1]octan-8- yl]-1,2-oxazol-5-yl}butanoyl]pyrrolidine- 2-carboxamide |
| 19 | 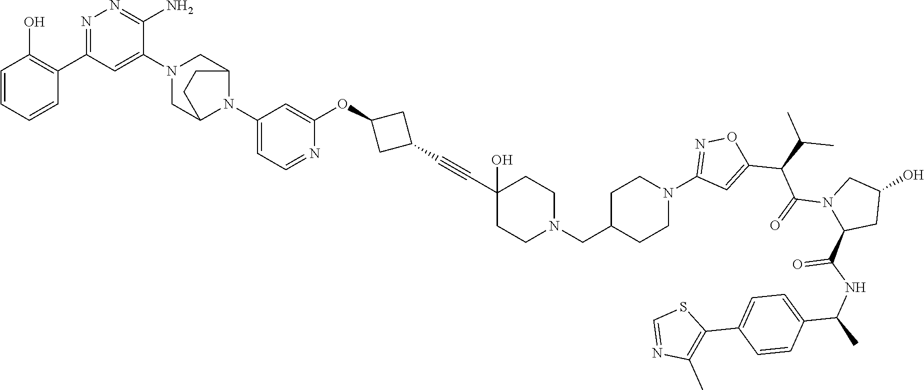 [SMILES: Cc1ncsc1-c1ccc([C@H](C)NC(=O)[C@@H]2C[C@@H](O)CN2C(=O)[C@@H](c2cc(N3CCC(CN4CCC(O)(C#C[C@H]5C[C@H](Oc6cc(N7C8CCC7CN(c7cc(-c9ccccc9O)nnc7N)C8)ccn6)C5)CC4)CC3)no2)C(C)C)cc1] | (2S,4R)-4-hydroxy-1-[(2R)-2-(3-{4-[(4- hydroxy-4-{2-[(1r,3r)-3-[(4-{3-[3-amino- 6-(2-hydroxyphenyl)pyridazin-4-yl]-3,8- diazabicyclo[3.2.1]octan-8-yl}pyridin-2- yl)oxy]cyclobutyl]ethynyl}piperidin-1- yl)methyl]piperidin-1-yl}-1,2-oxazol-5- yl)-3-methylbutanoyl]-N-[(1S)-1-[4-(4- methyl-1,3-thiazol-5- yl)phenyl]ethyl]pyrrolidine-2- carboxamide |
| 20 | 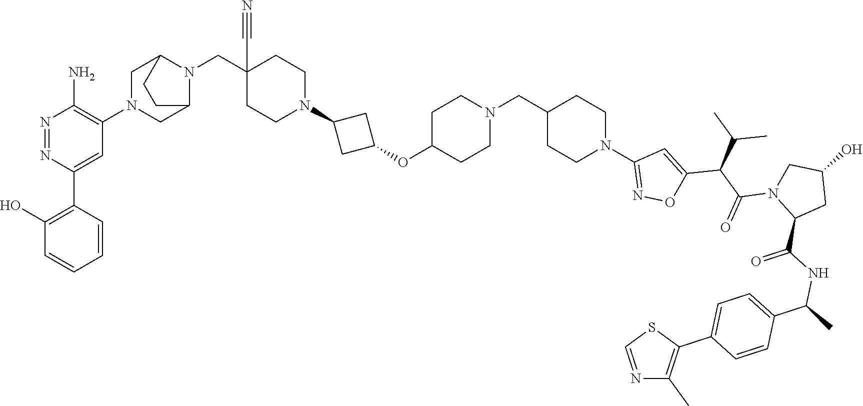 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCC(C#N)(CN7C8CCC7CN(c7cc(-c9ccccc9O)nnc7N)C8)CC6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3-[4- ({3-[3-amino-6-(2- hydroxyphenyl)pyridazin-4-yl]-3,8- diazabicyclo[3.2.1]octan-8-yl}methyl)-4- cyanopiperidin-1- yl]cyclobutoxy]piperidin-1- yl}methyl)piperidin-1-yl]-1,2-oxazol-5- yl}butanoyl]pyrrolidine-2-carboxamide |
| 21 | 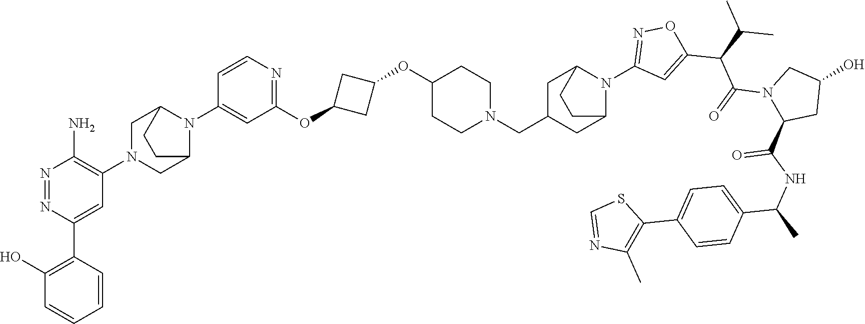 [SMILES: Cc1ncsc1-c1ccc([C@H](C)NC(=O)[C@@H]2C[C@@H](O)CN2C(=O)[C@@H](c2cc(N3C4CCC3CC(CN3CCC(O[C@H]5C[C@H](Oc6cc(N7C8CCC7CN(c7cc(-c9ccccc9O)nnc7N)C8)ccn6)C5)CC3)C4)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[3-({4-[(1r,3r)-3-[(4- {3-[3-amino-6-(2- hydroxyphenyl)pyridazin-4-yl]-3,8- diazabicyclo[3.2.1]octan-8-yl}pyridin-2- yl)oxy]cyclobutoxy]piperidin-1- yl}methyl)-8-azabicyclo[3.2.1]octan-8- yl]-1,2-oxazol-5-yl}butanoyl]pyrrolidine- 2-carboxamide |
| 22 | 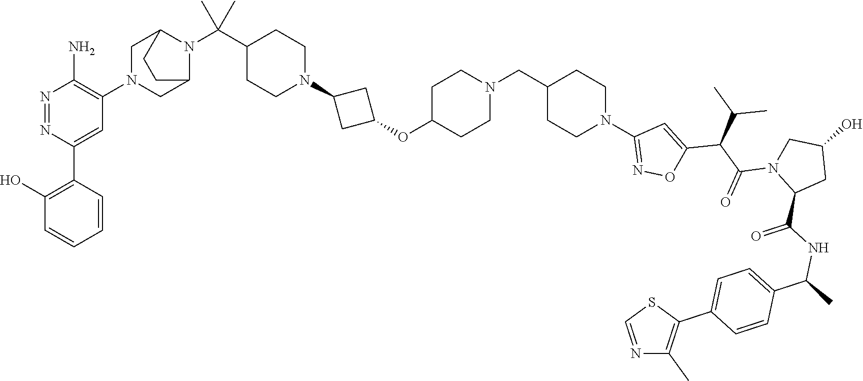 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCC(C(C)(C)N7C8CCC7CN(c7cc(-c9ccccc9O)nnc7N)C8)CC6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3-[4- (2-{3-[3-amino-6-(2- hydroxyphenyl)pyridazin-4-yl]-3,8- diazabicyclo[3.2.1]octan-8-yl}propan-2- yl)piperidin-1-yl]cyclobutoxy]piperidin-1- yl}methyl)piperidin-1-yl]-1,2-oxazol-5- yl}butanoyl]pyrrolidine-2-carboxamide |
| 23 | 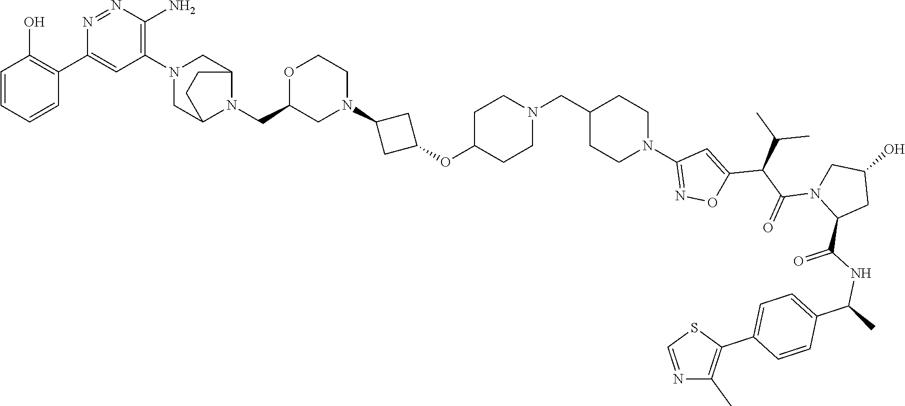 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCO[C@H](CN7C8CCC7CN(c7cc(-c9ccccc9O)nnc7N)C8)C6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3- [(2S)-2-({3-[3-amino-6-(2- hydroxyphenyl)pyridazin-4-yl]-3,8- diazabicyclo[3.2.1]octan-8- yl}methyl)morpholin-4- yl]cyclobutoxy]piperidin-1- yl}methyl)piperidin-1-yl]-1,2-oxazol-5- yl}butanoyl]pyrrolidine-2-carboxamide |
| 24 | 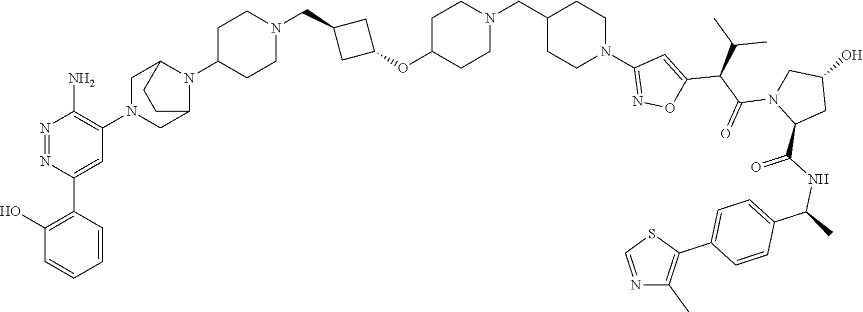 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](CN6CCC(N7C8CCC7CN(c7cc(-c9ccccc9O)nnc7N)C8)CC6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3-[(4- {3-[3-amino-6-(2- hydroxyphenyl)pyridazin-4-yl]-3,8- diazabicyclo[3.2.1]octan-8-yl}piperidin-1- yl)methyl]cyclobutoxy]piperidin-1- yl}methyl)piperidin-1-yl]-1,2-oxazol-5- yl}butanoyl]pyrrolidine-2-carboxamide |
| 25 | 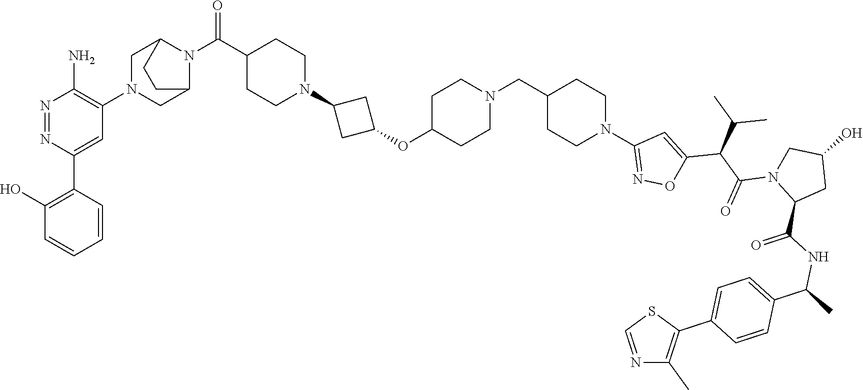 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCC(C(=O)N7C8CCC7CN(c7cc(-c9ccccc9O)nnc7N)C8)CC6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3-(4- {3-[3-amino-6-(2- hydroxyphenyl)pyridazin-4-yl]-3,8- diazabicyclo[3.2.1]octane-8- carbonyl}piperidin-1- yl)cyclobutoxy]piperidin-1- yl}methyl)piperidin-1-yl]-1,2-oxazol-5- yl}butanoyl]pyrrolidine-2-carboxamide |
| 26 | 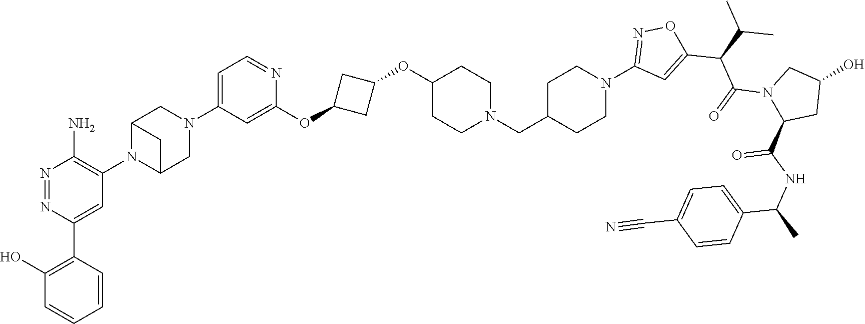 [SMILES: CC(C)[C@@H](C(=O)N1C[C@H](O)C[C@H]1C(=O)N[C@@H](C)c1ccc(C#N)cc1)c1cc(N2CCC(CN3CCC(O[C@H]4C[C@H](Oc5cc(N6CC7CC(C6)N7c6cc(-c7ccccc7O)nnc6N)ccn5)C4)CC3)CC2)no1] | (2S,4R)-N-[(1S)-1-(4-cyanophenyl)ethyl]- 4-hydroxy-1-[(2R)-3-methyl-2-{3-[4-({4- [(1r,3r)-3-[(4-{6-[3-amino-6-(2- hydroxyphenyl)pyridazin-4-yl]-3,6- diazabicyclo[3.1.1]heptan-3-yl}pyridin-2- yl)oxy]cyclobutoxy]piperidin-1- yl}methyl)piperidin-1-yl]-1,2-oxazol-5- yl}butanoyl]pyrrolidine-2-carboxamide |
| 27 | 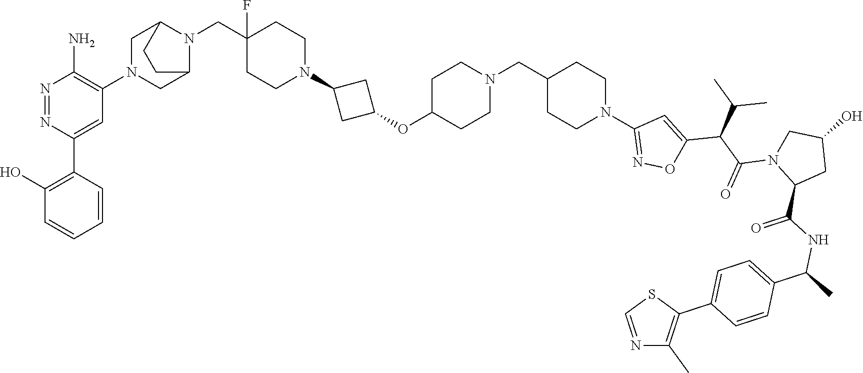 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCC(F)(CN7C8CCC7CN(c7cc(-c9ccccc9O)nnc7N)C8)CC6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3-[4- ({3-[3-amino-6-(2- hydroxyphenyl)pyridazin-4-yl]-3,8- diazabicyclo[3.2.1]octan-8-yl}methyl)-4- fluoropiperidin-1- yl]cyclobutoxy]piperidin-1- yl}methyl)piperidin-1-yl]-1,2-oxazol-5- yl}butanoyl]pyrrolidine-2-carboxamide |
| 28 | 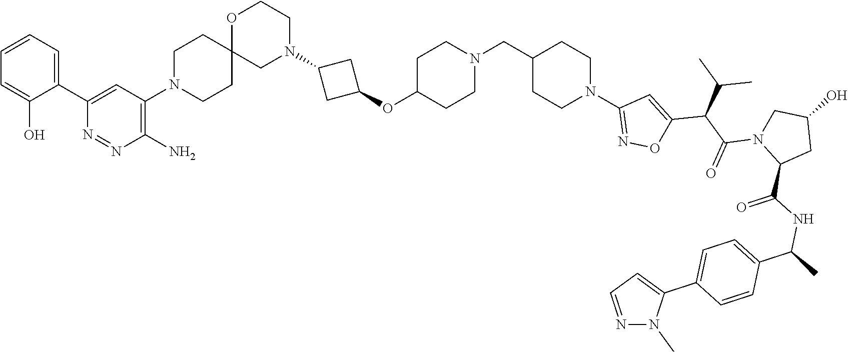 [SMILES: CC(C)[C@@H](C(=O)N1C[C@H](O)C[C@H]1C(=O)N[C@@H](C)c1ccc(-c2ccnn2C)cc1)c1cc(N2CCC(CN3CCC(O[C@H]4C[C@H](N5CCOC6(CCN(c7cc(-c8ccccc8O)nnc7N)CC6)C5)C4)CC3)CC2)no1] | (2S,4R)-4-hydroxy-N-[(1S)-1-[4-(1- methyl-1H-pyrazol-5-yl)phenyl]ethyl]-1- [(2R)-3-methyl-2-{3-[4-({4-[(1r,3r)-3-{9- [3-amino-6-(2-hydroxyphenyl)pyridazin- 4-yl]-1-oxa-4,9-diazaspiro[5.5]undecan-4- yl}cyclobutoxy]piperidin-1- yl}methyl)piperidin-1-yl]-1,2-oxazol-5- yl}butanoyl]pyrrolidine-2-carboxamide |
| 29 | 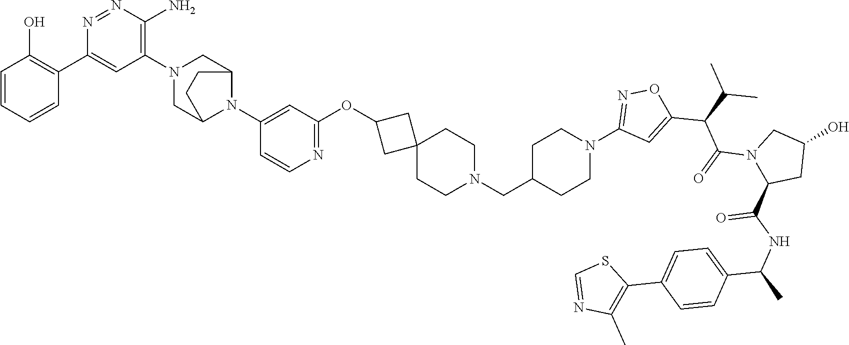 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(Oc4cc(N6C7CCC6CN(c6cc(-c8ccccc8O)nnc6N)C7)ccn4)C5)CC3)no2)C(C)C)cc1] | (2S,4R)-1-[(2R)-2-{3-[4-({2-[(4-{3-[3- amino-6-(2-hydroxyphenyl)pyridazin-4- yl]-3,8-diazabicyclo[3.2.1]octan-8- yl}pyridin-2-yl)oxy]-7- azaspiro[3.5]nonan-7-yl}methyl)piperidin- 1-yl]-1,2-oxazol-5-yl}-3-methylbutanoyl]- 4-hydroxy-N-[(1S)-1-[4-(4-methyl-1,3- thiazol-5-yl)phenyl]ethyl]pyrrolidine-2- carboxamide |
| 30 | 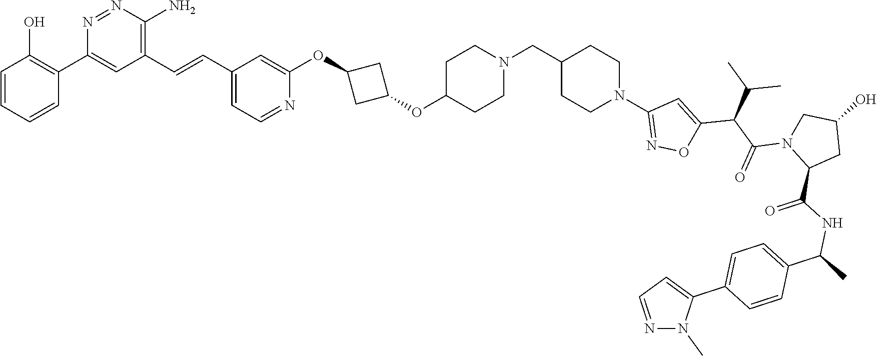 [SMILES: CC(C)[C@@H](C(=O)N1C[C@H](O)C[C@H]1C(=O)N[C@@H](C)c1ccc(-c2ccnn2C)cc1)c1cc(N2CCC(CN3CCC(O[C@H]4C[C@H](Oc5cc(/C=C/c6cc(-c7ccccc7O)nnc6N)ccn5)C4)CC3)CC2)no1] | (2S,4R)-4-hydroxy-N-[(1S)-1-[4-(1- methyl-1H-pyrazol-5-yl)phenyl]ethyl]-1- [(2R)-3-methyl-2-{3-[4-({4-[(1r,3r)-3-({4- [(1E)-2-[3-amino-6-(2- hydroxyphenyl)pyridazin-4- yl]ethenyl]pyridin-2- yl}oxy)cyclobutoxy]piperidin-1- yl}methyl)piperidin-1-yl]-1,2-oxazol-5- yl}butanoyl]pyrrolidine-2-carboxamide |
| 31 | 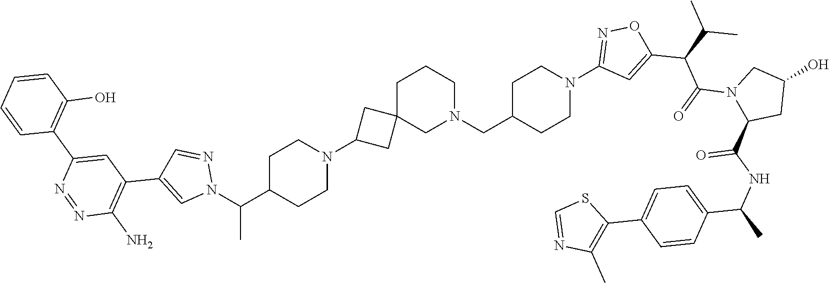 [SMILES: Cc1ncsc1-c1ccc([C@H](C)NC(=O)[C@@H]2C[C@@H](O)CN2C(=O)[C@@H](c2cc(N3CCC(CN4CCCC5(CC(N6CCC(C(C)n7cc(-c8cc(-c9ccccc9O)nnc8N)cn7)CC6)C5)C4)CC3)no2)C(C)C)cc1] | (2S,4R)-1-[(2R)-2-[3-(4-{[4-({2-[4-(1-{4- [3-amino-6-(2-hydroxyphenyl)pyridazin- 4-yl]-1H-pyrazol-1-yl}ethyl)piperidin-1- yl]-7-azaspiro[3.5]nonan-7- yl}methyl)piperidin-1- yl]methyl}piperidin-1-yl)-1,2-oxazol-5- yl]-3-methylbutanoyl]-4-hydroxy-N-[(1S)- 1-[4-(4-methyl-1,3-thiazol-5- yl)phenyl]ethyl]pyrrolidine-2- carboxamide |
| 32 | 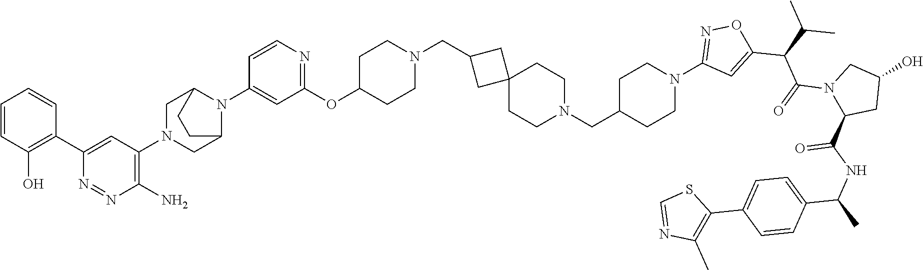 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(CN4CCC(Oc6cc(N7C8CCC7CN(c7cc(-c9ccccc9O)nnc7N)C8)ccn6)CC4)C5)CC3)no2)C(C)C)cc1] | (2S,4R)-1-[(2R)-2-[3-(4-{[2-({4-[(4-{3- [3-amino-6-(2-hydroxyphenyl)pyridazin- 4-yl]-3,8-diazabicyclo[3.2.1]octan-8- yl}pyridin-2-yl)oxy]piperidin-1- yl}methyl)-7-azaspiro[3.5]nonan-7- yl]methyl}piperidin-1-yl)-1,2-oxazol-5- yl]-3-methylbutanoyl]-4-hydroxy-N-[(1S)- 1-[4-(4-methyl-1,3-thiazol-5- yl)phenyl]ethyl]pyrrolidine-2- carboxamide |
| 33 | 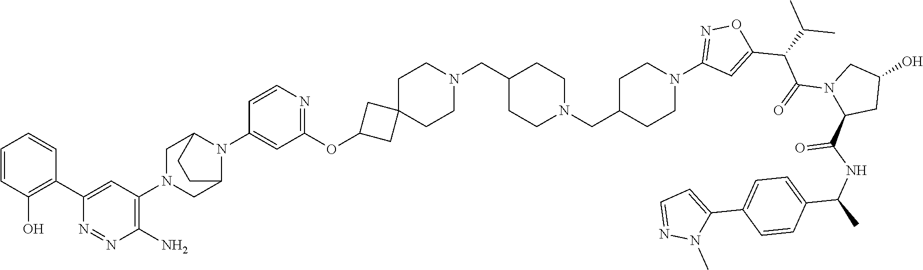 [SMILES: CC(C)[C@H](C(=O)N1C[C@H](O)C[C@H]1C(=O)N[C@@H](C)c1ccc(-c2ccnn2C)cc1)c1cc(N2CCC(CN3CCC(CN4CCC5(CC4)CC(Oc4cc(N6C7CCC6CN(c6cc(-c8ccccc8O)nnc6N)C7)ccn4)C5)CC3)CC2)no1] | (2S,4R)-1-[(2S)-2-[3-(4-{[4-({2-[(4-{3-[3- amino-6-(2-hydroxyphenyl)pyridazin-4- yl]-3,8-diazabicyclo[3.2.1]octan-8- yl}pyridin-2-yl)oxy]-7- azaspiro[3.5]nonan-7-yl}methyl)piperidin- 1-yl]methyl}piperidin-1-yl)-1,2-oxazol-5- yl]-3-methylbutanoyl]-4-hydroxy-N-[(1S)- 1-[4-(1-methyl-1H-pyrazol-5- yl)phenyl]ethyl]pyrrolidine-2- carboxamide |
| 34 | 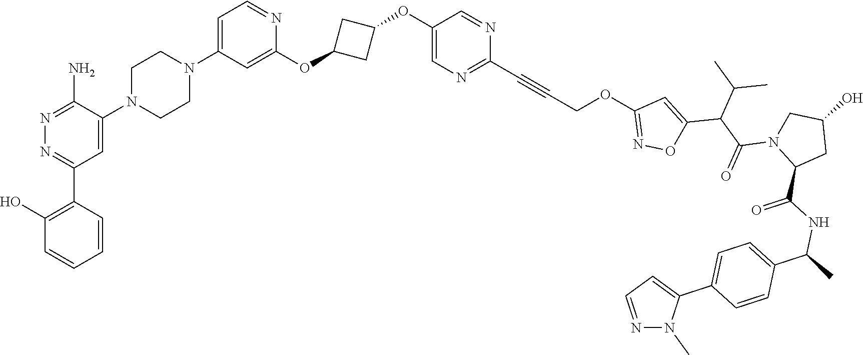 [SMILES: CC(C)C(C(=O)N1C[C@H](O)C[C@H]1C(=O)N[C@@H](C)c1ccc(-c2ccnn2C)cc1)c1cc(OCC#Cc2ncc(O[C@H]3C[C@H](Oc4cc(N5CCN(c6cc(-c7ccccc7O)nnc6N)CC5)ccn4)C3)cn2)no1] | (2S,4R)-4-hydroxy-N-[(1S)-1-[4-(1- methyl-1H-pyrazol-5-yl)phenyl]ethyl]-1- (3-methyl-2-{3-[(3-{5-[(1r,3r)-3-[(4-{4- [3-amino-6-(2-hydroxyphenyl)pyridazin- 4-yl]piperazin-1-yl}pyridin-2- yl)oxy]cyclobutoxy]pyrimidin-2-yl}prop- 2-yn-1-yl)oxy]-1,2-oxazol-5- yl}butanoyl)pyrrolidine-2-carboxamide |
| 35 | 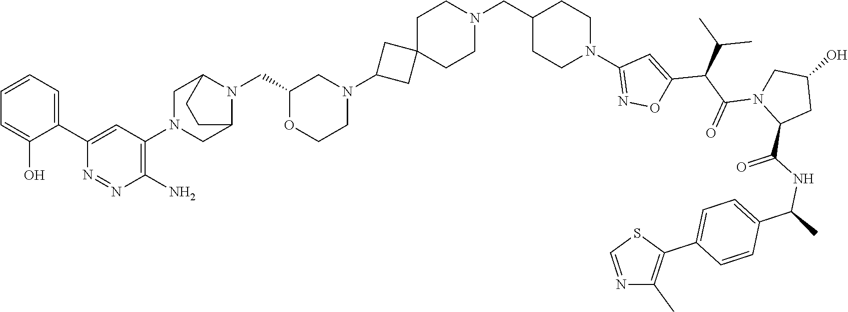 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(N4CCO[C@H](CN6C7CCC6CN(c6cc(-c8ccccc8O)nnc6N)C7)C4)C5)CC3)no2)C(C)C)cc1] | (2S,4R)-1-[(2R)-2-{3-[4-({2-[(2S)-2-({3- [3-amino-6-(2-hydroxyphenyl)pyridazin- 4-yl]-3,8-diazabicyclo[3.2.1]octan-8- yl}methyl)morpholin-4-yl]-7- azaspiro[3.5]nonan-7-yl}methyl)piperidin- 1-yl]-1,2-oxazol-5-yl}-3-methylbutanoyl]- 4-hydroxy-N-[(1S)-1-[4-(4-methyl-1,3- thiazol-5-yl)phenyl]ethyl]pyrrolidine-2- carboxamide |
| 36 | 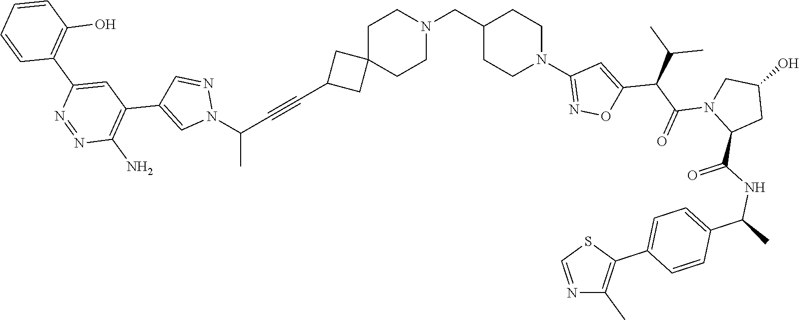 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(C#CC(C)n4cc(-c6cc(-c7ccccc7O)nnc6N)cn4)C5)CC3)no2)C(C)C)cc1] | (2S,4R)-1-[(2R)-2-[3-(4-{[2-(3-{4-[3- amino-6-(2-hydroxyphenyl)pyridazin-4- yl]-1H-pyrazol-1-yl}but-1-yn-1-yl)-7- azaspiro[3.5]nonan-7-yl]methyl}piperidin- 1-yl)-1,2-oxazol-5-yl]-3-methylbutanoyl]- 4-hydroxy-N-[(1S)-1-[4-(4-methyl-1,3- thiazol-5-yl)phenyl]ethyl]pyrrolidine-2- carboxamide |
| 37 | 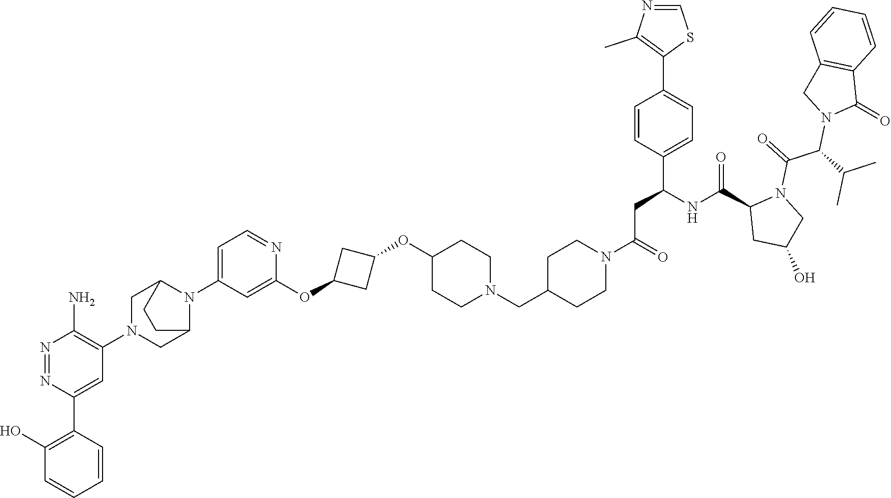 [SMILES: Cc1ncsc1-c1ccc([C@H](CC(=O)N2CCC(CN3CCC(O[C@H]4C[C@H](Oc5cc(N6C7CCC6CN(c6cc(-c8ccccc8O)nnc6N)C7)ccn5)C4)CC3)CC2)NC(=O)[C@@H]2C[C@@H](O)CN2C(=O)[C@@H](C(C)C)N2Cc3ccccc3C2=O)cc1] | (2S,4R)-4-hydroxy-N-[(1S)-1-[4-(4- methyl-1,3-thiazol-5-yl)phenyl]-3-oxo-3- [4-({4-[(1r,3r)-3-[(4-{3-[3-amino-6-(2- hydroxyphenyl)pyridazin-4-yl]-3,8- diazabicyclo[3.2.1]octan-8-yl}pyridin-2- yl)oxy]cyclobutoxy]piperidin-1- yl}methyl)piperidin-1-yl]propyl]-1-[(2R)- 3-methyl-2-(1-oxo-2,3-dihydro-1H- isoindol-2-yl)butanoyl]pyrrolidine-2- carboxamide |
| 38 | 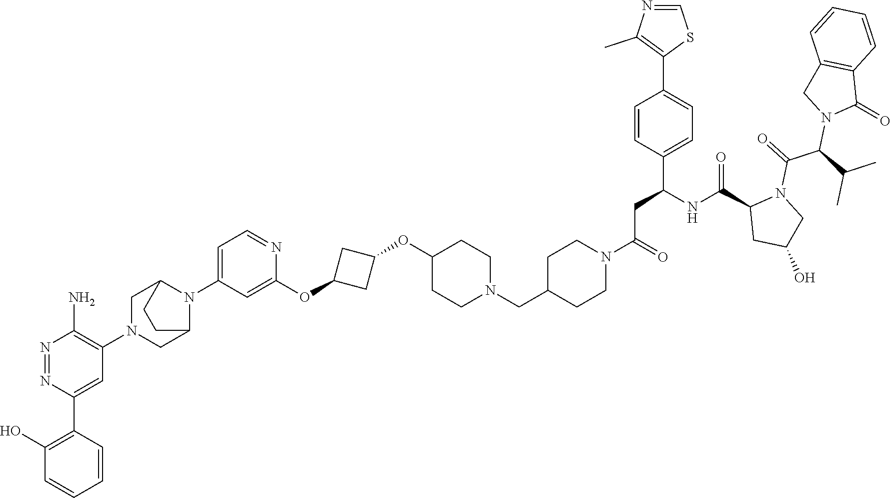 [SMILES: Cc1ncsc1-c1ccc([C@H](CC(=O)N2CCC(CN3CCC(O[C@H]4C[C@H](Oc5cc(N6C7CCC6CN(c6cc(-c8ccccc8O)nnc6N)C7)ccn5)C4)CC3)CC2)NC(=O)[C@@H]2C[C@@H](O)CN2C(=O)[C@H](C(C)C)N2Cc3ccccc3C2=O)cc1] | (2S,4R)-4-hydroxy-N-[(1S)-1-[4-(4- methyl-1,3-thiazol-5-yl)phenyl]-3-oxo-3- [4-({4-[(1r,3r)-3-[(4-{3-[3-amino-6-(2- hydroxyphenyl)pyridazin-4-yl]-3,8- diazabicyclo[3.2.1]octan-8-yl}pyridin-2- yl)oxy]cyclobutoxy]piperidin-1- yl}methyl)piperidin-1-yl]propyl]-1-[(2S)- 3-methyl-2-(1-oxo-2,3-dihydro-1H- isoindol-2-yl)butanoyl]pyrrolidine-2- carboxamide |
| 39 | 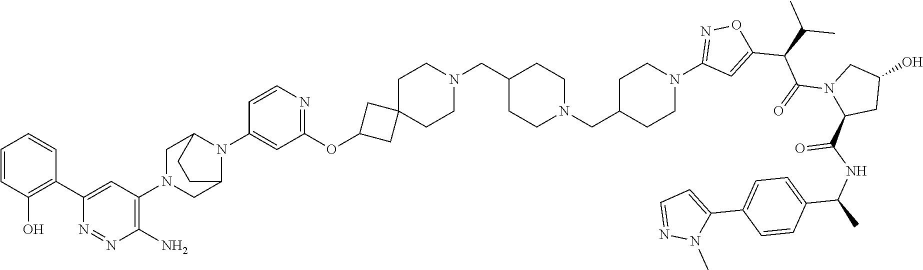 [SMILES: CC(C)[C@@H](C(=O)N1C[C@H](O)C[C@H]1C(=O)N[C@@H](C)c1ccc(-c2ccnn2C)cc1)c1cc(N2CCC(CN3CCC(CN4CCC5(CC4)CC(Oc4cc(N6C7CCC6CN(c6cc(-c8ccccc8O)nnc6N)C7)ccn4)C5)CC3)CC2)no1] | (2S,4R)-1-[(2R)-2-[3-(4-{[4-({2-[(4-{3- [3-amino-6-(2-hydroxyphenyl)pyridazin- 4-yl]-3,8-diazabicyclo[3.2.1]octan-8- yl}pyridin-2-yl)oxy]-7- azaspiro[3.5]nonan-7-yl}methyl)piperidin- 1-yl]methyl}piperidin-1-yl)-1,2-oxazol-5- yl]-3-methylbutanoyl]-4-hydroxy-N-[(1S)- 1-[4-(1-methyl-1H-pyrazol-5- yl)phenyl]ethyl]pyrrolidine-2- carboxamide |
| 40 | 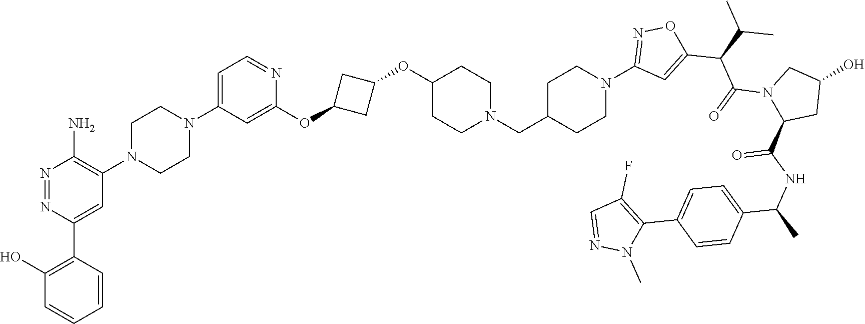 [SMILES: CC(C)[C@@H](C(=O)N1C[C@H](O)C[C@H]1C(=O)N[C@@H](C)c1ccc(-c2c(F)cnn2C)cc1)c1cc(N2CCC(CN3CCC(O[C@H]4C[C@H](Oc5cc(N6CCN(c7cc(-c8ccccc8O)nnc7N)CC6)ccn5)C4)CC3)CC2)no1] | (2S,4R)-N-[(1S)-1-[4-(4-fluoro-1-methyl- 1H-pyrazol-5-yl)phenyl]ethyl]-4-hydroxy- 1-[(2R)-3-methyl-2-{3-[4-({4-[(1r,3r)-3- [(4-{4-[3-amino-6-(2- hydroxyphenyl)pyridazin-4-yl]piperazin- 1-yl}pyridin-2- yl)oxy]cyclobutoxy]piperidin-1- yl}methyl)piperidin-1-yl]-1,2-oxazol-5- yl}butanoyl]pyrrolidine-2-carboxamide |
| 41 | 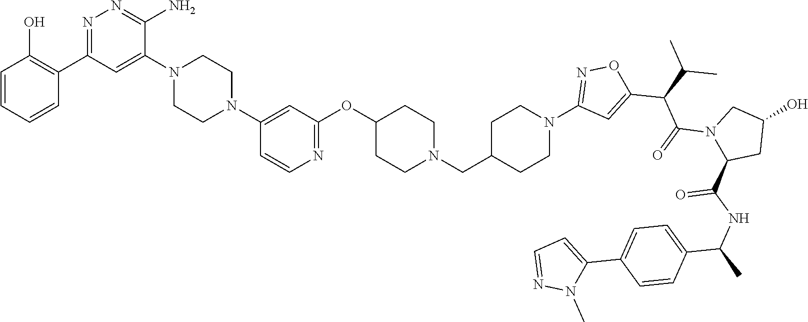 [SMILES: CC(C)[C@@H](C(=O)N1C[C@H](O)C[C@H]1C(=O)N[C@@H](C)c1ccc(-c2ccnn2C)cc1)c1cc(N2CCC(CN3CCC(Oc4cc(N5CCN(c6cc(-c7ccccc7O)nnc6N)CC5)ccn4)CC3)CC2)no1] | (2S,4R)-1-[(2R)-2-{3-[4-({4-[(4-{4-[3- amino-6-(2-hydroxyphenyl)pyridazin-4- yl]piperazin-1-yl}pyridin-2- yl)oxy]piperidin-1-yl}methyl)piperidin-1- yl]-1,2-oxazol-5-yl}-3-methylbutanoyl]-4- hydroxy-N-[(1S)-1-[4-(1-methyl-1H- pyrazol-5-yl)phenyl]ethyl]pyrrolidine-2- carboxamide |
| 42 | 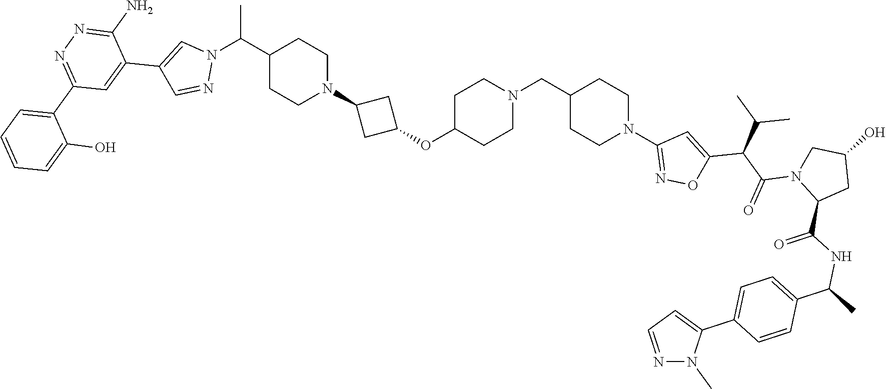 [SMILES: CC(C)[C@@H](C(=O)N1C[C@H](O)C[C@H]1C(=O)N[C@@H](C)c1ccc(-c2ccnn2C)cc1)c1cc(N2CCC(CN3CCC(O[C@H]4C[C@H](N5CCC(C(C)n6cc(-c7cc(-c8ccccc8O)nnc7N)cn6)CC5)C4)CC3)CC2)no1] | (2S,4R)-4-hydroxy-N-[(1S)-1-[4-(1- methyl-1H-pyrazol-5-yl)phenyl]ethyl]-1- [(2R)-3-methyl-2-{3-[4-({4-[(1r,3r)-3-{4- [1-(4-{2-[3-amino-6-(2- hydroxyphenyl)pyridazin-4-yl]ethynyl}- 1H-pyrazol-1-yl)ethyl]piperidin-1- yl}cyclobutoxy]piperidin-1- yl}methyl)piperidin-1-yl]-1,2-oxazol-5- yl}butanoyl]pyrrolidine-2-carboxamide |
| 43 | 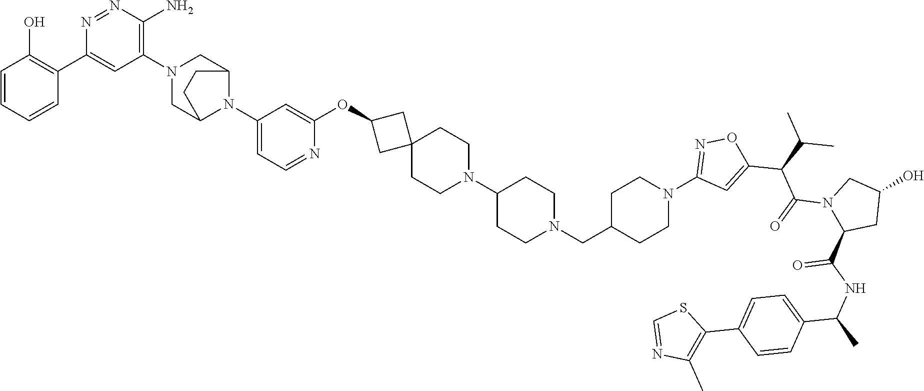 | (2S,4R)-1-[(2R)-2-(3-{4-[(4-{2-[(4-{3-[3- amino-6-(2-hydroxyphenyl)pyridazin-4- yl]-3,8-diazabicyclo[3.2.1]octan-8- yl}pyridin-2-yl)oxy]-7- azaspiro[3.5]nonan-7-yl}piperidin-1- yl)methyl]piperidin-1-yl}-1,2-oxazol-5- yl)-3-methylbutanoyl]-4-hydroxy-N-[(1S)- 1-[4-(4-methyl-1,3-thiazol-5- yl)phenyl]ethyl]pyrrolidine-2- carboxamide |
| 44 | 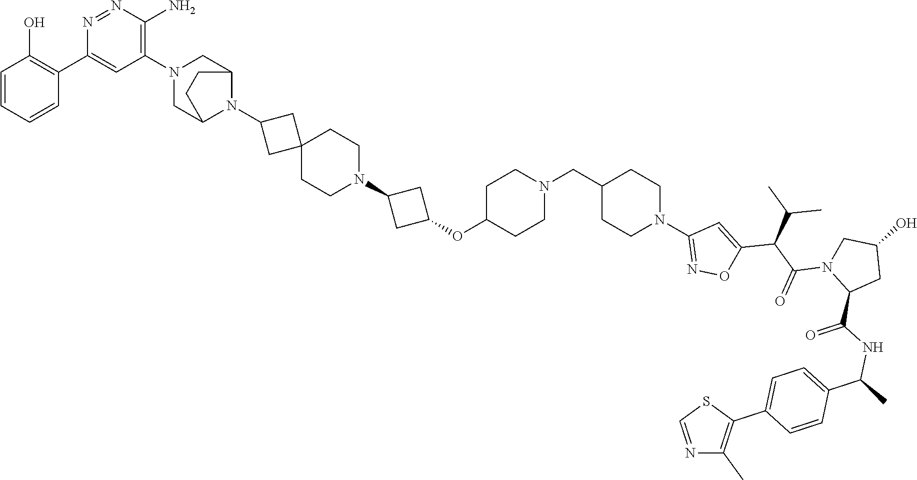 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCC7(CC6)CC(N6C8CCC6CN(c6cc(-c9ccccc9O)nnc6N)C8)C7)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3-(2- {3-[3-amino-6-(2- hydroxyphenyl)pyridazin-4-yl]-3,8- diazabicyclo[3.2.1]octan-8-yl}-7- azaspiro[3.5]nonan-7- yl)cyclobutoxy]piperidin-1- yl}methyl)piperidin-1-yl]-1,2-oxazol-5- yl}butanoyl]pyrrolidine-2-carboxamide |
| 45 | 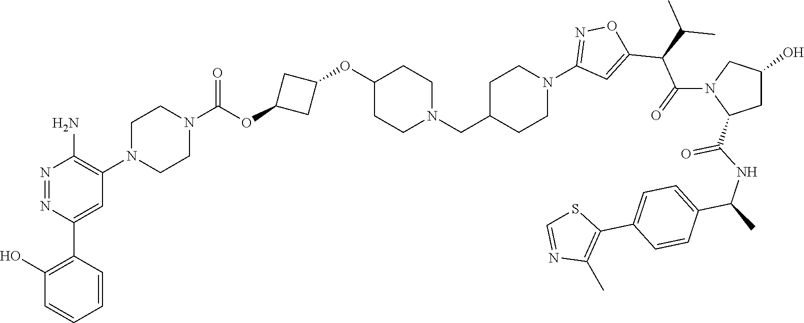 [SMILES: Cc1ncsc1-c1ccc([C@H](C)NC(=O)[C@H]2C[C@@H](O)CN2C(=O)[C@@H](c2cc(N3CCC(CN4CCC(O[C@H]5C[C@H](OC(=O)N6CCN(c7cc(-c8ccccc8O)nnc7N)CC6)C5)CC4)CC3)no2)C(C)C)cc1] | (1rs,3rs)-3-({1-[(1-{5-[(2R)-1-[(2S,4R)-4- hydroxy-2-{[(1S)-1-[4-(4-methyl-1,3- thiazol-5- yl)phenyl]ethyl]carbamoyl}pyrrolidin-1- yl]-3-methyl-1-oxobutan-2-yl]-1,2-oxazol- 3-yl}piperidin-4-yl)methyl]piperidin-4- yl}oxy)cyclobutyl 4-[3-amino-6-(2- hydroxyphenyl)pyridazin-4-yl]piperazine- 1-carboxylate |
| 46 | 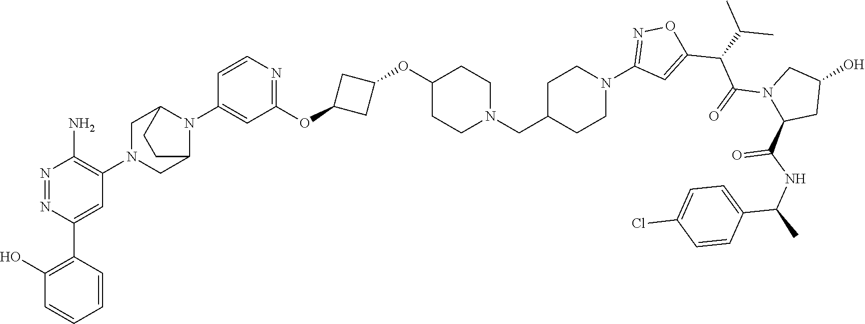 [SMILES: CC(C)[C@H](C(=O)N1C[C@H](O)C[C@H]1C(=O)N[C@@H](C)c1ccc(Cl)cc1)c1cc(N2CCC(CN3CCC(O[C@H]4C[C@H](Oc5cc(N6C7CCC6CN(c6cc(-c8ccccc8O)nnc6N)C7)ccn5)C4)CC3)CC2)no1] | (2S,4R)-N-[(1S)-1-(4-chlorophenyl)ethyl]- 4-hydroxy-1-[(2S)-3-methyl-2-{3-[4-({4- [(1r,3r)-3-[(4-{3-[3-amino-6-(2- hydroxyphenyl)pyridazin-4-yl]-3,8- diazabicyclo[3.2.1]octan-8-yl}pyridin-2- yl)oxy]cyclobutoxy]piperidin-1- yl}methyl)piperidin-1-yl]-1,2-oxazol-5- yl}butanoyl]pyrrolidine-2-carboxamide |
| 47 | 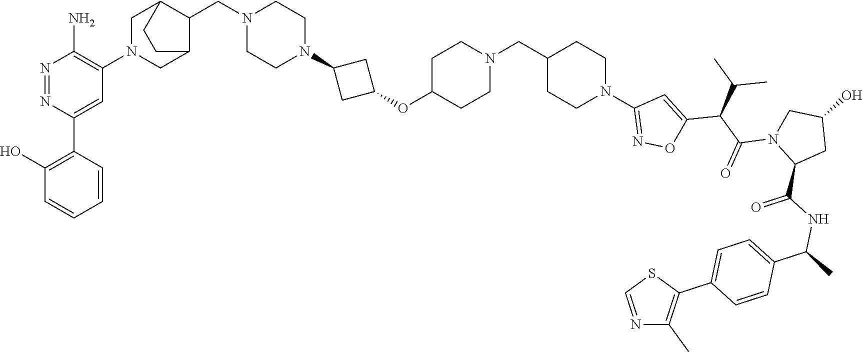 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCN(CC7C8CCC7CN(c7cc(-c9ccccc9O)nnc7N)C8)CC6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3-[4- ({3-[3-amino-6-(2- hydroxyphenyl)pyridazin-4-yl]-3- azabicyclo[3.2.1]octan-8- yl}methyl)piperazin-1- yl]cyclobutoxy]piperidin-1- yl}methyl)piperidin-1-yl]-1,2-oxazol-5- yl}butanoyl]pyrrolidine-2-carboxamide |
| 48 | 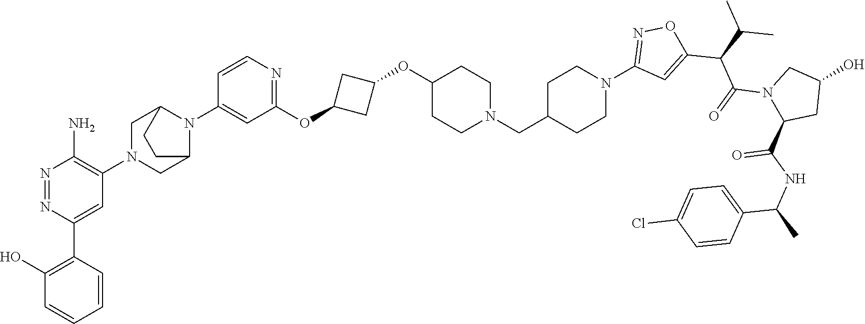 [SMILES: CC(C)[C@@H](C(=O)N1C[C@H](O)C[C@H]1C(=O)N[C@@H](C)c1ccc(Cl)cc1)c1cc(N2CCC(CN3CCC(O[C@H]4C[C@H](Oc5cc(N6C7CCC6CN(c6cc(-c8ccccc8O)nnc6N)C7)ccn5)C4)CC3)CC2)no1] | (2S,4R)-N-[(1S)-1-(4-chlorophenyl)ethyl]- 4-hydroxy-1-[(2R*)-3-methyl-2-{3-[4-({4- [(1rs,3rs)-3-[(4-{3-[3-amino-6-(2- hydroxyphenyl)pyridazin-4-yl]-3,8- diazabicyclo[3.2.1]octan-8-yl}pyridin-2- yl)oxy]cyclobutoxy]piperidin-1- yl}methyl)piperidin-1-yl]-1,2-oxazol-5- yl}butanoyl]pyrrolidine-2-carboxamide |
| 49 | 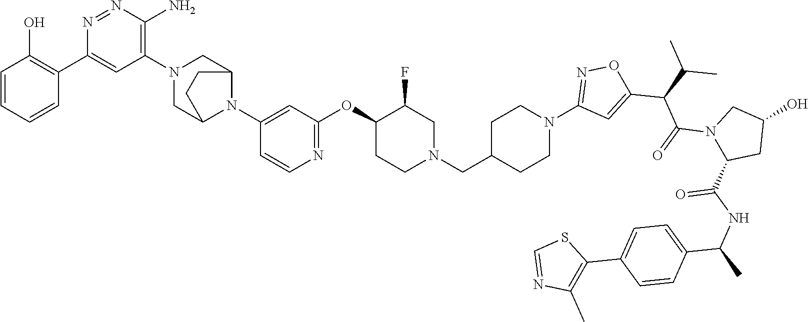 [SMILES: Cc1ncsc1-c1ccc([C@H](C)NC(=O)[C@H]2C[C@@H](O)CN2C(=O)[C@@H](c2cc(N3CCC(CN4CC[C@@H](Oc5cc(N6C7CCC6CN(c6cc(-c8ccccc8O)nnc6N)C7)ccn5)[C@@H](F)C4)CC3)no2)C(C)C)cc1] | (2S,4R)-1-[(2R)-2-[3-(4-{[(3S,4R)-4-[(4- {3-[3-amino-6-(2- hydroxyphenyl)pyridazin-4-yl]-3,8- diazabicyclo[3.2.1]octan-8-yl}pyridin-2- yl)oxy]-3-fluoropiperidin-1- yl]methyl}piperidin-1-yl)-1,2-oxazol-5- yl]-3-methylbutanoyl]-4-hydroxy-N-[(1S)- 1-[4-(4-methyl-1,3-thiazol-5- yl)phenyl]ethyl]pyrrolidine-2- carboxamide |
| 50 | 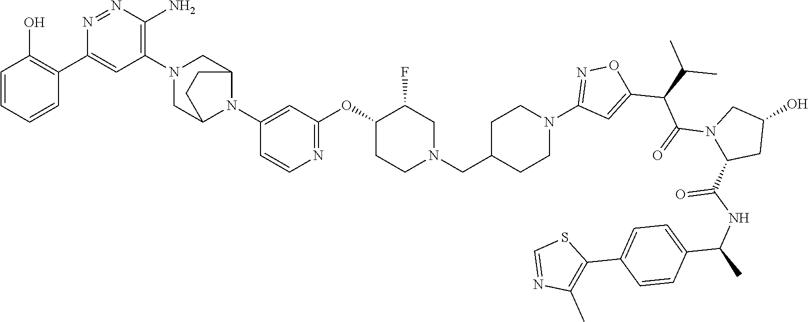 [SMILES: Cc1ncsc1-c1ccc([C@H](C)NC(=O)[C@H]2C[C@@H](O)CN2C(=O)[C@@H](c2cc(N3CCC(CN4CC[C@H](Oc5cc(N6C7CCC6CN(c6cc(-c8ccccc8O)nnc6N)C7)ccn5)[C@H](F)C4)CC3)no2)C(C)C)cc1] | (2S,4R)-1-[(2R)-2-[3-(4-{[(3R,4S)-4-[(4- {3-[3-amino-6-(2- hydroxyphenyl)pyridazin-4-yl]-3,8- diazabicyclo[3.2.1]octan-8-yl}pyridin-2- yl)oxy]-3-fluoropiperidin-1- yl]methyl}piperidin-1-yl)-1,2-oxazol-5- yl]-3-methylbutanoyl]-4-hydroxy-N-[(1S)- 1-[4-(4-methyl-1,3-thiazol-5- yl)phenyl]ethyl]pyrrolidine-2- carboxamide |
| 51 | 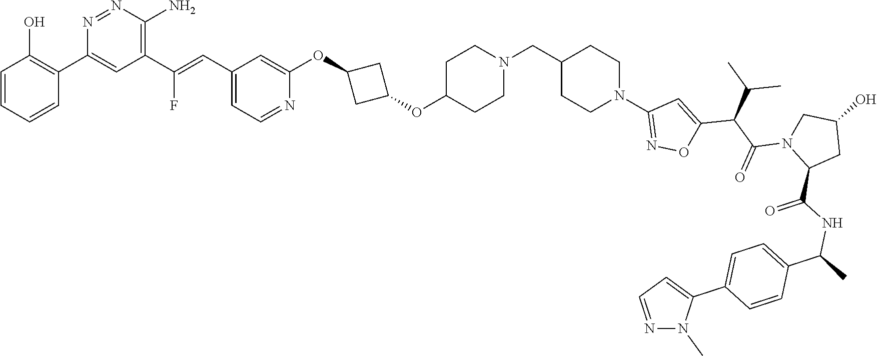 [SMILES: CC(C)[C@@H](C(=O)N1C[C@H](O)C[C@H]1C(=O)N[C@@H](C)c1ccc(-c2ccnn2C)cc1)c1cc(N2CCC(CN3CCC(O[C@H]4C[C@H](Oc5cc(/C=C(\F)c6cc(-c7ccccc7O)nnc6N)ccn5)C4)CC3)CC2)no1] | (2S,4R)-4-hydroxy-N-[(1S)-1-[4-(1- methyl-1H-pyrazol-5-yl)phenyl]ethyl]-1- [(2R)-3-methyl-2-{3-[4-({4-[(1r,3r)-3-[(4- {2-[3-amino-6-(2- hydroxyphenyl)pyridazin-4-yl]-2- fluoroethenyl}pyridin-2- yl)oxy]cyclobutoxy]piperidin-1- yl}methyl)piperidin-1-yl]-1,2-oxazol-5- yl}butanoyl]pyrrolidine-2-carboxamide |
| 52 | 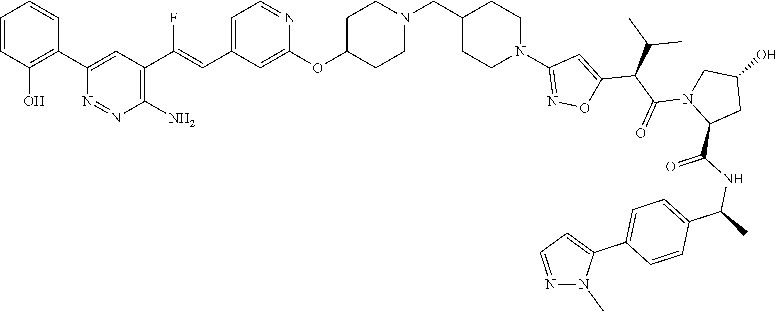 [SMILES: CC(C)[C@@H](C(=O)N1C[C@H](O)C[C@H]1C(=O)N[C@@H](C)c1ccc(-c2ccnn2C)cc1)c1cc(N2CCC(CN3CCC(Oc4cc(/C=C(\F)c5cc(-c6ccccc6O)nnc5N)ccn4)CC3)CC2)no1] | (2S,4R)-1-[(2R)-2-{3-[4-({4-[(4-{2-[3- amino-6-(2-hydroxyphenyl)pyridazin-4- yl]-2-fluoroethenyl}pyridin-2- yl)oxy]piperidin-1-yl}methyl)piperidin-1- yl]-1,2-oxazol-5-yl}-3-methylbutanoyl]-4- hydroxy-N-[(1S)-1-[4-(1-methyl-1H- pyrazol-5-yl)phenyl]ethyl]pyrrolidine-2- carboxamide |
| 53 | 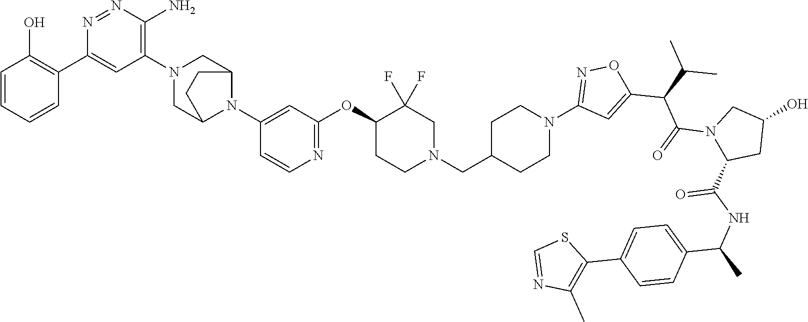 [SMILES: Cc1ncsc1-c1ccc([C@H](C)NC(=O)[C@H]2C[C@@H](O)CN2C(=O)[C@@H](c2cc(N3CCC(CN4CC[C@@H](Oc5cc(N6C7CCC6CN(c6cc(-c8ccccc8O)nnc6N)C7)ccn5)C(F)(F)C4)CC3)no2)C(C)C)cc1] | (2S,4R)-1-[(2R)-2-[3-(4-{[(4R)-4-[(4-{3- [3-amino-6-(2-hydroxyphenyl)pyridazin- 4-yl]-3,8-diazabicyclo[3.2.1]octan-8- yl}pyridin-2-yl)oxy]-3,3- difluoropiperidin-1-yl]methyl}piperidin-1- yl)-1,2-oxazol-5-yl]-3-methylbutanoyl]-4- hydroxy-N-[(1S)-1-[4-(4-methyl-1,3- thiazol-5-yl)phenyl]ethyl]pyrrolidine-2- carboxamide |
| 54 | 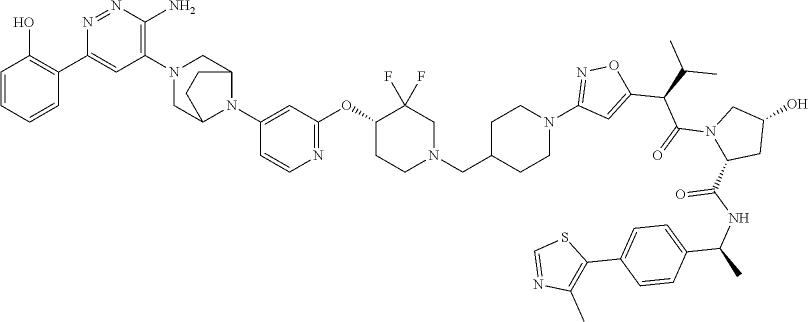 [SMILES: Cc1ncsc1-c1ccc([C@H](C)NC(=O)[C@H]2C[C@@H](O)CN2C(=O)[C@@H](c2cc(N3CCC(CN4CC[C@H](Oc5cc(N6C7CCC6CN(c6cc(-c8ccccc8O)nnc6N)C7)ccn5)C(F)(F)C4)CC3)no2)C(C)C)cc1] | (2S,4R)-1-[(2R)-2-[3-(4-{[(4S)-4-[(4-{3- [3-amino-6-(2-hydroxyphenyl)pyridazin- 4-yl]-3,8-diazabicyclo[3.2.1]octan-8- yl}pyridin-2-yl)oxy]-3,3- difluoropiperidin-1-yl]methyl}piperidin-1- yl)-1,2-oxazol-5-yl]-3-methylbutanoyl]-4- hydroxy-N-[(1S)-1-[4-(4-methyl-1,3- thiazol-5-yl)phenyl]ethyl]pyrrolidine-2- carboxamide |
| 55 | 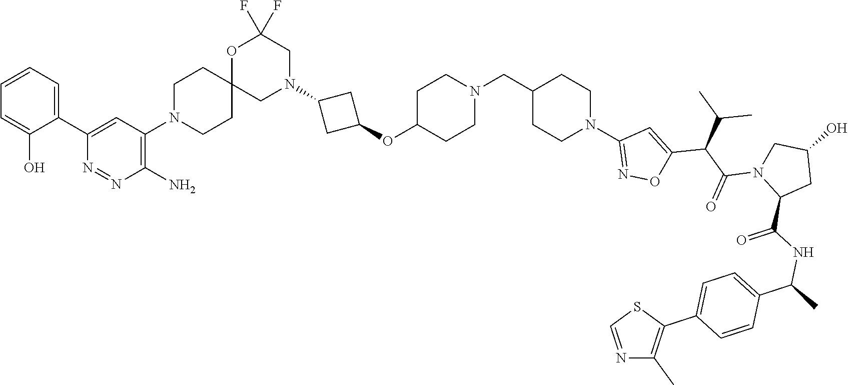 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CC(F)(F)OC7(CCN(c8cc(-c9ccccc9O)nnc8N)CC7)C6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3-{9- [3-amino-6-(2-hydroxyphenyl)pyridazin- 4-yl]-2,2-difluoro-1-oxa-4,9- diazaspiro[5.5]undecan-4- yl}cyclobutoxy]piperidin-1- yl}methyl)piperidin-1-yl]-1,2-oxazol-5- yl}butanoyl]pyrrolidine-2-carboxamide |
| 56 | 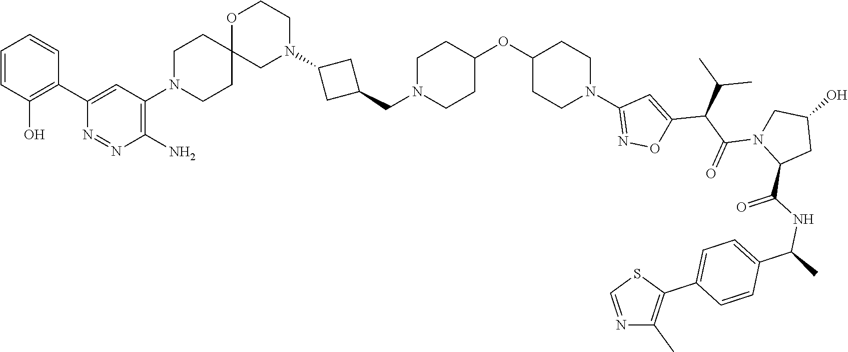 [SMILES: Cc1ncsc1-c1ccc([C@H](C)NC(=O)[C@@H]2C[C@@H](O)CN2C(=O)[C@@H](c2cc(N3CCC(OC4CCN(C[C@H]5C[C@H](N6CCOC7(CCN(c8cc(-c9ccccc9O)nnc8N)CC7)C6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-(3-{4-[(1-{[(1r,3r)-3-{9- [3-amino-6-(2-hydroxyphenyl)pyridazin- 4-yl]-1-oxa-4,9-diazaspiro[5.5]undecan-4- yl}cyclobutyl]methyl}piperidin-4- yl)oxy]piperidin-1-yl}-1,2-oxazol-5- yl)butanoyl]pyrrolidine-2-carboxamide |
| 57 | 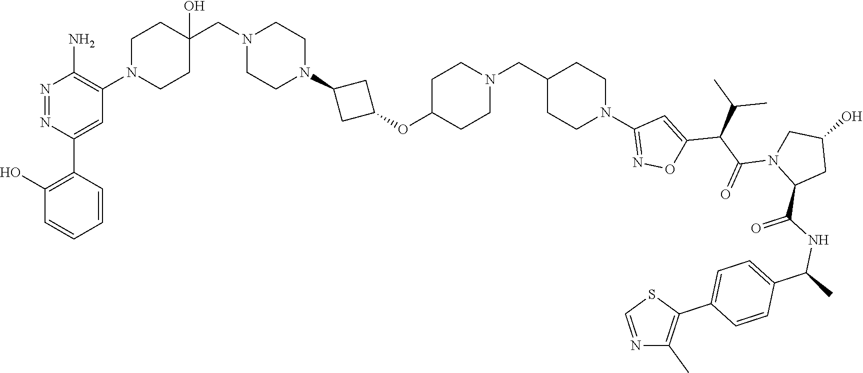 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCN(CC7(O)CCN(c8cc(-c9ccccc9O)nnc8N)CC7)CC6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3-[4- ({1-[3-amino-6-(2- hydroxyphenyl)pyridazin-4-yl]-4- hydroxypiperidin-4-yl}methyl)piperazin- 1-yl]cyclobutoxy]piperidin-1- yl}methyl)piperidin-1-yl]-1,2-oxazol-5- yl}butanoyl]pyrrolidine-2-carboxamide |
| 58 | 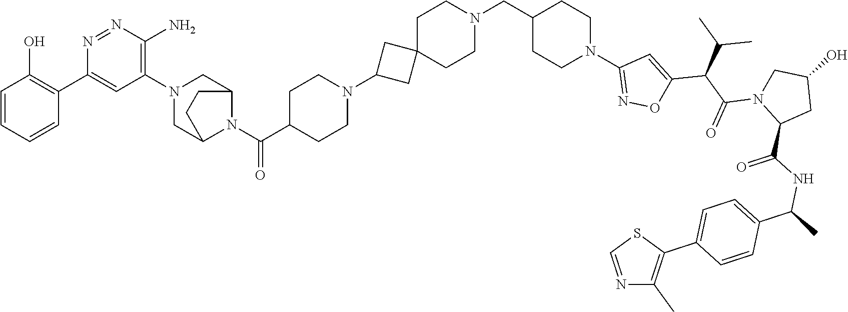 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(N4CCC(C(=O)N6C7CCC6CN(c6cc(-c8ccccc8O)nnc6N)C7)CC4)C5)CC3)no2)C(C)C)cc1] | (2S,4R)-1-[(2R)-2-[3-(4-{[2-(4-{3-[3- amino-6-(2-hydroxyphenyl)pyridazin-4- yl]-3,8-diazabicyclo[3.2.1]octane-8- carbonyl}piperidin-1-yl)-7- azaspiro[3.5]nonan-7-yl]methyl}piperidin- 1-yl)-1,2-oxazol-5-yl]-3-methylbutanoyl]- 4-hydroxy-N-[(1S)-1-[4-(4-methyl-1,3- thiazol-5-yl)phenyl]ethyl]pyrrolidine-2- carboxamide |
| 59 | 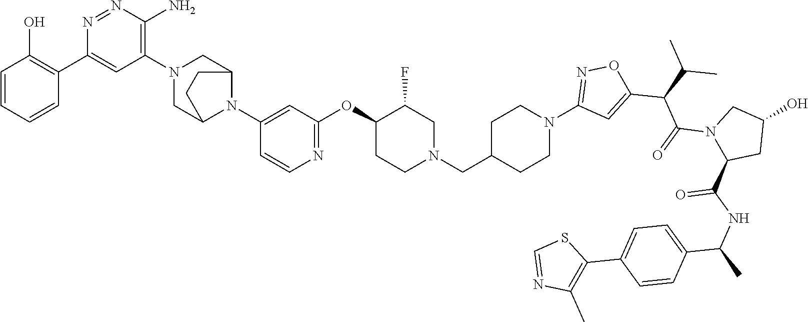 [SMILES: Cc1ncsc1-c1ccc([C@H](C)NC(=O)[C@@H]2C[C@@H](O)CN2C(=O)[C@@H](c2cc(N3CCC(CN4CC[C@@H](Oc5cc(N6C7CCC6CN(c6cc(-c8ccccc8O)nnc6N)C7)ccn5)[C@H](F)C4)CC3)no2)C(C)C)cc1] | (2S,4R)-1-[(2R)-2-[3-(4-{[(3R,4R)-4-[(4- {3-[3-amino-6-(2- hydroxyphenyl)pyridazin-4-yl]-3,8- diazabicyclo[3.2.1]octan-8-yl}pyridin-2- yl)oxy]-3-fluoropiperidin-1- yl]methyl}piperidin-1-yl)-1,2-oxazol-5- yl]-3-methylbutanoyl]-4-hydroxy-N-[(1S)- 1-[4-(4-methyl-1,3-thiazol-5- yl)phenyl]ethyl]pyrrolidine-2- carboxamide |
| 60 | 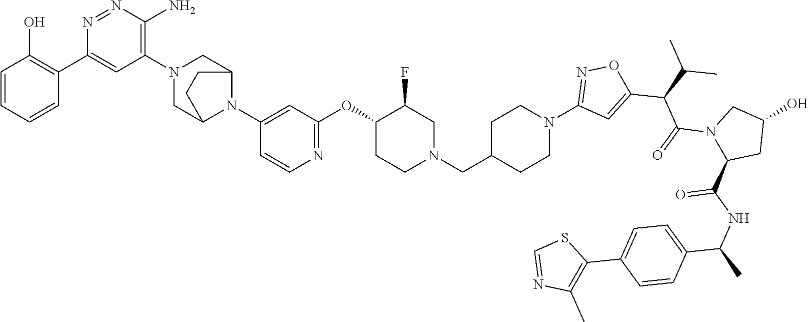 [SMILES: Cc1ncsc1-c1ccc([C@H](C)NC(=O)[C@@H]2C[C@@H](O)CN2C(=O)[C@@H](c2cc(N3CCC(CN4CC[C@H](Oc5cc(N6C7CCC6CN(c6cc(-c8ccccc8O)nnc6N)C7)ccn5)[C@@H](F)C4)CC3)no2)C(C)C)cc1] | (2S,4R)-1-[(2R)-2-[3-(4-{[(3S,4S)-4-[(4- {3-[3-amino-6-(2- hydroxyphenyl)pyridazin-4-yl]-3,8- diazabicyclo[3.2.1]octan-8-yl}pyridin-2- yl)oxy]-3-fluoropiperidin-1- yl]methyl}piperidin-1-yl)-1,2-oxazol-5- yl]-3-methylbutanoyl]-4-hydroxy-N-[(1S)- 1-[4-(4-methyl-1,3-thiazol-5- yl)phenyl]ethyl]pyrrolidine-2- carboxamide |
| 61 | 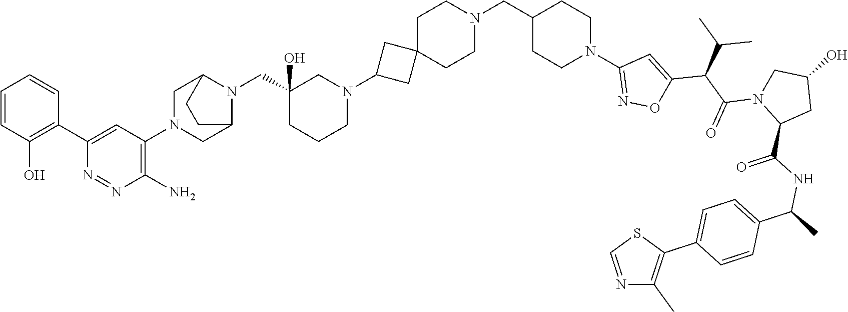 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(N4CCC[C@](O)(CN6C7CCC6CN(c6cc(-c8ccccc8O)nnc6N)C7)C4)C5)CC3)no2)C(C)C)cc1] | (2S,4R)-1-[(2R)-2-{3-[4-({2-[(3R)-3-({3- [3-amino-6-(2-hydroxyphenyl)pyridazin- 4-yl]-3,8-diazabicyclo[3.2.1]octan-8- yl}methyl)-3-hydroxypiperidin-1-yl]-7- azaspiro[3.5]nonan-7-yl}methyl)piperidin- 1-yl]-1,2-oxazol-5-yl}-3-methylbutanoyl]- 4-hydroxy-N-[(1S)-1-[4-(4-methyl-1,3- thiazol-5-yl)phenyl]ethyl]pyrrolidine-2- carboxamide |
| 62 | 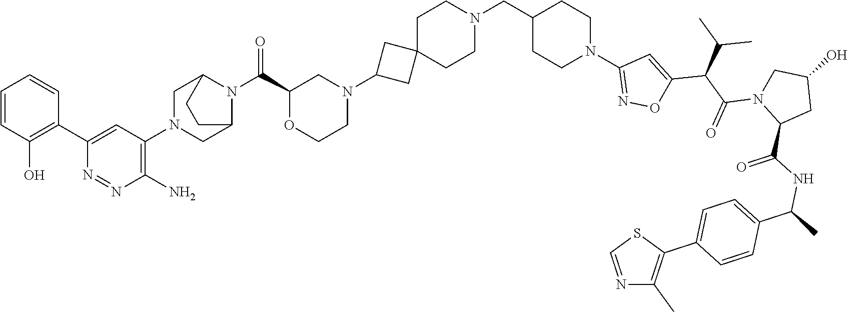 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(N4CCO[C@@H](C(=O)N6C7CCC6CN(c6cc(-c8ccccc8O)nnc6N)C7)C4)C5)CC3)no2)C(C)C)cc1] | (2S,4R)-1-[(2R)-2-{3-[4-({2-[(2R)-2-{3- [3-amino-6-(2-hydroxyphenyl)pyridazin- 4-yl]-3,8-diazabicyclo[3.2.1]octane-8- carbonyl}morpholin-4-yl]-7- azaspiro[3.5]nonan-7-yl}methyl)piperidin- 1-yl]-1,2-oxazol-5-yl}-3-methylbutanoyl]- 4-hydroxy-N-[(1S)-1-[4-(4-methyl-1,3- thiazol-5-yl)phenyl]ethyl]pyrrolidine-2- carboxamide |
| 63 | 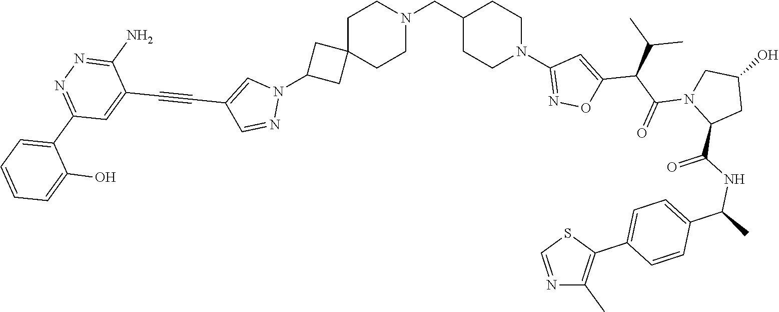 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(n4cc(C#Cc6cc(-c7ccccc7O)nnc6N)cn4)C5)CC3)no2)C(C)C)cc1] | (2S,4R)-1-[(2R)-2-[3-(4-{[2-(4-{2-[3- amino-6-(2-hydroxyphenyl)pyridazin-4- yl]ethynyl}-1H-pyrazol-1-yl)-7- azaspiro[3.5]nonan-7-yl]methyl}piperidin- 1-yl)-1,2-oxazol-5-yl]-3-methylbutanoyl]- 4-hydroxy-N-[(1S)-1-[4-(4-methyl-1,3- thiazol-5-yl)phenyl]ethyl]pyrrolidine-2- carboxamide |
| 64 | 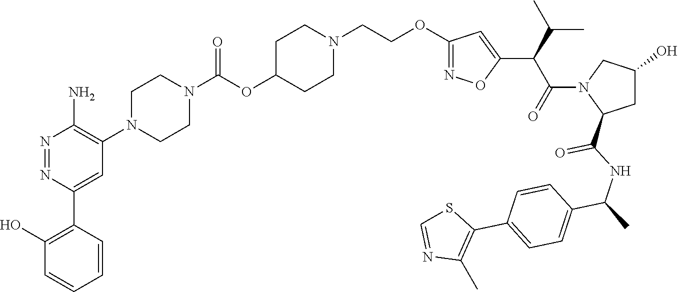 [SMILES: Cc1ncsc1-c1ccc([C@H](C)NC(=O)[C@@H]2C[C@@H](O)CN2C(=O)[C@@H](c2cc(OCCN3CCC(OC(=O)N4CCN(c5cc(-c6ccccc6O)nnc5N)CC4)CC3)no2)C(C)C)cc1] | 1-[2-({5-[(2R)-1-[(2S,4R)-4-hydroxy-2- {[(1S)-1-[4-(4-methyl-1,3-thiazol-5- yl)phenyl]ethyl]carbamoyl}pyrrolidin-1- yl]-3-methyl-1-oxobutan-2-yl]-1,2-oxazol- 3-yl}oxy)ethyl]piperidin-4-yl 4-[3-amino- 6-(2-hydroxyphenyl)pyridazin-4- yl]piperazine-1-carboxylate |
| 65 | 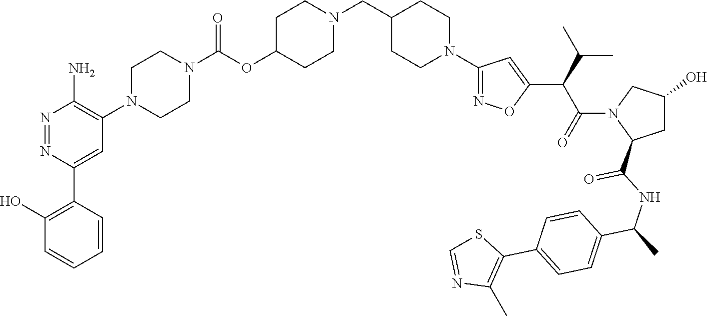 [SMILES: Cc1ncsc1-c1ccc([C@H](C)NC(=O)[C@@H]2C[C@@H](O)CN2C(=O)[C@@H](c2cc(N3CCC(CN4CCC(OC(=O)N5CCN(c6cc(-c7ccccc7O)nnc6N)CC5)CC4)CC3)no2)C(C)C)cc1] | 1-[(1-{5-[(2R)-1-[(2S,4R)-4-hydroxy-2- {[(1S)-1-[4-(4-methyl-1,3-thiazol-5- yl)phenyl]ethyl]carbamoyl}pyrrolidin-1- yl]-3-methyl-1-oxobutan-2-yl]-1,2-oxazol- 3-yl}piperidin-4-yl)methyl]piperidin-4-yl 4-[3-amino-6-(2- hydroxyphenyl)pyridazin-4-yl]piperazine- 1-carboxylate |
| 66 | 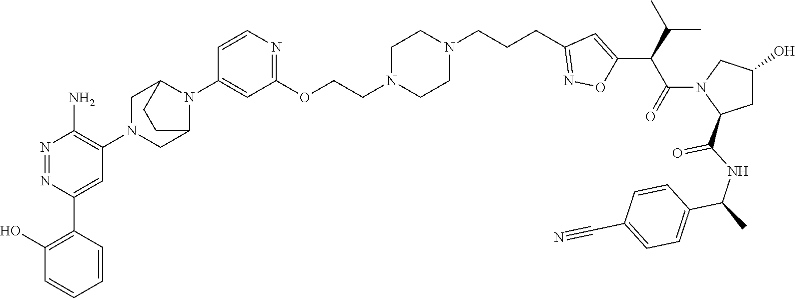 [SMILES: CC(C)[C@@H](C(=O)N1C[C@H](O)C[C@H]1C(=O)N[C@@H](C)c1ccc(C#N)cc1)c1cc(CCCN2CCN(CCOc3cc(N4C5CCC4CN(c4cc(-c6ccccc6O)nnc4N)C5)ccn3)CC2)no1] | (2S,4R)-1-[(2R)-2-{3-[2-(4-{2-[(4-{3-[3- amino-6-(2-hydroxyphenyl)pyridazin-4- yl]-3,8-diazabicyclo[3.2.1]octan-8- yl}pyridin-2-yl)oxy]ethyl}piperazin-1- yl)ethoxy]-1,2-oxazol-5-yl}-3- methylbutanoyl]-N-[(1S)-1-(4- cyanophenyl)ethyl]-4-hydroxypyrrolidine- 2-carboxamide |
| 67 | 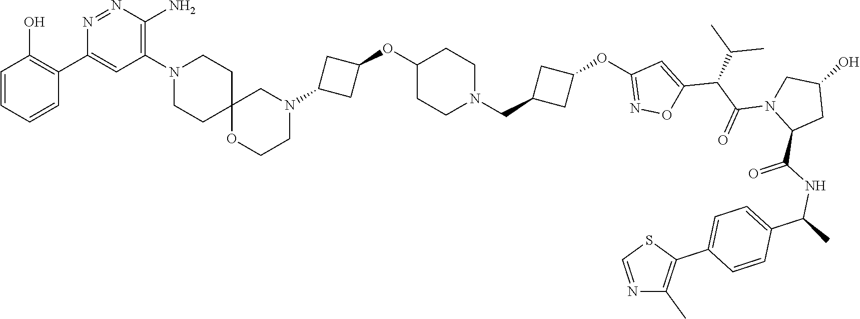 [SMILES: Cc1ncsc1-c1ccc([C@H](C)NC(=O)[C@@H]2C[C@@H](O)CN2C(=O)[C@H](c2cc(O[C@H]3C[C@H](CN4CCC(O[C@H]5C[C@H](N6CCOC7(CCN(c8cc(-c9ccccc9O)nnc8N)CC7)C6)C5)CC4)C3)no2)C(C)C)cc1] | (2S,4R)-4-hydroxy-N-[(1S)-1-[4-(4- methyl-1,3-thiazol-5-yl)phenyl]ethyl]-1- [(2S)-3-methyl-2-{3-[(1r,3r)-3-({4- [(1r,3r)-3-{9-[3-amino-6-(2- hydroxyphenyl)pyridazin-4-yl]-1-oxa-4,9- diazaspiro[5.5]undecan-4- yl}cyclobutoxy]piperidin-1- yl}methyl)cyclobutoxy]-1,2-oxazol-5- yl}butanoyl]pyrrolidine-2-carboxamide |
| 68 | 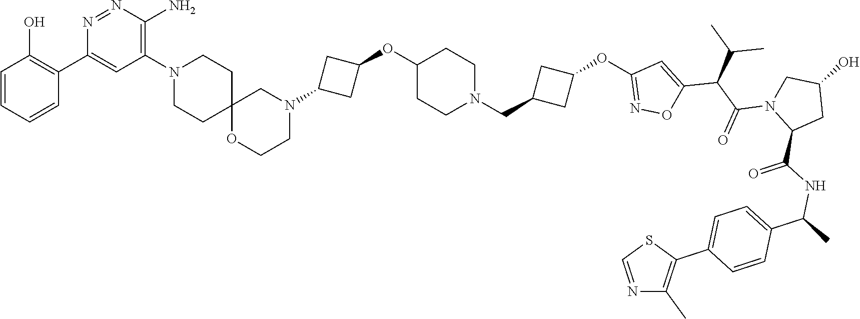 [SMILES: Cc1ncsc1-c1ccc([C@H](C)NC(=O)[C@@H]2C[C@@H](O)CN2C(=O)[C@@H](c2cc(O[C@H]3C[C@H](CN4CCC(O[C@H]5C[C@H](N6CCOC7(CCN(c8cc(-c9ccccc9O)nnc8N)CC7)C6)C5)CC4)C3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[(1r,3r)-3-({4- [(1r,3r)-3-{9-[3-amino-6-(2- hydroxyphenyl)pyridazin-4-yl]-1-oxa-4,9- diazaspiro[5.5]undecan-4- yl}cyclobutoxy]piperidin-1- yl}methyl)cyclobutoxy]-1,2-oxazol-5- yl}butanoyl]pyrrolidine-2-carboxamide |
| 69 | 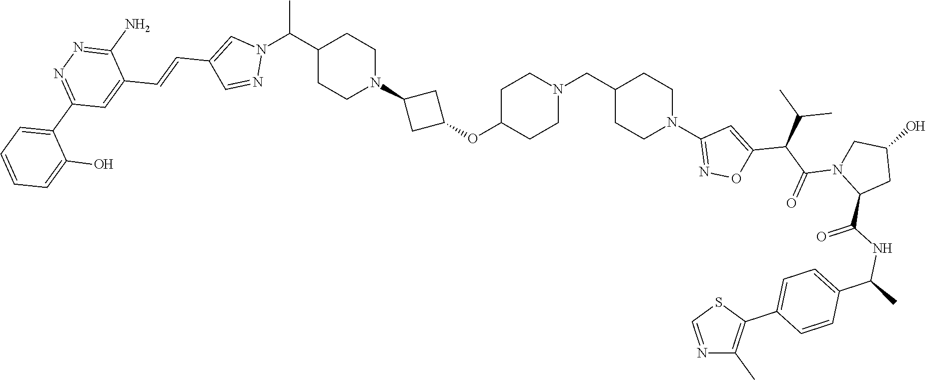 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCC(C(C)n7cc(/C=C/c8cc(-c9ccccc9O)nnc8N)cn7)CC6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3-[4- (1-{4-[(1E)-2-[3-amino-6-(2- hydroxyphenyl)pyridazin-4-yl]ethenyl]- 1H-pyrazol-1-yl}ethyl)piperidin-1- yl]cyclobutoxy]piperidin-1- yl}methyl)piperidin-1-yl]-1,2-oxazol-5- yl}butanoyl]pyrrolidine-2-carboxamide |
| 70 | 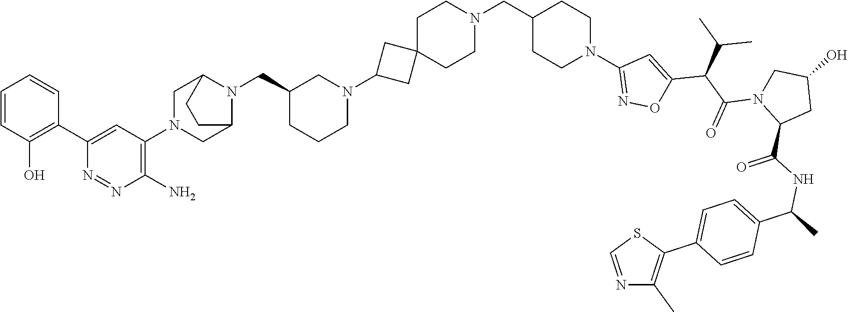 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(N4CCC[C@@H](CN6C7CCC6CN(c6cc(-c8ccccc8O)nnc6N)C7)C4)C5)CC3)no2)C(C)C)cc1] | (2S,4R)-1-[(2R)-2-{3-[4-({2-[(3R)-3-({3- [3-amino-6-(2-hydroxyphenyl)pyridazin- 4-yl]-3,8-diazabicyclo[3.2.1]octan-8- yl}methyl)piperidin-1-yl]-7- azaspiro[3.5]nonan-7-yl}methyl)piperidin- 1-yl]-1,2-oxazol-5-yl}-3-methylbutanoyl]- 4-hydroxy-N-[(1S)-1-[4-(4-methyl-1,3- thiazol-5-yl)phenyl]ethyl]pyrrolidine-2- carboxamide |
| 71 | 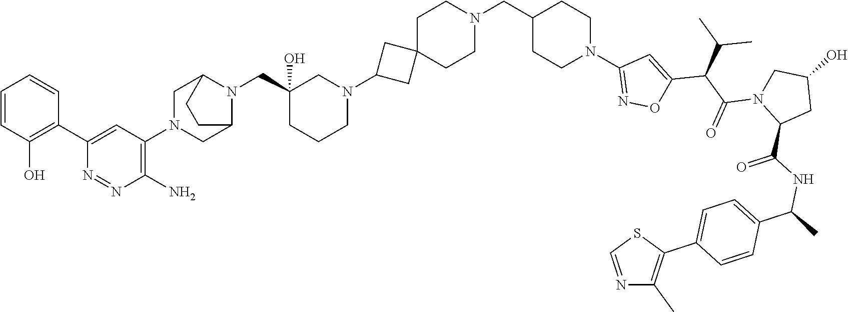 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(N4CCC[C@@](O)(CN6C7CCC6CN(c6cc(-c8ccccc8O)nnc6N)C7)C4)C5)CC3)no2)C(C)C)cc1] | (2S,4R)-1-[(2R)-2-{3-[4-({2-[(3S)-3-({3- [3-amino-6-(2-hydroxyphenyl)pyridazin- 4-yl]-3,8-diazabicyclo[3.2.1]octan-8- yl}methyl)-3-hydroxypiperidin-1-yl]-7- azaspiro[3.5]nonan-7-yl}methyl)piperidin- 1-yl]-1,2-oxazol-5-yl}-3-methylbutanoyl]- 4-hydroxy-N-[(1S)-1-[4-(4-methyl-1,3- thiazol-5-yl)phenyl]ethyl]pyrrolidine-2- carboxamide |
| 72 | 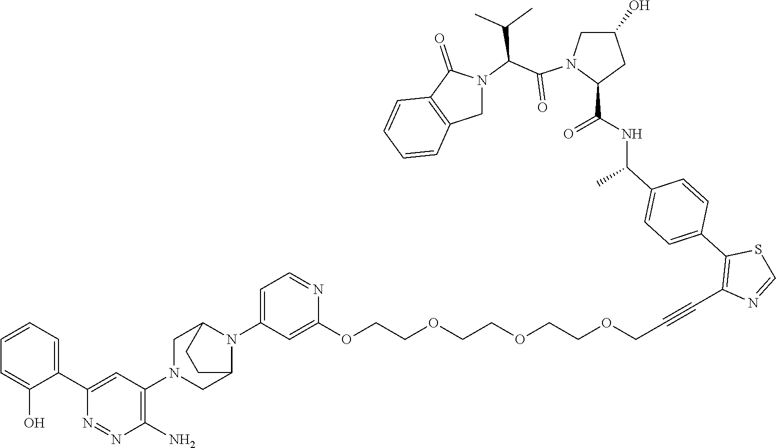 [SMILES: CC(C)[C@@H](C(=O)N1C[C@H](O)C[C@H]1C(=O)N[C@@H](C)c1ccc(-c2scnc2C#CCOCCOCCOCCOc2cc(N3C4CCC3CN(c3cc(-c5ccccc5O)nnc3N)C4)ccn2)cc1)N1Cc2ccccc2C1=O] | (2S,4R)-N-[(1S)-1-[4-(4-{3-[2-(2-{2-[(4- {3-[3-amino-6-(2- hydroxyphenyl)pyridazin-4-yl]-3,8- diazabicyclo[3.2.1]octan-8-yl}pyridin-2- yl)oxy]ethoxy}ethoxy)ethoxy]prop-1-yn- 1-yl}-1,3-thiazol-5-yl)phenyl]ethyl]-4- hydroxy-1-[(2S)-3-methyl-2-(1-oxo-2,3- dihydro-1H-isoindol-2- yl)butanoyl]pyrrolidine-2-carboxamide |
| 73 | 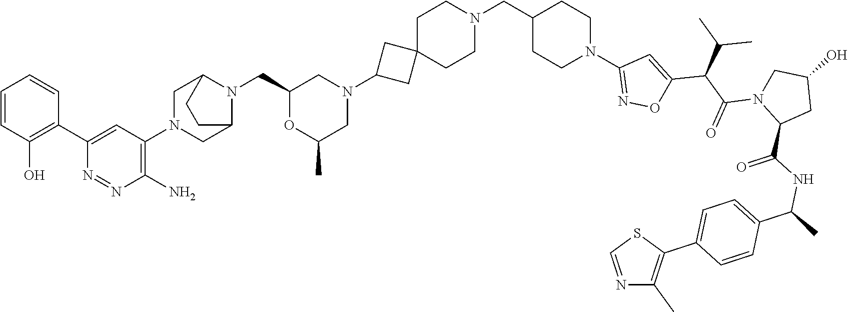 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(N4C[C@H](CN6C7CCC6CN(c6cc(-c8ccccc8O)nnc6N)C7)O[C@H](C)C4)C5)CC3)no2)C(C)C)cc1] | (2S,4R)-1-[(2R)-2-{3-[4-({2-[(2R,6R)-2- ({3-[3-amino-6-(2- hydroxyphenyl)pyridazin-4-yl]-3,8- diazabicyclo[3.2.1]octan-8-yl}methyl)-6- methylmorpholin-4-yl]-7- azaspiro[3.5]nonan-7-yl}methyl)piperidin- 1-yl]-1,2-oxazol-5-yl}-3-methylbutanoyl]- 4-hydroxy-N-[(1S)-1-[4-(4-methyl-1,3- thiazol-5-yl)phenyl]ethyl]pyrrolidine-2- carboxamide |
| 74 | 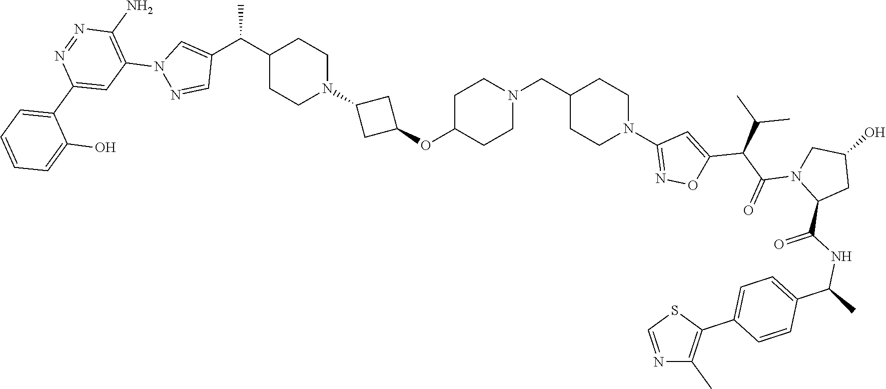 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCC([C@@H](C)c7cnn(-c8cc(-c9ccccc9O)nnc8N)c7)CC6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3-{4- [(1R)-1-{1-[3-amino-6-(2- hydroxyphenyl)pyridazin-4-yl]-1H- pyrazol-4-yl}ethyl]piperidin-- yl}cyclobutoxy]piperidin-1- yl}methyl)piperidin-1-yl]-1,2-oxazol-5- yl}butanoyl]pyrrolidine-2-carboxamide |
| 75 | 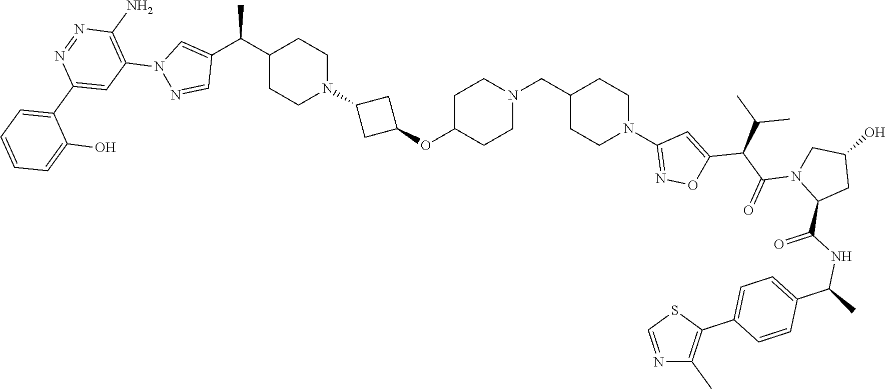 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCC([C@H](C)c7cnn(-c8cc(-c9ccccc9O)nnc8N)c7)CC6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3-{4- [(1S)-1-{1-[3-amino-6-(2- hydroxyphenyl)pyridazin-4-yl]-1H- pyrazol-4-yl}ethyl]piperidin-1- yl}cyclobutoxy]piperidin-1- yl}methyl)piperidin-1-yl]-1,2-oxazol-5- yl}butanoyl]pyrrolidine-2-carboxamide |
| 76 | 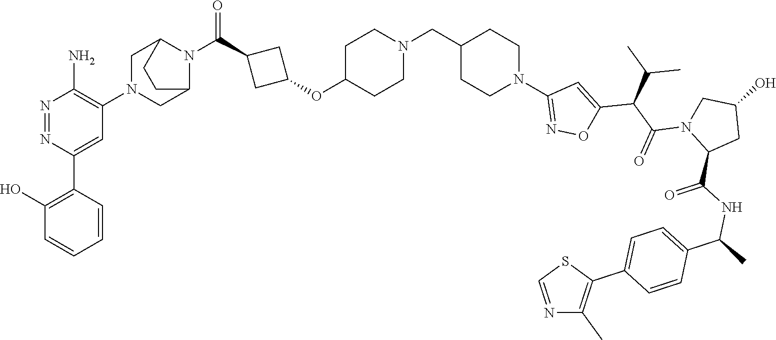 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](C(=O)N6C7CCC6CN(c6cc(-c8ccccc8O)nnc6N)C7)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3-(4- {3-[3-amino-6-(2- hydroxyphenyl)pyridazin-4-yl]-3,8- diazabicyclo[3.2.1]octan-8-yl}piperidine- 1-carbonyl)cyclobutoxy]piperidin-1- yl}methyl)piperidin-1-yl]-1,2-oxazol-5- yl}butanoyl]pyrrolidine-2-carboxamide |
| 77 | 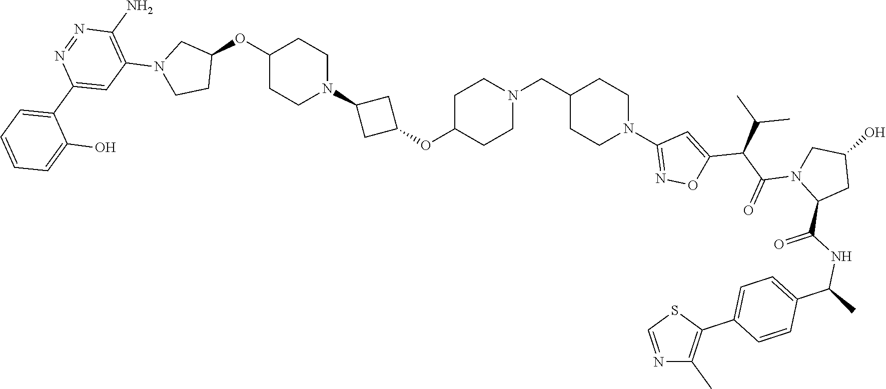 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCC(O[C@H]7CCN(c8cc(-c9ccccc9O)nnc8N)C7)CC6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3-(4- {[(3S)-1-[3-amino-6-(2- hydroxyphenyl)pyridazin-4-yl]pyrrolidin- 3-yl]oxy}piperidin-1- yl)cyclobutoxy]piperidin-1- yl}methyl)piperidin-1-yl]-1,2-oxazol-5- yl}butanoyl]pyrrolidine-2-carboxamide |
| 78 | 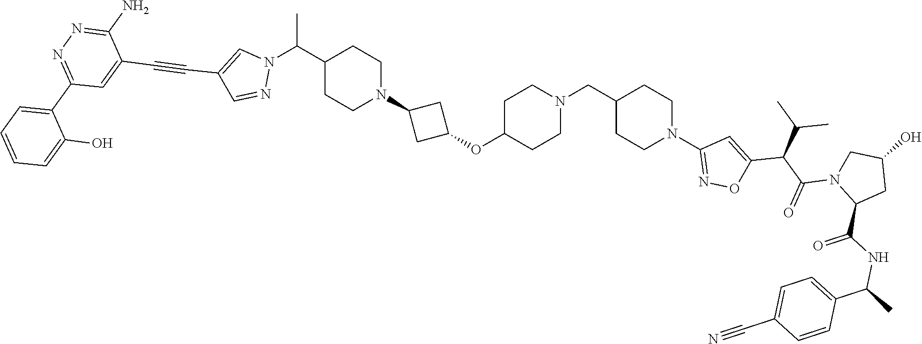 [SMILES: CC(C)[C@@H](C(=O)N1C[C@H](O)C[C@H]1C(=O)N[C@@H](C)c1ccc(C#N)cc1)c1cc(N2CCC(CN3CCC(O[C@H]4C[C@H](N5CCC(C(C)n6cc(C#Cc7cc(-c8ccccc8O)nnc7N)cn6)CC5)C4)CC3)CC2)no1] | (2S,4R)-N-[(1S)-1-(4-cyanophenyl)ethyl]- 4-hydroxy-1-[(2R)-3-methyl-2-{3-[4-({4- [(1r,3r)-3-{4-[1-(4-{2-[3-amino-6-(2- hydroxyphenyl)pyridazin-4-yl]ethynyl}- 1H-pyrazol-1-yl)ethyl]piperidin-1- yl}cyclobutoxy]piperidin-1- yl}methyl)piperidin-1-yl]-1,2-oxazol-5- yl}butanoyl]pyrrolidine-2-carboxamide |
| 79 | 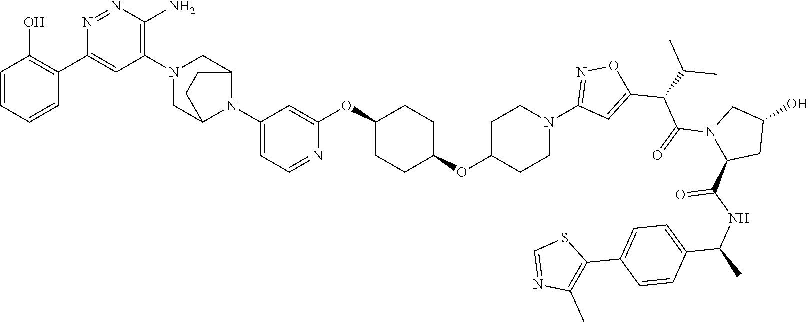 [SMILES: Cc1ncsc1-c1ccc([C@H](C)NC(=O)[C@@H]2C[C@@H](O)CN2C(=O)[C@H](c2cc(N3CCC(O[C@H]4CC[C@@H](Oc5cc(N6C7CCC6CN(c6cc(-c8ccccc8O)nnc6N)C7)ccn5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2S)-3-methyl-2-[3-(4-{[(1s,4s)-4-[(4-{3- [3-amino-6-(2-hydroxyphenyl)pyridazin- 4-yl]-3,8-diazabicyclo[3.2.1]octan-8- yl}pyridin-2- yl)oxy]cyclohexyl]oxy}piperidin-1-yl)- 1,2-oxazol-5-yl]butanoyl]pyrrolidine-2- carboxamide |
| 80 | 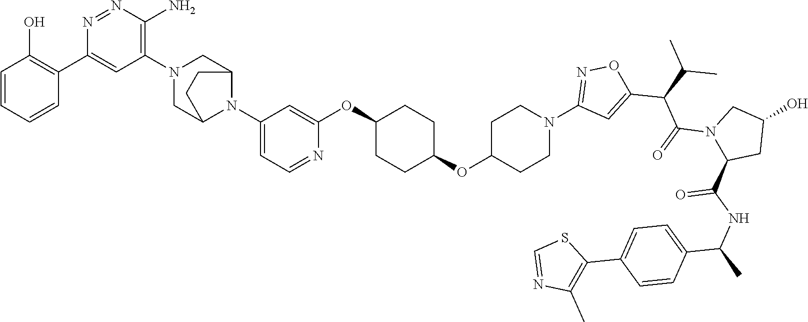 [SMILES: Cc1ncsc1-c1ccc([C@H](C)NC(=O)[C@@H]2C[C@@H](O)CN2C(=O)[C@@H](c2cc(N3CCC(O[C@H]4CC[C@@H](Oc5cc(N6C7CCC6CN(c6cc(-c8ccccc8O)nnc6N)C7)ccn5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-[3-(4-{[(1s,4s)-4-[(4-{3- [3-amino-6-(2-hydroxyphenyl)pyridazin- 4-yl]-3,8-diazabicyclo[3.2.1]octan-8- yl}pyridin-2- yl)oxy]cyclohexyl]oxy}piperidin-1-yl)- 1,2-oxazol-5-yl]butanoyl]pyrrolidine-2- carboxamide |
| 81 | 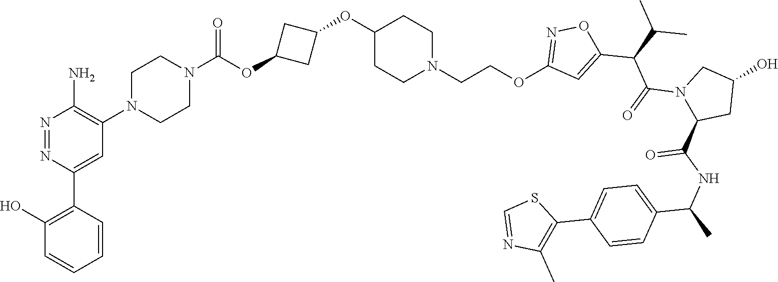 [SMILES: Cc1ncsc1-c1ccc([C@H](C)NC(=O)[C@@H]2C[C@@H](O)CN2C(=O)[C@@H](c2cc(OCCN3CCC(O[C@H]4C[C@H](OC(=O)N5CCN(c6cc(-c7ccccc7O)nnc6N)CC5)C4)CC3)no2)C(C)C)cc1] | (1r,3r)-3-({1-[2-({5-[(2R)-1-[(2S,4R)-4- hydroxy-2-{[(1S)-1-[4-(4-methyl-1,3- thiazol-5- yl)phenyl]ethyl]carbamoyl}pyrrolidin-1- yl]-3-methyl-1-oxobutan-2-yl]-1,2-oxazol- 3-yl}oxy)ethyl]piperidin-4- yl}oxy)cyclobutyl 4-[3-amino-6-(2- hydroxyphenyl)pyridazin-4-yl]piperazine- 1-carboxylate |
| 82 | 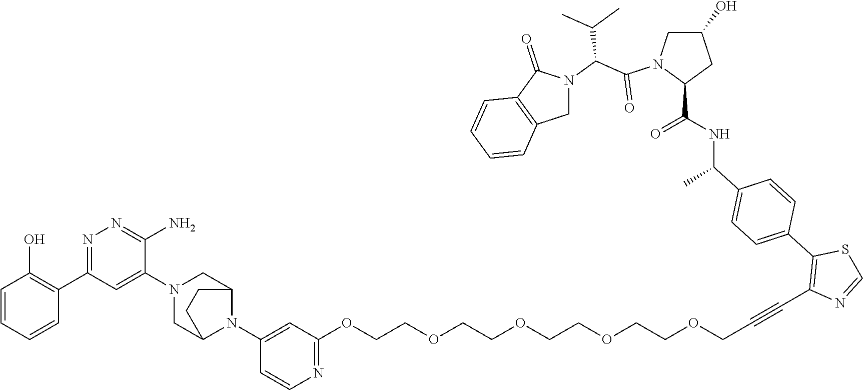 [SMILES: CC(C)[C@H](C(=O)N1C[C@H](O)C[C@H]1C(=O)N[C@@H](C)c1ccc(-c2scnc2C#CCOCCOCCOCCOCCOc2cc(N3C4CCC3CN(c3cc(-c5ccccc5O)nnc3N)C4)ccn2)cc1)N1Cc2ccccc2C1=O] | (2S,4R)-N-[(1S)-1-[4-(4-{1-[(4-{3-[3- amino-6-(2-hydroxyphenyl)pyridazin-4- yl]-3,8-diazabicyclo[3.2.1]octan-8- yl}pyridin-2-yl)oxy]-3,6,9,12- tetraoxapentadec-14-yn-15-yl}-1,3- thiazol-5-yl)phenyl]ethyl]-4-hydroxy-1- [(2R)-3-methyl-2-(1-oxo-2,3-dihydro-1H- isoindol-2-yl)butanoyl]pyrrolidine-2- carboxamide |
| 83 | 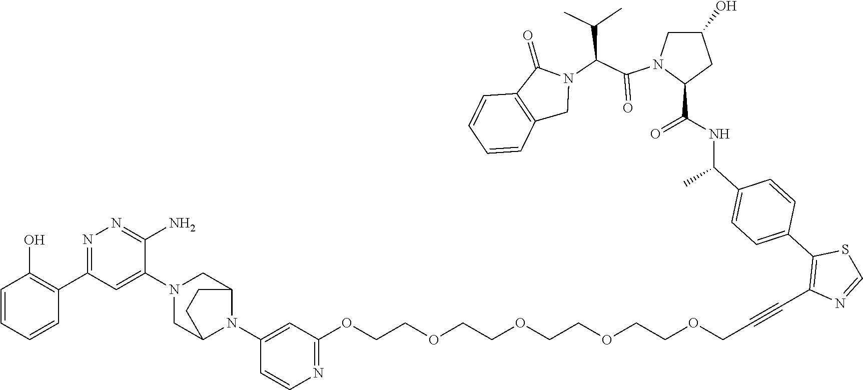 [SMILES: CC(C)[C@@H](C(=O)N1C[C@H](O)C[C@H]1C(=O)N[C@@H](C)c1ccc(-c2scnc2C#CCOCCOCCOCCOCCOc2cc(N3C4CCC3CN(c3cc(-c5ccccc5O)nnc3N)C4)ccn2)cc1)N1Cc2ccccc2C1=O] | (2S,4R)-N-[(1S)-1-[4-(4-{1-[(4-{3-[3- amino-6-(2-hydroxyphenyl)pyridazin-4- yl]-3,8-diazabicyclo[3.2.1]octan-8- yl}pyridin-2-yl)oxy]-3,6,9,12- tetraoxapentadec-14-yn-15-yl}-1,3- thiazol-5-yl)phenyl]ethyl]-4-hydroxy-1- [(2S)-3-methyl-2-(1-oxo-2,3-dihydro-1H- isoindol-2-yl)butanoyl]pyrrolidine-2- carboxamide |
| 84 | 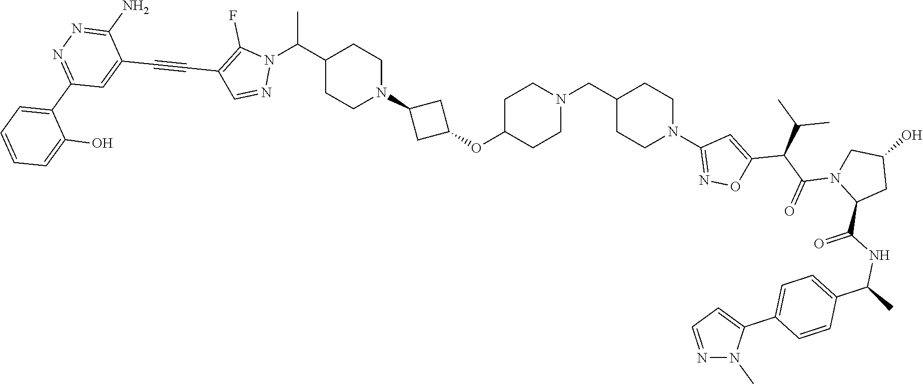 [SMILES: CC(C)[C@@H](C(=O)N1C[C@H](O)C[C@H]1C(=O)N[C@@H](C)c1ccc(-c2ccnn2C)cc1)c1cc(N2CCC(CN3CCC(O[C@H]4C[C@H](N5CCC(C(C)n6ncc(C#Cc7cc(-c8ccccc8O)nnc7N)c6F)CC5)C4)CC3)CC2)no1] | (2S,4R)-4-hydroxy-N-[(1S)-1-[4-(1- methyl-1H-pyrazol-5-yl)phenyl]ethyl]-1- [(2R)-3-methyl-2-{3-[4-({4-[(1r,3r)-3-{4- [1-(4-{2-[3-amino-6-(2- hydroxyphenyl)pyridazin-4-yl]ethynyl}-5- fluoro-1H-pyrazol-1-yl)ethyl]piperidin-1- yl}cyclobutoxy]piperidin-1- yl}methyl)piperidin-1-yl]-1,2-oxazol-5- yl}butanoyl]pyrrolidine-2-carboxamide |
| 85 | 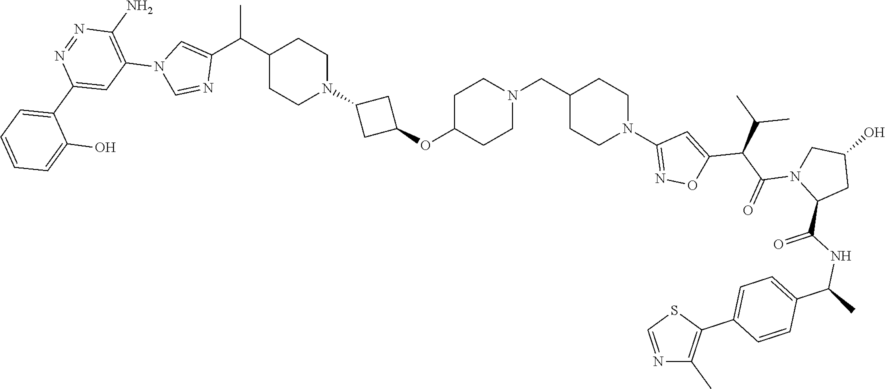 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCC(C(C)c7cn(-c8cc(-c9ccccc9O)nnc8N)cn7)CC6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3-[4- (1-{1-[3-amino-6-(2- hydroxyphenyl)pyridazin-4-yl]-1H- imidazol-4-yl}ethyl)piperidin-1- yl]cyclobutoxy]piperidin-1- yl}methyl)piperidin-1-yl]-1,2-oxazol-5- yl}butanoyl]pyrrolidine-2-carboxamide |
| 86 | 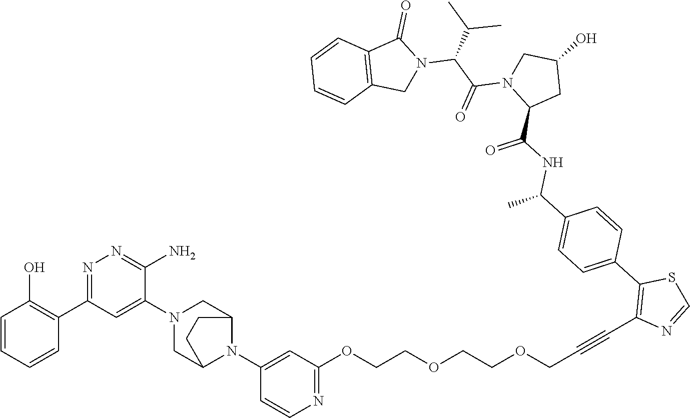 [SMILES: CC(C)[C@H](C(=O)N1C[C@H](O)C[C@H]1C(=O)N[C@@H](C)c1ccc(-c2scnc2C#CCOCCOCCOc2cc(N3C4CCC3CN(c3cc(-c5ccccc5O)nnc3N)C4)ccn2)cc1)N1Cc2ccccc2C1=O] | (2S,4R)-N-[(1S)-1-(4-{4-[3-(2-{2-[(4-{3- [3-amino-6-(2-hydroxyphenyl)pyridazin- 4-yl]-3,8-diazabicyclo[3.2.1]octan-8- yl}pyridin-2-yl)oxyJethoxy}ethoxy)prop- 1-yn-1-yl]-1,3-thiazol-5-yl}phenyl)ethyl]- 4-hydroxy-1-[(2R)-3-methyl-2-(1-oxo-2,3- dihydro-1H-isoindol-2- yl)butanoyl]pyrrolidine-2-carboxamide |
| 87 | 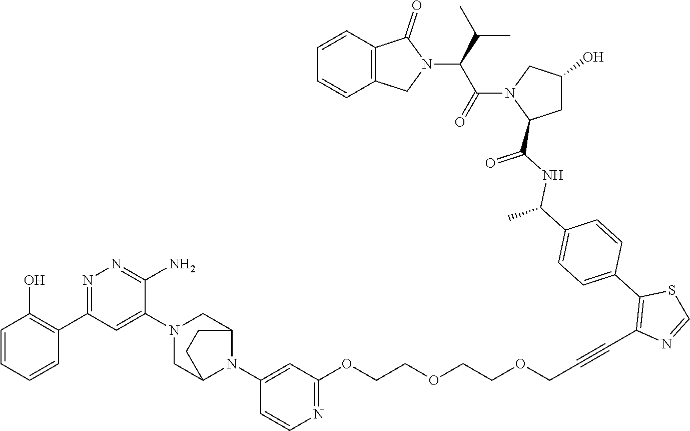 [SMILES: CC(C)[C@@H](C(=O)N1C[C@H](O)C[C@H]1C(=O)N[C@@H](C)c1ccc(-c2scnc2C#CCOCCOCCOc2cc(N3C4CCC3CN(c3cc(-c5ccccc5O)nnc3N)C4)ccn2)cc1)N1Cc2ccccc2C1=O] | (2S,4R)-N-[(1S)-1-(4-{4-[3-(2-{2-[(4-{3- [3-amino-6-(2-hydroxyphenyl)pyridazin- 4-yl]-3,8-diazabicyclo[3.2.1]octan-8- yl}pyridin-2-yl)oxy]ethoxy}ethoxy)prop- 1-yn-1-yl]-1,3-thiazol-5-yl}phenyl)ethyl]- 4-hydroxy-1-[(2S)-3-methyl-2-(1-oxo-2,3- dihydro-1H-isoindol-2- yl)butanoyl]pyrrolidine-2-carboxamide |
| 88 | 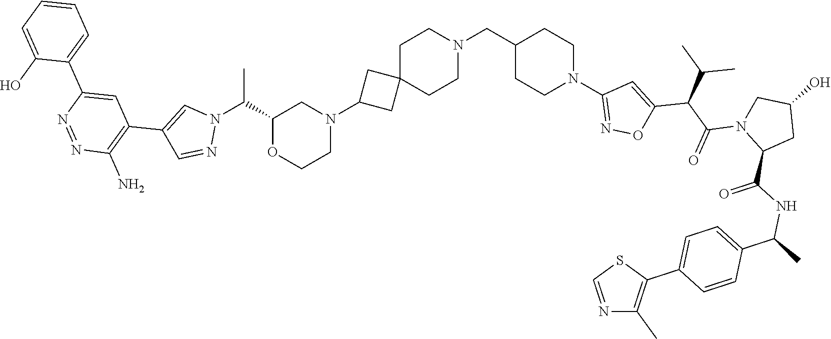 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(N4CCO[C@H](C(C)n6cc(-c7cc(-c8ccccc8O)nnc7N)cn6)C4)C5)CC3)no2)C(C)C)cc1] | (2S,4R)-1-[(2R)-2-{3-[4-({2-[(2S)-2-(1- (4-[3-amino-6-(2- hydroxyphenyl)pyridazin-4-yl]-1H- pyrazol-1-yl}ethyl)morpholin-4-yl]-7- azaspiro[3.5]nonan-7-yl}methyl)piperidin- 1-yl]-1,2-oxazol-5-yl}-3-methylbutanoyl]- 4-hydroxy-N-[(1S)-1-[4-(4-methyl-1,3- thiazol-5-yl)phenyl]ethyl]pyrrolidine-2- carboxamide |
| 89 | 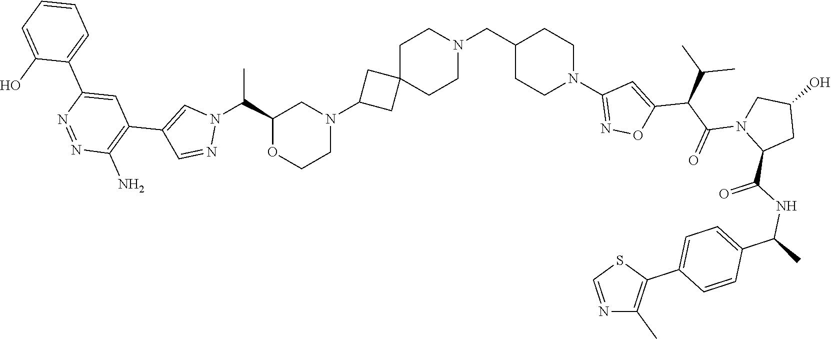 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(N4CCO[C@@H](C(C)n6cc(-c7cc(-c8ccccc8O)nnc7N)cn6)C4)C5)CC3)no2)C(C)C)cc1] | (2S,4R)-1-[(2R)-2-{3-[4-({2-[(2R)-2-(1- {4-[3-amino-6-(2- hydroxyphenyl)pyridazin-4-yl]-1H- pyrazol-1-yl}ethyl)morpholin-4-yl]-7- azaspiro[3.5]nonan-7-yl}methyl)piperidin- 1-yl]-1,2-oxazol-5-yl}-3-methylbutanoyl]- 4-hydroxy-N-[(1S)-1-[4-(4-methyl-1,3- thiazol-5-yl)phenyl]ethyl]pyrrolidine-2- carboxamide |
| 90 | 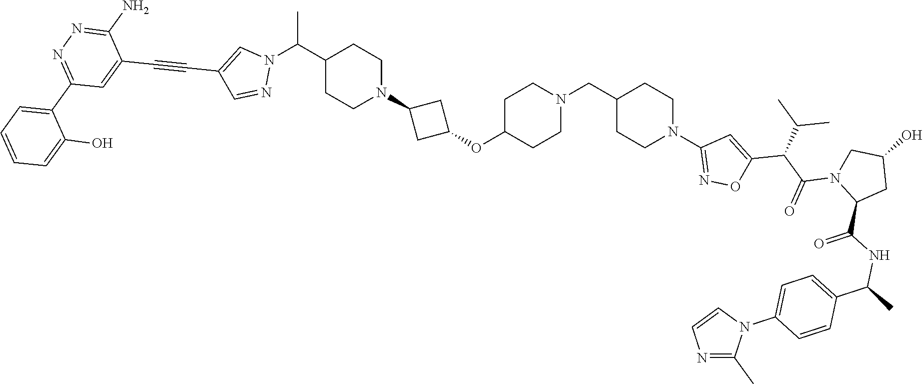 [SMILES: Cc1nccn1-c1ccc([C@H](C)NC(=O)[C@@H]2C[C@@H](O)CN2C(=O)[C@H](c2cc(N3CCC(CN4CCC(O[C@H]5C[C@H](N6CCC(C(C)n7cc(C#Cc8cc(-c9ccccc9O)nnc8N)cn7)CC6)C5)CC4)CC3)no2)C(C)C)cc1] | (2S,4R)-4-hydroxy-N-[(1S)-1-[4-(2- methyl-1H-imidazol-1-yl)phenyl]ethyl]-1- [(2S)-3-methyl-2-{3-[4-({4-[(1r,3r)-3-{4- [1-(4-{2-[3-amino-6-(2- hydroxyphenyl)pyridazin-4-yl]ethynyl}- 1H-pyrazol-1-yl)ethyl]piperidin-1- yl}cyclobutoxy]piperidin-1- yl}methyl)piperidin-1-yl]-1,2-oxazol-5- yl}butanoyl]pyrrolidine-2-carboxamide |
| 91 | 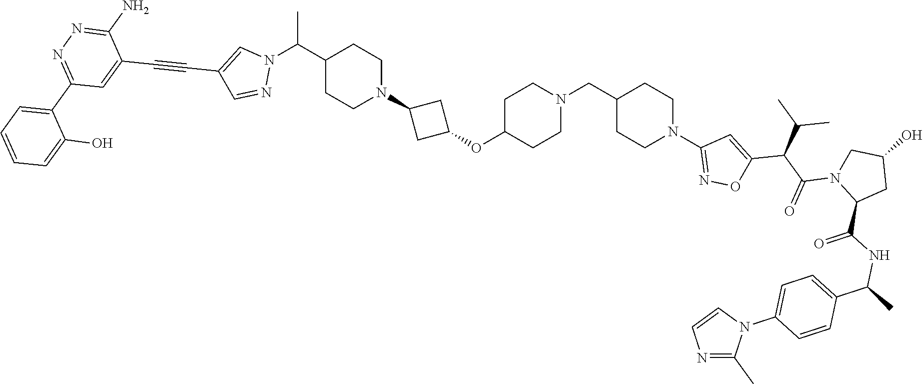 [SMILES: Cc1nccn1-c1ccc([C@H](C)NC(=O)[C@@H]2C[C@@H](O)CN2C(=O)[C@@H](c2cc(N3CCC(CN4CCC(O[C@H]5C[C@H](N6CCC(C(C)n7cc(C#Cc8cc(-c9ccccc9O)nnc8N)cn7)CC6)C5)CC4)CC3)no2)C(C)C)cc1] | (2S,4R)-4-hydroxy-N-[(1S)-1-[4-(2- methyl-1H-imidazol-1-yl)phenyl]ethyl]-1- [(2R)-3-methyl-2-{3-[4-({4-[(1r,3r)-3-{4- [1-(4-{2-[3-amino-6-(2- hydroxyphenyl)pyridazin-4-yl]ethynyl}- 1H-pyrazol-1-yl)ethyl]piperidin-1- yl}cyclobutoxy]piperidin-1- yl}methyl)piperidin-1-yl]-1,2-oxazol-5- yl}butanoyl]pyrrolidine-2-carboxamide |
| 92 | 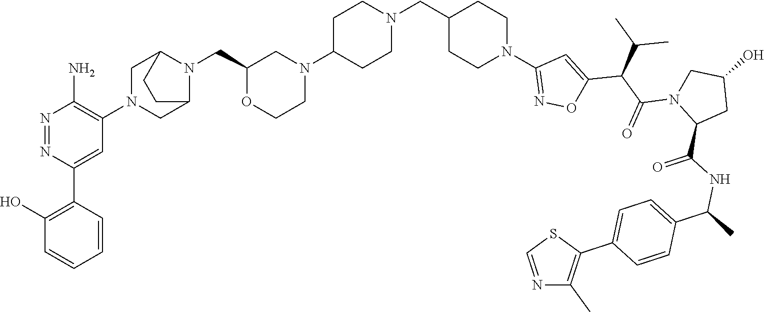 [SMILES: Cc1ncsc1-c1ccc([C@H](C)NC(=O)[C@@H]2C[C@@H](O)CN2C(=O)[C@@H](c2cc(N3CCC(CN4CCC(N5CCO[C@@H](CN6C7CCC6CN(c6cc(-c8ccccc8O)nnc6N)C7)C5)CC4)CC3)no2)C(C)C)cc1] | (2S,4R)-1-[(2R)-2-{3-[4-({4-[(2R)-2-({3- [3-amino-6-(2-hydroxyphenyl)pyridazin- 4-yl]-3,8-diazabicyclo[3.2.1]octan-8- yl}methyl)morpholin-4-yl]piperidin-1- yl}methyl)piperidin-1-yl]-1,2-oxazol-5- yl}-3-methylbutanoyl]-4-hydroxy-N- [(1S)-1-[4-(4-methyl-1,3-thiazol-5- yl)phenyl]ethyl]pyrrolidine-2- carboxamide |
| 93 | 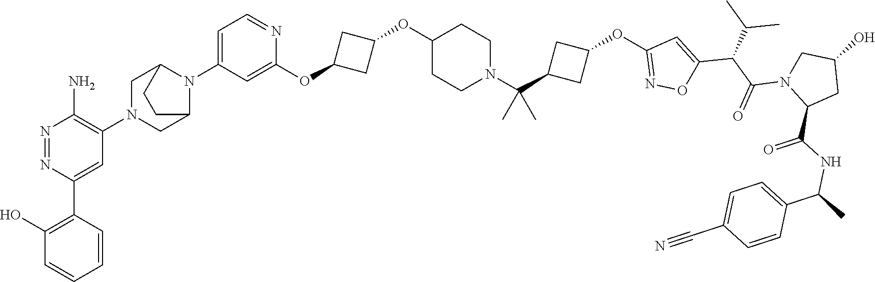 [SMILES: CC(C)[C@H](C(=O)N1C[C@H](O)C[C@H]1C(=O)N[C@@H](C)c1ccc(C#N)cc1)c1cc(O[C@H]2C[C@H](C(C)(C)N3CCC(O[C@H]4C[C@H](Oc5cc(N6C7CCC6CN(c6cc(-c8ccccc8O)nnc6N)C7)ccn5)C4)CC3)C2)no1] | (2S,4R)-N-[(1S)-1-(4-cyanophenyl)ethyl]- 4-hydroxy-1-[(2S)-3-methyl-2-{3-[(1r,3r)- 3-(2-{4-[(1r,3r)-3-[(4-{3-[3-amino-6-(2- hydroxyphenyl)pyridazin-4-yl]-3,8- diazabicyclo[3.2.1]octan-8-yl}pyridin-2- yl)oxy]cyclobutoxy]piperidin-1- yl}propan-2-yl)cyclobutoxy]-1,2-oxazol- 5-yl}butanoyl]pyrrolidine-2-carboxamide |
| 94 | 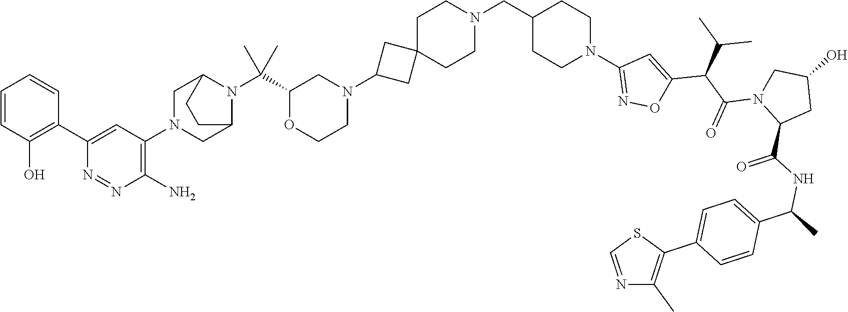 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(N4CCO[C@H](C(C)(C)N6C7CCC6CN(c6cc(-c8ccccc8O)nnc6N)C7)C4)C5)CC3)no2)C(C)C)cc1] | (2S,4R)-1-[(2R)-2-{3-[4-({2-[(2S)-2-(2- {3-[3-amino-6-(2- hydroxyphenyl)pyridazin-4-yl]-3,8- diazabicyclo[3.2.1]octan-8-yl}propan-2- yl)morpholin-4-yl]-7-azaspiro[3.5]nonan- 7-yl}methyl)piperidin-1-yl]-1,2-oxazol-5- yl}-3-methylbutanoyl]-4-hydroxy-N- [(1S)-1-[4-(4-methyl-1,3-thiazol-5- yl)phenyl]ethyl]pyrrolidine-2- carboxamide |
| 95 | 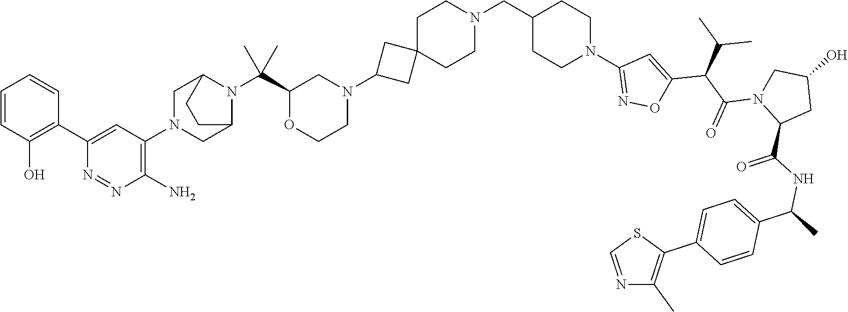 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(N4CCO[C@@H](C(C)(C)N6C7CCC6CN(c6cc(-c8ccccc8O)nnc6N)C7)C4)C5)CC3)no2)C(C)C)cc1] | (2S,4R)-1-[(2R)-2-{3-[4-({2-[(2R)-2-(2- {3-[3-amino-6-(2- hydroxyphenyl)pyridazin-4-yl]-3,8- diazabicyclo[3.2.1]octan-8-yl}propan-2- yl)morpholin-4-yl]-7-azaspiro[3.5]nonan- 7-yl}methyl)piperidin-1-yl]-1,2-oxazol-5- yl}-3-methylbutanoyl]-4-hydroxy-N- [(1S)-1-[4-(4-methyl-1,3-thiazol-5- yl)phenyl]ethyl]pyrrolidine-2- carboxamide |
| 96 | 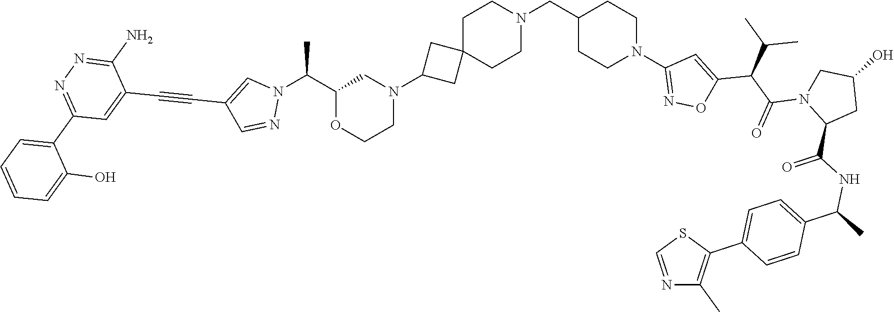 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(N4CCO[C@H]([C@H](C)n6cc(C#Cc7cc(-c8ccccc8O)nnc7N)cn6)C4)C5)CC3)no2)C(C)C)cc1] | (2S,4R)-1-[(2R)-2-{3-[4-({2-[(2S)-2- [(1S)-1-(4-{2-[3-amino-6-(2- hydroxyphenyl)pyridazin-4-yl]ethynyl}- 1H-pyrazol-1-yl)ethyl]morpholin-4-yl]-7- azaspiro[3.5]nonan-7-yl}methyl)piperidin- 1-yl]-1,2-oxazol-5-yl}-3-methylbutanoyl]- 4-hydroxy-N-[(1S)-1-[4-(4-methyl-1,3- thiazol-5-yl)phenyl]ethyl]pyrrolidine-2- carboxamide |
| 97 | 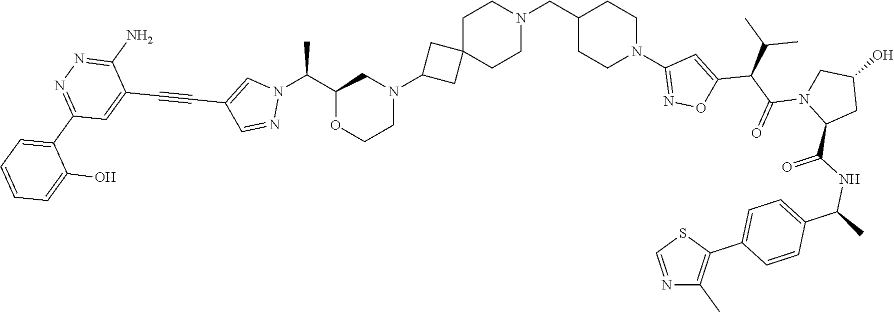 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(N4CCO[C@@H]([C@H](C)n6cc(C#Cc7cc(-c8ccccc8O)nnc7N)cn6)C4)C5)CC3)no2)C(C)C)cc1] | (2S,4R)-1-[(2R)-2-{3-[4-({2-[(2R)-2- [(1S)-1-(4-{2-[3-amino-6-(2- hydroxyphenyl)pyridazin-4-yl]ethynyl}- 1H-pyrazol-1-yl)ethyl]morpholin-4-yl]-7- azaspiro[3.5]nonan-7-yl}methyl)piperidin- 1-yl]-1,2-oxazol-5-yl}-3-methylbutanoyl]- 4-hydroxy-N-[(1S)-1-[4-(4-methyl-1,3- thiazol-5-yl)phenyl]ethyl]pyrrolidine-2- carboxamide |
| 98 | 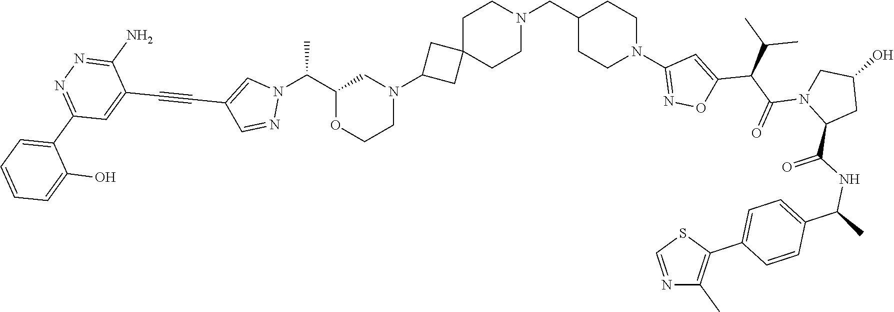 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(N4CCO[C@H]([C@@H](C)n6cc(C#Cc7cc(-c8ccccc8O)nnc7N)cn6)C4)C5)CC3)no2)C(C)C)cc1] | (2S,4R)-1-[(2R)-2-{3-[4-({2-[(2S)-2- [(1R)-1-(4-{2-[3-amino-6-(2- hydroxyphenyl)pyridazin-4-yl]ethynyl}- 1H-pyrazol-1-yl)ethyl]morpholin-4-yl]-7- azaspiro[3.5]nonan-7-yl}methyl)piperidin- 1-yl]-1,2-oxazol-5-yl}-3-methylbutanoyl]- 4-hydroxy-N-[(1S)-1-[4-(4-methyl-1,3- thiazol-5-yl)phenyl]ethyl]pyrrolidine-2- carboxamide |
| 99 | 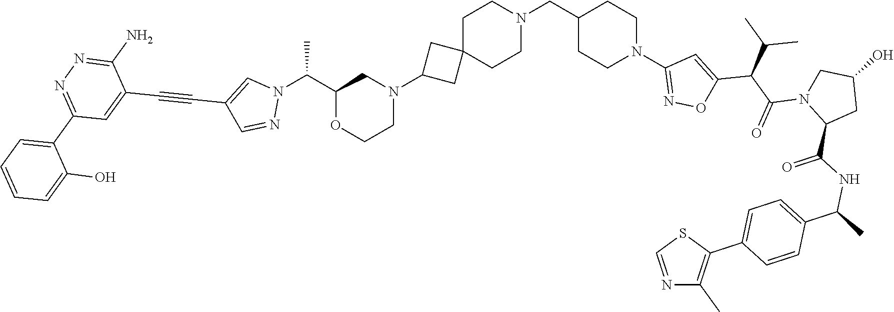 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(N4CCO[C@@H]([C@@H](C)n6cc(C#Cc7cc(-c8ccccc8O)nnc7N)cn6)C4)C5)CC3)no2)C(C)C)cc1] | (2S,4R)-1-[(2R)-2-{3-[4-({2-[(2R)-2- [(1R)-1-(4-{2-[3-amino-6-(2- hydroxyphenyl)pyridazin-4-yl]ethynyl}- 1H-pyrazol-1-yl)ethyl]morpholin-4-yl]-7- azaspiro[3.5]nonan-7-yl}methyl)piperidin- 1-yl]-1,2-oxazol-5-yl}-3-methylbutanoyl]- 4-hydroxy-N-[(1S)-1-[4-(4-methyl-1,3- thiazol-5-yl)phenyl]ethyl]pyrrolidine-2- carboxamide |
| 100 | 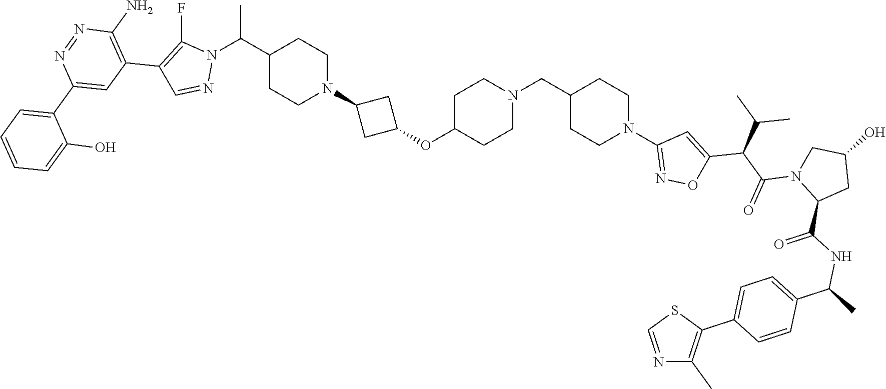 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCC(C(C)n7ncc(-c8cc(-c9ccccc9O)nnc8N)c7F)CC6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3-[4- (1-{4-[3-amino-6-(2- hydroxyphenyl)pyridazin-4-yl]-5-fluoro- 1H-pyrazol-1-yl}ethyl)piperidin-1- yl]cyclobutoxy]piperidin-1- yl}methyl)piperidin-1-yl]-1,2-oxazol-5- yl}butanoyl]pyrrolidine-2-carboxamide |
| 101 | 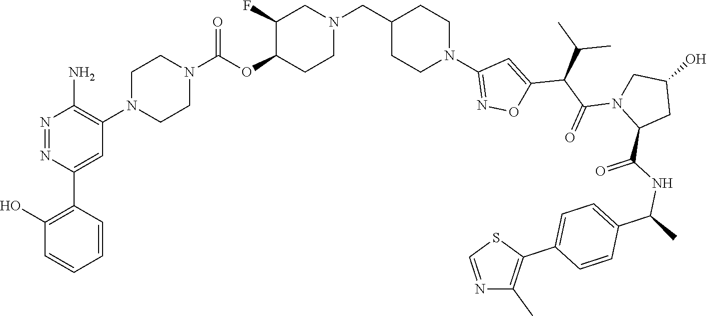 [SMILES: Cc1ncsc1-c1ccc([C@H](C)NC(=O)[C@@H]2C[C@@H](O)CN2C(=O)[C@@H](c2cc(N3CCC(CN4CC[C@@H](OC(=O)N5CCN(c6cc(-c7ccccc7O)nnc6N)CC5)[C@@H](F)C4)CC3)no2)C(C)C)cc1] | (3S,4R)-3-fluoro-1-[(1-{5-[(2R)-1- [(2S,4R)-4-hydroxy-2-{[(1S)-1-[4-(4- methyl-1,3-thiazol-5- yl)phenyl]ethyl]carbamoyl}pyrrolidin-1- yl]-3-methyl-1-oxobutan-2-yl]-1,2-oxazol- 3-yl}piperidin-4-yl)methyl]piperidin-4-yl 4-[3-amino-6-(2- hydroxyphenyl)pyridazin-4-yl]piperazine- 1-carboxylate |
| 102 | 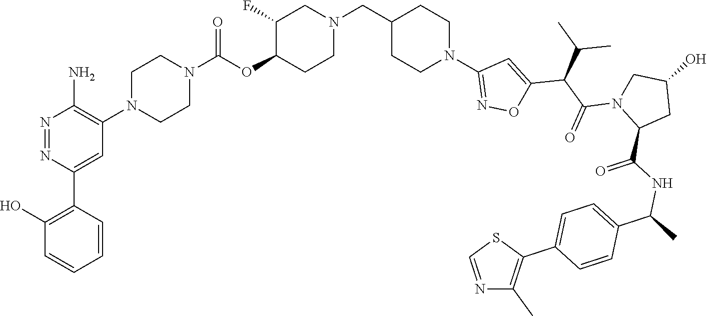 [SMILES: Cc1ncsc1-c1ccc([C@H](C)NC(=O)[C@@H]2C[C@@H](O)CN2C(=O)[C@@H](c2cc(N3CCC(CN4CC[C@@H](OC(=O)N5CCN(c6cc(-c7ccccc7O)nnc6N)CC5)[C@H](F)C4)CC3)no2)C(C)C)cc1] | (3R,4R)-3-fluoro-1-[(1-{5-[(2R)-1- [(2S,4R)-4-hydroxy-2-{[(1S)-1-[4-(4- methyl-1,3-thiazol-5- yl)phenyl]ethyl]carbamoyl}pyrrolidin-1- yl]-3-methyl-1-oxobutan-2-yl]-1,2-oxazol- 3-yl}piperidin-4-yl)methyl]piperidin-4-yl 4-[3-amino-6-(2- hydroxyphenyl)pyridazin-4-yl]piperazine- 1-carboxylate |
| 103 | 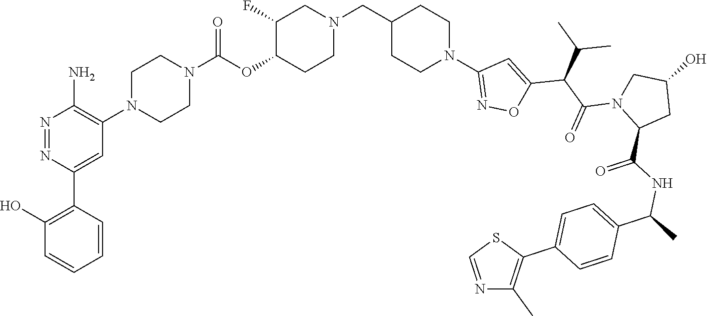 [SMILES: Cc1ncsc1-c1ccc([C@H](C)NC(=O)[C@@H]2C[C@@H](O)CN2C(=O)[C@@H](c2cc(N3CCC(CN4CC[C@H](OC(=O)N5CCN(c6cc(-c7ccccc7O)nnc6N)CC5)[C@H](F)C4)CC3)no2)C(C)C)cc1] | (3R,4S)-3-fluoro-1-[(1-{5-[(2R)-1- [(2S,4R)-4-hydroxy-2-{[(1S)-1-[4-(4- methyl-1,3-thiazol-5- yl)phenyl]ethyl]carbamoyl}pyrrolidin-1- yl]-3-methyl-1-oxobutan-2-yl]-1,2-oxazol- 3-yl}piperidin-4-yl)methyl]piperidin-4-yl 4-[3-amino-6-(2- hydroxyphenyl)pyridazin-4-yl]piperazine- 1-carboxylate |
| 104 | 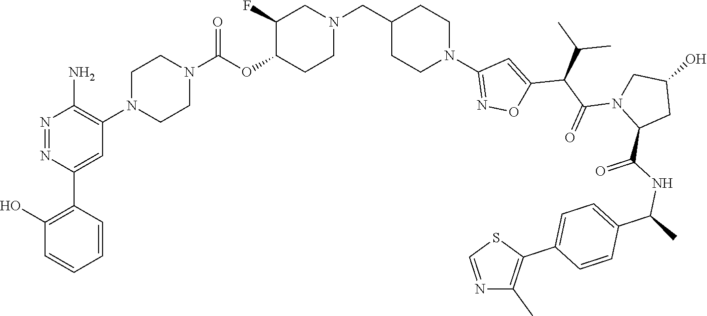 [SMILES: Cc1ncsc1-c1ccc([C@H](C)NC(=O)[C@@H]2C[C@@H](O)CN2C(=O)[C@@H](c2cc(N3CCC(CN4CC[C@H](OC(=O)N5CCN(c6cc(-c7ccccc7O)nnc6N)CC5)[C@@H](F)C4)CC3)no2)C(C)C)cc1] | (3S,4S)-3-fluoro-1-[(1-{5-[(2R)-1- [(2S,4R)-4-hydroxy-2-{[(1S)-1-[4-(4- methyl-1,3-thiazol-5- yl)phenyl]ethyl]carbamoyl}pyrrolidin-1- yl]-3-methyl-1-oxobutan-2-yl]-1,2-oxazol- 3-yl}piperidin-4-yl)methyl]piperidin-4-yl 4-[3-amino-6-(2- hydroxyphenyl)pyridazin-4-yl]piperazine- 1-carboxylate |
| 105 | 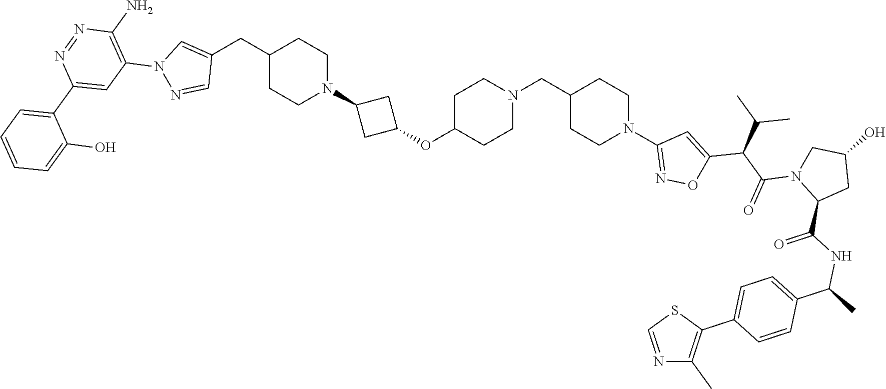 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCC(Cc7cnn(-c8cc(-c9ccccc9O)nnc8N)c7)CC6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3-[4- ({1-[3-amino-6-(2- hydroxyphenyl)pyridazin-4-yl]-1H- pyrazol-4-yl}methyl)piperidin-1- yl]cyclobutoxy]piperidin-1- yl}methyl)piperidin-1-yl]-1,2-oxazol-5- yl}butanoyl]pyrrolidine-2-carboxamide |
| 106 | 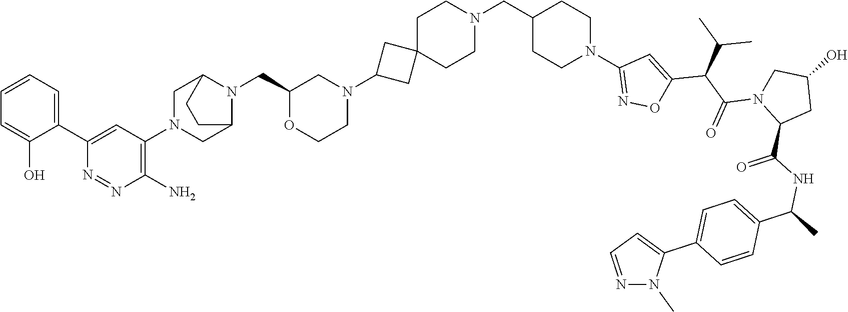 [SMILES: CC(C)[C@@H](C(=O)N1C[C@H](O)C[C@H]1C(=O)N[C@@H](C)c1ccc(-c2ccnn2C)cc1)c1cc(N2CCC(CN3CCC4(CC3)CC(N3CCO[C@@H](CN5C6CCC5CN(c5cc(-c7ccccc7O)nnc5N)C6)C3)C4)CC2)no1] | (2S,4R)-1-[(2R)-2-{3-[4-({2-[(2R)-2-({3- [3-amino-6-(2-hydroxyphenyl)pyridazin- 4-yl]-3,8-diazabicyclo[3.2.1]octan-8- yl}methyl)morpholin-4-yl]-7- azaspiro[3.5]nonan-7-yl}methyl)piperidin- 1-yl]-1,2-oxazol-5-yl}-3-methylbutanoyl]- 4-hydroxy-N-[(1S)-1-[4-(1-methyl-1H- pyrazol-5-yl)phenyl]ethyl]pyrrolidine-2- carboxamide |
| 107 | 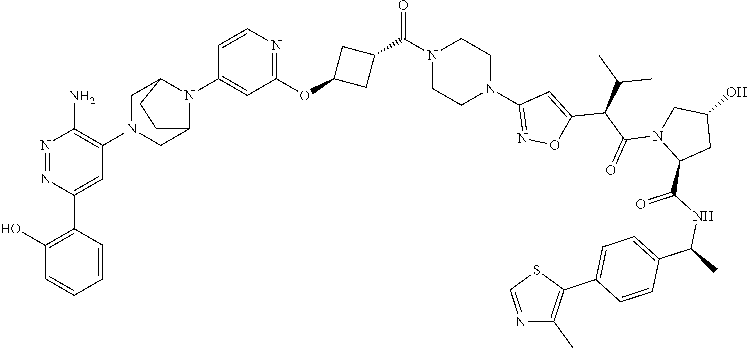 [SMILES: Cc1ncsc1-c1ccc([C@H](C)NC(=O)[C@@H]2C[C@@H](O)CN2C(=O)[C@@H](c2cc(N3CCN(C(=O)[C@H]4C[C@H](Oc5cc(N6C7CCC6CN(c6cc(-c8ccccc8O)nnc6N)C7)ccn5)C4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-(3-{4-[(1r,3r)-3-[(4-{3- [3-amino-6-(2-hydroxyphenyl)pyridazin- 4-yl]-3,8-diazabicyclo[3.2.1]octan-8- yl}pyridin-2- yl)oxy]cyclobutanecarbonyl]piperazin-1- yl}-1,2-oxazol-5-yl)butanoyl]pyrrolidine- 2-carboxamide |
| 108 | 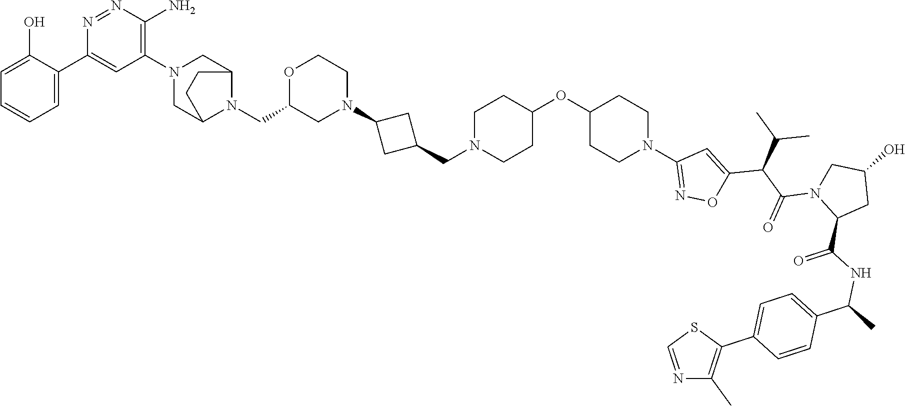 [SMILES: Cc1ncsc1-c1ccc([C@H](C)NC(=O)[C@@H]2C[C@@H](O)CN2C(=O)[C@@H](c2cc(N3CCC(OC4CCN(C[C@H]5C[C@@H](N6CCO[C@@H](CN7C8CCC7CN(c7cc(-c9ccccc9O)nnc7N)C8)C6)C5)CC4)CC3)no2)C(C)C)cc1] | (2S,4R)-1-((2R)-2-(3-(4-((1-(((1S,3s)-3- ((2R)-2-((3-(3-amino-6-(2- hydroxyphenyl)pyridazin-4-yl)-3,8- diazabicyclo[3.2.1]octan-8- yl)methyl)morpholino)cyclobutyl)methyl) piperidin-4-yl)oxy)piperidin-1- yl)isoxazol-5-yl)-3-methylbutanoyl)-4- hydroxy-N-((S)-1-(4-(4-methylthiazol-5- yl)phenyl)ethyl)pyrrolidine-2- carboxamide |
| 109 | 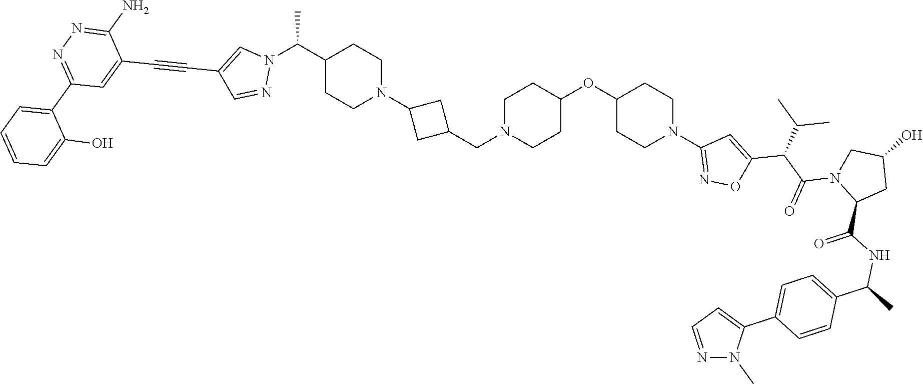 [SMILES: CC(C)[C@H](C(=O)N1C[C@H](O)C[C@H]1C(=O)N[C@@H](C)c1ccc(-c2ccnn2C)cc1)c1cc(N2CCC(OC3CCN(CC4CC(N5CCC([C@@H](C)n6cc(C#Cc7cc(-c8ccccc8O)nnc7N)cn6)CC5)C4)CC3)CC2)no1] | (2S,4R)-1-[(2S)-2-{3-[4-({1-[(3-{4-[(1R)- 1-(4-{2-[3-amino-6-(2- hydroxyphenyl)pyridazin-4-yl]ethynyl}- 1H-pyrazol-1-yl)ethyl]piperidin-1- yl}cyclobutyl)methyl]piperidin-4- yl}oxy)piperidin-1-yl]-1,2-oxazol-5-yl}-3- methylbutanoyl]-4-hydroxy-N-[(1S)-1-[4- (1-methyl-1H-pyrazol-5- yl)phenyl]ethyl]pyrrolidine-2- carboxamide |
| 110 | 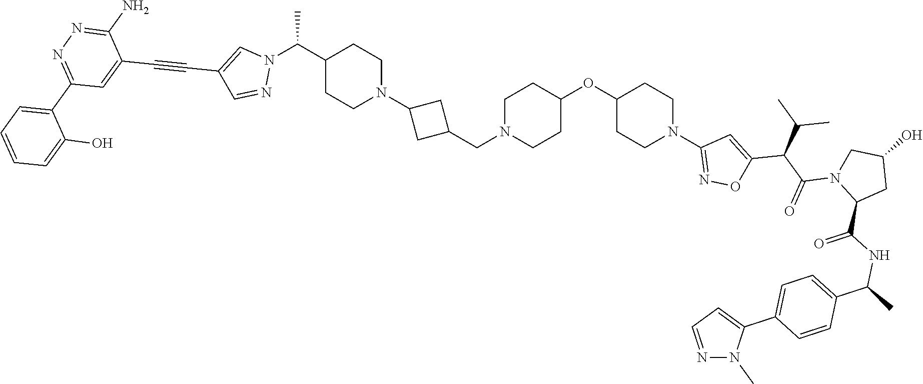 [SMILES: CC(C)[C@@H](C(=O)N1C[C@H](O)C[C@H]1C(=O)N[C@@H](C)c1ccc(-c2ccnn2C)cc1)c1cc(N2CCC(OC3CCN(CC4CC(N5CCC([C@@H](C)n6cc(C#Cc7cc(-c8ccccc8O)nnc7N)cn6)CC5)C4)CC3)CC2)no1] | (2S,4R)-1-[(2R)-2-{3-[4-({1-[(3-{4-[(1R)- 1-(4-{2-[3-amino-6-(2- hydroxyphenyl)pyridazin-4-yl]ethynyl}- 1H-pyrazol-1-yl)ethyl]piperidin-1- yl}cyclobutyl)methyl]piperidin-4- yl}oxy)piperidin-1-yl]-1,2-oxazol-5-yl}-3- methylbutanoyl]-4-hydroxy-N-[(1S)-1-[4- (1-methyl-1H-pyrazol-5- yl)phenyl]ethyl]pyrrolidine-2- carboxamide |
| 111 | 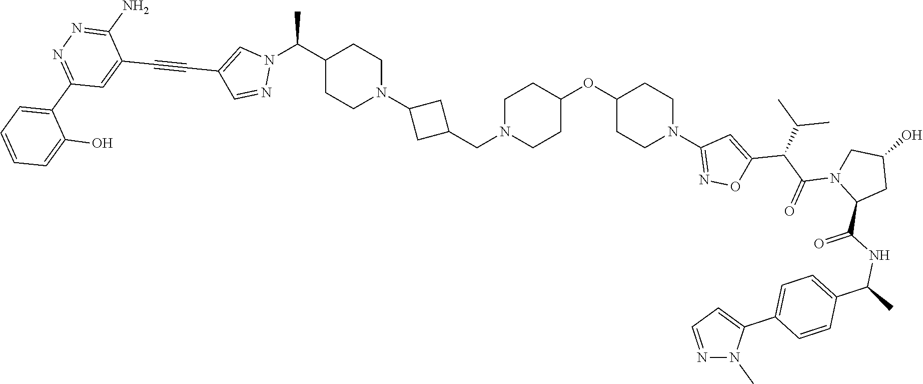 [SMILES: CC(C)[C@H](C(=O)N1C[C@H](O)C[C@H]1C(=O)N[C@@H](C)c1ccc(-c2ccnn2C)cc1)c1cc(N2CCC(OC3CCN(CC4CC(N5CCC([C@H](C)n6cc(C#Cc7cc(-c8ccccc8O)nnc7N)cn6)CC5)C4)CC3)CC2)no1] | (2S,4R)-1-[(2S)-2-{3-[4-({1-[(3-{4-[(1S)- 1-(4-{2-[3-amino-6-(2- hydroxyphenyl)pyridazin-4-yl]ethynyl}- 1H-pyrazol-1-yl)ethyl]piperidin-1- yl}cyclobutyl)methyl]piperidin-4- yl}oxy)piperidin-1-yl]-1,2-oxazol-5-yl}-3- methylbutanoyl]-4-hydroxy-N-[(1S)-1-[4- (1-methyl-1H-pyrazol-5- yl)phenyl]ethyl]pyrrolidine-2- carboxamide |
| 112 | 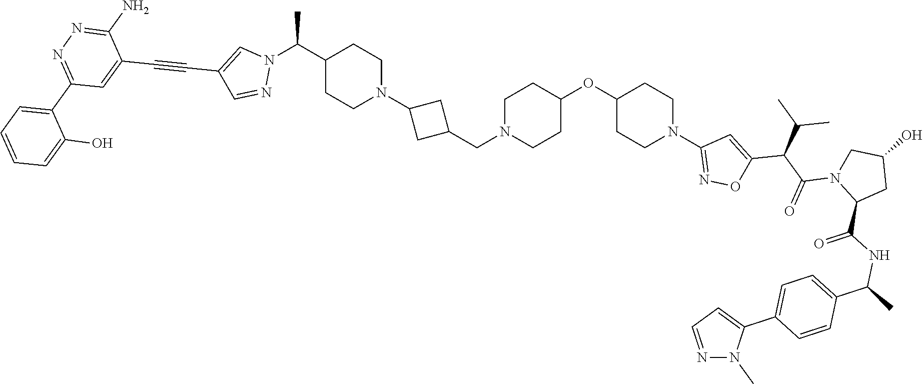 [SMILES: CC(C)[C@@H](C(=O)N1C[C@H](O)C[C@H]1C(=O)N[C@@H](C)c1ccc(-c2ccnn2C)cc1)c1cc(N2CCC(OC3CCN(CC4CC(N5CCC([C@H](C)n6cc(C#Cc7cc(-c8ccccc8O)nnc7N)cn6)CC5)C4)CC3)CC2)no1] | (2S,4R)-1-[(2R)-2-{3-[4-({1-[(3-{4-[(1S)- 1-(4-{2-[3-amino-6-(2- hydroxyphenyl)pyridazin-4-yl]ethynyl}- 1H-pyrazol-1-yl)ethyl]piperidin-1- yl}cyclobutyl)methyl]piperidin-4- yl}oxy)piperidin-1-yl]-1,2-oxazol-5-yl}-3- methylbutanoyl]-4-hydroxy-N-[(1S)-1-[4- (1-methyl-1H-pyrazol-5- yl)phenyl]ethyl]pyrrolidine-2- carboxamide |
| 113 | 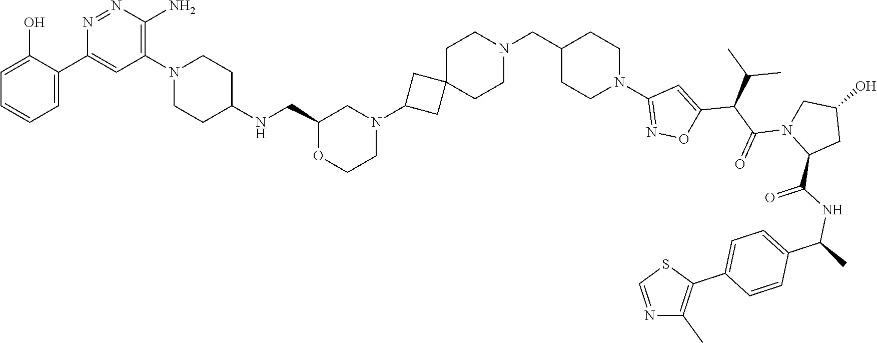 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(N4CCO[C@@H](CNC6CCN(c7cc(-c8ccccc8O)nnc7N)CC6)C4)C5)CC3)no2)C(C)C)cc1] | (2S,4R)-1-[(2R)-2-{3-[4-({2-[(2S)-2-[({1- [3-amino-6-(2-hydroxyphenyl)pyridazin- 4-yl]piperidin-4- yl}amino)methyl]morpholin-4-yl]-7- azaspiro[3.5]nonan-7-yl}methyl)piperidin- 1-yl]-1,2-oxazol-5-yl}-3-methylbutanoyl]- 4-hydroxy-N-[(1S)-1-[4-(4-methyl-1,3- thiazol-5-yl)phenyl]ethyl]pyrrolidine-2- carboxamide |
| 114 | 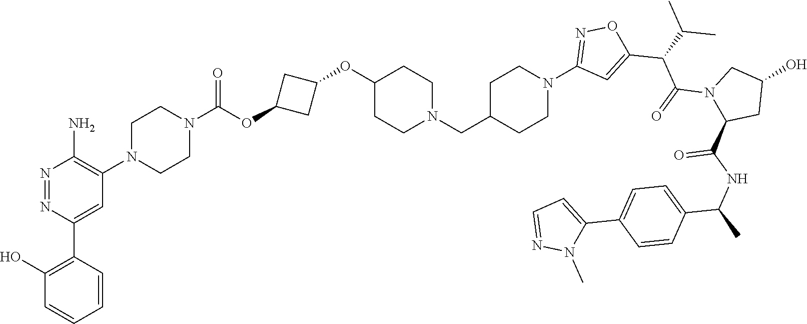 [SMILES: CC(C)[C@H](C(=O)N1C[C@H](O)C[C@H]1C(=O)N[C@@H](C)c1ccc(-c2ccnn2C)cc1)c1cc(N2CCC(CN3CCC(O[C@H]4C[C@H](OC(=O)N5CCN(c6cc(-c7ccccc7O)nnc6N)CC5)C4)CC3)CC2)no1] | (1r,3r)-3-({1-[(1-{5-[(2S)-1-[(2S,4R)-4- hydroxy-2-{[(1S)-1-[4-(1-methyl-1H- pyrazol-5- yl)phenyl]ethyl]carbamoyl}pyrrolidin-1- yl]-3-methyl-1-oxobutan-2-yl]-1,2-oxazol- 3-yl}piperidin-4-yl)methyl]piperidin-4- yl}oxy)cyclobutyl 4-[3-amino-6-(2- hydroxyphenyl)pyridazin-4-yl]piperazine- 1-carboxylate |
| 115 | 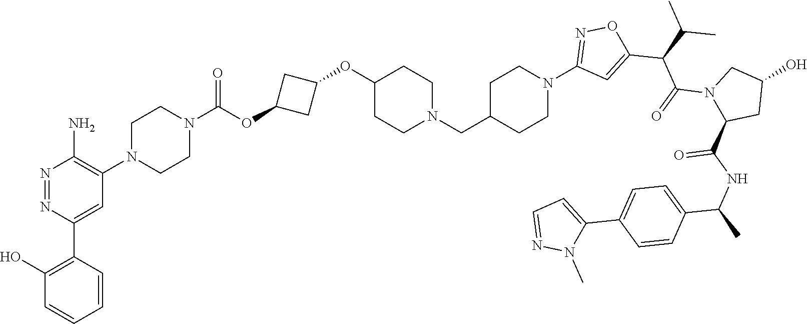 [SMILES: CC(C)[C@@H](C(=O)N1C[C@H](O)C[C@H]1C(=O)N[C@@H](C)c1ccc(-c2ccnn2C)cc1)c1cc(N2CCC(CN3CCC(O[C@H]4C[C@H](OC(=O)N5CCN(c6cc(-c7ccccc7O)nnc6N)CC5)C4)CC3)CC2)no1] | (1r,3r)-3-({1-[(1-{5-[(2R)-1-[(2S,4R)-4- hydroxy-2-{[(1S)-1-[4-(1-methyl-1H- pyrazol-5- yl)phenyl]ethyl]carbamoyl}pyrrolidin-1- yl]-3-methyl-1-oxobutan-2-yl]-1,2-oxazol- 3-yl}piperidin-4-yl)methyl]piperidin-4- yl}oxy)cyclobutyl 4-[3-amino-6-(2- hydroxyphenyl)pyridazin-4-yl]piperazine- 1-carboxylate |
| 116 | 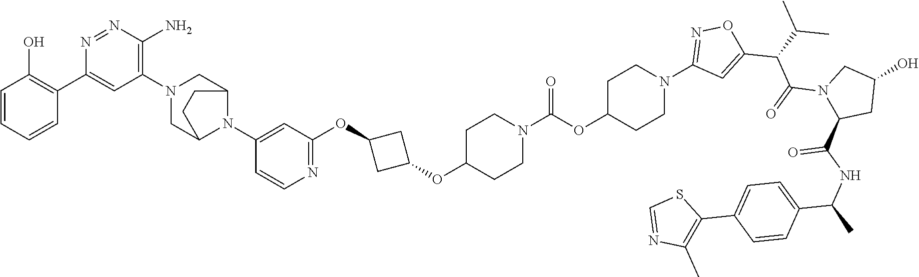 [SMILES: Cc1ncsc1-c1ccc([C@H](C)NC(=O)[C@@H]2C[C@@H](O)CN2C(=O)[C@H](c2cc(N3CCC(OC(=O)N4CCC(O[C@H]5C[C@H](Oc6cc(N7C8CCC7CN(c7cc(-c9ccccc9O)nnc7N)C8)ccn6)C5)CC4)CC3)no2)C(C)C)cc1] | 1-{5-[(2S)-1-[(2S,4R)-4-hydroxy-2- ([(1S)-1-[4-(4-methyl-1,3-thiazol-5- yl)phenyl]ethyl]carbamoyl}pyrrolidin-1- yl]-3-methyl-1-oxobutan-2-yl]-1,2-oxazol- 3-yl}piperidin-4-yl 4-[(1r,3r)-3-[(4-{3-[3- amino-6-(2-hydroxyphenyl)pyridazin-4- yl]-3,8-diazabicyclo[3.2.1]octan-8- yl}pyridin-2- yl)oxy]cyclobutoxy]piperidine-1- carboxylate |
| 117 | 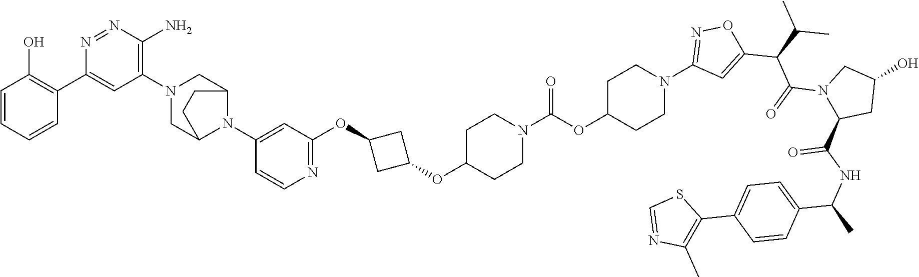 [SMILES: Cc1ncsc1-c1ccc([C@H](C)NC(=O)[C@@H]2C[C@@H](O)CN2C(=O)[C@@H](c2cc(N3CCC(OC(=O)N4CCC(O[C@H]5C[C@H](Oc6cc(N7C8CCC7CN(c7cc(-c9ccccc9O)nnc7N)C8)ccn6)C5)CC4)CC3)no2)C(C)C)cc1] | 1-{5-[(2R)-1-[(2S,4R)-4-hydroxy-2- {[(1S)-1-[4-(4-methyl-1,3-thiazol-5- yl)phenyl]ethyl]carbamoyl}pyrrolidin-1- yl]-3-methyl-1-oxobutan-2-yl]-1,2-oxazol- 3-yl}piperidin-4-yl 4-[(1r,3r)-3-[(4-{3-[3- amino-6-(2-hydroxyphenyl)pyridazin-4- yl]-3,8-diazabicyclo[3.2.1]octan-8- yl}pyridin-2- yl)oxy]cyclobutoxy]piperidine-1- carboxylate |
| 118 | 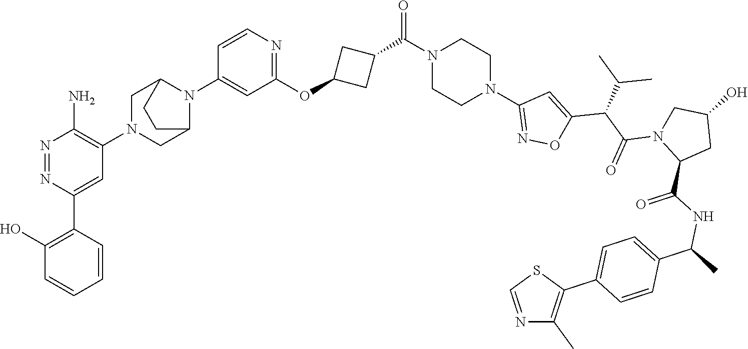 [SMILES: Cc1ncsc1-c1ccc([C@H](C)NC(=O)[C@@H]2C[C@@H](O)CN2C(=O)[C@H](c2cc(N3CCN(C(=O)[C@H]4C[C@H](Oc5cc(N6C7CCC6CN(c6cc(-c8ccccc8O)nnc6N)C7)ccn5)C4)CC3)no2)C(C)C)cc1] | (2S,4R)-4-hydroxy-N-[(1S)-1-[4-(4- methyl-1,3-thiazol-5-yl)phenyl]ethyl]-1- [(2S)-3-methyl-2-(3-{4-[(1r,3r)-3-[(4-{3- [3-amino-6-(2-hydroxyphenyl)pyridazin- 4-yl]-3,8-diazabicyclo[3.2.1]octan-8- yl}pyridin-2- yl)oxy]cyclobutanecarbonyl]piperazin-1- yl}-1,2-oxazol-5-yl)butanoyl]pyrrolidine- 2-carboxamide |
| 119 | 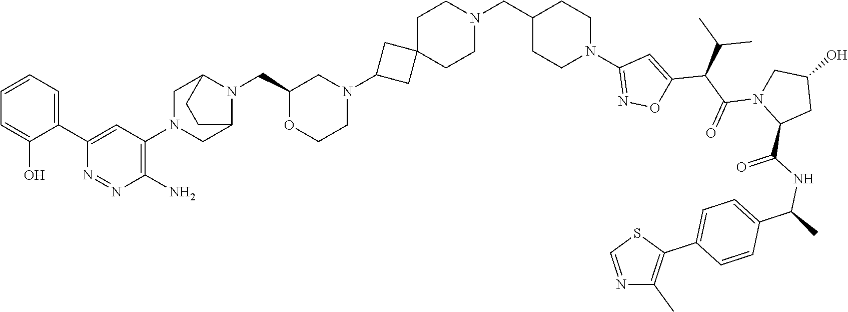 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(N4CCO[C@@H](CN6C7CCC6CN(c6cc(-c8ccccc8O)nnc6N)C7)C4)C5)CC3)no2)C(C)C)cc1] | (2S,4R)-1-[(2R)-2-{3-[4-({2-[(2R)-2-({3- [3-amino-6-(2-hydroxyphenyl)pyridazin- 4-yl]-3,8-diazabicyclo[3.2.1]octan-8- yl}methyl)morpholin-4-yl]-7- azaspiro[3.5]nonan-7-yl}methyl)piperidin- 1-yl]-1,2-oxazol-5-yl}-3-methylbutanoyl]- 4-hydroxy-N-[(1S)-1-[4-(4-methyl-1,3- thiazol-5-yl)phenyl]ethyl]pyrrolidine-2- carboxamide |
| 120 | 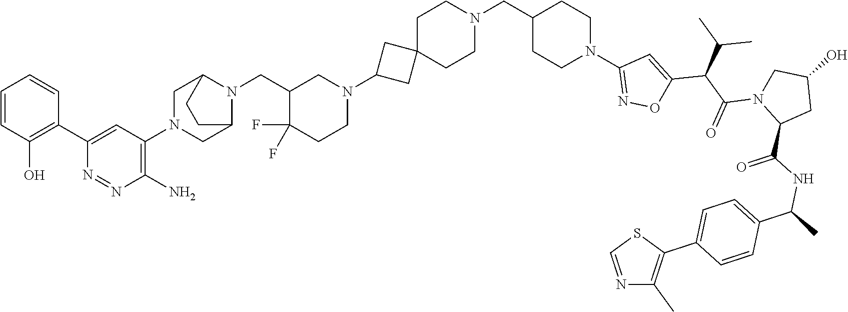 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(N4CCC(F)(F)C(CN6C7CCC6CN(c6cc(-c8ccccc8O)nnc6N)C7)C4)C5)CC3)no2)C(C)C)cc1] | (2S,4R)-1-[(2R)-2-{3-[4-({2-[3-({3-[3- amino-6-(2-hydroxyphenyl)pyridazin-4- yl]-3,8-diazabicyclo[3.2.1]octan-8- yl}methyl)-4,4-difluoropiperidin-1-yl]-7- azaspiro[3.5]nonan-7-yl}methyl)piperidin- 1-yl]-1,2-oxazol-5-yl}-3-methylbutanoyl]- 4-hydroxy-N-[(1S)-1-[4-(4-methyl-1,3- thiazol-5-yl)phenyl]ethyl]pyrrolidine-2- carboxamide |
| 121 | 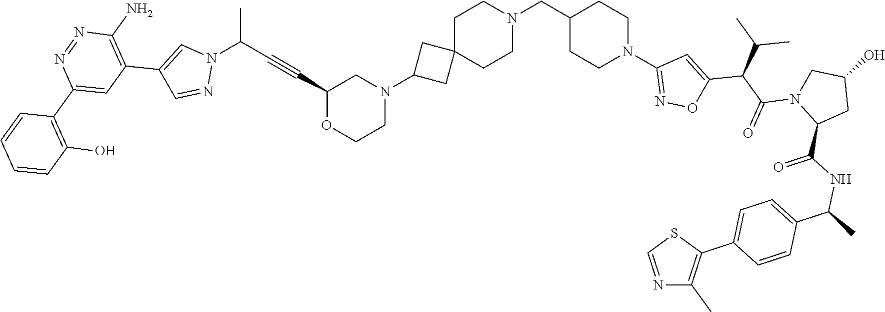 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(N4CCO[C@@H](C#CC(C)n6cc(-c7cc(-c8ccccc8O)nnc7N)cn6)C4)C5)CC3)no2)C(C)C)cc1] | (2S,4R)-1-[(2R)-2-{3-[4-({2-[(2S)-2-(3- {4-[3-amino-6-(2- hydroxyphenyl)pyridazin-4-yl]-1H- pyrazol-1-yl}but-1-yn-1-yl)morpholin-4- yl]-7-azaspiro[3.5]nonan-7- yl}methyl)piperidin-1-yl]-1,2-oxazol-5- yl}-3-methylbutanoyl]-4-hydroxy-N- [(1S)-1-[4-(4-methyl-1,3-thiazol-5- yl)phenyl]ethyl]pyrrolidine-2- carboxamide |
| 122 | 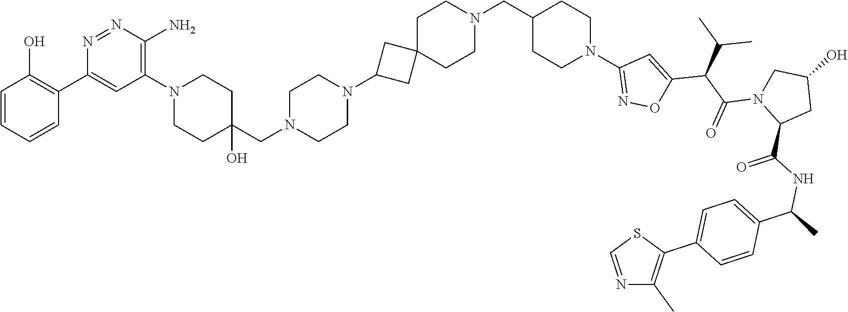 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(N4CCN(CC6(O)CCN(c7cc(-c8ccccc8O)nnc7N)CC6)CC4)C5)CC3)no2)C(C)C)cc1] | (2S,4R)-1-[(2R)-2-{3-[4-({2-[4-({1-[3- amino-6-(2-hydroxyphenyl)pyridazin-4- yl]-4-hydroxypiperidin-4- yl}methyl)piperazin-1-yl]-7- azaspiro[3.5]nonan-7-yl}methyl)piperidin- 1-yl]-1,2-oxazol-5-yl}-3-methylbutanoyl]- 4-hydroxy-N-[(1S)-1-[4-(4-methyl-1,3- thiazol-5-yl)phenyl]ethyl]pyrrolidine-2- carboxamide |
| 123 | 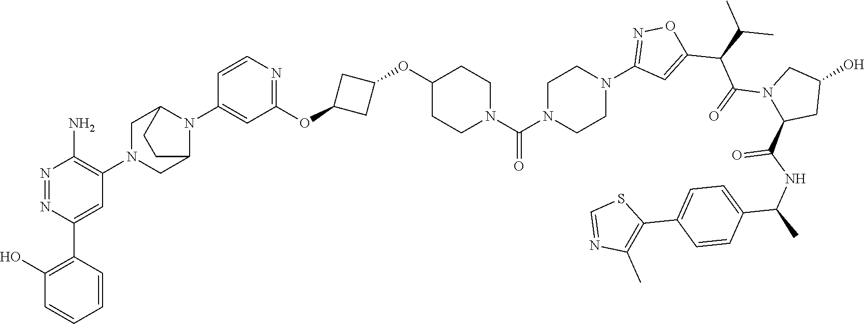 [SMILES: Cc1ncsc1-c1ccc([C@H](C)NC(=O)[C@@H]2C[C@@H](O)CN2C(=O)[C@@H](c2cc(N3CCN(C(=O)N4CCC(O[C@H]5C[C@H](Oc6cc(N7C8CCC7CN(c7cc(-c9ccccc9O)nnc7N)C8)ccn6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2S)-3-methyl-2-[3-(4-{4-[(1r,3r)-3-[(4- {3-[3-amino-6-(2- hydroxyphenyl)pyridazin-4-yl]-3,8- diazabicyclo[3.2.1]octan-8-yl}pyridin-2- yl)oxy]cyclobutoxy]piperidine-1- carbonyl}piperazin-1-yl)-1,2-oxazol-5- yl]butanoyl]pyrrolidine-2-carboxamide |
| 124 | 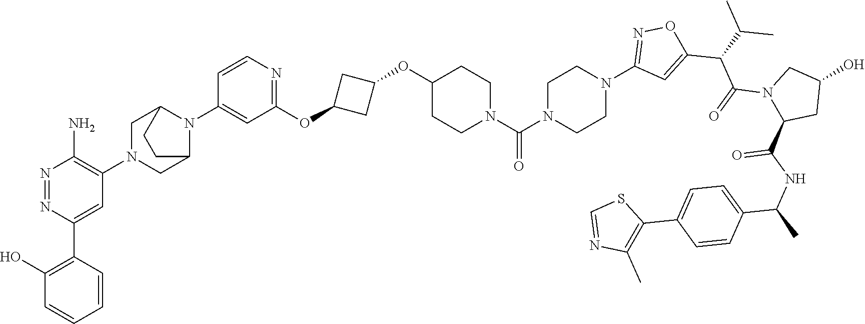 [SMILES: Cc1ncsc1-c1ccc([C@H](C)NC(=O)[C@@H]2C[C@@H](O)CN2C(=O)[C@H](c2cc(N3CCN(C(=O)N4CCC(O[C@H]5C[C@H](Oc6cc(N7C8CCC7CN(c7cc(-c9ccccc9O)nnc7N)C8)ccn6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-[3-(4-{4-[(1r,3r)-3-[(4- {3-[3-amino-6-(2- hydroxyphenyl)pyridazin-4-yl]-3,8- diazabicyclo[3.2.1]octan-8-yl}pyridin-2- yl)oxy]cyclobutoxy]piperidine-1- carbonyl}piperazin-1-yl)-1,2-oxazol-5- yl]butanoyl]pyrrolidine-2-carboxamide |
| 125 | 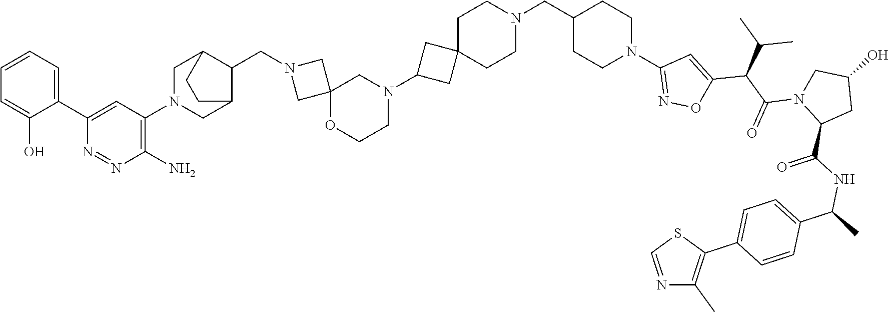 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(N4CCOC6(CN(CC7C8CCC7CN(c7cc(-c9ccccc9O)nnc7N)C8)C6)C4)C5)CC3)no2)C(C)C)cc1] | (2S,4R)-1-[(2R)-2-{3-[4-({2-[2-({3-[3- amino-6-(2-hydroxyphenyl)pyridazin-4- yl]-3-azabicyclo[3.2.1]octan-8- yl}methyl)-5-oxa-2,8- diazaspiro[3.5]nonan-8-yl]-7- azaspiro[3.5]nonan-7-yl}methyl)piperidin- 1-yl]-1,2-oxazol-5-yl}-3-methylbutanoyl]- 4-hydroxy-N-[(1S)-1-[4-(4-methyl-1,3- thiazol-5-yl)phenyl]ethyl]pyrrolidine-2- carboxamide |
| 126 | 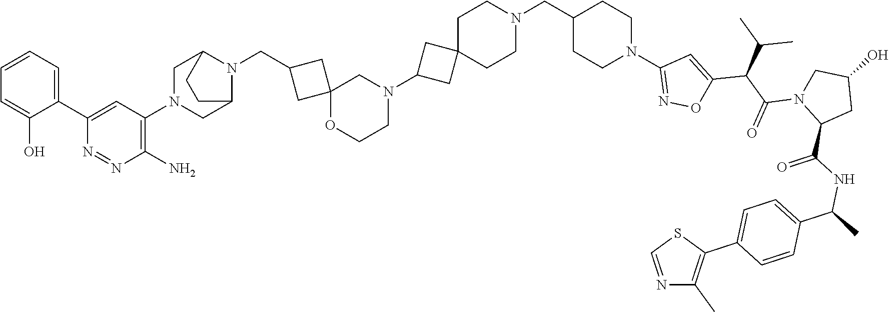 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(N4CCOC6(CC(CN7C8CCC7CN(c7cc(-c9ccccc9O)nnc7N)C8)C6)C4)C5)CC3)no2)C(C)C)cc1] | (2S,4R)-1-[(2R)-2-{3-[4-({2-[2-({3-[3- amino-6-(2-hydroxyphenyl)pyridazin-4- yl]-3,8-diazabicyclo[3.2.1]octan-8- yl}methyl)-5-oxa-8-azaspiro[3.5]nonan-8- yl]-7-azaspiro[3.5]nonan-7- yl}methyl)piperidin-1-yl]-1,2-oxazol-5- yl}-3-methylbutanoyl]-4-hydroxy-N- [(1S)-1-[4-(4-methyl-1,3-thiazol-5- yl)phenyl]ethyl]pyrrolidine-2- carboxamide |
| 127 | 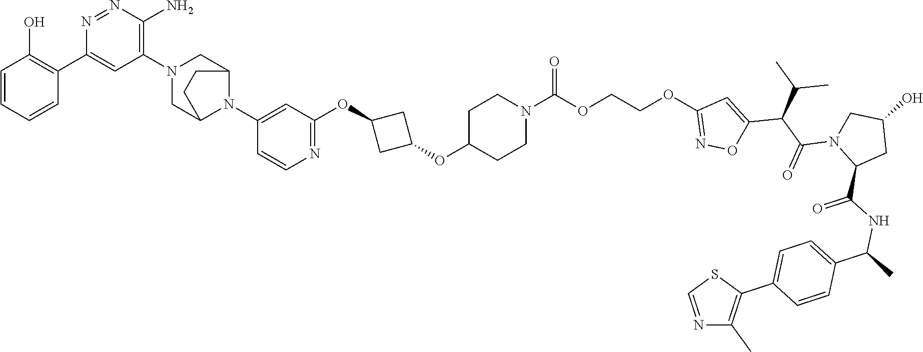 [SMILES: Cc1ncsc1-c1ccc([C@H](C)NC(=O)[C@@H]2C[C@@H](O)CN2C(=O)[C@@H](c2cc(OCCOC(=O)N3CCC(O[C@H]4C[C@H](Oc5cc(N6C7CCC6CN(c6cc(-c8ccccc8O)nnc6N)C7)ccn5)C4)CC3)no2)C(C)C)cc1] | 2-({5-[(2R)-1-[(2S,4R)-4-hydroxy-2- {[(1S)-1-[4-(4-methyl-1,3-thiazol-5- yl)phenyl]ethyl]carbamoyl}pyrrolidin-1- yl]-3-methyl-1-oxobutan-2-yl]-1,2-oxazol- 3-yl}oxy)ethyl 4-[(1r,3r)-3-[(4-{3-[3- amino-6-(2-hydroxyphenyl)pyridazin-4- yl]-3,8-diazabicyclo[3.2.1]octan-8- yl}pyridin-2- yl)oxy]cyclobutoxy]piperidine-1- carboxylate |
| 128 | 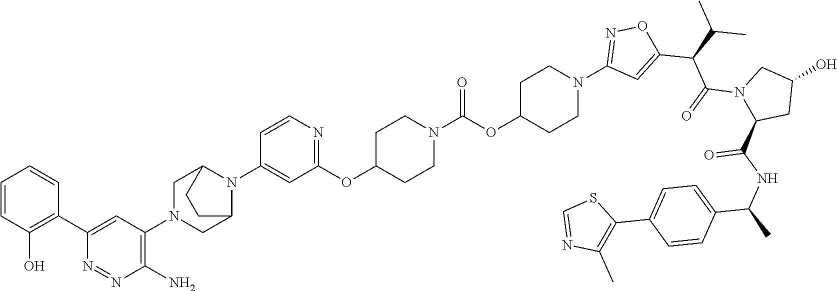 [SMILES: Cc1ncsc1-c1ccc([C@H](C)NC(=O)[C@@H]2C[C@@H](O)CN2C(=O)[C@@H](c2cc(N3CCC(OC(=O)N4CCC(Oc5cc(N6C7CCC6CN(c6cc(-c8ccccc8O)nnc6N)C7)ccn5)CC4)CC3)no2)C(C)C)cc1] | 1-(5-((R)-1-((2S,4R)-4-hydroxy-2-(((S)-1- (4-(4-methylthiazol-5- yl)phenyl)ethyl)carbamoyl)pyrrolidin-1- yl)-3-methyl-1-oxobutan-2-yl)isoxazol-3- yl)piperidin-4-yl 4-((4-(3-(3-amino-6-(2- hydroxyphenyl)pyridazin-4-yl)-3,8- diazabicyclo[3.2.1]octan-8-yl)pyridin-2- yl)oxy)piperidine-1-carboxylate |
| 129 | 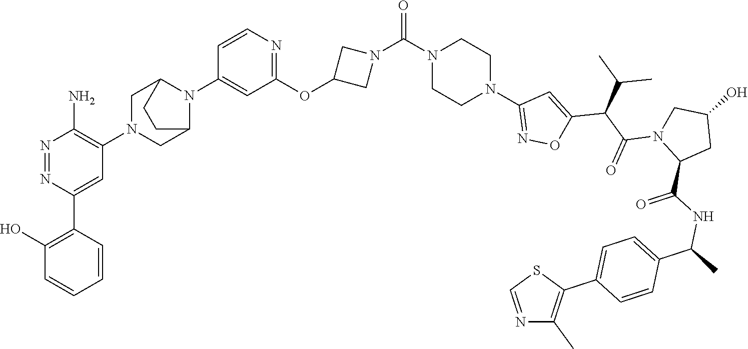 [SMILES: Cc1ncsc1-c1ccc([C@H](C)NC(=O)[C@@H]2C[C@@H](O)CN2C(=O)[C@@H](c2cc(N3CCN(C(=O)N4CC(Oc5cc(N6C7CCC6CN(c6cc(-c8ccccc8O)nnc6N)C7)ccn5)C4)CC3)no2)C(C)C)cc1] | (2S,4R)-1-[(2R)-2-[3-(4-{3-[(4-{3-[3- amino-6-(2-hydroxyphenyl)pyridazin-4- yl]-3,8-diazabicyclo[3.2.1]octan-8- yl}pyridin-2-yl)oxy]azetidine-1- carbonyl}piperazin-1-yl)-1,2-oxazol-5-yl]- 3-methylbutanoyl]-4-hydroxy-N-[(1S)-1- [4-(4-methyl-1,3-thiazol-5- yl)phenyl]ethyl]pyrrolidine-2- carboxamide |
| 130 | 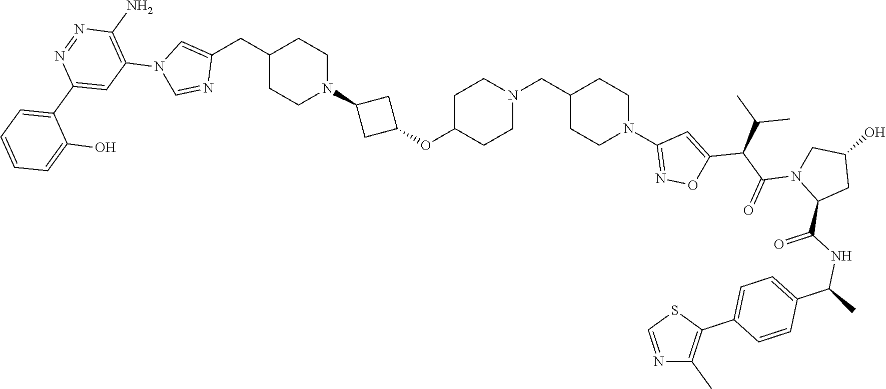 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCC(Cc7cn(-c8cc(-c9ccccc9O)nnc8N)cn7)CC6)C5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-{3-[4-({4-[(1r,3r)-3-[4- ({1-[3-amino-6-(2- hydroxyphenyl)pyridazin-4-yl]-1H- imidazol-4-yl}methyl)piperidin-1- yl]cyclobutoxy]piperidin-1- yl}methyl)piperidin-1-yl]-1,2-oxazol-5- yl}butanoyl]pyrrolidine-2-carboxamide |
| 131 | 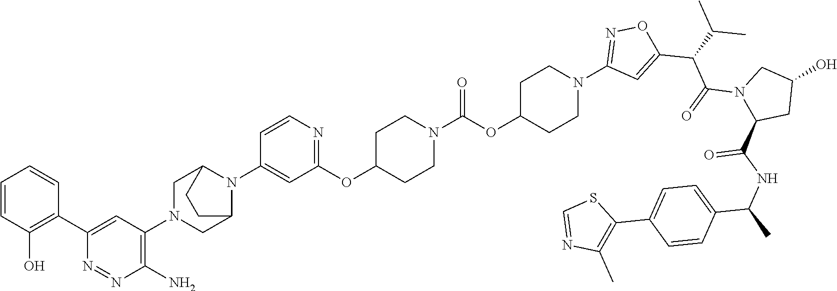 [SMILES: Cc1ncsc1-c1ccc([C@H](C)NC(=O)[C@@H]2C[C@@H](O)CN2C(=O)[C@H](c2cc(N3CCC(OC(=O)N4CCC(Oc5cc(N6C7CCC6CN(c6cc(-c8ccccc8O)nnc6N)C7)ccn5)CC4)CC3)no2)C(C)C)cc1] | 1-(5-((S)-1-((2S,4R)-4-hydroxy-2-(((S)-1- (4-(4-methylthiazol-5- yl)phenyl)ethyl)carbamoyl)pyrrolidin-1- yl)-3-methyl-1-oxobutan-2-yl)isoxazol-3- yl)piperidin-4-yl 4-((4-(3-(3-amino-6-(2- hydroxyphenyl)pyridazin-4-yl)-3,8- diazabicyclo[3.2.1]octan-8-yl)pyridin-2- yl)oxy)piperidine-1-carboxylate |
| 132 | 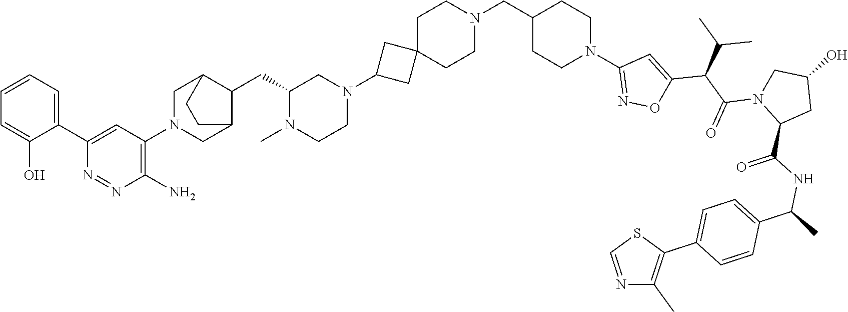 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(N4CCN(C)[C@H](CC6C7CCC6CN(c6cc(-c8ccccc8O)nnc6N)C7)C4)C5)CC3)no2)C(C)C)cc1] | (2S,4R)-1-[(2R)-2-{3-[4-({2-[(3S)-3-({3- [3-amino-6-(2-hydroxyphenyl)pyridazin- 4-yl]-3,8-diazabicyclo[3.2.1]octan-8- yl}methyl)-4-methylpiperazin-1-yl]-7- azaspiro[3.5]nonan-7-yl}methyl)piperidin- 1-yl]-1,2-oxazol-5-yl}-3-methylbutanoyl]- 4-hydroxy-N-[(1S)-1-[4-(4-methyl-1,3- thiazol-5-yl)phenyl]ethyl]pyrrolidine-2- carboxamide |
| 133 | 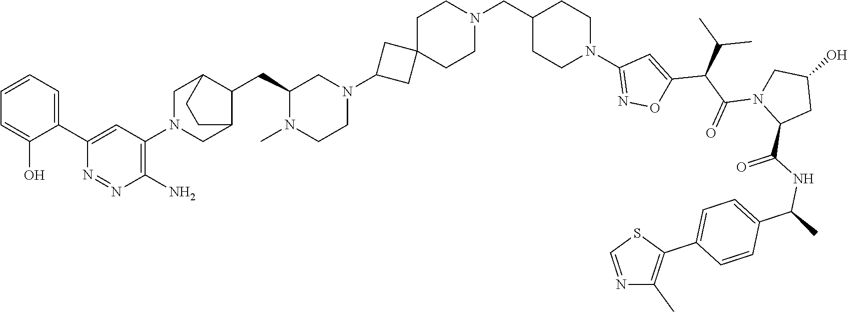 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(N4CCN(C)[C@@H](CC6C7CCC6CN(c6cc(-c8ccccc8O)nnc6N)C7)C4)C5)CC3)no2)C(C)C)cc1] | (2S,4R)-1-[(2R)-2-{3-[4-({2-[(3R)-3-({3- [3-amino-6-(2-hydroxyphenyl)pyridazin- 4-yl]-3,8-diazabicyclo[3.2.1]octan-8- yl}methyl)-4-methylpiperazin-1-yl]-7- azaspiro[3.5]nonan-7-yl}methyl)piperidin- 1-yl]-1,2-oxazol-5-yl}-3-methylbutanoyl]- 4-hydroxy-N-[(1S)-1-[4-(4-methyl-1,3- thiazol-5-yl)phenyl]ethyl]pyrrolidine-2- carboxamide |
| 134 | 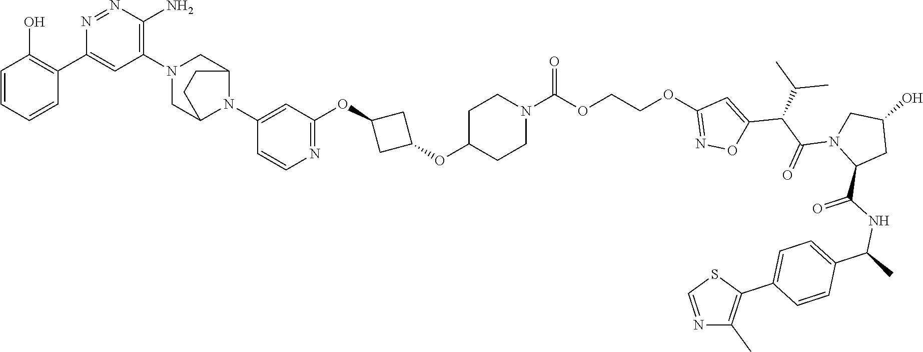 [SMILES: Cc1ncsc1-c1ccc([C@H](C)NC(=O)[C@@H]2C[C@@H](O)CN2C(=O)[C@H](c2cc(OCCOC(=O)N3CCC(O[C@H]4C[C@H](Oc5cc(N6C7CCC6CN(c6cc(-c8ccccc8O)nnc6N)C7)ccn5)C4)CC3)no2)C(C)C)cc1] | 2-({5-[(2S)-1-[(2S,4R)-4-hydroxy-2- {[(1S)-1-[4-(4-methyl-1,3-thiazol-5- yl)phenyl]ethyl]carbamoyl}pyrrolidin-1- yl]-3-methyl-1-oxobutan-2-yl]-1,2-oxazol- 3-yl}oxy)ethyl 4-[(1r,3r)-3-[(4-{3-[3- amino-6-(2-hydroxyphenyl)pyridazin-4- yl]-3,8-diazabicyclo[3.2.1]octan-8- yl}pyridin-2- yl)oxy]cyclobutoxy]piperidine-1- carboxylate |
| 135 | 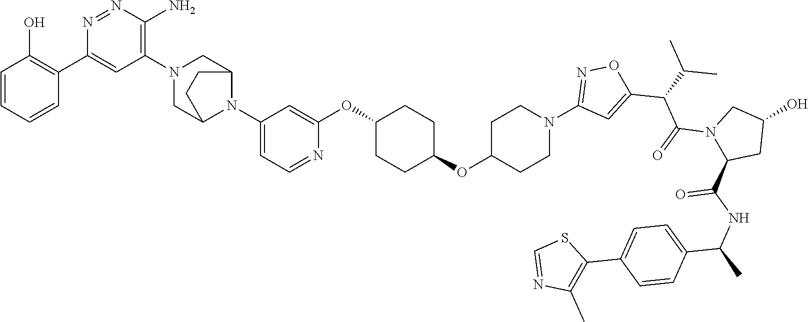 [SMILES: Cc1ncsc1-c1ccc([C@H](C)NC(=O)[C@@H]2C[C@@H](O)CN2C(=O)[C@H](c2cc(N3CCC(O[C@H]4CC[C@H](Oc5cc(N6C7CCC6CN(c6cc(-c8ccccc8O)nnc6N)C7)ccn5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2S)-3-methyl-2-[3-(4-{[(1r,4r)-4-[(4-{3- [3-amino-6-(2-hydroxyphenyl)pyridazin- 4-yl]-3,8-diazabicyclo[3.2.1]octan-8- yl}pyridin-2- yl)oxy]cyclohexyl]oxy}piperidin-1-yl)- 1,2-oxazol-5-yl]butanoyl]pyrrolidine-2- carboxamide |
| 136 | 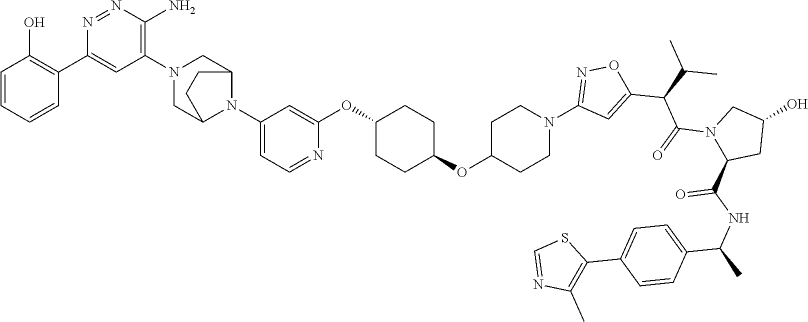 [SMILES: Cc1ncsc1-c1ccc([C@H](C)NC(=O)[C@@H]2C[C@@H](O)CN2C(=O)[C@@H](c2cc(N3CCC(O[C@H]4CC[C@H](Oc5cc(N6C7CCC6CN(c6cc(-c8ccccc8O)nnc6N)C7)ccn5)CC4)CC3)no2)C(C)C)cc1] | (2S,4R)-4-hydroxy-N-[(1S)-1-[4-(4- methyl-1,3-thiazol-5-yl)phenyl]ethyl]-1- [(2R)-3-methyl-2-[3-(4-{[(1r,4r)-4-[(4-{3- [3-amino-6-(2-hydroxyphenyl)pyridazin- 4-yl]-3,8-diazabicyclo[3.2.1]octan-8- yl}pyridin-2- yl)oxy]cyclohexyl]oxy}piperidin-1-yl)- 1,2-oxazol-5-yl]butanoyl]pyrrolidine-2- carboxamide |
| 137 | 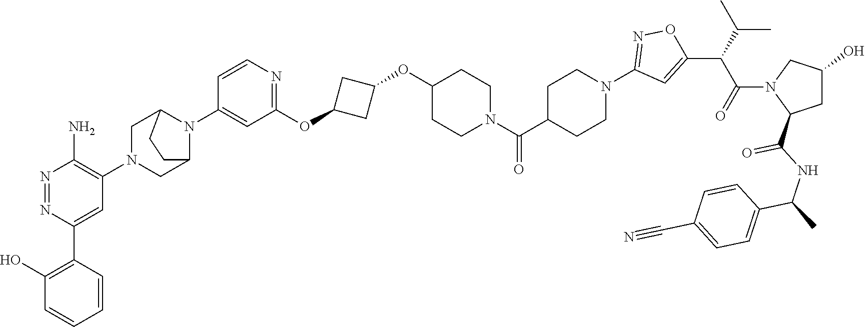 [SMILES: CC(C)[C@H](C(=O)N1C[C@H](O)C[C@H]1C(=O)N[C@@H](C)c1ccc(C#N)cc1)c1cc(N2CCC(C(=O)N3CCC(O[C@H]4C[C@H](Oc5cc(N6C7CCC6CN(c6cc(-c8ccccc8O)nnc6N)C7)ccn5)C4)CC3)CC2)no1] | (2S,4R)-N-[(1S)-1-(4-cyanophenyl)ethyl]- 4-hydroxy-1-[(2S)-3-methyl-2-[3-(4-{4- [(1r,3r)-3-[(4-{3-[3-amino-6-(2- hydroxyphenyl)pyridazin-4-yl]-3,8- diazabicyclo[3.2.1]octan-8-yl}pyridin-2- yl)oxy]cyclobutoxy]piperidine-1- carbonyl}piperidin-1-yl)-1,2-oxazol-5- yl]butanoyl]pyrrolidine-2-carboxamide |
| 138 | 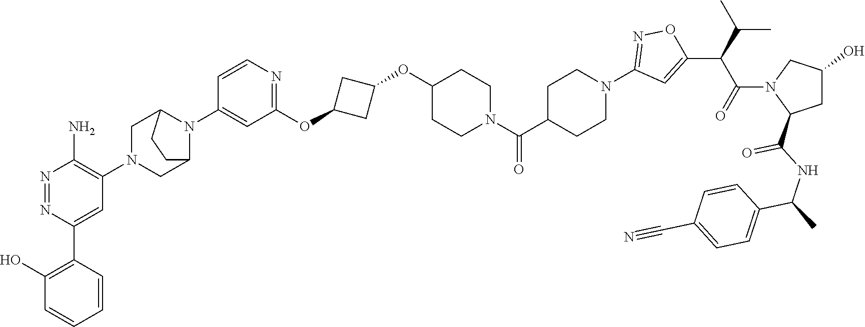 [SMILES: CC(C)[C@@H](C(=O)N1C[C@H](O)C[C@H]1C(=O)N[C@@H](C)c1ccc(C#N)cc1)c1cc(N2CCC(C(=O)N3CCC(O[C@H]4C[C@H](Oc5cc(N6C7CCC6CN(c6cc(-c8ccccc8O)nnc6N)C7)ccn5)C4)CC3)CC2)no1] | (2S,4R)-N-[(1S)-1-(4-cyanophenyl)ethyl]- 4-hydroxy-1-[(2R)-3-methyl-2-[3-(4-{4- [(1r,3r)-3-[(4-{3-[3-amino-6-(2- hydroxyphenyl)pyridazin-4-yl]-3,8- diazabicyclo[3.2.1]octan-8-yl}pyridin-2- yl)oxy]cyclobutoxy]piperidine-1- carbonyl}piperidin-1-yl)-1,2-oxazol-5- yl]butanoyl]pyrrolidine-2-carboxamide |
| 139 | 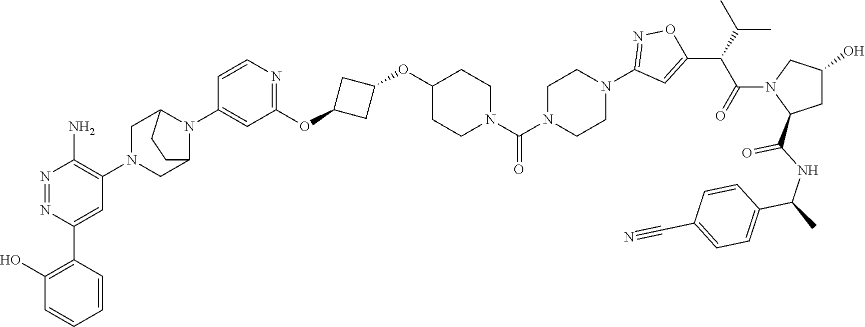 [SMILES: CC(C)[C@H](C(=O)N1C[C@H](O)C[C@H]1C(=O)N[C@@H](C)c1ccc(C#N)cc1)c1cc(N2CCN(C(=O)N3CCC(O[C@H]4C[C@H](Oc5cc(N6C7CCC6CN(c6cc(-c8ccccc8O)nnc6N)C7)ccn5)C4)CC3)CC2)no1] | (2S,4R)-N-[(1S)-1-(4-cyanophenyl)ethyl]- 4-hydroxy-1-[(2S)-3-methyl-2-[3-(4-{4- [(1r,3r)-3-[(4-{3-[3-amino-6-(2- hydroxyphenyl)pyridazin-4-yl]-3,8- diazabicyclo[3.2.1]octan-8-yl}pyridin-2- yl)oxy]cyclobutoxy]piperidine-1- carbonyl}piperazin-1-yl)-1,2-oxazol-5- yl]butanoyl]pyrrolidine-2-carboxamide |
| 140 | 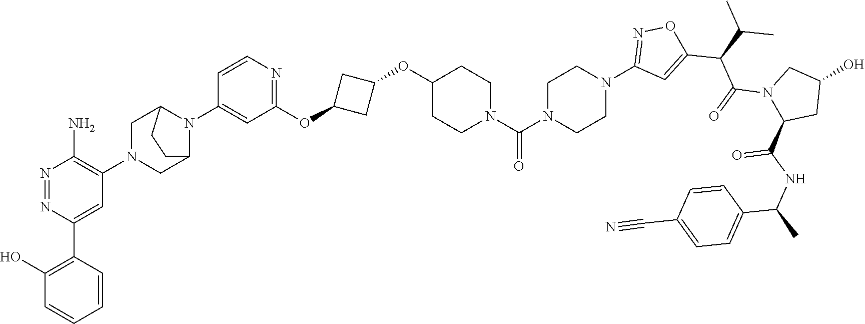 [SMILES: CC(C)[C@@H](C(=O)N1C[C@H](O)C[C@H]1C(=O)N[C@@H](C)c1ccc(C#N)cc1)c1cc(N2CCN(C(=O)N3CCC(O[C@H]4C[C@H](Oc5cc(N6C7CCC6CN(c6cc(-c8ccccc8O)nnc6N)C7)ccn5)C4)CC3)CC2)no1] | (2S,4R)-N-[(1S)-1-(4-cyanophenyl)ethyl]- 4-hydroxy-1-[(2R)-3-methyl-2-[3-(4-{4- [(1r,3r)-3-[(4-{3-[3-amino-6-(2- hydroxyphenyl)pyridazin-4-yl]-3,8- diazabicyclo[3.2.1]octan-8-yl}pyridin-2- yl)oxy]cyclobutoxy]piperidine-1- carbonyl}piperazin-1-yl)-1,2-oxazol-5- yl]butanoyl]pyrrolidine-2-carboxamide |
| 141 | 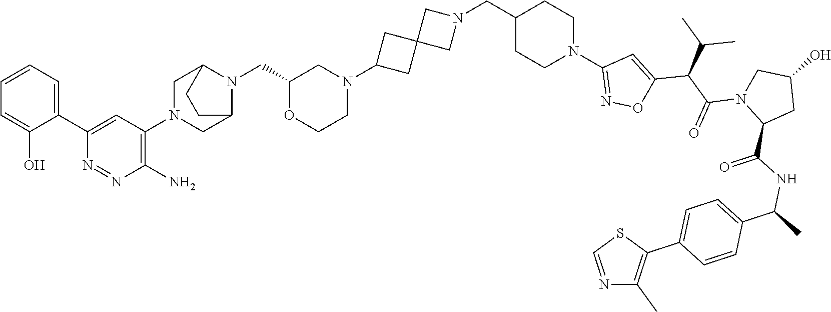 [SMILES: Cc1ncsc1-c1ccc([C@H](C)NC(=O)[C@@H]2C[C@@H](O)CN2C(=O)[C@@H](c2cc(N3CCC(CN4CC5(CC(N6CCO[C@H](CN7C8CCC7CN(c7cc(-c9ccccc9O)nnc7N)C8)C6)C5)C4)CC3)no2)C(C)C)cc1] | (2S,4R)-1-[(2R)-2-{3-[4-({6-[(2S)-2-({3- [3-amino-6-(2-hydroxyphenyl)pyridazin- 4-yl]-3,8-diazabicyclo[3.2.1]octan-8- yl}methyl)morpholin-4-yl]-2- azaspiro[3.3]heptan-2- yl}methyl)piperidin-1-yl]-1,2-oxazol-5- yl}-3-methylbutanoyl]-4-hydroxy-N- [(1S)-1-[4-(4-methyl-1,3-thiazol-5- yl)phenyl]ethyl]pyrrolidine-2- carboxamide |
| 142 | 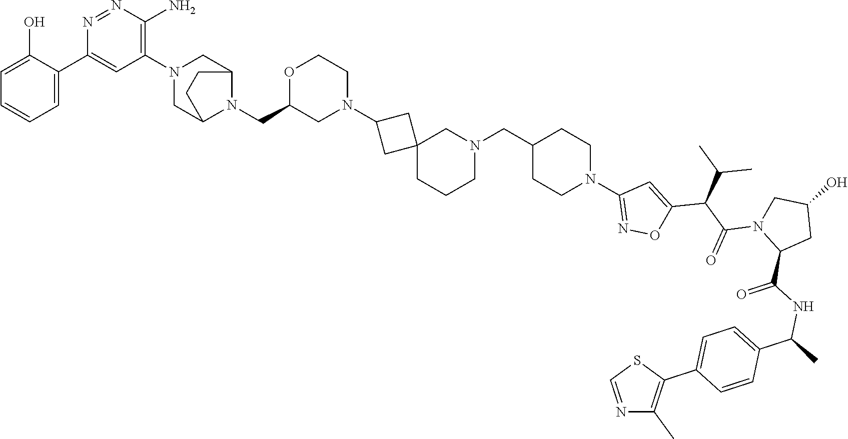 [SMILES: Cc1ncsc1-c1ccc([C@H](C)NC(=O)[C@@H]2C[C@@H](O)CN2C(=O)[C@@H](c2cc(N3CCC(CN4CCCC5(CC(N6CCO[C@H](CN7C8CCC7CN(c7cc(-c9ccccc9O)nnc7N)C8)C6)C5)C4)CC3)no2)C(C)C)cc1] | (2S,4R)-1-[(2R)-2-{3-[4-({2-[(2S)-2-({3- [3-amino-6-(2-hydroxyphenyl)pyridazin- 4-yl]-3,8-diazabicyclo[3.2.1]octan-8- yl}methyl)morpholin-4-yl]-6- azaspiro[3.5]nonan-6-yl}methyl)piperidin- 1-yl]-1,2-oxazol-5-yl}-3-methylbutanoyl]- 4-hydroxy-N-[(1S)-1-[4-(4-methyl-1,3- thiazol-5-yl)phenyl]ethyl]pyrrolidine-2- carboxamide |
| 143 | 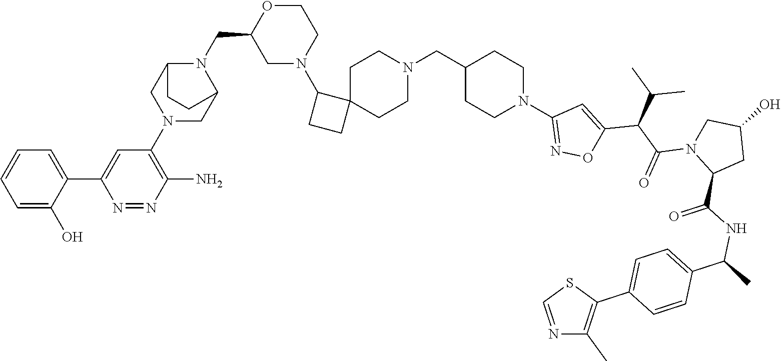 [SMILES: Cc1ncsc1-c1ccc([C@H](C)NC(=O)[C@@H]2C[C@@H](O)CN2C(=O)[C@@H](c2cc(N3CCC(CN4CCC5(CCC5N5CCO[C@H](CN6C7CCC6CN(c6cc(-c8ccccc8O)nnc6N)C7)C5)CC4)CC3)no2)C(C)C)cc1] | (2S,4R)-1-[(2R)-2-{3-[4-({1-[(2S)-2-({3- [3-amino-6-(2-hydroxyphenyl)pyridazin- 4-yl]-3,8-diazabicyclo[3.2.1]octan-8- yl}methyl)morpholin-4-yl]-7- azaspiro[3.5]nonan-7-yl}methyl)piperidin- 1-yl]-1,2-oxazol-5-yl}-3-methylbutanoyl]- 4-hydroxy-N-[(1S)-1-[4-(4-methyl-1,3- thiazol-5-yl)phenyl]ethyl]pyrrolidine-2- carboxamide |
| 144 | 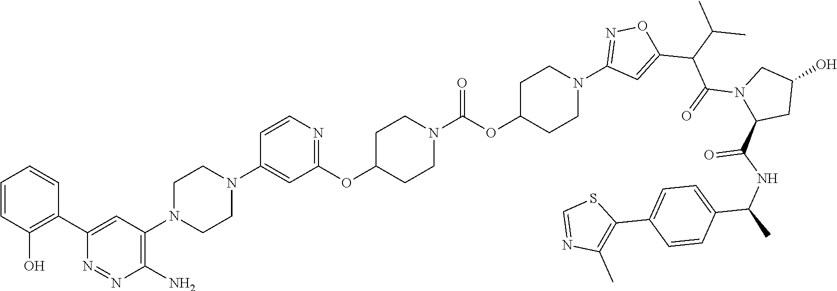 [SMILES: Cc1ncsc1-c1ccc([C@H](C)NC(=O)[C@@H]2C[C@@H](O)CN2C(=O)C(c2cc(N3CCC(OC(=O)N4CCC(Oc5cc(N6CCN(c7cc(-c8ccccc8O)nnc7N)CC6)ccn5)CC4)CC3)no2)C(C)C)cc1] | 1-(5-{1-[(2S,4R)-4-hydroxy-2-{[(1S)-1- [4-(4-methyl-1,3-thiazol-5- yl)phenyl]ethyl]carbamoyl}pyrrolidin-1- yl]-3-methyl-1-oxobutan-2-yl}-1,2- oxazol-3-yl)piperidin-4-yl 4-[(4-{4-[3- amino-6-(2-hydroxyphenyl)pyridazin-4- yl]piperazin-1-yl}pyridin-2- yl)oxy]piperidine-1-carboxylate |
| 145 | 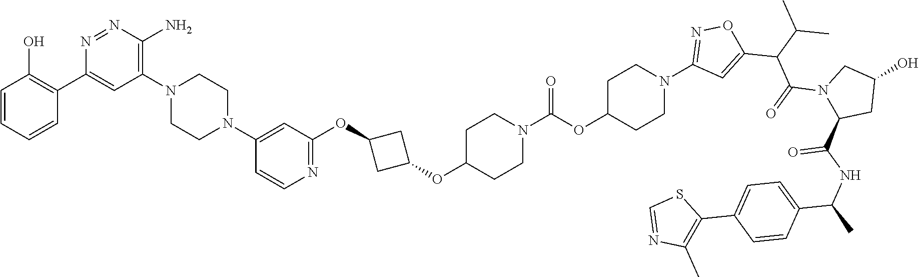 [SMILES: Cc1ncsc1-c1ccc([C@H](C)NC(=O)[C@@H]2C[C@@H](O)CN2C(=O)C(c2cc(N3CCC(OC(=O)N4CCC(O[C@H]5C[C@H](Oc6cc(N7CCN(c8cc(-c9ccccc9O)nnc8N)CC7)ccn6)C5)CC4)CC3)no2)C(C)C)cc1] | 1-(5-{1-[(2S,4R)-4-hydroxy-2-{[(1S)-1- [4-(4-methyl-1,3-thiazol-5- yl)phenyl]ethyl]carbamoyl}pyrrolidin-1- yl]-3-methyl-1-oxobutan-2-yl}-1,2- oxazol-3-yl)piperidin-4-yl 4-[(1r,3r)-3-[(4- {4-[3-amino-6-(2- hydroxyphenyl)pyridazin-4-yl]piperazin- 1-yl}pyridin-2- yl)oxy]cyclobutoxy]piperidine-1- carboxylate |
| 146 | 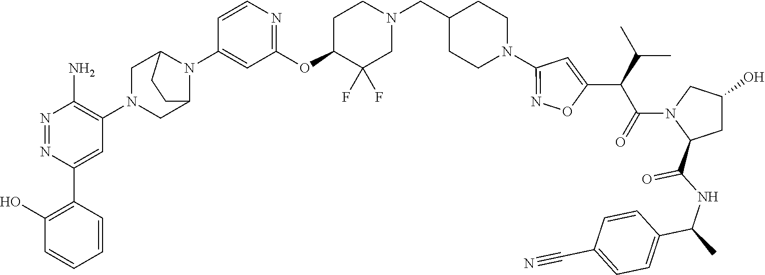 [SMILES: CC(C)[C@@H](C(=O)N1C[C@H](O)C[C@H]1C(=O)N[C@@H](C)c1ccc(C#N)cc1)c1cc(N2CCC(CN3CC[C@H](Oc4cc(N5C6CCC5CN(c5cc(-c7ccccc7O)nnc5N)C6)ccn4)C(F)(F)C3)CC2)no1] | (2S,4R)-1-[(2R)-2-[3-(4-{[(4S)-4-[(4-{3- [3-amino-6-(2-hydroxyphenyl)pyridazin- 4-yl]-3,8-diazabicyclo[3.2.1]octan-8- yl}pyridin-2-yl)oxy]-3,3- difluoropiperidin-1-yl]methyl}piperidin-1- yl)-1,2-oxazol-5-yl]-3-methylbutanoyl]-N- [(1S)-1-(4-cyanophenyl)ethyl]-4- hydroxypyrrolidine-2-carboxamide |
| 147 | 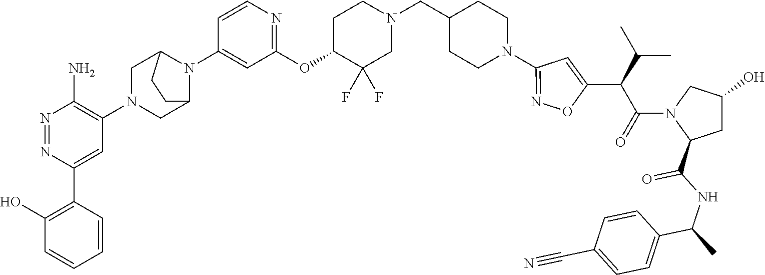 [SMILES: CC(C)[C@@H](C(=O)N1C[C@H](O)C[C@H]1C(=O)N[C@@H](C)c1ccc(C#N)cc1)c1cc(N2CCC(CN3CC[C@@H](Oc4cc(N5C6CCC5CN(c5cc(-c7ccccc7O)nnc5N)C6)ccn4)C(F)(F)C3)CC2)no1] | (2S,4R)-1-[(2R)-2-[3-(4-{[(4R)-4-[(4-{3- [3-amino-6-(2-hydroxyphenyl)pyridazin- 4-yl]-3,8-diazabicyclo[3.2.1]octan-8- yl}pyridin-2-yl)oxy]-3,3- difluoropiperidin-1-yl]methyl}piperidin-1- yl)-1,2-oxazol-5-yl]-3-methylbutanoyl]-N- [(1S)-1-(4-cyanophenyl)ethyl]-4- hydroxypyrrolidine-2-carboxamide |
| 148 | 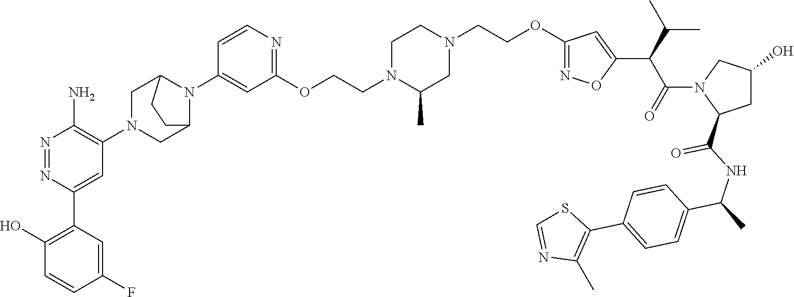 [SMILES: Cc1ncsc1-c1ccc([C@H](C)NC(=O)[C@@H]2C[C@@H](O)CN2C(=O)[C@@H](c2cc(OCCN3CCN(CCOc4cc(N5C6CCC5CN(c5cc(-c7cc(F)ccc7O)nnc5N)C6)ccn4)[C@H](C)C3)no2)C(C)C)cc1] | (2S,4R)-1-[(2R)-2-(3-{2-[(3R)-4-{2-[(4- {3-[3-amino-6-(5-fluoro-2- hydroxyphenyl)pyridazin-4-yl]-3,8- diazabicyclo[3.2.1]octan-8-yl}pyridin-2- yl)oxy]ethyl}-3-methylpiperazin-1- yl]ethoxy}-1,2-oxazol-5-yl)-3- methylbutanoyl]-4-hydroxy-N-[(1S)-1-[4- (4-methyl-1,3-thiazol-5- yl)phenyl]ethyl]pyrrolidine-2- carboxamide |
| 149 | 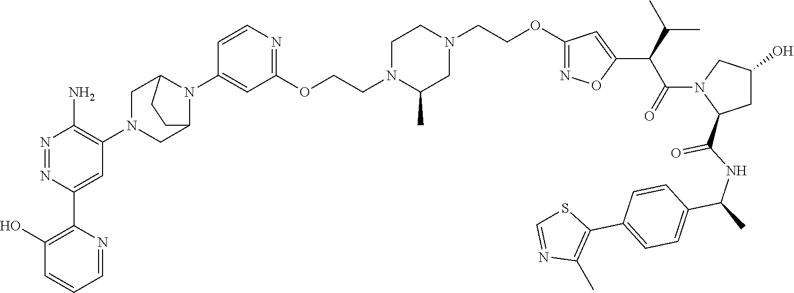 [SMILES: Cc1ncsc1-c1ccc([C@H](C)NC(=O)[C@@H]2C[C@@H](O)CN2C(=O)[C@@H](c2cc(OCCN3CCN(CCOc4cc(N5C6CCC5CN(c5cc(-c7ncccc7O)nnc5N)C6)ccn4)[C@H](C)C3)no2)C(C)C)cc1] | (2S,4R)-1-[(2R)-2-(3-{2-[(3R)-4-{2-[(4- {3-[3-amino-6-(3-hydroxypyridin-2- yl)pyridazin-4-yl]-3,8- diazabicyclo[3.2.1]octan-8-yl}pyridin-2- yl)oxy]ethyl}-3-methylpiperazin-1- yl]ethoxy}-1,2-oxazol-5-yl)-3- methylbutanoyl]-4-hydroxy-N-[(1S)-1-[4- (4-methyl-1,3-thiazol-5- yl)phenyl]ethyl]pyrrolidine-2- carboxamide |
| 150 | 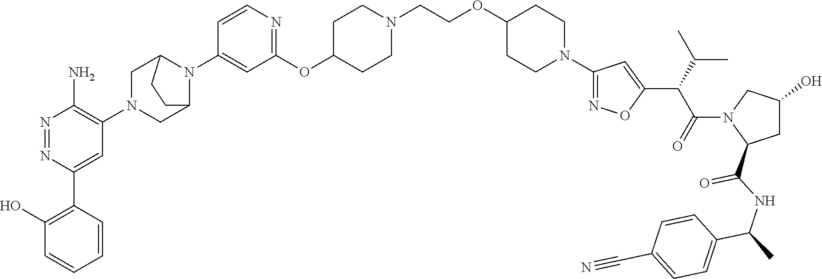 [SMILES: CC(C)[C@H](C(=O)N1C[C@H](O)C[C@H]1C(=O)N[C@@H](C)c1ccc(C#N)cc1)c1cc(N2CCC(OCCN3CCC(Oc4cc(N5C6CCC5CN(c5cc(-c7ccccc7O)nnc5N)C6)ccn4)CC3)CC2)no1] | (2S,4R)-1-[(2S)-2-{3-[4-(2-{4-[(4-{3-[3- amino-6-(2-hydroxyphenyl)pyridazin-4- yl]-3,8-diazabicyclo[3.2.1]octan-8- yl}pyridin-2-yl)oxy]piperidin-1- yl}ethoxy)piperidin-1-yl]-1,2-oxazol-5- yl}-3-methylbutanoyl]-N-[(1S)-1-(4- cyanophenyl)ethyl]-4-hydroxypyrrolidine- 2-carboxamide |
| 151 | 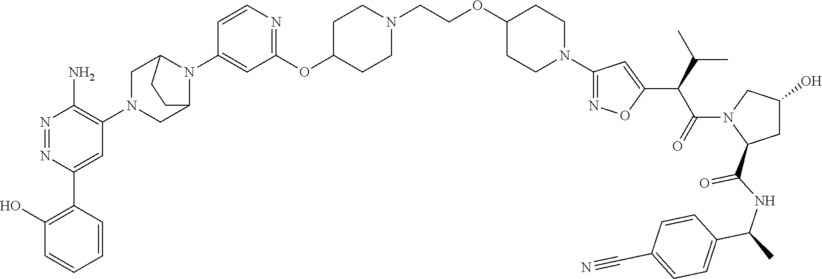 [SMILES: CC(C)[C@@H](C(=O)N1C[C@H](O)C[C@H]1C(=O)N[C@@H](C)c1ccc(C#N)cc1)c1cc(N2CCC(OCCN3CCC(Oc4cc(N5C6CCC5CN(c5cc(-c7ccccc7O)nnc5N)C6)ccn4)CC3)CC2)no1] | (2S,4R)-1-[(2R)-2-{3-[4-(2-{4-[(4-{3-[3- amino-6-(2-hydroxyphenyl)pyridazin-4- yl]-3,8-diazabicyclo[3.2.1]octan-8- yl}pyridin-2-yl)oxy]piperidin-1- yl}ethoxy)piperidin-1-yl]-1,2-oxazol-5- yl}-3-methylbutanoyl]-N-[(1S)-1-(4- cyanophenyl)ethyl]-4-hydroxypyrrolidine- 2-carboxamide |
| 152 | 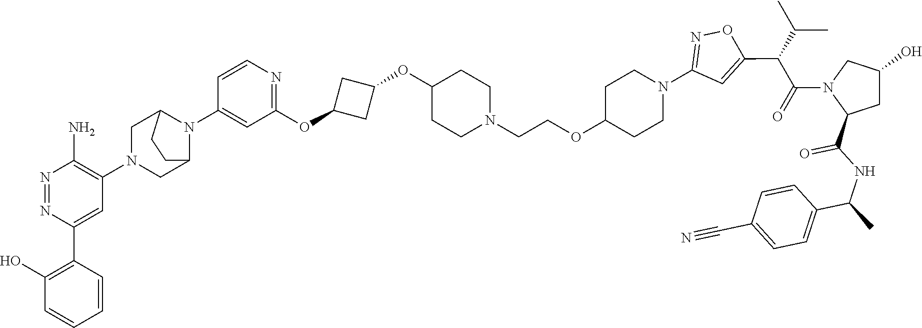 [SMILES: CC(C)[C@H](C(=O)N1C[C@H](O)C[C@H]1C(=O)N[C@@H](C)c1ccc(C#N)cc1)c1cc(N2CCC(OCCN3CCC(O[C@H]4C[C@H](Oc5cc(N6C7CCC6CN(c6cc(-c8ccccc8O)nnc6N)C7)ccn5)C4)CC3)CC2)no1] | (2S,4R)-N-[(1S)-1-(4-cyanophenyl)ethyl]- 4-hydroxy-1-[(2S)-3-methyl-2-{3-[4-(2- {4-[(1r,3r)-3-[(4-{3-[3-amino-6-(2- hydroxyphenyl)pyridazin-4-yl]-3,8- diazabicyclo[3.2.1]octan-8-yl}pyridin-2- yl)oxy]cyclobutoxy]piperidin-1- yl}ethoxy)piperidin-1-yl]-1,2-oxazol-5- yl}butanoyl]pyrrolidine-2-carboxamide |
| 153 | 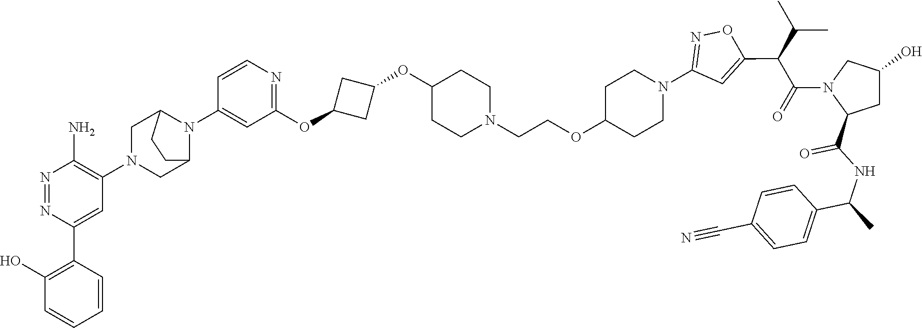 [SMILES: CC(C)[C@@H](C(=O)N1C[C@H](O)C[C@H]1C(=O)N[C@@H](C)c1ccc(C#N)cc1)c1cc(N2CCC(OCCN3CCC(O[C@H]4C[C@H](Oc5cc(N6C7CCC6CN(c6cc(-c8ccccc8O)nnc6N)C7)ccn5)C4)CC3)CC2)no1] | (2S,4R)-N-[(1S)-1-(4-cyanophenyl)ethyl]- 4-hydroxy-1-[(2R)-3-methyl-2-{3-[4-(2- {4-[(1r,3r)-3-[(4-{3-[3-amino-6-(2- hydroxyphenyl)pyridazin-4-yl]-3,8- diazabicyclo[3.2.1]octan-8-yl}pyridin-2- yl)oxy]cyclobutoxy]piperidin-1- yl}ethoxy)piperidin-1-yl]-1,2-oxazol-5- yl}butanoyl]pyrrolidine-2-carboxamide |
| 154 | 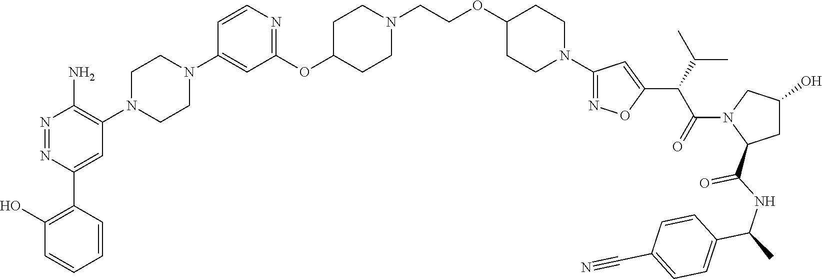 [SMILES: CC(C)[C@H](C(=O)N1C[C@H](O)C[C@H]1C(=O)N[C@@H](C)c1ccc(C#N)cc1)c1cc(N2CCC(OCCN3CCC(Oc4cc(N5CCN(c6cc(-c7ccccc7O)nnc6N)CC5)ccn4)CC3)CC2)no1] | (2S,4R)-1-[(2S)-2-{3-[4-(2-{4-[(4-{4-[3- amino-6-(2-hydroxyphenyl)pyridazin-4- yl]piperazin-1-yl}pyridin-2- yl)oxy]piperidin-1-yl}ethoxy)piperidin-1- yl]-1,2-oxazol-5-yl}-3-methylbutanoyl]- N-[(1S)-1-(4-cyanophenyl)ethyl]-4- hydroxypyrrolidine-2-carboxamide |
| 155 | 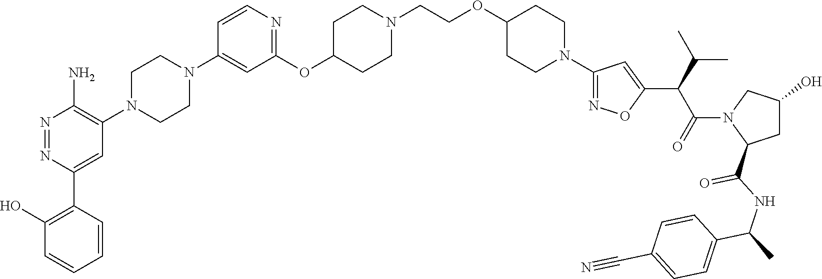 [SMILES: CC(C)[C@@H](C(=O)N1C[C@H](O)C[C@H]1C(=O)N[C@@H](C)c1ccc(C#N)cc1)c1cc(N2CCC(OCCN3CCC(Oc4cc(N5CCN(c6cc(-c7ccccc7O)nnc6N)CC5)ccn4)CC3)CC2)no1] | (2S,4R)-1-[(2R)-2-{3-[4-(2-{4-[(4-{4-[3- amino-6-(2-hydroxyphenyl)pyridazin-4- yl]piperazin-1-yl}pyridin-2- yl)oxy]piperidin-1-yl}ethoxy)piperidin-1- yl]-1,2-oxazol-5-yl}-3-methylbutanoyl]- N-[(1S)-1-(4-cyanophenyl)ethyl]-4- hydroxypyrrolidine-2-carboxamide |
| 156 | 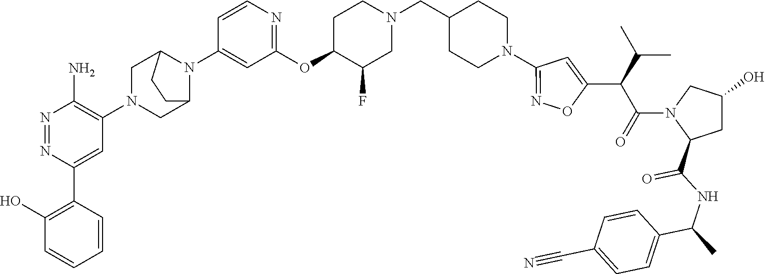 [SMILES: CC(C)[C@@H](C(=O)N1C[C@H](O)C[C@H]1C(=O)N[C@@H](C)c1ccc(C#N)cc1)c1cc(N2CCC(CN3CC[C@H](Oc4cc(N5C6CCC5CN(c5cc(-c7ccccc7O)nnc5N)C6)ccn4)[C@H](F)C3)CC2)no1] | (2S,4R)-1-[(2R)-2-[3-(4-{[(3R,4S)-4-[(4- {3-[3-amino-6-(2- hydroxyphenyl)pyridazin-4-yl]-3,8- diazabicyclo[3.2.1]octan-8-yl}pyridin-2- yl)oxy]-3-fluoropiperidin-1- yl]methyl}piperidin-1-yl)-1,2-oxazol-5- yl]-3-methylbutanoyl]-N-[(1S)-1-(4- cyanophenyl)ethyl]-4-hydroxypyrrolidine- 2-carboxamide |
| 157 | 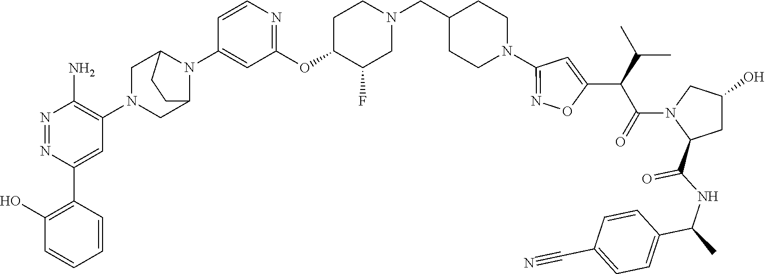 [SMILES: CC(C)[C@@H](C(=O)N1C[C@H](O)C[C@H]1C(=O)N[C@@H](C)c1ccc(C#N)cc1)c1cc(N2CCC(CN3CC[C@@H](Oc4cc(N5C6CCC5CN(c5cc(-c7ccccc7O)nnc5N)C6)ccn4)[C@@H](F)C3)CC2)no1] | (2S,4R)-1-[(2R)-2-[3-(4-{[(3S,4R)-4-[(4- {3-[3-amino-6-(2- hydroxyphenyl)pyridazin-4-yl]-3,8- diazabicyclo[3.2.1]octan-8-yl}pyridin-2- yl)oxy]-3-fluoropiperidin-1- yl]methyl}piperidin-1-yl)-1,2-oxazol-5- yl]-3-methylbutanoyl]-N-[(1S)-1-(4- cyanophenyl)ethyl]-4-hydroxypyrrolidine- 2-carboxamide |

TABLE 2
Target protein degradation via bifunctional degradation compounds of Table 1

| | DC₅₀ | Dₘₐₓ | | |
|---|---|---|---|---|
| Ex. | (nM)* | (%)** | MH+ | NMR transcript |
| 1 | D | NA | 1036.5 | |
| 2 | D | NA | 1078.6 | |
| 3 | D | NA | 1085.5 | |
| 4 | A | A | 1067.4 | |
| 5 | D | NA | 1136.4 | |
| 6 | A | A | 1095.7 | ¹H NMR: (400 MHz, DMSO-d₆) δ: 8.94-8.83 (m, 1H), 8.37 (s, 1H), |
| | | | | 8.33-8.19 (m, 1H), 8.11 (d, J = 2.8 Hz, 2H), 8.04 (s, 1H), 7.90 (dd, |
| | | | | J = 1.6, 8.4 Hz, 1H), 7.39-7.29 (m, 2H), 7.29-7.23 (m, 2H), 7.22- |
| | | | | 7.12 (m, 1H), 6.94-6.72 (m, 2H), 6.39 (s, 2H), 6.02 (s, 1H), 4.80 |
| | | | | (quin, J = 7.2 Hz, 1H), 4.27-4.23 (m, 1H), 4.21-4.12 (m, 2H), 3.75- |
| | | | | 3.66 (m, 1H), 3.62-3.58 (m, 1H), 3.57-3.44 (m, 3H), 3.38-3.17 (m, |
| | | | | 3H), 3.11-2.46 (m, 9H), 2.44-2.40 (m, 4H), 2.34 (s, 3H), 2.32-2.01 |
| | | | | (m, 4H), 1.97-1.51 (m, 11H), 1.50-1.11 (m, 11H), 1.04 (br d, J = 10.8 |
| | | | | Hz, 2H), 0.91-0.78 (m, 3H), 0.76-0.61 (m, 3H) |
| 7 | A | A | 1095.7 | ¹H NMR (400 MHz, DMSO-d₆) δ: 8.88 (s, 1H), 8.36 (s, 1H), 8.30 (d, |
| | | | | J = 7.6 Hz, 1H), 8.11 (s, 2H), 8.08 (s, 1H), 7.92 (dd, J = 1.6, 8.4 Hz, 1H), |
| | | | | 7.34 (d, J = 8.0 Hz, 2H), 7.30-7.24 (m, 2H), 7.17 (t, J = 7.2 Hz, 1H), |
| | | | | 6.91-6.75 (m, 2H), 6.39 (s, 2H), 6.09-5.95 (m, 1H), 4.81 (quin, |
| | | | | J = 7.2 Hz, 1H), 4.26 (br t, J = 7.6 Hz, 1H), 4.18 (br s, 2H), 4.12-4.07 |
| | | | | (m, 1H), 3.96 (br s, 1H), 3.61 (br dd, J = 4.4, 10.4 Hz, 1H), 3.55-3.41 |
| | | | | (m, 4H), 3.39-3.22 (m, 2H), 3.13 (br s, 1H), 2.93-2.84 (m, 1H), 2.77- |
| | | | | 2.68 (m, 2H), 2.63-2.53 (m, 3H), 2.35 (s, 3H), 2.12-1.78 (m, 10H), |
| | | | | 1.71-1.46 (m, 11H), 1.43-1.36 (m, 3H), 1.28 (br d, J = 6.8 Hz, 4H), |
| | | | | 1.15-0.93 (m, 5H), 0.89-0.81 (m, 3H), 0.74-0.64 (m, 3H) |
| 8 | A | A | 1095.7 | ¹H NMR: (400 MHz, DMSO-d₆) δ: 8.89 (s, 1H), 8.41-8.34 (m, 1H), |
| | | | | 8.34-8.19 (m, 1H), 8.11 (d, J = 7.2 Hz, 2H), 8.05 (s, 1H), 7.92 (dd, |
| | | | | J = 1.6, 8.4 Hz, 1H), 7.37-7.33 (m, 2H), 7.30-7.24 (m, 2H), 7.17 (t, |
| | | | | J = 7.2 Hz, 1H), 6.93-6.77 (m, 2H), 6.49-6.31 (m, 2H), 6.01 (s, 1H), |
| | | | | 4.81 (quin, J = 7.2 Hz, 1H), 4.26 (br t, J = 7.6 Hz, 1H), 4.21-4.16 (m, |
| | | | | 1H), 4.15-4.09 (m, 1H), 3.71-3.57 (m, 3H), 3.56-3.44 (m, 4H), |
| | | | | 3.37-3.27 (m, 2H), 3.22 (br d, J = 11.6 Hz, 2H), 2.87 (br d, J = 10.0 Hz, |
| | | | | 2H), 2.70-2.62 (m, 3H), 2.35 (s, 3H), 2.34-2.27 (m, 2H), 2.20-2.02 |
| | | | | (m, 5H), 1.97-1.85 (m, 2H), 1.78-1.54 (m, 11H), 1.42-1.37 (m, |
| | | | | 3H), 1.36-1.24 (m, 5H), 1.21-0.95 (m, 5H), 0.90-0.81 (m, 3H), |
| | | | | 0.74-0.64 (m, 3H) |
| 9 | A | A | 1095.7 | ¹H NMR: (400 MHz, DMSO-d₆) δ: 8.99 (s, 1H), 8.46 (s, 1H), 8.40 (d, |
| | | | | J = 7.6 Hz, 1H), 8.22 (s, 2H), 8.18 (s, 1H), 8.02 (dd, J = 1.6, 8.4 Hz, 1H), |
| | | | | 7.48-7.41 (m, 2H), 7.41-7.34 (m, 2H), 7.27 (t, J = 7.6 Hz, 1H), 6.97- |
| | | | | 6.90 (m, 2H), 6.52-6.44 (m, 2H), 6.13-6.09 (m, 1H), 4.92 (br t, |
| | | | | J = 7.2 Hz, 1H), 4.37 (t, J = 7.6 Hz, 1H), 4.29 (br s, 1H), 4.25-4.13 (m, |
| | | | | 1H), 4.06 (br s, 1H), 3.77-3.52 (m, 4H), 3.46-3.36(m, 7H), 2.91 (br |
| | | | | d, J = 10.0 Hz, 2H), 2.76-2.72 (m, 2H), 2.46 (s, 3H), 2.26-2.18 (m, |
| | | | | 2H), 2.07-1.86(m, 8H), 1.82-1.60 (m, 10H), 1.53-1.47 (m, 3H), |
| | | | | 1.38 (br d, J = 7.2 Hz, 4H), 1.27-1.04 (m, 6H), 1.00-0.91 (m, 3H), |
| | | | | 0.86-0.74 (m, 3H) |
| 10 | A | A | 1120.7 | |
| 11 | A | A | 1141.4 | ¹H NMR: (400 MHz, DMSO-d₆) δ: 14.23 (br s, 1H), 8.98 (s, 1H), |
| | | | | 8.39 (br d, J = 7.6 Hz, 1H), 7.95 (br d, J = 8.0 Hz, 1H), 7.51 (s, 1H), |
| | | | | 7.44 (br d, J = 7.6 Hz, 2H), 7.40-7.33 (m, 2H), 7.27-7.20 (m, 1H), |
| | | | | 6.88 (br d, J = 8.0 Hz, 2H), 6.10 (s, 1H), 5.88 (br s, 2H), 5.10 (br s, |
| | | | | 1H), 4.91 (s, 1H), 4.36 (br t, J = 7.6 Hz, 1H), 4.28 (br s, 1H), 4.13- |
| | | | | 4.01 (m, 1H), 3.84 (s, 1H), 3.71 (br dd, J = 4.0, 9.6 Hz, 1H), 3.65- |
| | | | | 3.49 (m, 3H), 3.41 (br d, J = 9.6 Hz, 1H), 3.27-3.18 (m, 5H), 2.99 (br |
| | | | | s, 2H), 2.79-2.61 (m, 5H), 2.45 (s, 3H), 2.22 (br s, 4H), 2.14-2.00 |
| | | | | (m, 7H), 1.99-1.80 (m, 9H), 1.79-1.54 (m, 8H), 1.48-1.29 (m, 7H), |
| | | | | 1.08 (br d, J = 10.8 Hz, 2H), 0.94 (br d, J = 6.8 Hz, 3H), 0.78 (br d, |
| | | | | J = 6.8 Hz, 3H) |
| 12 | D | NA | 1187.4 | ¹H NMR: (400 MHz, DMSO-d₆) δ: 9.02-8.89 (m, 1H), 8.28-8.16 |
| | | | | (m, 2H), 7.91 (d, J = 6.8 Hz, 1H), 7.76 (d, J = 6.0 Hz, 1H), 7.67-7.58 |
| | | | | (m, 1H), 7.52-7.37 (m, 4H), 7.40-7.29 (m, 1H), 7.26-7.18 (m, 1H), |
| | | | | 7.11-7.01 (m, 1H), 6.91-6.79 (m, 2H), 6.52 (dd, J = 2.0 Hz, J = 6.0 |
| | | | | Hz, 1H), 6.20 (s, 1H), 6.13 (d, J = 1.6 Hz, 1H), 6.02-5.91 (m, 2H), |
| | | | | 5.23-5.15 (m, 1H), 4.98-4.80 (m, 1H), 4.56-4.42 (m, 3H), 4.31- |
| | | | | 4.24 (m, 2H), 4.15 (s, 1H), 3.75-3.68 (m, 2H), 3.53 (d, J = 4.4 Hz, |
| | | | | 1H), 3.31 (s, 3H), 3.26 (s, 2H), 3.27-3.24 (m, 1H), 3.01 (d, J = 11.6 |
| | | | | Hz, 2H), 2.96-2.79 (m, 3H), 2.73 (s, 2H), 2.47-2.42 (m, 4H), 2.35- |
| | | | | 2.22 (m, 6H), 2.17 (d, J = 7.2 Hz, 2H), 2.10-2.04 (m, 3H), 2.01-1.93 |
| | | | | (m, 4H), 1.87-1.71 (m, 3H), 1.48-1.21 (m, 6H), 0.96 (d, J = 6.4 Hz, |
| | | | | 2H), 0.88-0.73 (m, 4H) |
| 13 | D | NA | 1147.5 | ¹H NMR: (400 MHz, MeOD-d₄) δ: 8.87 (s, 1H), 7.81-7.71 (m, 2H), |
| | | | | 7.49-7.36 (m,5H), 7.25 (t, J = 7.6 Hz, 1H), 6.95-6.87 (m, 2H), 6.60 |
| | | | | (d, J = 6.4 Hz, 1H), 6.18-6.09 (m, 2H), 5.14 (br s, 1H), 5.07-4.93 |
| | | | | (m, 1H), 4.83-4.55 (m, 3H), 4.48 (br s, 1H), 4.39-4.37 (m, 1H), 3.75- |
| | | | | 3.64 (m, 5H), 3.44-3.35 (m, 2H), 3.29-3.04 (m, 2H), 2.95 (br s, |
| | | | | 2H), 2.82-2.76 (m, 2H), 2.64 (br s, 3H), 2.55-2.36 (m, 5H), 2.34- |
| | | | | 2.12 (m, 6H), 2.05-1.94 (m, 1H), 1.85 (br s, 2H), 1.75-1.55 (m, 5H), |
| | | | | 1.39 (br d, J = 11.6 Hz, 3H), 1.40-1.31 (s, 4H), 1.07 (d, J = 6.8 Hz, |
| | | | | 3H), 1.01-0.83 (m, 3H), 0.74 (br s, 2H), 0.59 (br s, 2H) |
| 14 | A | A | 1063.5 | |
| 15 | B | A | 1049.5 | |
| 16 | A | A | 1063.7 | |
| 17 | A | A | 1035.7 | |
| 18 | D | NA | 1147.7 | |
| 19 | A | A | 1145.7 | ¹H NMR: (400 MHz, DMSO-d₆) δ: 8.98 (s, 1H), 8.39 (d, J = 7.6 Hz, |
| | | | | 1H), 8.14 (s, 1H), 7.92-7.90 (m, 1H), 7.77-7.75 (m, 1H), 7.49-7.47 |
| | | | | (m, 1H), 7.45-7.43 (m, 2H), 7.38-7.36 (m, 2H), 7.24-7.20 (m, 1H), |
| | | | | 6.88-6.82 (m, 2H), 6.60-6.46 (m, 2H), 6.14-6.11 (m, 2H), 5.99- |
| | | | | 5.96 (m, 2H), 5.36-5.33 (m, 1H), 5.10-4.89 (m, 2H), 4.49 (s, 2H), |
| | | | | 4.36 (t, J = 8.0 Hz, 1H), 4.28 (s, 1H), 3.73-3.69 (m, 1H), 3.62-3.54 |
| | | | | (m, 4H), 3.42-3.40 (m, 3H), 3.26-3.23 (m, 3H), 3.01 (d, J = 7.2 Hz, |
| | | | | 2H), 2.75-2.69 (m, 5H), 2.45 (s, 3H), 2.25-2.13 (m, 3H), 2.04-1.93 |
| | | | | (m, 5H), 1.82-1.69 (m, 8H), 1.46-1.37 (m, 3H), 1.12-1.07 (m, 2H), |
| | | | | 0.97-0.94 (m, 3H), 0.83-0.77 (m, 3H) |
| 20 | A | A | 1150.7 | ¹H NMR: (400 MHz, DMSO-d₆) δ: 9.02-8.80 (m, 1H), 8.39 (d, J = |
| | | | | 8.0 Hz, 1H), 7.96 (d, J = 7.2 Hz, 1H), 7.56-7.34 (m, 5H), 7.29-7.20 |
| | | | | (m, 1H), 6.93-6.81 (m, 2H), 6.15 (s, 1H), 6.06-5.84 (m, 2H), 5.15- |
| | | | | 4.99 (m, 1H), 4.95-4.87 (m, 1H), 4.39-4.24 (m, 2H), 4.12 (s, 1H), |
| | | | | 3.75-3.52 (m, 4H), 3.46-3.39 (m, 2H), 3.38-3.34 (m, 3H), 3.26 (s, |
| | | | | 6H), 2.97 (d, J = 10.4 Hz, 6H), 2.83-2.73 (m, 3H), 2.52-2.52 (m, |
| | | | | 1H), 2.58-2.51 (m, 5H), 2.45 (s, 3H), 2.26-2.11 (m, 3H), 2.10-1.73 |
| | | | | (m, 13H), 1.58 (s, 3H), 1.48-1.34 (m, 3H), 1.21 (d, J = 7.6 Hz, 2H), |
| | | | | 1.01-0.91 (m, 3H), 0.86-0.75 (m, 3H) |
| 21 | A | A | 1147.7 | |
| 22 | A | A | 1153.8 | ¹H NMR: (400 MHz, DMSO-d₆) δ: 8.89 (s, 1H), 8.30 (d, J = 7.6 Hz, |
| | | | | 1H), 8.06 (s, 2H), 7.84 (d, J = 8.4 Hz, 1H), 7.39-7.31 (m, 3H), 7.31- |
| | | | | 7.24 (m, 2H), 7.15 (t, J = 7.6 Hz, 1H), 6.84-6.75 (m, 2H), 6.44 (br s, |
| | | | | 1H), 6.01 (s, 1H), 5.81-5.71 (m, 2H), 5.01 (br s, 1H), 4.82 (quin, |
| | | | | J = 7.2 Hz, 1H), 4.26 (br t, J = 7.6 Hz, 1H), 4.19 (br s, 1H), 4.02 (br s, |
| | | | | 2H), 3.62 (br dd, J = 4.4, 10.8 Hz, 1H), 3.56-3.49 (m, 5H), 2.97 (br s, |
| | | | | 4H), 2.73 (br d, J = 10.0 Hz, 3H), 2.68-2.57 (m, 5H), 2.36 (s, 3H), |
| | | | | 2.17-1.98 (m, 5H), 1.96-1.77 (m, 10H), 1.73-1.52 (m, 8H), 1.39- |
| | | | | 1.24 (m, 6H), 1.23-0.95 (m, 5H), 0.89-0.77 (m, 9H), 0.79-0.67 (m, |
| | | | | 3H) |
| 23 | A | A | 1127.7 | ¹H NMR: (400 MHz, DMSO-d₆) δ: 8.98 (s, 1H), 8.39 (d, J = 7.6 Hz, |
| | | | | 1H), 8.24 (s, 2H), 7.98-7.91 (m, 1H), 7.50 (s, 1H), 7.44 (d, J = 8.0 Hz, |
| | | | | 2H), 7.39-7.34 (m, 2H), 7.27-7.21 (m, 1H), 6.90-6.85 (m, 2H), |
| | | | | 6.10 (s, 1H), 5.87 (s, 2H), 4.97-4.85 (m, 1H), 4.36 (m, 1H), 4.28 (s, |
| | | | | 1H), 4.09 (d, J = 5.6 Hz, 1H), 3.78 (d, J = 11.2 Hz, 2H), 3.73-3.66 (m, |
| | | | | 2H), 3.63-3.56 (m, 5H), 3.54 (s, 2H), 3.28-3.20 (m, 2H), 2.96-2.88 |
| | | | | (m, 4H), 2.76-2.69 (m, 3H), 2.66-2.60 (m, 1H), 2.45 (s, 4H), 2.29 |
| | | | | (d, J = 6.0 Hz, 1H), 2.25-2.20 (m, 1H), 2.18-2.06 (m, 5H), 2.00- |
| | | | | 1.85 (m, 9H), 1.81-1.66 (m, 7H), 1.62-1.53 (m, 2H), 1.51-1.30 (m, |
| | | | | 6H), 1.11-1.02 (m, 2H), 0.98-0.91 (m, 3H), 0.84-0.76 (m, 3H) |
| 24 | A | A | 1125.8 | ¹H NMR: (400 MHz, DMSO-d₆) δ: 9.03-8.95 (m, 1H), 8.81-8.32 |
| | | | | (m, 1H), 8.15 (s, 2H), 8.07-7.87 (m, 1H), 7.57-7.35 (m, 5H), 7.28- |
| | | | | 7.05 (m, 1H), 7.00-6.85 (m, 2H), 6.33-6.10 (m, 1H), 6.00-5.90 (m, |
| | | | | 1H), 5.00-4.87 (m, 1H), 4.36-4.33 (m, 1H), 4.32-4.24 (m, 1H), |
| | | | | 4.20-4.10 (m, 1H), 3.76-3.69 (m, 1H), 3.67-3.49 (m, 7H), 3.40- |
| | | | | 3.21 (m, 9H), 3.16-2.90 (m, 5H), 2.84-2.66 (m, 6H), 2.46 (s, 3H), |
| | | | | 2.29-2.12 (m, 5H), 2.06-1.92 (m, 7H), 1.90-1.63 (m, 9H), 1.54- |
| | | | | 1.35 (m, 7H), 1.18-1.05 (m, 2H), 1.01-0.91 (m, 3H), 0.87-0.75 (m, |
| | | | | 3H) |
| 25 | A | A | 1139.7 | ¹H NMR: (400 MHz, DMSO-d₆) δ: 9.01-8.96 (m, 1H), 8.39 (d, J = 7.6 |
| | | | | Hz, 1H), 8.16 (s, 2H), 7.95 (d, J = 7.6 Hz, 1H), 7.57 (s, 1H), 7.48-7.41 |
| | | | | (m, 2H), 7.39-7.34 (m, 2H), 7.24 (m, 1H), 6.92-6.85 (m, 2H), 6.10 |
| | | | | (s, 1H), 6.03-5.97 (m, 2H), 4.96-4.86 (m, 1H), 4.63 (s, 1H), 4.50 (s, |
| | | | | 1H), 4.36 (m, 1H), 4.28 (s, 1H), 4.09 (s, 1H), 3.71 (dd, J = 4.4, 10.8 Hz, |
| | | | | 3H), 3.63-3.49 (m, 3H), 3.45-3.43 (m, 2H), 2.97-2.80 (m, 6H), |
| | | | | 2.78-2.62 (m, 5H), 2.45 (s, 4H), 2.13 (s, 6H), 2.07-1.87 (m, 8H), |
| | | | | 1.77 (d, J = 12.0 Hz, 6H), 1.73-1.54 (m, 8H), 1.47-1.31 (m, 6H), 1.15- |
| | | | | 1.04 (m, 2H), 0.98-0.92 (m, 3H), 0.82-0.76 (m, 3H) |
| 26 | B | A | 1035.7 | |
| 27 | A | A | 1143.7 | ¹H NMR: (400 MHz, DMSO-d₆) δ: 14.43-14.03 (m, 1H), 9.11-8.67 |
| | | | | (m, 1H), 8.41 (d, J = 8.0 Hz, 1H), 7.97 (dd, J = 1.2, 8.0 Hz, 1H), 7.54 |
| | | | | (s, 1H), 7.44 (d, J = 8.4 Hz, 2H), 7.40-7.34 (m, 2H), 7.28-7.21 (m, |
| | | | | 1H), 6.94-6.83 (m, 2H), 6.11 (s, 1H), 5.99-5.84 (m, 2H), 5.38-5.00 |
| | | | | (m, 1H), 4.92 (quin, J = 7.2 Hz, 1H), 4.37 (t, J = 8.0 Hz, 1H), 4.29 (br |
| | | | | s, 1H), 4.09 (br t, J = 4.4 Hz, 1H), 3.72 (br dd, J = 4.4, 10.4 Hz, 1H), |
| | | | | 3.65-3.52 (m, 3H), 3.42 (br d, J = 9.2 Hz, 1H), 3.30-3.18 (m, 5H), |
| | | | | 2.96 (br d, J = 10.4 Hz, 2H), 2.84-2.58 (m, 8H), 2.46 (s, 4H), 2.23 |
| | | | | (td, J = 6.8, 9.6 Hz, 1H), 2.17-2.06 (m, 4H), 2.06-1.50 (m, 21H), |
| | | | | 1.49-1.29 (m, 5H), 1.18-1.03 (m, 2H), 1.01-0.91 (m, 3H), 0.86- |
| | | | | 0.75 (m, 3H) |
| 28 | A | A | 1055.7 | |
| 29 | A | A | 1091.7 | |
| 30 | A | A | 1020.7 | |
| 31 | A | A | 1162.8 | |
| 32 | A | A | 1188.8 | |
| 33 | C | A | 1171.8 | |
| 34 | A | A | 1030.6 | ¹H NMR (400 MHz, MeOD-d₄) δ: 8.50-8.38 (m, 3H), 7.86-7.79 |
| | | | | (m, 2H), 7.60 (s, 1H), 7.51-7.37 (m, 5H), 7.31-7.24 (m, 1H), 6.99- |
| | | | | 6.91 (m, 2H), 6.65 (dd, J = 1.6, 6.0 Hz, 1H), 6.36 (d, J = 2.0 Hz, 1H), |
| | | | | 6.28-6.23 (m, 1H), 6.13-6.03 (m, 1H), 5.36-5.30 (m, 1H), 5.22- |
| | | | | 5.04 (m, 6H), 4.60 (s, 3H), 4.57-4.43 (m, 1H), 3.89-3.82 (m, 4H), |
| | | | | 3.76-3.68 (m, 1H), 3.65-3.61 (m, 4H), 3.53-3.47 (m, 2H), 3.18- |
| | | | | 3.13 (m, 1H), 2.79-2.65 (m, 4H), 2.28-2.12 (m, 1H), 2.02-1.91 (m, |
| | | | | 1H), 1.60-1.48 (m, 3H), 1.08 (d, J = 6.4 Hz, 3H), 0.95-0.90 (m, 3H) |
| 35 | A | A | 1097.7 | ¹H NMR: (400 MHz, DMSO-d₆) δ: 14.22 (br s, 1H), 8.98 (s, 1H), |
| | | | | 8.39 (d, J = 7.6 Hz, 1H), 7.94 (d, J = 7.7 Hz, 1H), 7.53-7.29 (m, 5H), |
| | | | | 7.23 (t, J = 7.2 Hz, 1H), 6.88 (d, J = 8.0 Hz, 2H), 6.09 (s, 1H), 5.87 (s, |
| | | | | 2H), 5.09 (br d, J = 3.2 Hz, 1H), 4.90 (br t, J = 7.2 Hz, 1H), 4.35 (t, |
| | | | | J = 7.6 Hz, 1H), 4.27 (br s, 1H), 3.80-3.65 (m, 2H), 3.62-3.51 (m, |
| | | | | 3H), 3.51-3.39 (m, 3H), 3.26-3.18 (m, 3H), 2.96-2.79 (m, 3H), |
| | | | | 2.75-2.56 (m, 5H), 2.45 (s, 3H), 2.33-2.14 (m, 6H), 2.07-1.97 (m, |
| | | | | 3H), 1.93-1.72 (m, 8H), 1.72-1.40 (m, 11H), 1.37 (d, J = 7.2 Hz, |
| | | | | 3H), 1.16-1.01 (m, 2H), 1.00-0.90 (m, 3H), 0.84-0.72 (m, 3H) |
| 36 | A | A | 1006.6 | ¹H NMR: (400 MHz, MeOD-d₄) δ: 8.89 (s, 1H), 8.41 (s, 1H), 8.12 (s, |
| | | | | 1H), 8.10 (s, 1H), 7.88 (dd, J = 1.6, 8.4 Hz, 1H), 7.50-7.35 (m, 4H), |
| | | | | 7.28 (t, J = 7.2 Hz, 1H), 6.99-6.94 (m, 2H), 6.10 (s, 1H), 5.41-5.35 |
| | | | | (m, 1H), 5.08-5.02 (m, 1H), 4.80-4.55(m, 1H), 4.53 (t, J = 8.0 Hz, |
| | | | | 1H), 4.46 (br s, 1H), 3.86 (dd, J = 4.4, 10.8 Hz, 1H), 3.72-3.45 (m, |
| | | | | 4H), 3.10 (br t, J = 8.0 Hz, 1H), 2.83 (br t, J = 12.4 Hz, 2H), 2.52-2.47 |
| | | | | (m, 3H), 2.46-2.28 (m, 4H), 2.26-2.15 (m, 4H), 2.07-1.85 (m, 3H), |
| | | | | 1.84-1.62 (m, 10H), 1.61-1.51 (m, 3H), 1.47-1.15 (m, 3H), 1.10- |
| | | | | 1.02 (m, 3H), 0.95-0.86 (m, 3H) |
| 37 | D | NA | 1213.7 | |
| 38 | A | A | 1213.7 | |
| 39 | A | A | 1171.8 | |
| 40 | A | A | 1096.7 | |
| 41 | A | A | 1008.6 | |
| 42 | A | A | 1102.7 | ¹H NMR: (400 MHz, DMSO-d₆) δ: 13.29 (br s, 1H), 8.40 (d, J = 7.6 |
| | | | | Hz, 1H), 8.23 (d, J = 13.2 Hz, 2H), 8.01-7.76 (m, 2H), 7.57-7.43 |
| | | | | (m, 3H), 7.41-7.36 (m, 2H), 7.29-7.22 (m, 1H), 7.01-6.86 (m, 4H), |
| | | | | 6.42-6.32 (m, 1H), 6.10 (s, 1H), 5.10 (br d, J = 2.8 Hz, 1H), 4.93 (s, |
| | | | | 1H), 4.36 (t, J = 8.0 Hz, 1H), 4.28 (br s, 1H), 4.19 (br t, J = 7.2 Hz, |
| | | | | 1H), 4.10-4.01 (m, 1H), 3.84 (s, 3H), 3.71 (br dd, J = 4.4, 10.4 Hz, |
| | | | | 1H), 3.63-3.52 (m, 3H), 3.46-3.38 (m, 1H), 3.21 (dt, J = 4.4, 8.4 Hz, |
| | | | | 1H), 2.92-2.60 (m, 7H), 2.30-1.84 (m, 11H), 1.82-1.55 (m, 9H), |
| | | | | 1.49-1.42 (m, 4H), 1.41-1.34 (m, 4H), 1.23-1.02 (m, 5H), 0.99- |
| | | | | 0.87 (m, 3H), 0.85-0.75 (m, 3H) |
| 43 | A | A | 1174.7 | |
| 44 | A | A | 1151.8 | |
| 45 | A | A | 1046.7 | ¹H NMR: (400 MHz, Methanol-d₄) δ: 9.46-9.02 (m, 1H), 7.64-7.53 |
| | | | | (m, 2H), 7.52-7.39 (m, 5H), 7.09-7.01 (m, 2H), 6.15-6.06 (m, 1H), |
| | | | | 5.14-4.99 (m, 2H), 4.51 (t, J = 8.2 Hz, 1H), 4.44 (s, 1H), 4.40-4.33 (m, |
| | | | | 1H), 3.85 (dd, J = 10.8, 4.4 Hz, 1H), 3.80-3.67 (m, 7H), 3.67-3.58 (m, |
| | | | | 3H), 3.50-3.44 (m, 1H), 3.46-3.35 (m, 4H), 3.29-3.19 (m, 2H), 3.10- |
| | | | | 2.98 (m, 3H), 2.91 (t, J = 12.0 Hz, 2H), 2.57-2.50 (m, 3H), 2.46-2.39 |
| | | | | (m, 4H), 2.26-2.14 (m, 2H), 2.10-2.03 (m, 2H), 2.01-1.91 (m, 2H), |
| | | | | 1.84 (d, J = 12.4 Hz, 2H), 1.62-1.49 (m, 3H), 1.45-1.27 (m, 3H), 1.05 |
| | | | | (d, J = 6.4 Hz, 3H), 0.94-0.87 (m, 3H) |
| 46 | D | NA | 1058.6 | |
| 47 | B | A | 1125.8 | ¹H NMR: (400 MHz, DMSO-d₆) δ: 14.41-14.08 (m, 1H), 9.09-8.88 |
| | | | | (m, 1H), 8.39 (d, J = 7.6 Hz, 1H), 8.05-7.85 (m, 1H), 7.64-7.50 (m, |
| | | | | 1H), 7.48-7.41 (m, 2H), 7.40-7.33 (m, 2H), 7.24 (t, J = 7.6 Hz, 1H), |
| | | | | 6.97-6.80 (m, 2H), 6.14-5.93 (m, 1H), 5.91-5.80 (m, 2H), 5.10 (br |
| | | | | s, 1H), 4.91 (quin, J = 7.2 Hz, 1H), 4.36 (t, J = 7.6 Hz, 1H), 4.28 (br s, |
| | | | | 1H), 4.12-4.01 (m, 1H), 3.71 (br dd, J = 4.4, 10.4 Hz, 1H), 3.65- |
| | | | | 3.53 (m, 3H), 3.41 (br d, J = 9.6 Hz, 1H), 3.34 (br s, 1H), 3.26-3.17 |
| | | | | (m, 1H), 3.10 (br s, 2H), 2.81 (br d, J = 10.8 Hz, 1H), 2.77-2.61 (m, |
| | | | | 6H), 2.45 (s, 4H), 2.43-2.29 (m, 4H), 2.28-2.20 (m, 2H), 2.17 (br s, |
| | | | | 3H), 2.13-2.05 (m, 5H), 2.05-1.86 (m, 7H), 1.85-1.59 (m, 10H), |
| | | | | 1.47-1.32 (m, 5H), 1.18-1.02 (m, 2H), 0.99-0.91 (m, 3H), 0.84- |
| | | | | 0.76 (m, 3H) |
| 48 | C | A | 1058.6 | |
| 49 | A | A | 1069.6 | ¹H NMR: (400 MHz, CD₃OD) δ = 8.87 (s, 1H), 8.19 (s, 1H), 7.83- |
| | | | | 7.70 (m, 2H), 7.51-7.35 (m, 5H), 7.24 (t, J = 7.2 Hz, 1H), 6.94-6.86 |
| | | | | (m, 2H), 6.60 (d, J = 6.0 Hz, 1H), 6.29 (s, 1H), 6.12-6.03 (m, 1H), |
| | | | | 5.25-4.97 (m, 3H), 4.60-4.37 (m, 4H), 3.89-3.44 (m, 6H), 3.42-3.35 |
| | | | | (m, 2H), 3.16-3.09 (m, 2H), 3.05-2.52 (m, 7H), 2.51-2.45 (m, 3H), |
| | | | | 2.43-2.12 (m, 7H), 2.08-1.77 (m, 5H), 1.62-1.48 (m, 3H), 1.39-1.22 |
| | | | | (m, 2H), 1.05 (d, J = 6.4 Hz, 3H), 0.95-0.85 (m, 3H) |
| 50 | A | A | 1069.6 | ¹H NMR: (400 MHz, CD₃OD) δ = 8.87 (s, 1H), 8.19 (s, 1H), 7.84- |
| | | | | 7.68 (m, 2H), 7.51-7.35 (m, 5H), 7.28-7.20 (m, 1H), 6.94-6.86 (m, |
| | | | | 2H), 6.63-6.56 (m, 1H), 6.29 (s, 1H), 6.13-6.02 (m, 1H), 5.25-4.98 (m, |
| | | | | 3H), 4.59-4.37 (m, 4H), 3.91-3.34 (m, 8H), 3.16-3.09 (m, 2H), 3.07- |
| | | | | 2.53 (m, 7H), 2.50-2.44 (m, 3H), 2.43-2.10 (m, 7H), 2.07-1.77 (m, |
| | | | | 5H), 1.62-1.48 (m, 3H), 1.39-1.22 (m, 2H), 1.05 (d, J = 6.4 Hz, 3H), |
| | | | | 0.94-0.84 (m, 3H) |
| 51 | A | A | 1038.6 | ¹H NMR: (400 MHz, MeOD-d₄) δ: 8.24 (s, 1H), 8.14 (d, J = 5.6 Hz, |
| | | | | 1H), 7.84 (dd, J = 1.6, 8.0 Hz, 1H), 7.48 (d, J = 2.0 Hz, 1H), 7.46- |
| | | | | 7.38 (m, 4H), 7.32-7.24 (m, 2H), 7.04 (s, 1H), 6.99-6.93 (m, 2H), |
| | | | | 6.76-6.63 (m, 1H), 6.34 (d, J = 2.0 Hz, 1H), 6.11-6.03 (m, 1H), 5.31- |
| | | | | 5.23 (m, 1H), 5.05 (q, J = 6.8 Hz, 1H), 4.52 (t, J = 8.0 Hz, 1H), 4.46- |
| | | | | 4.36 (m, 2H), 3.88-3.80 (m, 4H), 3.72-3.57 (m, 4H), 3.46-3.34 |
| | | | | (m, 1H), 2.90-2.75 (m, 4H), 2.52-2.34 (m, 5H), 2.26-2.09 (m, 5H), |
| | | | | 2.00-1.86 (m, 3H), 1.85-1.71 (m, 3H), 1.66-1.57 (m, 2H), 1.53 (d, |
| | | | | J = 6.8 Hz, 3H), 1.30-1.19 (m, 2H), 1.05 (d, J = 6.8 Hz, 3H), 0.92- |
| | | | | 0.86 (m, 3H) |
| 52 | A | A | 968.6 | ¹H NMR: (400 MHz, MeOD-d₄) δ 8.24 (s, 1H), 8.14 (d, J = 5.6 Hz, |
| | | | | 1H), 7.90-7.82 (m, 1H), 7.53-7.38 (m, 5H), 7.32-7.20 (m, 2H), |
| | | | | 7.04 (s, 1H), 7.01-6.93 (m, 2H), 6.76-6.60 (m, 1H), 6.34 (d, J = 2.0 |
| | | | | Hz, 1H), 6.17-6.00 (m, 1H), 5.05 (q, J = 6.8 Hz, 2H), 4.60-4.42 (m, |
| | | | | 2H), 3.92-3.77 (m, 4H), 3.74-3.56 (m, 4H), 2.92-2.76 (m, 4H), |
| | | | | 2.43-2.26 (m, 5H), 2.22-2.04 (m, 3H), 2.01-1.91 (m, 1H), 1.89- |
| | | | | 1.75 (m, 5H), 1.61-1.47 (m, 3H), 1.32-1.24 (m, 2H), 1.05 (d, J = 6.8 |
| | | | | Hz, 3H), 0.95-0.86 (m, 3H) |
| 53 | B | A | 1087.6 | ¹H NMR: (400 MHz, MeOD) δ: 8.87 (s, 1H), 7.80-7.70 (m, 2H), 7.50- |
| | | | | 7.35 (m, 5H), 7.25-7.20 (m, 1H), 6.90-6.85 (m, 2H), 6.60-6.55 (m, |
| | | | | 1H), 6.30-6.25 (m, 1H), 6.10 (s, 1H), 5.30-5.20 (m, 1H), 5.10-5.00 (m, |
| | | | | 1H), 4.70-4.35 (m, 5H), 3.90-3.80 (m, 1H), 3.75-3.55 (m, 4H), 3.40- |
| | | | | 3.35 (m, 1H), 3.15-3.10 (m, 2H), 3.00-2.80 (m, 3H), 2.70-2.60 (m, |
| | | | | 2H), 2.50-2.40 (m, 4H), 2.40-2.30 (m, 3H), 2.25-2.05 (m, 6H), 2.00- |
| | | | | 1.90 (m, 2H), 1.85-1.70 (m, 3H), 1.60-1.50 (m, 3H), 1.30-1.20 (m, |
| | | | | 2H), 1.04 (d, J = 6.4 Hz, 3H), 0.88 (d, J = 6.4 Hz, 3H) |
| 54 | B | A | 1087.6 | ¹H NMR: (400 MHz, MeOD) δ: 8.87 (s, 1H), 7.85-7.70 (m, 2H), 7.50- |
| | | | | 7.35 (m, 5H), 7.25-7.20 (m, 1H), 6.90-6.85 (m, 2H), 6.60-6.55 (m, |
| | | | | 1H), 6.30-6.25 (m, 1H), 6.10 (s, 1H), 5.30-5.20 (m, 1H), 5.10-5.00 (m, |
| | | | | 1H), 4.70-4.35 (m, 5H), 3.90-3.80 (m, 1H), 3.75-3.55 (m, 4H), 3.40- |
| | | | | 3.35 (m, 1H), 3.15-3.10 (m, 2H), 3.00-2.80 (m, 3H), 2.70-2.60 (m, |
| | | | | 2H), 2.50-2.40 (m, 4H), 2.40-2.30 (m, 3H), 2.25-2.05 (m, 6H), 2.00- |
| | | | | 1.90 (m, 2H), 1.85-1.70 (m, 3H), 1.60-1.50 (m, 3H), 1.30-1.20 (m, |
| | | | | 2H), 1.04 (d, J = 6.4 Hz, 3H), 0.88 (d, J = 6.4 Hz, 3H) |
| 55 | A | A | 1108.7 | |
| 56 | A | A | 1072.7 | |
| 57 | A | A | 1115.7 | |
| 58 | A | A | 1109.7 | |
| 59 | C | A | 1069.6 | ¹H NMR: (400 MHz, Methanol-d₄) δ: 8.87 (s, 1H), 8.30-8.20 (m, 1H), |
| | | | | 7.85-7.70 (m, 2H), 7.50-7.35 (m, 5H), 7.30-7.25 (m, 1H), 6.95-6.90 |
| | | | | (m, 2H), 6.60-6.55 (m, 1H), 6.30-6.25 (m, 1H), 6.15-6.05 (m, 1H), |
| | | | | 5.10-4.95 (m, 2H), 4.70-4.35 (m, 5H), 3.90-3.80 (m, 1H), 3.70-3.60 |
| | | | | (m, 4H), 3.50-3.45 (m, 1H), 3.15-3.10 (m, 3H), 2.90-2.80 (m, 3H), |
| | | | | 2.60-2.30 (m, 8H), 2.25-2.10 (m, 6H), 2.05-1.95 (m, 1H), 1.90-1.70 |
| | | | | (m, 4H), 1.60-1.45 (m, 3H), 1.35-1.15 (m, 3H), 1.10-1.00 (m, 3H), |
| | | | | 0.95-0.85 (m, 3H) |
| 60 | A | A | 1069.6 | ¹H NMR: (400 MHz, Methanol-d₄) δ: 8.87 (s, 1H), 8.30-8.25 (m, 1H), |
| | | | | 7.80-7.70 (m, 2H), 7.50-7.35 (m, 5H), 7.25-7.20 (m, 1H), 6.95-6.85 |
| | | | | (m, 2H), 6.60-6.55 (m, 1H), 6.30-6.25 (m, 1H), 6.15-6.05 (m, 1H), |
| | | | | 5.10-4.95 (m, 2H), 4.70-4.45 (m, 5H), 3.90-3.80 (m, 1H), 3.75-3.60 |
| | | | | (m, 4H), 3.50-3.40 (m, 1H), 3.15-3.10 (m, 3H), 2.95-2.80 (m, 3H), |
| | | | | 2.55-2.35 (m, 8H), 2.30-2.15 (m, 6H), 2.00-1.90 (m, 1H), 1.90-1.70 |
| | | | | (m, 4H), 1.60-1.50 (m, 3H), 1.45-1.15 (m, 3H), 1.10-1.00 (m, 3H), |
| | | | | 0.95-0.85 (m, 3H) |
| 61 | A | A | 1111.7 | ¹H NMR: (400 MHz, DMSO-d₆) δ: 8.99 (s, 1H), 8.39 (d, J = 7.6 Hz, |
| | | | | 1H), 8.15 (s, 2H), 7.93 (d, J = 8.0 Hz, 1H), 7.50-7.42 (m, 3H), 7.40- |
| | | | | 7.34 (m, 2H), 7.25 (t, J = 7.6 Hz, 1H), 6.98-6.80 (m, 2H), 6.23-5.96 |
| | | | | (m, 1H), 5.90 (s, 2H), 5.26-4.61 (m, 3H), 4.36 (t, J = 7.6 Hz, 1H), |
| | | | | 4.29 (br s, 1H), 3.71 (br dd, J = 4.4, 10.4 Hz, 3H), 3.54 (br s, 6H), |
| | | | | 2.96 (br t, J = 11.2 Hz, 3H), 2.80-2.65 (m, 3H), 2.63-2.56 (m, 1H), |
| | | | | 2.46 (s, 3H), 2.40-2.11 (m, 10H), 2.08-1.72 (m, 11H), 1.67 (br s, |
| | | | | 5H), 1.60-1.48 (m, 5H), 1.46-1.33 (m, 4H), 1.08 (br dd, J = 6.4, |
| | | | | 13.6 Hz, 2H), 1.01-0.90 (m, 3H), 0.85-0.72 (m, 3H) |
| 62 | A | A | 1111.7 | ¹H NMR: (400 MHz, DMSO-d₆) δ: 8.99 (s, 1H), 8.40 (d, J = 7.6 Hz, |
| | | | | 1H), 8.19 (s, 1H), 7.95 (br dd, J = 8.0, 12.4 Hz, 1H), 7.57 (d, J = 9.2 Hz, |
| | | | | 1H), 7.49-7.42 (m, 2H), 7.40-7.34 (m, 2H), 7.25 (t, J = 7.6 Hz, 1H), |
| | | | | 6.89 (dd, J = 2.8, 8.0 Hz, 2H), 6.11 (s, 1H), 6.05-5.95 (m, 2H), 4.96- |
| | | | | 4.87 (m, 1H), 4.69-4.51 (m, 2H), 4.36 (t, J = 7.6 Hz, 1H), 4.31-4.16 |
| | | | | (m, 2H), 3.86 (br d, J = 9.6 Hz, 1H), 3.76-3.68 (m, 1H), 3.66-3.54 |
| | | | | (m, 12H), 3.04-2.87 (m, 2H), 2.79-2.66 (m, 5H), 2.46 (s, 3H), 2.37- |
| | | | | 2.06 (m, 6H), 2.01-1.62 (m, 12H), 1.59-1.42 (m, 6H), 1.38 (d, J-7.2 |
| | | | | Hz, 3H), 1.09 (br d, J = 9.2 Hz, 2H), 0.99-0.91 (m, 3H), 0.83-0.76 |
| | | | | (m, 3H) |
| 63 | A | A | 978.6 | ¹H NMR: (400 MHz, DMSO-d₆) δ: 13.3 (s, 1H), 8.98 (s, 1H), 8.40 (d, |
| | | | | J = 7.6 Hz, 1H), 8.32 (s, 1H), 8.22 (s, 1H), 7.91-7.85 (m, 2H), 7.47- |
| | | | | 7.41 (m, 2H), 7.39-7.34 (m, 2H), 7.28-7.22 (m, 1H), 6.97 (s, 2H), |
| | | | | 6.94-6.87 (m, 2H), 6.10 (s, 1H), 5.10 (d, J = 4.0 Hz, 1H), 4.95-4.89 |
| | | | | (m, 2H), 4.36 (t, J = 7.6 Hz, 1H), 4.28 (s, 1H), 3.75-3.68 (m, 1H), |
| | | | | 3.64-3.58 (m, 2H), 3.56 (d, J = 10.0 Hz, 1H), 3.45-3.40 (m, 1H), |
| | | | | 2.77-2.68 (m, 2H), 2.46 (s, 3H), 2.35-2.30 (m, 3H), 2.25-2.15 (m, |
| | | | | 5H), 2.13-2.07 (m, 2H), 2.05-1.97 (m, 1H), 1.84-1.76 (m, 1H), |
| | | | | 1.73-1.56 (m, 8H), 1.38 (d, J = 7.2 Hz, 3H), 1.15-1.04 (m, 2H), 0.95 |
| | | | | (d, J = 6.4 Hz, 3H), 0.79 (d, J = 6.4 Hz, 3H) |
| 64 | B | A | 923.5 | |
| 65 | A | A | 976.6 | |
| 66 | B | A | 955.1 | ¹H NMR (400 MHz, DMSO-d₆): δ 8.49 (d, J = 7.2 Hz, 1H), 8.21 (s, |
| | | | | 2H), 7.91 (d, J = 7.2 Hz, 1H), 7.81-7.75 (m, 3H), 7.51-7.44 (m, |
| | | | | 3H), 7.24-7.19 (m, 1H), 6.88-6.82 (m, 2H), 6.56-6.52 (m, 1H), |
| | | | | 6.15 (s, 1H), 6.08 (s, 1H), 5.96 (s, 2H), 4.94-4.90 (m, 1H), 4.50- |
| | | | | 4.48 (m, 2H), 4.37-4.31 (m, 1H), 4.29-4.20 (m, 5H), 3.67 (m, 3H), |
| | | | | 3.64-3.62 (m, 2H), 3.43-3.42 (m, 2H), 3.26-3.22 (m, 3H), 3.02 (s, |
| | | | | 2H), 2.68-2.62 (m, 3H), 2.47-2.44 (m, 4H), 2.21-2.10 (m, 3H), |
| | | | | 2.08-1.91 (m, 3H), 1.72 (s, 1H), 1.36-1.34 (m, 3H), 0.95-0.91 (m, |
| | | | | 3H), 0.85-0.72 (m, 3H) |
| 67 | C | A | 1059.7 | |
| 68 | A | A | 1059.7 | |
| 69 | A | A | 1121.8 | ¹H NMR: (400 MHz, DMSO-d₆) δ: 8.99 (s, 1H), 8.39 (d, J = 7.6 Hz, |
| | | | | 1H), 8.28 (s, 1H), 8.14 (s, 2H), 8.06-7.97 (m, 1H), 7.86 (s, 1H), 7.63 |
| | | | | (s, 1H), 7.47-7.41 (m, 3H), 7.40-7.33 (m, 3H), 7.25 (m, 1H), 7.03 |
| | | | | (d, J = 16.0 Hz, 1H), 6.91 (d, J = 8.0 Hz, 2H), 6.79 (m, 2H), 6.20-6.09 |
| | | | | (s, 1H), 5.10 (s, 1H), 5.02-4.85 (m, 2H), 4.35-4.26 (m, 3H), 4.25- |
| | | | | 4.17 (m, 1H), 4.14-3.96 (m, 2H), 3.83-3.53 (m, 5H), 3.19-3.02 (m, |
| | | | | 3H), 3.00-2.92 (m, 3H), 2.87-2.79 (m, 3H), 2.75 (m, 4H), 2.30-2.14 |
| | | | | (m, 5H), 2.07-1.98 (m, 4H), 1.84-1.76 (m, 5H), 1.71 (m, 4H), 1.45 |
| | | | | (d, J = 6.8 Hz, 3H), 1.37 (d, J = 6.8 Hz, 3H), 1.22-1.08 (m, 4H), 1.01- |
| | | | | 0.88 (m, 3H), 0.85-0.71 (m, 3H) |
| 70 | A | A | 1095.8 | ¹H NMR: (400 MHz, DMSO-d₆) δ: 14.23 ( s, 1H), 8.98 (s, 1H), 8.39 |
| | | | | (d, J = 7.6 Hz, 1H), 7.98-7.91 (m, 1H), 7.49 (s, 1H), 7.47-7.41 (m, |
| | | | | 2H), 7.39-7.33 (m, 2H), 7.27-7.21 (m, 1H), 6.91-6.84 (m, 2H), |
| | | | | 6.09 (s, 1H), 5.87 (s, 2H), 5.10 (s, 1H), 4.91 (m, 1H), 4.36 (m, 1H), |
| | | | | 4.28 (s, 1H), 3.71 (dd, J = 4.4, 10.4 Hz, 1H), 3.64-3.51 (m, 3H), 3.45- |
| | | | | 3.37 (m, 2H), 3.30-3.19 (m, 5H), 2.95-2.79 (m, 3H), 2.74-2.62 (m, |
| | | | | 3H), 2.58-2.51 (m, 1H), 2.45 (s, 3H), 2.29-2.12 (m, 6H), 2.06-1.98 |
| | | | | (m, 3H), 1.87 (s, 6H), 1.72-1.55 (m, 7H), 1.53-1.33 (m, 12H), 1.07 |
| | | | | (d, J = 11.8 Hz, 2H), 0.98-0.91 (m, 3H), 0.84-0.76 (m, 3H) |
| 71 | A | A | 1111.7 | ¹H NMR: (400 MHz, DMSO-d₆) δ: 8.98 (s, 1H), 8.39 (d, J = 7.6 Hz, |
| | | | | 1H), 8.23-8.16 (m, 3H), 7.92 (br d, J = 8.0 Hz, 1H), 7.50-7.39 (m, |
| | | | | 3H), 7.39-7.32 (m, 2H), 7.27-7.19 (m, 1H), 6.93-6.83 (m, 2H), |
| | | | | 6.12-5.96 (m, 1H), 5.88 (s, 2H), 4.97-4.59 (m, 2H), 4.35 (br t, J = |
| | | | | 8.0 Hz, 2H), 4.27 (br d, J = 3.6 Hz, 2H), 3.70 (br dd, J = 4.8, 10.4 Hz, |
| | | | | 3H), 3.61-3.52 (m, 7H), 2.96 (br dd, J = 11.2, 16.4 Hz, 3H), 2.77- |
| | | | | 2.68 (m, 2H), 2.45 (s, 3H), 2.37 (br d, J = 11.2 Hz, 3H), 2.29-2.14 |
| | | | | (m, 5H), 2.13-1.97 (m, 4H), 1.96-1.83 (m, 6H), 1.80-1.74 (m, 1H), |
| | | | | 1.72-1.58 (m, 6H), 1.54-1.42 (m, 6H), 1.37 (d, J = 7.2 Hz, 3H), 1.11- |
| | | | | 1.03 (m, 2H), 0.98-0.89 (m, 3H), 0.84-0.73 (m, 3H) |
| 72 | D | NA | 1191.6 | |
| 73 | A | A | 1111.7 | |
| 74 | A | A | 1095.7 | ¹H NMR: (400 MHz, MeOD-d₄) δ: 8.86 (s, 1H), 8.45 (s, 1H), 8.20 (s, |
| | | | | 1H), 7.91 (dd, J = 1.6, 8.4 Hz, 1H), 7.76 (s, 1H), 7.48-7.33 (m, 4H), |
| | | | | 7.31-7.22 (m, 1H), 7.02-6.89 (m, 2H), 6.12-5.96 (m, 1H), 5.02 (d, |
| | | | | J = 7.2 Hz, 1H), 4.50 (t, J = 8.0 Hz, 1H), 4.42 (br d, J = 2.0 Hz, 1H), |
| | | | | 4.17 (td, J = 3.6, 6.8 Hz, 1H), 3.88-3.71 (m, 1H), 3.68-3.42 (m, 4H), |
| | | | | 3.33 (br s, 1H), 3.04-2.69 (m, 8H), 2.47 (s, 3H), 2.42-2.29 (m, 1H), |
| | | | | 2.26-2.02 (m, 9H), 1.95 (ddd, J = 4.8, 8.8, 13.2 Hz, 1H), 1.87-1.63 |
| | | | | (m, 9H), 1.60-1.44 (m, 6H), 1.33 (d, J = 7.2 Hz, 4H), 1.29-1.14 (m, |
| | | | | 3H), 1.04 (d, J = 6.4 Hz, 3H), 0.92-0.83 (m, 3H) |
| 75 | A | A | 1095.7 | ¹H NMR: (400 MHz, MeOD-d₄) δ: 8.87 (s, 1H), 8.46 (s, 1H), 8.20 (s, |
| | | | | 1H), 7.92 (dd, J = 1.6, 8.4 Hz, 1H), 7.76 (s, 1H), 7.48-7.34 (m, 4H), |
| | | | | 7.27 (dt, J = 1.6, 7.6 Hz, 1H), 7.00-6.90 (m, 2H), 6.11-5.92 (m, 1H), |
| | | | | 5.03 (d, J = 7.2 Hz, 1H), 4.51 (t, J = 8.4 Hz, 1H), 4.46-4.35 (m, 1H), |
| | | | | 4.17 (td, J = 3.2, 6.8 Hz, 1H), 3.94-3.72 (m, 1H), 3.70-3.43 (m, 4H), |
| | | | | 3.34 (br s, 1H), 3.04-2.66 (m, 8H), 2.47 (s, 3H), 2.43-2.26 (m, 1H), |
| | | | | 2.23-2.01 (m, 9H), 1.95 (ddd, J = 4.8, 8.8, 13.2 Hz, 1H), 1.89-1.61 |
| | | | | (m, 9H), 1.59-1.45 (m, 6H), 1.33 (d, J = 7.2 Hz, 4H), 1.28-1.14 (m, |
| | | | | 3H), 1.04 (d, J = 6.8 Hz, 3H), 0.95-0.81 (m, 3H) |
| 76 | B | A | 1139.8 | |
| 77 | A | A | 1086.7 | ¹H NMR: (400 MHz, MeOD-d₄) δ: 8.94 (s, 1H), 7.78 (d, J = 6.0 Hz, |
| | | | | 1H), 7.47-7.45 (m, 4H), 7.45-7.44 (m, 1H), 7.09 (s, 1H), 6.93-6.91 |
| | | | | (m, 2H), 6.11 (s, 1H), 5.06-5.04 (m, 1H), 4.60-4.52 (m, 2H), 4.50- |
| | | | | 4.40 (m, 1H), 4.42-4.32 (m, 2H), 4.36-4.35 (m, 1H), 3.85-3.84 (m, |
| | | | | 2H), 3.72-3.54 (m, 9H), 3.33-3.32 (m, 2H), 2.87-2.67 (m, 4H), |
| | | | | 2.50-2.46 (m, 5H), 2.44-2.32 (m, 5H), 2.32-2.17 (m, 5H), 1.98- |
| | | | | 1.94 (m, 5H), 1.96-1.94 (m, 3H), 1.94-1.92 (m, 4H), 1.55 (d, |
| | | | | J = 7.2 Hz, 3H), 1.07-1.06 (m, 3H), 0.91-0.89 (m, 3H) |
| 78 | B | A | 1047.7 | |
| 79 | D | NA | 1052.6 | |
| 80 | A | A | 1052.6 | |
| 81 | A | A | 993.7 | ¹H NMR (400 MHz, CD₃OD): δ 8.88 (s, 1H), 7.80 (d, J = 7.2 Hz, 1H), |
| | | | | 7.56 (s, 1H), 7.49-7.36 (m, 4H), 7.28-7.23 (m, 1H), 7.00-6.87 (m, |
| | | | | 2H), 6.08-5.97 (m, 1H), 5.13-4.99 (m, 2H), 4.75-4.70 (m, 2H), |
| | | | | 4.51 (t, J = 8.4 Hz, 1H), 4.47-4.35 (m, 4H), 3.84 (dd, J₁ = 10.8, J₂ = |
| | | | | 4.0 Hz, 1H), 3.79-3.65 (m, 5H), 3.62 (d, J = 11.2 Hz, 1H), 3.51- |
| | | | | 3.44 (m, 1H), 3.16-3.14 (m, 4H), 2.99-2.91 (m, 4H), 2.54 (s, 1H), |
| | | | | 2.48 (s, 3H), 2.43-2.33 (m, 5H), 2.24-2.12 (m, 1H), 2.03-1.87 (m, |
| | | | | 3H), 1.68 (d, J = 7.4 Hz, 2H), 1.61-1.48 (m, 3H), 1.06 (d, J = 6.4 Hz, |
| | | | | 3H), 0.96-0.83 (m, 3H) |
| 82 | D | NA | 1135.6 | |
| 83 | D | NA | 1135.6 | |
| 84 | A | A | 1120.8 | ¹H NMR: (400 MHz, DMSO-d₆) δ: 8.41 (d, J = 7.6 Hz, 1H), 8.26 (s, |
| | | | | 1H), 7.97-7.88 (m, 2H), 7.52-7.37 (m, 5H), 7.27 (t, J = 7.2 Hz, 1H), |
| | | | | 7.01 (br s, 2H), 6.98-6.87 (m, 2H), 6.39-6.36 (m, 1H), 6.17 (br s, |
| | | | | 1H), 5.13 (d, J = 3.6 Hz, 1H), 4.94 (t, J = 7.2 Hz, 1H), 4.36 (t, J = 8.0 Hz, |
| | | | | 1H), 4.33-4.12 (m, 3H), 3.85 (s, 3H), 3.75-3.55 (m, 4H), 3.48-3.43 |
| | | | | (m, 1H), 3.06-2.87(m, 4H), 2.82-2.72 (m, 3H), 2.55-2.52 (m, 3H), |
| | | | | 2.30-2.13 (m, 4H), 2.06-1.63 (m, 14H), 1.47 (br d, J = 6.0 Hz, 5H), |
| | | | | 1.42-1.37 (m, 3H), 1.32-1.06 (m, 6H), 1.00-0.93 (m, 3H), 0.84- |
| | | | | 0.76 (m, 3H) |
| 85 | D | NA | 1095.7 | ¹H NMR (400 MHz, DMSO-d₆) |
| | | | | δ: 13.19 (s, 1H), 8.98 (s, 1H), 8.40 (d, J = 7.6 Hz, 1H), 8.17 (s, 1H), |
| | | | | 8.03 (d, J = 1.2 Hz, 1H), 7.98-7.94 (m, 1H), 7.46-7.41 (m, 2H), 7.39- |
| | | | | 7.35 (m, 2H), 7.34-7.31 (m, 1H), 7.30-7.25 (m, 1H), 6.96-6.87 |
| | | | | (m, 2H), 6.74 (s, 2H), 6.09 (s, 1H), 5.11 (br, 1H), 4.96-4.86 (m, 1H), |
| | | | | 4.36 (t, J = 7.6 Hz, 1H), 4.28 (s, 1H), 4.10-4.00 (m, 1H), 3.75-3.67 |
| | | | | (m, 1H), 3.63-3.57 (m, 2H), 3.56-3.52 (m, 1H), 3.25-3.14 (m, 2H), |
| | | | | 2.92-2.81 (m, 2H), 2.76-2.70 (m, 2H), 2.69-2.62 (m, 4H), 2.61- |
| | | | | 2.57 (m, 1H), 2.45 (s, 3H), 2.26-2.17 (m, 1H), 2.11-2.04 (m, 4H), |
| | | | | 1.98-1.87 (m, 5H), 1.80-1.63 (m, 7H), 1.57-1.50 (m, 4H), 1.47- |
| | | | | 1.41 (m, 1H), 1.37 (d, J = 6.4 Hz, 3H), 1.31-1.23 (m, 1H), 1.19 (d, |
| | | | | J = 6.4 Hz, 3H), 1.11-1.02 (m, 2H), 0.95 (d, J = 6.4 Hz, 3H), 0.85- |
| | | | | 0.80 (m, 1H), 0.78 (d, J = 6.4 Hz, 3H). |
| 86 | B | A | 1047.7 | ¹H NMR (400 MHz, DMSO-d₆) δ: 14.14 (s, 1 H), 9.03 (s, 1H), 8.50- |
| | | | | 8.41 (m, 1H), 7.92-7.86 (m, 1H), 7.77 (d, J = 6.0 Hz, 1H), 7.70-7.65 |
| | | | | (m, 3H), 7.58-7.53 (m, 2H), 7.48-7.43 (m, 2H), 7.31 (d, J = 8.4 Hz, |
| | | | | 2H), 7.25-7.17 (m, 1H), 6.89-6.77 (m, 2H), 6.55-6.50 (m, 1H), |
| | | | | 6.16 (d, J = 2.0 Hz, 1H), 5.95 (s, 2H), 5.10 (s, 1H), 4.90-4.80 (m, 1H), |
| | | | | 4.64 (d, J = 10.4 Hz, 1H), 4.55-4.44 (m, 4H), 4.42 (s, 2H), 4.35-4.25 |
| | | | | (m, 3H), 4.17 (s, 1H), 3.70-3.65 (m, 2H), 3.64-3.57 (m, 4H), 3.55- |
| | | | | 3.44 (m, 1H), 3.25-3.17 (m, 3H), 2.99 (d, J = 11.6 Hz, 2H), 2.37-2.30 |
| | | | | (m, 1H), 2.20-2.12 (m, 2H), 2.08-2.00 (m, 1H), 1.97-1.89 (m, 2H), |
| | | | | 1.66-1.57 (m, 1H), 1.35 (d, J = 7.2 Hz, 3H), 0.98 (d, J = 6.4 Hz, 3H), |
| | | | | 0.70 (d, J = 6.4 Hz, 3H) |
| 87 | | | 1047.7 | ¹H NMR (400 MHz, DMSO-d₆) δ: 14.09 (s, 1 H), 9.08-8.97 (m, 1H), |
| | | | | 8.42 (br, 1H), 7.95-7.85 (m, 1H), 7.81-7.65 (m, 4H), 7.65-7.55 (m, |
| | | | | 2H), 7.52-7.42 (m, 2H), 7.40-7.30 (m, 2H), 7.25-7.10 (m, 1H), |
| | | | | 6.92-6.75 (m, 2H), 6.55-6.43 (m, 1H), 6.20-6.10 (m, 1H), 6.00- |
| | | | | 5.85 (m, 2H), 5.07 (br, 1H), 4.90 (br, 1H), 4.75-4.61 (m, 1H), 4.58- |
| | | | | 4.50 (m, 1H), 4.47-4.37 (m, 5H), 4.32-4.17 (m, 3H), 3.65-3.50 (m, |
| | | | | 8H), 3.25-3.15 (m, 3H), 3.05-2.90 (m, 2H), 2.35-2.23 (m, 1H), |
| | | | | 2.20-2.07 (m, 2H), 2.03-1.85 (m, 3H), 1.80-1.67 (m, 1H), 1.45- |
| | | | | 1.25 (m, 3H), 1.05-0.85 (m, 3H), 0.75-0.60 (m, 3H) |
| 88 | A | A | 1067.7 | ¹H NMR: (400 MHz, DMSO-d₆) δ: 8.99 (s, 1H), 8.49 (s, 1H), 8.40 (d, |
| | | | | J = 7.6 Hz, 1H), 8.23 (d, J = 6.8 Hz, 2H), 8.03 (dd, J = 1.6, 8.4 Hz, |
| | | | | 1H), 7.51-7.41 (m, 2H), 7.40-7.33 (m, 2H), 7.32-7.23 (m, 1H), |
| | | | | 7.01-6.89 (m, 2H), 6.50 (s, 2H), 6.09 (s, 1H), 4.92 (t, J = 7.2 Hz, |
| | | | | 1H), 4.46 (t, J = 6.8 Hz, 1H), 4.36 (t, J = 8.0 Hz, 1H), 4.28 (br d, J = |
| | | | | 2.4 Hz, 1H), 3.86 (br d, J = 10.8 Hz, 1H), 3.77-3.67 (m, 2H), 3.64- |
| | | | | 3.40 (m, 6H), 3.18 (dt, J = 3.6, 7.2 Hz, 1H), 2.77-2.64 (m, 2H), 2.63- |
| | | | | 2.53 (m, 1H), 2.46 (s, 3H), 2.30-2.14 (m, 4H), 2.13-1.94 (m, 5H), |
| | | | | 1.90-1.71 (m, 4H), 1.69-1.56 (m, 4H), 1.54 (d, J = 6.8 Hz, 3H), 1.50- |
| | | | | 1.42 (m, 3H), 1.38 (br d, J = 7.2 Hz, 7H), 1.14-1.00 (m, 2H), 0.98- |
| | | | | 0.89 (m, 3H), 0.79 (d, J = 6.8 Hz, 3H) |
| 89 | A | A | 1067.7 | ¹H NMR: (400 MHz, DMSO-d₆) δ: 9.03-8.73 (m, 1H), 8.47 (s, 1H), |
| | | | | 8.40 (d, J = 7.6 Hz, 1H), 8.22 (s, 1H), 8.16 (s, 1H), 8.02 (dd, J = 1.6, |
| | | | | 8.4 Hz, 1H), 7.44 (d, J = 8.4 Hz, 2H), 7.41-7.34 (m, 2H), 7.31-7.24 |
| | | | | (m, 1H), 6.96-6.89 (m, 2H), 6.47 (s, 2H), 6.10 (s, 1H), 5.24-4.62 |
| | | | | (m, 2H), 4.54-4.41 (m, 1H), 4.36 (t, J = 7.6 Hz, 1H), 4.28 (br s, 1H), |
| | | | | 3.88-3.67 (m, 3H), 3.65-3.52 (m, 3H), 3.48-3.40 (m, 3H), 3.27- |
| | | | | 3.09 (m, 1H), 2.79-2.53 (m, 5H), 2.46 (s, 3H), 2.29-2.13 (m, 4H), |
| | | | | 2.10-1.95 (m, 3H), 1.95-1.84 (m, 2H), 1.83-1.73 (m, 2H), 1.73- |
| | | | | 1.56 (m, 4H), 1.52 (br s, 3H), 1.46 (br d, J = 6.8 Hz, 6H), 1.38 (d, J = |
| | | | | 7.2 Hz, 3H), 1.13-1.03 (m, 2H), 0.99-0.89 (m, 3H), 0.85-0.73 (m, 3H) |
| 90 | D | NA | 1102.8 | |
| 91 | A | A | 1102.8 | |
| 92 | | | 1057.7 | ¹H NMR: (400 MHz, MeOD-d₆) δ: 8.88 (s, 1 H), 8.46 (s, 1 H), 7.72- |
| | | | | 7.87 (m, 1 H), 7.52 (s, 1 H), 7.36-7.47 (m, 4 H), 7.26 (t, J = 3 Hz, 1 |
| | | | | H), 6.88-6.96 (m, 2 H), 6.02-6.15 (m, 1 H), 4.98-5.10 (m, 1 H), |
| | | | | 4.97-5.11 (m, 1 H), 4.71-4.79 (m, 1 H), 4.63 (br s, 1 H), 4.50 (t, J = 8 |
| | | | | Hz, 1 H), 4.34-4.45 (m, 1 H), 3.94 (br d, J = 10 Hz, 1 H), 3.84 (dd, |
| | | | | J = 10.8, 4.13 Hz, 1 H), 3.77 (br s, 1 H), 3.66-3.74 (m, 4 H), 3.59- |
| | | | | 3.65 (m, 2 H), 3.54 (br s, 1 H), 3.34-3.49 (m, 5 H), 3.02-3.13 (m, 3 |
| | | | | H), 2.86-2.93 (m, 2 H), 2.60-2.81 (m, 6 H), 2.48 (s, 3 H), 2.30- |
| | | | | 2.42 (m, 2 H), 2.02-2.22 (m, δ H), 1.96 (ddd, J = 13.20, 8.76, 4.57 Hz, |
| | | | | 2 H) 1.82 (br d, J = 11.38 Hz, 4 H) 1.50-1.60 (m, 3 H) 1.32 (br d, |
| | | | | J = 9.2 Hz, 2 H), 1.02-1.07 (m, 3 H), 0.85-0.92 (m, 3 H) |
| 93 | D | NA | 1064.7 | ¹H NMR: (400 MHz, DMSO-d₆) δ: 14.14 (s, 1H), 8.35 (d, J = 7.6 Hz, |
| | | | | 1H), 7.92 (dd, J = 1.2, 8.0 Hz, 1H), 7.84-7.71 (m, 3H), 7.56-7.47 |
| | | | | (m, 1H), 7.42 (d, J = 8.4 Hz, 2H), 7.26-7.19 (m, 1H), 6.91-6.80 (m, |
| | | | | 2H), 6.53 (dd, J = 2.0, 6.0 Hz, 1H), 6.14 (d, J = 1.6 Hz, 1H), 6.10- |
| | | | | 6.02 (m, 1H), 5.97 (s, 2H), 5.24-5.14 (m, 1H), 5.12 (d, J = 3.6 Hz, |
| | | | | 1H), 5.06-4.84 (m, 1H), 4.82-4.71 (m, 1H), 4.49 (br s, 2H), 4.40 (t, |
| | | | | J = 7.6 Hz, 1H), 4.31-4.19 (m, 2H), 3.74 (d, J = 8.8 Hz, 1H), 3.59- |
| | | | | 3.51 (m, 1H), 3.51-3.44 (m, 1H), 3.29-3.16 (m, 4H), 3.02 (br d, J = |
| | | | | 11.6 Hz, 2H), 2.85-2.70 (m, 3H), 2.31-2.23 (m, 6H), 2.20-1.93 (m, |
| | | | | 9H), 1.84-1.69 (m, 3H), 1.45-1.28 (m, 5H), 0.98-0.90 (m, 8H), |
| | | | | 0.86-0.73 (m, 4H) |
| 94 | B | A | 1125.8 | ¹H NMR: (400 MHz, DMSO-d₆) δ: 14.23 (br s, 1H), 8.99 (s, 1H), |
| | | | | 8.84-8.33 (m, 1H), 7.91 (d, J = 7.6 Hz, 1H), 7.50-7.31 (m, 5H), 7.29- |
| | | | | 7.16 (m, 1H), 6.95-6.81 (m, 2H), 6.10 (s, 1H), 6.00-5.79 (m, 2H), |
| | | | | 5.11 (d, J = 3.6 Hz, 1H), 4.92 (quin, J = 7.2 Hz, 1H), 4.37 (t, J = 7.6 |
| | | | | Hz, 1H), 4.29 (br s, 1H), 3.85 (br d, J = 9.6 Hz, 1H), 3.79-3.67 (m, |
| | | | | 2H), 3.66-3.53 (m, 4H), 3.52-3.39 (m, 2H), 3.31-3.21 (m, 2H), |
| | | | | 3.09 (br d, J = 10.8 Hz, 1H), 2.86-2.58 (m, 6H), 2.46 (s, 3H), 2.32- |
| | | | | 2.07 (m, 5H), 2.06-1.81 (m, 8H), 1.80-1.53 (m, 8H), 1.52-1.33 (m, |
| | | | | 9H), 1.12-0.90 (m, 11H), 0.87-0.75 (m, 3H) |
| 95 | B | A | 1125.8 | ¹H NMR: (400 MHz, DMSO-d₆) δ: 14.22 (br s, 1H), 8.99 (s, 1H), |
| | | | | 8.83-8.35 (m, 1H), 7.92 (br d, J = 7.6 Hz, 1H), 7.49-7.34 (m, 5H), |
| | | | | 7.29-7.18 (m, 1H), 6.93-6.84 (m, 2H), 6.11 (s, 1H), 5.89 (s, 2H), |
| | | | | 5.16-4.99 (m, 1H), 4.92 (quin, J = 7.2 Hz, 1H), 4.37 (t, J = 7.6 Hz, |
| | | | | 1H), 4.29 (br d, J = 2.0 Hz, 1H), 3.86 (br d, J = 10.0 Hz, 1H), 3.78- |
| | | | | 3.67 (m, 2H), 3.65-3.54 (m, 4H), 3.52-3.38 (m, 4H), 3.16-3.05 (m, |
| | | | | 1H), 2.82 (br d, J = 10.0 Hz, 1H), 2.78-2.63 (m, 5H), 2.46 (s, 3H), |
| | | | | 2.31-2.10 (m, 4H), 2.09-1.85 (m, 7H), 1.84-1.59 (m, 9H), 1.57- |
| | | | | 1.44 (m, 6H), 1.39 (d, J = 7.2 Hz, 3H), 1.18-0.97 (m, 9H), 0.95 (br d, |
| | | | | J = 6.8 Hz, 3H), 0.86-0.76 (m, 3H) |
| 96 | A | A | 1091.7 | ¹H NMR: (400 MHz, DMSO-d₆) δ: 8.98 (s, 1H), 8.24 (d, J = 15.6 Hz, |
| | | | | 2H), 8.16 (s, 2H), 7.92-7.83 (m, 2H), 7.43 (d, J = 8.4 Hz, 2H), 7.38- |
| | | | | 7.35 (m, 2H), 7.28-7.21 (m, 1H), 7.01-6.95 (m, 1H), 7.01-6.95 (m, |
| | | | | 1H), 6.94-6.86 (m, 2H), 6.09 (s, 1H), 4.95-4.86 (m, 1H), 4.47 (d, |
| | | | | J = 6.8 Hz, 1H), 4.35 (d, J = 7.6 Hz, 1H), 4.28 (s, 1H), 3.86 (d, J = 11.2 |
| | | | | Hz, 1H), 3.70 (d, J = 4.8, 10.8 Hz, 1H), 3.66-3.57 (m, 4H), 3.48 (d, |
| | | | | J = 9.6 Hz, 2H), 2.77-2.65 (m, 3H), 2.63-2.52 (m, 3H), 2.45 (s, 4H), |
| | | | | 2.26-2.17 (m, 5H), 2.06-1.96 (m, 1H), 1.79 (d, J = 8.0 Hz, 4H), 1.67 |
| | | | | (d, J = 9.6 Hz, 4H), 1.52 (d, J = 10.0 Hz, 3H), 1.48-1.42 (m, 6H), 1.40- |
| | | | | 1.33 (m, 5H), 1.19-1.00 (m, 3H), 0.98-0.90 (m, 4H), 0.83-0.74 (m, 4H) |
| 97 | A | A | 1091.7 | ¹H NMR: (400 MHz, DMSO-d₆) δ: 9.00-8.99 (m, 1H), 8.41-8.20 |
| | | | | (m, 3H), 8.15 (s, 1H), 7.90-7.83 (m, 2H), 7.43 (d, J = 8.4 Hz, 2H), |
| | | | | 7.38-7.33 (m, 2H), 7.29-7.19 (m, 1H), 7.04-6.95 (m, 2H), 6.94- |
| | | | | 6.85 (m, 2H), 6.10 (s, 1H), 4.97-4.86 (m, 1H), 4.55-4.43 (m, 1H), |
| | | | | 4.35 (t, J = 7.6 Hz, 1H), 4.28 (br s, 1H), 3.86 (br d, J = 11.2 Hz, 1H), |
| | | | | 3.70 (br dd, J = 4.4, 10.4 Hz, 1H), 3.67-3.57 (m, 4H), 3.54 (s, 2H), |
| | | | | 2.76-2.66 (m, 3H), 2.63-2.52 (m, 3H), 2.45 (s, 4H), 2.29-2.15 (m, |
| | | | | 5H), 2.01 (br t, J = 9.1 Hz, 1H), 1.91-1.75 (m, 4H), 1.67 (br d, J = 10.4 |
| | | | | Hz, 4H), 1.54 (br d, J = 5.6 Hz, 3H), 1.48 (br d, J = 6.8 Hz, 6H), 1.37 (d, |
| | | | | J = 7.2 Hz, 4H), 1.09 (br d, J = 11.6 Hz, 3H), 0.97-0.91 (m, 3H), 0.83- |
| | | | | 0.76 (m, 3H) |
| 98 | A | A | 1091.7 | ¹H NMR: (400 MHz, DMSO-d₆) δ: 9.01-8.96 (m, 1H), 8.40 (d, |
| | | | | J = 7.6 Hz, 1H), 8.25-8.16 (m, 3H), 8.05-7.81 (m, 2H), 7.48-7.40 |
| | | | | (m, 3H), 7.38-7.35 (m, 2H), 7.32-7.22 (m, 1H), 7.00-6.87 (m, 3H), |
| | | | | 6.10 (s, 1H), 4.91 (m, 1H), 4.55-4.44 (m, 1H), 4.36 (m, 1H), 4.28 (s, |
| | | | | 1H), 3.78 (d, J = 9.6 Hz, 2H), 3.74-3.65 (m, 3H), 3.63-3.56 (m, 3H), |
| | | | | 2.76-2.67 (m, 3H), 2.64-2.53 (m, 1H), 2.45 (s, 4H), 2.22-2.08 (m, |
| | | | | 5H), 2.01 (m, 1H), 1.88 (d, J = 7.6 Hz, 8H), 1.54-1.40 (m, 10H), 1.37 |
| | | | | (d, J = 7.2 Hz, 4H), 1.18-0.99 (m, 3H), 0.97-0.92 (m, 3H), 0.83- |
| | | | | 0.77 (m, 3H) |
| 99 | A | A | 1091.7 | ¹H NMR: (400 MHz, DMSO-d₆) δ: 9.01-8.96 (m, 1H), 8.64-8.20 |
| | | | | (m, 3H), 8.15 (s, 1H), 8.07-7.81 (m, 2H), 7.46-7.42 (m, 3H), 7.39- |
| | | | | 7.34 (m, 2H), 7.30-7.23 (m, 1H), 7.01-6.96 (m, 2H), 6.92-6.87 (m, |
| | | | | 1H), 6.11 (s, 1H), 4.91 (m, 1H), 4.55-4.44 (m, 1H), 4.36 (m, 1H), |
| | | | | 4.28 (s, 1H), 3.79 (d, J = 10.8 Hz, 1H), 3.73-3.65 (m, 2H), 3.64-3.57 |
| | | | | (m, 3H), 3.42 (d, J = 10.4 Hz, 2H), 2.78-2.67 (m, 3H), 2.66-2.51 (m, |
| | | | | 3H), 2.45 (s, 4H), 2.36-2.11 (m, 5H), 2.02 (m, 1H), 1.90 (d, J = 7.6 |
| | | | | Hz, 2H), 1.83-1.67 (m, 5H), 1.59 (s, 2H), 1.55-1.46 (m, 5H), 1.45- |
| | | | | 1.34 (m, 7H), 1.12 (d, J = 11.6 Hz, 3H), 0.98-0.92 (m, 3H), 0.83-0.77 |
| | | | | (m, 3H) |
| 100 | A | A | 1113.7 | |
| 101 | A | A | 994.6 | |
| 102 | B | A | 994.6 | |
| 103 | A | A | 994.6 | |
| 104 | A | A | 994.6 | |
| 105 | A | A | 1081.7 | ¹H NMR (400 MHz, DMSO-d₆) δ: 13.44 (s, 1H), 9.04-8.96 (m, 1H), |
| | | | | 8.74 (s, 1H), 8.43-8.30 (m, 2H), 8.06 (dd, J = 1.6, 8.4 Hz, 1H), 7.84 |
| | | | | (s, 1H), 7.63 (s, 2H), 7.50-7.25 (m, 5H), 7.02-6.89 (m, 2H), 6.16- |
| | | | | 5.88 (m, 1H), 5.13-4.99 (m, 1H), 4.98-4.86 (m, 1H), 4.36 (t, J = 7.6 |
| | | | | Hz, 1H), 4.31-4.23 (m, 1H), 4.14-4.04 (m, 1H), 3.71 (br dd, J = 4.4, |
| | | | | 10.4 Hz, 1H), 3.66-3.52 (m, 3H), 3.41 (br d, J = 10.0 Hz, 2H), 3.27- |
| | | | | 3.16 (m, 2H), 2.98-2.84 (m, 2H), 2.83-2.60 (m, 5H), 2.46 (s, 3H), |
| | | | | 2.29-2.09 (m, 5H), 2.06-1.89 (m, 5H), 1.89-1.49 (m, 11H), 1.48- |
| | | | | 1.33 (m, 5H), 1.28-1.18 (m, 2H), 1.15-1.02 (m, 2H), 1.00-0.91 (m, |
| | | | | 3H), 0.86-0.72 (m, 3H) |
| 106 | A | A | 1080.8 | ¹H NMR: (400 MHz, DMSO-d₆) δ: 8.41 (d, J = 7.6 Hz, 1H), 8.20 (s, |
| | | | | 2H), 7.97-7.90 (m, 1H), 7.52-7.43 (m, 4H), 7.38 (d, J = 8.4 Hz, 2H), |
| | | | | 7.27-7.21 (m, 1H), 6.91-6.85 (m, 2H), 6.39-6.34 (m, 1H), 6.10 (s, |
| | | | | 1H), 5.88 (s, 2H), 4.93 (m, 1H), 4.36 (m, 1H), 4.28 (s, 1H), 3.84 (s, |
| | | | | 3H), 3.81-3.65 (m, 4H), 3.63-3.36 (m, 9H), 3.29-3.21 (m, 3H), |
| | | | | 2.91 (m, 2H), 2.85 (d, J = 10.8 Hz, 1H), 2.80-2.55 (m, 5H), 2.48-2.43 |
| | | | | (m, 1H), 2.42-2.29 (m, 4H), 2.26-2.17 (m, 3H), 2.02 (m, 1H), 1.88 |
| | | | | (s, 5H), 1.83-1.75 (m, 2H), 1.70 (d, J = 10.4 Hz, 3H), 1.64-1.55 (m, |
| | | | | 3H), 1.54-1.43 (m, 3H), 1.38 (d, J = 7.2 Hz, 3H), 1.17-1.03 (m, 2H), |
| | | | | 1.00-0.91 (m, 3H), 0.86-0.72 (m, 3H) |
| 107 | D | NA | 1037.6 | |
| 108 | A | A | 1127.8 | ¹H NMR: (400 MHz, DMSO-d₆) δ: 8.98 (s, 1H), 8.39 (d, J = 7.6 Hz, |
| | | | | 1H), 8.22 (s, 1H), 7.94 (d, J = 8.0 Hz, 1H), 7.50 (s, 1H), 7.47-7.41 (m, |
| | | | | 2H), 7.39-7.34 (m, 2H), 7.23 (m, J = 7.6 Hz, 1H), 6.91-6.85 (m, 2H), |
| | | | | 6.12 (s, 1H), 5.87 (s, 2H), 4.91 (m, 1H), 4.36 (m, 1H), 4.31-4.25 (m, |
| | | | | 1H), 3.73-3.62 (m, 7H), 3.28-3.22 (m, 3H), 3.21 (s, 2H), 2.91 (m, |
| | | | | 5H), 2.84-2.76 (m, 1H), 2.76-2.61 (m, 3H), 2.56 (d, J = 11.6 Hz, |
| | | | | 1H), 2.45 (s, 3H), 2.43 (d, J = 5.6 Hz, 1H), 2.39-2.26 (m, 4H), 2.22 |
| | | | | (m, 1H), 2.17-1.99 (m, 6H), 1.87 (s, 4H), 1.84-1.72 (m, 7H), 1.63- |
| | | | | 1.54 (m, 1H), 1.47-1.35 (m, 9H), 0.98-0.91 (m, 3H), 0.84-0.76 (m, 3H) |
| 109 | D | NA | 1102.8 | |
| 110 | A | A | 1102.8 | |
| 111 | D | NA | 1102.8 | |
| 112 | A | A | 1102.8 | |
| 113 | A | A | 1085.7 | |
| 114 | D | NA | 1029.7 | |
| 115 | D | NA | 1029.7 | |
| 116 | A | A | 1151.7 | ¹H NMR (400 MHz, CD₃OD) δ = 9.83 (s, 1H), 7.83 (d, J = 2.8 Hz, |
| | | | | 1H), 7.56-7.50 (m, 6H), 7.46-7.42 (m, 1H), 7.07-7.05 (m, 2H), 7.03- |
| | | | | 6.86 (m, 1H), 6.25 (s, 1H), 6.13 (s, 1H), 5.23 (s, 1H), 5.06-5.02(m, |
| | | | | 1H), 4.53(t, J = 2.8 Hz , 1H), 4.45(s, 2H),3.89-3.83(m, 6H),3.65- |
| | | | | 3.61(m, 3H), 3.49-3.47(m, 3H), 3.21-3.20(m, 5H), 2.75(s, 3H), 2.59(s, |
| | | | | 3H), 2.30-2.28(m, 1H), 2.22-2.20(m, 2H), 1.98-1.97(m, 3H), 1.96- |
| | | | | 1.95(m, 3H), 1.74-1.54(m, 2H), 1.52(d, J = 2.8 Hz, 1H), 1.51(d, J = |
| | | | | 5.6 Hz, 3H), 1.06-1.04(m, 2H), 1.03-1.01(m, 1H), 0.99(d, J = 5.6 Hz, |
| | | | | 3H), 0.88(d, J = 5.6 Hz, 3H) |
| 117 | A | A | 1151.7 | ¹H NMR (400 MHz, CD₃OD) δ = 9.87 (s, 1H), 7.85 (d, J = 2.8 Hz, |
| | | | | 1H), 7.58-7.54 (m, 6H), 7.49-7.45 (m, 1H), 7.11-7.08 (m, 2H), 7.08- |
| | | | | 6.91 (m, 1H), 6.26 (s, 1H), 6.16 (s, 1H), 5.24 (s, 1H), 5.03-5.00(m, |
| | | | | 1H), 4.53(t, J = 2.8 Hz, 1H), 4.43(s, 2H), 3.86-3.77(m, 6H), 3.74- |
| | | | | 3.71(m, 3H), 3.49-3.47(m, 3H), 3.21-3.20(m, 5H), 2.60(s, 3H), 2.30(s, |
| | | | | 3H), 2.29-2.28(m, 1H), 2.22-2.20(m, 2H), 1.98-1.97(m, 3H), 1.96- |
| | | | | 1.95(m, 3H), 1.74-1.54(m, 2H), 1.52(d, J = 2.8 Hz, 1H), 1.51(d, J = |
| | | | | 5.6 Hz, 3H), 1.06-1.04(m, 2H), 1.03-1.01(m, 1H), 1.02(d, J = 5.6 Hz, |
| | | | | 3H), 0.91(d, J = 5.6 Hz, 3H) |
| 118 | A | A | 1037.6 | |
| 119 | A | A | 1097.8 | ¹H NMR: (400 MHz, DMSO-d₆) δ: 14.24 (s, 1H), 8.99 (s, 1H), 8.40 |
| | | | | (d, J = 7.6 Hz, 1H), 7.96 (d, J = 7.2 Hz, 1H), 7.51 (s, 1H), 7.48-7.42 (m, |
| | | | | 2H), 7.39-7.35 (m, 2H), 7.25 (t, J = 7.6 Hz, 1H), 6.91-6.86 (m, 2H), |
| | | | | 6.10 (s, 1H), 5.88 (s, 2H), 5.11 (d, J = 3.6 Hz, 1H), 4.92 (t, J = 6.8 Hz, |
| | | | | 1H), 4.36 (t, J = 8.0 Hz, 1H), 4.28 (br s, 1H), 3.80-3.69 (m, 2H), 3.64- |
| | | | | 3.41 (m, 6H), 3.31-3.19 (m, 4H), 2.95-2.82 (m, 3H), 2.75-2.65 (m, |
| | | | | 3H), 2.47-2.40 (m, 5H), 2.32-1.97 (m, 8H), 1.88 (br s, 6H), 1.82- |
| | | | | 1.42 (m, 13H), 1.38 (d, J = 7.2 Hz, 3H), 1.08 (br d, J = 11.2 Hz, 2H), |
| | | | | 0.99-0.91 (m, 3H), 0.84-0.75 (m, 3H) |
| 120 | A | A | 1131.7 | ¹H NMR (400 MHz, DMSO-d₆) δ: 14.21 (br s, 1H), 8.98 (s, 1H), 8.39 |
| | | | | (d, J = 7.6 Hz, 1H), 7.93 (d, J = 7.2 Hz, 1H), 7.59-7.13 (m, 6H), 6.93- |
| | | | | 6.75 (m, 2H), 6.09 (s, 1H), 5.89 (s, 2H), 5.21-4.97 (m, 1H), 4.91 (t, |
| | | | | J = 7.2 Hz, 1H), 4.35 (t, J = 7.6 Hz, 1H), 4.30-4.21 (m, 1H), 3.70 |
| | | | | (dd, J = 4.4, 10.4 Hz, 1H), 3.63-3.49 (m, 3H), 3.37 (br s, 2H), 3.28- |
| | | | | 3.20 (m, 3H), 2.97-2.79 (m, 3H), 2.71 (br d, J = 8.4 Hz, 2H), 2.65- |
| | | | | 2.56 (m, 2H), 2.45 (s, 3H), 2.31-2.12 (m, 6H), 2.11-1.96 (m, 7H), |
| | | | | 1.95-1.84 (m, 7H), 1.82-1.74 (m, 1H), 1.67 (br d, J = 12.4 Hz, 3H), |
| | | | | 1.51 (br s, 4H), 1.44 (br d, J = 6.4 Hz, 2H), 1.37 (d, J = 7.2 Hz, 3H), |
| | | | | 1.23 (br s, 1H), 1.15-1.00 (m, 2H), 0.98-0.89 (m, 3H), 0.87-0.73 |
| | | | | (m, 3H) |
| 121 | A | A | 1091.7 | ¹H NMR (400 MHz, DMSO-d₆) δ: 8.99 (s, 1H), 8.59 (s, 1H), 8.40 (d, |
| | | | | J = 7.6 Hz, 1H), 8.23-8.18 (m, 3H), 8.01 (d, J = 8.0 Hz, 1H), 7.48- |
| | | | | 7.42 (m, 2H), 7.37-7.35(m, 2H), 7.25 (t, J = 8.0 Hz, 1H), 6.93-6.90 |
| | | | | (m, 2H), 6.52 (d, J = 4.0 Hz, 2H), 6.08 (s, 1H), 5.49-5.45 (m, 1H), |
| | | | | 4.92-4.89 (m, 1H), 4.41-4.33 (m, 2H), 4.29-4.27 (m, 2H), 3.87- |
| | | | | 3.83 (m, 2H), 3.72-3.62 (m, 2H), 3.58-3.51 (m, 2H), 2.71-2.66 (m, |
| | | | | 2H), 2.45 (m, 3H), 2.33-2.17 (m, 6H), 2.04-1.97 (m, 7H), 1.81- |
| | | | | 1.78 (m, 3H), 1.72 (dd, J = 2.8 Hz, 6.4 Hz), 1.65-1.61 (m, 2H), 1.45- |
| | | | | 1.40 (m, 3H), 1.38 (d, J = 6.8 Hz, 3H), 1.34-1.26 (m, 2H), 1.22-1.01 |
| | | | | (m, 8H), 0.99-0.96 (m, 1H), 0.95 (d, J = 6.8 Hz, 3H), 0.77 (d, J = 6.8 |
| | | | | Hz, 3H) |
| 122 | A | A | 1085.7 | ¹H NMR: (400 MHz, DMSO-d₆) δ: 14.33 (br s, 1H), 9.02-8.94 (m, |
| | | | | 1H), 8.82-8.36 (m, 1H), 7.93 (d, J = 7.2 Hz, 1H), 7.54-7.33 (m, |
| | | | | 5H), 7.27-7.18 (m, 1H), 6.92-6.84 (m, 2H), 6.18 (br s, 2H), 6.10 (s, |
| | | | | 1H), 5.20-5.00 (m, 1H), 4.92 (quin, J = 7.2 Hz, 1H), 4.37 (t, J = 7.6 |
| | | | | Hz, 1H), 4.29 (br s, 1H), 4.17 (s, 1H), 3.72 (br dd, J = 4.4, 10.4 Hz, |
| | | | | 1H), 3.66-3.51 (m, 3H), 3.49-3.37 (m, 2H), 3.24-3.13 (m, 2H), |
| | | | | 3.03 (br t, J = 10.4 Hz, 2H), 2.72 (br t, J = 11.2 Hz, 2H), 2.64-2.52 |
| | | | | (m, 4H), 2.46 (s, 3H), 2.41-1.95 (m, 14H), 1.95-1.74 (m, 5H), 1.74- |
| | | | | 1.55 (m, 5H), 1.54-1.41 (m, 6H), 1.38 (d, J = 7.2 Hz, 3H), 1.09 (br d, |
| | | | | J = 11.2 Hz, 2H), 0.99-0.91 (m, 3H), 0.86-0.75 (m, 3H) |
| 123 | A | A | 1136.7 | ¹H NMR: (400 MHz, METHANOL-d₄) δ = 9.54-9.44 (m, 1H), 7.86- |
| | | | | 7.78 (m, 1H), 7.60-7.41 (m, 7H), 7.10-7.00 (m, 2H), 6.90-6.84 (m, |
| | | | | 1H), 6.30-6.21 (m, 1H), 6.17-6.08 (m, 1H), 5.28-5.18 (m, 1H), |
| | | | | 5.09-5.00 (m, 1H), 4.82-4.66 (m, 4H), 4.54-4.49 (m, 1H), 4.47- |
| | | | | 4.37 (m, 2H), 3.93-3.80 (m, 3H), 3.70-3.54 (m, 5H), 3.38-3.33 (m, |
| | | | | 4H), 3.28-3.25 (m, 4H), 3.10-3.02 (m, 2H), 2.61-2.57 (m, 3H), |
| | | | | 2.57-2.55 (m, 3H), 2.42-2.35 (m, 1H), 2.32-2.26 (m, 2H), 2.25- |
| | | | | 2.15 (m, 3H), 1.99-1.92 (m, 1H), 1.92-1.83 (m, 2H), 1.62-1.54 (m, |
| | | | | 2H), 1.53 (d, J = 7.2 Hz, 3H), 1.38-1.20 (m, 1H), 1.09-1.01 (m, |
| | | | | 3H), 0.94-0.86 (m, 3H) |
| 124 | D | NA | 1136.7 | ¹H NMR: (400 MHz, METHANOL-d₄) δ = 9.54-9.44 (m, 1H), 7.86- |
| | | | | 7.78 (m, 1H), 7.60-7.41 (m, 7H), 7.10-7.00 (m, 2H), 6.90-6.84 (m, |
| | | | | 1H), 6.30-6.21 (m, 1H), 6.17-6.08 (m, 1H), 5.28-5.18 (m, 1H), |
| | | | | 5.09-5.00 (m, 1H), 4.82-4.66 (m, 4H), 4.54-4.49 (m, 1H), 4.47- |
| | | | | 4.37 (m, 2H), 3.93-3.80 (m, 3H), 3.70-3.54 (m, 5H), 3.38-3.33 (m, |
| | | | | 4H), 3.28-3.25 (m, 4H), 3.10-3.02 (m, 2H), 2.61-2.57 (m, 3H), |
| | | | | 2.57-2.55 (m, 3H), 2.42-2.35 (m, 1H), 2.32-2.26 (m, 2H), 2.25- |
| | | | | 2.15 (m, 3H), 1.99-1.92 (m, 1H), 1.92-1.83 (m, 2H), 1.62-1.54 (m, |
| | | | | 2H), 1.53 (d, J = 7.2 Hz, 3H), 1.38-1.20 (m, 1H), 1.09-1.01 (m, |
| | | | | 3H), 0.94-0.86 (m, 3H) |
| 125 | A | A | 1137.8 | |
| 126 | B | A | 1137.8 | |
| 127 | D | NA | 1112.7 | |
| 128 | A | A | 1081.6 | |
| 129 | B | A | 1038.6 | |
| 130 | C | A | 1081.7 | ¹H NMR (400 MHz, DMSO-d₆) δ: 13.20 (s, 1H), 8.98 (s, 1H), 8.40 |
| | | | | (d, J = 7.6 Hz, 1H), 8.17 (s, 1H), 8.03 (d, J = 1.2 Hz, 1H), 7.98-7.94 |
| | | | | (m, 1H), 7.46-7.41 (m, 2H), 7.38-7.34 (m, 3H), 7.30-7.25 (m, 1H), |
| | | | | 6.95-6.88 (m, 2H), 6.75 (s, 2H), 6.09 (s, 1H), 5.11 (br, 1H), 4.95- |
| | | | | 4.85 (m, 1H), 4.36 (t, J = 7.6 Hz, 1H), 4.28 (s, 1H), 4.10-4.02 (m, |
| | | | | 1H), 3.75-3.68 (m, 1H), 3.63-3.52 (m, 4H), 3.45-3.37 (m, 2H), |
| | | | | 2.86-2.80 (m, 2H), 2.75-2.60 (m, 7H), 2.45 (s, 3H), 2.27-2.20 (m, |
| | | | | 1H), 2.12-2.06 (m, 4H), 1.96-1.87 (m, 5H), 1.75-1.67 (m, 7H), |
| | | | | 1.65-1.56 (m, 5H), 1.47-1.40 (m, 1H), 1.37 (d, J = 6.4 Hz, 3H), 1.25- |
| | | | | 1.15 (m, 2H), 1.10-1.04 (m, 1H), 0.94 (d, J = 6.4 Hz, 3H), 0.78 (d, |
| | | | | J = 6.4 Hz, 3H) |
| 131 | D | NA | 1081.6 | |
| 132 | A | A | 1110.8 | ¹H NMR: (400 MHz, DMSO-d₆) δ: 9.09-8.93 (m, 1H), 8.39 (d, J = |
| | | | | 7.6 Hz, 1H), 8.15 (s, 1H), 8.08-7.84 (m, 1H), 7.54-7.33 (m, 5H), |
| | | | | 7.29-7.22 (m, 1H), 7.07-6.83 (m, 2H), 6.13 (s, 1H), 6.03-5.84 (m, |
| | | | | 2H), 5.12 (br d, J = 6.0 Hz, 1H), 4.97-4.84 (m, 1H), 4.36 (t, J = 7.6 |
| | | | | Hz, 1H), 4.29 (br s, 1H), 3.72 (br dd, J = 4.0, 10.4 Hz, 2H), 3.66-3.52 |
| | | | | (m, 5H), 3.46-3.41 (m, 2H), 3.32 (br s, 4H), 3.12-3.02 (m, 2H), 2.92 |
| | | | | (br t, J = 9.6 Hz, 4H), 2.80-2.63 (m, 8H), 2.58 (br s, 3H), 2.46 (s, |
| | | | | 3H), 2.42-2.37 (m, 1H), 2.26-2.15 (m, 2H), 2.13-1.87 (m, 8H), |
| | | | | 1.84-1.54 (m, 10H), 1.47-1.33 (m, 3H), 1.22-1.10 (m, 2H), 1.01- |
| | | | | 0.91 (m, 3H), 0.84-0.73 (m, 3H) |
| 133 | A | A | 1110.8 | ¹H NMR: (400 MHz, DMSO-d₆) δ: 9.09-8.94 (m, 1H), 8.38 (d, J = |
| | | | | 7.6 Hz, 1H), 8.14 (s, 2H), 8.08-7.82 (m, 1H), 7.58-7.33 (m, 5H), |
| | | | | 7.30-7.20 (m, 1H), 7.08-6.84 (m, 2H), 6.37-6.09 (m, 1H), 6.04- |
| | | | | 5.83 (m, 2H), 5.20-4.77 (m, 2H), 4.42-4.23 (m, 2H), 3.72 (br dd, J = |
| | | | | 4.4, 10.0 Hz, 1H), 3.67-3.52 (m, 5H), 3.42 (br d, J = 9.6 Hz, 4H), |
| | | | | 3.29 (br s, 3H), 3.10-3.00 (m, 2H), 2.98-2.82 (m, 4H), 2.80-2.61 |
| | | | | (m, 8H), 2.55 (br s, 2H), 2.46 (s, 3H), 2.42-2.36 (m, 1H), 2.31-2.11 |
| | | | | (m, 3H), 2.09-1.86 (m, 8H), 1.83-1.52 (m, 10H), 1.47-1.30 (m, |
| | | | | 3H), 1.21-1.09 (m, 2H), 1.01-0.90 (m, 3H), 0.85-0.74 (m, 3H) |
| 134 | D | NA | 1112.6 | |
| 135 | D | NA | 1052.6 | |
| 136 | A | A | 1052.6 | |
| 137 | D | NA | 1063.7 | |
| 138 | A | A | 1063.7 | |
| 139 | D | NA | 1064.7 | |
| 140 | A | A | 1064.1 | ¹H NMR (400 MHz, CD3OD): δ 7.78-7.75 (m, 2H), 7.74-7.68 (m, |
| | | | | 2H), 7.50-7.46 (m, 3H), 7.25-7.20 (m, 1H), 6.91-6.89 (m, 2H), 6.57- |
| | | | | 6.54 (m, 1H), 6.15-6.13 (m, 2H), 5.16-5.13 (m, 1H), 5.11-5.00 |
| | | | | (m, 2H), 4.87-4.41 (m, 5H), 3.65-3.62 (m, 1H), 3.59-3.55 (m, 5H), |
| | | | | 3.37-3.35 (m, 3H), 3.27-3.25 (m, 5H), 3.21-3.01 (m, 5H), 2.49- |
| | | | | 2.42 (m, 5H), 2.25-2.14 (m, 5H), 1.94-1.84 (m, 3H), 1.57-1.48 (m, |
| | | | | 5H), 1.05-1.02 (m, 3H), 0.89-0.87 (m, 3H) |
| 141 | A | A | 1069.7 | |
| 142 | A | A | 1097.8 | |
| 143 | A | A | 1097.8 | |
| 144 | B | A | 1055.6 | |
| 145 | B | A | 1125.7 | ¹H NMR (400 MHz, METHANOL-d₄) δ: = 8.89-8.84 (m, 1H), 7.84- |
| | | | | 7.73 (m, 2H), 7.60-7.53 (m, 1H), 7.49-7.34 (m, 4H), 7.31-7.21 (m, |
| | | | | 1H), 6.98-6.87 (m, 2H), 6.64-6.58 (m, 1H), 6.22-6.16 (m, 1H), |
| | | | | 6.16-6.04 (m, 1H), 4.46-4.41 (m, 1H), 3.94-3.86 (m, 1H), 3.83- |
| | | | | 3.75 (m, 2H), 3.74-3.62 (m, 2H), 3.59 (br s, 4H), 3.51-3.42 (m, 2H), |
| | | | | 3.29-3.25 (m, 4H), 3.23-3.12 (m, 4H), 2.93-2.82 (m, 2H), 2.53- |
| | | | | 2.44 (m, 3H), 2.41-2.32 (m, 1H), 2.23-2.17 (m, 1H), 2.07-2.02 (m, |
| | | | | 2H), 2.00 (br d, J = 4.4 Hz, 1H), 1.98-1.92 (m, 2H), 1.88-1.79 (m, |
| | | | | 2H), 1.73 (br d, J = 8.4 Hz, 2H), 1.64-1.56 (m, 1H), 1.55-1.46 (m, |
| | | | | 4H), 1.35-1.27 (m, 6H), 1.08-0.94 (m, 3H), 0.92-0.87 (m, 3H) |
| 146 | C | A | 1015.6 | |
| 147 | B | A | 1015.6 | |
| 148 | A | A | 1059.6 | |
| 149 | D | NA | 1042.6 | |
| 150 | D | NA | 1009.6 | |
| 151 | B | A | 1009.6 | |
| 152 | D | NA | 1079.7 | |
| 153 | A | A | 1079.7 | |
| 154 | D | NA | 983.6 | |
| 155 | C | A | 983.6 | |
| 156 | A | A | 997.6 | |
| 157 | A | A | 997.8 | ¹H NMR (400 MHz, CD₃OD): δ 8.44-8.34 (m, 1H), 8.39 (s, 1H), |
| | | | | 7.81-7.80 (m, 1H), 7.76-7.74 (m, 1H), 7.68-7.64 (m, 2H), 7.53- |
| | | | | 7.41 (m, 3H), 7.25-7.23 (m, 1H), 6.92-6.87 (m, 2H), 6.59-6.57 (m, |
| | | | | 1H), 6.27 (s, 1H), 6.12-6.03 (m, 1H), 4.66-4.62 (m, 5H), 4.57-4.36 |
| | | | | (m, 3H), 3.84-3.82 (m, 1H), 3.75-3.56 (m, 4H), 3.52-3.40 (m, 1H), |
| | | | | 3.36-3.34 (m, 3H), 3.13-3.11 (m, 2H), 2.98-2.79 (m, 3H), 2.76- |
| | | | | 2.61 (m, 1H), 2.58-2.42 (m, 3H), 2.38-2.34 (m, 1H), 2.30-2.09 (m, |
| | | | | 6H), 2.06-1.89 (m, 2H), 1.85-1.82 (m, 2H), 1.60-1.44 (m, 3H), |
| | | | | 1.31-1.24 (m, 2H), 1.05-1.01 (m, 3H), 0.95-0.83 (m, 3H). |

*DC50 (nM):
A < 2.5
2.5 ≤ B < 10
10 ≤ C < 30
D ≥ 30
NA not calculated/no curve fit
**Dmax (% degraded):
A > 75
50 < B ≤ 75
C ≤ 50
NA not calculated/no curve fit

The contents of all references, patents, pending patent applications and published patents, cited throughout this application are hereby expressly incorporated by reference.

Those skilled in the art will recognize, or be able to ascertain using no more than routine experimentation, many equivalents to the specific embodiments of the disclosure described herein. Such equivalents are intended to be encompassed by the following claims. It is understood that the detailed examples and embodiments described herein are given by way of example for illustrative purposes only, and are in no way considered to be limiting to the disclosure. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present disclosure will be apparent from the appended claims. Moreover, those skilled in the art will recognize, or be able to ascertain using no more than routine experimentation, many equivalents to the specific embodiments of the disclosure described herein. Such equivalents are intended to be encompassed by the following claims.

What is claimed is:

1. A bifunctional compound having the chemical structure:
PTM-L-ULM,
or a pharmaceutically acceptable salt, enantiomer, stereoisomer, solvate, polymorph or prodrug thereof,
wherein:
(a) the L is a chemical linking moiety connecting the ULM and the PTM, and has a chemical structural unit represented by the formula
-(A^{L})_{q}-,
wherein:
(A^{L})_{q} is a group which is connected to at least one of ULM, PTM, or both;
q is an integer greater than or equal to 1;
each A^{L} is independently selected from the group consisting of CR^{L1}R^{L2}, O, SO₂, NR^{L3}, CONR^{L3}, CO, CR^{L1}═CR^{L2}, C≡C, C₃₋₁₁cycloalkyl optionally substituted with 1-6 R^{L1} and/or R^{L2} groups, C₃₋₁₁heteocyclyl optionally substituted with 1-6 R^{L1} and/or R^{L2} groups, aryl optionally substituted with 1-6 R^{L1} and/or R^{L2} groups, and heteroaryl optionally substituted with 1-6 R^{L1} and/or R^{L2} groups, where R^{L1} or R^{L2}, each independently are optionally linked to other groups to form cycloalkyl and/or heterocyclyl moiety, optionally substituted with 1-4 R^{L5} groups; and
R^{L1}, R^{L2}, R^{L3}, R^{L} and R^{L5} are, each independently, halogen, C₁₋₈alkyl, OC₁₋₈alkyl, NHC₁₋₈alkyl, N(C₁₋₈alkyl)₂, C₃₋₁₁cycloalkyl, aryl, heteroaryl, C₃₋₁₁heterocyclyl, OC₃₋₈cycloalkyl, NHC₃₋₈cycloalkyl, N(C₃₋₈cycloalkyl)(C₁₋₈alkyl), OH, NH₂, CC—C₁₋₈alkyl, CCH, CH═CH(C₁₋₈alkyl), C(C₁₋₈alkyl)=CH(C₁₋₈alkyl), C(C₁₋₈alkyl)=C(C₁₋₈alkyl)₂, COC₁₋₈alkyl, CO₂H, CN, CF₃, CHF₂, CH₂F, NO₂CONHC₁₋₈alkyl, or CON(C₁₋₈alkyl)₂; and
(b) the ULM is an E3 ubiquitin ligase binding moiety that binds a Von Hippel-Lindau E3 ubiquitin ligase and has a chemical structure represented by: 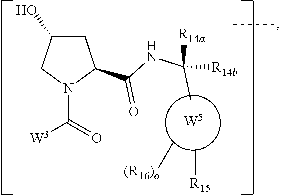
wherein:
W³ is selected from the group of an optionally substituted aryl, optionally substituted heteroaryl, or 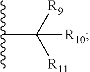
R₉ and R₁₀ are independently hydrogen, optionally substituted alkyl, optionally substituted cycloalkyl, optionally substituted hydroxyalkyl, optionally substituted heteroaryl, or haloalkyl, or R₉, R₁₀, and the carbon atom to which they are attached form an optionally substituted cycloalkyl;
R₁₁ is selected from the group of an optionally substituted heterocyclyl, optionally substituted alkoxy, optionally substituted heteroaryl, optionally substituted aryl, 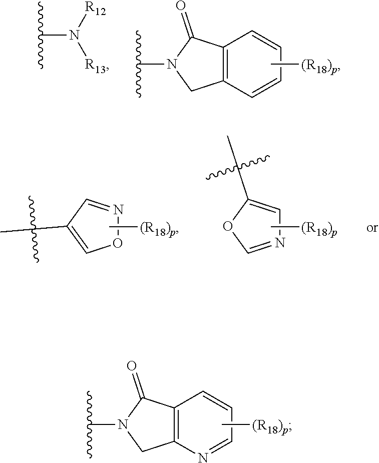
R₁₂ is selected from the group of H or optionally substituted alkyl;
R₁₃ is selected from the group of H, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (heterocyclyl)carbonyl, or optionally substituted aralkyl;
R₁₄ₐ, R_{14b}, are each independently selected from the group of H, amine, haloalkyl, optionally substituted alkyl, optionally substituted alkoxy, optionally substituted hydroxyl alkyl, optionally substituted alkylamine, optionally substituted amide, optionally substituted alkyl-amide, optionally substituted alkyl-cyano, optionally substituted alkyl-phosphate, optionally substituted heteroalkyl, optionally substituted alkyl-heterocycloalkyl, optionally substituted alkoxy-heterocycloalkyl, COR₂₆, alkyl-COR₂₆, CONR₂₇ₐR_{27b}, NHCOR₂₆, or NHCH₃COR₂₆, and the other of R₁₄ₐ and R_{14b} is H; or R₁₄ₐ, R_{14b}, together with the carbon atom to which they are attached, form an optionally substituted 3 to 5 membered cycloalkyl, heterocycloalkyl, spirocycloalkyl or spiroheterocyclyl, wherein the spiroheterocyclyl is not epoxide or aziridine;
W⁵ is optionally substituted phenyl, optionally substituted napthyl, or an optionally substituted 5-10 membered heteroaryl;
R₁₅ is selected from the group of H, halogen, CN, C≡CH, OH, NO₂, NR₂₇ₐR_{27b}, OR₂₇ₐ, CONR₂₇ₐR_{27b}, NR₂₇ₐCOR_{27b}, SO₂NR₂₇ₐR_{27b}, NR₂₇ₐSO₂R_{27b}, optionally substituted alkyl, optionally substituted haloalkyl, optionally substituted haloalkoxy; optionally substituted aryl; optionally substituted heteroaryl; optionally substituted cycloalkyl; or optionally substituted heterocyclyl;
each R₁₆ is independently selected from the group of halogen, CN, optionally substituted alkyl, optionally substituted alkylamine, optionally substituted haloalkyl, hydroxy, or optionally substituted haloalkoxy;
o is 0, 1, 2, 3, or 4;
R₁₈ is independently selected from the group of H, halogen, optionally substituted alkoxy, cyano, optionally substituted alkyl, haloalkyl, haloalkoxy or a linker;
each R₂₆ is independently selected from H, OH, optionally substituted alkyl or NR₂₇ₐR_{27b};
each R₂₇ₐ and R_{27b} is independently H, optionally substituted alkyl, optionally substituted cycloalkyl, or R₂₇ₐ and R_{27b} together with the nitrogen atom to which they are attached form a 4-6 membered heterocyclyl;
p is 0, 1, 2, 3, or 4, and
the [symbol] of the ULM indicates the site of attachment of a chemical linking moiety the PTM to the ULM; and
(c) the PTM is a small molecule SMARCA2 protein targeting moiety having a chemical structure selected from: 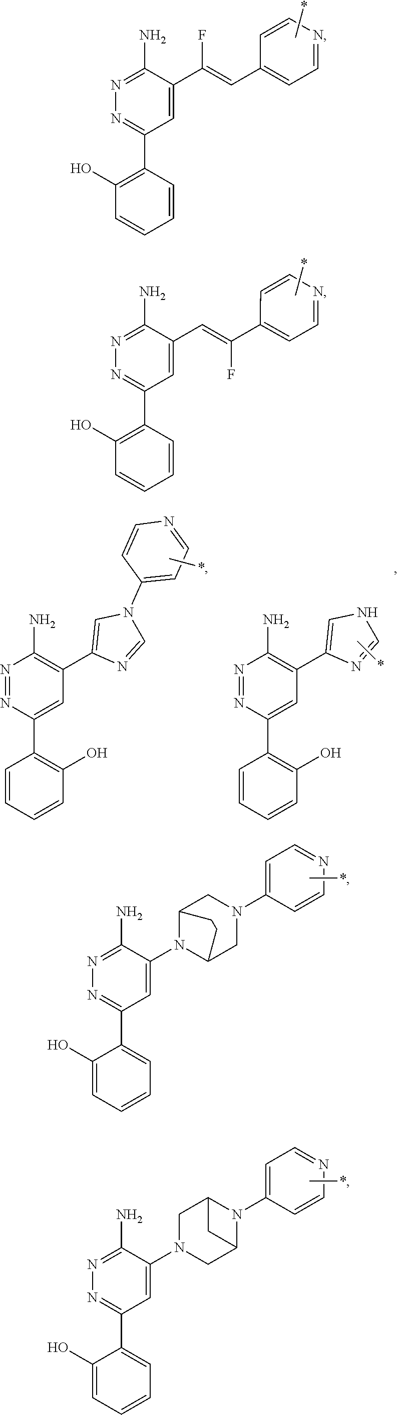 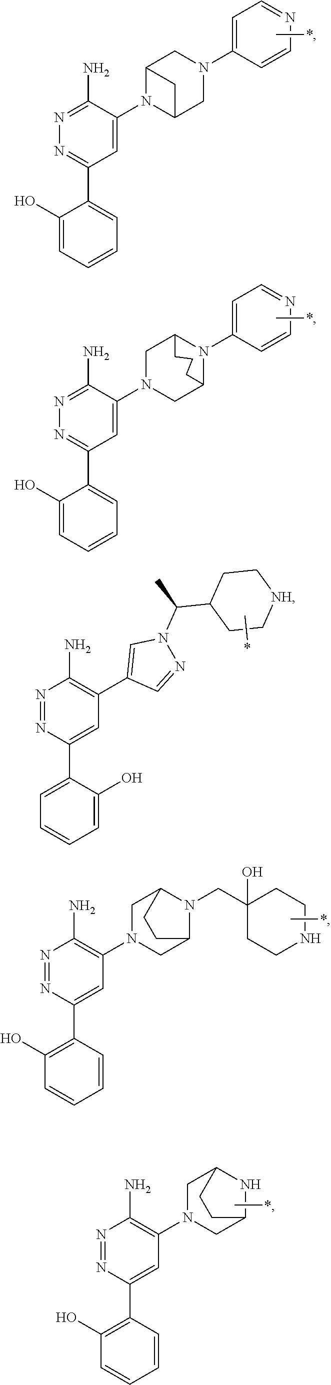 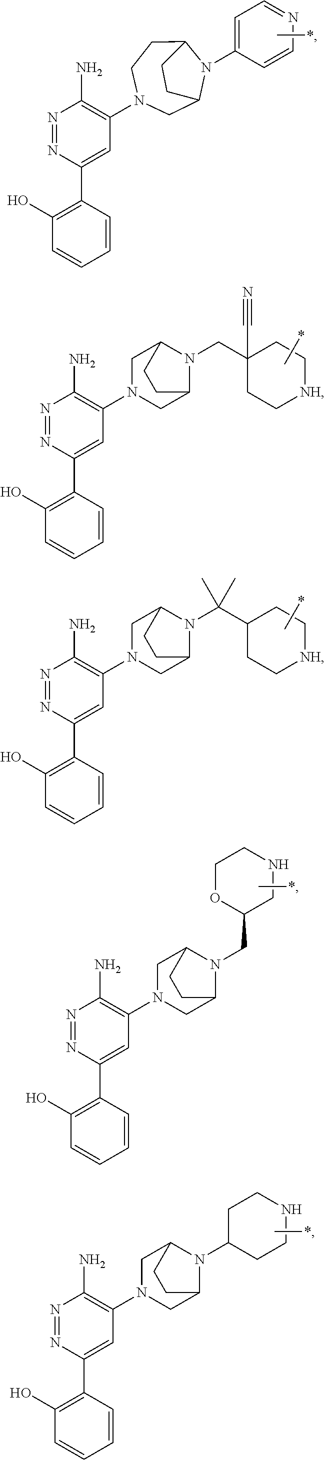 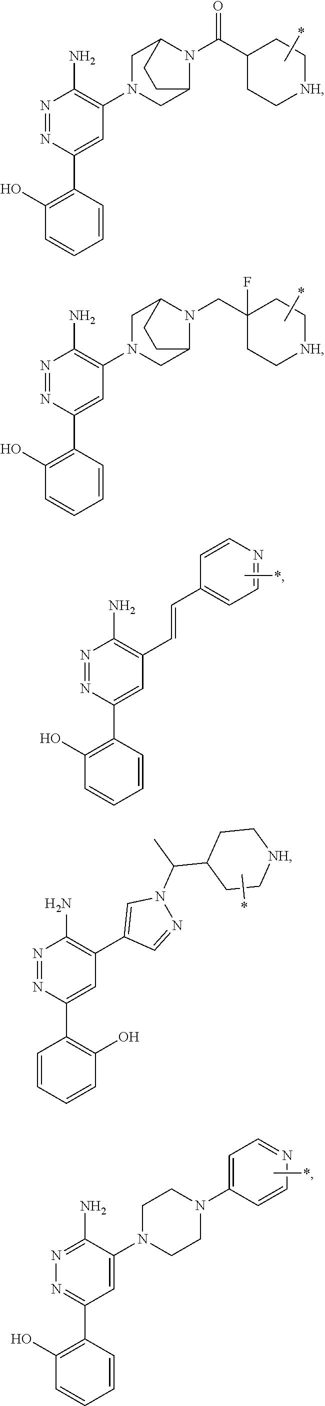 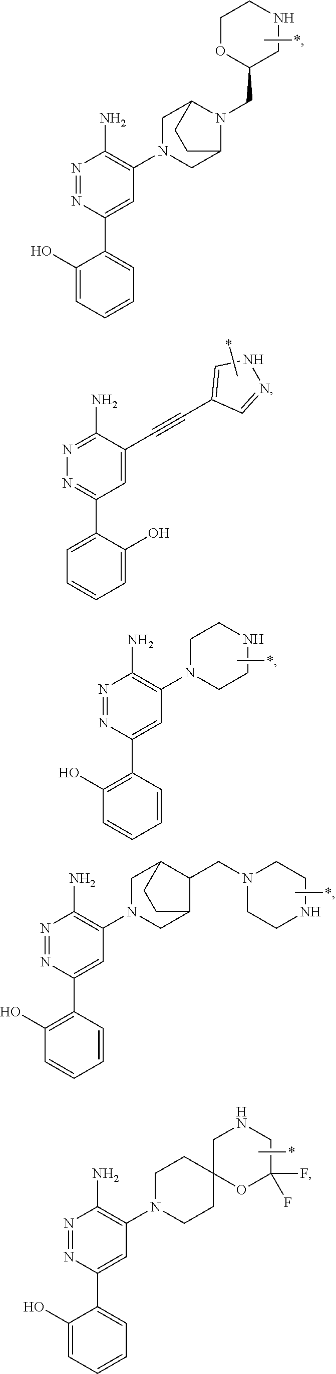 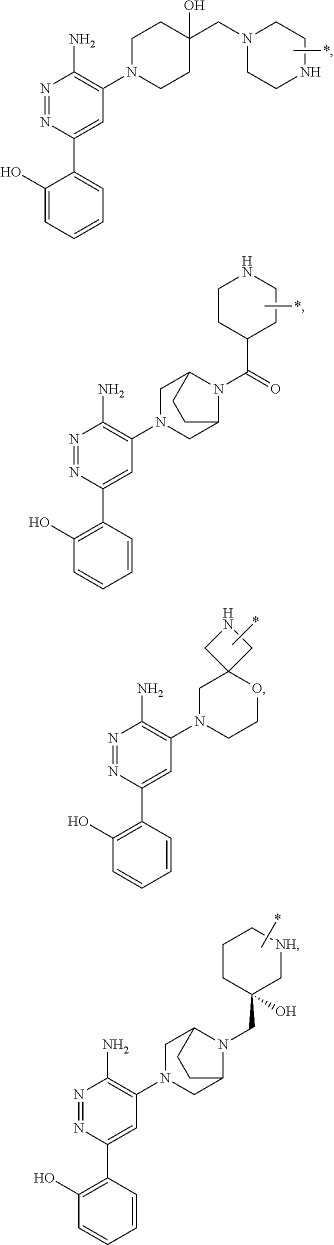 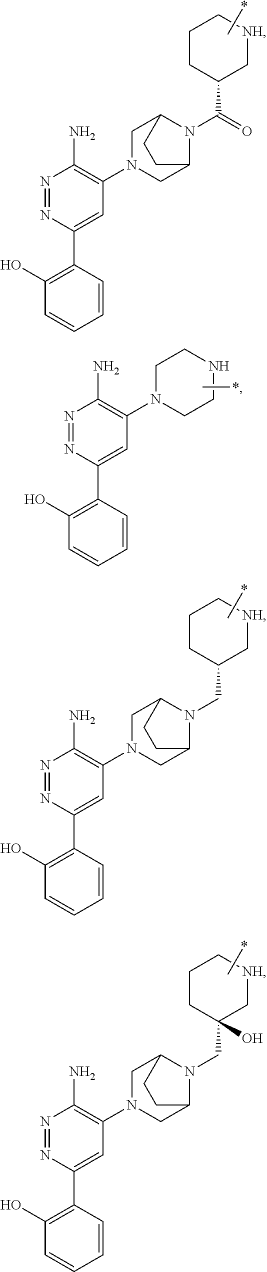 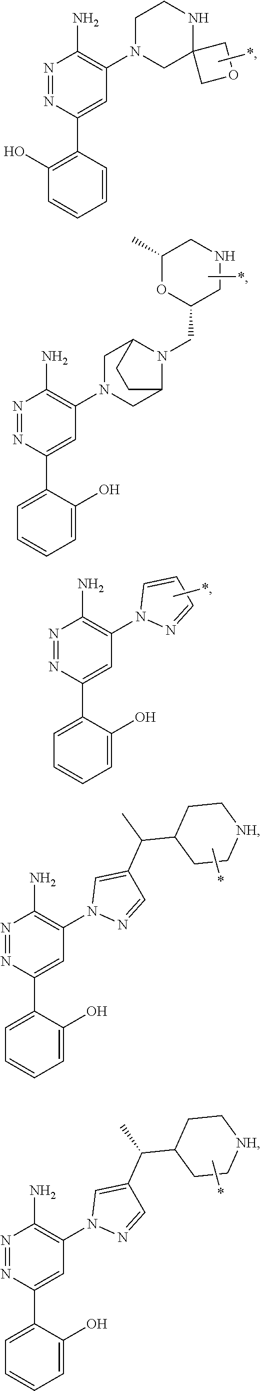 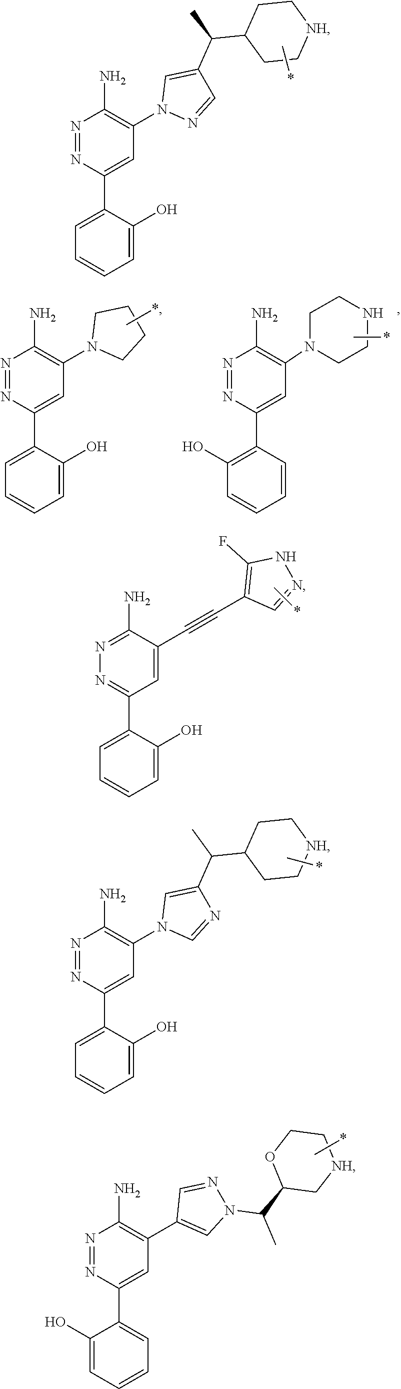 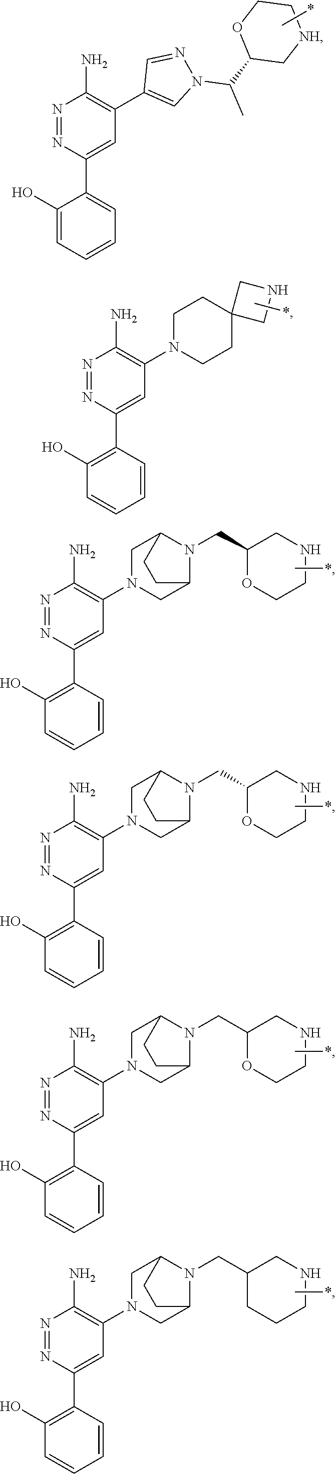 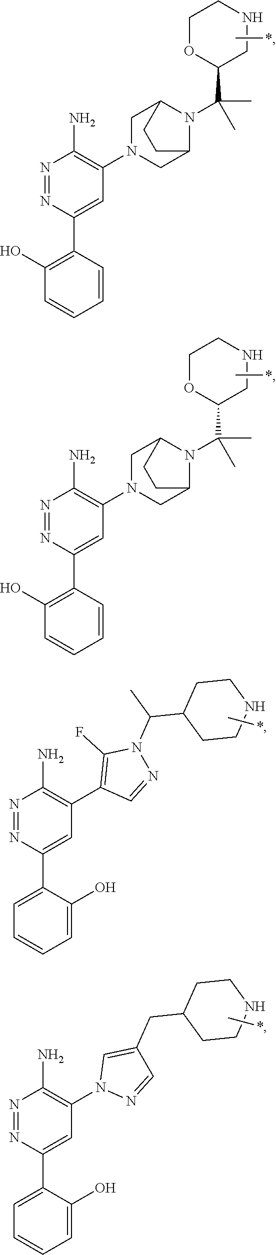 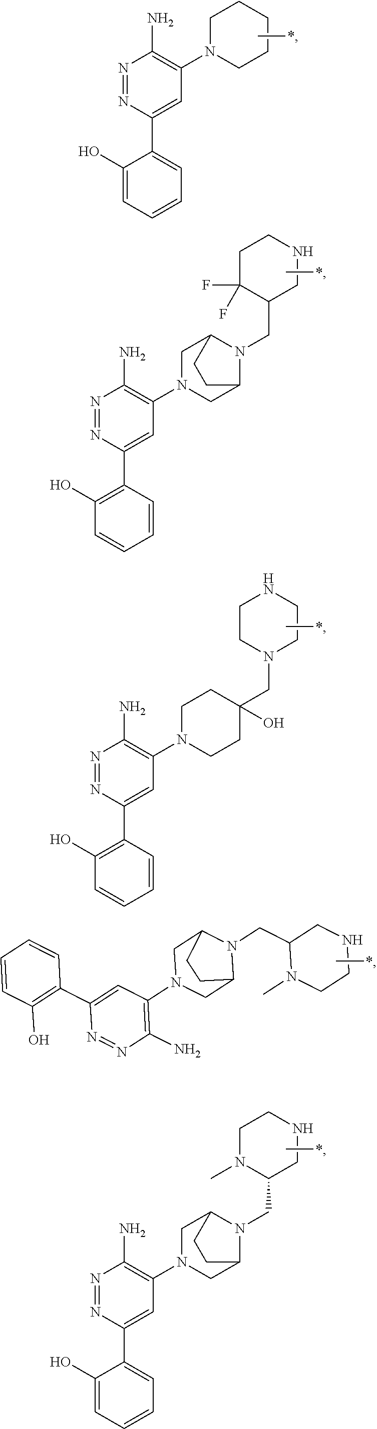 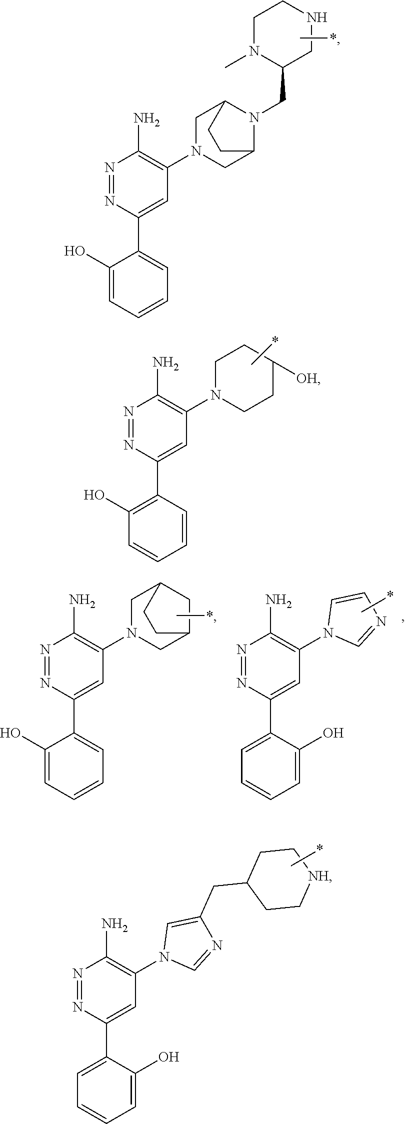 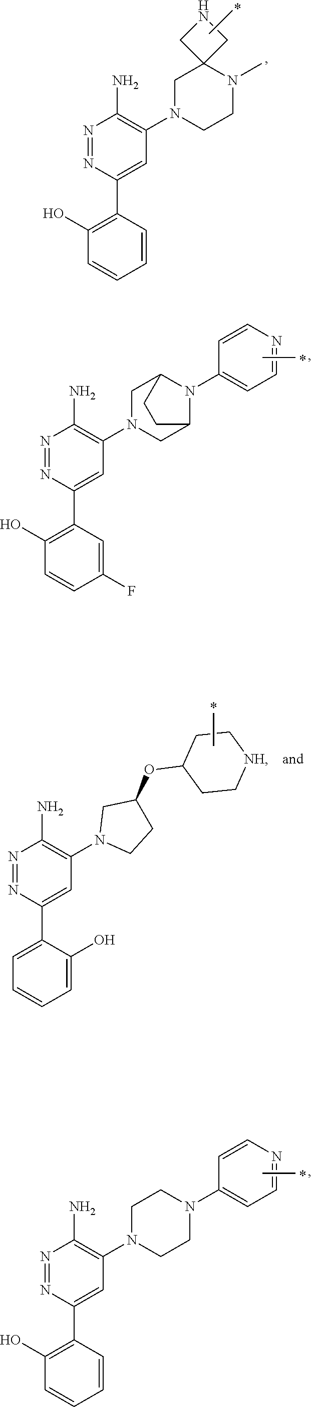
wherein [symbol] is the attachment point to the chemical linking moiety (e.g., the chemical linking moiety is attached to a carbon of the indicated ring or a non-aryl nitrogen of the indicated ring).

2. The compound according to claim 1, wherein the PTM is selected from the group consisting of: 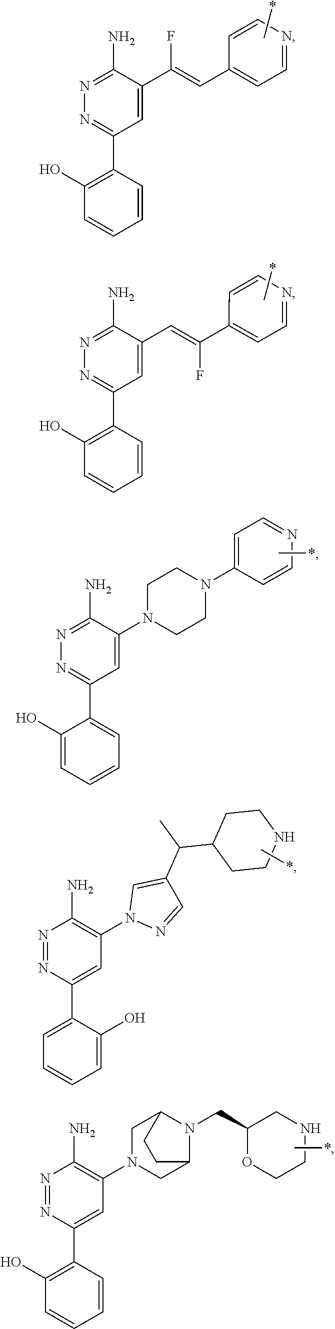 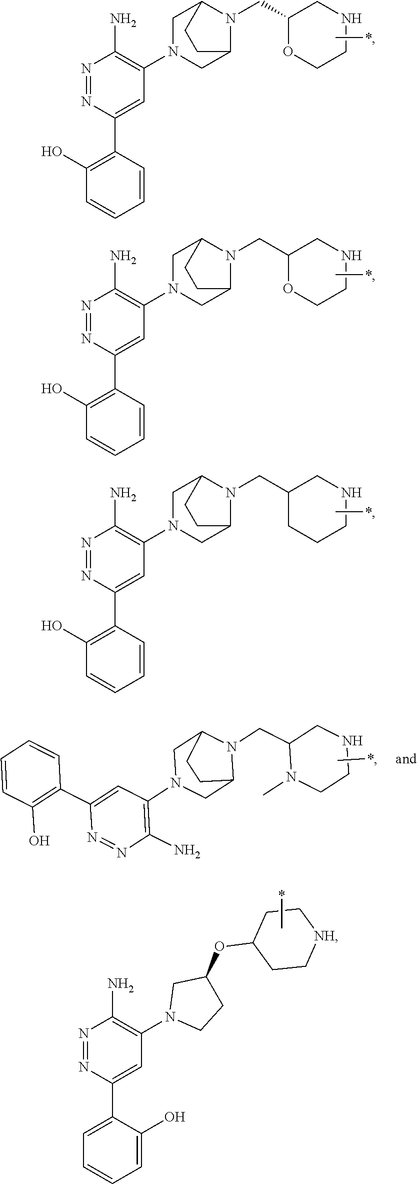
wherein [symbol] is the attachment point to the chemical linking moiety (e.g., the chemical linking moiety is attached to a carbon of the indicated ring or a non-aryl nitrogen of the indicated ring).

3. The compound according to claim 1 or 2, wherein the compound has a structure selected from: 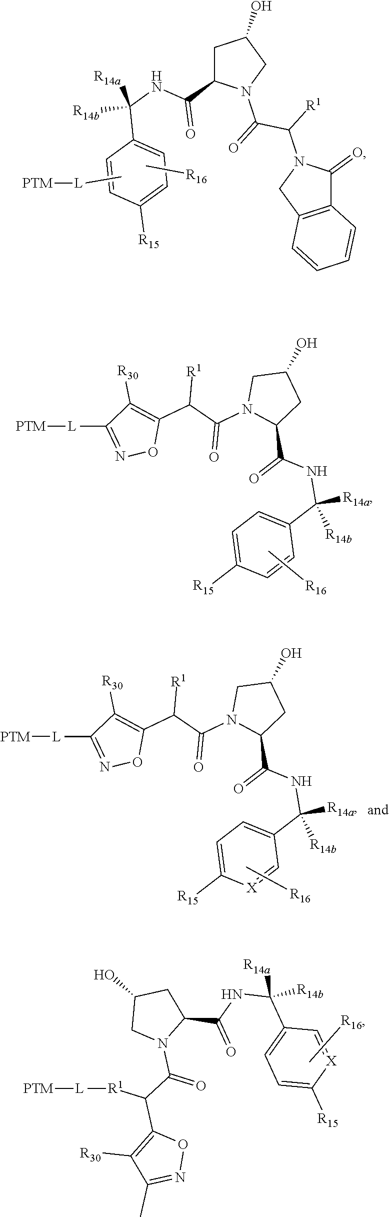
or a pharmaceutically acceptable salt thereof,
wherein:
X is CH or N;
R³⁰ is H, F or Cl; and
R₁ is a C₁₋₆ alkyl.

4. The compound according to claim 3, wherein one of R₁₄ₐ and R_{14b} is a H, methyl, C1 fluoroalkyl, CHF₂, CF₃, and the other is a H.

5. The compound according to claim 3 or 4, wherein R₁₅ is selected from cyano, halogen (e.g., fluoro or chloro),

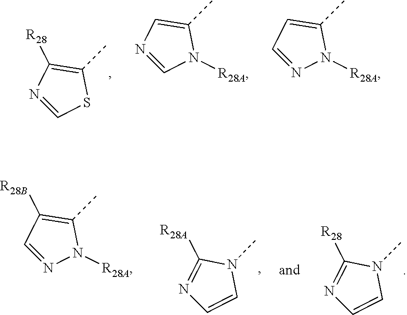

6. The compound of any one of claim 1-5, wherein:
each R¹⁶ is individually selected from H, C₁₋₄alkyl, fluoro, chloro, NH₂, CN, and C₁₋₄alkoxy;
R_{28A} is selected from H or methyl;
R_{28B} is selected from H, methyl, fluoro, and chloro; and
R₂₈ is selected from H, methyl, CH₂N(Me)₂, CH₂OH, CH₂O(C₁₋₄alkyl), CH₂NHC(O)C₁₋₄alkyl, NH₂,

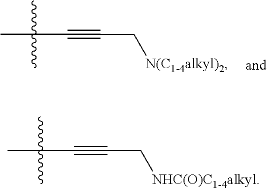

7. The compound according to claim 1 or 2, wherein the ULM has a chemical structure selected from: 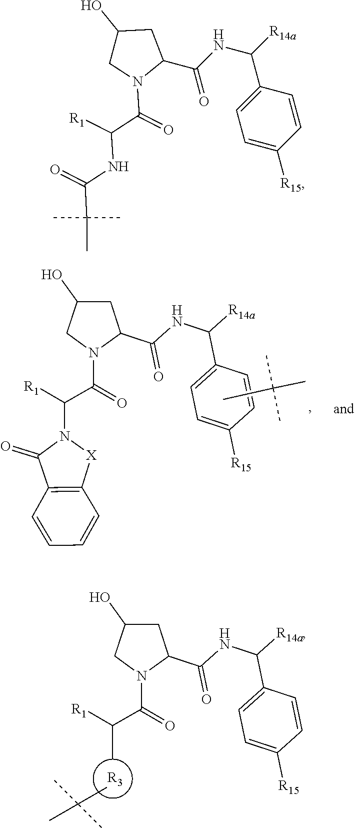
or a pharmaceutically acceptable salt thereof, wherein:
R₁ is H, optionally substituted alkyl, optionally substituted cycloalkyl, optionally substituted hydroxyalkyl, optionally substituted heteroaryl, or haloalkyl;
R₁₄ₐ is H, haloalkyl, optionally substituted alkyl, methyl, fluoromethyl, hydroxymethyl, ethyl, isopropyl, or cyclopropyl;
R₁₅ is selected from the group consisting of H, halogen, CN, C≡CH, OH, NO₂, optionally substituted heteroaryl, optionally substituted aryl, optionally substituted alkyl, optionally substituted haloalkyl, optionally substituted alkoxy, optionally substituted haloalkoxy, optionally substituted cycloalkyl, or optionally substituted heterocyclyl;
X is C, CH₂, or C═O;
R₃ is absent or an optionally substituted 5 or 6 membered heteroaryl; and
the 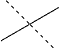
indicates the site of attachment of the chemical linking moiety coupling the PTM to the ULM.

8. The compound of claim 7, wherein the ULM is of the formula: 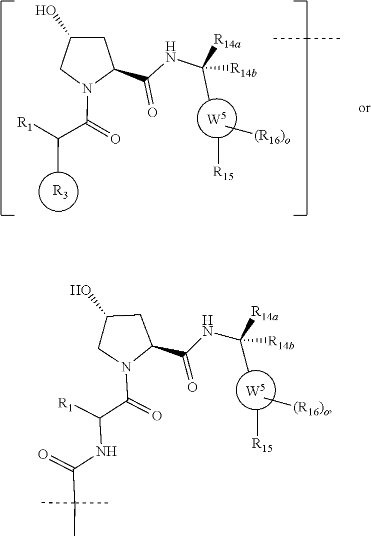
or a pharmaceutically acceptable salt thereof, wherein:
R₁ is H, optionally substituted alkyl or optionally substituted cycloalkyl;
R₃ is an optionally substituted 5-6 membered heteroaryl;
W⁵ is optionally substituted phenyl, optionally substituted napthyl or optionally substituted pyridinyl;
one of R₁₄ₐ and R_{14b} is H, optionally substituted alkyl, haloalkyl, optionally substituted alkoxy, optionally substituted hydroxyl alkyl, optionally substituted alkylamine, optionally substituted amide, optionally substituted alkyl-amide, optionally substituted alkyl-cyano, or optionally substituted heteroalkyl; and the other of R₁₄ₐ and R_{14b} is H;
R₁₅ is CN, C≡CH, fluoroalkyl, 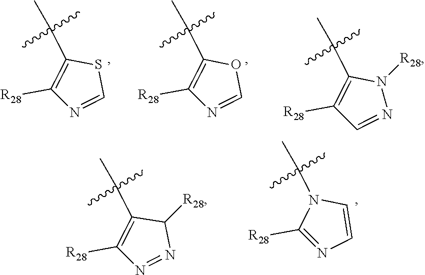
or optionally substituted 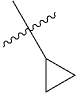 [SMILES: *C1CC1]
(e.g., 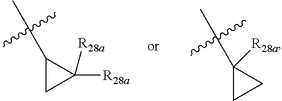
wherein R₂₈ₐ is halogen, optionally substituted alkyl, or fluoroalkyl);
each R₁₆ is independently selected from halo, CN, optionally substituted alkyl, optionally substituted haloalkyl, hydroxy, or haloalkoxy;
each R₂₇ₐ and R_{27b} is independently H, optionally substituted alkyl, optionally substituted 3-5 membered cycloalkyl, or R₂₇ₐ and R_{27b} together with the nitrogen atom to which they are attached form a 4-6 membered heterocyclyl;
R₂₈ is H, optionally substituted alkyl, optionally substituted alkoxy, optionally substituted heteroalkyl, optionally substituted alkylamine, optionally substituted hydroxyalkyl, amine, optionally substituted alkynyl, or optionally substituted cycloalkyl;
o is 0, 1 or 2; and
the 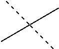
and the [symbol] of the ULM indicates the site of attachment of the chemical linking moiety coupling the PTM to the ULM.

9. The compound of claim 1, 2, or 8, wherein the ULM has a chemical structure selected from: 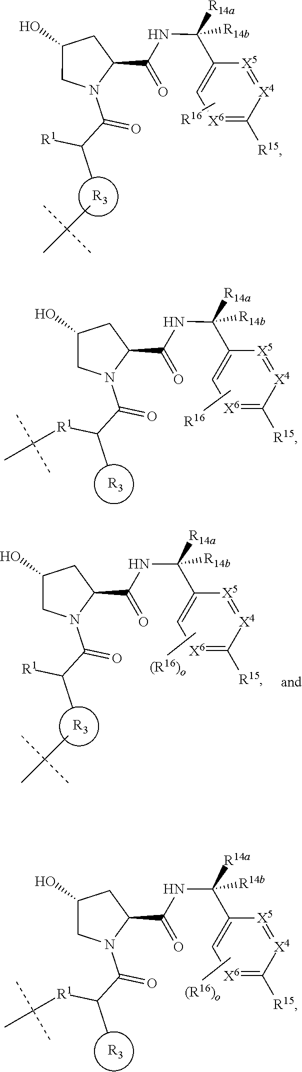
wherein:
is 0, 1, or 2;
each of X⁴, X⁵, and X⁶ is selected from CH and N, wherein no more than 2 are N;
R¹ is C₁₋₆ alkyl;
one of R^{14a} and R^{14b} is H, optionally substituted alkyl, optionally substituted haloalkyl, optionally substituted alkoxy, optionally substituted hydroxyl alkyl, optionally substituted alkylamine, optionally substituted amide, optionally substituted alkyl-amide, optionally substituted alkyl-cyano, or optionally substituted heteraolkyl; and
the other of R^{14a} and R^{14b} is H;
each R₂₇ₐ and R_{27b} is independently H or C₁₋₆ alkyl or a 3-5 membered cycloalkyl;
R¹⁵ is, 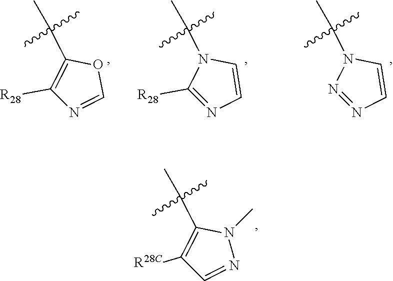
R²⁸ is H, methyl, CH₂N(Me)₂, CH₂OH, CH₂O(C₁₋₄alkyl), CH₂NHC(O)C₁₋₄alkyl, NH₂, 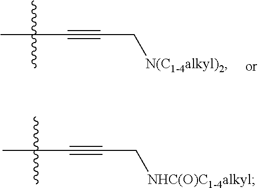
R^{28C} is H, methyl, fluoro, or chloro; and
R¹⁶ is H, C₁₋₄alkyl, fluoro, chloro, CN, or C₁₋₄alkoxy.

10. The compound of claim 9, wherein at least one of:
one R^{14a} and R^{14b} are selected from: H, C₁₋₄ alkyl, C₁₋₄ cycloalkyl, C₁₋₄ haloalkyl, C₁₋₄ hydroxyalkyl, C₁₋₄ alkyloxyalkyl, C₁₋₄ alkyl-NR₂₇ₐR_{27b} and CONR₂₇ₐR_{27b};
one of R^{14a} and R^{14b} is H; and
the 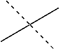
indicates the site of attachment of the chemical linking moiety coupling the PTM to the ULM.

11. The compound of claim 9, wherein the ULM is of the formula: 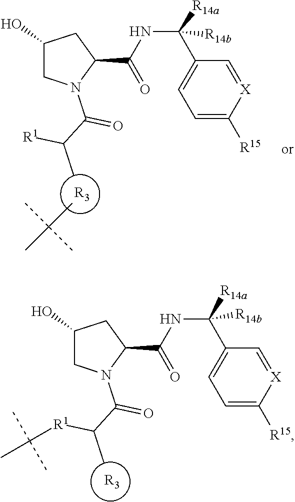
or a pharmaceutically acceptable salt thereof, wherein:
X is CH or N; and
at least one of: one of R₁₄ₐ and R_{14b} is H, C₁₋₆ alkyl, C₁₋₆ haloalkyl, optionally substitute C₁₋₄ alkylamine, C₁₋₆ alkoxy, (CH₂)_{q}C₁₋₆ alkoxy, (CH₂)_{q}OH, (CH₂)_{q}NR₂₇ₐR_{27b}, C₃₋₆ cycloalkyl, or NR₂₇ₐR_{27b}; and one of R^{14a} and R^{14b} is H;
q is 1, 2, 3 or 4; and
the 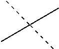
indicates the site of attachment of the chemical linking moiety coupling the PTM to the ULM.

12. The compound of claim 11, or a pharmaceutically acceptable salt thereof, wherein R₁ is C₁₋₆ alkyl.

13. The compound of any one of claims 1-12, or a pharmaceutically acceptable salt thereof, wherein one of R₁₄ₐ and R_{14b} is H, C₁₋₄ alkyl, C₁₋₄ haloalkyl, C₁₋₄ alkoxy, optionally substituted C₁₋₄ alkylamine, (CH₂)_{q}OH, (CH₂)_{q}NR₂₇ₐR_{27b}, C₃₋₆ cycloalkyl, or NR₂₇ₐR_{27b}; and one of R₁₄ₐ and R_{14b} is H.

14. The compound of any one of claims 1-13, wherein each R₂₇ₐ and R_{27b} is independently H or C₁₋₄ alkyl.

15. The compound of any one of claims 1-14, wherein q is 1 or 2.

16. The compound of any one of claims 5-14, or a pharmaceutically acceptable salt thereof, wherein:
R₂₈ is C₁₋₆ alkyl, C₃₋₆ cycloalkyl, C₁₋₆ haloalkyl, (CH₂)_{q}OC₁₋₆alkyl, (CH₂)_{q}OH, (CH₂)_{q}NR₂₇ₐR_{27b}, (CH₂)_{q}NHCOC₁₋₆ alkyl, or 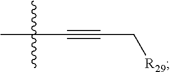
R₂₉ is H, C₁₋₆ alkyl, NR₂₇ₐR_{27b} or _{q}NHCOC₁₋₆ alkyl; and
q is 1 or 2.

17. The compound of any one of claims 7-16, or a pharmaceutically acceptable salt thereof, wherein R³ is isoxazolyl, 4-chloroisoxazolyl, 4-fluoroisoxazolyl, or pyrazolyl.

18. The compound of claim 3 or 11, or a pharmaceutically acceptable salt thereof, wherein X is CH.

19. The compound of claim 1 or 2, wherein the ULM has a chemical structure selected from: 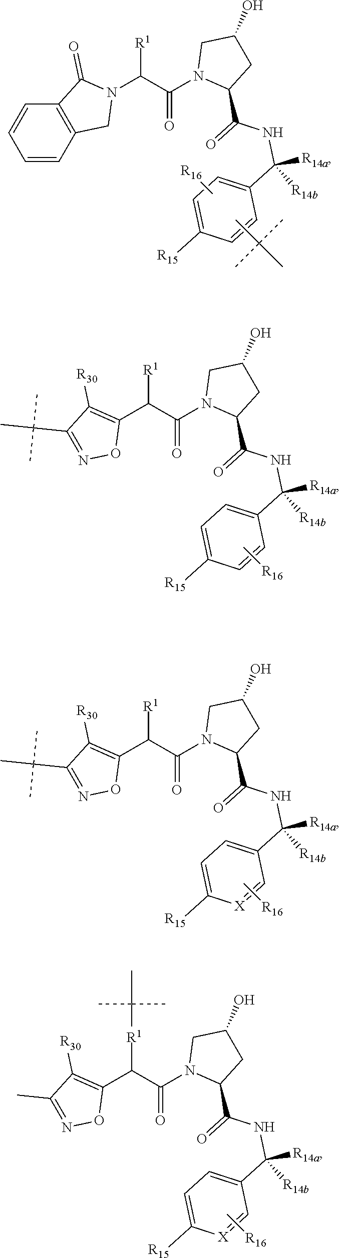
or a pharmaceutically acceptable salt thereof, wherein:
X is CH or N;
R₃₀ is H, F or Cl;
R₁ is a C₁₋₆ alkyl;
one of R₁₄ₐ and R_{14b} is an H, methyl, C1 fluoroalkyl, CHF₂, CF₃, and the other is an H;
R₁₅ is selected from: cyano, halogen (e.g., F or C₁), 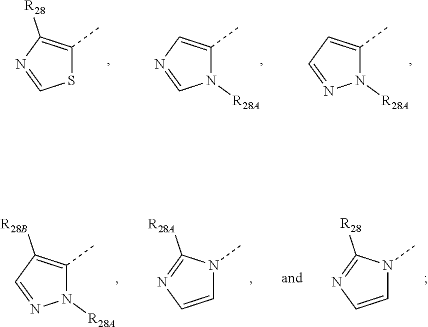
each R¹⁶ is individually selected from H, C₁₋₄alkyl, fluoro, chloro, NH₂, CN, and C₁₋₄alkoxy; and
R_{28A} is selected from H or methyl;
R_{28B} is selected from H, methyl, fluoro, and chloro;
R₂₈ is selected from H, methyl, CH₂N(Me)₂, CH₂OH, CH₂O(C₁₋₄alkyl), CH₂NHC(O)C₁₋₄alkyl, NH₂, 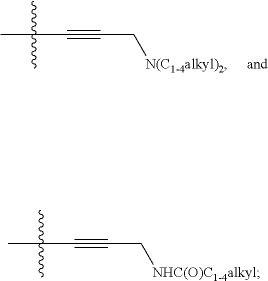
the 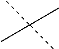
indicates the site of attachment of the chemical linking moiety coupling the PTM to the ULM.

20. The compound of claim 1 or 2, wherein the ULM is selected from: 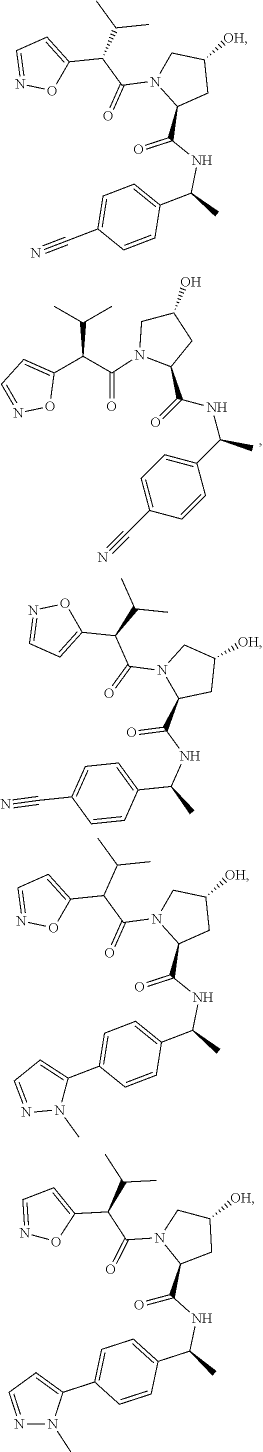 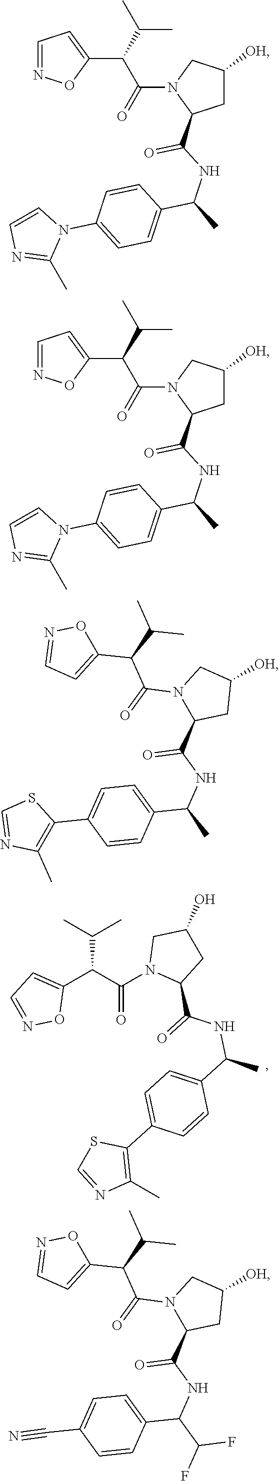 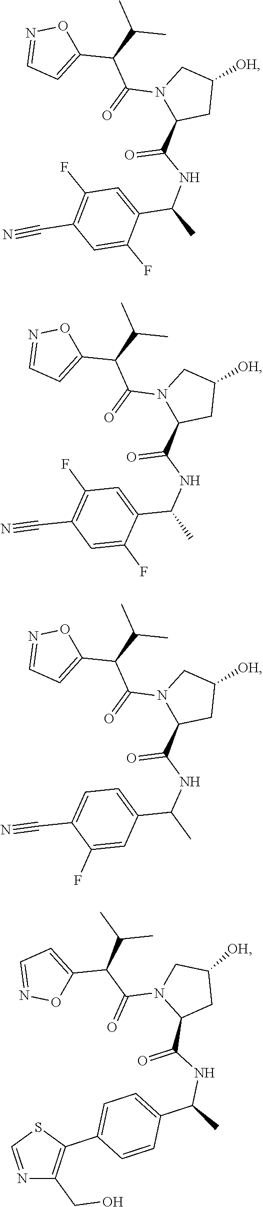 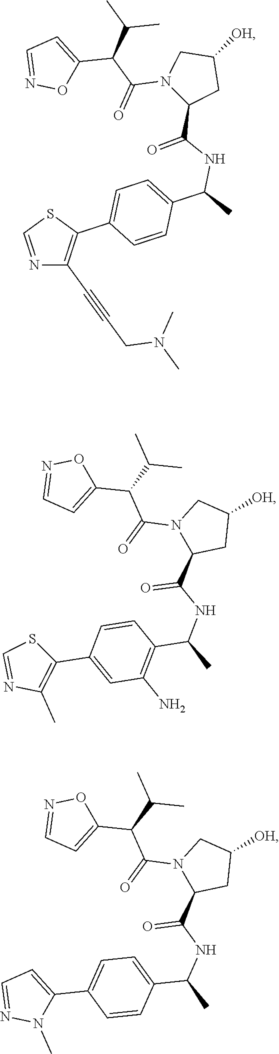 [SMILES: CC(C)[C@@H](C(=O)N1C[C@H](O)C[C@H]1C(=O)N[C@@H](C)c1ccc(-c2scnc2C#CCN(C)C)cc1)c1ccno1 ; Cc1ncsc1-c1ccc([C@H](C)NC(=O)[C@@H]2C[C@@H](O)CN2C(=O)[C@H](c2ccno2)C(C)C)c(N)c1 ; CC(C)[C@@H](C(=O)N1C[C@H](O)C[C@H]1C(=O)N[C@@H](C)c1ccc(-c2ccnn2C)cc1)c1ccno1] 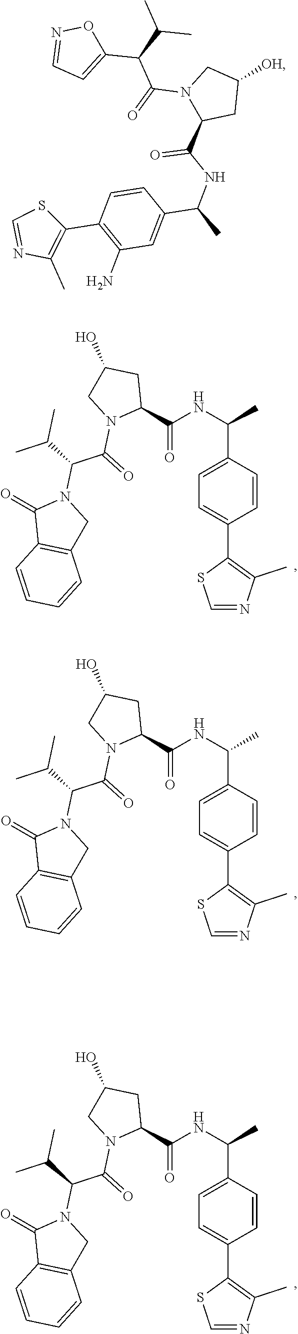 [SMILES: Cc1ncsc1-c1ccc([C@H](C)NC(=O)[C@@H]2C[C@@H](O)CN2C(=O)[C@@H](c2ccno2)C(C)C)cc1N ; Cc1ncsc1-c1ccc([C@H](C)NC(=O)[C@@H]2C[C@@H](O)CN2C(=O)[C@@H](C(C)C)N2Cc3ccccc3C2=O)cc1 ; Cc1ncsc1-c1ccc([C@@H](C)NC(=O)[C@@H]2C[C@@H](O)CN2C(=O)[C@@H](C(C)C)N2Cc3ccccc3C2=O)cc1 ; Cc1ncsc1-c1ccc([C@H](C)NC(=O)[C@@H]2C[C@@H](O)CN2C(=O)[C@H](C(C)C)N2Cc3ccccc3C2=O)cc1] 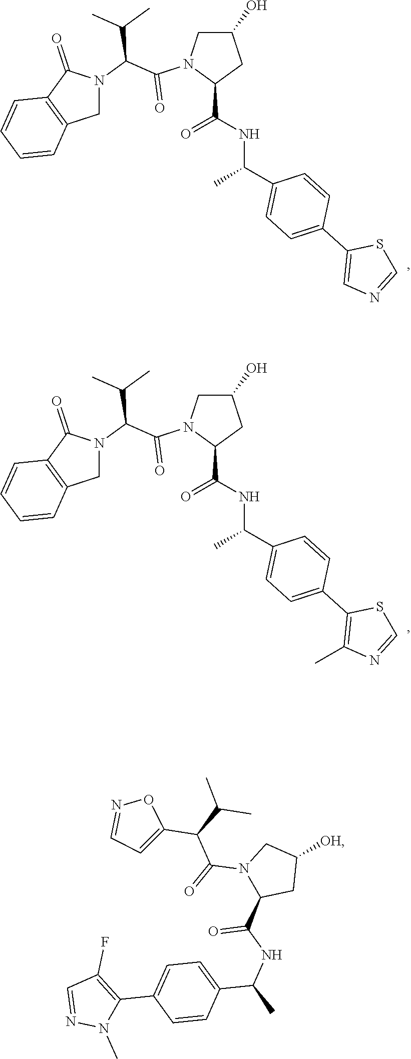 [SMILES: CC(C)[C@@H](C(=O)N1C[C@H](O)C[C@H]1C(=O)N[C@@H](C)c1ccc(-c2cncs2)cc1)N1Cc2ccccc2C1=O ; Cc1ncsc1-c1ccc([C@H](C)NC(=O)[C@@H]2C[C@@H](O)CN2C(=O)[C@H](C(C)C)N2Cc3ccccc3C2=O)cc1 ; CC(C)[C@@H](C(=O)N1C[C@H](O)C[C@H]1C(=O)N[C@@H](C)c1ccc(-c2c(F)cnn2C)cc1)c1ccno1] 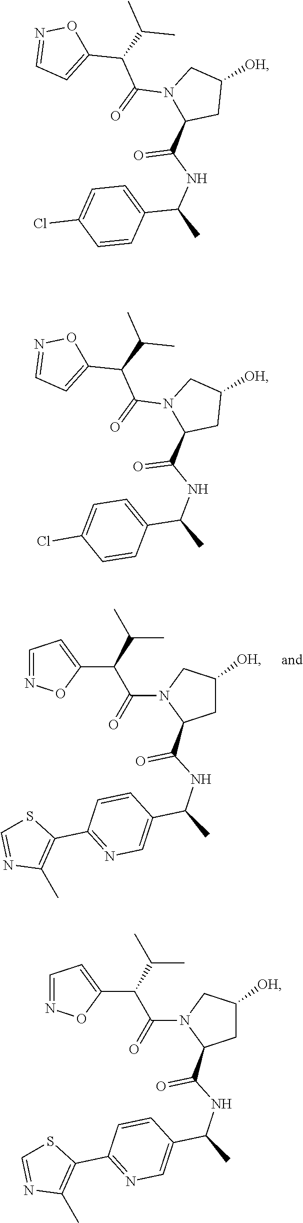 [SMILES: CC(C)[C@H](C(=O)N1C[C@H](O)C[C@H]1C(=O)N[C@@H](C)c1ccc(Cl)cc1)c1ccno1 ; CC(C)[C@@H](C(=O)N1C[C@H](O)C[C@H]1C(=O)N[C@@H](C)c1ccc(Cl)cc1)c1ccno1 ; Cc1ncsc1-c1ccc([C@H](C)NC(=O)[C@@H]2C[C@@H](O)CN2C(=O)[C@@H](c2ccno2)C(C)C)cn1 ; "and" ; Cc1ncsc1-c1ccc([C@H](C)NC(=O)[C@@H]2C[C@@H](O)CN2C(=O)[C@H](c2ccno2)C(C)C)cn1]
or a pharmaceutically acceptable salt thereof.

21. The compound of claim 1 or 2, wherein the ULM is selected from: 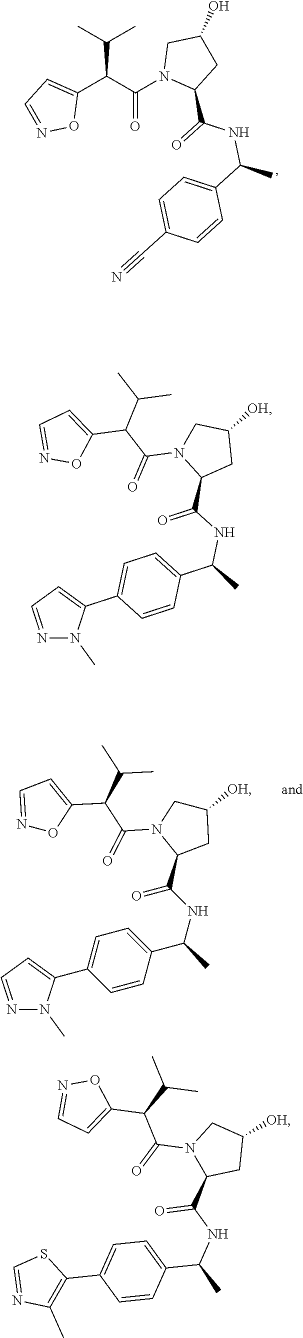 [SMILES: CC(C)[C@@H](C(=O)N1C[C@H](O)C[C@H]1C(=O)N[C@@H](C)c1ccc(C#N)cc1)c1ccno1 ; CC(C)C(C(=O)N1C[C@H](O)C[C@H]1C(=O)N[C@@H](C)c1ccc(-c2ccnn2C)cc1)c1ccno1 ; CC(C)[C@@H](C(=O)N1C[C@H](O)C[C@H]1C(=O)N[C@@H](C)c1ccc(-c2ccnn2C)cc1)c1ccno1 ; "and" ; Cc1ncsc1-c1ccc([C@H](C)NC(=O)[C@@H]2C[C@@H](O)CN2C(=O)[C@@H](c2ccno2)C(C)C)cc1]
or pharmaceutically acceptable salt thereof.

22. The compound according to any of claims 1-21, wherein the chemical linking moiety (L) is selected from the group consisting of: 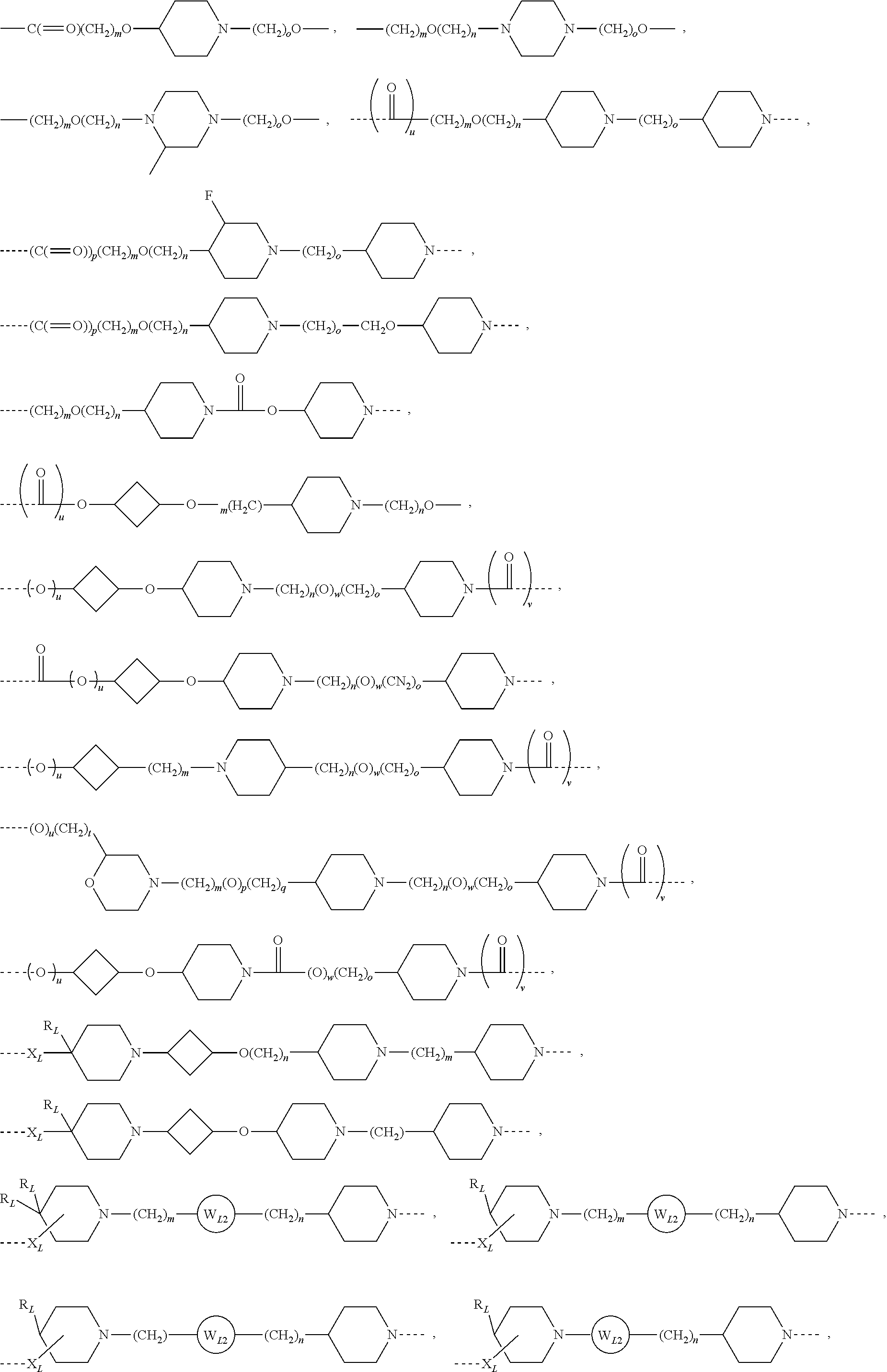 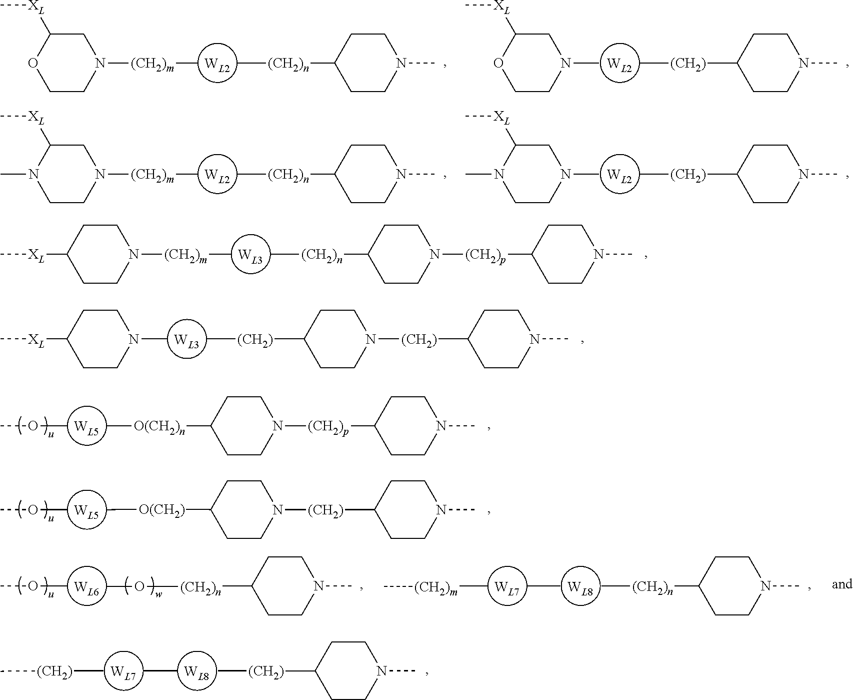
wherein:
each of m, n, o, p, q, and t is independently selected from the integers 0, 1, 2, 3 and 4 (preferably 0, 1, or 2); and
u, w, and v are each independently selected from integers 0 and 1.
X_{L} is —C(CH₂)—, —C(CH₃)H,— —CH₂—, —O—, C═O, or —NH—CH₂—;
R_{L} is H, OH, F, C₁, or methyl;
W_{L2} is selected from an optionally substituted 6-12 membered spirocycloalkylene or spirohetercyclylene (e.g. a 6-12 or 8-12 member spirocycloalkylene or spirohetercyclylene substituted with 0, 1, or 2 substituents selected from hydroxy, halogen, C₁₋₃ alkoxy, C₁₋₃ alkyl, C₁₋₃ haloalkyl, or amino);
W_{L3} is selected from an optionally substituted 6-12 membered spirocycloalkylene or spirohetercyclylene (e.g. a 6-12 or 8-12 member spirocycloalkylene or spirohetercyclylene substituted with 0, 1, or 2 substituents selected from hydroxy, halogen, C₁₋₃ alkoxy, C₁₋₃ alkyl, C₁₋₃ haloalkyl, or amino);
W_{L5} is selected from an optionally substituted 6-12 membered spirocycloalkylene or spirohetercyclylene (e.g. a 6-12 or 8-12 member spirocycloalkylene or spirohetercyclylene substituted with 0, 1, or 2 substituents selected from hydroxy, halogen, C₁₋₃ alkoxy, C₁₋₃ alkyl, C₁₋₃ haloalkyl, or amino);
W_{L6} is selected from an optionally substituted 6-12 membered spirocycloalkylene or spirohetercyclylene (e.g. a 6-12 or 8-12 member spirocycloalkylene or spirohetercyclylene substituted with 0, 1, or 2 substituents selected from hydroxy, halogen, C₁₋₃ alkoxy, C₁₋₃ alkyl, C₁₋₃ haloalkyl, or amino);
W_{L7} is selected from an optionally substituted 6-12 membered spirocycloalkylene or spirohetercyclylene (e.g. a 6-12 or 8-12 member spirocycloalkylene or spirohetercyclylene substituted with 0, 1, or 2 substituents selected from hydroxy, halogen, C₁₋₃ alkoxy, C₁₋₃ alkyl, C₁₋₃ haloalkyl, or amino);
W_{L8} is selected from an optionally substituted 6-12 membered spirocycloalkylene or spirohetercyclylene (e.g. a 6-12 or 8-12 member spirocycloalkylene or spirohetercyclylene substituted with 0, 1, or 2 substituents selected from hydroxy, halogen, C₁₋₃ alkoxy, C₁₋₃ alkyl, C₁₋₃ haloalkyl, or amino).

23. The compound according to claim 21, wherein each of m, n, o, p, q, and t is independently selected from the integers 0, 1, or 2.

24. The compound according to claim 22 or 23, wherein:
W_{L2} is selected from 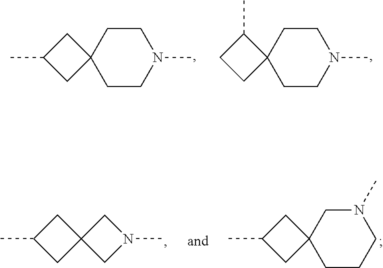
W_{L3} is 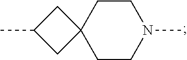
W_{L5} is selected from 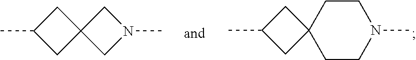
W_{L6} is selected from 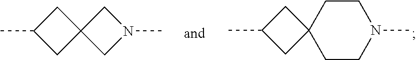
W_{L7} is selected from 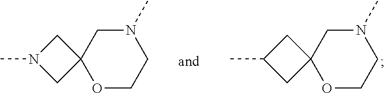
W_{L8} is selected from 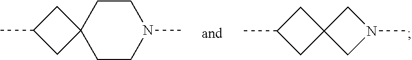
or
W_{L7} is selected from 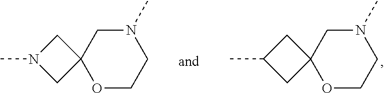
and/or W_{L8} is selected from

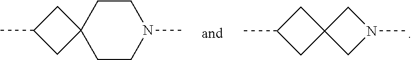

25. The compound according to any one of claims 1-24, wherein the chemical linking moiety (L) is selected from:

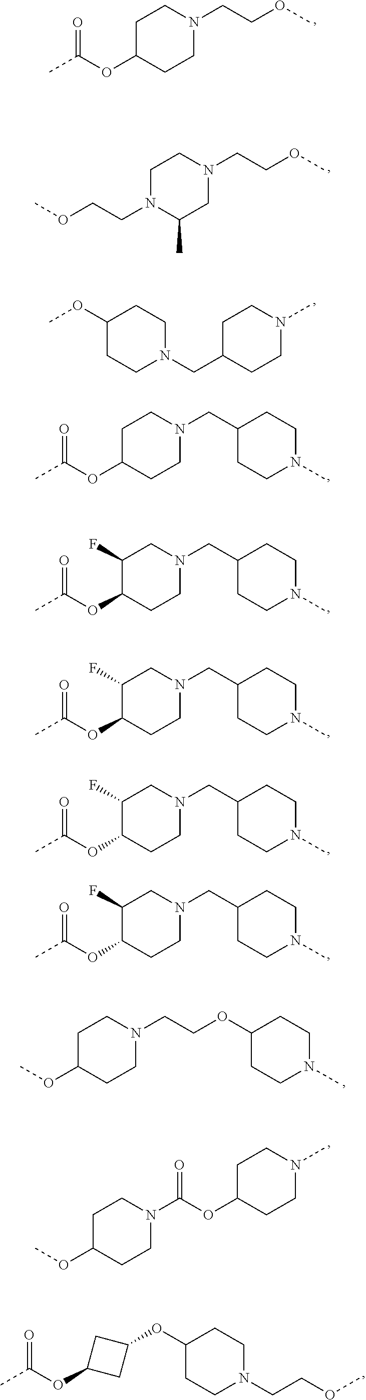

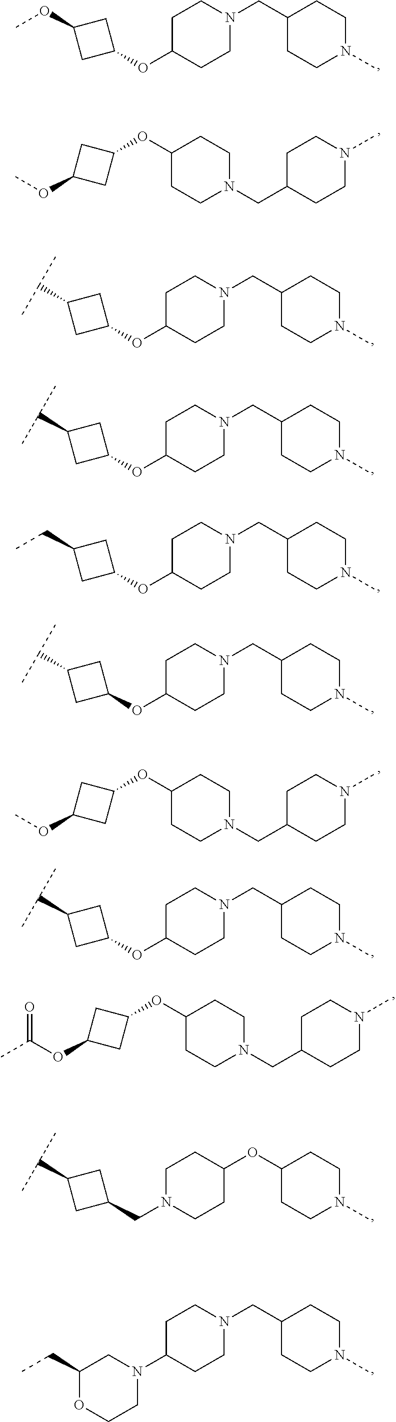

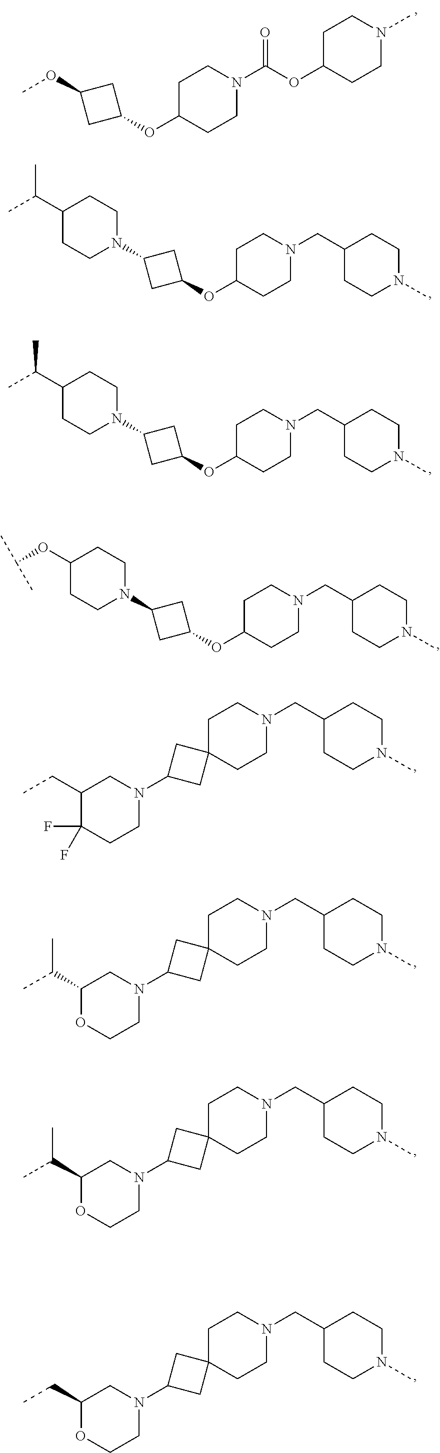

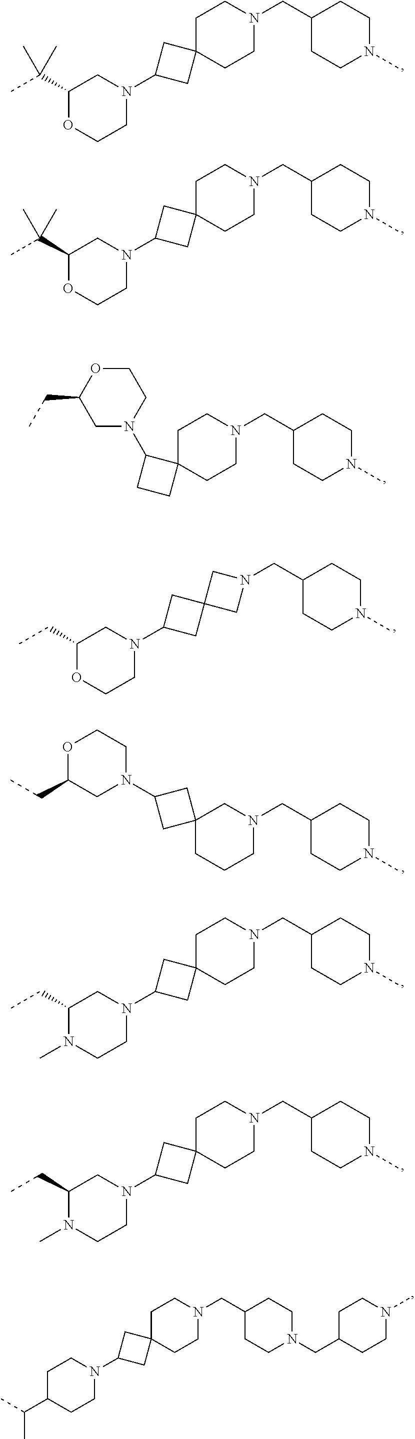

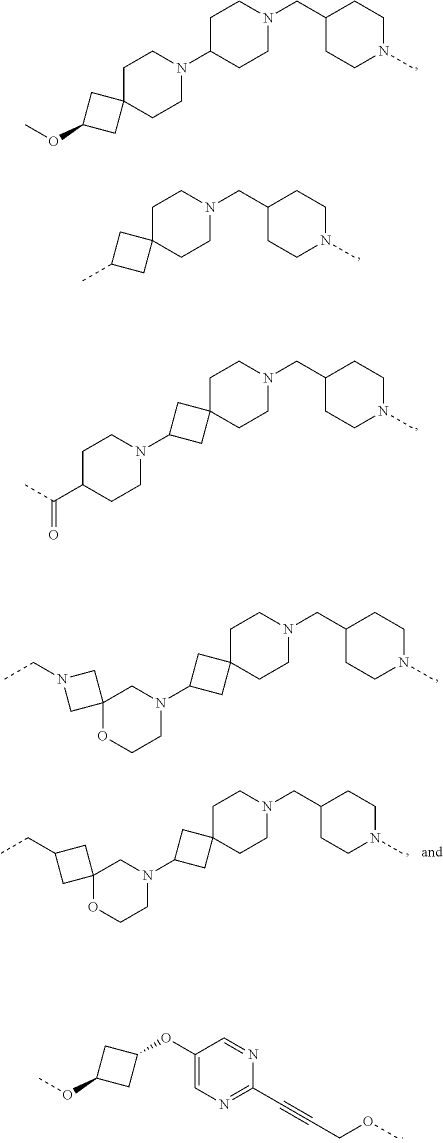

26. A compound selected from Table 1 (e.g., a compound selected from compounds 1-157).

27. The compound of claim 26 selected from: 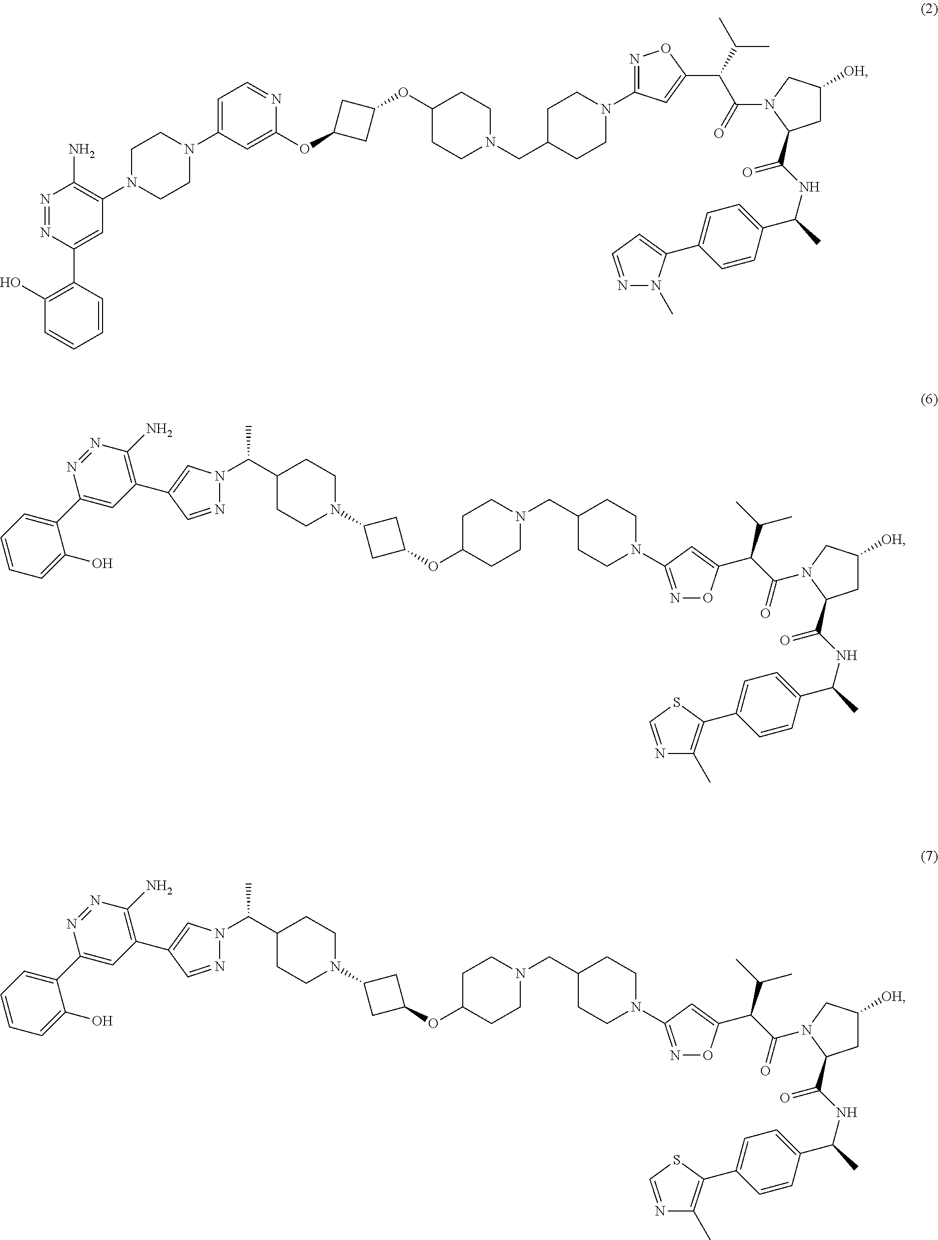 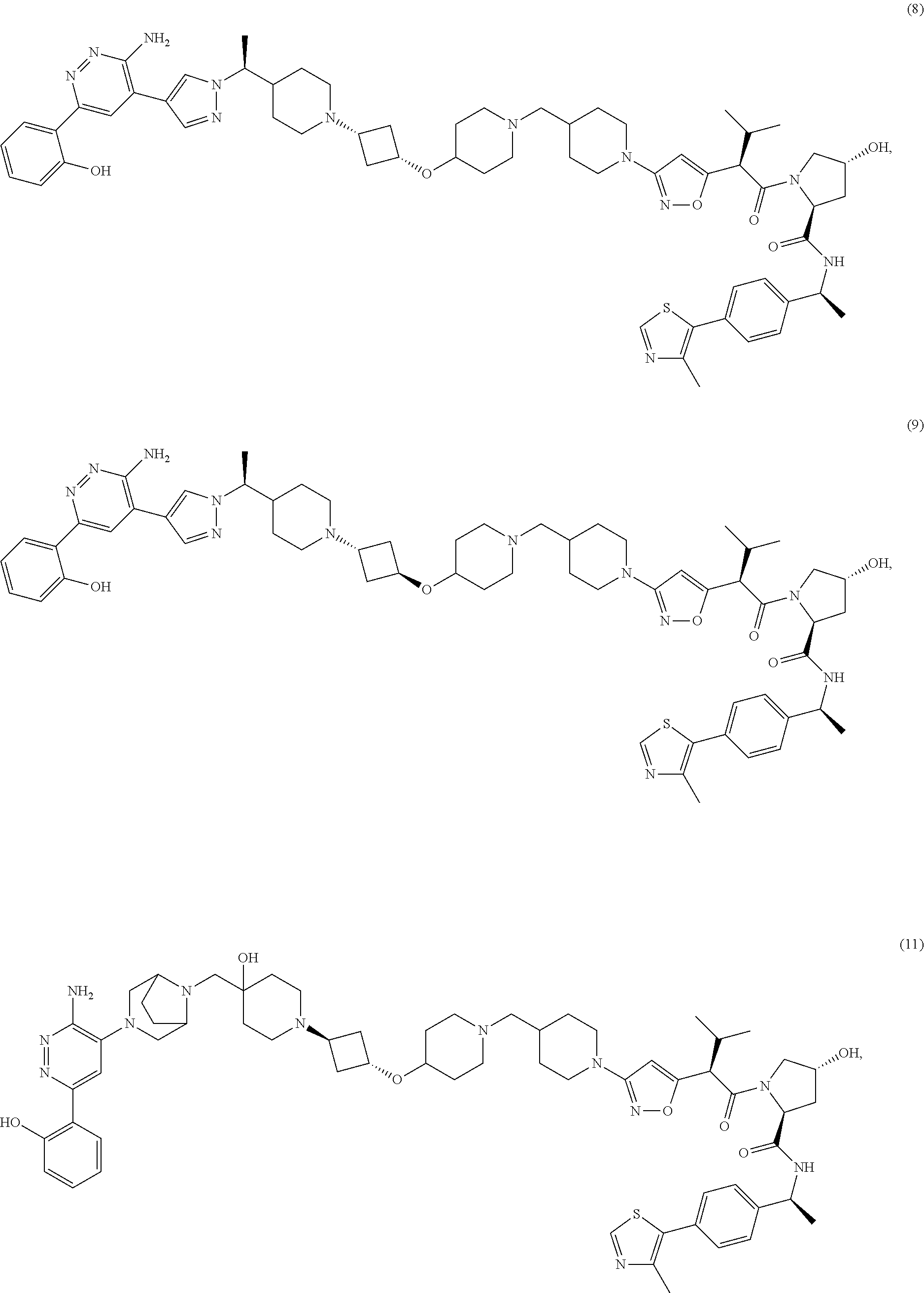 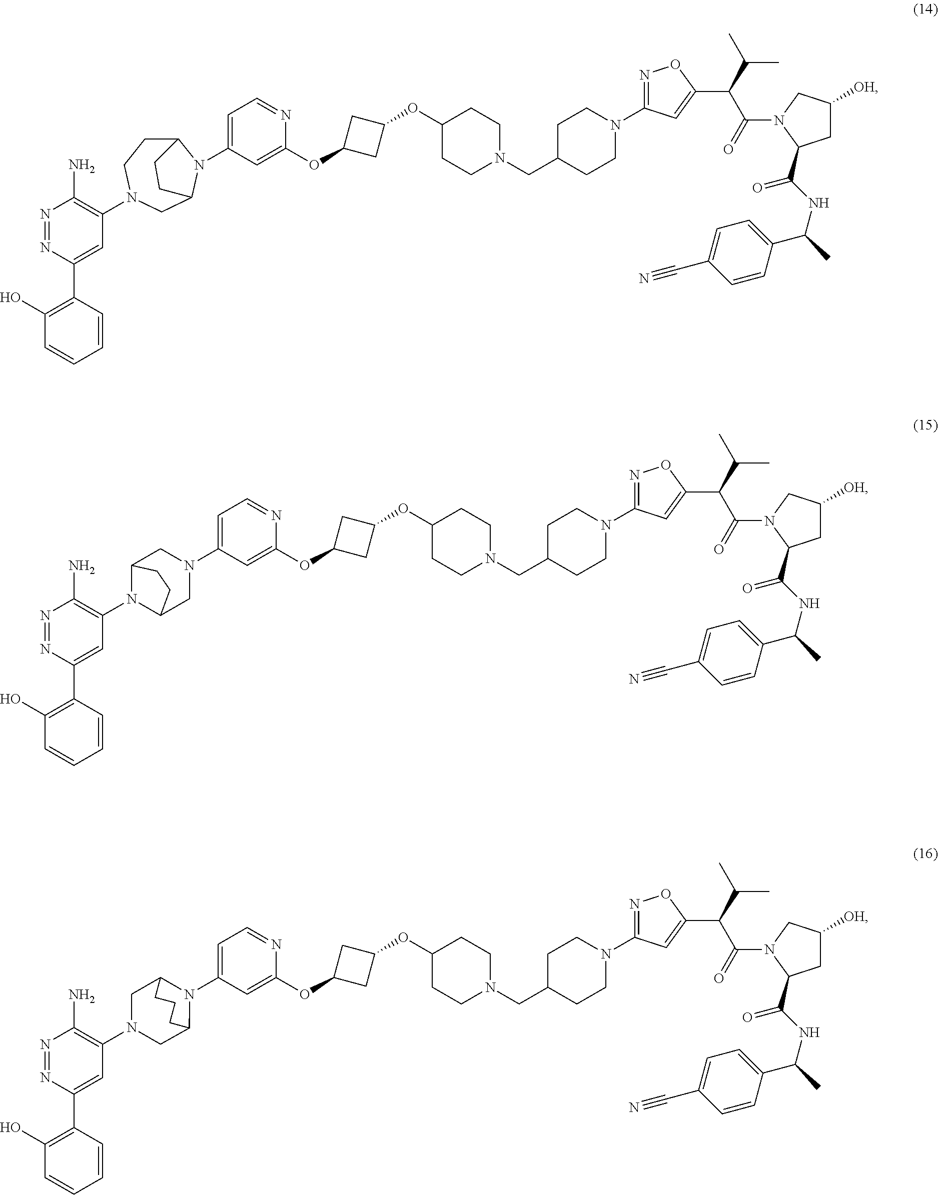 [SMILES: CC(C)[C@@H](C(=O)N1C[C@H](O)C[C@H]1C(=O)N[C@@H](C)c1ccc(C#N)cc1)c1cc(N2CCC(CN3CCC(O[C@H]4C[C@H](Oc5cc(N6C7CCC6CN(c6cc(-c8ccccc8O)nnc6N)CC7)ccn5)C4)CC3)CC2)no1 ; CC(C)[C@@H](C(=O)N1C[C@H](O)C[C@H]1C(=O)N[C@@H](C)c1ccc(C#N)cc1)c1cc(N2CCC(CN3CCC(O[C@H]4C[C@H](Oc5cc(N6CC7CCC(C6)N7c6cc(-c7ccccc7O)nnc6N)ccn5)C4)CC3)CC2)no1 ; CC(C)[C@@H](C(=O)N1C[C@H](O)C[C@H]1C(=O)N[C@@H](C)c1ccc(C#N)cc1)c1cc(N2CCC(CN3CCC(O[C@H]4C[C@H](Oc5cc(N6C7CCCC6CN(c6cc(-c8ccccc8O)nnc6N)C7)ccn5)C4)CC3)CC2)no1] 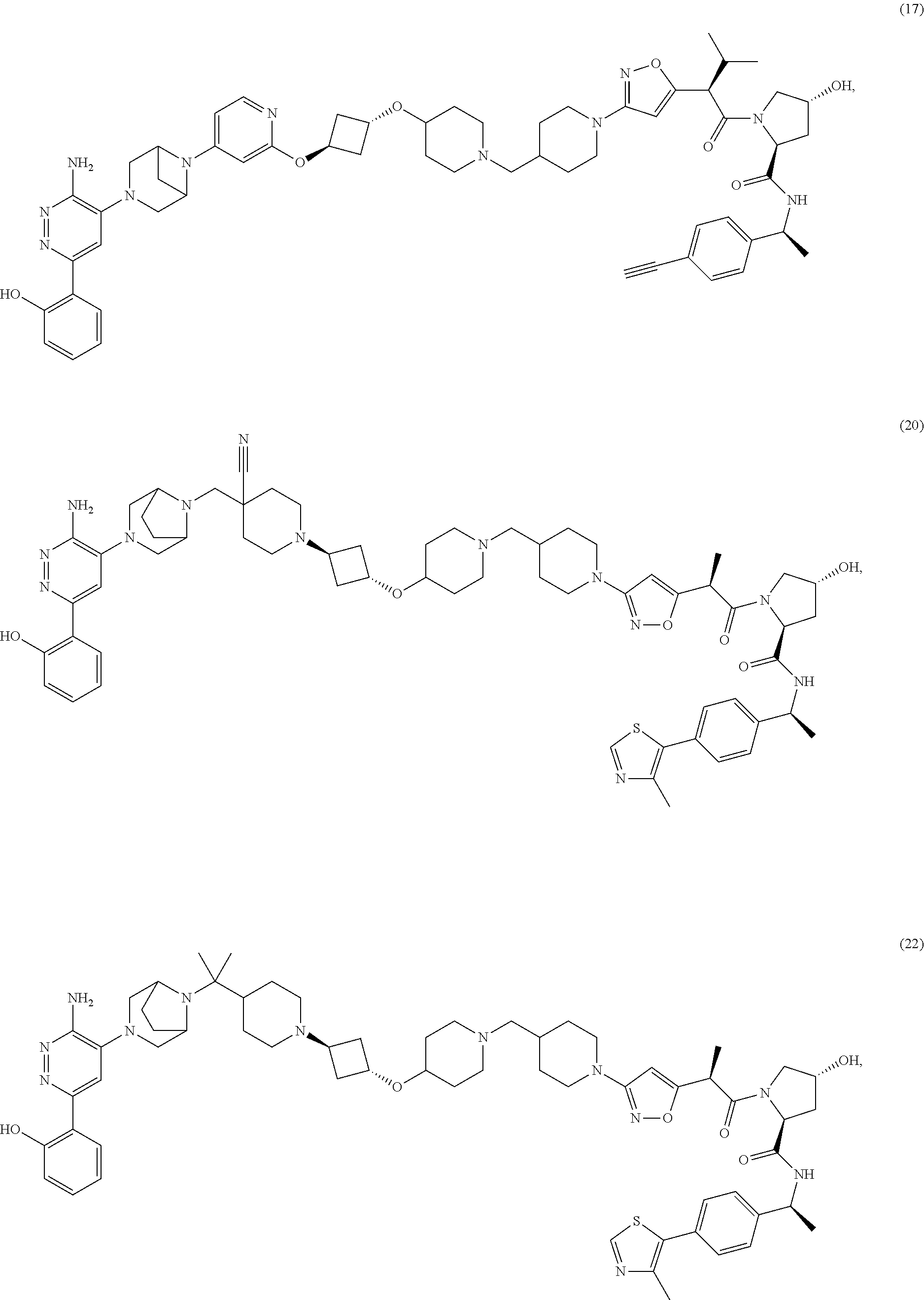 [SMILES: C#Cc1ccc([C@H](C)NC(=O)[C@@H]2C[C@@H](O)CN2C(=O)[C@@H](c2cc(N3CCC(CN4CCC(O[C@H]5C[C@H](Oc6cc(N7C8CC7CN(c7cc(-c9ccccc9O)nnc7N)C8)ccn6)C5)CC4)CC3)no2)C(C)C)cc1 ; Cc1ncsc1-c1ccc([C@H](C)NC(=O)[C@@H]2C[C@@H](O)CN2C(=O)[C@H](C)c2cc(N3CCC(CN4CCC(O[C@H]5C[C@H](N6CCC(C#N)(CN7C8CCC7CN(c7cc(-c9ccccc9O)nnc7N)C8)CC6)C5)CC4)CC3)no2)cc1 ; Cc1ncsc1-c1ccc([C@H](C)NC(=O)[C@@H]2C[C@@H](O)CN2C(=O)[C@H](C)c2cc(N3CCC(CN4CCC(O[C@H]5C[C@H](N6CCC(C(C)(C)N7C8CCC7CN(c7cc(-c9ccccc9O)nnc7N)C8)CC6)C5)CC4)CC3)no2)cc1] 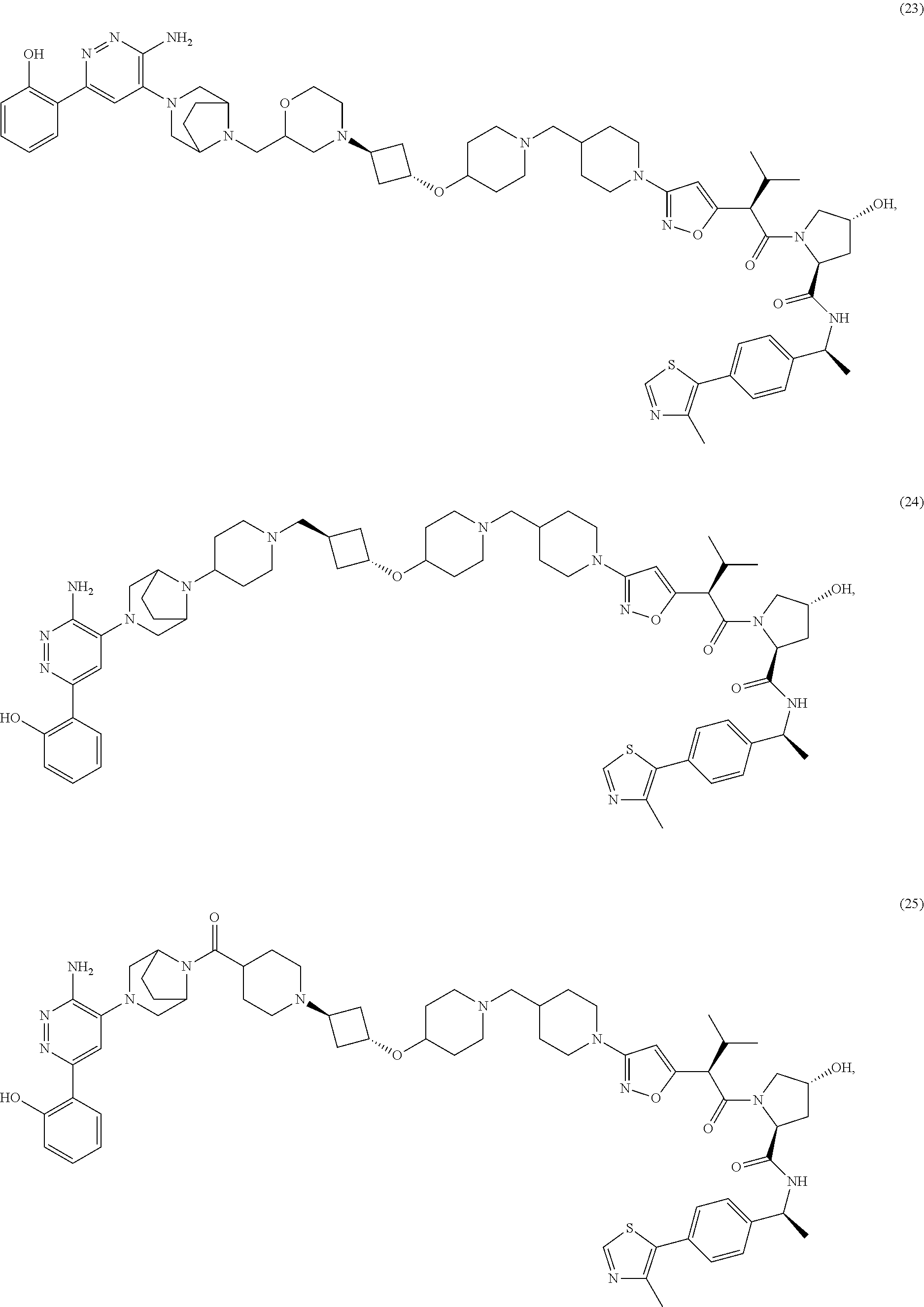 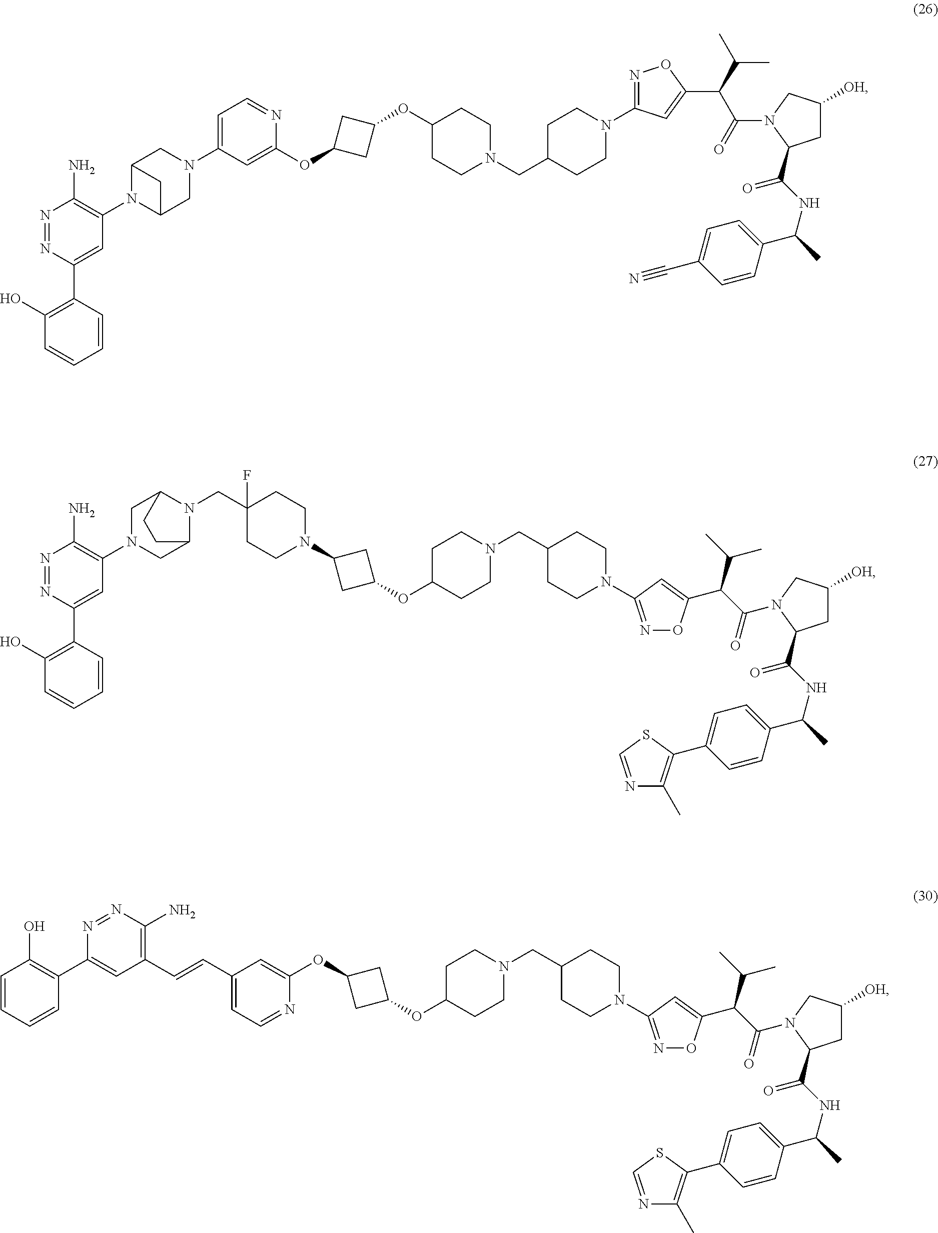 [SMILES: CC(C)[C@@H](C(=O)N1C[C@H](O)C[C@H]1C(=O)N[C@@H](C)c1ccc(C#N)cc1)c1cc(N2CCC(CN3CCC(O[C@H]4C[C@H](Oc5cc(N6CC7CC(C6)N7c6cc(-c7ccccc7O)nnc6N)ccn5)C4)CC3)CC2)no1 ; Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCC(F)(CN7C8CCC7CN(c7cc(-c9ccccc9O)nnc7N)C8)CC6)C5)CC4)CC3)no2)C(C)C)cc1 ; Cc1ncsc1-c1ccc([C@H](C)NC(=O)[C@@H]2C[C@@H](O)CN2C(=O)[C@@H](c2cc(N3CCC(CN4CCC(O[C@H]5C[C@H](Oc6cc(/C=C/c7cc(-c8ccccc8O)nnc7N)ccn6)C5)CC4)CC3)no2)C(C)C)cc1] 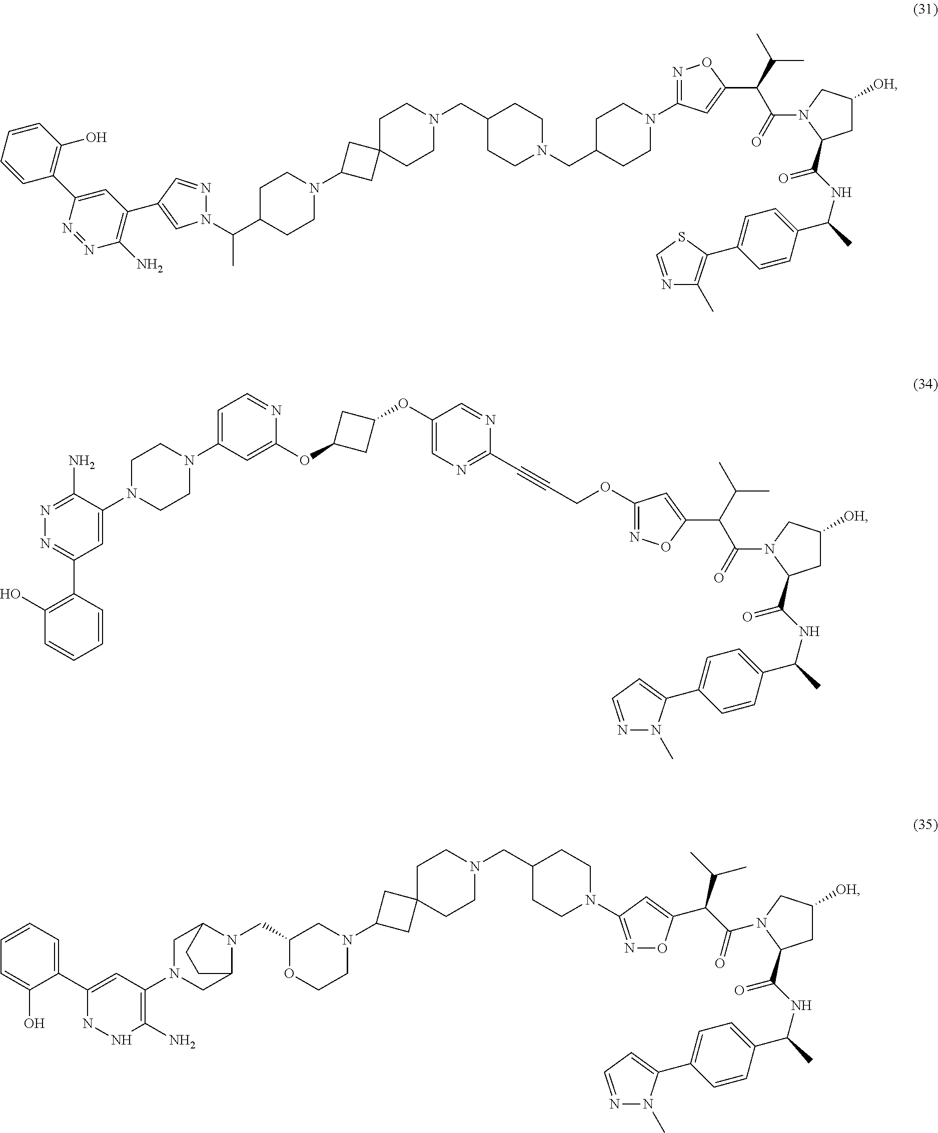 [SMILES: Cc1ncsc1-c1ccc([C@H](C)NC(=O)[C@@H]2C[C@@H](O)CN2C(=O)[C@@H](c2cc(N3CCC(CN4CCC(CN5CCC6(CC5)CC(N5CCC(C(C)n7cc(-c8cc(-c9ccccc9O)nnc8N)cn7)CC5)C6)CC4)CC3)no2)C(C)C)cc1 ; CC(C)C(C(=O)N1C[C@H](O)C[C@H]1C(=O)N[C@@H](C)c1ccc(-c2ccnn2C)cc1)c1cc(OCC#Cc2ncc(O[C@H]3C[C@H](Oc4cc(N5CCN(c6cc(-c7ccccc7O)nnc6N)CC5)ccn4)C3)cn2)no1 ; CC(C)[C@@H](C(=O)N1C[C@H](O)C[C@H]1C(=O)N[C@@H](C)c1ccc(-c2ccnn2C)cc1)c1cc(N2CCC(CN3CCC4(CC3)CC(N3CCO[C@H](CN5C6CCC5CN(C5=C(N)NNC(c7ccccc7O)=C5)C6)C3)C4)CC2)no1] 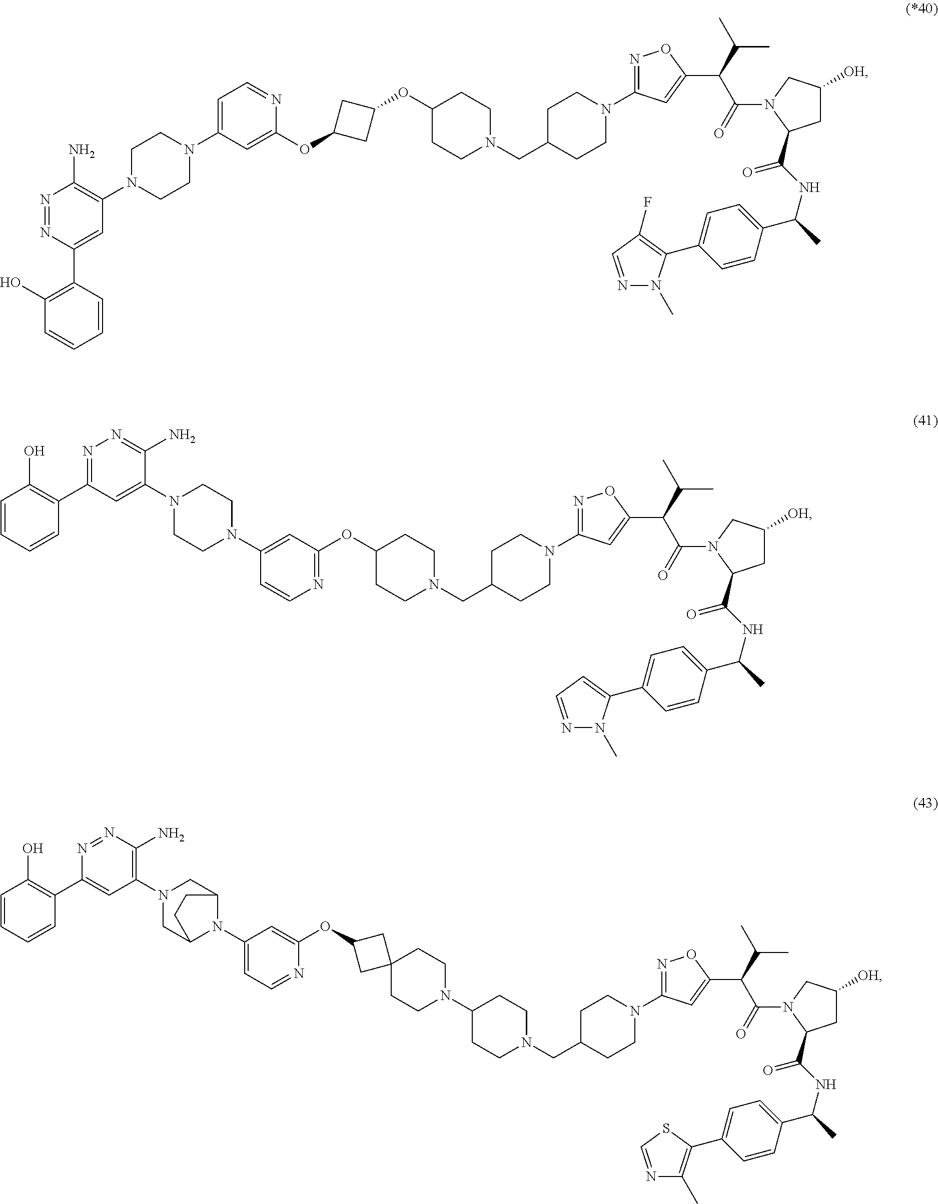 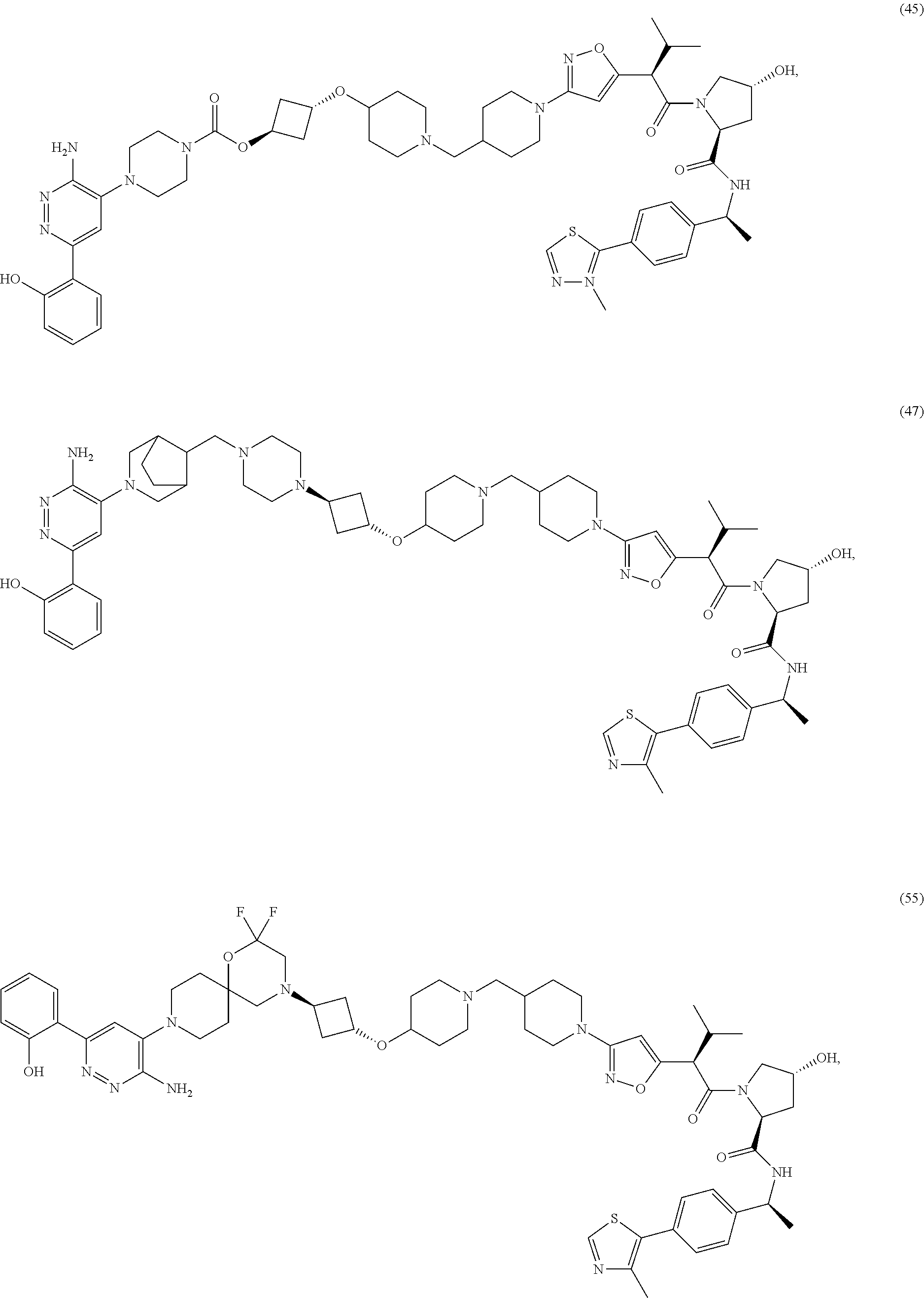 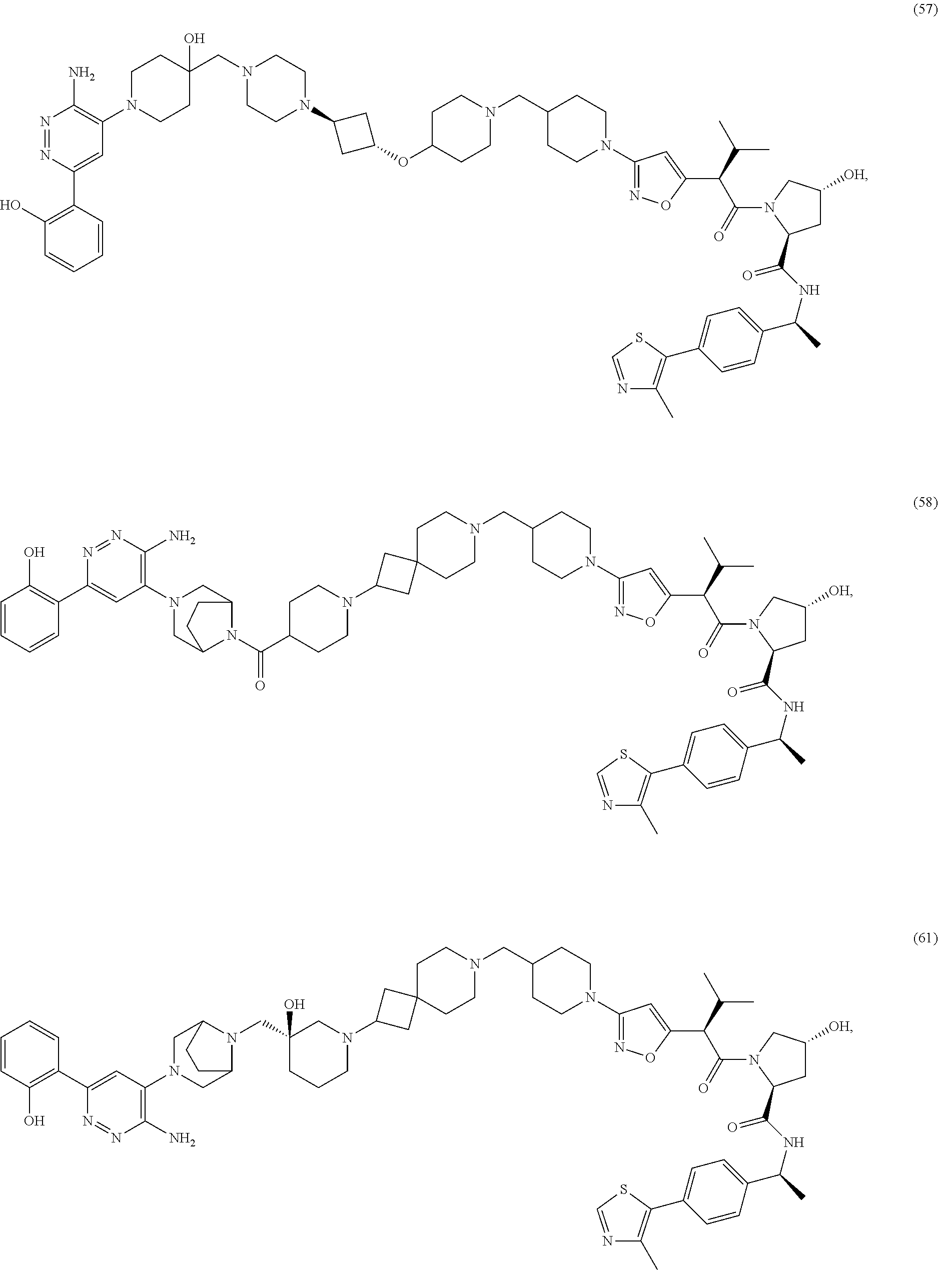 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCN(CC7(O)CCN(c8cc(-c9ccccc9O)nnc8N)CC7)CC6)C5)CC4)CC3)no2)C(C)C)cc1 ; Cc1ncsc1-c1ccc([C@H](C)NC(=O)[C@@H]2C[C@@H](O)CN2C(=O)[C@@H](c2cc(N3CCC(CN4CCC5(CC4)CC(N4CCC(C(=O)N6C7CCC6CN(c6cc(-c8ccccc8O)nnc6N)C7)CC4)C5)CC3)no2)C(C)C)cc1 ; Cc1ncsc1-c1ccc([C@H](C)NC(=O)[C@@H]2C[C@@H](O)CN2C(=O)[C@@H](c2cc(N3CCC(CN4CCC5(CC4)CC(N4CCC[C@](O)(CN6C7CCC6CN(c6cc(-c8ccccc8O)nnc6N)C7)C4)C5)CC3)no2)C(C)C)cc1] 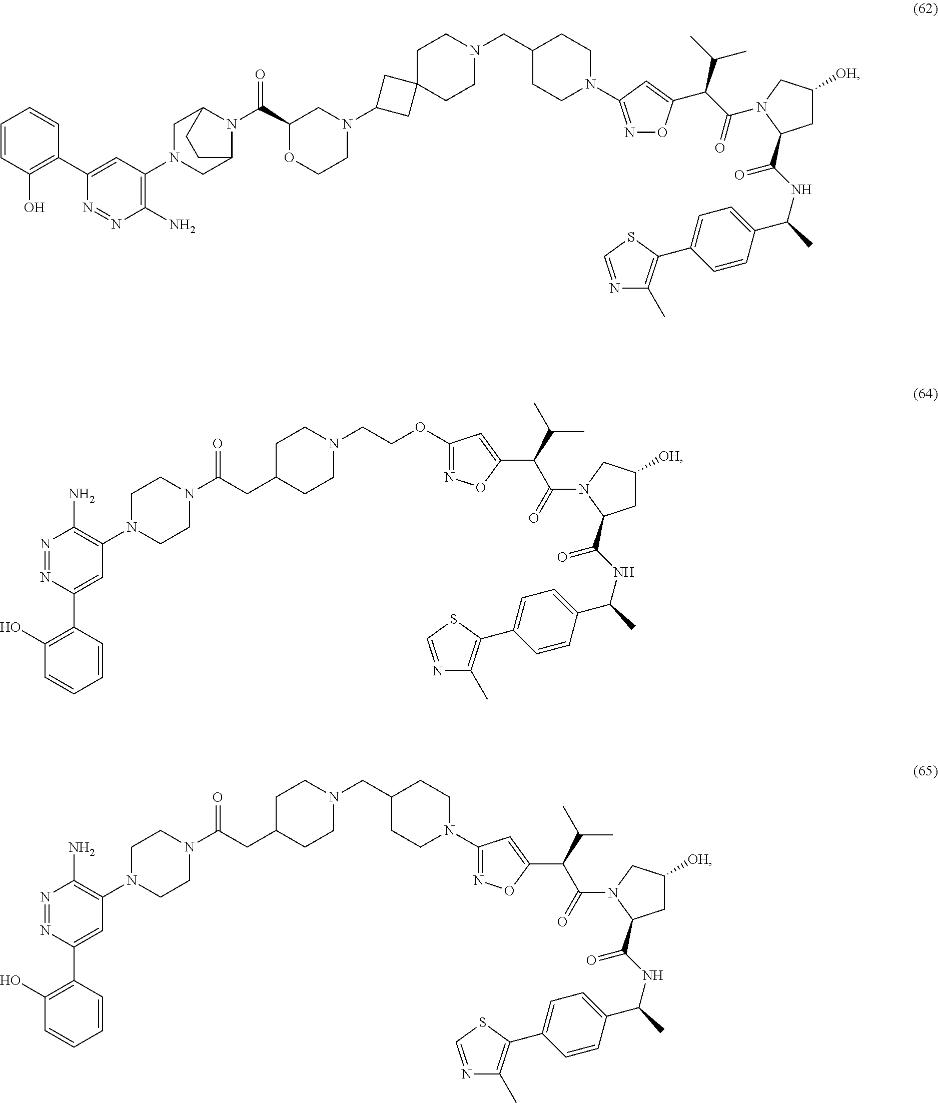 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(N4CCO[C@@H](C(=O)N6C7CCC6CN(c6cc(-c8ccccc8O)nnc6N)C7)C4)C5)CC3)no2)C(C)C)cc1 ; Cc1ncsc1-c1ccc([C@H](C)NC(=O)[C@@H]2C[C@@H](O)CN2C(=O)[C@@H](c2cc(OCCN3CCC(CC(=O)N4CCN(c5cc(-c6ccccc6O)nnc5N)CC4)CC3)no2)C(C)C)cc1 ; Cc1ncsc1-c1ccc([C@H](C)NC(=O)[C@@H]2C[C@@H](O)CN2C(=O)[C@@H](c2cc(N3CCC(CN4CCC(CC(=O)N5CCN(c6cc(-c7ccccc7O)nnc6N)CC5)CC4)CC3)no2)C(C)C)cc1] 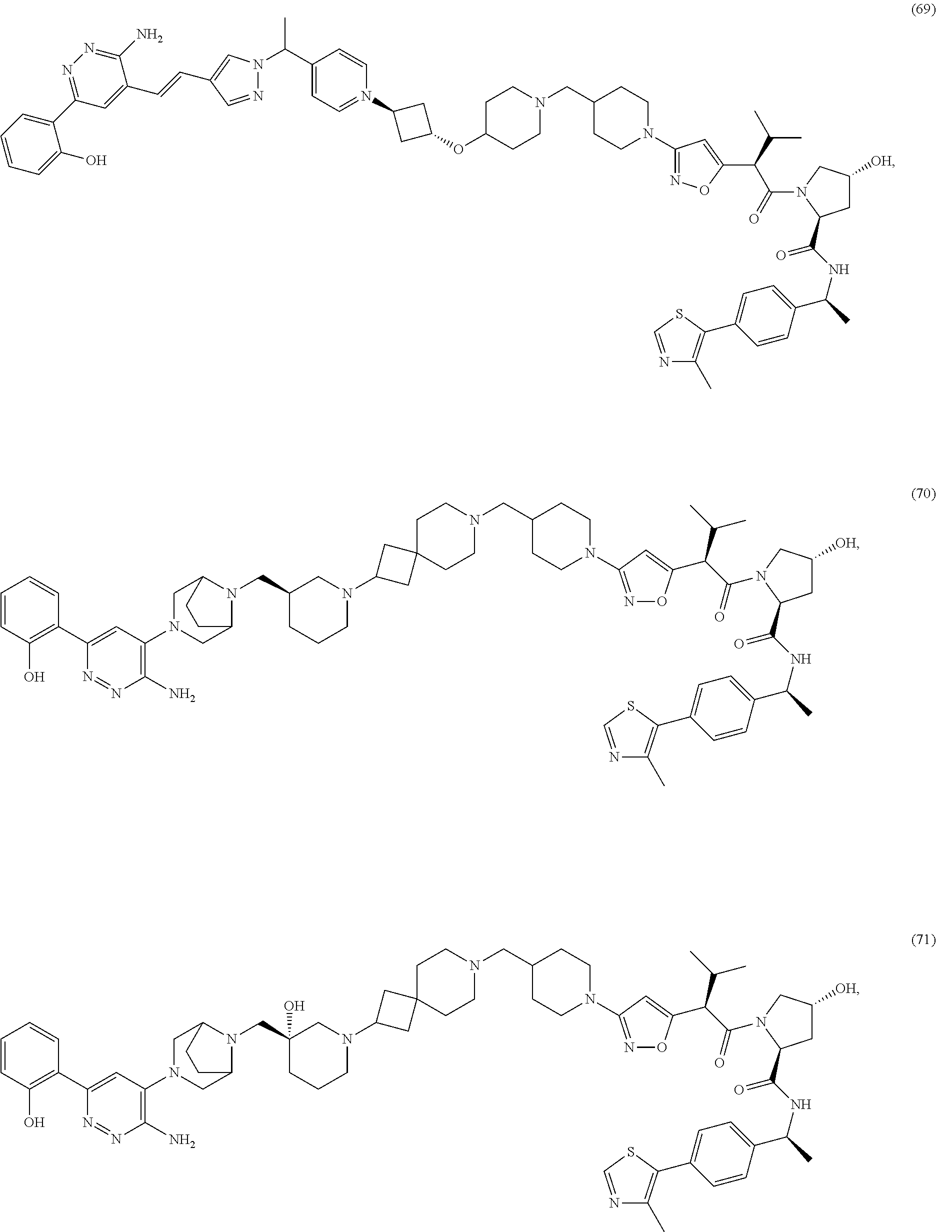 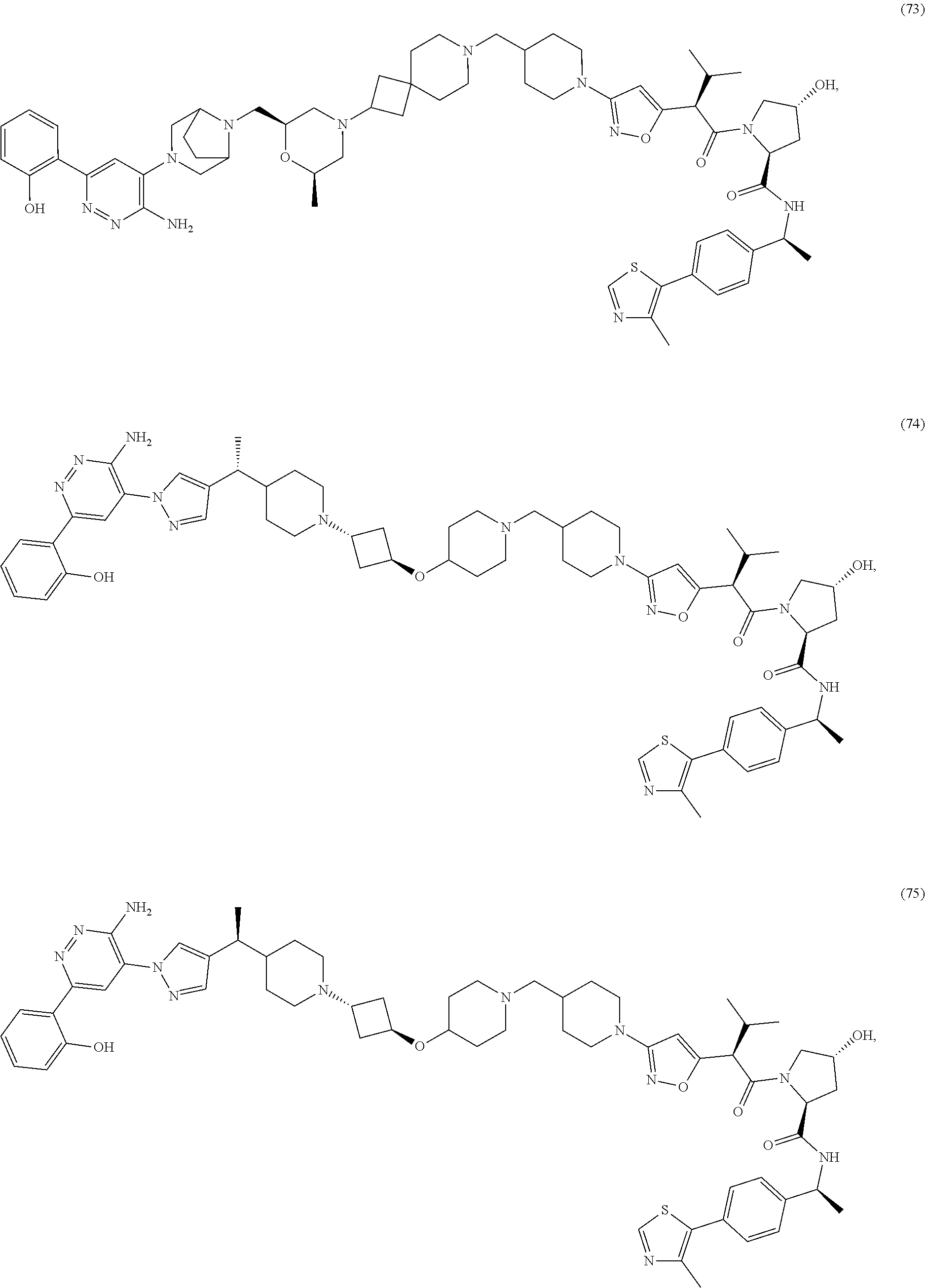 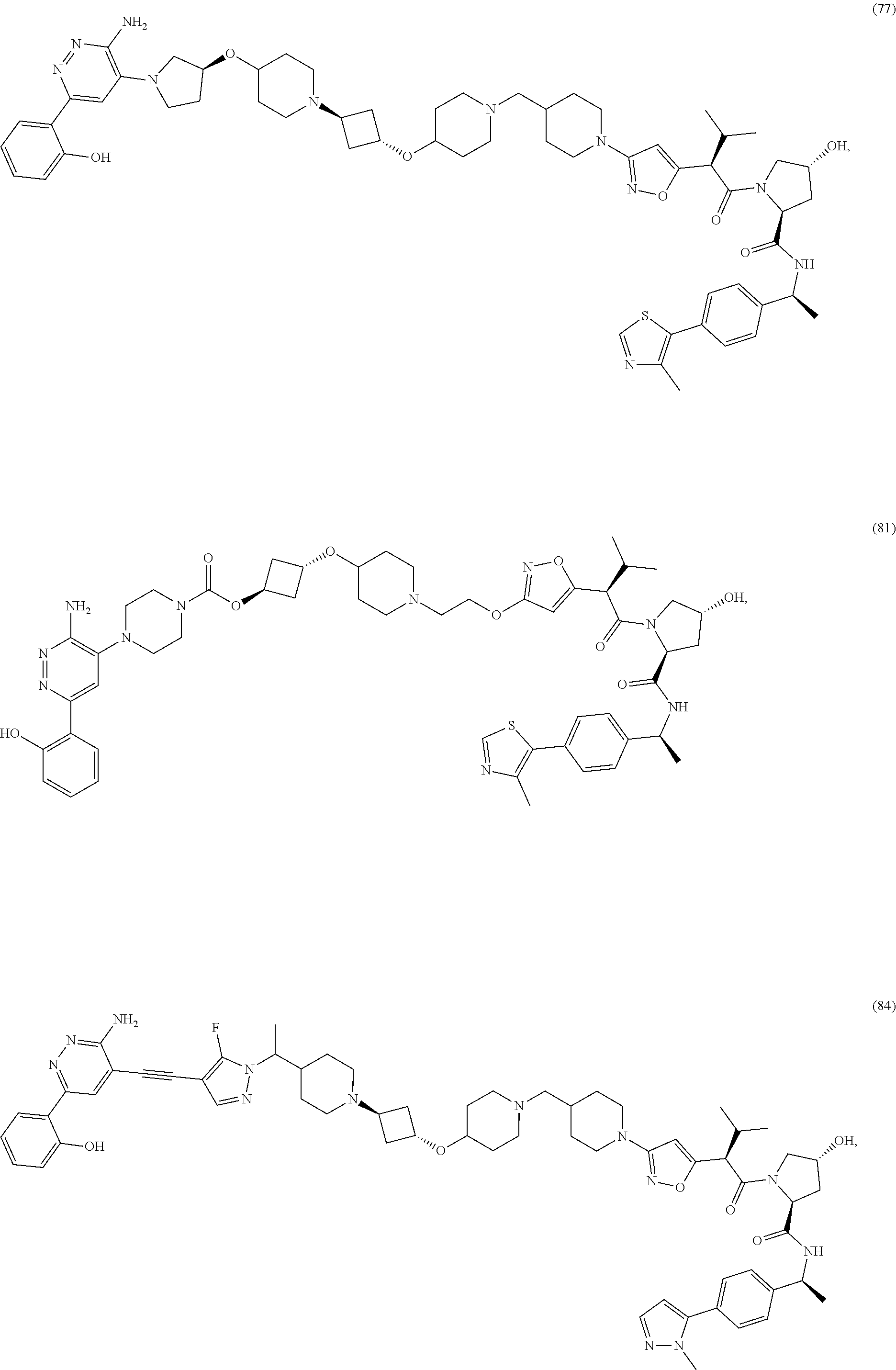 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCC(O[C@H]7CCN(c8cc(-c9ccccc9O)nnc8N)C7)CC6)C5)CC4)CC3)no2)C(C)C)cc1 ; Cc1ncsc1-c1ccc([C@H](C)NC(=O)[C@@H]2C[C@@H](O)CN2C(=O)[C@@H](c2cc(OCCN3CCC(O[C@H]4C[C@H](OC(=O)N5CCN(c6cc(-c7ccccc7O)nnc6N)CC5)C4)CC3)no2)C(C)C)cc1 ; CC(C)[C@@H](C(=O)N1C[C@H](O)C[C@H]1C(=O)N[C@@H](C)c1ccc(-c2ccnn2C)cc1)c1cc(N2CCC(CN3CCC(O[C@H]4C[C@H](N5CCC(C(C)n6ncc(C#Cc7cc(-c8ccccc8O)nnc7N)c6F)CC5)C4)CC3)CC2)no1] 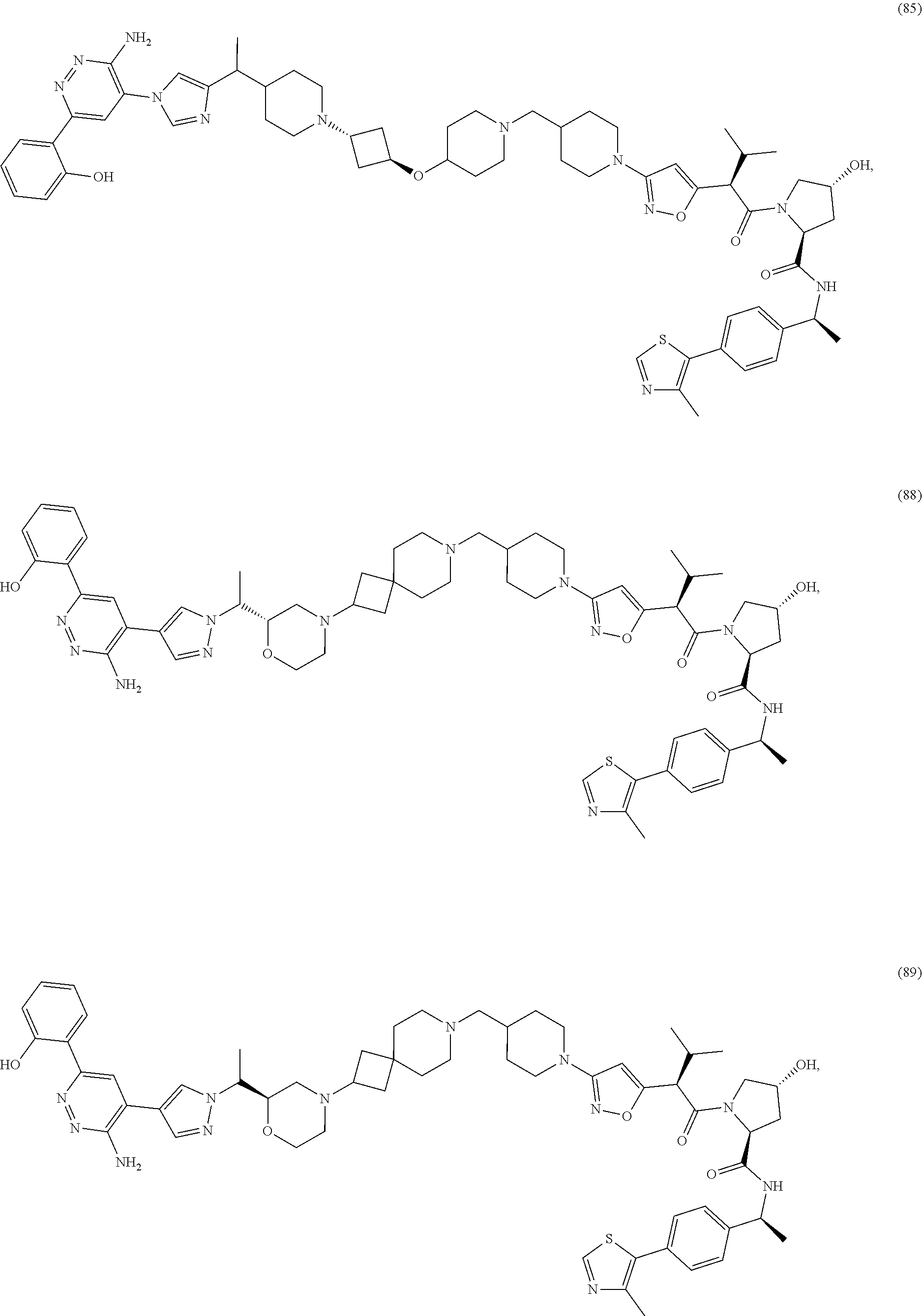 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCC(C(C)c7cn(-c8cc(-c9ccccc9O)nnc8N)cn7)CC6)C5)CC4)CC3)no2)C(C)C)cc1 ; Cc1ncsc1-c1ccc([C@H](C)NC(=O)[C@@H]2C[C@@H](O)CN2C(=O)[C@@H](c2cc(N3CCC(CN4CCC5(CC4)CC(N4CCO[C@H](C(C)n6cc(-c7cc(-c8ccccc8O)nnc7N)cn6)C4)C5)CC3)no2)C(C)C)cc1 ; Cc1ncsc1-c1ccc([C@H](C)NC(=O)[C@@H]2C[C@@H](O)CN2C(=O)[C@@H](c2cc(N3CCC(CN4CCC5(CC4)CC(N4CCO[C@@H](C(C)n6cc(-c7cc(-c8ccccc8O)nnc7N)cn6)C4)C5)CC3)no2)C(C)C)cc1] 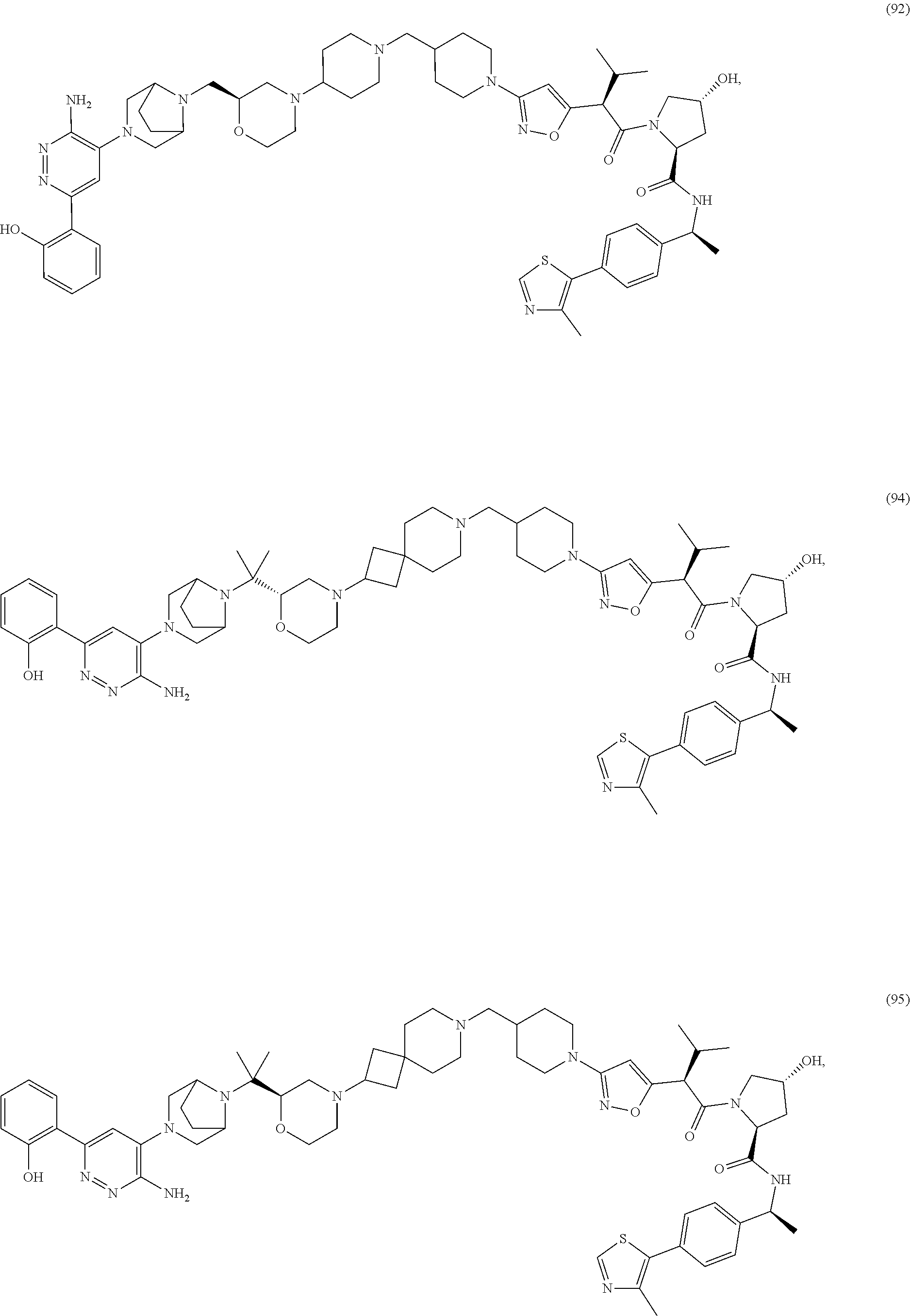 [SMILES: Cc1ncsc1-c1ccc([C@H](C)NC(=O)[C@@H]2C[C@@H](O)CN2C(=O)[C@@H](c2cc(N3CCC(CN4CCC(N5CCO[C@@H](CN6C7CCC6CN(c6cc(-c8ccccc8O)nnc6N)C7)C5)CC4)CC3)no2)C(C)C)cc1 ; Cc1ncsc1-c1ccc([C@H](C)NC(=O)[C@@H]2C[C@@H](O)CN2C(=O)[C@@H](c2cc(N3CCC(CN4CCC5(CC4)CC(N4CCO[C@H](C(C)(C)N6C7CCC6CN(c6cc(-c8ccccc8O)nnc6N)C7)C4)C5)CC3)no2)C(C)C)cc1 ; Cc1ncsc1-c1ccc([C@H](C)NC(=O)[C@@H]2C[C@@H](O)CN2C(=O)[C@@H](c2cc(N3CCC(CN4CCC5(CC4)CC(N4CCO[C@@H](C(C)(C)N6C7CCC6CN(c6cc(-c8ccccc8O)nnc6N)C7)C4)C5)CC3)no2)C(C)C)cc1] 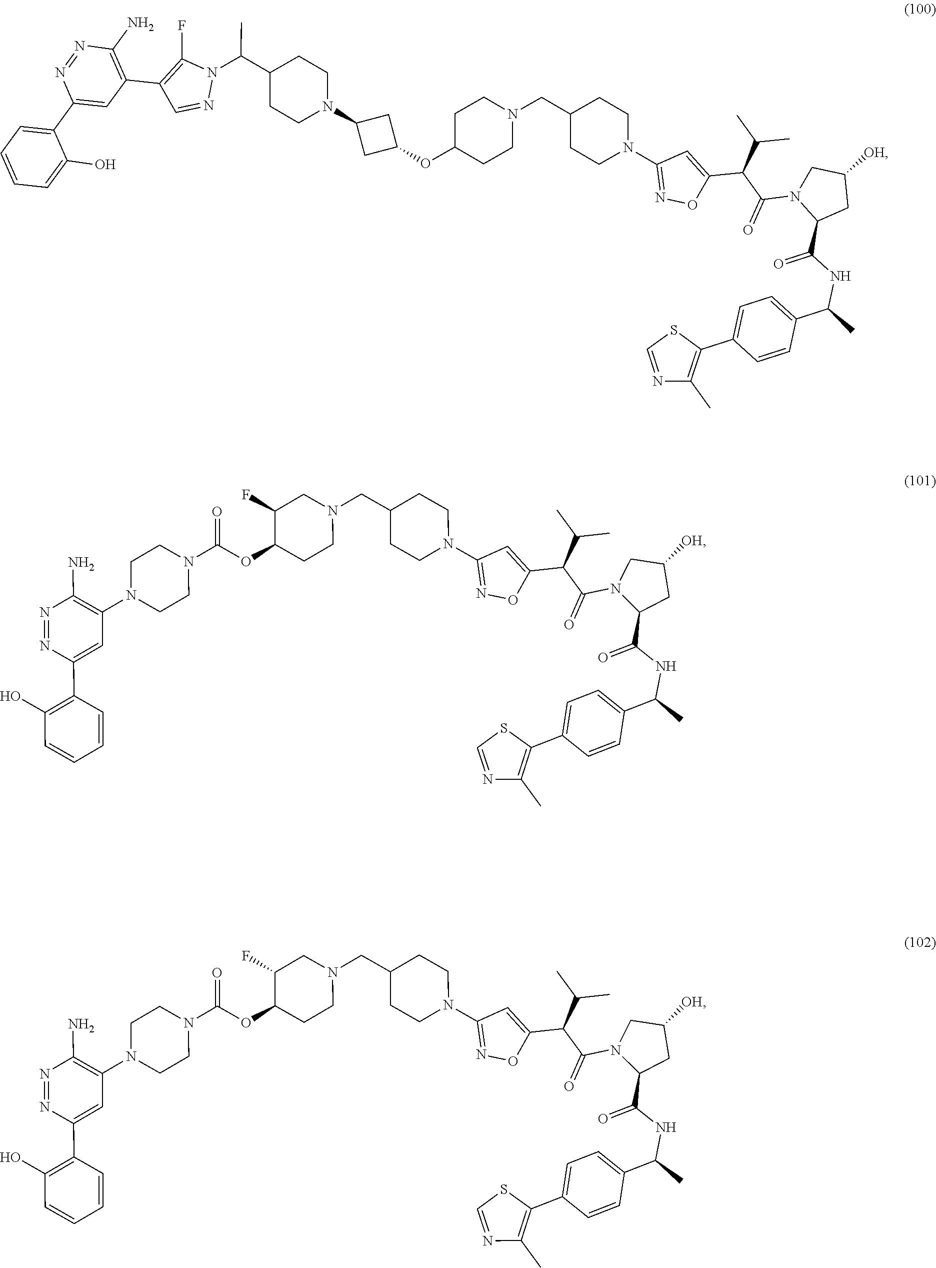 [SMILES: Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCC(C(C)n7ncc(-c8cc(-c9ccccc9O)nnc8N)c7F)CC6)C5)CC4)CC3)no2)C(C)C)cc1 ; Cc1ncsc1-c1ccc([C@H](C)NC(=O)[C@@H]2C[C@@H](O)CN2C(=O)[C@@H](c2cc(N3CCC(CN4CC[C@@H](OC(=O)N5CCN(c6cc(-c7ccccc7O)nnc6N)CC5)[C@@H](F)C4)CC3)no2)C(C)C)cc1 ; Cc1ncsc1-c1ccc([C@H](C)NC(=O)[C@@H]2C[C@@H](O)CN2C(=O)[C@@H](c2cc(N3CCC(CN4CC[C@@H](OC(=O)N5CCN(c6cc(-c7ccccc7O)nnc6N)CC5)[C@H](F)C4)CC3)no2)C(C)C)cc1] 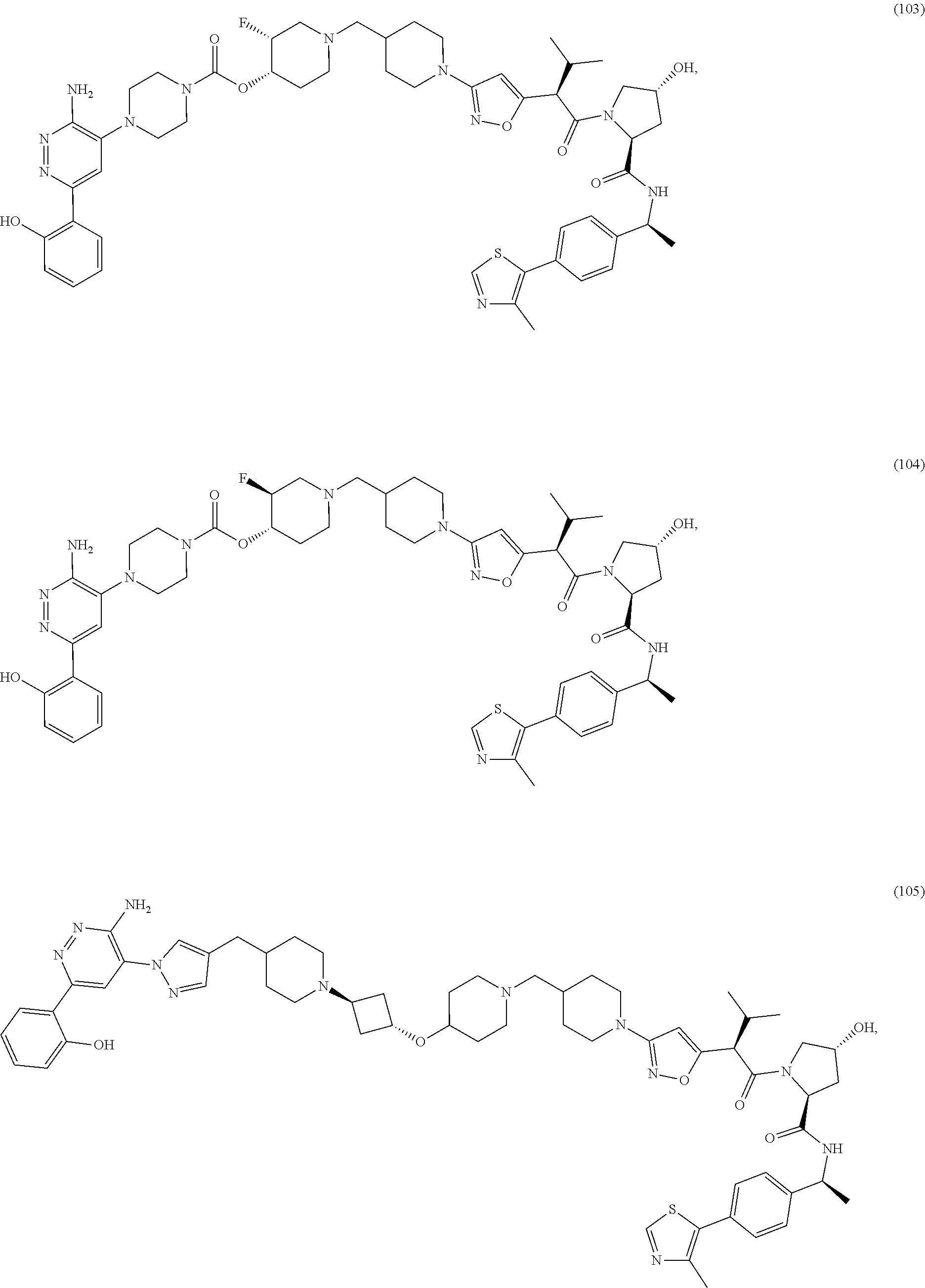 [SMILES: Cc1ncsc1-c1ccc([C@H](C)NC(=O)[C@@H]2C[C@@H](O)CN2C(=O)[C@@H](c2cc(N3CCC(CN4CC[C@H](OC(=O)N5CCN(c6cc(-c7ccccc7O)nnc6N)CC5)[C@H](F)C4)CC3)no2)C(C)C)cc1 ; Cc1ncsc1-c1ccc([C@H](C)NC(=O)[C@@H]2C[C@@H](O)CN2C(=O)[C@@H](c2cc(N3CCC(CN4CC[C@H](OC(=O)N5CCN(c6cc(-c7ccccc7O)nnc6N)CC5)[C@@H](F)C4)CC3)no2)C(C)C)cc1 ; Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCC(Cc7cnn(-c8cc(-c9ccccc9O)nnc8N)c7)CC6)C5)CC4)CC3)no2)C(C)C)cc1] 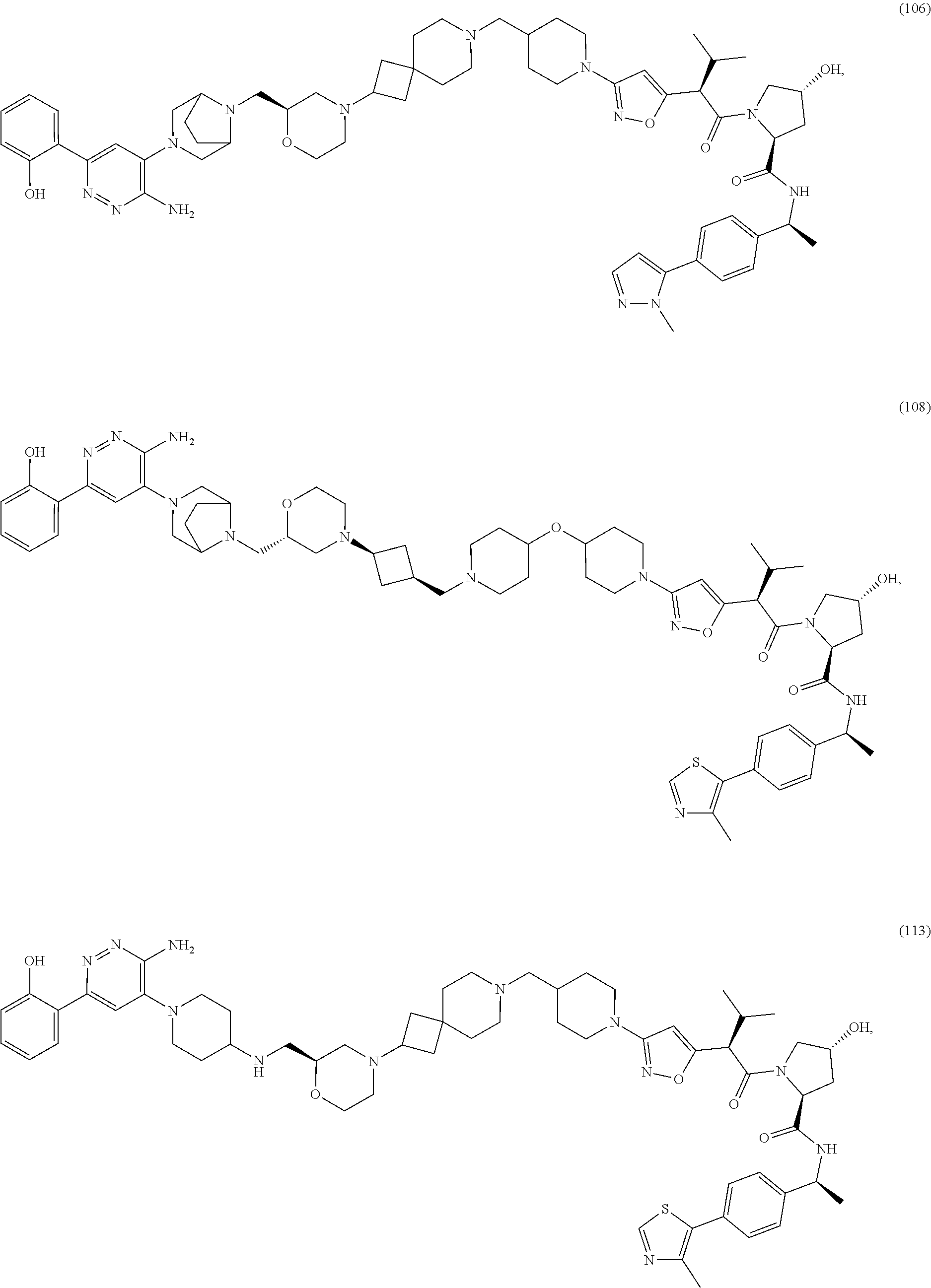 [SMILES: CC(C)[C@@H](C(=O)N1C[C@H](O)C[C@H]1C(=O)N[C@@H](C)c1ccc(-c2ccnn2C)cc1)c1cc(N2CCC(CN3CCC4(CC3)CC(N3CCO[C@@H](CN5C6CCC5CN(c5cc(-c7ccccc7O)nnc5N)C6)C3)C4)CC2)no1 ; Cc1ncsc1-c1ccc([C@H](C)NC(=O)[C@@H]2C[C@@H](O)CN2C(=O)[C@@H](c2cc(N3CCC(OC4CCN(C[C@H]5C[C@@H](N6CCO[C@@H](CN7C8CCC7CN(c7cc(-c9ccccc9O)nnc7N)C8)C6)C5)CC4)CC3)no2)C(C)C)cc1 ; Cc1ncsc1-c1ccc([C@H](C)NC(=O)[C@@H]2C[C@@H](O)CN2C(=O)[C@@H](c2cc(N3CCC(CN4CCC5(CC4)CC(N4CCO[C@@H](CNC6CCN(c7cc(-c8ccccc8O)nnc7N)CC6)C4)C5)CC3)no2)C(C)C)cc1] 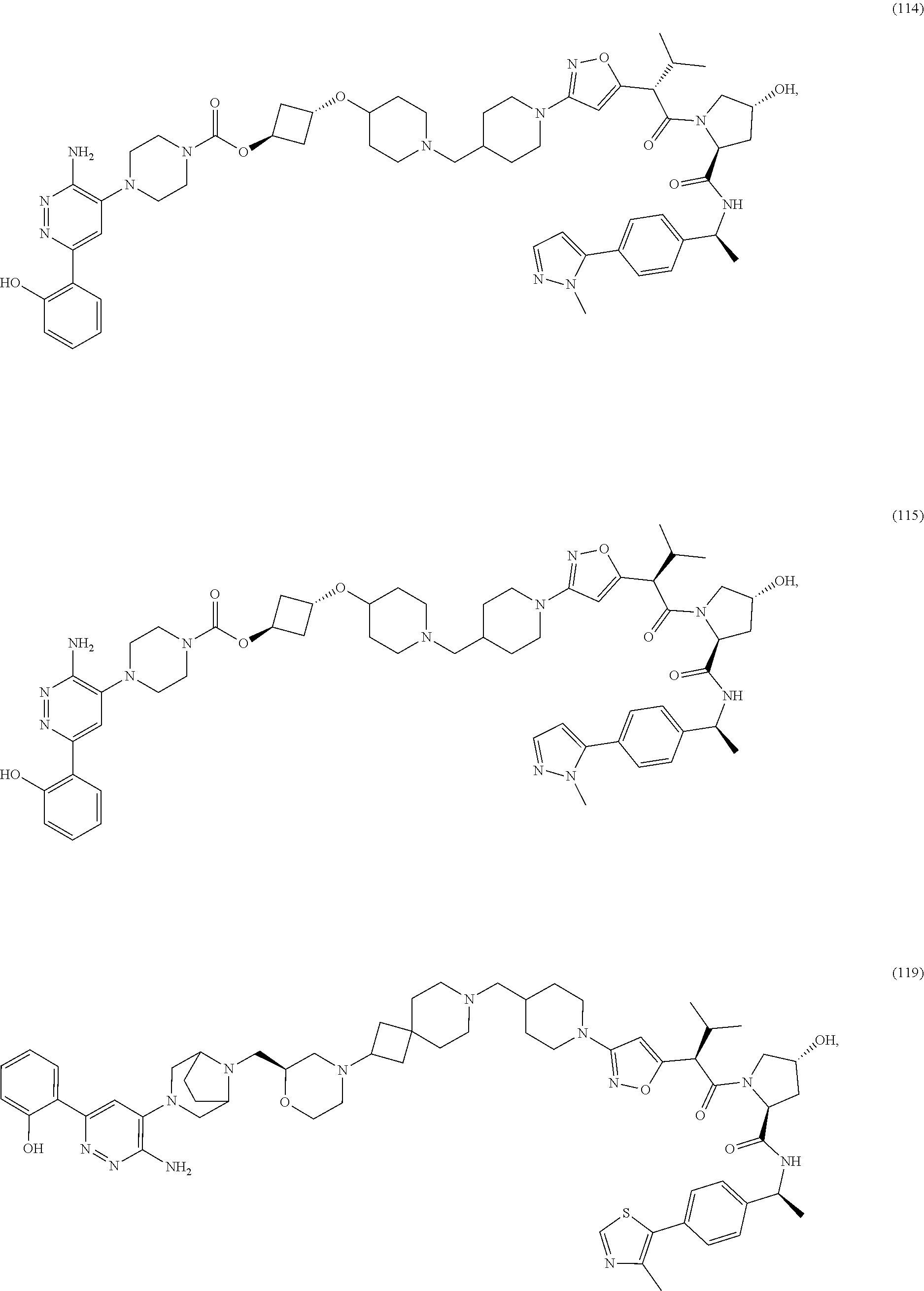 [SMILES: CC(C)[C@H](C(=O)N1C[C@H](O)C[C@H]1C(=O)N[C@@H](C)c1ccc(-c2ccnn2C)cc1)c1cc(N2CCC(CN3CCC(O[C@H]4C[C@H](OC(=O)N5CCN(c6cc(-c7ccccc7O)nnc6N)CC5)C4)CC3)CC2)no1 ; CC(C)[C@@H](C(=O)N1C[C@H](O)C[C@H]1C(=O)N[C@@H](C)c1ccc(-c2ccnn2C)cc1)c1cc(N2CCC(CN3CCC(O[C@H]4C[C@H](OC(=O)N5CCN(c6cc(-c7ccccc7O)nnc6N)CC5)C4)CC3)CC2)no1 ; Cc1ncsc1-c1ccc([C@H](C)NC(=O)[C@@H]2C[C@@H](O)CN2C(=O)[C@@H](c2cc(N3CCC(CN4CCC5(CC4)CC(N4CCO[C@@H](CN6C7CCC6CN(c6cc(-c8ccccc8O)nnc6N)C7)C4)C5)CC3)no2)C(C)C)cc1] 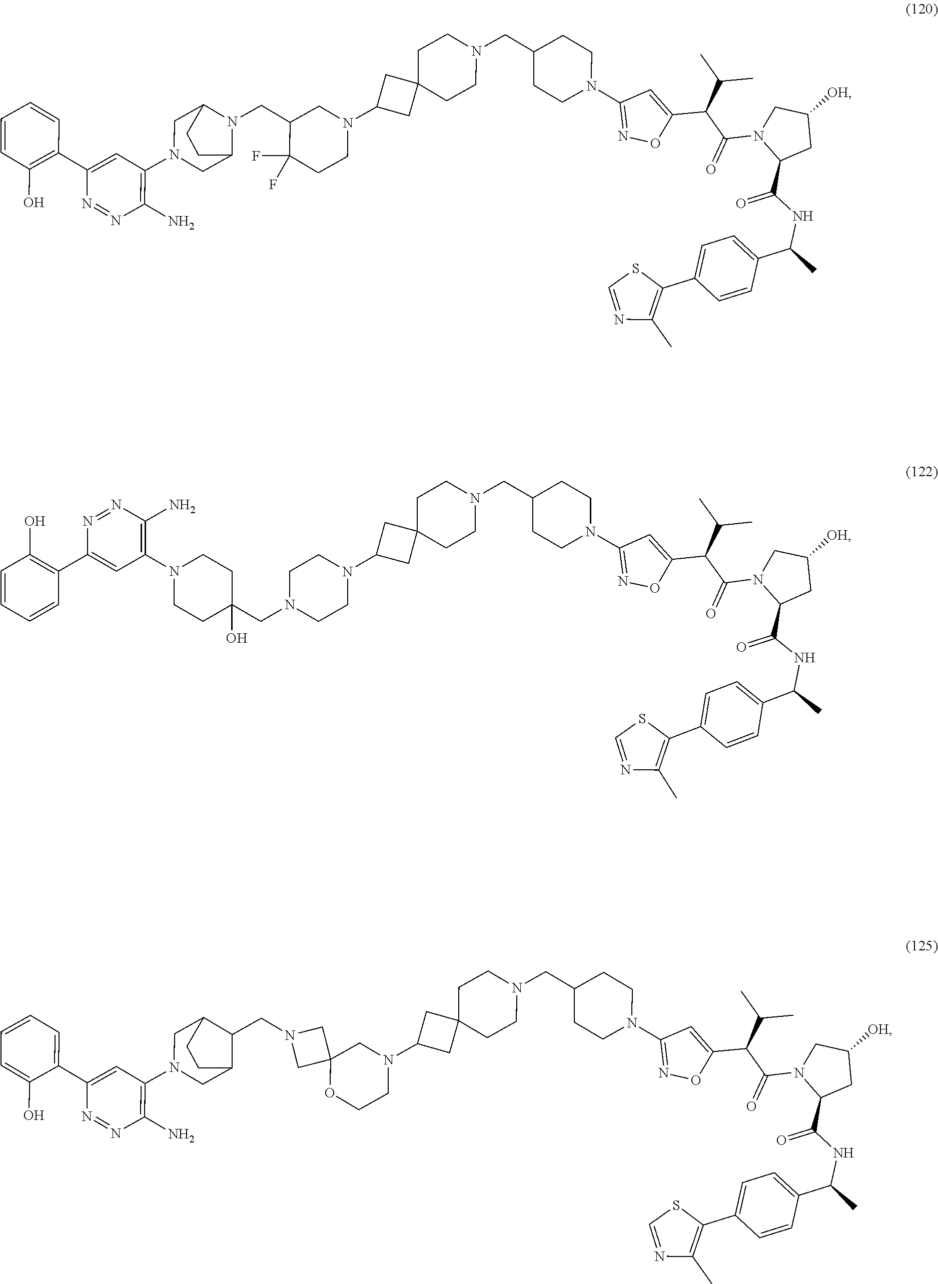 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(N4CCC(F)(F)C(CN6C7CCC6CN(c6cc(-c8ccccc8O)nnc6N)C7)C4)C5)CC3)no2)C(C)C)cc1 ; Cc1ncsc1-c1ccc([C@H](C)NC(=O)[C@@H]2C[C@@H](O)CN2C(=O)[C@@H](c2cc(N3CCC(CN4CCC5(CC4)CC(N4CCN(CC6(O)CCN(c7cc(-c8ccccc8O)nnc7N)CC6)CC4)C5)CC3)no2)C(C)C)cc1 ; Cc1ncsc1-c1ccc([C@H](C)NC(=O)[C@@H]2C[C@@H](O)CN2C(=O)[C@@H](c2cc(N3CCC(CN4CCC5(CC4)CC(N4CCOC6(CN(CC7C8CCC7CN(c7cc(-c9ccccc9O)nnc7N)C8)C6)C4)C5)CC3)no2)C(C)C)cc1] 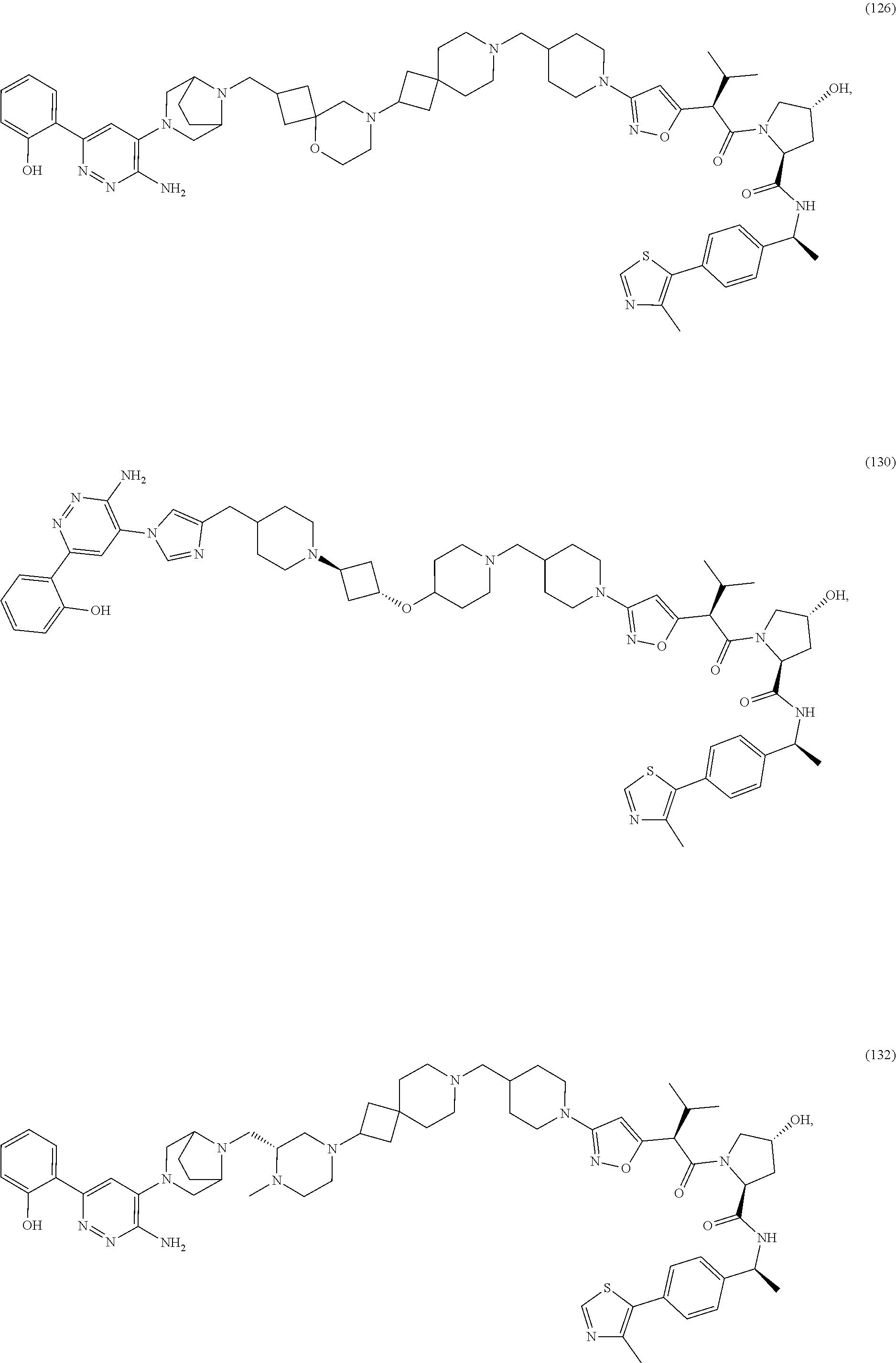 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(N4CCOC6(CC(CN7C8CCC7CN(c7cc(-c9ccccc9O)nnc7N)C8)C6)C4)C5)CC3)no2)C(C)C)cc1 ; Cc1ncsc1-c1ccc([C@H](C)NC(=O)[C@@H]2C[C@@H](O)CN2C(=O)[C@@H](c2cc(N3CCC(CN4CCC(O[C@H]5C[C@H](N6CCC(Cc7cn(-c8cc(-c9ccccc9O)nnc8N)cn7)CC6)C5)CC4)CC3)no2)C(C)C)cc1 ; Cc1ncsc1-c1ccc([C@H](C)NC(=O)[C@@H]2C[C@@H](O)CN2C(=O)[C@@H](c2cc(N3CCC(CN4CCC5(CC4)CC(N4CCN(C)[C@H](CN6C7CCC6CN(c6cc(-c8ccccc8O)nnc6N)C7)C4)C5)CC3)no2)C(C)C)cc1] 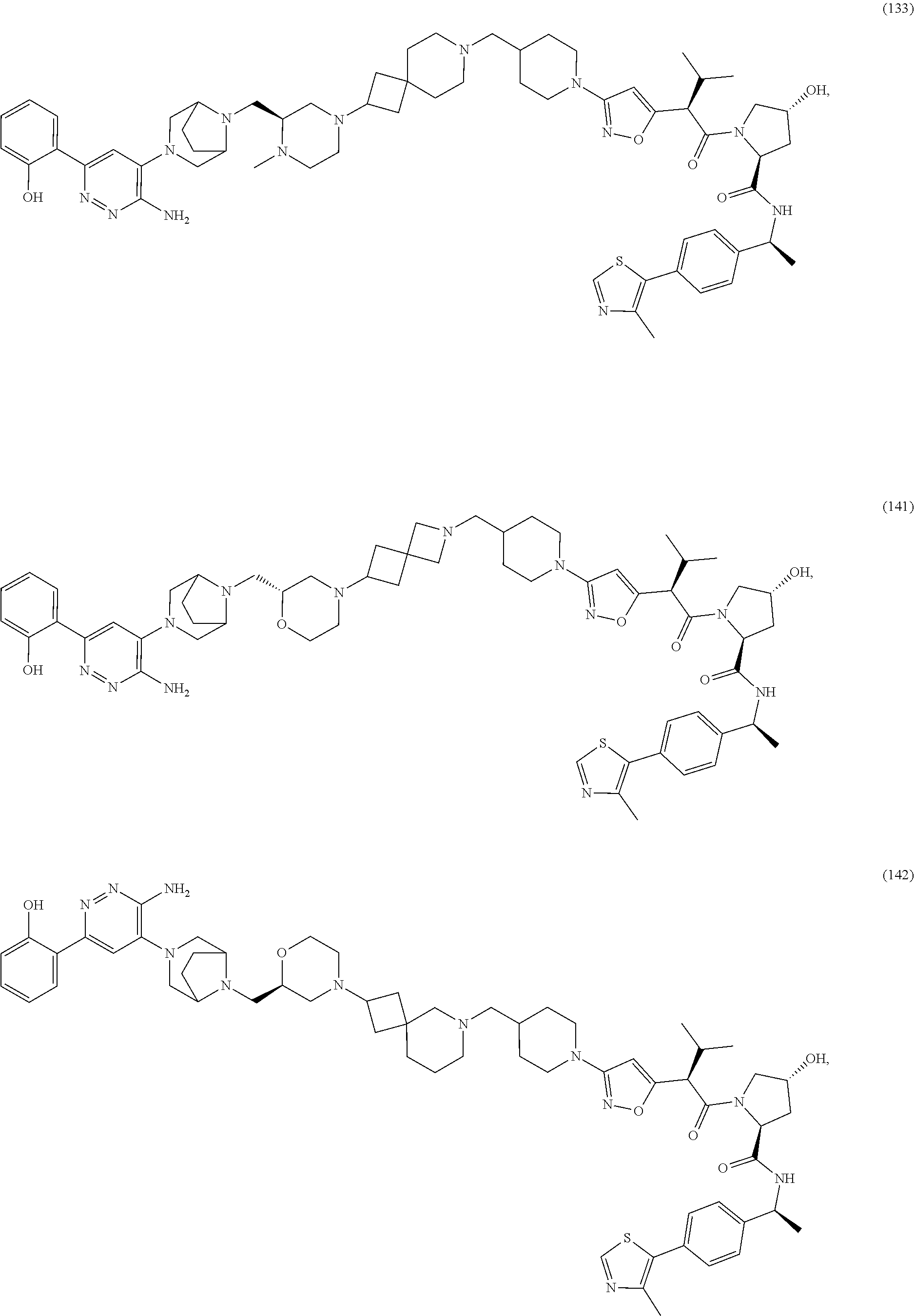 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(N4CCN(C)[C@@H](CN6C7CCC6CN(c6cc(-c8ccccc8O)nnc6N)C7)C4)C5)CC3)no2)C(C)C)cc1 ; Cc1ncsc1-c1ccc([C@H](C)NC(=O)[C@@H]2C[C@@H](O)CN2C(=O)[C@@H](c2cc(N3CCC(CN4CC5(CC(N6CCO[C@H](CN7C8CCC7CN(c7cc(-c9ccccc9O)nnc7N)C8)C6)C5)C4)CC3)no2)C(C)C)cc1 ; Cc1ncsc1-c1ccc([C@H](C)NC(=O)[C@@H]2C[C@@H](O)CN2C(=O)[C@@H](c2cc(N3CCC(CN4CCCC5(CC(N6CCO[C@H](CN7C8CCC7CN(c7cc(-c9ccccc9O)nnc7N)C8)C6)C5)C4)CC3)no2)C(C)C)cc1] 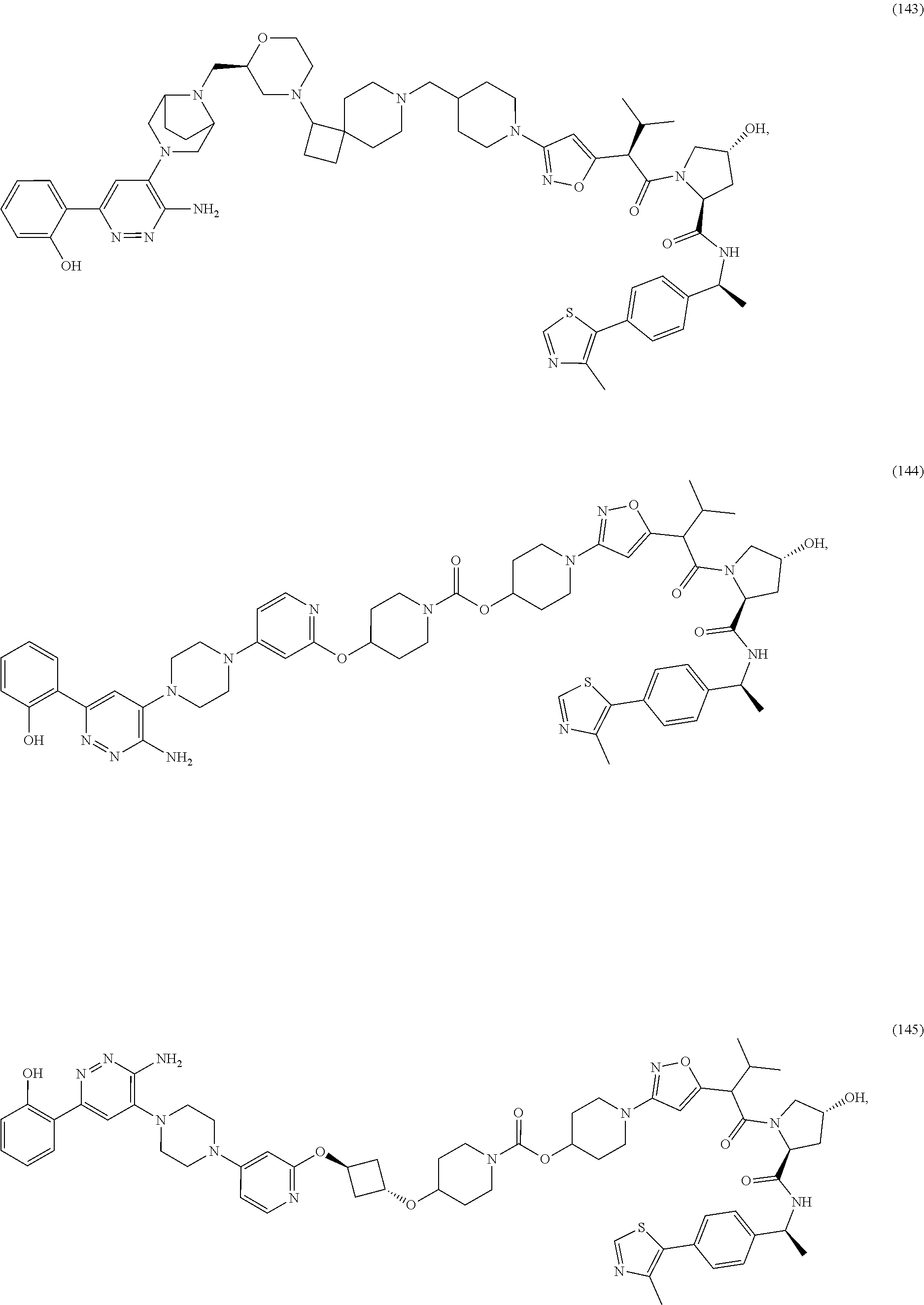 [SMILES: Cc1ncsc1-c1ccc([C@H](C)NC(=O)[C@@H]2C[C@@H](O)CN2C(=O)[C@@H](c2cc(N3CCC(CN4CCC5(CCC5N5CCO[C@H](CN6C7CCC6CN(c6cc(-c8ccccc8O)nnc6N)C7)C5)CC4)CC3)no2)C(C)C)cc1 ; Cc1ncsc1-c1ccc([C@H](C)NC(=O)[C@@H]2C[C@@H](O)CN2C(=O)C(c2cc(N3CCC(OC(=O)N4CCC(Oc5cc(N6CCN(c7cc(-c8ccccc8O)nnc7N)CC6)ccn5)CC4)CC3)no2)C(C)C)cc1 ; Cc1ncsc1-c1ccc([C@H](C)NC(=O)[C@@H]2C[C@@H](O)CN2C(=O)C(c2cc(N3CCC(OC(=O)N4CCC(O[C@H]5C[C@H](Oc6cc(N7CCN(c8cc(-c9ccccc9O)nnc8N)CC7)ccn6)C5)CC4)CC3)no2)C(C)C)cc1] 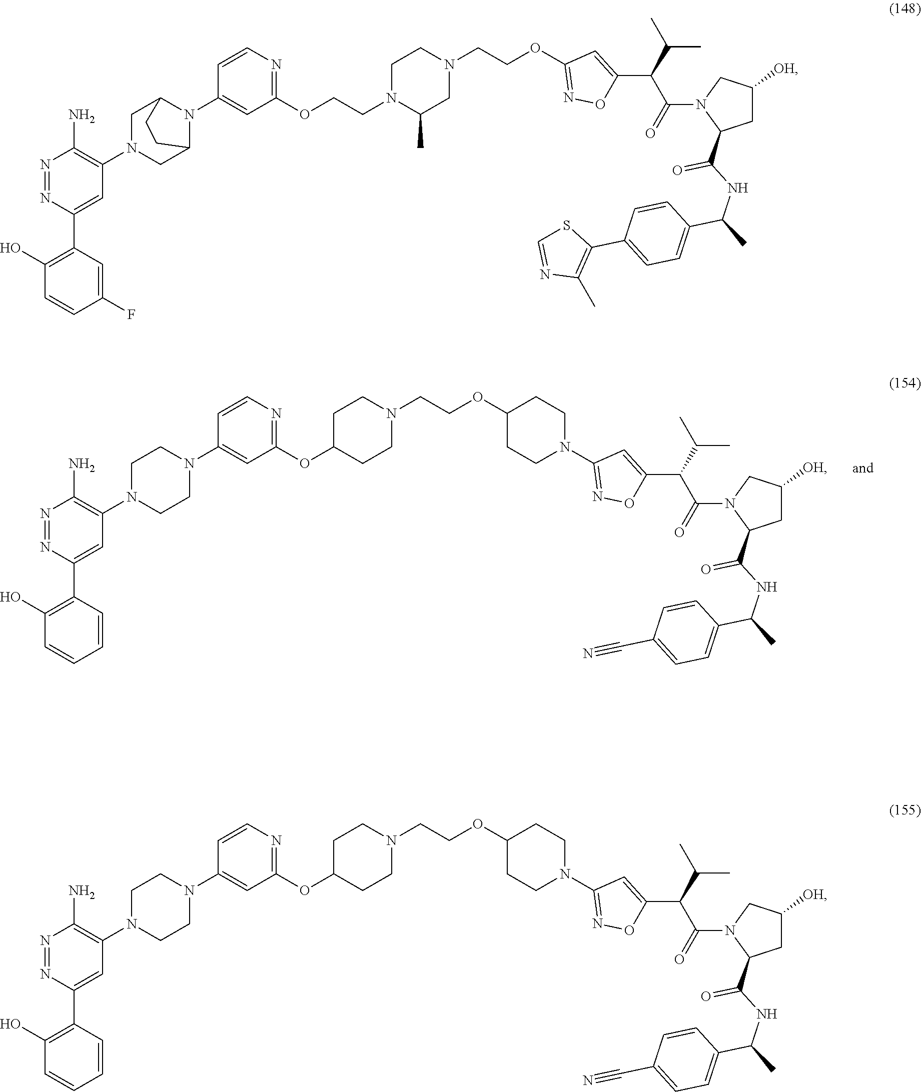 [SMILES: Cc1ncsc1-c1ccc([C@H](C)NC(=O)[C@@H]2C[C@@H](O)CN2C(=O)[C@@H](c2cc(OCCN3CCN(CCOc4cc(N5C6CCC5CN(c5cc(-c7cc(F)ccc7O)nnc5N)C6)ccn4)[C@H](C)C3)no2)C(C)C)cc1 ; CC(C)[C@H](C(=O)N1C[C@H](O)C[C@H]1C(=O)N[C@@H](C)c1ccc(C#N)cc1)c1cc(N2CCC(OCCN3CCC(Oc4cc(N5CCN(c6cc(-c7ccccc7O)nnc6N)CC5)ccn4)CC3)CC2)no1 ; CC(C)[C@@H](C(=O)N1C[C@H](O)C[C@H]1C(=O)N[C@@H](C)c1ccc(C#N)cc1)c1cc(N2CCC(OCCN3CCC(Oc4cc(N5CCN(c6cc(-c7ccccc7O)nnc6N)CC5)ccn4)CC3)CC2)no1]
or a pharmaceutically acceptable salt thereof.

28. The compounds of claim 26 selected from: 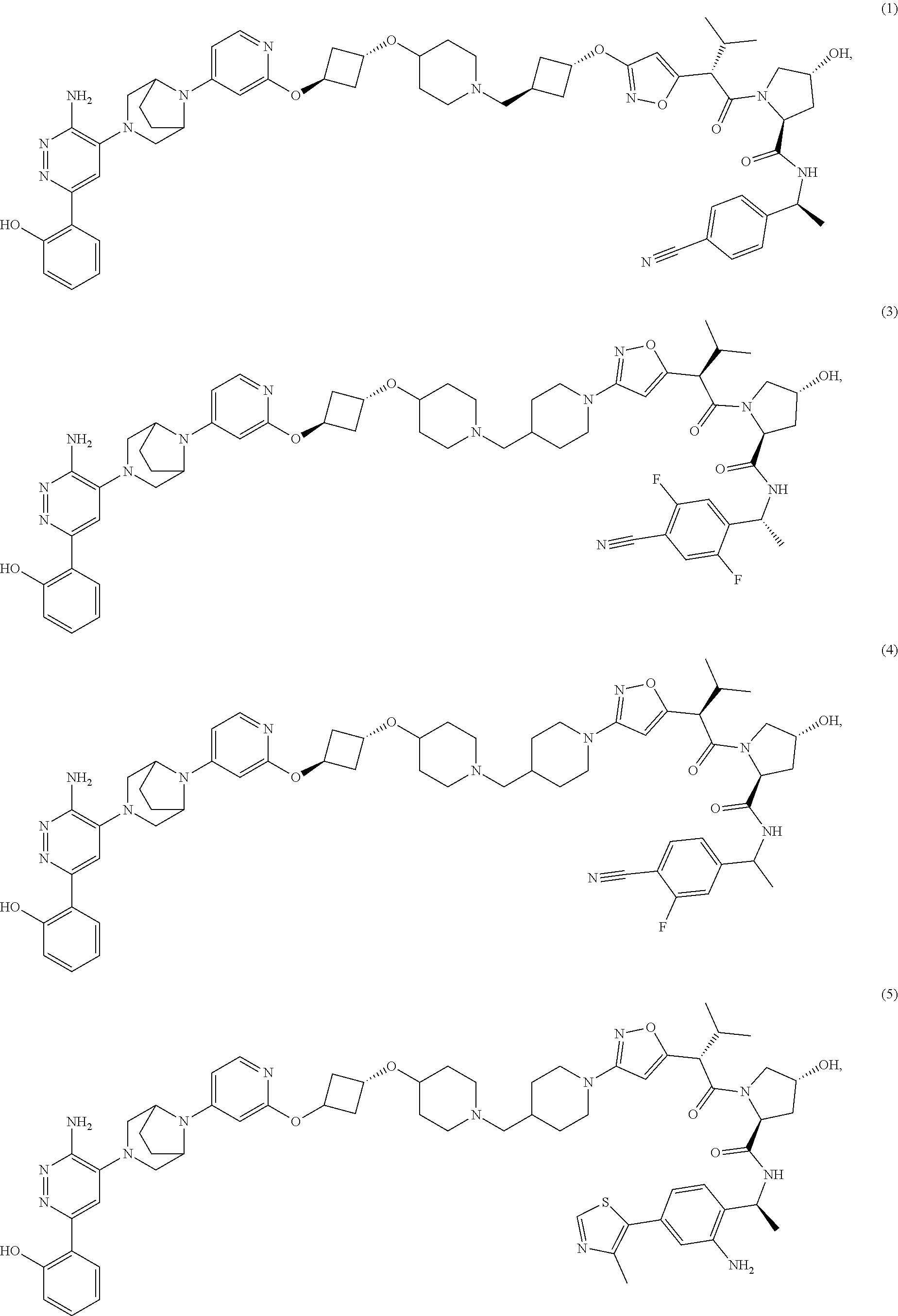 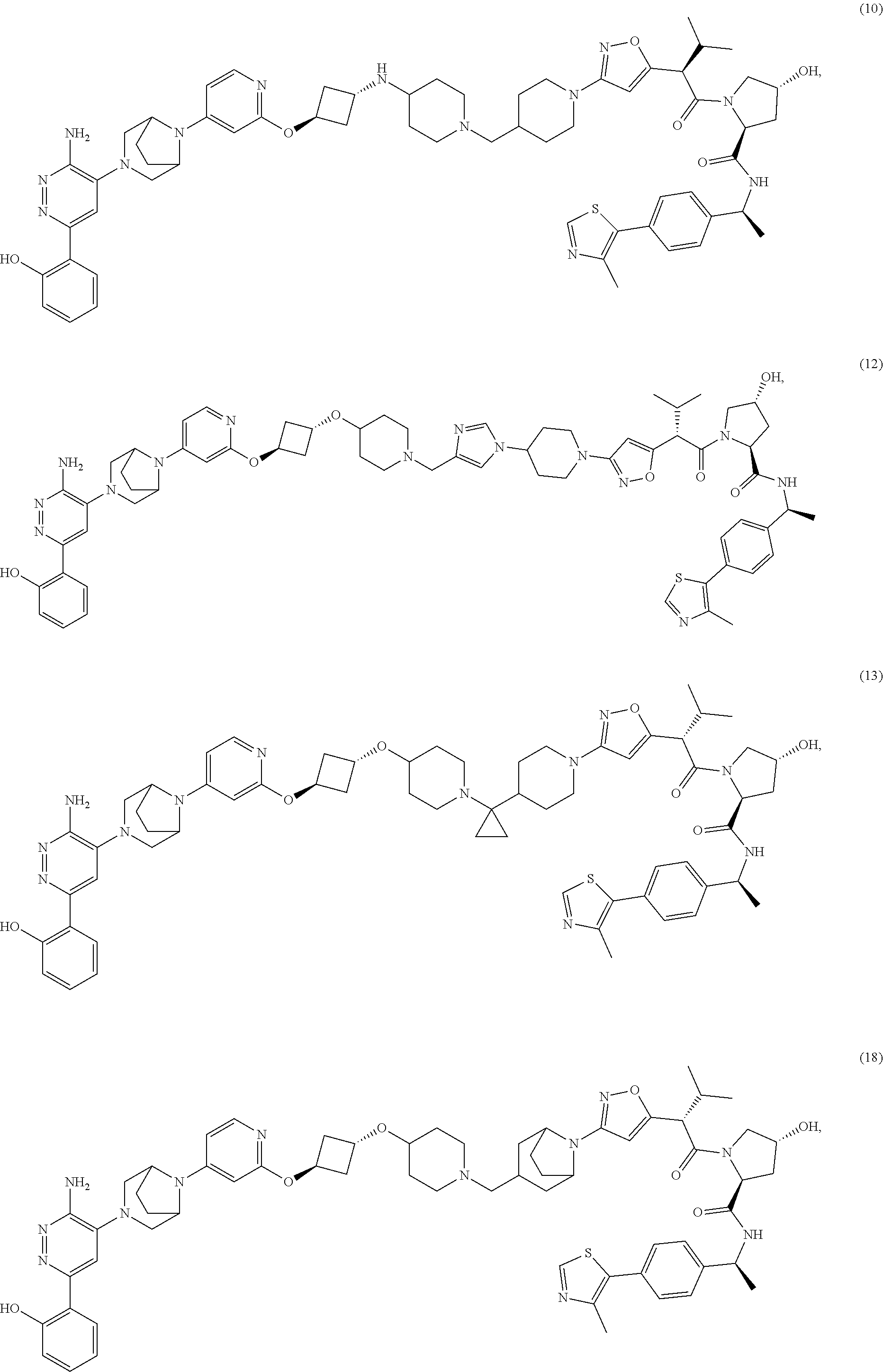 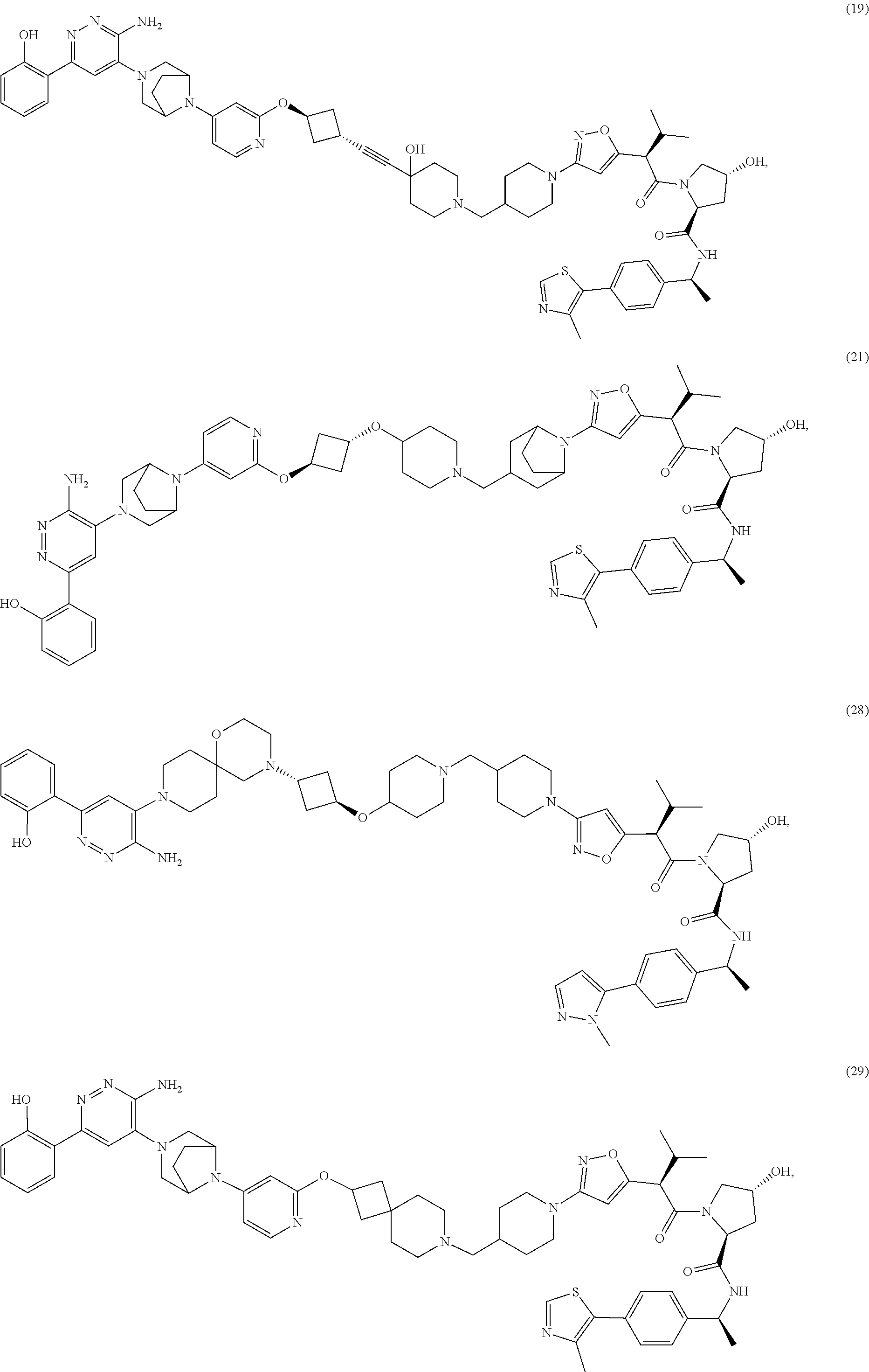 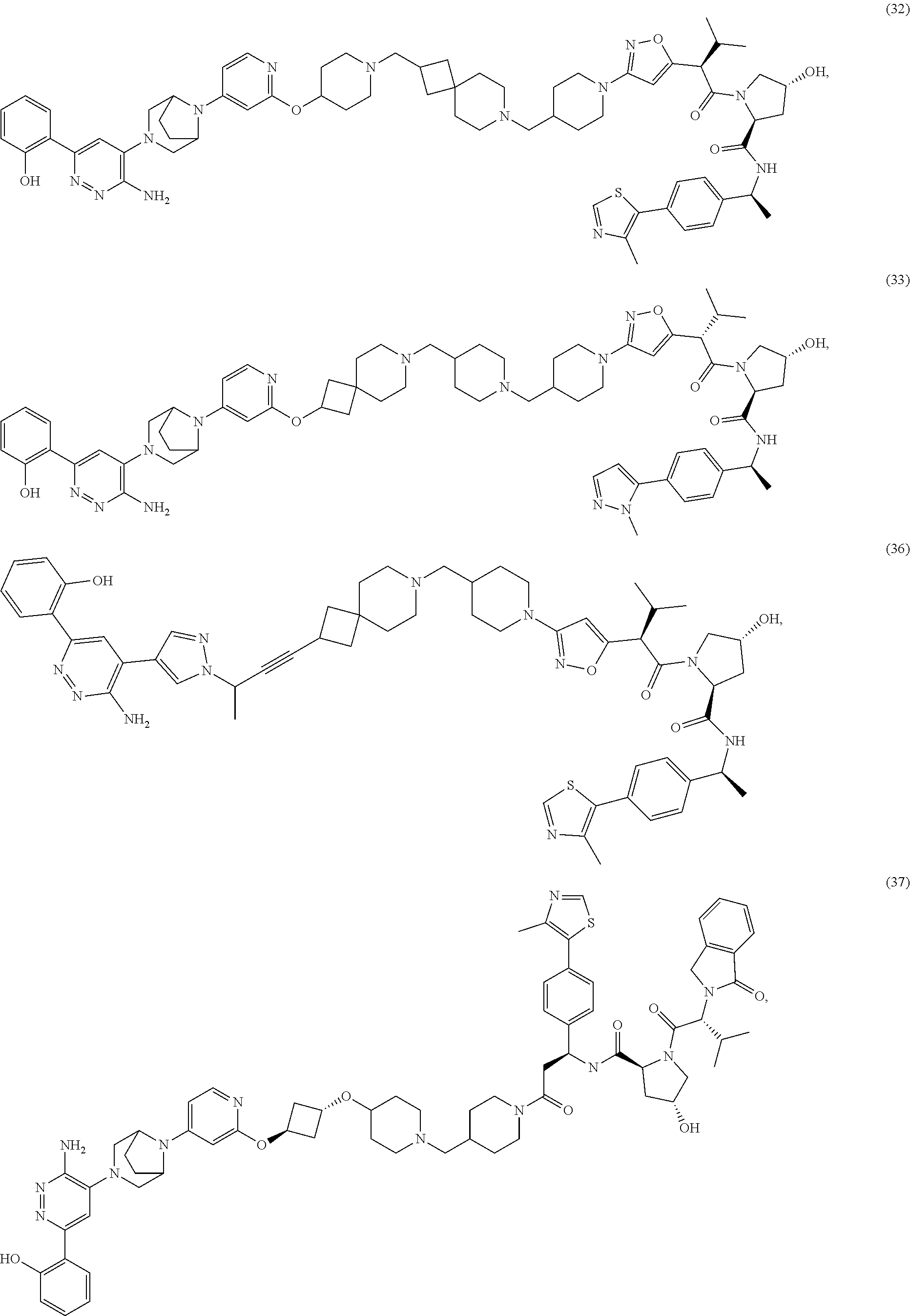 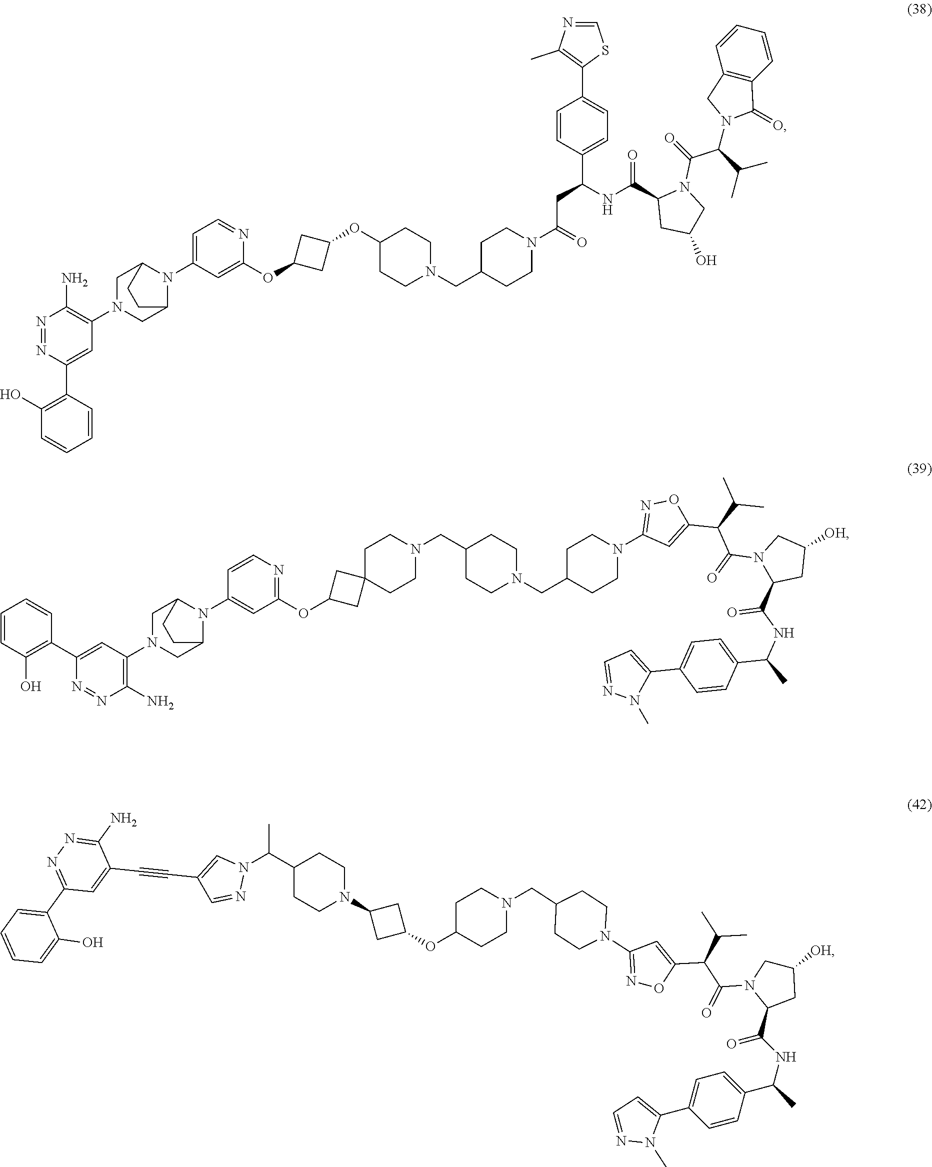 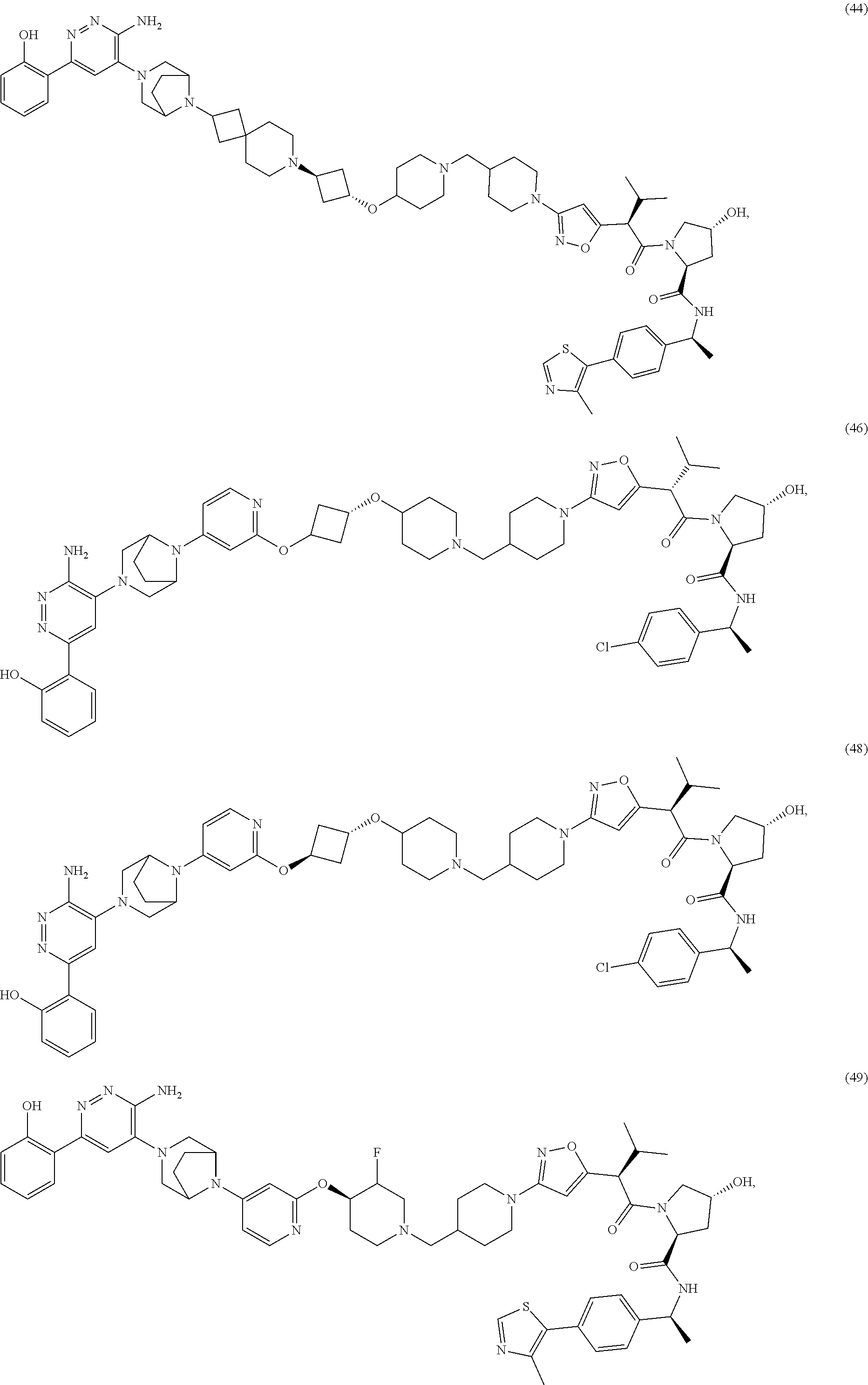 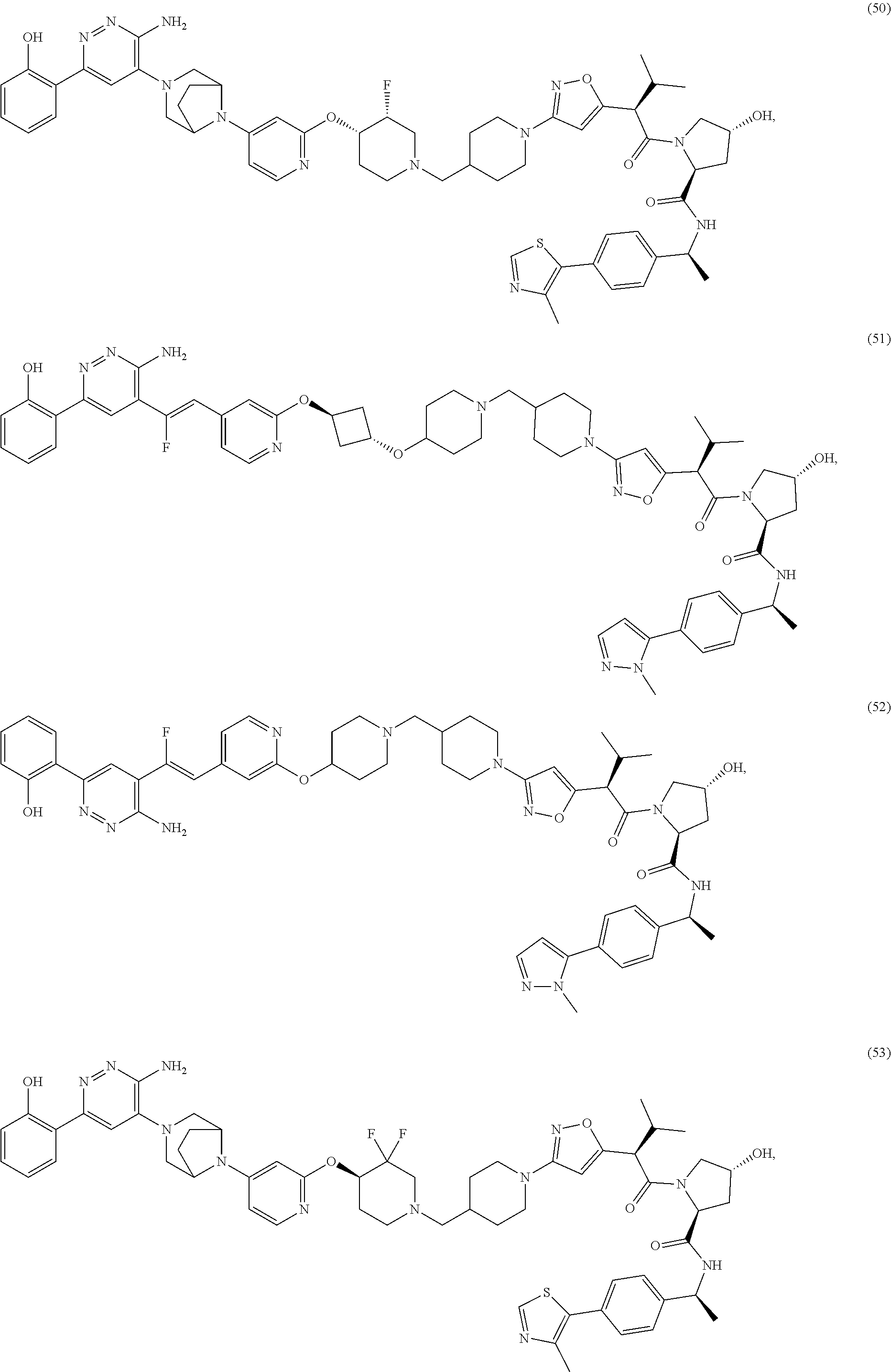 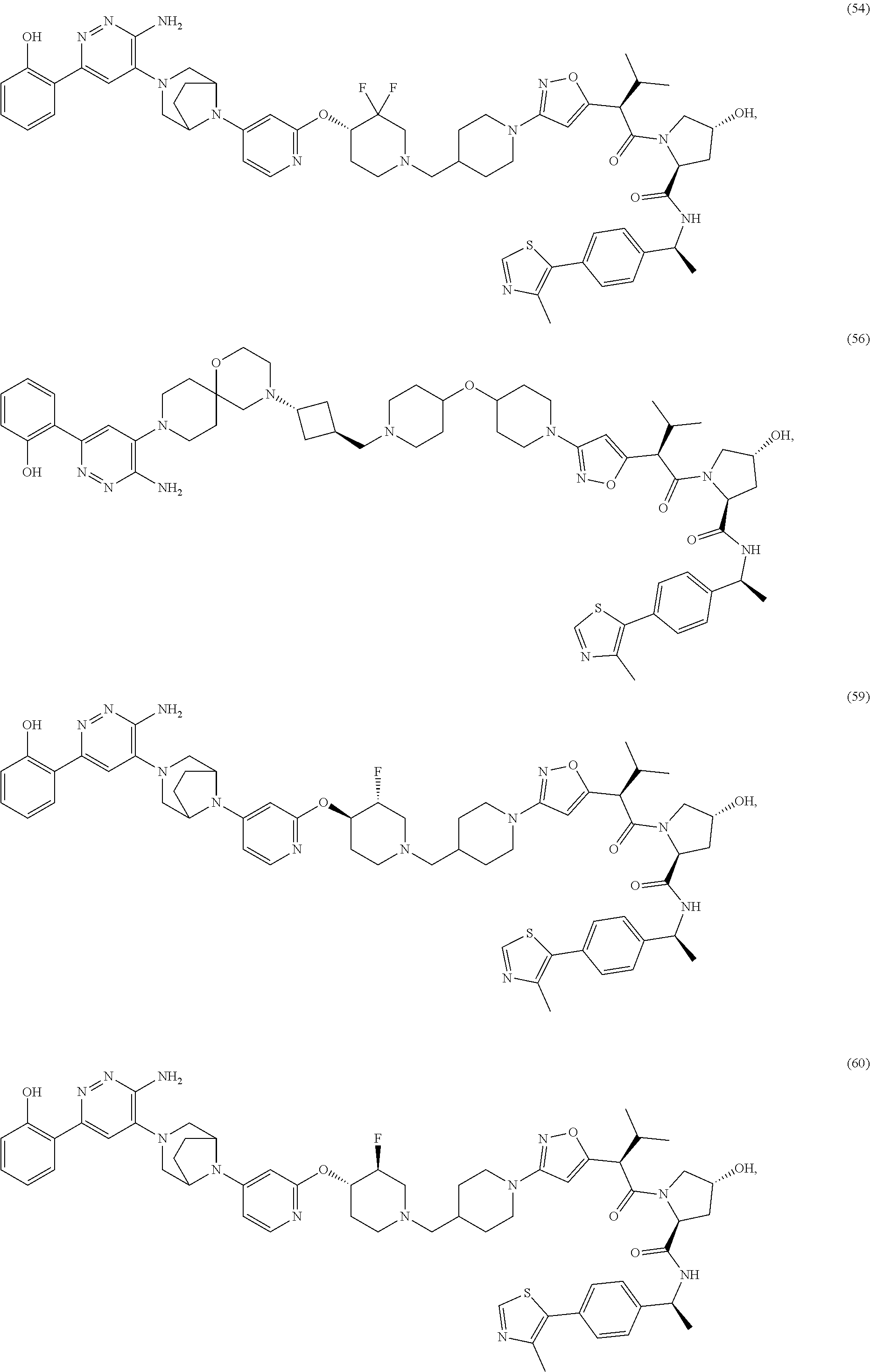 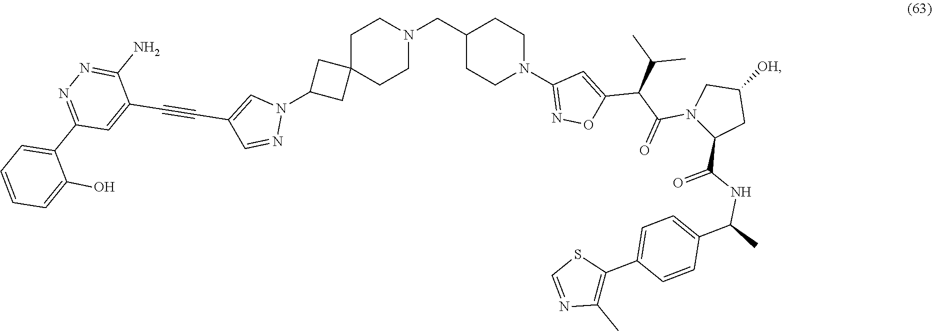 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(n4cc(C#Cc6cc(-c7ccccc7O)nnc6N)cn4)C5)CC3)no2)C(C)C)cc1] 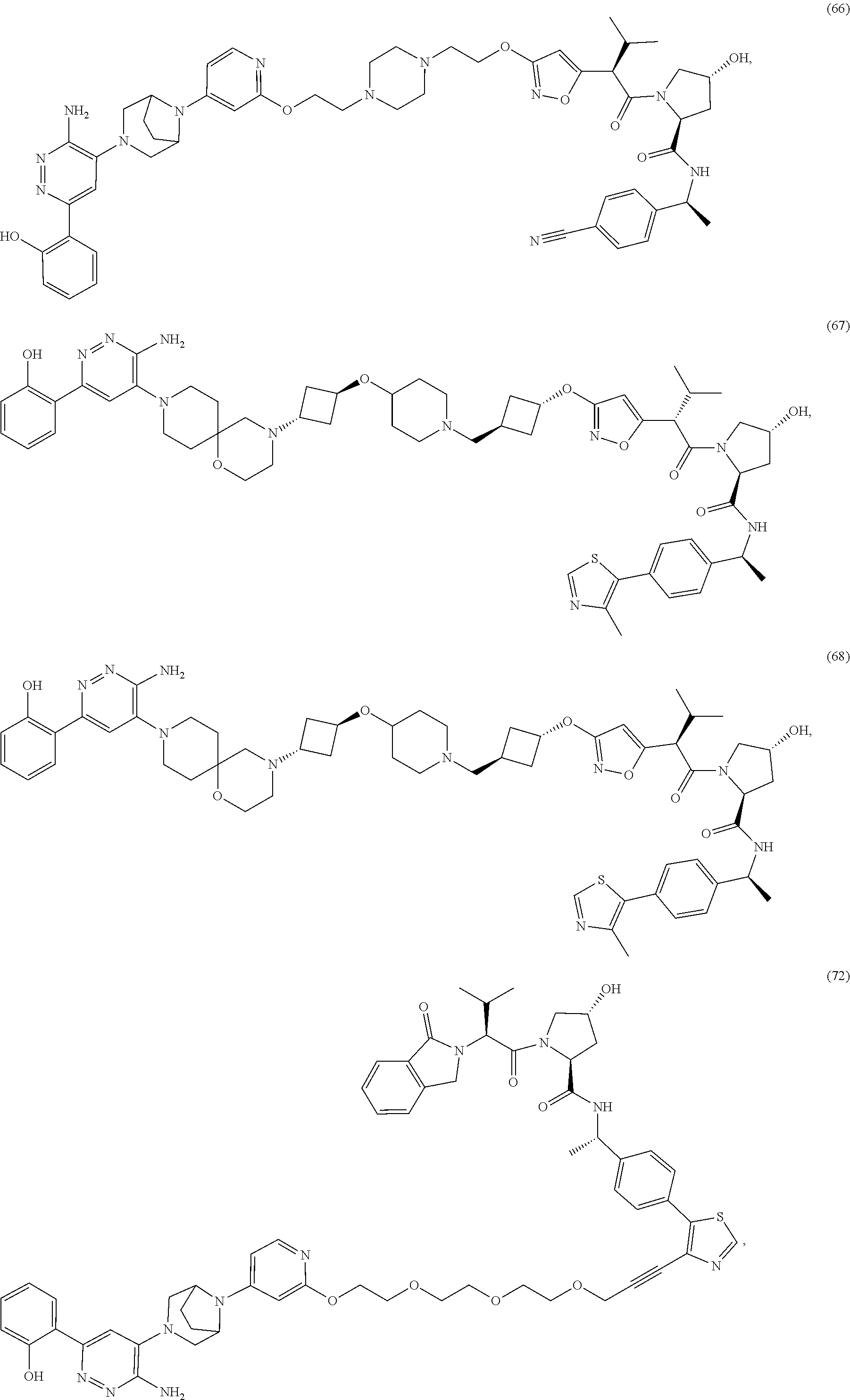 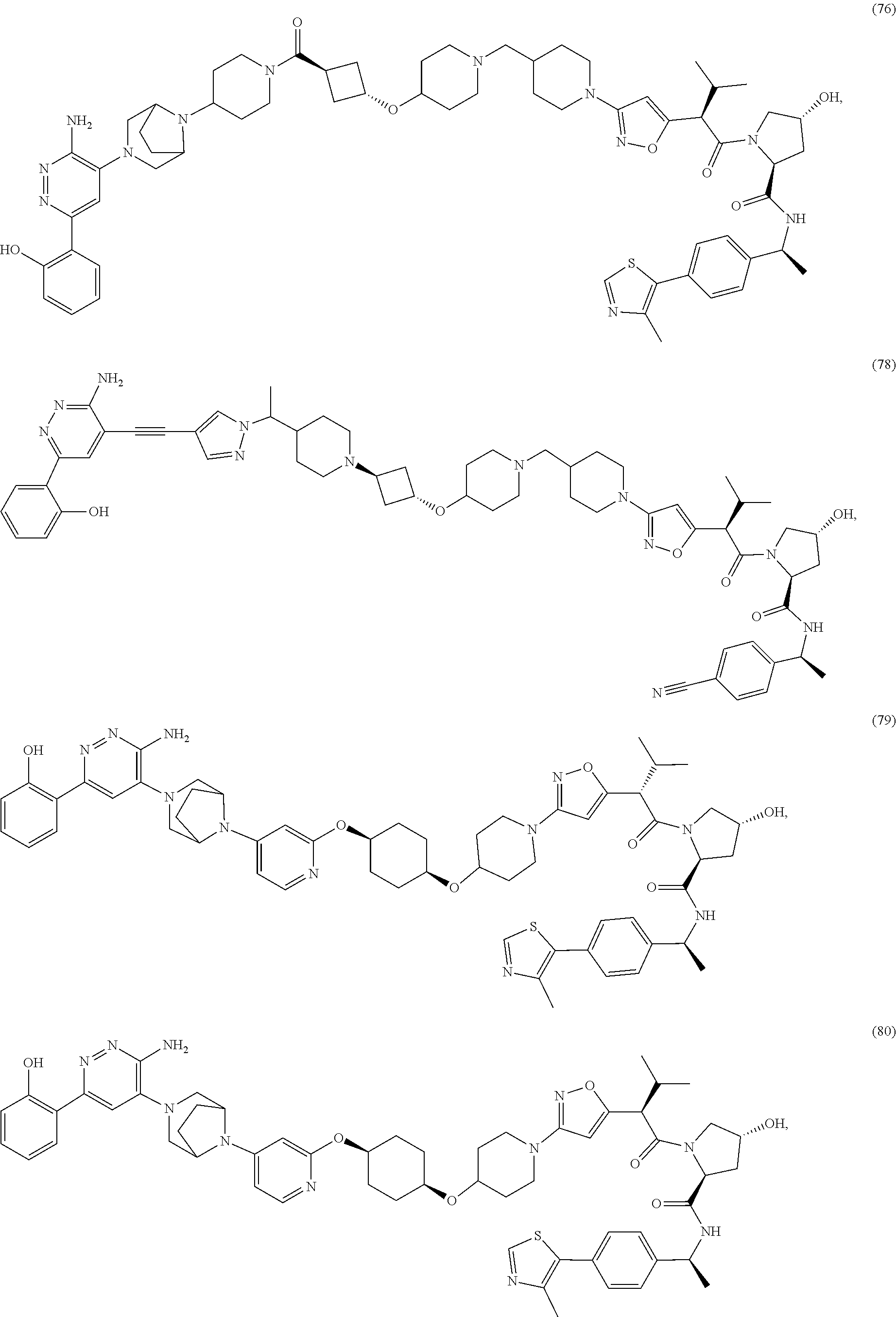 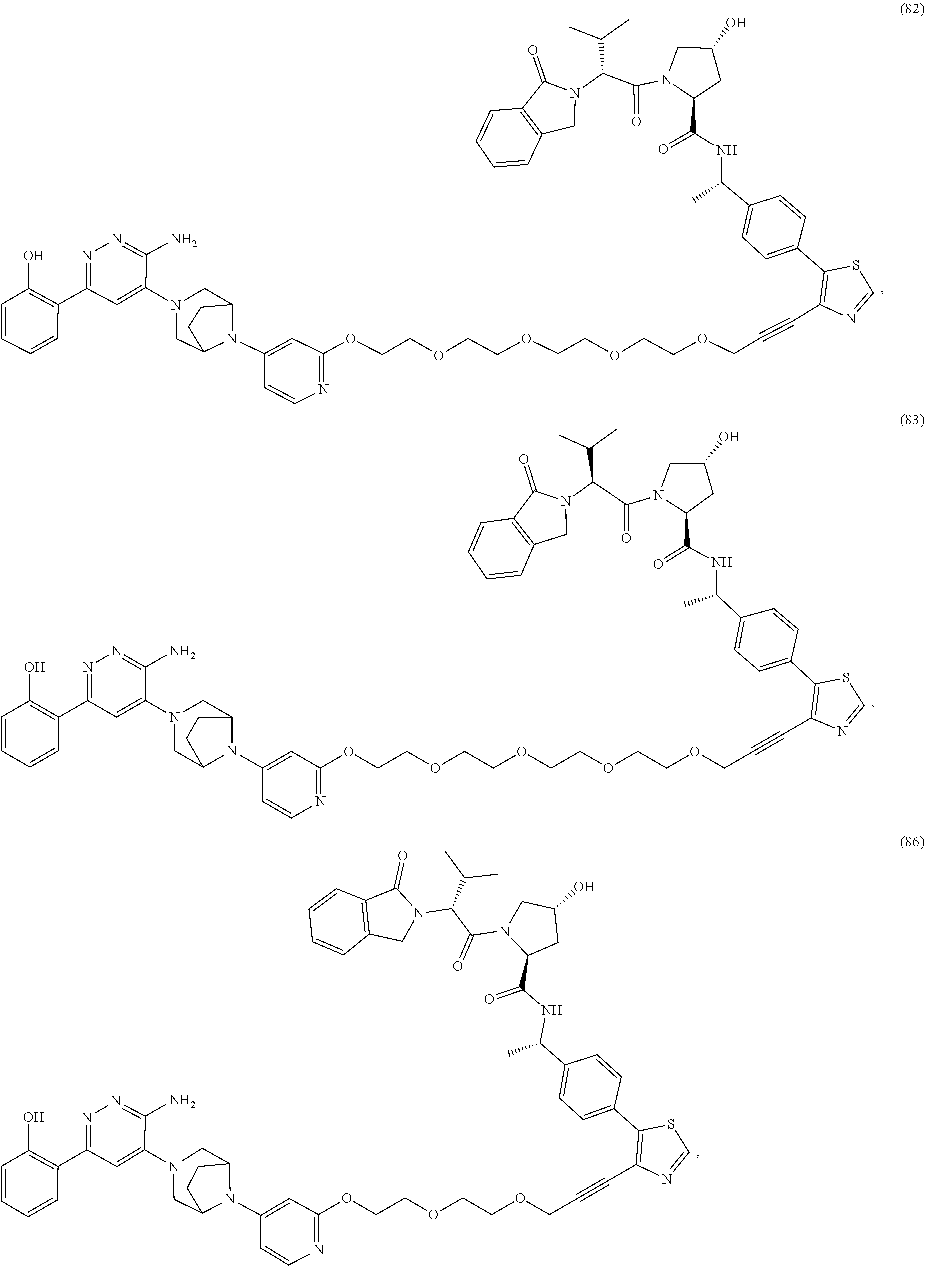 [SMILES: CC(C)[C@H](C(=O)N1C[C@H](O)C[C@H]1C(=O)N[C@@H](C)c1ccc(-c2scnc2C#CCOCCOCCOCCOCCOc2cc(N3C4CCC3CN(c3cc(-c5ccccc5O)nnc3N)C4)ccn2)cc1)N1Cc2ccccc2C1=O ; CC(C)[C@@H](C(=O)N1C[C@H](O)C[C@H]1C(=O)N[C@@H](C)c1ccc(-c2scnc2C#CCOCCOCCOCCOCCOc2cc(N3C4CCC3CN(c3cc(-c5ccccc5O)nnc3N)C4)ccn2)cc1)N1Cc2ccccc2C1=O ; CC(C)[C@H](C(=O)N1C[C@H](O)C[C@H]1C(=O)N[C@@H](C)c1ccc(-c2scnc2C#CCOCCOCCOc2cc(N3C4CCC3CN(c3cc(-c5ccccc5O)nnc3N)C4)ccn2)cc1)N1Cc2ccccc2C1=O] 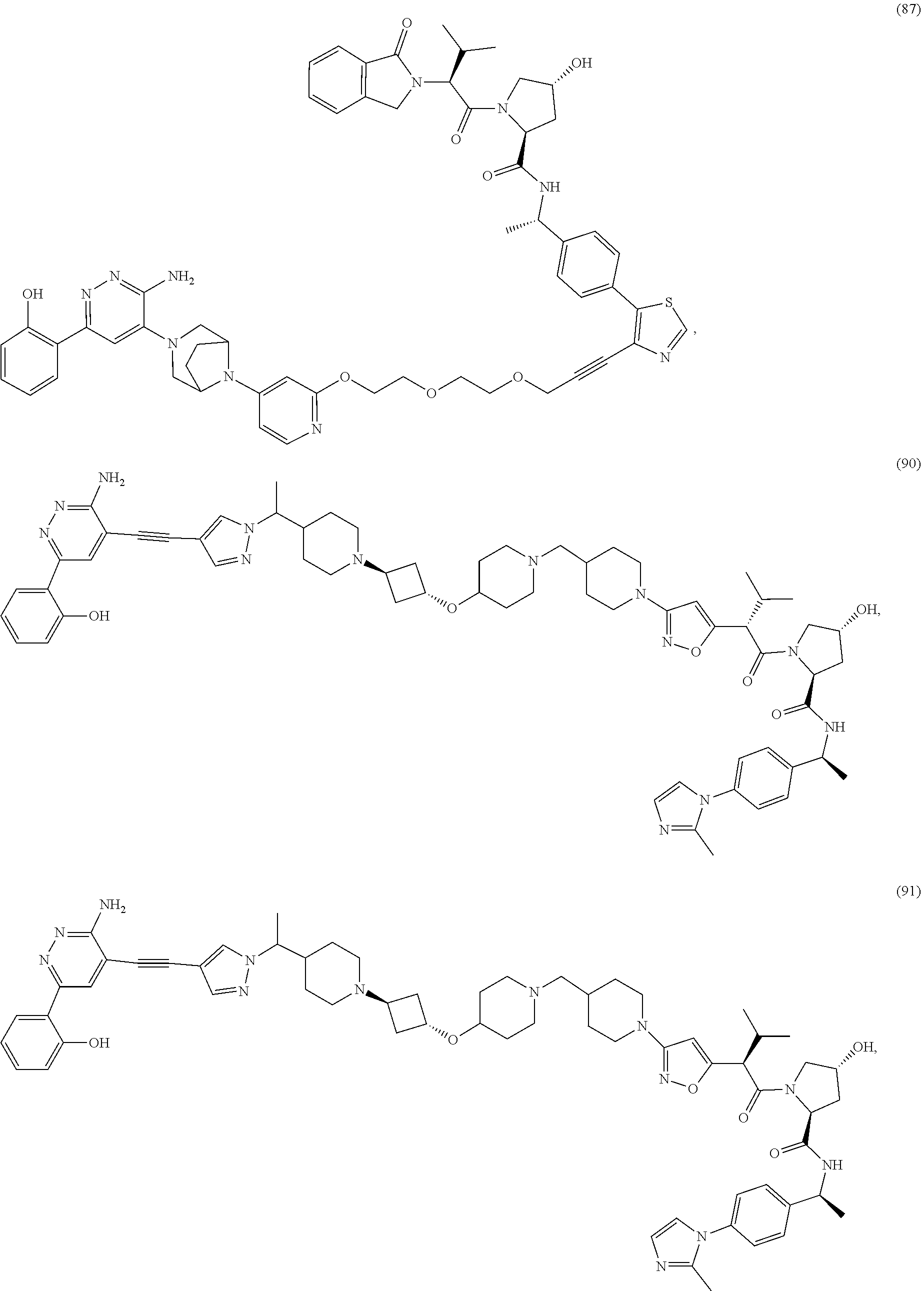 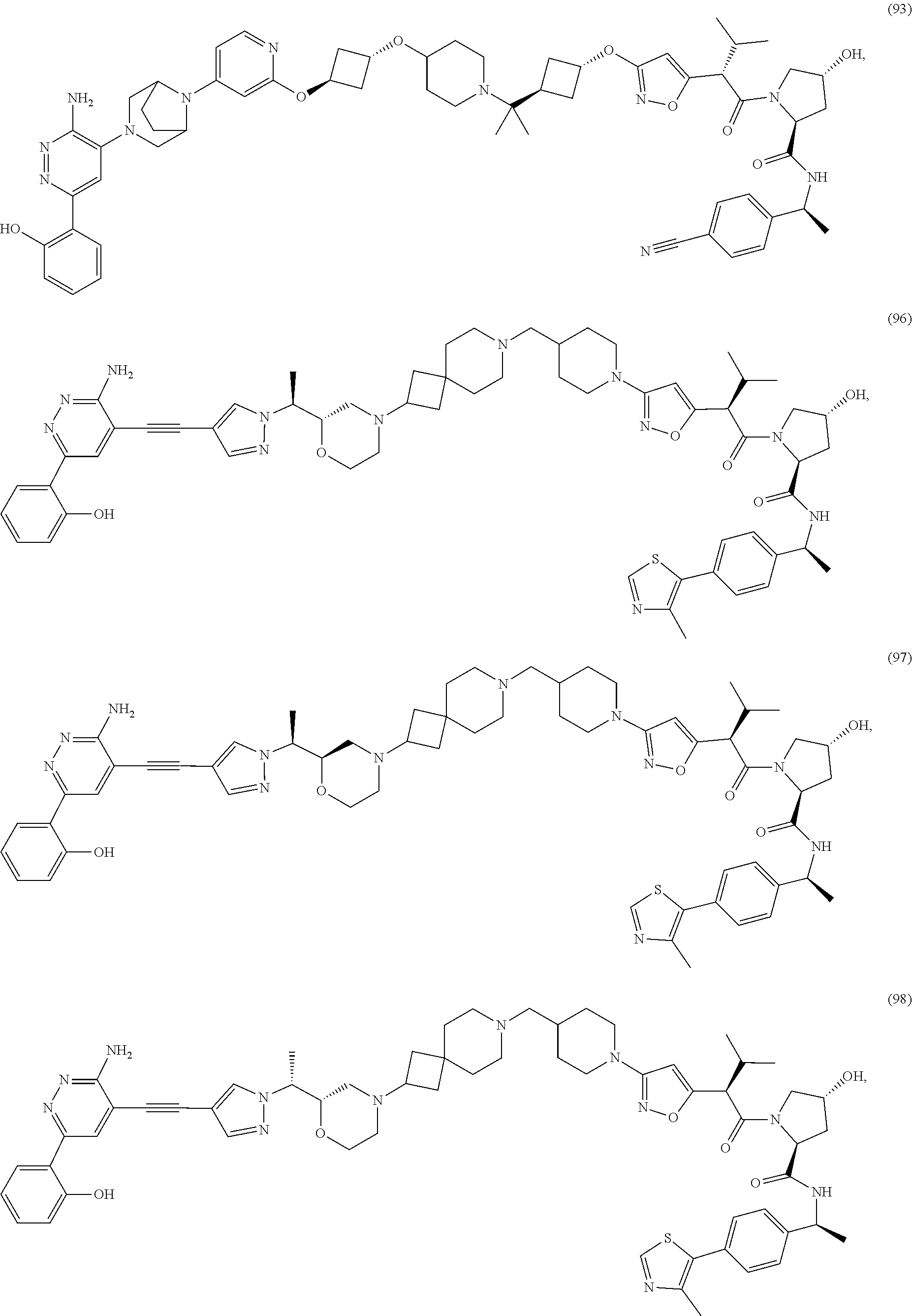 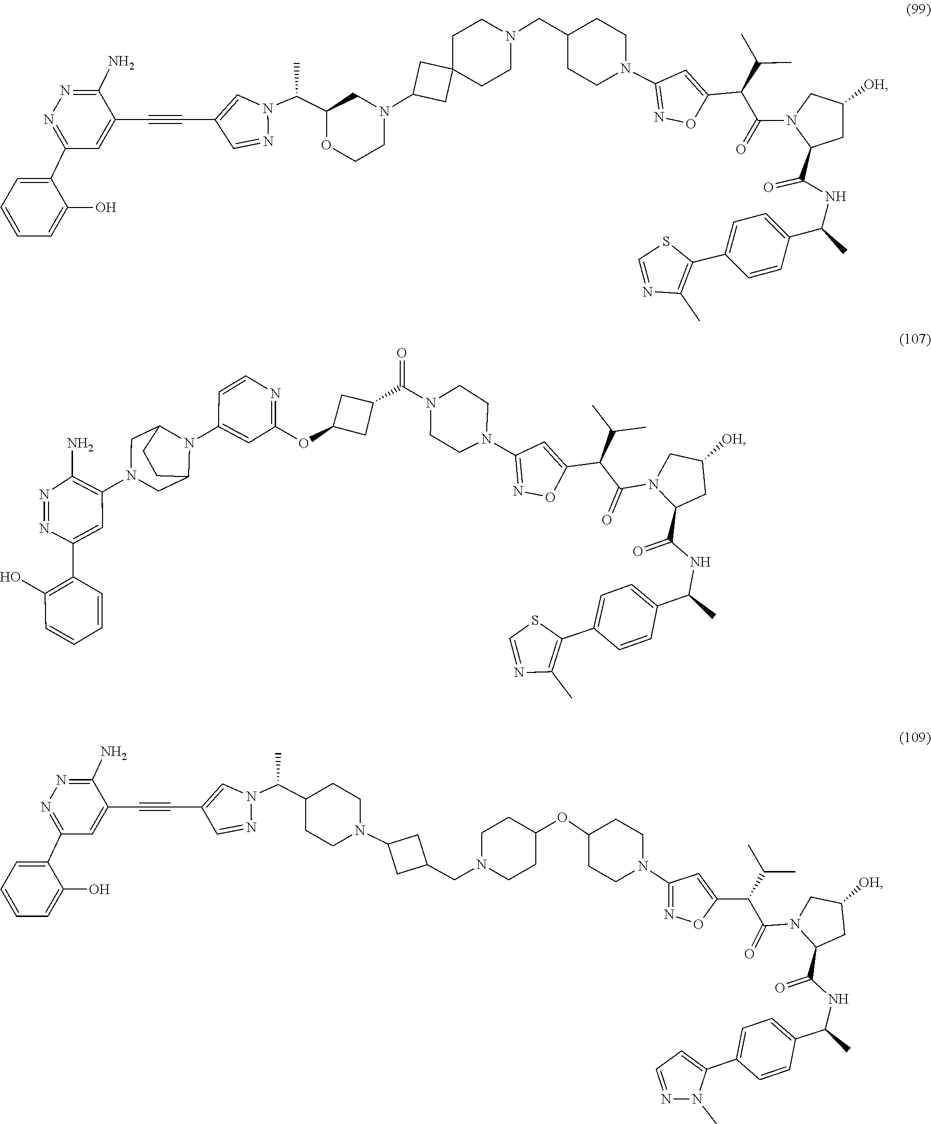 [SMILES: Cc1ncsc1-c1ccc([C@H](C)NC(=O)[C@@H]2C[C@@H](O)CN2C(=O)[C@@H](c2cc(N3CCC(CN4CCC5(CC4)CC(N4CCO[C@@H]([C@@H](C)n6cc(C#Cc7cc(-c8ccccc8O)nnc7N)cn6)C4)C5)CC3)no2)C(C)C)cc1 ; Cc1ncsc1-c1ccc([C@H](C)NC(=O)[C@@H]2C[C@@H](O)CN2C(=O)[C@@H](c2cc(N3CCN(C(=O)[C@H]4C[C@H](Oc5cc(N6C7CCC6CN(c6cc(-c8ccccc8O)nnc6N)C7)ccn5)C4)CC3)no2)C(C)C)cc1 ; CC(C)[C@H](C(=O)N1C[C@H](O)C[C@H]1C(=O)N[C@@H](C)c1ccc(-c2ccnn2C)cc1)c1cc(N2CCC(OC3CCN(CC4CC(N5CCC([C@@H](C)n6cc(C#Cc7cc(-c8ccccc8O)nnc7N)cn6)CC5)C4)CC3)CC2)no1] 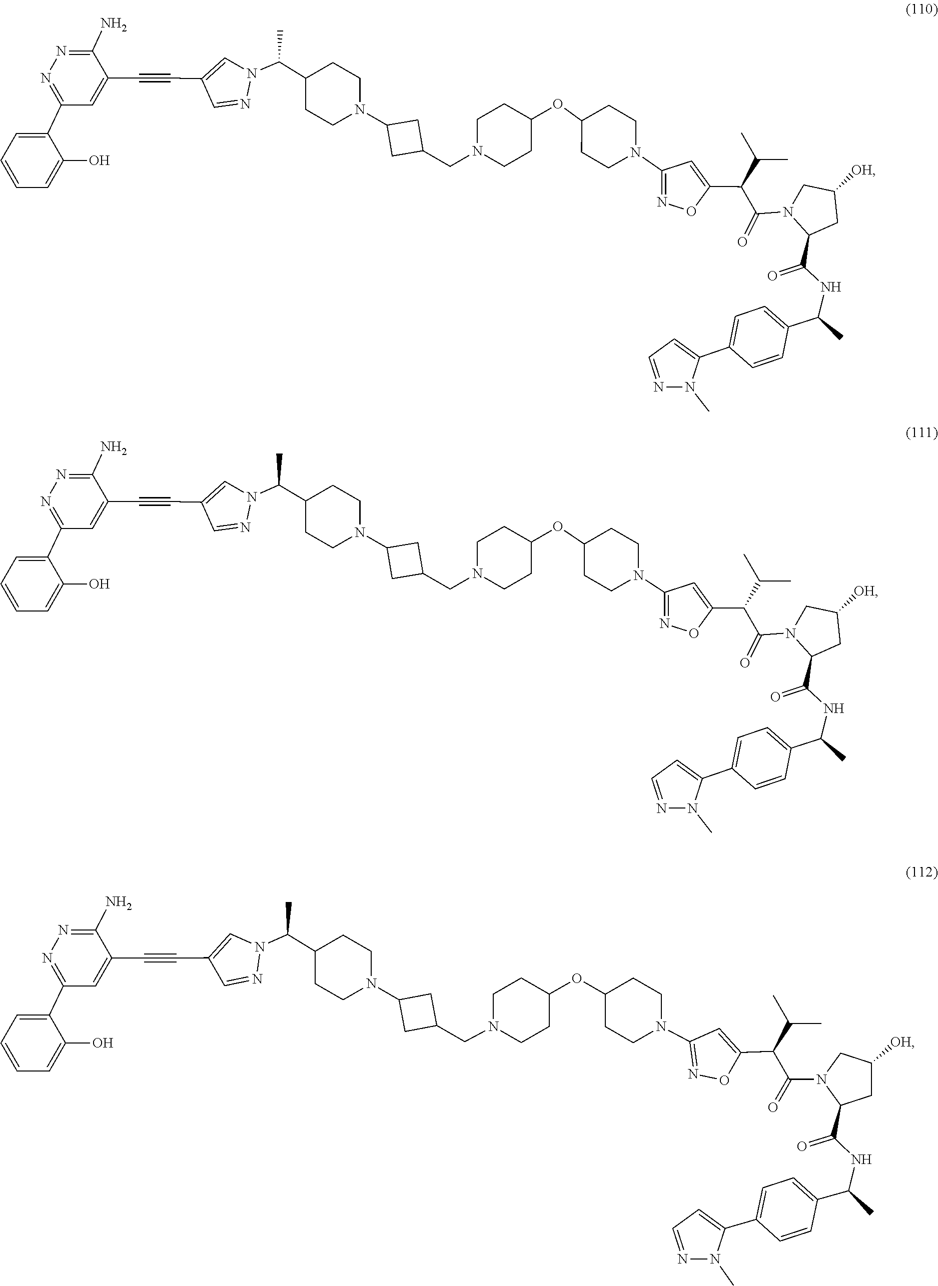 [SMILES: CC(C)[C@@H](C(=O)N1C[C@H](O)C[C@H]1C(=O)N[C@@H](C)c1ccc(-c2ccnn2C)cc1)c1cc(N2CCC(OC3CCN(CC4CC(N5CCC([C@@H](C)n6cc(C#Cc7cc(-c8ccccc8O)nnc7N)cn6)CC5)C4)CC3)CC2)no1 ; CC(C)[C@H](C(=O)N1C[C@H](O)C[C@H]1C(=O)N[C@@H](C)c1ccc(-c2ccnn2C)cc1)c1cc(N2CCC(OC3CCN(CC4CC(N5CCC([C@H](C)n6cc(C#Cc7cc(-c8ccccc8O)nnc7N)cn6)CC5)C4)CC3)CC2)no1 ; CC(C)[C@@H](C(=O)N1C[C@H](O)C[C@H]1C(=O)N[C@@H](C)c1ccc(-c2ccnn2C)cc1)c1cc(N2CCC(OC3CCN(CC4CC(N5CCC([C@H](C)n6cc(C#Cc7cc(-c8ccccc8O)nnc7N)cn6)CC5)C4)CC3)CC2)no1] 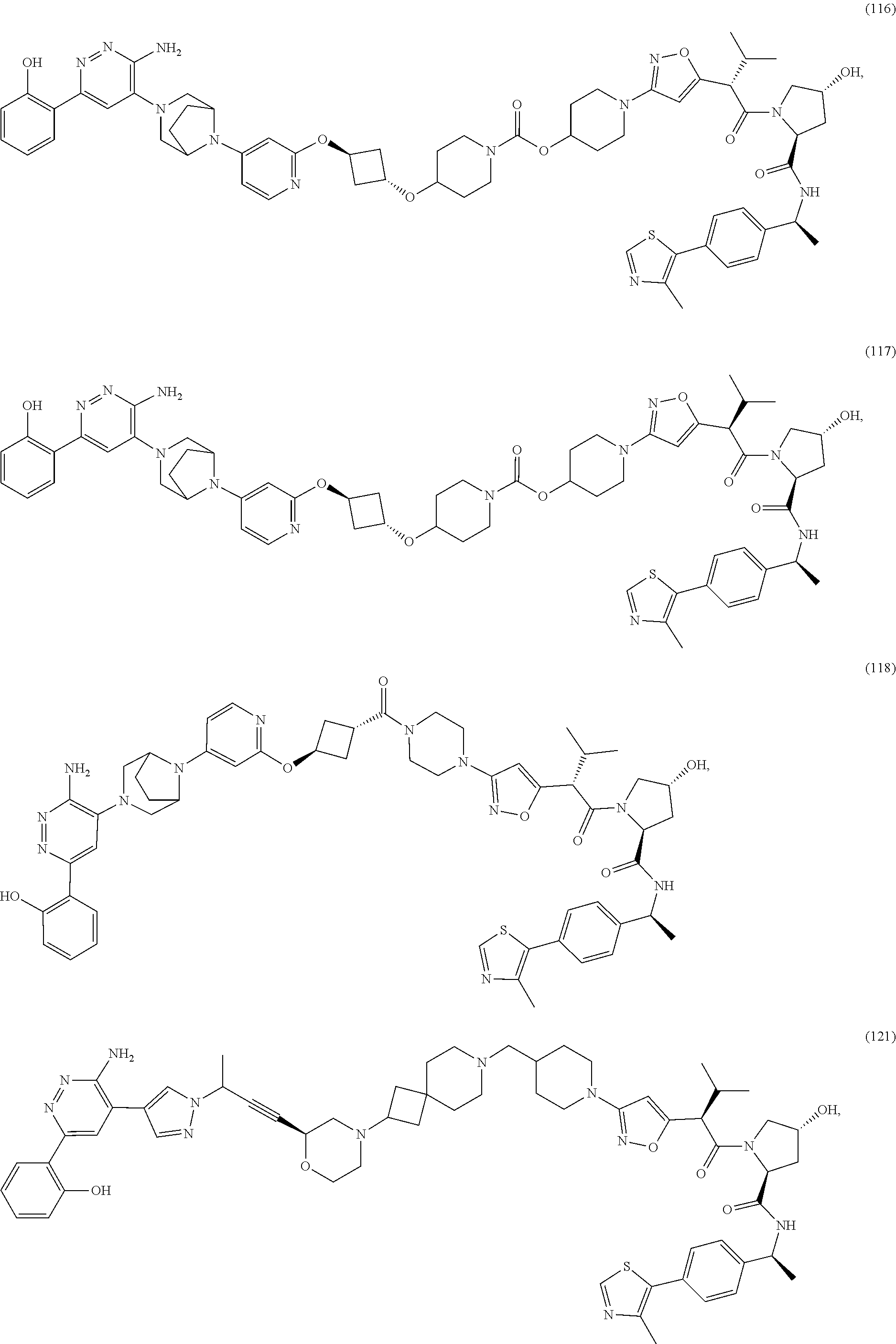 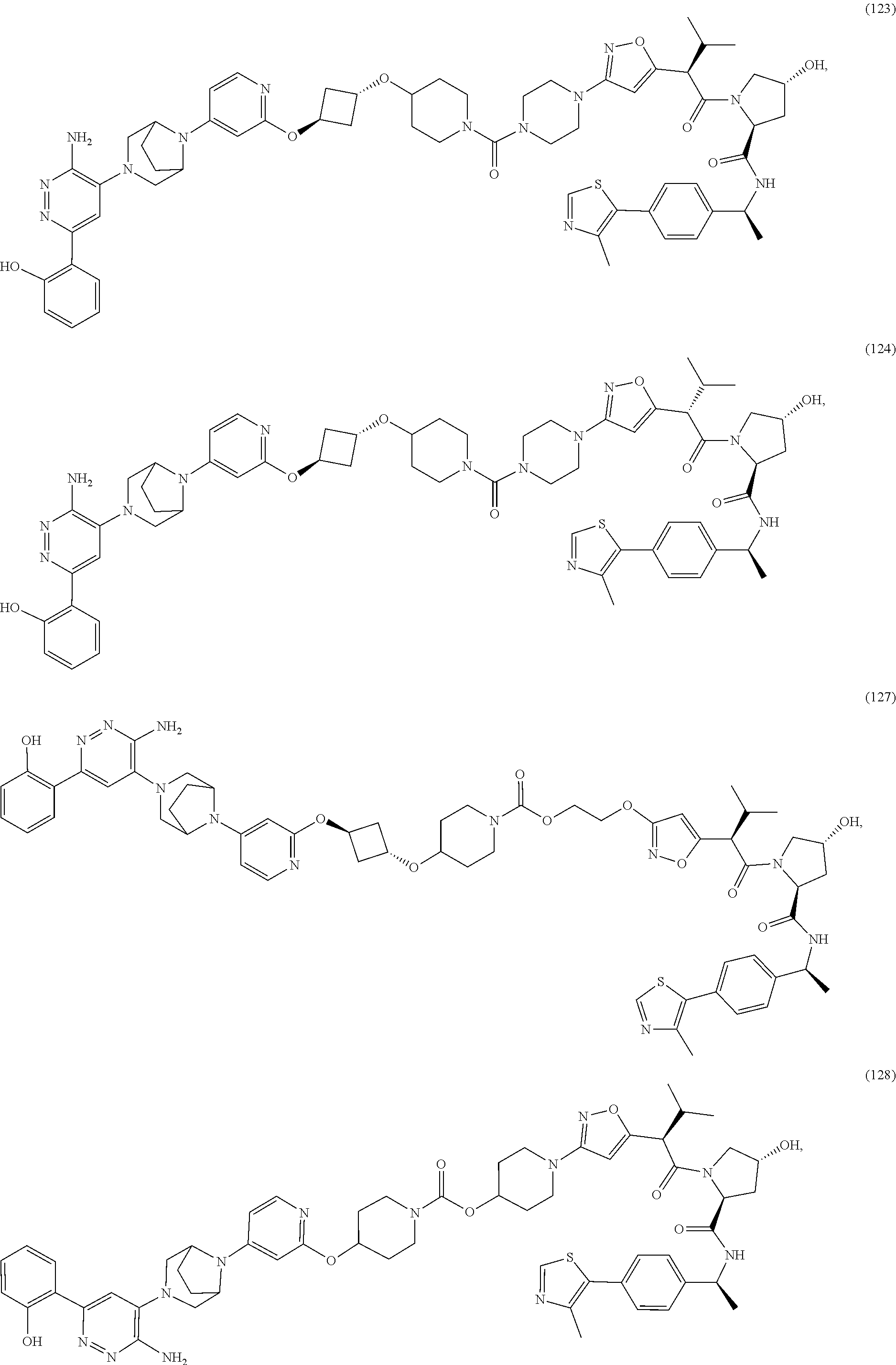 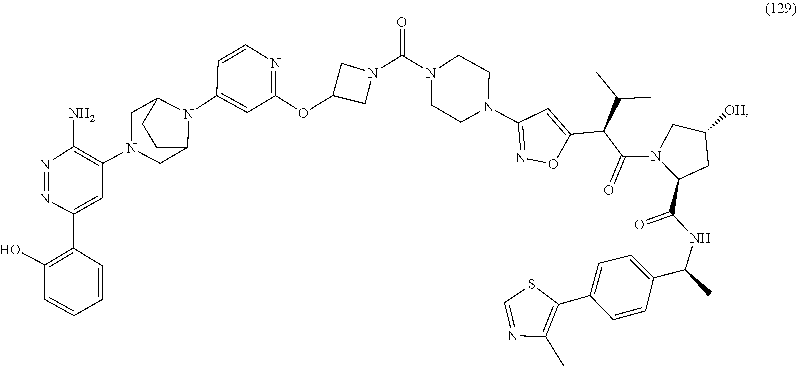 [SMILES: Cc1ncsc1-c1ccc([C@H](C)NC(=O)[C@@H]2C[C@@H](O)CN2C(=O)[C@@H](c2cc(N3CCN(C(=O)N4CC(Oc5cc(N6C7CCC6CN(c6cc(-c8ccccc8O)nnc6N)C7)ccn5)C4)CC3)no2)C(C)C)cc1] 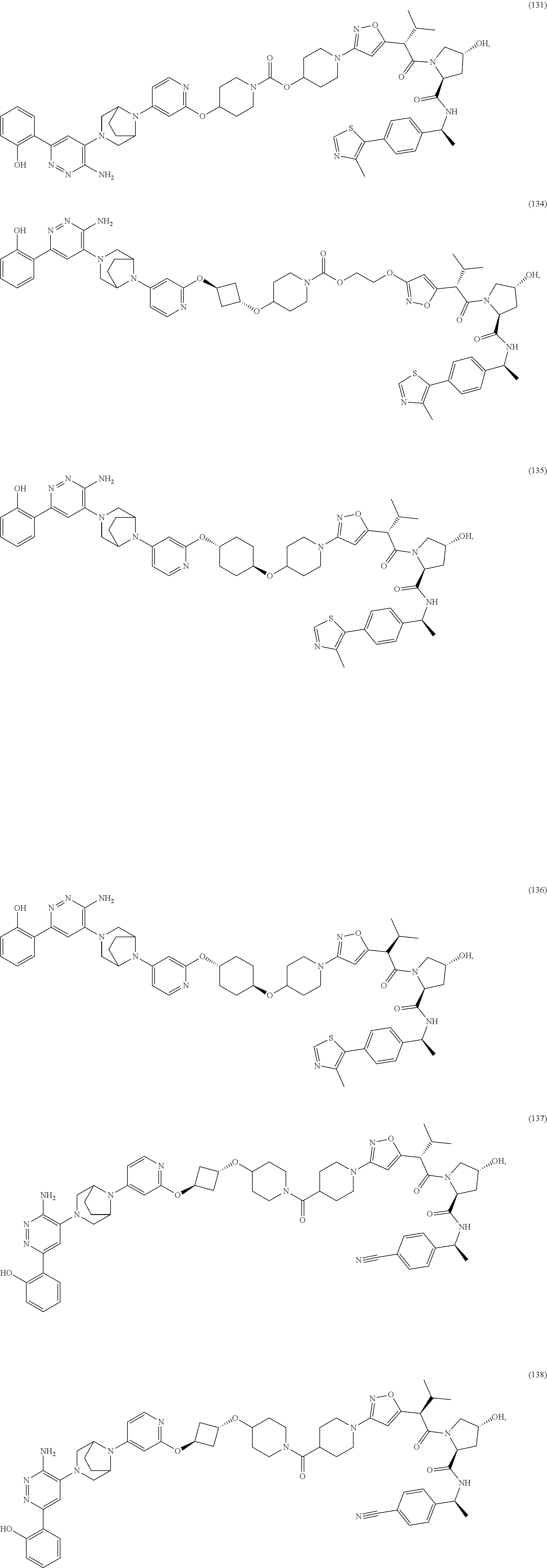 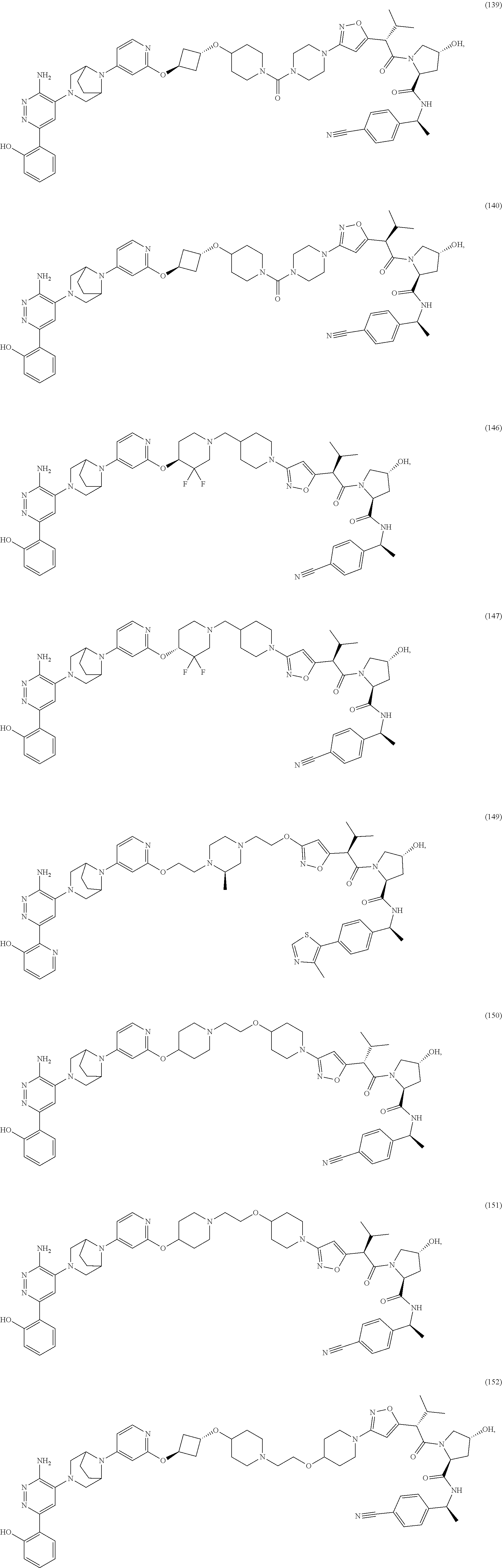 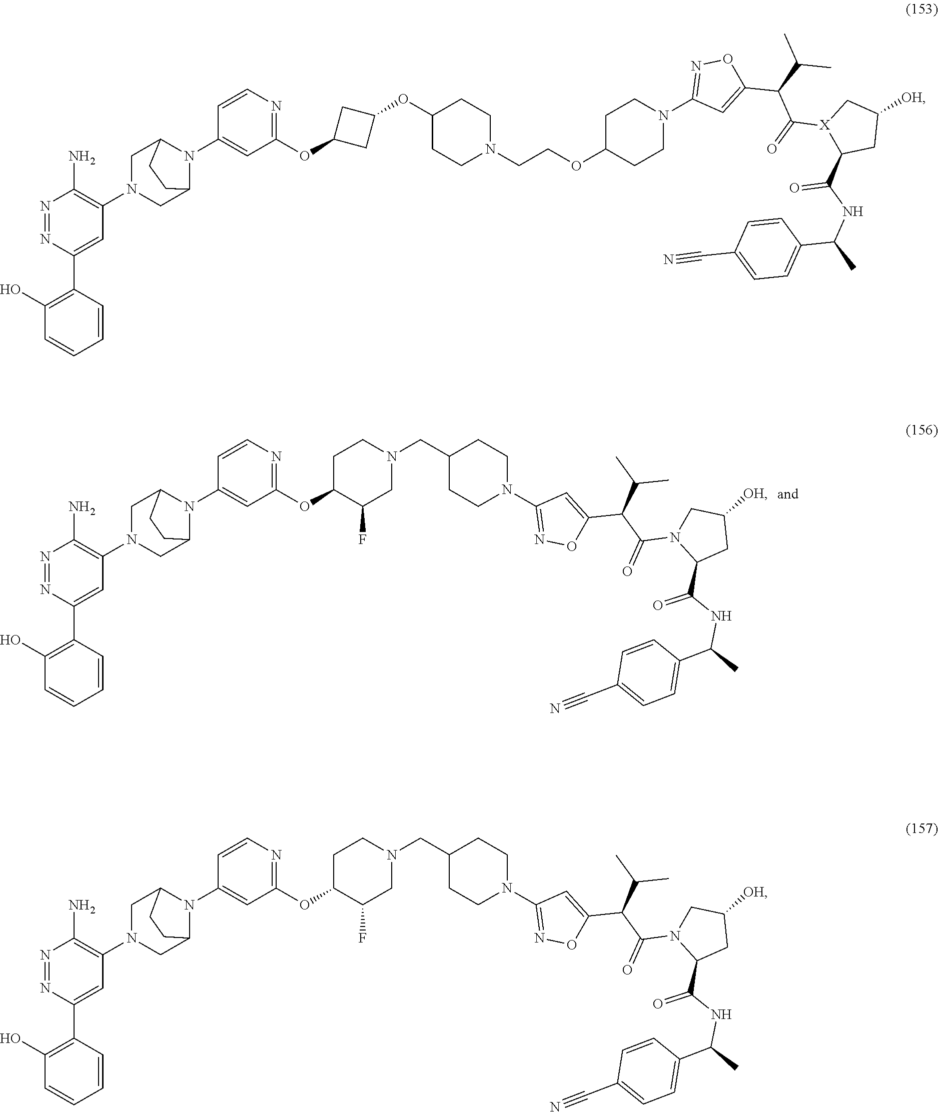
or a pharmaceutically acceptable salt thereof.

29. The compound of any of claims 26-28, wherein at least one of: (i) the compound has a Dₘₐₓ greater than 50%, greater than 75%, or greater than or equal to 80%, (ii) the compound has a DC₅₀ less than 10 nM or less than 2.5 nM, or (iii) both (i) and (ii).

30. A pharmaceutical composition comprising an effective amount of a bifunctional compound of any of claims 1-29 and a pharmaceutically acceptable carrier.

31. The pharmaceutical composition of claim 30, further comprising an anti-cancer agent.

32. A composition comprising a pharmaceutically acceptable carrier and an effective amount of at least one compound of any of claims 1-29 for treating a disease or disorder in a subject, the method comprising administering the composition to a subject in need thereof, wherein the compound is effective in treating or ameliorating at least one symptom of the disease or disorder, wherein the disease or disorder is associated with SMARCA1, BRAHMA or BRM accumulation and aggregation.

33. The composition of claim 32, wherein the disease or disorder is cancer.

34. The composition of claim 33, wherein the cancer is a SWI/SNF associated cancer or a cancer with a SMARCA4 mutation.

35. The composition of claim 34, wherein the SWI/SNF associated cancer or the cancer with a SMARCA4 mutation is lung cancer or non-small cell lung cancer.

36. The composition of claim 33, wherein the cancer is a SMARCA4-deficient cancer or a cancer with decreased expression of SMARCA4 relative to normal SMARCA4 expression.

37. The composition of claim 36, wherein the SMARCA4-deficient cancer or the cancer with decreased expression of SMARCA4 relative to normal SMARCA4 expression is lung cancer or non-small cell lung cancer.
